(19) United States
(12) Patent Application Publication
(10) Pub. No.: US 2021/0353543 A1
(43) Pub. Date: Nov. 18, 2021

(54) TARGETED LIPID PARTICLES AND COMPOSITIONS AND USES THEREOF

(71) Applicants: Sana Biotechnology, Inc., Seattle, WA (US); Flagship Pioneering Innovations V, Inc., Cambridge, MA (US)

(72) Inventors: Kyle Marvin TRUDEAU, Seattle, WA (US); Christopher BANDORO, Seattle, WA (US); Lauren Pepper MACKENZIE, Seattle, WA (US); Jagesh Vijaykumar SHAH, Seattle, WA (US); Geoffrey A. VON MALTZAHN, Somerville, MA (US); Jacob Rosenblum RUBENS, Cambridge, MA (US); Michael Travis MEE, Montreal (CA)

(73) Assignees: Sana Biotechnology, Inc., Seattle, WA (US); Flagship Pioneering Innovations V, Inc., Cambridge, MA (US)

(21) Appl. No.: 17/218,025

(22) Filed: Mar. 30, 2021

Related U.S. Application Data
(60) Provisional application No. 63/154,341, filed on Feb. 26, 2021.
(60) Provisional application No. 63/003,168, filed on Mar. 31, 2020.

(51) Int. Cl. A61K 9/127; C07K 14/005; C07K 16/28; C12N 15/86; C07K 14/725

(52) U.S. Cl. CPC A61K 9/1271; C07K 14/005; C07K 16/28; C12N 15/86; C07K 16/2803; C07K 14/7051; A61K 2039/505; C07K 16/2812; C07K 16/2815; C12N 2760/18222; C12N 2740/15043; C07K 2317/569; A61K 9/1277

(57) ABSTRACT

Provided herein are lipid particles containing a lipid bilayer enclosing a lumen or cavity, a henipavirus F protein molecule or biologically active portion thereof, and a targeted envelope protein containing a henipavirus envelope attachment glycoprotein G (G protein) or biologically active portion thereof and a binding domain, such as a single domain antibody (sdAb) variable domain. Also provided herein are targeted envelope proteins containing a G protein fused or linked to a binding domain, such as a sdAb variable domain, and polynucleotides encoding such proteins. Also provided are producer cells and compositions containing such targeted lipid particles and methods of making and using the targeted lipid particles.

### CROSS-REFERENCE TO RELATED APPLICATIONS

This application claims priority to U.S. provisional application 63/003,168 entitled “Targeted Lipid Particles and compositions and Uses Thereof”, filed Mar. 31, 2020, and to U.S. provisional application 63/154,341, entitled “Targeted Lipid Particles and compositions and Uses Thereof”, filed Feb. 26, 2021, the contents of each of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 186152003600SubSeqList.TXT, created Jun. 19, 2021, which is 2,076,399 bytes in size. The information in the electronic format of the Sequence Listing is incorporated by reference in its entirety

### FIELD

The present disclosure relates to lipid particles containing a lipid bilayer enclosing a lumen or cavity, a henipavirus F protein molecule or biologically active portion thereof, and a targeted envelope protein containing a henipavirus envelope attachment glycoprotein G (G protein) or biologically active portion thereof and a binding domain, such as a single domain antibody (sdAb) variable domain. The present disclosure also provides a targeted envelope protein containing a G protein fused or linked to a binding domain, such as a sdAb variable domain, and polynucleotides encoding such proteins. Also disclosed are producer cells and compositions containing such targeted lipid particles and methods of making and using the targeted lipid particles.

### BACKGROUND

Lipid particles, including virus-like particles and viral vectors, are commonly used for delivery of exogenous agents to cells. However, delivery of the lipid particles to certain target cells can be challenging. For lentivral vectors, the host range can be altered by pseudotyping with a heterologous envelope protein. Certain retargeted envelope proteins may not be sufficiently stable or expressed on the surface of the lipid particle. Improved lipid particles, including virus-like particles and viral vectors, for targeting desired cells are needed. The provided disclosure addresses this need.

### SUMMARY

Provided herein is a targeted lipid particle which includes (a) a lipid bilayer enclosing a lumen, (b) a henipavirus F protein molecule or biologically active portion thereof; and (c) a targeted envelope protein comprising (i) a henipavirus envelope attachment glycoprotein G (G protein) or a biologically active portion thereof and (ii) single domain antibody (sdAb) variable domain, wherein the sdAb variable domain is attached to the C-terminus of the G protein or the biologically active portion thereof, wherein the F protein molecule or the biologically active portion thereof and the targeted envelope protein are embedded in the lipid bilayer. In some embodiments, the the single domain antibody is attached to the G protein via a linker. In some embodiments, the linker is a peptide linker.

Provided herein is a targeted lipid particle which includes (a) a lipid bilayer enclosing a lumen, (b) a henipavirus F protein molecule or biologically active portion thereof; and (c) a targeted envelope protein comprising a henipavirus envelope attachment glycoprotein G (G protein) or biologically active portion thereof attached to a single domain antibody (sdAb) variable domain via a peptide linker, wherein the single domain antibody binds to a cell surface molecule of a target cell, wherein the F protein molecule or biologically active portion thereof and the targeted envelope protein are embedded in the lipid bilayer. In some embodiments, N-terminus of the F protein molecule or biologically active portion thereof is exposed on the outside of lipid bilayer. In some embodiments, the C-terminus of the G protein is exposed on the outside of the lipid bilayer.

In some embodiments, the single domain antibody binds a cell surface molecule present on a target cell. In some embodiments, the cell surface molecule is a protein, glycan, lipid or low molecular weight molecule. In some of any embodiments, the single domain antibody binds an antigen or portion thereof present on a target cell. In some embodiments, the antigen is the cell surface molecule or a portion of the cell surface molecule that contains an epitope recognized by the single domain antibody. In some of any embodiments, the target cell is selected from the group consisting of tumor-infiltrating lymphocytes, T cells, neoplastic or tumor cells, virus-infected cells, stem cells, central nervous system (CNS) cells, hematopoeietic stem cells (HSCs), liver cells or fully differentiated cells. In some embodiments, the target cell is selected from the group consisting of a CD3+ T cell, a CD4+ Tcell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell. In some of any embodiments, the target cell is a hepatocyte. In some of any embodiments, the cell surface molecule or antigen is selected from the group consisting of ASGR1, ASGR2 and TM4SF5.

In some of any embodiments, the target cell is a T cell. In some of any embodiments, the cell surface molecule or antigen is CD8 or CD4.

In some of any embodiments, the cell surface molecule or antigen is LDL-R.

Provided herein are targeted lipid particles comprising (a) a lipid bilayer enclosing a lumen, (b) a henipavirus F protein molecule or biologically active portion thereof; and (c) a targeted envelope protein comprising (i) a henipavirus envelope attachment glycoprotein G (G protein) or a biologically active portion thereof and (ii) a binding domain, wherein the binding domain is attached to the C-terminus of the G protein or the biologically active portion thereof, and wherein the binding domain binds a cell surface molecule selected from the group consisting of ASGR1, ASGR2, and TM4SF5, optionally human ASGR1, human ASGR2 and human ASGR2,

wherein the F protein molecule or the biologically active portion thereof and the targeted envelope protein are embedded in the lipid bilayer.

Provided herein are targeted lipid particles comprising (a) a lipid bilayer enclosing a lumen, (b) a henipavirus F protein molecule or biologically active portion thereof; and (c) a targeted envelope protein comprising (i) a henipavirus envelope attachment glycoprotein G (G protein) or a biologically active portion thereof and (ii) a binding domain, wherein the binding domain is attached to the C-terminus of the G protein or the biologically active portion thereof, and wherein the binding domain binds a cell surface molecule selected from the group consisting of CD8 and CD4, optionally human CD8 or human CD4, wherein the F protein molecule or the biologically active portion thereof and the targeted envelope protein are embedded in the lipid bilayer.

Provided herein are targeted lipid particles comprising (a) a lipid bilayer enclosing a lumen, (b) a henipavirus F protein molecule or biologically active portion thereof; and (c) a targeted envelope protein comprising (i) a henipavirus envelope attachment glycoprotein G (G protein) or a biologically active portion thereof and (ii) a binding domain, wherein the binding domain is attached to the C-terminus of the G protein or the biologically active portion thereof, and wherein the binding domain binds a cell surface molecule that is low density lipoprotein receptor (LDL-R), optionally human LDL-R, wherein the F protein molecule or the biologically active portion thereof and the targeted envelope protein are embedded in the lipid bilayer.

In some of any embodiments, the lipid particle is a lentiviral vector. In some of any embodiments, the binding domain is attached to the G protein via a linker. In some of any embodiments, the linker is a peptide linker.

Provided herein is a lentiviral vector, comprising a binding domain that targets a cell surface molecule selected from the group consisting of ASGR1, ASGR2 and TM4SF5, optionally human ASGR1, human ASGR2 and human TM4SF5, wherein the lentiviral vector is pseudotyped with a retargeted viral fusion protein, said retargeted viral fusion protein comprising: (a) a henipavirus F protein molecule or biologically active portion thereof; and (b) a targeted envelope protein comprising the binding domain attached to a henipavirus envelope attachment glycoprotein G (G protein) or a biologically active portion thereof.

Provided herein is a lentiviral vector, comprising a binding domain that targets a cell surface molecule selected from the group consisting of CD8 and CD4, optionally human CD8 and human CD4, wherein the lentiviral vector is pseudotyped with a retargeted viral fusion protein, said retargeted viral fusion protein comprising: (a) a henipavirus F protein molecule or biologically active portion thereof; and (b) a targeted envelope protein comprising the binding domain attached to a henipavirus envelope attachment glycoprotein G (G protein) or a biologically active portion thereof.

Provided herein is a lentiviral vector, comprising a binding domain that targets low density lipoprotein receptor (LDL-R), optionally wherein the LDL-R is human LDL-R, wherein the lentiviral vector is pseudotyped with a retargeted viral fusion protein comprising (a) a henipavirus F protein molecule or biologically active portion thereof; and (b) a targeted envelope protein comprising the binding domain attached to a henipavirus envelope attachment glycoprotein G (G protein) or a biologically active portion thereof.

In some of any embodiments, the binding domain is attached to the C-terminus of the G protein or the biologically active portion thereof.

Provided herein is a lentiviral vector, comprising (a) a henipavirus F protein molecule or biologically active portion thereof; and (b) a targeted envelope protein comprising (i) a henipavirus envelope attachment glycoprotein G (G protein) or a biologically active portion thereof and (ii) a binding domain, wherein the binding domain is attached to the C-terminus of the G protein or the biologically active portion thereof, and wherein the binding domain binds CD4; and (c) a cargo comprising nucleic acid encoding a chimeric antigen receptor (CAR), wherein the CAR comprises (i) an extracellular antigen binding domain that binds an extracellular antigen (e.g., CD19 or BCMA) and (ii) an intracellular signaling region a CD3zeta signaling domain and, optionally a 4-1BB or CD28 co-stimulatory signaling domain. In some embodiments, the extracellular antigen binding domain of the CAR is an scFv.

In some of any embodiments, the lentiviral vector is capable of delivering the nucleic acid encoding the CAR to T cells. In some embodiments the T cells are in vivo in a subject.

Provided herein is a lentiviral vector, comprising:(a) a henipavirus F protein molecule or biologically active portion thereof; and (b) a targeted envelope protein comprising (i) a henipavirus envelope attachment glycoprotein G (G protein) or a biologically active portion thereof and (ii) a binding domain, wherein the binding domain is attached to the C-terminus of the G protein or the biologically active portion thereof, and wherein the binding domain binds ASGR1; wherein the lentiviral vector is capable of targeting to hepatocytes. In some of any embodiments, the lentiviral vector further comprises an exogenous agent for delivery to hepatocytes.

In some of any embodiments, the lentiviral vector is capable of delivering the exogenous agent to hepatocytes, optionally wherein the hepatocytes are in vivo in a subject.

In some of any embodiments, the binding domain is attached to the G protein via a linker. In some of any embodiments, the linker is a peptide linker. In some of any embodiments, the binding domain is a single domain antibody. In some of any embodiments, the binding domain is a single chain variable fragment (scFv).

In some of any embodiments, the peptide linker comprises up to 65 amino acids in length. In some of any embodiments, the peptide linker comprises up to 50 amino acids in length. In some of any embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some of any embodiments, peptide linker comprises a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 amino acids in length. In some of any embodiments, wherein the peptide linker is a flexible linker that comprises GS, GGS, GGGGS (SEQ ID NO:43), GGGGGS (SEQ ID NO:41) or combinations thereof. In some of any embodiments, the peptide linker comprises (GGS)n, wherein n is 1 to 10. In some of any embodiments, the peptide linker comprises (GGGGS)n (SEQ ID NO: 42), wherein n is 1 to 10. In some of any embodiments, the peptide linker comprises (GGGGGS)n (SEQ ID NO:27), wherein n is 1 to 6.

In some of any embodiments, the G protein or the biologically active portion thereof is a wild-type Nipah virus G (NiV-G) protein or a Hendra virus G protein. In some of any embodiments, the G protein or the biologically active portion thereof is a wild-type NiV-G protein or a functionally active variant or biologically active portion thereof. In some of any embodiments, the mutant NiV-G protein or functionally active variant or biologically active portion thereof comprises an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:9, SEQ ID NO:28 or SEQ ID NO:44.

In some of any embodiments, the NiV-G protein is a biologically active portion that is truncated and lacks up to 40 contiguous amino acid residues at or near the N-terminus of the wild-type NiV-G protein (SEQ ID NO:9, SEQ ID NO:28 or SEQ ID NO:44).

In some of any embodiments, the NiV-G protein is a biologically active portion that is truncated at the N-terminus of wild-type NiV-G and has the sequence set forth in any of SEQ ID NOS: 10-15, 35-40 or 45-50 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs: 10-15, 35-40 or 45-50.

In some of any embodiments, the NiV-G protein is a biologically active portion that has a 5 amino acid truncation at or near the N-terminus of the wild-type NiV-G protein (SEQ ID NO:9, SEQ ID NO:28 or SEQ ID NO:44). In some of any embodiments, the NiV-G protein or the biologically active portion has the amino acid sequence set forth in SEQ ID NO: 10 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:10. In some of any embodiments, the NiV-G protein or the biologically active portion has the amino acid sequence set forth in SEQ ID NO: 35 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:35. In some of any embodiments, the NiV-G protein or the biologically active portion has the amino acid sequence set forth in SEQ ID NO: 45 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:45.

In some of any embodiments, the NiV-G protein is a biologically active portion that has a 10 amino acid truncation at or near the N-terminus of the wild-type NiV-G protein (SEQ ID NO:9, SEQ ID NO:28 or SEQ ID NO:44). In some of any embodiments, the NiV-G protein or the biologically active portion has the amino acid sequence set forth in SEQ ID NO: 36 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:36. In some of any embodiments, the NiV-G protein or the biologically active portion has the amino acid sequence set forth in SEQ ID NO: 11 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:11. In some of any embodiments, the NiV-G protein or the biologically active portion has the amino acid sequence set forth in SEQ ID NO: 46 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46.

In some of any embodiments, the NiV-G protein or the biologically active portion has a 15 amino acid truncation at or near the N-terminus of the wild-type NiV-G protein (SEQ ID NO:9, SEQ ID NO:28 or SEQ ID NO:44). In some of any embodiments, the NiV-G protein or the biologically active portion has the amino acid sequence set forth in SEQ ID NO: 12 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12. In some of any embodiments, the NiV-G protein or the biologically active portion has the amino acid sequence set forth in SEQ ID NO: 37 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:37. In some of any embodiments, the NiV-G protein or the biologically active portion has the amino acid sequence set forth in SEQ ID NO: 47 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:47.

In some of any embodiments, the NiV-G protein is a biologically active portion that has a 20 amino acid truncation at or near the N-terminus of the wild-type NiV-G protein (SEQ ID NO:9, SEQ ID NO:28 or SEQ ID NO:44). In some of any embodiments, the NiV-G protein or the biologically active portion has the amino acid sequence set forth in SEQ ID NO: 13 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:13. In some of any embodiments, the NiV-G protein or the biologically active portion has the amino acid sequence set forth in SEQ ID NO: 38 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:38. In some of any embodiments, the NiV-G protein or the biologically active portion has the amino acid sequence set forth in SEQ ID NO: 48 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:48.

In some of any embodiments, the NiV-G protein is a biologically active portion has a 25 amino acid truncation at or near the N-terminus of the wild-type NiV-G protein (SEQ ID NO:9, SEQ ID NO:28 or SEQ ID NO:44). In some of any embodiments, the NiV-G protein has the amino acid sequence set forth in SEQ ID NO: 14 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14. In some of any embodiments, the NiV-G protein or the biologically active portion has the amino acid sequence set forth in SEQ ID NO: 39 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:39. In some of any embodiments, the NiV-G protein or the biologically active portion has the amino acid sequence set forth in SEQ ID NO: 49 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:49.

In some of any embodiments, the NiV-G protein is a biologically active portion has a 30 amino acid truncation at or near the N-terminus of the wild-type NiV-G protein (SEQ ID NO:9, SEQ ID NO:28 or SEQ ID NO:44). In some of any embodiments, the NiV-G protein or the biologically active portion has the amino acid sequence set forth in SEQ ID NO: 15 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15. In some of any embodiments, the NiV-G protein or the biologically active portion has the amino acid sequence set forth in SEQ ID NO: 40 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:40.

In some of any embodiments, the NiV-G protein is a biologically active portion that has a 34 amino acid truncation at or near the N-terminus of the wild-type NiV-G protein (SEQ ID NO:9, SEQ ID NO:28 or SEQ ID NO:44). In some of any embodiments, the NiV-G protein or the biologically active portion has the amino acid sequence set forth in SEQ ID NO: 22 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:22. In some of any embodiments, the NiV-G protein or the biologically active portion has the amino acid sequence set forth in SEQ ID NO: 53 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:53.

In some of any embodiments, the G-protein, the biologically active portion thereof is a functionally active variant that is a mutant NiV-G protein that exhibits reduced binding to Ephrin B2 or Ephrin B3.

In some of any embodiments, the mutant NiV-G protein includes one or more amino acid substitutions corresponding to amino acid substitutions selected from the group consisting of E501A, W504A, Q530A and E533A with reference to numbering set forth in SEQ ID NO:28. In some of any embodiments, the mutant NiV-G protein includes the amino acid substitutions E501A, W504A, Q530A and E533A with reference to numbering set forth in SEQ ID NO:28.

In some of any embodiments, the mutant NiV-G protein or the biologically active portion has the amino acid sequence set forth in SEQ ID NO: 16 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:16. In some of any embodiments, the mutant NiV-G protein or the biologically active portion has the amino acid sequence set forth in SEQ ID NO: 51 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:51.

In some of any embodiments, the F protein or the biologically active portion thereof is a wild-type Nipah virus F (NiV-F) protein or a Hendra virus F protein or is a functionally active variant or biologically active portion thereof. In some of any embodiments, the F protein or the biologically active portion thereof is a wild-type NiV-F protein or a functionally active variant or a biologically active portion thereof. In some of any embodiments, the NiV-F-protein or the functionally active variant or biologically active portion thereof comprises the amino acid sequence set forth in SEQ ID NO: 2, or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 2.

In some of any embodiments, the NiV-F protein is a biologically active portion thereof that has a 20 amino acid truncation at or near the C-terminus of the wild-type NiV-F protein (SEQ ID NO:2).

In some of any embodiments, the NiV-F protein or the biologically active portion has the sequence set forth in SEQ ID NO:5 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 5.

In some of any embodiments, the NiV-F protein is a biologically active portion thereof that includes i) a 20 amino acid truncation at or near the C-terminus of the wild-type NiV-F protein (SEQ ID NO:2); and ii) a point mutation on an N-linked glycosylation site.

In some of any embodiments, the NiV-F protein or the biologically active portion has the sequence set forth in SEQ ID NO:7 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 7.

In some of any embodiments, the NiV-F protein is a biologically active portion thereof that has a 22 amino acid truncation at or near the C-terminus of the wild-type NiV-F protein (SEQ ID NO:2).

In some of any embodiments, NiV-F protein or the biologically active portion has the sequence set forth in SEQ ID NO:8 or an amino acid sequence that is encoded by a sequence of nucleotides encoding a sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 8.

In some of any embodiments, the NiV-F protein or the biologically active portion has the sequence set forth in SEQ ID NO:23 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 23. In some of any embodiments, the F-protein or the biologically active portion thereof comprises an F1 subunit or a fusogenic portion thereof.

In some of any embodiments, the F protein comprises the sequence set forth in SEQ ID NO:23 and the G protein comprises the sequence set forth in SEQ ID NO:16.

In some of any embodiments, the F protein consists or consists essentially of the sequence set forth in SEQ ID NO:23 and/or the G protein consists or consists essentially of the sequence set forth in SEQ ID NO:16.

In some of any embodiments, the F1 subunit is a proteolytically cleaved portion of the F0 precursor. In some of any embodiments, the F1 subunit comprises the sequence set forth in SEQ ID NO: 4, or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:4.

In some of any embodiments, the lipid bilayer is derived from a membrane of a host cell used for producing a retrovirus or retrovirus-like particle. In some of any embodiments, the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRCS cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In some of any embodiments, the host cell comprises 293T cells. In some of any embodiments, the lipid bilayer is or comprises a viral envelope. In some of any embodiments, the retrovirus-like particle is replication defective.

In some of any embodiments, the targeted lipid particle comprises one or more viral components other than the F protein molecule and the G protein. In some of any embodiments, the one or more viral components are from a retrovirus. In some of any embodiments, the retrovirus is a lentivirus. In some of any embodiments, the one or more viral components comprise a viral packaging protein selected from one or more of Gag, Pol, Rev and Tat. In some of any embodiments, the one or more viral components comprises one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

In some of any embodiments, the targeted lipid particle is a lentiviral vector.

In some of any embodiments, the targeted lipid particle or the lentiviral vector is replication defective.

In some of any embodiments, the targeted lipid particle or the lentiviral vector further comprises an exogenous agent. In some of any embodiments, the targeted lipid particle further comprises an exogenous agent. In some embodiments, the lentiviral vector further comprises an exogenous agent.

In some of any embodiments, the exogenous agent is present in the lumen. In some of any embodiments, the exogenous agent is a protein or a nucleic acid. In some embodiments, the nucleic acid is a DNA or RNA.

In some of any embodiments, the exogenous agent is a nucleic acid encoding a cargo for delivery to the target cell. In some of any embodiments, the exogenous agent encodes a therapeutic agent or a diagnostic agent.

In some of any embodiments, the exogenous agent encodes a membrane protein. In some embodiments, the membrane protein is an antigen receptor for targeting cells expressed by or associated with a disease or condition. In some embodiments, the membrane protein is a chimeric antigen receptor (CAR). In some embodiments, the CAR comprises (i) an extracellular antigen binding domain that binds an extracellular antigen (e.g., CD19 or BCMA), optionally wherein the extracellular antigen binding domain is an scFv, (ii) a transmembrane domain and (iii) an intracellular signaling region comprising a CD3zeta signaling domain and, optionally a co-stimulatory signaling domain, e.g., a 4-1BB or CD28 co-stimulatory signaling domain. In some embodiments, the target cell is a T cell. In some embodiments, the cell surface molecule on the target cell is CD4 or CD8. In some embodiments, the binding domain is an scFv that binds CD4 (e.g. human CD4). In some embodiments, the binding domain is a single domain antibody that binds CD4 (e.g. human CD4). In some embodiments, the binding domain is an scFv that binds CD8 (e.g. human CD8). In some embodiments, the binding domain is a single domain antibody that binds CD8 (e.g. human CD8).

In some of any embodiments, the exogenous agent is a nucleic acid comprising a payload gene for correcting a genetic deficiency, optionally a genetic deficiency in the target cell. In some embodiments, the genetic deficiency is associated with a liver cell or a hepatocyte. In some embodiments, the target cell is a hepatocyte. In some embodiments, the cell surface molecule is a molecule selected from the group consisting of ASGR1, ASGR2 and TM4SF5. In some embodiments, the binding domain is an scFv that binds ASGR1 (e.g. human ASGR1). In some embodiments, the binding domain is a single domain antibody that binds ASGR1 (e.g. human ASGR1). In some embodiments, the binding domain is an scFv that binds ASGR2 (e.g. human ASGR2). In some embodiments, the binding domain is a single domain antibody that binds ASGR2 (e.g. human ASGR2). In some embodiment, the binding domain is a scFv that binds TM4SF5 (e.g. human TM4SF5). In some embodiments, the binding domain is a single domain antibody that binds TM4SF5 (e.g. human TM4SF5).

In some of any embodiments, the single domain antibody binds a cell surface molecule present on a target cell. In some of any embodiments, the cell surface molecule is a protein, glycan, lipid or low molecular weight molecule. In some of any embodiments, the target cell is selected from the group consisting of tumor-infiltrating lymphocytes, T cells, neoplastic or tumor cells, virus-infected cells, stem cells, central nervous system (CNS) cells, hematopoeietic stem cells (HSCs), liver cells or fully differentiated cells. In some of any embodiments, the target cell is selected from the group consisting of a CD3+ T cell, a CD4+ Tcell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some of any embodiments, the single domain antibody binds an antigen or portion thereof present on a target cell. In some of any embodiments, the cell surface molecule or antigen is selected from the group consisting of ASGR1, ASGR2 and TM4SF5. In some embodiments, the antigen or portion thereof is human ASGR1. In some embodiments, the antigen or portion thereof is human ASGR2. In some embodiments, the antigen or portion thereof is human TM4SF5.

Provided herein is a polynucleotide comprising a nucleic acid sequence encoding (i) a henipavirus envelope attachment glycoprotein G (G protein) or a biologically active portion thereof and (ii) a binding domain that binds a cell surface molecule selected from the group consisting of ASGR1, ASGR2, and TM4SF5. In some embodiments, the cell surface molecule is human ASGR1. In some embodiments, the cell surface molecule is human ASGR2. In some embodiments, the cell surface molecule is human TM4SF5. In some of any embodiments, the cell surface molecule or antigen is CD8 or CD4.

Provided herein is a nucleic acid sequence encoding (i) a henipavirus envelope attachment glycoprotein G (G protein) or a biologically active portion thereof and (ii) a binding domain that binds a cell surface molecule selected from the group consisting of CD4 and CD8. In some embodiments, the cell surface molecule is human CD4. In some embodiments, the cell surface molecule is human CD8. In some embodiments, the cell surface molecule or antigen is low density lipoprotein receptor (LDL-R). In some embodiments, the cell surface molecule or antigen is human LDL-R.

Provided herein is a polynucleotide comprising a nucleic acid sequence encoding (i) a henipavirus envelope attachment glycoprotein G (G protein) or a biologically active portion thereof and (ii) a binding domain that binds low density lipoprotein receptor (LDL-R). In some embodiments, the binding domain binds human LDL-R. In some of any embodiments, the binding domain is a single domain antibody (sdAb). In some of any embodiments, the binding domain is a single chain variable fragment (scFv).

Provided herein is a polynucleotide comprising a nucleic acid sequence encoding (i) a henipavirus envelope attachment glycoprotein G (G protein) or a biologically active portion thereof and (ii) a single domain antibody (sdAb) variable domain, wherein the sdAb variable domain is attached to the C-terminus of the G protein or the biologically active portion thereof. In some of any embodiments, the polynucleotide further comprises (iii) a nucleic acid sequence encoding a henipavirus F protein molecule or a biologically active portion thereof.

In some embodiments, the nucleic acid sequence is a first nucleic acid sequence and the polynucleotide further comprise a second nucleic acid sequence encoding a henipavirus F protein molecule or a biologically active portion thereof. In some embodiments, the polynucleotide comprise an IRES or a sequence encoding a linking peptide between the first and second nucleic acid sequence. In some embodiments, the linking peptide is a self-cleaving peptide or a peptide that causes ribosome skipping, optionally a T2A peptide.

In some of any embodiments, the polynucleotide includes at least one promoter that is operatively linked to control expression of the nucleic acid. In some of any embodiments, the promoter is operatively linked to control expression of the first nucleic acid sequence and the second nucleic acid sequence. In some of any embodiments, the promoter is a constitutive promoter. In some of any embodiments, the promoter is an inducible promoter.

In some of any embodiments, the sdAb variable domain is attached to the G protein via an encoded peptide linker. In some embodiments, the binding domain is attached to the G protein via an encoded peptide linker. In some of any embodiments, the encoded peptide linker comprises up to 25 amino acids in length. In some of any embodiments, the encoded peptide linker comprises up to 65 amino acids in length In some of any embodiments, the encoded peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids.

In some of any embodiments, the encoded peptide linker comprises a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 amino acids in length. In some of any embodiments, the encoded peptide linker comprises GS, GGS, GGGGS (SEQ ID NO:43), GGGGGS (SEQ ID NO:41) and combinations thereof. In some of any embodiments, the encoded peptide linker comprises (GGS)n, wherein n is 1 to 10. In some of any embodiments, the encoded peptide linker comprises (GGGGS)n (SEQ ID NO:42), wherein n is 1 to 10. In some of any embodiments, the encoded peptide linker comprises (GGGGGS)n (SEQ ID NO:27), wherein n is 1 to 4. In some of any embodiments, the sequence encoding the G protein is a wild-type Nipah virus G (NiV-G) protein or a Hendra virus G protein or is a functionally active variant or a biologically active portion thereof. In some embodiments, the variant is a variant thereof that exhibits reduced binding for the native binding partner. In some of any embodiments, the nucleic acid sequence encoding the G protein is a wild-type Nipah virus G (NiV-G) protein or a Hendra virus G protein or is a variant thereof that exhibits reduced binding for the native binding partner. In some embodiments, the encoded G protein is a wild-type NiV-G protein or a functionally active variant or a biologically active portion thereof. In some of any embodiments, the nucleic acid sequence encoding the G protein is a wild-type NiV-G protein. In some of any embodiments, the nucleic acid sequence encoding the G-protein is a mutant NiV-G protein that exhibits reduced binding to Ephrin B2 or Ephrin B3.

In some of any embodiments, the NiV-G protein or functionally active variant or biologically active portion thereof comprises the amino acid sequence set forth in SEQ ID NO:9, SEQ ID NO: 28 or SEQ ID NO: 44 or comprises an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:9, SEQ ID NO:28 or SEQ ID NO:44. In some of any embodiments, the NiV-G protein is a biologically active portion that is truncated and lacks up to 40 contiguous amino acid residues at or near the N-terminus of the wild-type NiV-G protein (SEQ ID NO:9, SEQ ID NO:28 or SEQ ID NO:44). In some of any embodiments, the NiV-G protein is a biologically active portion that is truncated at the N-terminus of wild-type NiV-G and comprises the sequence set forth in any of SEQ ID NOS: 10-15, 35-40 or 45-50 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs: 10-15, 35-40 or 45-50.

In some of any embodiments, the NiV-G protein is a biologically active portion that comprises a 5 amino acid truncation at or near the N-terminus of the wild-type NiV-G protein (SEQ ID NO:9, SEQ ID NO:28 or SEQ ID NO:44). In some of any embodiments, the NiV-G protein or the biologically active portion comprises the amino acid sequence set forth in SEQ ID NO: 10 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:10. In some of any embodiments, NiV-G protein or the biologically active portion comprises the amino acid sequence set forth in SEQ ID NO: 35 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:35. In some of any embodiments, the NiV-G protein or the biologically active portion comprises the amino acid sequence set forth in SEQ ID NO: 45 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:45.

In some of any embodiments, NiV-G protein is a biologically active portion that comprises a 10 amino acid truncation at or near the N-terminus of the wild-type NiV-G protein (SEQ ID NO:9, SEQ ID NO:28 or SEQ ID NO:44). In some of any embodiments, the mutant NiV-G protein or the biologically active portion comprises the amino acid sequence set forth in SEQ ID NO: 11 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:11. In some of any embodiments, the NiV-G protein or the biologically active portion comprises the amino acid sequence set forth in SEQ ID NO: 36 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:36. In some of any embodiments, the NiV-G protein or the biologically active portion comprises the amino acid sequence set forth in SEQ ID NO: 46 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46.

In some of any embodiments, the is a biologically active portion that NiV-G protein comprises a 15 amino acid truncation at or near the N-terminus of the wild-type NiV-G protein (SEQ ID NO:9, SEQ ID NO:28 or SEQ ID NO:44). In some of any embodiments, the NiV-G protein or the biologically active portion comprises the amino acid sequence set forth in SEQ ID NO: 12 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12. In some of any embodiments, the NiV-G protein or the biologically active portion comprises the amino acid sequence set forth in SEQ ID NO: 37 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:37. In some of any embodiments, the NiV-G protein or the biologically active portion comprises the amino acid sequence set forth in SEQ ID NO: 47 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:47.

In some of any embodiments, the NiV-G protein is a biologically active portion that comprises a 20 amino acid truncation at or near the N-terminus of the wild-type NiV-G protein (SEQ ID NO:9, SEQ ID NO:28 or SEQ ID NO:44). In some of any embodiments, the NiV-G protein or the biologically active portion comprises the amino acid sequence set forth in SEQ ID NO: 13 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:13. In some of any embodiments, NiV-G protein or the biologically active portion comprises the amino acid sequence set forth in SEQ ID NO: 38 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:38. In some of any embodiments, the NiV-G protein or the biologically active portion comprises the amino acid sequence set forth in SEQ ID NO: 48 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:48.

In some of any embodiments, the NiV-G protein is a biologically active portion that comprises a 25 amino acid truncation at or near the N-terminus of the wild-type NiV-G protein (SEQ ID NO:9, SEQ ID NO:28 or SEQ ID NO:44). In some of any embodiments, the NiV-G protein or the biologically active portion comprises the amino acid sequence set forth in SEQ ID NO: 14 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14. In some of any embodiments, the NiV-G protein or the biologically active portion comprises the amino acid sequence set forth in SEQ ID NO: 39 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:39. In some of any embodiments, the NiV-G protein or the biologically active portion comprises the amino acid sequence set forth in SEQ ID NO: 49 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:49.

In some of any embodiments, the NiV-G protein is a biologically active portion that comprises a 30 amino acid truncation at or near the N-terminus of the wild-type NiV-G protein (SEQ ID NO:9, SEQ ID NO:28 or SEQ ID NO:44). In some of any embodiments, the NiV-G protein or the biologically active portion comprises the amino acid sequence set forth in SEQ ID NO: 15 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15. In some of any embodiments, the NiV-G protein or the biologically active portion comprises the amino acid sequence set forth in SEQ ID NO: 40 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:40. In some of any embodiments, the NiV-G protein or the biologically active portion comprises the amino acid sequence set forth in SEQ ID NO: 50 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 50.

In some of any embodiments, the NiV-G protein is a biologically active portion that has a 34 amino acid truncation at or near the N-terminus of the wild-type NiV-G protein (SEQ ID NO:9, SEQ ID NO:28 or SEQ ID NO:44). In some of any embodiments, the NiV-G protein or the biologically active portion has the amino acid sequence set forth in SEQ ID NO: 22 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:22. In some of any embodiments, the NiV-G protein or the biologically active portion has the amino acid sequence set forth in SEQ ID NO: 53 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:53.

In some of any embodiments, the G-protein is a mutant NiV-G protein that exhibits reduced binding to Ephrin B2 or Ephrin B3. In some of any embodiments, the mutant NiV-G protein comprises: one or more amino acid substitutions corresponding to amino acid substitutions selected from the group consisting of E501A, W504A, Q530A and E533A with reference to numbering set forth in SEQ ID NO:28. In some of any embodiments, the mutant NiV-G protein comprises amino acid substitutions E501A, W504A, Q530A and E533A with reference to numbering set forth in SEQ ID NO:28.

In some of any embodiments, the mutant NiV-G protein comprises: i) a truncation at or near the N-terminus; and ii) point mutations selected from the group consisting of E501A, W504A, Q530A and E533A. In some of any embodiments, the mutant NiV-G protein comprises the amino acid sequence set forth in SEQ ID NO: 16 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:16. In some of any embodiments, the mutant NiV-G protein comprises the amino acid sequence set forth in SEQ ID NO: 51 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:51.

In some of any embodiments, the F protein or the biologically active portion thereof is a wild-type Nipah virus F (NiV-F) protein or a Hendra virus F protein or is a functionally active variant or biologically active portion thereof. In some of any embodiments, the F protein or the biologically active portion thereof is a wild-type NiV-F protein or a functionally active variant or a biologically active portion thereof. In some of any embodiments, the NiV-F-protein or the functionally active variant or biologically active portion thereof comprises the amino acid sequence set forth in SEQ ID NO: 2, or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 2.

In some of any embodiments, the NiV-F protein is a is a biologically active portion thereof that has a 20 amino acid truncation at or near the C-terminus of the wild-type NiV-F protein (SEQ ID NO:2). In some of any embodiments, the NiV-F protein or the biologically active portion has the sequence set forth in SEQ ID NO:5 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 5. In some of any embodiments, the NiV-F protein is a biologically active portion thereof that comprises i) a 20 amino acid truncation at or near the C-terminus of the wild-type NiV-F protein (SEQ ID NO:2); and ii) a point mutation on an N-linked glycosylation site.

In some of any embodiments, the NiV-F protein or the biologically active portion has the sequence set forth in SEQ ID NO:7 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 7.

In some of any embodiments, the NiV-F protein is a biologically active portion thereof that has a 22 amino acid truncation at or near the C-terminus of the wild-type NiV-F protein (SEQ ID NO:2). In some of any embodiments, the NiV-F protein or the biologically active portion has the sequence set forth in SEQ ID NO:8 or an amino acid sequence that is encoded by a sequence of nucleotides encoding a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 8.

In some of any embodiments, the NiV-F protein has the sequence set forth in SEQ ID NO:23 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 23. In some of any embodiments, the F protein comprises the sequence set forth in SEQ ID NO:23 and the G protein comprises the sequence set forth in SEQ ID NO:16. In some of any embodiments, the F protein consists or consists essentially of the sequence set forth in SEQ ID NO:23 and the G protein consists or consists essentially of the sequence set forth in SEQ ID NO:16.

Provided herein is a vector, comprising the polynucleotide of any of the embodiments described herein. In some of any embodiments, the vector is a mammalian vector, viral vector or artificial chromosome, optionally wherein the artificial chromosome is a bacterial artificial chromosome (BAC).

Provided herein is a plasmid, comprising the polynucleotide of any of the embodiments described herein. In some of any embodiments, the plasmid further comprises one or more nucleic acids encoding proteins for lentivirus production.

Provided herein is a cell comprising the polynucleotide of any of embodiments described herein or the vector of any of the embodiments described herein, or the plasmid of any of the embodiments described herein.

Provided herein is a method of making a targeted lipid particle comprising a henipavirus F protein molecule or biologically active portion thereof and a targeted envelope protein comprising a henipavirus envelope attachment glycoprotein G (G protein) or a biologically active portion thereof and a single domain antibody (sdAb) variable domain, the method comprising a) providing a cell that comprises a nucleic acid encoding a henipavirus F protein molecule or biologically active portion thereof and a nucleic acid encoding a targeted envelope protein, the targeted envelope protein comprising a henipavirus envelope attachment glycoprotein G (G protein) or a biologically active portion thereof and a single domain antibody (sdAb) variable domain; b) culturing the cell under conditions that allow for production of a targeted lipid particle, and c) separating, enriching, or purifying the targeted lipid particle from the cell, thereby making the targeted lipid particle.

Provided herein is a method of making a pseudotyped lentiviral vector, the method comprising a) providing a producer cell that comprises a lentiviral viral nucleic acid(s), a nucleic acid encoding a henipavirus F protein molecule or biologically active portion thereof, and a nucleic acid encoding a targeted envelope protein, said targeted envelope protein comprising a henipavirus envelope attachment glycoprotein G (G protein) or a biologically active portion thereof and a single domain antibody; b) culturing the cell under conditions that allow for production of the lentiviral vector, and c) separating, enriching, or purifying the lentiviral vector from the cell, thereby making the pseudotyped lentiviral vector.

In some of any embodiments, the single domain antibody binds a cell surface molecule present on a target cell. In some of any embodiments, the cell surface molecule is a protein, glycan, lipid or low molecular weight molecule. In some of any embodiments, the target cell is selected from the group consisting of tumor-infiltrating lymphocytes, T cells, neoplastic or tumor cells, virus-infected cells, stem cells, central nervous system (CNS) cells, hematopoeietic stem cells (HSCs), liver cells or fully differentiated cells. In some of any embodiments, the target cell is selected from the group consisting of a CD3+ T cell, a CD4+ Tcell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell. In some of any embodiments, the single domain antibody binds an antigen or portion thereof present on a target cell.

Provided herein is a method of making a targeted lipid particle comprising a henipavirus F protein molecule or biologically active portion thereof and a targeted envelope protein comprising a henipavirus envelope attachment glycoprotein G (G protein) or a biologically active portion thereof and a binding domain, the method comprising a) providing a cell that comprises a nucleic acid encoding a henipavirus F protein molecule or biologically active portion thereof and a nucleic acid encoding a targeted envelope protein, the targeted envelope protein comprising a henipavirus envelope attachment glycoprotein G (G protein) or a biologically active portion thereof and binding domain, wherein the binding domain (i) binds a cell surface molecule selected from the group consisting of ASGR1, ASGR2, and TM4SF5, optionally human ASGR1, human ASGR2 and human ASGR2; (ii) binds a cell surface molecule selected from the group consisting of CD4 or CD8, optionally human CD4 or human CD8; or (iii) binds a cell surface molecule that is low density lipoprotein receptor (LDL-R), optionally human LDL-R; b) culturing the cell under conditions that allow for production of a targeted lipid particle, and c) separating, enriching, or purifying the targeted lipid particle from the cell, thereby making the targeted lipid particle.

Provided herein is a method of making a pseudotyped lentiviral vector, the method comprising a) providing a producer cell that comprises a lentiviral viral nucleic acid(s), a nucleic acid encoding a henipavirus F protein molecule or biologically active portion thereof, and a nucleic acid encoding a targeted envelope protein, said targeted envelope protein comprising a henipavirus envelope attachment glycoprotein G (G protein) or a biologically active portion thereof and binding domain, wherein the binding domain: (i) binds a cell surface molecule selected from the group consisting of ASGR1, ASGR2, and TM4SF5, optionally human ASGR1, human ASGR2 and human ASGR2; (ii) binds a cell surface molecule selected from the group consisting of CD4 or CD8, optionally human CD4 or human CD8; or (iii) binds a cell surface molecule that is low density lipoprotein receptor (LDL-R), optionally human LDL-R; b) culturing the producer cell under conditions that allow for production of a lentiviral vector, and c) separating, enriching, or purifying the lentiviral vector from the cell, thereby making the pseudotyped lentiviral vector.

In some of any embodiments, the binding domain is a single domain antibody. In some of any embodiments, the binding domain is a single chain variable fragment (scFv). In some of any embodiments, the cell surface molecule is selected from the group consisting of ASGR1, ASGR2 and TM4SF5. In some of any embodiments, the cell surface molecule is CD8 or CD4, In some of any embodiments, the cell surface molecule is LDL-R.

Provided herein is a method of making a targeted lipid particle comprising a henipavirus F protein molecule or biologically active portion thereof and a targeted envelope protein comprising a) providing a cell that comprises the polynucleotide of any of the embodiments provided herein the vector of any of the embodiments described herein, or the plasmid of any of the embodiments described herein; b) culturing the cell under conditions that allow for production of a targeted lipid particle, and c) separating, enriching, or purifying the targeted lipid particle particle from the cell, thereby making the targeted lipid particle.

Provided herein is a method of making a pseudotyped lentiviral vector, comprising: a) providing a producer cell that comprises a lentiviral viral nucleic acid(s), and the polynucleotide of any of the embodiments listed herein or the vector of any of the embodiments listed herein b) culturing the cell under conditions that allow for production of the lentiviral vector, and c) separating, enriching, or purifying the lentiviral vector from the cell, thereby making the pseudotyped lentiviral vector. In some of any embodiments, prior to step (b) the method further comprises providing the cell a polynucleotide encoding a henipavirus F protein molecule or biologically active portion thereof.

In some of any embodiments, the cell is a mammalian cell.

In some of any embodiments, the cell is a producer cell comprising viral nucleic acid. In some of any embodiments, the viral nucleic acid is a retroviral nucleic acid or lentiviral nucleic acid and the targeted lipid particle is a viral particle or a viral-like particle. In some of any embodiments, the viral particle or a viral-like particle is a retroviral particle or a retroviral-like particle. In some embodiments, the viral particle or a viral-like particle is a lentiviral particle or lentiviral-like particle.

In some of any embodiments, the viral nucleic acid(s) lacks one or more genes involved in viral replication. In some of any embodiments, the viral nucleic acid comprises a nucleic acid encoding a viral packaging protein selected from one or more of Gag, Pol, Rev and Tat. In some of any embodiments, the viral nucleic acid comprises:one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

Provided herein is a producer cell comprising the polynucleotide of any of the embodiments listed herein or the vector of any of the embodiments listed herein, or the plasmid of any of the embodiments described herein.

In some of any embodiments, the producer cell further comprises a nucleic acid encoding a henipavirus F protein or a biologically active portion thereof.

In some of any embodiments, the cell further comprises a viral nucleic acid. In some of any embodiments, the viral nucleic acid is a lentiviral nucleic acid. Provided herein is a producer cell comprising (i) a viral nucleic acid(s) and (ii) nucleic acid encoding a henipavirus F protein molecule or biologically active portion thereof and (iii) a nucleic acid encoding a targeted envelope protein comprising a henipavirus envelope attachment glycoprotein G (G protein) or a biologically active portion thereof and a single domain antibody (sdAb) variable domain, optionally wherein the viral nucleic acid(s) are lentiviral nucleic acids. In some of any embodiments the single domain antibody binds a cell surface molecule present on a target cell. In some of any embodiments the cell surface molecule is a protein, glycan, lipid or low molecular weight molecule.

In some of any embodiments the target cell is selected from the group consisting of tumor-infiltrating lymphocytes, T cells, neoplastic or tumor cells, virus-infected cells, stem cells, central nervous system (CNS) cells, hematopoeietic stem cells (HSCs), liver cells or fully differentiated cells. In some of any embodiments the target cell is selected from the group consisting of a CD3+ T cell, a CD4+ Tcell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell. In some of any embodiments the single domain antibody binds an antigen or portion thereof present on a target cell.

Provided herein is a producer cell comprising (i) a viral nucleic acid(s) and (ii) nucleic acid encoding a henipavirus F protein molecule or biologically active portion thereof and (iii) a nucleic acid encoding a targeted envelope protein comprising a henipavirus envelope attachment glycoprotein G (G protein) or a biologically active portion thereof and binding domain, wherein the binding domain (i) binds a cell surface molecule selected from the group consisting of ASGR1, ASGR2, and TM4SF5, optionally human ASGR1, human ASGR2 and human ASGR2; (ii) binds a cell surface molecule selected from the group consisting of CD4 or CD8, optionally human CD4 or human CD8; or (iii) binds a cell surface molecule that is low density lipoprotein receptor (LDL-R), optionally human LDL-R. In some of any embodiments the viral nucleic acid(s) are lentiviral nucleic acid.

In some of any embodiments the cell surface molecule or antigen is selected from the group consisting of ASGR1, ASGR2 and TM4SF5. In some of any embodiments, the cell surface molecule or antigen is CD8 or CD4. In some of any embodiments, the cell surface molecule or antigen is LDL-R.

In some of any embodiments, the viral nucleic acid(s) lacks one or more genes involved in viral replication. In some of any embodiments, the viral nucleic acid comprises a nucleic acid encoding a viral packaging protein selected from one or more of Gag, Pol, Rev and Tat.

In some of any embodiments, the viral nucleic acid comprises one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

In some of any embodiments, the henipavirus F protein molecule or biologically active portion thereof comprises: (i) the sequence set forth in SEQ ID NO: 2; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:2. In some of any embodiments, the henipavirus F protein molecule or biologically active portion thereof comprises (i) the sequence set forth in SEQ ID NO: 5; (ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:5.

In some of any embodiments, the henipavirus F protein molecule or biologically active portion thereof comprises (i) the sequence set forth in SEQ ID NO: 7; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:7. In some of any embodiments, the henipavirus F protein molecule or biologically active portion thereof comprises (i) a sequence encoding by a nucleotide sequence encoding the sequence set forth in SEQ ID NO: 8; (ii) a amino acid sequence encoded by a nucleotide sequence encoding a sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:8.

In some of any embodiments, the henipavirus F protein molecule or biologically active portion thereof comprises: (i) the sequence set forth in SEQ ID NO: 23; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:23.

In some of any embodiments, the henipavirus envelope attachment glycoprotein G (G protein) or a biologically active portion thereof comprises: (i) the sequence set forth in SEQ ID NO:9, SEQ ID NO:28 or SEQ ID NO:44; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:9, SEQ ID NO:28 or SEQ ID NO:44.

In some of any embodiments, the henipavirus envelope attachment glycoprotein G (G protein) or a biologically active portion thereof comprises: (i) the sequence set forth in SEQ ID NO: 10; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:10.

In some of any embodiments, the henipavirus envelope attachment glycoprotein G (G protein) or a biologically active portion thereof comprises: (i) the sequence set forth in SEQ ID NO: 35; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:35.

In some of any embodiments, the henipavirus envelope attachment glycoprotein G (G protein) or a biologically active portion thereof comprises: (i) the sequence set forth in SEQ ID NO: 45; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:45.

In some of any embodiments, the henipavirus envelope attachment glycoprotein G (G protein) or a biologically active portion thereof comprises: (i) the sequence set forth in SEQ ID NO: 11; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:11.

In some of any embodiments, the henipavirus envelope attachment glycoprotein G (G protein) or a biologically active portion thereof comprises: (i) the sequence set forth in SEQ ID NO: 36; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:36.

In some of any embodiments, the henipavirus envelope attachment glycoprotein G (G protein) or a biologically active portion thereof comprises: (i) the sequence set forth in SEQ ID NO: 46; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:46.

In some of any embodiments, the henipavirus envelope attachment glycoprotein G (G protein) or a biologically active portion thereof comprises: (i) the sequence set forth in SEQ ID NO: 12; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:12.

In some of any embodiments, the henipavirus envelope attachment glycoprotein G (G protein) or a biologically active portion thereof comprises: (i) the sequence set forth in SEQ ID NO: 37; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:37.

In some of any embodiments, the henipavirus envelope attachment glycoprotein G (G protein) or a biologically active portion thereof comprises: (i) the sequence set forth in SEQ ID NO: 47; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:47.

In some of any embodiments, the henipavirus envelope attachment glycoprotein G (G protein) or a biologically active portion thereof comprises (i) the sequence set forth in SEQ ID NO: 13; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:13.

In some of any embodiments, the henipavirus envelope attachment glycoprotein G (G protein) or a biologically active portion thereof comprises: (i) the sequence set forth in SEQ ID NO: 38; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:38.

In some of any embodiments, the henipavirus envelope attachment glycoprotein G (G protein) or a biologically active portion thereof comprises: (i) the sequence set forth in SEQ ID NO: 48; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:48.

In some of any embodiments, the henipavirus envelope attachment glycoprotein G (G protein) or a biologically active portion thereof comprises (i) the sequence set forth in SEQ ID NO: 14; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:14.

In some of any embodiments, the henipavirus envelope attachment glycoprotein G (G protein) or a biologically active portion thereof comprises: (i) the sequence set forth in SEQ ID NO: 39; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:39.

In some of any embodiments, the henipavirus envelope attachment glycoprotein G (G protein) or a biologically active portion thereof comprises: (i) the sequence set forth in SEQ ID NO: 49; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:49.

In some of any embodiments, the henipavirus envelope attachment glycoprotein G (G protein) or a biologically active portion thereof comprises (i) the sequence set forth in SEQ ID NO: 15; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:15.

In some of any embodiments, the henipavirus envelope attachment glycoprotein G (G protein) or a biologically active portion thereof comprises: (i) the sequence set forth in SEQ ID NO: 40; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:40.

In some of any embodiments, the henipavirus envelope attachment glycoprotein G (G protein) or a biologically active portion thereof comprises: (i) the sequence set forth in SEQ ID NO: 50; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:50.

In some of any embodiments, the henipavirus envelope attachment glycoprotein G (G protein) or a biologically active portion thereof comprises (i) the sequence set forth in SEQ ID NO: 16; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:16.

In some of any embodiments, the henipavirus envelope attachment glycoprotein G (G protein) or a biologically active portion thereof comprises (i) the sequence set forth in SEQ ID NO: 51; (ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:51.

In some aspects of the provided embodiments, the targeted lipid particle has greater expression of the targeted envelope protein compared to a reference lipid particle that has incorporated into a similar lipid bilayer the same envelope protein but that is fused to an alternative targeting moiety, optionally wherein the alternative targeting moiety is a single chain variable fragment (scFv). In some of any embodiments, the expression is increased by at or greater than 5%, 10%, 20%, 30%, 40%, 50%, 60%, 70%, 80%, 90%, 100%, 125%, 150%, 200%, 300%, 400%, 500% or more. In some embodiments, the expression is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, preferably at or about or greater than 10-fold or more. In some of any embodiments, the titer in target cells following transduction is at or greater than 1×10⁶ transduction units (TU)/mL, at or greater than 2×10⁶ TU/mL, at or greater than 3×10⁶ TU/mL, at or greater than 4×10⁶ TU/mL, at or greater than 5×10⁶ TU/mL, at or greater than 6×10⁶ TU/mL, at or greater than 7×10⁶ TU/mL, at or greater than 8×10⁶ TU/mL, at or greater than 9×10⁶ TU/mL, or at or greater than 1×10⁷ TU/mL. Also provided herein is a composition wherein among the population of lipid particles, greater than at or about 50%, greater than at or about 55%, greater than at or about 60%, greater than at or about 65%, greater than at or about 70%, or greater than at or about 75% are surface positive for the targeted envelope protein. In some of any embodiments, the targeted envelope protein is present on the surface of the targeted lipid particle at a density of at least about (0.001, 0.002, 0.005, 0.01, 0.02, 0.05, 0.1, 0.2 or 0.5) targeted envelope proteins/nm².

Provided herein is a viral vector particle or viral-like particle produced from the producer cell of any of the embodiments provided herein.

Provided herein is a composition comprising a plurality of targeted lipid particles of any of the embodiments provided herein. In some embodiments, the composition further includes a pharmaceutically acceptable carrier. In some of any embodiments, the targeted lipid particles comprise an average diameter of less than 1 In some of any embodiments, the composition further includes a targeted envelope protein present on the surface of the targeted lipid particles at an average density of at least about (0.001, 0.002, 0.005, 0.01, 0.02, 0.05, 0.1, 0.2 or 0.5) targeted envelope proteins/nm².

Provided herein is a producer cell containing greater membrane (e.g., plasma membrane) expression of the targeted envelope protein compared to a reference producer cell that has incorporated into its membrane (e.g. plasma membrane) the same envelope protein but that is fused to an alternative targeting moiety, optionally wherein the alternative targeting moiety is a single chain variable fragment (scFv). In some embodiments, the expression is increased by at or greater than 5%, 10%, 20%, 30%, 40%, 50%, 60%, 70%, 80%, 90%, 100%, 125%, 150%, 200%, 300%, 400%, 500% or more. In some embodiments, the expression is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, preferably at or about or greater than 10-fold or more. In some embodiments, the producer cell has the expression of the targeted envelope protein on a membrane (e.g., plasma membrane) of the producer cell is at least 20 proteins (e.g., at least 50, 100, 200, 500, 1000, 2000, 5000, or 10,000 proteins) per square micron. In some of any embodiments, the targeted envelope protein comprises at least 0.1% (e.g., at least 0.2%, 0.5%, 1%, 2%, 3%, 4%, 5%, 6%, 7%, 8%, 9%, or 10%) of the total membrane (e.g., plasma membrane) proteins of the producer cell (e.g., by total protein weight).

Provided herein is a method of transducing a cell comprising transducing a cell with any of the viral vectors described herein or with any of the compositions described herein. In some of any embodiments, the targeted envelope protein of the lentiviral vector or targeted lipid particle targets CD4 and the cell is a CD4+ cell. In some of any embodiments, the targeted envelope protein of the lentiviral vector targets CD8 and the cell is a CD8+ cell. In some of any embodiments, the targeted envelope protein of the lentiviral vector targets ASGR1, ASGR2 or TM4SF5 and the cell is a hepatocyte.

Provided herein is a method of delivering an exogenous agent to a subject (e.g., a human subject), the method comprising administering to the subject the targeted lipid particle of any of the embodiments provided herein or the composition of any of the embodiments provided herein, wherein the targeted lipid particle or lentiviral vector comprise the exogenous agent.

Provided herein is a method of delivering an exogenous agent to a subject (e.g., a human subject), the method comprising administering to the subject any of the compositions described herein, wherein targeted lipid particle or lentiviral vectors of the plurality comprise the exogenous agent.

Provided herein is a method of delivering a chimeric antigen receptor (CAR) to a cell, comprising contacting a cell with any of the lentiviral vectors described herein or a targeted lipid particle of any of the embodiments described herein, wherein the lentiviral vector or targeted lipid particle comprise nucleic acid encoding the CAR.

Provided herein is a method of delivering a chimeric antigen receptor (CAR) to a cell, comprising contacting a cell with any of the compositions described herein, wherein lentiviral vectors or targeted lipid particles of the plurality comprise nucleic acid encoding the CAR.

Provided herein is a method of delivering an exogenous agent to a hepatocyte, comprising contacting a cell with any of the lentiviral vectors described herein, or a targeted lipid particle or lentiviral vector of any of the embodiments described herein.

Provided herein is a method of delivering an exogenous agent to a hepatocyte, comprising contacting a cell with any of the compositions described herein, wherein lentiviral vectors or targeted lipid particles of the plurality comprise an exogenous agent for delivery to the hepatocyte. In some of any embodiments, the contacting transduces the cell with lentiviral vector or the targeted lipid particle.

Provided herein is a method of treating a disease or disorder in a subject (e.g., a human subject), the method comprising administering to the subject the targeted lipid particle of any of the embodiments provided herein or the composition of any of the embodiments provided herein.

Provided herein is a method of fusing a mammalian cell to a targeted lipid particle, the method comprising administering to the subject the targeted lipid particle of any of the embodiments provided herein or the composition of any of the embodiments provided herein. In some of any embodiments, the fusing of the mammalian cell to the targeted lipid particle delivers an exogenous agent to a subject (e.g., a human subject). In some of any embodiments, the fusing of the mammalian cell to the targeted lipid particle treats a disease or disorder in a subject (e.g., a human subject). In some of any embodiments, the targeted envelope protein of the lentiviral vector or targeted lipid particle targets CD4 and the cell is a CD4+ cell. In some of any embodiments, the targeted envelope protein of the lentiviral vector targets CD8 and the cell is a CD8+ cell. In some of any embodiments, the targeted envelope protein of the lentiviral vector targets ASGR1, ASGR2 or TM4SF5 and the cell is a hepatocyte.

In some of any embodiments, the targeted lipid particle has greater expression of the targeted envelope protein compared to a reference lipid particle that has incorporated into a similar lipid bilayer the same envelope protein but that is fused to an alternative targeting moiety. In some embodiments, the alternative targeting moiety is a single chain variable fragment (scFv). In some of any embodiments, the expression is increased by at or greater than 5%, 10%, 20%, 30%, 40%, 50%, 60%, 70%, 80%, 90%, 100%, 125%, 150%, 200%, 300%, 400%, 500% or more. In some of any embodiments, the expression is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, preferably at or about or greater than 10-fold or more.

In some of any embodiments, the titer in target cells following transduction is at or greater than 1×10⁶ transduction units (TU)/mL, at or greater than 2×10⁶ TU/mL, at or greater than 3×10⁶ TU/mL, at or greater than 4×10⁶ TU/mL, at or greater than 5×10⁶ TU/mL, at or greater than 6×10⁶ TU/mL, at or greater than 7×10⁶ TU/mL, at or greater than 8×10⁶ TU/mL, at or greater than 9×10⁶ TU/mL, or at or greater than 1×10⁷ TU/mL.

In some of any embodiments, among the population of lipid particles or lentiviral vectors in the composition, greater than at or about 50%, greater than at or about 55%, greater than at or about 60%, greater than at or about 65%, greater than at or about 70%, or greater than at or about 75% are surface positive for the targeted envelope protein. In some of any embodiments, the targeted envelope protein is present on the surface of the targeted lipid particle at a density of at least about (0.001, 0.002, 0.005, 0.01, 0.02, 0.05, 0.1, 0.2 or 0.5) targeted envelope proteins/nm².

Provided herein is a composition comprising a plurality of the targeted lipid particles of any of the embodiments described herein or a plurality of lentiviral vectors of any of the embodiments described herein, wherein the targeted envelope protein is present on the surface of the targeted lipid particles at an average density of at least about (0.001, 0.002, 0.005, 0.01, 0.02, 0.05, 0.1, 0.2 or 0.5) targeted envelope proteins/nm².

In some of any embodiments, the producer cell has greater membrane (e.g., plasma membrane) expression of the targeted envelope protein compared to a reference producer cell that has incorporated into its membrane (e.g. plasma membrane) the same envelope protein but that is fused to an alternative targeting moiety, optionally wherein the alternative targeting moiety is a single chain variable fragment (scFv). In some of any embodiments, the expression is increased by at or greater than 5%, 10%, 20%, 30%, 40%, 50%, 60%, 70%, 80%, 90%, 100%, 125%, 150%, 200%, 300%, 400%, 500% or more. In some of any embodiments, the expression is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, preferably at or about or greater than 10-fold or more. In some of any embodiments, the producer cell has the expression of the targeted envelope protein on a membrane (e.g., plasma membrane) of the producer cell is at least 20 proteins (e.g., at least 50, 100, 200, 500, 1000, 2000, 5000, or 10,000 proteins) per square micron. In some of any embodiments, the targeted envelope protein comprises at least 0.1% (e.g., at least 0.2%, 0.5%, 1%, 2%, 3%, 4%, 5%, 6%, 7%, 8%, 9%, or 10%) of the total membrane (e.g., plasma membrane) proteins of the producer cell (e.g., by total protein weight).

### DETAILED DESCRIPTION

Provided herein are targeted lipid particles containing a lipid bilayer enclosing a lumen or cavity and a targeted envelope protein containing (1) a henipavirus envelope attachment glycoprotein G (G protein) or biologically active portion thereof and (2) a binding domain, such as a a single domain antibody (sdAb) variable domain, in which the targeted envelope protein is embedded in the lipid bilayer of the lipid particles. In particular embodiments, the binding domain, such as a single domain antibody, is an antibody with the ability to bind, such as specifically bind, to a desired target molecule. Exemplary binding domains are described in Section II.A.2. In some embodiments, the targeted lipid particles also contains a henipavirus fusion (F) protein molecule or a biologically active portion thereof embedded in the lipid bilayer. In particular embodiments, the lipid particles can be a virus-like particle, a virus, or a viral vector, such as a lentiviral vector.

In some embodiments, one or both of the G protein and the F protein is from a Hendra (HeV) or a Nipah (NiV) virus, or is a biologically active portion thereof or is a variant or mutant thereof. In particular embodiments, both the G protein and the F protein is from a Hendra (HeV) or a Nipah (NiV) virus. In some embodiments, the fusion and attachment glycoproteins mediate cellular entry of Nipah virus.

The F protein, such as NiV-F, is a class I fusion protein that has structural and functional features in common with fusion proteins of many families (e.g., HIV-1 gp41 or influenza virus hemagglutinin [HA]), such as an ectodomain with a hydrophobic fusion peptide and two heptad repeat regions (White JM et al. 2008. Crit Rev Biochem Mol Biol 43:189-219). F proteins are synthesized as inactive precursors F₀ and are activated by proteolytic cleavage into the two disulfide-linked subunits F₁ and F₂ (Moll M. et al. 2004. J. Virol. 78(18): 9705-9712).

G proteins are attachment proteins of henipavirus (e.g. Nipah virus or Hendra virus) that are type II transmembrane glycoproteins containing an N-terminal cytoplasmic tail, a transmembrane domain, an extracellular stalk, and a globular head (Liu, Q. et al. 2015. Journal of Virology, 89(3):1838-1850). The attachment protein, NiV-G, recognizes the receptors EphrinB2 and EphrinB3. Binding of the receptor to NiV-G triggers a series of conformational changes that eventually lead to the triggering of NiV-F, which exposes the fusion peptide of NiV-F, allowing another series of conformational changes that lead to virus-cell membrane fusion (Stone J. A. et al. 2016. J Virol. 90(23): 10762-10773). EphrinB2 was previously identified as the primary NiV receptor (Negrete et al., 2005), as well as EphrinB3 as an alternate receptor (Negrete et al., 2006). In fact, NiV-G has a high affinity for EphrinB2 and B3, with affinity binding constants (Kd) in the picomolar range (Negrete et al., 2006) (Kd=0.06 nM and 0.58 nM for cell surface expressed ephrinB2 and B3, respectively).

The efficiency of transduction of targeted lipid particles can be improved by engineering hyperfusogenic mutations in one or both of NiV-F and NiV-G. Several such mutations have been previously described (see, e.g., Lee at al, 2011, Trends in Microbiology). This could be useful, for example, for maintaining the specificity and picomolar affinity of NiV-G for EphrinB2 and/or B3. Additionally, mutations in NiV-G that completely abrogate EphrinB2 and B3 binding, but that do not impact the association of this NiV-G with NiV-F, have been identified. Methods to improve targeting of lipid particles can be achieved by fusion of a binding molecule with a G protein (e.g. Niv-G, including a Niv-G with mutations to abrogate ephrin B2 and ephrin B3 binding). This could allow for altered G protein tropism allowing for targeting of other desired cell types that are not EphrinB2+ through the addition of the binding molecule molecule directed against a different cell surface molecule.

While retargeted lipid particles incorporating such binding molecules fused to a G protein have been generated, it is found herein that some some binding molecules when fused with a G protein (e.g. NiV-G) express better on the surface of lipid particles than others. For example, it is found that single domain antibodies (sdAbs), such as VHH, may express 10-fold better than a single chain variable fragment (scFv). Without wishing to be bound by theory, the increase in expression may be due to an increased stability of the retargeted G protein on the surface of the lipid particle. This greater expression can improve the ability of the lipid particle to target the target molecule (e.g. a cell surface molecule) compared to a similar lipid particle but containing an alternative binding domain, e.g. scFv, against the same target molecule.

Thus, provided herein are targeted lipid particles containing a G protein of a henipavirus (e.g. Hendra or Nipah, e.g. NiV-G) attached to a sdAb variable domain directed against or that is able to bind to a cell surface molecule on a target cell. sdAb variable domains can include those of a VL or VH only sdAb, nanobodies, camelid VHH domains, shark IgNAR or fragments thereof. In some embodiments, the sdAb is a VHH.

In aspects of the provided embodiments, a targeted lipid particle can be engineered to express a henipavirus F protein molecule or biologically active portion thereof; and a targeted envelope protein comprising (i) a henipavirus envelope attachment glycoprotein G (G protein) or a biologically active portion thereof and (ii) single domain antibody (sdAb) variable domain, wherein the F protein molecule or the biologically active portion thereof and the targeted envelope protein are embedded in the lipid bilayer. In some embodiments, the sdAb variable domain is attached to the C-terminus of the G protein or the biologically active portion thereof. In some embodiments, the sdAb variable domain is attached to the G protein via a linker.

Also provided are targeted lipid particles additionally containing one or more exogenous agents, such as for delivery of a diagnostic or therapeutic agent to cells, including following in vivo administration to a subject. Also provided herein are methods and uses of the targeted lipid particles, such in diagnostic and therapeutic methods. Also provided are polynucleotides, methods for engineering, preparing, and producing the targeted lipid non-cell particles, compositions containing the particles, and kits and devices containing and for using, producing and administering the particles.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### BRIEF DESCRIPTION OF THE DRAWINGS

FIGS. 1A-1C depict characterization of cells transfected with constructs containing scFv or VHH binding modalities. FIG. 1A depicts surface expression of cells transfected with constructs containing scFV or VHH binding modalities, analyzed by flow cytometry, and depicted as median fluorescence intensity (MFI), quantified by % of His+ cells. FIG. 1B depicts binding to soluble hCD4-Fc protein of cells transfected with constructs containing scFV of VHH binding modalities analyzed by flow cytometry, and depicted as median fluorescence intensity (MFI), quantified by % Fc+ cell. FIG. 1C depicts surface expression of targeted binding sequences on 293 cells for cells transfected with constructs containing VHH binding modalities, compared to the scFv binding modalities, analyzed by flow cytometry, and depicted as median fluorescence intensity (MFI), as quantified by % of His+ cells. Empty vector and the expression vector without the binder domain were used as negative controls.

FIG. 2 depicts transduction efficacy of four exemplary constructs containing scFV or VHH binding modalities on PanT cells from peripheral blood that were negatively selected to enrich for T cells were thawed and activated with anti CD3/anti-CD28. Cells were analyzed by flow cytometry, and titer determined by % of CD4-positive cells that were GFP+.

FIGS. 3A-3B depict transduction efficiency of CD8 retargeted pseudotyped lentiviruses in an in vivo model using activated PBMCs injected intraperitonally into NOD-scid-IL2rγ^{null} mice, as analyzed by flow cytometry. Transduciton efficiency of CD8 retargeted pseudotyped lentiviruses is depicted on CD8+ (FIG. 3A) or CD8− (FIG. 3B) T cells, and titer was determined by % of CD8 positive or negative cells that were GFP+.

FIGS. 4A-4B depict the ability of CD8 retargeted pseudotyped lentiviruses containing chimeric antigen receptors (CARs) to effect killing of leukemic cells in vitro. FIG. 4A shows the ability to detect CD19+ CAR expression on CD8+ cells at 4 days post transduction. FIG. 4B shows the elimination of Nalm6 cells evaluated at 18 hours post incubation, analyzed by flow cytometry

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Unless defined otherwise, all technical and scientific terms, acronyms, and abbreviations used herein have the same meaning as commonly understood by one of ordinary skill in the art to which the invention pertains. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, “lipid particle” refers to any biological or synthetic particle that contains a bilayer of amphipathic lipids enclosing a lumen or cavity. Typically a lipid particle does not contain a nucleus. Examples of lipid particles include solid particles such as nanoparticles, viral-derived particles or cell-derived particles. Such lipid particles include, but are not limited to, viral particles (e.g. lentiviral particles), virus-like particles, viral vectors (e.g., lentiviral vectors) exosomes, enucleated cells, various vesicles, such as a microvesicle, a membrane vesicle, an extracellular membrane vesicle, a plasma membrane vesicle, a giant plasma membrane vesicle, an apoptotic body, a mitoparticle, a pyrenocyte, or a lysosome. In some embodiments, a lipid particle can be a fusosome. In some embodiments, the lipid particle is not a platelet.

As used herein a “biologically active portion,” such as with reference to a protein such as a G protein or an F protein, refers to a portion of the protein that exhibits or retains an activity or property of the full-length of the protein. For example, a biologically active portion of an F protein retains fusogenic activity in conjunction with the G protein when each are embedded in a lipid bilayer. A biologically active portion of the G protein retains fusogenic activity in conjunction with an F protein when each is embedded in a lipid bilayer. The retained activity and include 10%-150% or more of the activity of a full-length or wild-type F protein or G protein. Examples of biologically active portions of F and G proteins include truncations of the cytoplasmic domain, e.g. truncations of up to 1, 2, 3, 4, 5, 6, 7, 8 9, 10, 11, 12, 13, 14, 15, 20, 25, 30, 35 or more contiguous amino acids, see e.g. Khetawat and Broder 2010 Virology Journal 7:312; Witting et al. 2013 Gene Therapy 20:997-1005; published international; patent application No. WO/2013/148327.

As used herein, “fusosome” refers to a particle containing a bilayer of amphipathic lipids enclosing a lumen or cavity and a fusogen that interacts with the amphipathic lipid bilayer. In embodiments, the fusosome comprises a nucleic acid. In some embodiments, the fusosome is a membrane enclosed preparation. In some embodiments, the fusosome is derived from a source cell.

As used herein, “fusosome composition” refers to a composition comprising one or more fusosomes.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain.

As used herein, a “re-targeted fusogen” refers to a fusogen that comprises a targeting moiety having a sequence that is not part of the naturally-occurring form of the fusogen. In embodiments, the fusogen comprises a different targeting moiety relative to the targeting moiety in the naturally-occurring form of the fusogen. In embodiments, the naturally-occurring form of the fusogen lacks a targeting domain, and the re-targeted fusogen comprises a targeting moiety that is absent from the naturally-occurring form of the fusogen. In embodiments, the fusogen is modified to comprise a targeting moiety. In embodiments, the fusogen comprises one or more sequence alterations outside of the targeting moiety relative to the naturally-occurring form of the fusogen, e.g., in a transmembrane domain, fusogenically active domain, or cytoplasmic domain.

As used herein, a “targeted envelope protein” refers to a polypeptide that contains a henipavirus G protein attached to a single domain antibody (sdAb) variable domain, such as a VL or VH only sdAb, nanobodies, camelid VHH domains, shark IgNAR or fragments thereof, that targets a molecule on a desired cell type. In some such embodiments, the attachment may be directly or indirectly via a linker, such as a peptide linker.

As used herein, a “targeted lipid particle” refers to a lipid particle that contains a targeted envelope protein embedded in the lipid bilayer.

As used herein, a “retroviral nucleic acid” refers to a nucleic acid containing at least the minimal sequence requirements for packaging into a retrovirus or retroviral vector, alone or in combination with a helper cell, helper virus, or helper plasmid. In some embodiments, the retroviral nucleic acid further comprises or encodes an exogenous agent, a positive target cell-specific regulatory element, a non-target cell-specific regulatory element, or a negative TCSRE. In some embodiments, the retroviral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (Ψ), RRE (e.g., to bind to Rev and promote nuclear export). The retroviral nucleic acid can comprise RNA (e.g., when part of a virion) or DNA (e.g., when being introduced into a source cell or after reverse transcription in a recipient cell). In some embodiments, the retroviral nucleic acid is packaged using a helper cell, helper virus, or helper plasmid which comprises one or more of (e.g., all of) gag, pol, and env.

As used herein, a “target cell” refers to a cell of a type to which it is desired that a targeted lipid particle delivers an exogenous agent. In embodiments, a target cell is a cell of a specific tissue type or class, e.g., an immune effector cell, e.g., a T cell. In some embodiments, a target cell is a diseased cell, e.g., a cancer cell. In some embodiments, the fusogen, e.g., re-targeted fusogen leads to preferential delivery of the exogenous agent to a target cell compared to a non-target cell.

As used herein a “non-target cell” refers to a cell of a type to which it is not desired that a targeted lipid particle delivers an exogenous agent. In some embodiments, a non-target cell is a cell of a specific tissue type or class. In some embodiments, a non-target cell is a non-diseased cell, e.g., a non-cancerous cell. In some embodiments, the fusogen, e.g., re-targeted fusogen leads to lower delivery of the exogenous agent to a non-target cell compared to a target cell.

As used herein, a “single domain antibody” or “sdAb” refers to an antibody having a single monomeric domain antigen binding/recognition domain. Such antibodies include nanobodies, camelid antibodies (e.g. VHH), or shark antibodies (e.g. IgNAR). In some embodiments, a variable domain of a sdAb comprises three CDRs and four framework regions, designated FR1, CDR1, FR2, CDR2, FR3, CDR3, and FR4. In some embodiments, a sdAb variable domain may be truncated at the N-terminus or C-terminus such that it comprise only a partial FR1 and/or FR4, or lacks one or both of those framework regions, so long as the sdAb variable domain substantially maintains antigen binding and specificity.

The term “CDR” denotes a complementarity determining region as defined by at least one manner of identification to one of skill in the art. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

In some embodiments, CDRs can be defined in accordance with any of the Chothia numbering schemes, the Kabat numbering scheme, a combination of Kabat and Chothia, the AbM definition, and/or the contact definition. A sdAb variable domain comprises three CDRs, designated CDR1, CDR2, and CDR3. Table 1, below, lists exemplary position boundaries of CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-H1 located before CDR-H1, FR-H2 located between CDR-H1 and CDR-H2, FR-H3 located between CDR-H2 and CDR-H3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-H1 | H31--H35B | H26--H32 . . . 34 | H26--H35B | H30--H35B |
| (Kabat | | | | |
| Num- | | | | |
| bering¹) | | | | |
| CDR-H1 | H31--H35 | H26--H32 | H26--H35 | H30--H35 |
| (Chothia | | | | |
| Num- | | | | |
| bering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given sdAb amino acid sequence, it is understood that such a CDR has a sequence of the corresponding CDR (e.g., CDR-H3) within the sdAb, as defined by any of the aforementioned schemes. It is understood that any antibody, such as a sdAb, includes CDRs and such can be identified according to any of the other aforementioned numbering schemes or other numbering schemes known to a skilled artisan.

As used herein, the term “specifically binds” to a target molecule, such as an antigen, means that a binding molecule, such as a single domain antibody, reacts or associates more frequently, more rapidly, with greater duration and/or with greater affinity with a particular target molecule than it does with alternative molecules. A binding molecule, such as a sdAb variable domain, “specifically binds” to a target molecule if it binds with greater affinity, avidity, more readily, and/or with greater duration than it binds to other molecules. It is understood that a binding molecule, such as a sdAb, that specifically binds to a first target may or may not specifically bind to a second target. As such, “specific binding” does not necessarily require (although it can include) exclusive binding.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 2 Amino acid substitutions may be introduced into an antibody of interest and the products screened for a desired activity, for example, retained/improved binding.

TABLE 2

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The term “effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a targeted lipid particle, refers to an agent that is neither comprised by nor encoded in the corresponding wild-type virus or fusogen made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

As used herein, a “promoter” refers to a cis-regulatory DNA sequence that, when operably linked to a gene coding sequence, drives transcription of the gene. The promoter may comprise a transcription factor binding sites. In some embodiments, a promoter works in concert with one or more enhancers which are distal to the gene.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutically acceptable” refers to a material, such as carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical. composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

A “disease” or “disorder” as used herein refers to a condition where treatment is needed and/or desired.

As used herein, the terms “treat,” “treating,” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder can include obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

The terms “individual” and “subject” are used interchangeably herein to refer to an animal; for example a mammal. The term patient includes human and veterinary subjects. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder. In particular embodiments, the subject is a human, such as a human patient.

### II. TARGETED LIPID PARTICLES (E.G. LENTIVIRAL VECTORS)

Provided herein are targeted lipid particles that comprise a henipavirus F protein molecule or biologically active portion thereof, and a targeted envelope protein comprising (i) a henipavirus envelope attachment glycoprotein G (G protein) or a biologically active portion thereof and (ii) binding domain, wherein the binding domain is attached to the C-terminus of the G protein or the biologically active portion, wherein each of (i) and (ii) is exposed on the outer surface of the targeted lipid particle. In some embodiments, the binding domain is a single domain antibody. In some embodiments, the binding domain is a single chain variable fragment. In particular embodiments, the provided lipid particles exhibit fusogenic activity, which is mediated by the targeted envelope protein that facilitates binding to a target cell and contains the G protein or biologically active portion thereof, and the F glycoprotein that is involved in facilitating the merger or fusion of the two lumens of the lipid particle and the target cell membranes.

Provided herein are targeted lipid particles that comprise a henipavirus F protein molecule or biologically active portion thereof, and a targeted envelope protein comprising (i) a henipavirus envelope attachment glycoprotein G (G protein) or a biologically active portion thereof and (ii) a single domain antibody (sdAb) variable domain, wherein the single domain antibody is attached to the C-terminus of the G protein or the biologically active portion, wherein each of (i) and (ii) is exposed on the outer surface of the targeted lipid particle. In particular embodiments, the provided lipid particles exhibit fusogenic activity, which is mediated by the targeted envelope protein that facilitates binding to a target cell and contains the G protein or biologically active portion thereof, and the F glycoprotein that is involved in facilitating the merger or fusion of the two lumens of the lipid particle and the target cell membranes.

In some of any embodiment, the targeted lipid particles are viral particles or viral-like particles. In some aspects, such targeted lipid particles contain viral nucleic acid, such as retroviral nucleic acid, for example lentiviral nucleic acid. In particular embodiments, any provided targeted lipid particles, such as a viral particle or viral-like particle, is replication defective. In some embodiments, the targeted lipid particle is a lentiviral vector, in which the lentiviral vector is pseudotyped with the henipavirus F protein and the targeted envelope protein.

For instance, provided herein is a pseudotyped lentiviral vector that comprises a henipavirus F protein molecule or biologically active portion thereof, and a targeted envelope protein comprising (i) a henipavirus envelope attachment glycoprotein G (G protein) or a biologically active portion thereof and (ii) binding domain, wherein the binding domain is attached to the C-terminus of the G protein or the biologically active portion, wherein each of (i) and (ii) is exposed on the outer surface of the targeted lipid particle. In some embodiments, the binding domain is a single domain antibody. In some embodiments, the binding domain is a single chain variable fragment.

In some embodiments, the targeted lipid particle provided herein (e.g. targeted lentiviral vector) has increased or greater expression of the targeted envelope protein compared to a reference lipid particle (e.g. reference lentiviral vector) that incorporates a similar envelope protein but that is fused to an alternative targeting moiety other than a sdAb variable domain, such as a single chain variable fragment (scFv). In some embodiments, such targeted lipid particles are produced by pseudotyping of lipid particles (e.g lentiviral particles) following co-transfection of the packaging cells with the transfer, envelope, and gag-pol plasmids.

In some embodiments, the expression is increased by at or greater than 5%, 10%, 20%, 30%, 40%, 50%, 60%, 70%, 80%, 90%, 100%, 125%, 150%, 200%, 300%, 400%, 500% or more, compared to a reference lipid particle (e.g. reference lentiviral vector), e.g. a reference lipid particle containing a similar envelope protein but that is fused to an scFv. In some examples, the expression is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, compared to a reference lipid particle (e.g. reference lentiviral vector), e.g. a reference lipid particle containing a similar envelope protein but that is fused to an scFv. In some embodiments, expression can be assayed in vitro using flow cytometry, e.g. FACs. In some embodiments, expression can be depicted as the number or density of targeted envelope protein on the surface of a targeted lipid particle (e.g. targeted lentiviral vector). In some embodiments, expression can be depicted as the mean fluorescent intensity (MFI) of surface expression of the targeted envelope protein on the surface of a targeted lipid particle (e.g. targeted lentiviral vector). In some embodiments, expression can be depicted as the percent of lipid particle (e.g. lentiviral vectors) in a population that are surface positive for the targeted envelope protein.

In some embodiments, in a population of targeted lipid particles (e.g. targeted lentiviral vectors) greater than at or about 50% of the lipid particles are surface positive for the targeted envelope protein. For example, in a population of provided targeted lipid particles (e.g. targeted lentiviral vectors) greater than at or about 55%, greater than at or about 60%, greater than at or about 65%, greater than at or about 70%, greater than at or about 75% of the cells in the population are surface positive for the targeted envelope protein.

In some embodiments, titer of the targeted lipid particles following introduction into target cells, such as by transduction (e.g. transduced cells), is increased compared to titer into the same target cells of reference lipid particles (e.g. reference lentiviral vector) that incorporate a similar envelope protein but fused to an alternative targeting moiety other than a sdAb variable domain, such as a single chain variable fragment (scFv). Typically, the alternative targeting moiety recognizes or binds the same target molecule as the sdAb variable domain of the targeted envelope protein of the targeted lipid particles. In some embodiments, the titer is increased by at or greater than 5%, 10%, 20%, 30%, 40%, 50%, 60%, 70%, 80%, 90%, 100%, 125%, 150%, 200%, 300%, 400%, 500% or more, compared to titer of a reference lipid particle (e.g. reference lentiviral vector), e.g. a reference lipid particle containing a similar envelope protein but that is fused to an scFv. In some examples, the titer is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, compared to the titer of a reference lipid particle (e.g. reference lentiviral vector), e.g. a reference lipid particle containing a similar envelope protein but that is fused to an scFv. In some embodiments, the titer of the targeted lipid particles in target cells (e.g. transduced cells) is greater than at or about 1×10⁶ transduction units (TU)/mL. For example, the titer of the targeted lipid particles in target cells (e.g. transduced cells) is greater than at or about 2×10⁶ TU/mL, greater than at or about 3×10⁶ TU/mL, greater than at or about 4×10⁶ TU/mL, greater than at or about 5×10⁶ TU/mL, greater than at or about 6×10⁶ TU/mL, greater than at or about 7×10⁶ TU/mL, greater than at or about 8×10⁶ TU/mL, greater than at or about 9×10⁶ TU/mL, or greater than at or about 1×10⁷ TU/mL.

A. Targeted Envelope Protein (e.g. Henipavirus Plus Binding Domain)

In some embodiments, the targeted lipid particle (e.g. lentiviral vector) includes a targeted envelope protein exposed on the surface of the targeted lipid particle (e.g. lentiviral vector).

In some embodiments, the targeted envelope protein contains a henipavirus envelope attachment glycoprotein G (G protein) or a biologically active portion thereof and a binding domain that binds to a cell surface molecule on a target cell. In some embodiments, the binding domain is a single domain antibody (sdAb). In some embodiments, the binding domain is a single chain variable fragment (scFv). The binding domain can be linked directly or indirectly to the G protein. In particular embodiments, the binding domain is linked to the C-terminus (C-terminal amino acid) of the G protein or the biologically active portion thereof. The linkage can be via a peptide linker, such as a flexible peptide linker.

I. Protein

In some embodiments, the targeted envelope protein contains a henipavirus envelope attachment glycoprotein G (G protein) or a biologically active portion thereof and a single domain antibody (sdAb) variable domain or biologically active portion thereof. In some embodiments, the sdAb binds to a cell surface molecule on a target cell. The sdAb variable domain can be linked directly or indirectly to the G protein. In particular embodiments, the sdAb variable domain is linked to the C-terminus (C-terminal amino acid) of the G protein or the biologically active portion thereof. The linkage can be via a peptide linker, such as a flexible peptide linker.

In some embodiments, an binding domain (e.g. sdAb) binds to a cell surface antigen of a cell. In some embodiments, a cell surface antigen is characteristic of one type of cell. In some embodiments, a cell surface antigen is characteristic of more than one type of cell.

In some embodiments, the binding domain (e.g. sdAb) variable domain binds a cell surface molecule or antigen. In some embodiments, the cell surface molecule is ASGR1, ASGR2, TM4SF5, CD8, CD4, or low density lipoprotein receptor (LDL-R). In some embodiments, the cell surface molecule is ASGR1. In some embodiments, the cell surface molecule is ASGR2. In some embodiments, the cell surface molecule is TM4SF5. In some embodiments, the cell surface molecule is CD8. In some embodiments, the cell surface molecule is CD4. In some embodiments, the cell surface molecule is LDL-R.

In some embodiments the G protein is a Henipavirus G protein or a biologically active portion thereof. In some embodiments, the Henipavirus G protein is a Hendra (HeV) virus G protein, a Nipah (NiV) virus G-protein (NiV-G), a Cedar (CedPV) virus G-protein, a Mojiang virus G-protein, a bat Paramyxovirus G-protein or a biologically active portion thereof. Table 3 provides non-limiting examples of G proteins.

The attachment G proteins are type II transmembrane glycoproteins containing an N-terminal cytoplasmic tail (e.g. corresponding to amino acids 1-49 of SEQ ID NO:9), a transmembrane domain (e.g. corresponding to amino acids 50-70 of SEQ ID NO:9), and an extracellular domain containing an extracellular stalk (e.g. corresponding to amino acids 71-187 of SEQ ID NO:9), and a globular head (corresponding to amino acids 188-602 of SEQ ID NO:9). The N-terminal cytoplasmic domain is within the inner lumen of the lipid bilayer and the C-terminal portion is the extracellular domain that is exposed on the outside of the lipid bilayer. Regions of the stalk in the C-terminal region (e.g. corresponding to amino acids 159-167 of NiV-G) have been shown to be involved in interactions with F protein and triggering of F protein fusion (Liu et al. 2015 J of Virology 89:1838). In wild-type G protein, the globular head mediates receptor binding to henipavirus entry receptors eprhin B2 and ephrin B3, but is dispensable for membrane fusion (Brandel-Tretheway et al. Journal of Virology. 2019. 93(13)e00577-19). In particular embodiments herein, tropism of the G protein is altered by linkage of the G protein or biologically active fragment thereof (e.g. cytoplasmic truncation) to a sdAb variable domain. Binding of the G protein to a binding partner can trigger fusion mediated by a compatible F protein or biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acids identity. Studies have shown a high degree of compatibility among G proteins with F proteins of different species as demonstrated by heterotypic fusion activation (Brandel-Tretheway et al. Journal of Virology. 2019). As described further below, a re-targeted lipid particle can contain heterologous G and F proteins from different species.

TABLE 3
Henipavirus protein G sequence clusters. Column 1, Genbank ID includes the
Genbank ID of the whole genome sequence of the virus that is the centroid sequence of the
cluster. Column 2, nucleotides of CDS provides the nucleotides corresponding to the CDS of
the gene in the whole genome. Column 3, Full Gene Name, provides the full name of the gene
including Genbank ID, virus species, strain, and protein name. Column 4, Sequence, provides
the amino acid sequence of the gene. Column 5, #Sequences/Cluster, provides the number of
sequences that cluster with this centroid sequence. Column 6 provides the SEQ ID numbers for
the described sequences.

| | | | | | | SEQ |
|---|---|---|---|---|---|---|
| | | | | | | ID |
| | | | | | | NO |
| | | | | | | (without |
| | Nucleotides | | | | SEQ | N- |
| Genbank | of | Full sequence | | #Sequences/ | ID | terminal |
| ID | CDS | ID | Sequence | Cluster | NO | methionine) |
| AF017 | 8913- | gb: AF017149\| | MMADSKLVSLNNNLSGKIKDQGKVIKN | 14 | 18 | 52 |
| 149 | 10727 | Organism: Hen | YYGTMDIKKINDGLLDSKILGAFNTVIA | | | |
| | | dra | LLGSIIIIVMNIMIIQNYTRTTDNQALIKES | | | |
| | | virus\|Strain | LQSVQQQIKALTDKIGFEIGPKVSLIDTSS | | | |
| | | Name: UNKN | TITIPANIGLLGSKISQSTSSINENVNDKC | | | |
| | | OWN- | KFTLPPLKIHECNISCPNPLPFREYRPISQ | | | |
| | | AF017149\|Pro | GVSDLVGLPNQICLQKTTSTILKPRLISY | | | |
| | | tein | TLPINTREGVCITDPLLAVDNGFFAYSHL | | | |
| | | Name: glycopr | EKIGSCTRGIAKQRIIGVGEVLDRGDKVP | | | |
| | | otein\|Gene | SMFMTNVWTPPNPSTIHHCSSTYHEDFY | | | |
| | | Symbol: G | YTLCAVSHVGDPILNSTSWTESLSLIRLA | | | |
| | | | VRPKSDSGDYNQKYIAITKVERGKYDK | | | |
| | | | VMPYGPSGIKQGDTLYFPAVGFLPRTEF | | | |
| | | | QYNDSNCPIIHCKYSKAENCRLSMGVNS | | | |
| | | | KSHYILRSGLLKYNLSLGGDIILQFIEIAD | | | |
| | | | NRLTIGSPSKIYNSLGQPVFYQASYSWD | | | |
| | | | TMIKLGDVDTVDPLRVQWRNNSVISRP | | | |
| | | | GQSQCPRFNVCPEVCWEGTYNDAFLIDR | | | |
| | | | LNWVSAGVYLNSNQTAENPVFAVFKDN | | | |
| | | | EILYQVPLAEDDTNAQKTITDCFLLENVI | | | |
| | | | WCISLVEIYDTGDSVIRPKLFAVKIPAQC | | | |
| | | | SES | | | |
| AF212 | 8943- | gb: AF2123021 | MPAENKKVRFENTTSDKGKIPSKVIKSY | 14 | 28 | 44 |
| 302 | 10751 | Organism: Nip | YGTMDIKKINEGLLDSKILSAFNTVIALL | | | |
| | | ah virus\|Strain | GSIVIIVMNIMIIQNYTRSTDNQAVIKDA | | | |
| | | Name: UNKN | LQGIQQQIKGLADKIGTEIGPKVSLIDTSS | | | |
| | | OWN- | TITIPANIGLLGSKISQSTASINENVNEKC | | | |
| | | AF212302\|Pro | KFTLPPLKIHECNISCPNPLPFREYRPQTE | | | |
| | | tein | GVSNLVGLPNNICLQKTSNQILKPKLISY | | | |
| | | Name: attachm | TLPVVGQSGTCITDPLLAMDEGYFAYSH | | | |
| | | ent | LERIGSCSRGVSKQRIIGVGEVLDRGDEV | | | |
| | | glycoprotein\|G | PSLFMTNVWTPPNPNTVYHCSAVYNNE | | | |
| | | ene Symbol: G | FYYVLCAVSTVGDPILNSTYWSGSLMM | | | |
| | | | TRLAVKPKSNGGGYNQHQLALRSIEKG | | | |
| | | | RYDKVMPYGPSGIKQGDTLYFPAVGFL | | | |
| | | | VRTEFKYNDSNCPITKCQYSKPENCRLS | | | |
| | | | MGIRPNSHYILRSGLLKYNLSDGENPKV | | | |
| | | | VFIEISDQRLSIGSPSKIYDSLGQPVFYQA | | | |
| | | | SFSWDTMIKFGDVLTVNPLVVNWRNNT | | | |
| | | | VISRPGQSQCPRFNTCPEICWEGVYNDA | | | |
| | | | FLIDRINWISAGVFLDSNQTAENPVFTVF | | | |
| | | | KDNEILYRAQLASEDTNAQKTITNCFLL | | | |
| | | | KNKIWCISLVEIYDTGDNVIRPKLFAVKI | | | |
| | | | PEQCT | | | |
| JQ001 | 8170- | gb: JQ001776: | MLSQLQKNYLDNSNQQGDKMNNPDKK | 3 | 29 | 54 |
| 776 | 10275 | 8170- | LSVNFNPLELDKGQKDLNKSYYVKNKN | | | |
| | | 10275\|Organis | YNVSNLLNESLHDIKFCIYCIFSLLIIITIIN | | | |
| | | m: Cedar | IITISIVITRLKVHEENNGMESPNLQSIQD | | | |
| | | virus\|S train | SLSSLTNMINTEITPRIGILVTATSVTLSSS | | | |
| | | Name: CG1a\|Pr | INYVGTKTNQLVNELKDYITKSCGFKVP | | | |
| | | otein | ELKLHECNISCADPKISKSAMYSTNAYA | | | |
| | | Name: attachm | ELAGPPKIFCKSVSKDPDFRLKQIDYVIP | | | |
| | | ent | VQQDRSICMNNPLLDISDGFFTYIHYEGI | | | |
| | | glycoprotein\|G | NSCKKSDSFKVLLSHGEIVDRGDYRPSL | | | |
| | | ene Symbol: G | YLLSSHYHPYSMQVINCVPVTCNQSSFV | | | |
| | | | FCHISNNTKTLDNSDYSSDEYYITYFNGI | | | |
| | | | DRPKTKKIPINNMTADNRYIHFTFSGGG | | | |
| | | | GVCLGEEFIIPVTTVINTDVFTHDYCESF | | | |
| | | | NCSVQTGKSLKEICSESLRSPTNSSRYNL | | | |
| | | | NGIMIISQNNMTDFKIQLNGITYNKLSFG | | | |
| | | | SPGRLSKTLGQVLYYQSSMSWDTYLKA | | | |
| | | | GFVEKWKPFTPNWMNNTVISRPNQGNC | | | |
| | | | PRYHKCPEICYGGTYNDIAPLDLGKDMY | | | |
| | | | VSVILDSDQLAENPEITVFNSTTILYKER | | | |
| | | | VSKDELNTRSTTTSCFLFLDEPWCISVLE | | | |
| | | | TNRFNGKSIRPEIYSYKIPKYC | | | |
| NC_02 | 9117- | gb: NC_02525 | MPQKTVEFINMNSPLERGVSTLSDKKTL | 2 | 30 | 55 |
| 5256 | 11015 | 6: 9117- | NQSKITKQGYFGLGSHSERNWKKQKNQ | | | |
| | | 11015\|Organis | NDHYMTVSTMILEILVVLGIMFNLIVLT | | | |
| | | m: Bat | MVYYQNDNINQRMAELTSNITVLNLNL | | | |
| | | Paramyxovirus | NQLTNKIQREIIPRITLIDTATTITIPSAITY | | | |
| | | Eid_he1/GH- | ILATLTTRISELLPSINQKCEFKTPTLVLN | | | |
| | | M74a/GHA/20 | DCRINCTPPLNPSDGVKMSSLATNLVAH | | | |
| | | 09\|Strain | GPSPCRNFSSVPTIYYYRIPGLYNRTALD | | | |
| | | Name: BatPV/ | ERCILNPRLTISSTKFAYVHSEYDKNCTR | | | |
| | | Eid_he1/GH- | GFKYYELMTFGEILEGPEKEPRMFSRSF | | | |
| | | M74a/GHA/20 | YSPTNAVNYHSCTPIVTVNEGYFLCLEC | | | |
| | | 09\|Protein | TSSDPLYKANLSNSTFHLVILRHNKDEKI | | | |
| | | Name: glycopr | VSMPSFNLSTDQEYVQIIPAEGGGTAESG | | | |
| | | otein\|Gene | NLYFPCIGRLLHKRVTHPLCKKSNCSRT | | | |
| | | Symbol: G | DDESCLKSYYNQGSPQHQVVNCLIRIRN | | | |
| | | | AQRDNPTWDVITVDLTNTYPGSRSRIFG | | | |
| | | | SFSKPMLYQSSVSWHTLLQVAEITDLDK | | | |
| | | | YQLDWLDTPYISRPGGSECPFGNYCPTV | | | |
| | | | CWEGTYNDVYSLTPNNDLFVTVYLKSE | | | |
| | | | QVAENPYFAIFSRDQILKEFPLDAWISSA | | | |
| | | | RTTTISCFMFNNEIWCIAALEITRLNDDII | | | |
| | | | RPIYYSFWLPTDCRTPYPHTGKMTRVPL | | | |
| | | | RSTYNY | | | |
| NC_02 | 8716- | gb: NC_02535 | MATNRDNTITSAEVSQEDKVKKYYGVE | 2 | 31 | 56 |
| 5352 | 11257 | 2: 8716- | TAEKVADSISGNKVFILMNTLLILTGAIIT | | | |
| | | 11257\|Organis | ITLNITNLTAAKSQQNMLKIIQDDVNAK | | | |
| | | m: Mojiang | LEMFVNLDQLVKGEIKPKVSLINTAVSV | | | |
| | | virus\|Strain | SIPGQISNLQTKFLQKYVYLEESITKQCT | | | |
| | | Name: Tonggu | CNPLSGIFPTSGPTYPPTDKPDDDTTDDD | | | |
| | | an1\|Protein | KVDTTIKPIEYPKPDGCNRTGDHFTMEP | | | |
| | | Name: attachm | GANFYTVPNLGPASSNSDECYTNPSFSIG | | | |
| | | ent | SSIYMFSQEIRKTDCTAGEILSIQIVLGRI | | | |
| | | glycoprotein\|G | VDKGQQGPQASPLLVWAVPNPKIINSCA | | | |
| | | ene Symbol: G | VAAGDEMGWVLCSVTLTAASGEPIPHM | | | |
| | | | FDGFWLYKLEPDTEVVSYRITGYAYLLD | | | |
| | | | KQYDSVFIGKGGGIQKGNDLYFQMYGL | | | |
| | | | SRNRQSFKALCEHGSCLGTGGGGYQVL | | | |
| | | | CDRAVMSFGSEESLITNAYLKVNDLASG | | | |
| | | | KPVIIGQTFPPSDSYKGSNGRMYTIGDKY | | | |
| | | | GLYLAPSSWNRYLRFGITPDISVRSTTWL | | | |
| | | | KSQDPIMKILSTCTNTDRDMCPEICNTRG | | | |
| | | | YQDIFPLSEDSEYYTYIGITPNNGGTKNF | | | |
| | | | VAVRDSDGHIASIDILQNYYSITSATISCF | | | |
| | | | MYKDEIWCIAITEGKKQKDNPQRIYAHS | | | |
| | | | YKIRQMCYNMKSATVTVGNAKNITIRR | | | |
| | | | Y | | | |

In some embodiments, the G protein has a sequence set forth in any of SEQ ID NOS: 9, 18, 28, 29, 30, 31, 44, 52, or 54-56 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 9, 18, 28, 29, 30, 31, 44, 52, or 54-56. In particular embodiments, the G protein or functionally active variant or biologically active portion is a protein that retains fusogenic activity in conjunction with a Henipavirus F protein, such as an F protein set forth in Section I.B (e.g. NiV-F or HeV-F). Fusogenic activity includes the activity of the G protein in conjunction with a Henipavirus F protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F).

In particular embodiments, the G protein has the sequence of amino acids set forth in SEQ ID NO: 9, SEQ ID NO: 28, SEQ ID NO: 18, SEQ ID NO: 30, SEQ ID NO: 31, SEQ ID NO: 44, SEQ ID NO: 52 or SEQ ID NO: 54-56 or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:9, SEQ ID NO:28, SEQ ID NO: 18, SEQ ID NO:30, SEQ ID NO: 31, SEQ ID NO: 44, SEQ ID NO: 52 or SEQ ID NO: 54-56 and retains fusogenic activity in conjunction with a Henipavirus F protein (e.g., NiV-F or HeV-F). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:9, SEQ ID NO:28, SEQ ID NO: 18, SEQ ID NO:30 SEQ ID NO: 31, SEQ ID NO: 44, SEQ ID NO: 52 or SEQ ID NO: 54-56 and retains fusogenic activity in conjunction with a Henipavirus F protein (e.g., NiV-F or HeV-F).

Reference to retaining fusogenic activity includes activity (in conjunction with a Henipavirus F protein) that is between at or about 10% and at or about 150% or more of the level or degree of binding of the corresponding wild-type G protein, such as set forth in SEQ ID NO:9, SEQ ID NO:28, SEQ ID NO: 18, SEQ ID NO:30, SEQ ID NO: 31, SEQ ID NO: 44, SEQ ID NO: 52 or SEQ ID NO: 54-56 such as at least or at least about 10% of the level or degree of fusogenic activity of the corresponding wild-type G protein, such as at least or at least about 15% of the level or degree of fusogenic activity of the corresponding wild-type G protein, such as at least or at least about 20% of the level or degree of fusogenic activity of the corresponding wild-type G protein, such as at least or at least about 25% of the level or degree of fusogenic activity of the corresponding wild-type G protein, such as at least or at least about 30% of the level or degree of fusogenic activity of the corresponding wild-type G protein, such as at least or at least about 35% of the level or degree of fusogenic activity of the corresponding wild-type G protein, such as at least or at least about 40% of the level or degree of fusogenic activity of the corresponding wild-type G protein, such as at least or at least about 45% of the level or degree of fusogenic activity of the corresponding wild-type G protein, such as at least or at least about 50% of the level or degree of fusogenic activity of the corresponding wild-type G protein, such as at least or at least about 55% of the level or degree of fusogenic activity of the corresponding wild-type G protein, such as at least or at least about 60% of the level or degree of fusogenic activity of the corresponding wild-type G protein, such as at least or at least about 65% of the level or degree of fusogenic activity of the corresponding wild-type G protein, such as at least or at least about 70% of the level or degree of fusogenic activity of the corresponding wild-type G protein, such as at least or at least about 75% of the level or degree of fusogenic activity of the corresponding wild-type G protein, such as at least or at least about 80% of the level or degree of fusogenic activity of the corresponding wild-type G protein, such as at least or at least about 85% of the level or degree of fusogenic activity of the corresponding wild-type G protein, such as at least or at least about 90% of the level or degree of fusogenic activity of the corresponding wild-type G protein, such as at least or at least about 95% of the level or degree of fusogenic activity of the corresponding wild-type G protein, such as at least or at least about 100% of the level or degree of fusogenic activity of the corresponding wild-type G protein, or such as at least or at least about 120% of the level or degree of fusogenic activity of the corresponding wild-type G protein.

In some embodiments the G protein is a mutant G protein that is a functionally active variant or biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein or biologically active portion thereof. In some embodiments, the wild-type G protein has the sequence set forth in any one of SEQ ID NOS: 9, 18, 28, 29, 30, 31 SEQ ID NO: 44, SEQ ID NO: 52 or SEQ ID NO: 54-56.

In some embodiments, the G protein is a mutant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein. In particular embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NOS: 9, 18, 28, 29, 30, 31, SEQ ID NO: 44, SEQ ID NO: 52 or SEQ ID NO: 54-56. In some embodiments, the mutant F protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, the G protein is a wild-type Nipah virus G (NiV-G) protein or a Hendra virus G protein, or is a functionally active variant or biologically active portion thereof. In some embodiments, the G protein is a NiV-G protein that has the sequence set forth in SEQ ID NO:9, SEQ ID NO:28 or SEQ ID NO:44, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:9, SEQ ID NO:28 or SEQ ID NO:44.

In some embodiments, the G protein is a mutant NiV-G protein that is a biologically active portion of a wild-type NiV-G. In some embodiments, the biologically active portion is an N-terminally truncated fragment. In some embodiments, the mutant NiV-G protein is truncated and lacks up to 5 contiguous amino acid residues at or near the N-terminus of the wild-type NiV-G protein (SEQ ID NO:9, SEQ ID NO:28 or SEQ ID NO:44), up to 6 contiguous amino acid residues at or near the N-terminus of the wild-type NiV-G protein (SEQ ID NO:9, SEQ ID NO:28 or SEQ ID NO:44), up to 7 contiguous amino acid residues at or near the N-terminus of the wild-type NiV-G protein (SEQ ID NO:9, SEQ ID NO:28 or SEQ ID NO:44), up to 8 contiguous amino acid residues at or near the N-terminus of the wild-type NiV-G protein (SEQ ID NO:9, SEQ ID NO:28 or SEQ ID NO:44), up to 9 contiguous amino acid residues at or near the N-terminus of the wild-type NiV-G protein (SEQ ID NO:9, SEQ ID NO:28 or SEQ ID NO:44), up to 10 contiguous amino acid residues at or near the N-terminus of the wild-type NiV-G protein (SEQ ID NO:9, SEQ ID NO:28 or SEQ ID NO:44), up to 11 contiguous amino acid residues at or near the N-terminus of the wild-type NiV-G protein (SEQ ID NO:9, SEQ ID NO:28 or SEQ ID NO:44), up to 12 contiguous amino acid residues at or near the N-terminus of the wild-type NiV-G protein (SEQ ID NO:9, SEQ ID NO:28 or SEQ ID NO:44), up to 13 contiguous amino acid residues at or near the N-terminus of the wild-type NiV-G protein (SEQ ID NO:9, SEQ ID NO:28 or SEQ ID NO:44), up to 14 contiguous amino acid residues at or near the N-terminus of the wild-type NiV-G protein (SEQ ID NO:9, SEQ ID NO:28 or SEQ ID NO:44), up to 15 contiguous amino acid residues at or near the N-terminus of the wild-type NiV-G protein (SEQ ID NO:9, SEQ ID NO:28 or SEQ ID NO:44), up to 16 contiguous amino acid residues at or near the N-terminus of the wild-type NiV-G protein (SEQ ID NO:9, SEQ ID NO:28 or SEQ ID NO:44), up to 17 contiguous amino acid residues at or near the N-terminus of the wild-type NiV-G protein (SEQ ID NO:9, SEQ ID NO:28 or SEQ ID NO:44), up to 18 contiguous amino acid residues at or near the N-terminus of the wild-type NiV-G protein (SEQ ID NO:9, SEQ ID NO:28 or SEQ ID NO:44), up to 19 contiguous amino acid residues at or near the N-terminus of the wild-type NiV-G protein (SEQ ID NO:9, SEQ ID NO:28 or SEQ ID NO:44), up to 20 contiguous amino acid residues at or near the N-terminus of the wild-type NiV-G protein (SEQ ID NO:9, SEQ ID NO:28 or SEQ ID NO:44), up to 21 contiguous amino acid residues at or near the N-terminus of the wild-type NiV-G protein (SEQ ID NO:9, SEQ ID NO:28 or SEQ ID NO:44), up to 22 contiguous amino acid residues at or near the N-terminus of the wild-type NiV-G protein (SEQ ID NO:9, SEQ ID NO:28 or SEQ ID NO:44), up to 23 contiguous amino acid residues at or near the N-terminus of the wild-type NiV-G protein (SEQ ID NO:9, SEQ ID NO:28 or SEQ ID NO:44), up to 24 contiguous amino acid residues at or near the N-terminus of the wild-type NiV-G protein (SEQ ID NO:9, SEQ ID NO:28 or SEQ ID NO:44), up to 25 contiguous amino acid residues at or near the N-terminus of the wild-type NiV-G protein (SEQ ID NO:9, SEQ ID NO:28 or SEQ ID NO:44), up to 26 contiguous amino acid residues at or near the N-terminus of the wild-type NiV-G protein (SEQ ID NO:9, SEQ ID NO:28 or SEQ ID NO:44), up to 27 contiguous amino acid residues at or near the N-terminus of the wild-type NiV-G protein (SEQ ID NO:9, SEQ ID NO:28 or SEQ ID NO:44), up to 28 contiguous amino acid residues at or near the N-terminus of the wild-type NiV-G protein (SEQ ID NO:9, SEQ ID NO:28 or SEQ ID NO:44), up to 29 contiguous amino acid residues at or near the N-terminus of the wild-type NiV-G protein (SEQ ID NO:9, SEQ ID NO:28 or SEQ ID NO:44), up to 30 contiguous amino acid residues at or near the N-terminus of the wild-type NiV-G protein (SEQ ID NO:9, SEQ ID NO:28 or SEQ ID NO:44), up to 31 contiguous amino acid residues at or near the N-terminus of the wild-type NiV-G protein (SEQ ID NO:9, SEQ ID NO:28 or SEQ ID NO:44), up to 32 contiguous amino acid residues at or near the N-terminus of the wild-type NiV-G protein (SEQ ID NO:9, SEQ ID NO:28 or SEQ ID NO:44), up to 33 contiguous amino acid residues at or near the N-terminus of the wild-type NiV-G protein (SEQ ID NO:9, SEQ ID NO:28 or SEQ ID NO:44), up to 34 contiguous amino acid residues at or near the N-terminus of the wild-type NiV-G protein (SEQ ID NO:9, SEQ ID NO:28 or SEQ ID NO:44), up to 35 contiguous amino acid residues at or near the N-terminus of the wild-type NiV-G protein (SEQ ID NO:9, SEQ ID NO:28 or SEQ ID NO:44), up to 36 contiguous amino acid residues at or near the N-terminus of the wild-type NiV-G protein (SEQ ID NO:9, SEQ ID NO:28 or SEQ ID NO:44), up to 37 contiguous amino acid residues at or near the N-terminus of the wild-type NiV-G protein (SEQ ID NO:9, SEQ ID NO:28 or SEQ ID NO:44), up to 38 contiguous amino acid residues at or near the N-terminus of the wild-type NiV-G protein (SEQ ID NO:9, SEQ ID NO:28 or SEQ ID NO:44), up to 39 contiguous amino acid residues at or near the N-terminus of the wild-type NiV-G protein (SEQ ID NO:9, SEQ ID NO:28 or SEQ ID NO:44), up to 40 contiguous amino acid residues at or near the N-terminus of the wild-type NiV-G protein (SEQ ID NO:9, SEQ ID NO:28 or SEQ ID NO:44), up to 41 contiguous amino acid residues at or near the N-terminus of the wild-type NiV-G protein (SEQ ID NO:9, SEQ ID NO:28 or SEQ ID NO:44), up to 42 contiguous amino acid residues at or near the N-terminus of the wild-type NiV-G protein (SEQ ID NO:9, SEQ ID NO:28 or SEQ ID NO:44), up to 43 contiguous amino acid residues at or near the N-terminus of the wild-type NiV-G protein (SEQ ID NO:9, SEQ ID NO:28 or SEQ ID NO:44), up to 44 contiguous amino acid residues at or near the N-terminus of the wild-type NiV-G protein (SEQ ID NO:9, SEQ ID NO:28 or SEQ ID NO:44), or up to 45 contiguous amino acid residues at or near the N-terminus of the wild-type NiV-G protein (SEQ ID NO:9, SEQ ID NO:28 or SEQ ID NO:44).

In some embodiments, the NiV-G protein is a biologically active portion that does not contain a cytoplasmic domain. In some embodiments, the NiV-G protein without the cytoplasmic domain is encoded by SEQ ID NO: 32.

In some embodiments, the mutant NiV-G protein comprises a sequence set forth in any of SEQ ID NOS: 10-15, 35-40, 45-50, 22, 53 or SEQ ID NO: 32, or is a functional variant thereof that has an amino acid sequence having at least at or 80%, at least at or about 81%, at least at or about 82%, at least at or about 83%, at or about 84%, at least at or about 85%, at least at or about 86%, or at least at or about 87%, at least at or about 88%, or at least at or about 89%, about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs: 10-15, 35-40, 45-50, 22, 53 or SEQ ID NO:32.

In some embodiments, the mutant NiV-G protein has a 5 amino acid truncation at or near the N-terminus of the wild-type NiV-G protein (SEQ ID NO:9, SEQ ID NO:28 or SEQ ID NO:44), such as set forth in SEQ ID NO: 10 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:10 or such as set forth in SEQ ID NO: 35 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:35 or such as set forth in SEQ ID NO: 45 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:45. In some embodiments, the mutant NiV-G protein has a 10 amino acid truncation at or near the N-terminus of the wild-type NiV-G protein (SEQ ID NO:9, SEQ ID NO:28 or SEQ ID NO:44), such as set forth in SEQ ID NO: 11 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:11, or such as set forth in SEQ ID NO: 36 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:36 or such as set forth in SEQ ID NO: 46 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46.

In some embodiments, the mutant NiV-G protein has a 15 amino acid truncation at or near the N-terminus of the wild-type NiV-G protein (SEQ ID NO:9, SEQ ID NO:28 or SEQ ID NO:44), such as set forth in SEQ ID NO: 12 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12 or such as set forth in SEQ ID NO: 37 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:37 or such as set forth in SEQ ID NO: 47 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:47. In some embodiments, the mutant NiV-G protein has a 20 amino acid truncation at or near the N-terminus of the wild-type NiV-G protein (SEQ ID NO:9, SEQ ID NO:28 or SEQ ID NO:44) such as set forth in SEQ ID NO: 13, or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:13 or such as set forth in SEQ ID NO: 38 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:38 or such as set forth in SEQ ID NO: 48 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:48. In some embodiments, the mutant NiV-G protein has a 25 amino acid truncation at or near the N-terminus of the wild-type NiV-G protein (SEQ ID NO:9, SEQ ID NO:28 or SEQ ID NO:44), such as set forth in SEQ ID NO: 14 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14 or such as set forth in SEQ ID NO: 39 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:39 or such as set forth in SEQ ID NO: 49 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:49. In some embodiments, the mutant NiV-G protein has a 30 amino acid truncation at or near the N-terminus of the wild-type NiV-G protein (SEQ ID NO:9, SEQ ID NO:28 or SEQ ID NO:44), such as set forth in SEQ ID NO: 15 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15 or such as set forth in SEQ ID NO: 40 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:40, or such as set forth in SEQ ID NO: 50 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:50. In some embodiments, the mutant NiV-G protein has a 34 amino acid truncation at or near the N-terminus of the wild-type NiV-G protein (SEQ ID NO:9, SEQ ID NO:28 or SEQ ID NO:44), such as set forth in SEQ ID NO: 22 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:22 or such as set forth in SEQ ID NO: 53 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:53. In some embodiments, the mutant NiV-G protein lacks the N-terminal cytoplasmic domain of the wild-type NiV-G protein (SEQ ID NO:9, SEQ ID NO:28 or SEQ ID NO:44), such as set forth in SEQ ID NO:32 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:32.

In some embodiments, the mutant G protein is a mutant HeV-G protein that has the sequence set forth in SEQ ID NO:18 or 52, or is a functional variant or biologically active portion thereof that has an amino acid sequence having at least at or about 80%, at least at or about 81%, at least at or about 82%, at least at or about 83%, at least at or about 84%, at or about 85%, at least at or about 86%, at least at or about 87%, at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:18 or 52.

In some embodiments, the G protein is a mutant HeV-G protein that is a biologically active portion of a wild-type HeV-G. In some embodiments, the biologically active portion is an N-terminally truncated fragment. In some embodiments, the mutant HeV-G protein is truncated and lacks up to 5 contiguous amino acid residues at or near the N-terminus of the wild-type HeV-G protein (SEQ ID NO:18 or 52), up to 6 contiguous amino acid residues at or near the N-terminus of the wild-type HeV-G protein (SEQ ID NO:18 or 52), up to 7 contiguous amino acid residues at or near the N-terminus of the wild-type HeV-G protein (SEQ ID NO:18 or 52), up to 8 contiguous amino acid residues at or near the N-terminus of the wild-type HeV-G protein (SEQ ID NO:18 or 52), up to 9 contiguous amino acid residues at or near the N-terminus of the wild-type HeV-G protein (SEQ ID NO:18 or 52), up to 10 contiguous amino acid residues at or near the N-terminus of the wild-type HeV-G protein (SEQ ID NO:18 or 52), up to 11 contiguous amino acid residues at or near the N-terminus of the wild-type HeV-G protein (SEQ ID NO:18 or 52), up to 12 contiguous amino acid residues at or near the N-terminus of the wild-type HeV-G protein (SEQ ID NO:18 or 52), up to 13 contiguous amino acid residues at or near the N-terminus of the wild-type HeV-G protein (SEQ ID NO:18 or 52), up to 14 contiguous amino acid residues at or near the N-terminus of the wild-type HeV-G protein (SEQ ID NO:18 or 52), up to 15 contiguous amino acid residues at or near the N-terminus of the wild-type HeV-G protein (SEQ ID NO:18 or 52), up to 16 contiguous amino acid residues at or near the N-terminus of the wild-type HeV-G protein (SEQ ID NO:18 or 52), up to 17 contiguous amino acid residues at or near the N-terminus of the wild-type HeV-G protein (SEQ ID NO:18 or 52), up to 18 contiguous amino acid residues at or near the N-terminus of the wild-type HeV-G protein (SEQ ID NO:18 or 52), up to 19 contiguous amino acid residues at or near the N-terminus of the wild-type HeV-G protein (SEQ ID NO:18 or 52), up to 20 contiguous amino acid residues at or near the N-terminus of the wild-type HeV-G protein (SEQ ID NO:18 or 52), up to 21 contiguous amino acid residues at or near the N-terminus of the wild-type HeV-G protein (SEQ ID NO:18 or 52), up to 22 contiguous amino acid residues at or near the N-terminus of the wild-type HeV-G protein (SEQ ID NO:18 or 52), up to 23 contiguous amino acid residues at or near the N-terminus of the wild-type HeV-G protein (SEQ ID NO:18 or 52), up to 24 contiguous amino acid residues at or near the N-terminus of the wild-type HeV-G protein (SEQ ID NO:18 or 52), up to 25 contiguous amino acid residues at or near the N-terminus of the wild-type HeV-G protein (SEQ ID NO:18 or 52), up to 26 contiguous amino acid residues at or near the N-terminus of the wild-type HeV-G protein (SEQ ID NO:18 or 52), up to 27 contiguous amino acid residues at or near the N-terminus of the wild-type HeV-G protein (SEQ ID NO:18 or 52), up to 28 contiguous amino acid residues at or near the N-terminus of the wild-type HeV-G protein (SEQ ID NO:18 or 52), up to 29 contiguous amino acid residues at or near the N-terminus of the wild-type HeV-G protein (SEQ ID NO:18 or 52), up to 30 contiguous amino acid residues at or near the N-terminus of the wild-type HeV-G protein (SEQ ID NO:18 or 52), up to 31 contiguous amino acid residues at or near the N-terminus of the wild-type HeV-G protein (SEQ ID NO:18 or 52), up to 32 contiguous amino acid residues at or near the N-terminus of the wild-type HeV-G protein (SEQ ID NO:18 or 52), up to 33 contiguous amino acid residues at or near the N-terminus of the wild-type HeV-G protein (SEQ ID NO:18 or 52), up to 34 contiguous amino acid residues at or near the N-terminus of the wild-type HeV-G protein (SEQ ID NO:18 or 52), up to 35 contiguous amino acid residues at or near the N-terminus of the wild-type HeV-G protein (SEQ ID NO:18 or 52), up to 36 contiguous amino acid residues at or near the N-terminus of the wild-type HeV-G protein (SEQ ID NO:18 or 52), up to 37 contiguous amino acid residues at or near the N-terminus of the wild-type HeV-G protein (SEQ ID NO:18 or 52), up to 38 contiguous amino acid residues at or near the N-terminus of the wild-type HeV-G protein (SEQ ID NO:18 or 52), up to 39 contiguous amino acid residues at or near the N-terminus of the wild-type HeV-G protein (SEQ ID NO:18 or 52), up to 40 contiguous amino acid residues at or near the N-terminus of the wild-type HeV-G protein (SEQ ID NO:18 or 52), up to 41 contiguous amino acid residues at or near the N-terminus of the wild-type HeV-G protein (SEQ ID NO:18 or 52), up to 42 contiguous amino acid residues at or near the N-terminus of the wild-type HeV-G protein (SEQ ID NO:18 or 52), up to 43 contiguous amino acid residues at or near the N-terminus of the wild-type HeV-G protein (SEQ ID NO:18 or 52), up to 44 contiguous amino acid residues at or near the N-terminus of the wild-type HeV-G protein (SEQ ID NO:18 or 52), or up to 45 contiguous amino acid residues at or near the N-terminus of the wild-type HeV-G protein (SEQ ID NO:18 or 52). In some embodiments, the HeV-G protein is a biologically active portion that does not contain a cytoplasmic domain. In some embodiments, the mutant HeV-G protein lacks the N-terminal cytoplasmic domain of the wild-type HeV-G protein (SEQ ID NO:18 or 52), such as set forth in SEQ ID NO:33 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:33.

In some embodiments, the G protein or the functionally active variant or biologically active portion thereof binds to Ephrin B2 or Ephrin B3. In some aspects, the G protein has the sequence of amino acids set forth in any one of SEQ ID NO:9, SEQ ID NO:18 or SEQ ID NO:28, SEQ ID NO:29, SEQ ID NO: 44, SEQ ID NO:30 or SEQ ID NO:31, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, and retains binding to Ephrhin B2 or B3. Reference to retaining binding to Ephrin B2 or B3 includes binding that is at least or at least about 5% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, 10% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, 15% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, 20% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, 25% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion, 30% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, 35% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, 40% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, 45% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, 50% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, 55% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, 60% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, 65% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, 70% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, such as at least or at least about 75% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, such as at least or at least about 80% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, such as at least or at least about 85% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, such as at least or at least about 90% of the level or degree of binding of the corresponding wild-type G protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof, or such as at least or at least about 95% of the level or degree of binding of the corresponding wild-type protein, such as set forth in SEQ ID NO:9, SEQ ID NO:18 or SEQ ID NO:28, SEQ ID NO:29, SEQ ID NO: 44, SEQ ID NO:30 or SEQ ID NO:31, or a functionally active variant or biologically active portion thereof. In some embodiments, the G protein is NiV-G or a functionally active variant or biologically active portion thereof and binds to Ephrin B2 or Ephrin B3. In some aspects, the NiV-G has the sequence of amino acids set forth in SEQ ID NO:9, SEQ ID NO:28 or SEQ ID NO:44, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:9, SEQ ID NO:28 or SEQ ID NO:44 and retains binding to Eprhin B2 or B3. Exemplary biologically active portions include N-terminally truncated variants lacking all or a portion of the cytoplasmic domain, e.g. 1 or more, such as 1 to 49 contiguous N-terminal amino acid residues, e.g. set forth in any one of SEQ ID NOS: 10-15, 35-40, 45-50 and 32. Reference to retaining binding to Ephrin B2 or B3 includes binding that is at least or at least about 5% of the level or degree of binding of the corresponding wild-type NiV-G, such as set forth in SEQ ID NO:9, SEQ ID NO:28 or SEQ ID NO:44, 10% of the level or degree of binding of the corresponding wild-type NiV-G, such as set forth in SEQ ID NO:9, SEQ ID NO:28 or SEQ ID NO:44, 15% of the level or degree of binding of the corresponding wild-type NiV-G, such as set forth in SEQ ID NO:9, SEQ ID NO:28 or SEQ ID NO:44, 20% of the level or degree of binding of the corresponding wild-type NiV-G, such as set forth in SEQ ID NO:9, SEQ ID NO:28 or SEQ ID NO:44, 25% of the level or degree of binding of the corresponding wild-type NiV-G, such as set forth in SEQ ID NO:9, SEQ ID NO:28 or SEQ ID NO:44, 30% of the level or degree of binding of the corresponding wild-type NiV-G, such as set forth in SEQ ID NO:9, SEQ ID NO:28 or SEQ ID NO:44, 35% of the level or degree of binding of the corresponding wild-type NiV-G, such as set forth in SEQ ID NO:9, SEQ ID NO:28 or SEQ ID NO:44, 40% of the level or degree of binding of the corresponding wild-type NiV-G, such as set forth in SEQ ID NO:9, SEQ ID NO:28 or SEQ ID NO:44, 45% of the level or degree of binding of the corresponding wild-type NiV-G, such as set forth in SEQ ID NO:9, SEQ ID NO:28 or SEQ ID NO:44, 50% of the level or degree of binding of the corresponding wild-type NiV-G, such as set forth in SEQ ID NO:9, SEQ ID NO:28 or SEQ ID NO:44, 55% of the level or degree of binding of the corresponding wild-type NiV-G, such as set forth in SEQ ID NO:9, SEQ ID NO:28 or SEQ ID NO:44, 60% of the level or degree of binding of the corresponding wild-type NiV-G, such as set forth in SEQ ID NO:9, SEQ ID NO:28 or SEQ ID NO:44, 65% of the level or degree of binding of the corresponding wild-type NiV-G, such as set forth in SEQ ID NO:9, SEQ ID NO:28 or SEQ ID NO:44, 70% of the level or degree of binding of the corresponding wild-type NiV-G, such as set forth in SEQ ID NO:9, SEQ ID NO:28 or SEQ ID NO:44, such as at least or at least about 75% of the level or degree of binding of the corresponding wild-type NiV-G, such as set forth in SEQ ID NO:9, SEQ ID NO:28 or SEQ ID NO:44, such as at least or at least about 80% of the level or degree of binding of the corresponding wild-type NIV-G, such as set forth in SEQ ID NO:9, SEQ ID NO:28 or SEQ ID NO:44, such as at least or at least about 85% of the level or degree of binding of the corresponding wild-type NiV-G, such as set forth in SEQ ID NO:9, SEQ ID NO:28 or SEQ ID NO:44, such as at least or at least about 90% of the level or degree of binding of the corresponding wild-type NiV-G, such as set forth in SEQ ID NO:9, SEQ ID NO:28 or SEQ ID NO:44, or such as at least or at least about 95% of the level or degree of binding of the corresponding wild-type NiV-G, such as set forth in SEQ ID NO:9, SEQ ID NO:28 or SEQ ID NO:44.

In some embodiments, the G protein is HeV-G or a functionally active variant or biologically active portion thereof and binds to Ephrin B2 or Ephrin B3. In some aspects, the HeV-G has the sequence of amino acids set forth in SEQ ID NO:18 or 52, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:18 or 52 and retains binding to Eprhin B2 or B3. Exemplary biologically active portions include N-terminally truncated variants lacking all or a portion of the cytoplasmic domain, e.g. 1 or more, such as 1 to 49 contiguous N-terminal amino acid residues, e.g. set forth in any one of SEQ ID NO:33. Reference to retaining binding to Ephrin B2 or B3 includes binding that is at least or at least about 5% of the level or degree of binding of the corresponding wild-type HeV-G, such as set forth in SEQ ID NO:18 or 52, 10% of the level or degree of binding of the corresponding wild-type HeV-G, such as set forth in SEQ ID NO:18 or 52, 15% of the level or degree of binding of the corresponding wild-type HeV-G, such as set forth in SEQ ID NO:18 or 52, 20% of the level or degree of binding of the corresponding wild-type HeV-G, such as set forth in SEQ ID NO:18 or 52, 25% of the level or degree of binding of the corresponding wild-type HeV-G, such as set forth in SEQ ID NO:18 or 52, 30% of the level or degree of binding of the corresponding wild-type HeV-G, such as set forth in SEQ ID NO:18 or 52, 35% of the level or degree of binding of the corresponding wild-type HeV-G, such as set forth in SEQ ID NO:18 or 52, 40% of the level or degree of binding of the corresponding wild-type HeV-G, such as set forth in SEQ ID NO:18 or 52, 45% of the level or degree of binding of the corresponding wild-type HeV-G, such as set forth in SEQ ID NO:18 or 52, 50% of the level or degree of binding of the corresponding wild-type HeV-G, such as set forth in SEQ ID NO:18 or 52, 55% of the level or degree of binding of the corresponding wild-type HeV-G, such as set forth in SEQ ID NO:18 or 52, 60% of the level or degree of binding of the corresponding wild-type HeV-G, such as set forth in SEQ ID NO:18 or 52, 65% of the level or degree of binding of the corresponding wild-type HeV-G, such as set forth in SEQ ID NO:18 or 52, 70% of the level or degree of binding of the corresponding wild-type HeV-G, such as set forth in SEQ ID NO:18 or 52, such as at least or at least about 75% of the level or degree of binding of the corresponding wild-type HeV-G, such as set forth in SEQ ID NO:18 or 52, such as at least or at least about 80% of the level or degree of binding of the corresponding wild-type NIV-G, such as set forth in SEQ ID NO:18 or 52, such as at least or at least about 85% of the level or degree of binding of the corresponding wild-type HeV-G, such as set forth in SEQ ID NO:18 or 52, such as at least or at least about 90% of the level or degree of binding of the corresponding wild-type HeV-G, such as set forth in SEQ ID NO:18 or 52, or such as at least or at least about 95% of the level or degree of binding of the corresponding wild-type HeV-G, such as set forth in SEQ ID NO:18 or 52.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations described herein can improve transduction efficiency. In some embodiments, the mutations described herein allow for specific targeting of other desired cell types that are not Ephrin B2 or Ephrin B3. In some embodiments, the mutations described herein result in at least the partial inability to bind at least one natural receptor, such has reduce the binding to at least one of Ephrin B2 or Ephrin B3. In some embodiments, the mutations described herein interfere with natural receptor recognition.

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO:28.

In some embodiments, the G protein is a mutant G protein containing one or more amino acid substitutions selected from the group consisting of E501A, W504A, Q530A and E533A with reference to numbering set forth in SEQ ID NO:28. In some embodiments, the G protein is a mutant G protein that contains one or more amino acid substitutions elected from the group consisting of E501A, W504A, Q530A and E533A with reference to SEQ ID NO:28 and is a biologically active portion thereof containing an N-terminal truncation. In some embodiments, the mutant NiV-G protein or the biologically active portion thereof is truncated and lacks up to 5 contiguous amino acid residues at or near the N-terminus of the wild-type NiV-G protein (SEQ ID NO:28), 6 contiguous amino acid residues at or near the N-terminus of the wild-type NiV-G protein (SEQ ID NO:28), 7 contiguous amino acid residues at or near the N-terminus of the wild-type NiV-G protein (SEQ ID NO:28), 8 contiguous amino acid residues at or near the N-terminus of the wild-type NiV-G protein (SEQ ID NO:28), 9 contiguous amino acid residues at or near the N-terminus of the wild-type NiV-G protein (SEQ ID NO:28), up to 10 contiguous amino acid residues at or near the N-terminus of the wild-type NiV-G protein (SEQ ID NO:28), 11 contiguous amino acid residues at or near the N-terminus of the wild-type NiV-G protein (SEQ ID NO:28), 12 contiguous amino acid residues at or near the N-terminus of the wild-type NiV-G protein (SEQ ID NO:28), 13 contiguous amino acid residues at or near the N-terminus of the wild-type NiV-G protein (SEQ ID NO:28), 14 contiguous amino acid residues at or near the N-terminus of the wild-type NiV-G protein (SEQ ID NO:28), up to 15 contiguous amino acid residues at or near the N-terminus of the wild-type NiV-G protein (SEQ ID NO:28), 16 contiguous amino acid residues at or near the N-terminus of the wild-type NiV-G protein (SEQ ID NO:28), 17 contiguous amino acid residues at or near the N-terminus of the wild-type NiV-G protein (SEQ ID NO:28), 18 contiguous amino acid residues at or near the N-terminus of the wild-type NiV-G protein (SEQ ID NO:28), 19 contiguous amino acid residues at or near the N-terminus of the wild-type NiV-G protein (SEQ ID NO:28), up to 20 contiguous amino acid residues at or near the N-terminus of the wild-type NiV-G protein (SEQ ID NO:28), 21 contiguous amino acid residues at or near the N-terminus of the wild-type NiV-G protein (SEQ ID NO:28), 22 contiguous amino acid residues at or near the N-terminus of the wild-type NiV-G protein (SEQ ID NO:28), 23 contiguous amino acid residues at or near the N-terminus of the wild-type NiV-G protein (SEQ ID NO:28), 24 contiguous amino acid residues at or near the N-terminus of the wild-type NiV-G protein (SEQ ID NO:28), up to 25 contiguous amino acid residues at or near the N-terminus of the wild-type NiV-G protein (SEQ ID NO:28), 26 contiguous amino acid residues at or near the N-terminus of the wild-type NiV-G protein (SEQ ID NO:28), 27 contiguous amino acid residues at or near the N-terminus of the wild-type NiV-G protein (SEQ ID NO:28), 28 contiguous amino acid residues at or near the N-terminus of the wild-type NiV-G protein (SEQ ID NO:28), 29 contiguous amino acid residues at or near the N-terminus of the wild-type NiV-G protein (SEQ ID NO:28), up to 30 contiguous amino acid residues at or near the N-terminus of the wild-type NiV-G protein (EQ ID NO:28), up to 31 contiguous amino acid residues at or near the N-terminus of the wild-type NiV-G protein (SEQ ID NO:28), 32 contiguous amino acid residues at or near the N-terminus of the wild-type NiV-G protein (SEQ ID NO:28), 33 contiguous amino acid residues at or near the N-terminus of the wild-type NiV-G protein (SEQ ID NO:28), 34 contiguous amino acid residues at or near the N-terminus of the wild-type NiV-G protein (SEQ ID NO:28), 35 contiguous amino acid residues at or near the N-terminus of the wild-type NiV-G protein (SEQ ID NO:28), up to 36 contiguous amino acid residues at or near the N-terminus of the wild-type NiV-G protein (EQ ID NO:28), up to 37 contiguous amino acid residues at or near the N-terminus of the wild-type NiV-G protein (EQ ID NO:28), up to 38 contiguous amino acid residues at or near the N-terminus of the wild-type NiV-G protein (EQ ID NO:28), up to 39 contiguous amino acid residues at or near the N-terminus of the wild-type NiV-G protein (EQ ID NO:28), or up to 40 contiguous amino acid residues at or near the N-terminus of the wild-type NiV-G protein (EQ ID NO:28).

In some embodiments, the mutant NiV-G protein has the amino acid sequence set forth in SEQ ID NO: 16 or 51 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:16 or 51. In particular embodiments, the G protein has the sequence of amino acids set forth in SEQ ID NO: 16 or 51.

In some embodiments, the targeted envelope protein contains a G protein or a functionally active variant or biologically active portion and an sdAb variable domain, in which the targeted envelope protein exhibits increased binding for another molecule that is different from the native binding partner of a wild-type G protein. In some embodiments, the molecule can be a protein expressed on the surface of desired target cell. In some embodiments, the increased binding to the other molecule is increased by greater than at or about 25%, at or about 30%, at or about 40%, at or about 50%, at or about 60%, at or about 70%, at or about 80%, at or about 90%, or at or about 100%. In particular embodiments, the binding confers re-targeted binding compared to the binding of a wild-type G protein in which a new or different binding activity is conferred.

2. Binding Domain

In some embodiments, the binding domain can be any agent that binds to a cell surface molecule on a target cells. In some embodiments, the binding domain can be an antibody or an antibody portion or fragment.

The binding domain may be modulated to have different binding strengths. For example, scFvs and antibodies with various binding strengths may be used to alter the fusion activity of the chimeric attachment proteins towards cells that display high or low amounts of the target antigen. For example DARPins with different affinities may be used to alter the fusion activity towards cells that display high or low amounts of the target antigen. Binding domains may also be modulated to target different regions on the target ligand, which will affect the fusion rate with cells displaying the target.

The binding domain may comprise a humanized antibody molecule, intact IgA, IgG, IgE or IgM antibody; bi- or multi-specific antibody (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. A targeting moiety can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs).

In some embodiments, the binding domain is a single chain molecule. In some embodiments, the binding domain is a single domain antibody. In some embodiments, the binding domain is a single chain variable fragment. In particular embodiments, the binding domain contains an antibody variable sequence (s) that is human or humanized.

In some embodiments, the binding domain is a single domain antibody. In some embodiments, the single domain antibody can be human or humanized In some embodiments, the single domain antibody or portion thereof is naturally occurring. In some embodiments, the single domain antibody or portion thereof is synthetic.

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In some embodiments, the heavy chain antibody devoid of light chains is referred to as VHH. In some embodiments, the single domain antibody antibodies have a molecular weight of 12-15 kDa. In some embodiments, the single domain antibody antibodies include camelid antibodies or shark antibodies. In some embodiments, the single domain antibody molecule is derived from antibodies raised in Camelidae species, for example in camel, llama, dromedary, alpaca, vicuna and guanaco. In some embodiments, the single domain antibody is referred to as immunoglobulin new antigen receptors (IgNARs) and is derived from cartilaginous fishes. In some embodiments, the single domain antibody is generated by splitting dimeric variable domains of human or mouse IgG into monomers and camelizing critical residues.

In some embodiments, the single domain antibody can be generated from phage display libraries. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, single domain antibodies a library of human single domain antibodies is synthetically generated by introducing diversity into one or more scaffolds.

In some embodiments, the C-terminus of the single domain antibody is attached to the C-terminus of the G protein or biologically active portion thereof. In some embodiments, the N-terminus of the single domain antibody is exposed on the exterior surface of the lipid bilayer. In some embodiments, the N-terminus of the single domain antibody binds to a cell surface molecule of a target cell. In some embodiments, the single domain antibody specifically binds to a cell surface molecule present on a target cell. In some embodiments, the cell surface molecule is a protein, glycan, lipid or low molecular weight molecule.

In some embodiments, the cell surface molecule of a target cell is an antigen or portion thereof. In some embodiments, the single domain antibody or portion thereof is an antibody having a single monomeric domain antigen binding/recognition domain that is able to bind selectively to a specific antigen. In some embodiments, the single domain antibody binds an antigen present on a target cell.

Exemplary cells include polymorphonuclear cells (also known as PMN, PML, PMNL, or granulocytes), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, immune effector cells, lymphocytes, macrophages, dendritic cells, natural killer cells, T cells, cytotoxic T lymphocytes, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes,

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g. hepatocyte), or a cadiac cell (e.g. cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g. a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoeietic stem cell (HSC), a liver cell or a fully differentiated cell. In some embodiments, the target cell is a CD3+ T cell, a CD4+ Tcell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell).

In some embodiments, the cell surface molecule is any one of CD8, CD4, asialoglycoprotein receptor 2 (ASGR2), transmembrane 4 L6 family member 5 (TM4SF5), low density lipoprotein receptor (LDLR) or asialoglycoprotein 1 (ASGR1).

In some embodiments, the G protein or functionally active variant or biologically active portion thereof is linked directly to the sdAb variable domain. In some embodiments, the targeted envelope protein is a fusion protein that has the following structure: (N′-single domain antibody-C′)-(C′-G protein-N′).

In some embodiments, the G protein or functionally active variant or biologically active portion thereof is linked indirectly via a linker to the the sdAb variable domain. In some embodiments, the linker is a peptide linker. In some embodiments, the linker is a chemical linker.

In some embodiments, the linker is a peptide linker and the targeted envelope protein is a fusion protein containing the G protein or functionally active variant or biologically active portion thereof linked via a peptide linker to the sdAb variable domain. In some embodiments, the targeted envelope protein is a fusion protein that has the following structure: (N′-single domain antibody-C′)-Linker-(C′-G protein-N′).

In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some embodiments, the peptide linker is a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

In particular embodiments, the linker is a flexible peptide linker. In some such embodiments, the linker is 1-20 amino acids, such as 1-20 amino acids predominantly composed of glycine. In some embodiments, the linker is 1-20 amino acids, such as 1-20 amino acids predominantly composed of glycine and serine. In some embodiments, the linker is a flexible peptide linker containing amino acids Glycine and Serine, referred to as GS-linkers. In some embodiments, the peptide linker includes the sequences GS, GGS, GGGGS (SEQ ID NO:43), GGGGGS (SEQ ID NO:41) or combinations thereof. In some embodiments, the polypeptide linker has the sequence (GGS)n, wherein n is 1 to 10. In some embodiments, the polypeptide linker has the sequence (GGGGS)n, (SEQ ID NO:42) wherein n is 1 to 10. In some embodiments, the polypeptide linker has the sequence (GGGGGS)n (SEQ ID NO:27), wherein n is 1 to 6.

3. Polynucleotides

Provided herein are polynucleotides comprising a nucleic acid sequence encoding a targeted envelope protein. In some embodiments, the polynucleotides comprise a nucleic acid sequence encoding a G protein or biologically active portion thereof. In some embodiments, the polynucleotides further comprise a nucleic acid sequence encoding a single domain antibody (sdAb) variable domain or biologically active portion thereof. The polynucleotides may include a sequence of nucleotides encoding any of the targeted envelope proteins described above. The polynucleotide can be a synthetic nucleic acid. Also provided are expression vector containing any of the provided polynucleotides.

In some of any embodiments, expression of natural or synthetic nucleic acids is typically achieved by operably linking a nucleic acid encoding the gene of interest to a promoter and incorporating the construct into an expression vector. In some embodiments, vectors can be suitable for replication and integration in eukaryotes. In some embodiments, cloning vectors contain transcription and translation terminators, initiation sequences, and promoters useful for expression of the desired nucleic acid sequence. In some of any embodiments, a plasmid comprises a promoter suitable for expression in a cell.

In some embodiments, the polynucleotides contain at least one promoter that is operatively linked to control expression of the targeted envelope protein containing the G protein and the single domain antibody (sdAb) variable domain. For expression of the targeted envelope protein, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

In some embodiments, additional promoter elements, e.g., enhancers, regulate the frequency of transcriptional initiation. In some embodiments, additional promoter elements are located in the region 30-110 bp upstream of the start site, although a number of promoters have recently been shown to contain functional elements downstream of the start site as well. In some embodiments, spacing between promoter elements frequently is flexible, so that promoter function is preserved when elements are inverted or moved relative to one another. In some embodiments, the thymidine kinase (tk) promoter, the spacing between promoter elements can be increased to 50 bp apart before activity begins to decline. In some embodiments, depending on the promoter, individual elements can function either cooperatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202 and 5,928,906).

In some embodiments, a suitable promoter is the immediate early cytomegalovirus (CMV) promoter sequence. In some embodiments, the promoter sequence is a strong constitutive promoter sequence capable of driving high levels of expression of any polynucleotide sequence operatively linked thereto. In some embodiments, a suitable promoter is Elongation Growth Factor-la (EF-1 a). In some embodiments, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

In some embodiments, the promoter is an inducible promoter. In some embodiments, the inducible promoter provides a molecular switch capable of turning on expression of the polynucleotide sequence which it is operatively linked when such expression is desired, or turning off the expression when expression is not desired. In some embodiments, inducible promoters comprise metallothionine promoter, a glucocorticoid promoter, a progesterone promoter, and a tetracycline promoter.

In some embodiments, exogenously controlled inducible promoters can be used to regulate expression of the G protein and single domain antibody (sdAb) variable domain. For example, radiation-inducible promoters, heat-inducible promoters, and/or drug-inducible promoters can be used to selectively drive transgene expression in, for example, targeted regions. In such embodiments, the location, duration, and level of transgene expression can be regulated by the administration of the exogenous source of induction.

In some embodiments, expression of the targeted envelope protein containing a G protein and single domain antibody (sdAb) variable domain is regulated using a drug-inducible promoter. For example, in some cases, the promoter, enhancer, or transactivator comprises a Lac operator sequence, a tetracycline operator sequence, a galactose operator sequence, a doxycycline operator sequence, a rapamycin operator sequence, a tamoxifen operator sequence, or a hormone-responsive operator sequence, or an analog thereof. In some instances, the inducible promoter comprises a tetracycline response element (TRE). In some embodiments, the inducible promoter comprises an estrogen response element (ERE), which can activate gene expression in the presence of tamoxifen. In some instances, a drug-inducible element, such as a TRE, can be combined with a selected promoter to enhance transcription in the presence of drug, such as doxycycline. In some embodiments, the drug-inducible promoter is a small molecule-inducible promoter.

Any of the provided polynucleotides can be modified to remove CpG motifs and/or to optimize codons for translation in a particular species, such as human, canine, feline, equine, ovine, bovine, etc. species. In some embodiments, the polynucleotides are optimized for human codon usage (i.e., human codon-optimized). In some embodiments, the polynucleotides are modified to remove CpG motifs. In other embodiments, the provided polynucleotides are modified to remove CpG motifs and are codon-optimized, such as human codon-optimized. Methods of codon optimization and CpG motif detection and modification are well-known. Typically, polynucleotide optimization enhances transgene expression, increases transgene stability and preserves the amino acid sequence of the encoded polypeptide.

In order to assess the expression of the targeted envelope protein, the expression vector to be introduced into a cell can also contain either a selectable marker gene or a reporter gene or both to facilitate identification and selection of expressing particles, e.g. viral particles. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000, FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of the desired polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

B. Fusogen (e.g. Henipavirus F Protein)

In some embodiments, the targeted lipid particle comprises one or more fusogens. In some embodiments, the targeted lipid particle contains an exogenous or overexpressed fusogen. In some embodiments, the fusogen is disposed in the lipid bilayer. In some embodiments, the fusogen facilitates the fusion of the targeted lipid particle to a membrane. In some embodiments, the membrane is a plasma cell membrane.

In some embodiments, fusogens comprise protein based, lipid based, and chemical based fusogens. In some embodiments, the targeted lipid particle comprises a first fusogen comprising a protein fusogen and a second fusogen comprising a lipid fusogen or chemical fusogen. In some embodiments, the fusogen binds fusogen binding partner on a target cell surface.

In some embodiments, the fusogen comprises a protein with a hydrophobic fusion peptide domain. In some embodiments, the fusogen comprises a henipavirus F protein molecule or biologically active portion thereof. In some embodiments, the Henipavirus F protein is a Hendra (Hey) virus F protein, a Nipah (NiV) virus F-protein, a Cedar (CedPV) virus F protein, a Mojiang virus F protein or a bat Paramyxovirus F protein or a biologically active portion thereof.

Table 4 provides non-limiting examples of F proteins. In some embodiments, the N-terminal hydrophobic fusion peptide domain of the F protein molecule or biologically active portion thereof is exposed on the outside of lipid bilayer.

F proteins of henipaviruses are encoded as F₀ precursors containing a signal peptide (e.g. corresponding to amino acid residues 1-26 of SEQ ID NO:1). Following cleavage of the signal peptide, the mature F₀ (e.g. SEQ ID NO:2) is transported to the cell surface, then endocytosed and cleaved by cathepsin L (e.g. between amino acids 109-110 of SEQ ID NO:1) into the mature fusogenic subunits F1 (e.g. corresponding to amino acids 110-546 of SEQ ID NO:1; set forth in SEQ ID NO:4) and F2 (e.g. corresponding to amino acid residues 27-109 of SEQ ID NO:1; set forth in SEQ ID NO:3). The F1 and F2 subunits are associated by a disulfide bond and recycled back to the cell surface. The F1 subunit contains the fusion peptide domain located at the N terminus of the F1 subunit (e.g. .g. corresponding to amino acids 110-129 of SEQ ID NO:1) where it is able to insert into a cell membrane to drive fusion. In particular cases, fusion activity is blocked by association of the F protein with G protein, until G engages with a target molecule resulting in its disassociation from F and exposure of the fusion peptide to mediate membrane fusion.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019. 93(13):e00577-19). In some aspects or the provided re-targeted lipid particles, the F protein is heterologous to the G protein, i.e. the F and G protein or biologically active portions are from different henipavirus species. For example, the F protein is from Hendra virus and the G protein is from Nipah virus. In other aspects, the F protein can be a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. 2019). In some cases, the chimeric F protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus. F protein sequences disclosed herein are predominantly disclosed as expressed sequences including an N-terminal signal sequence. As such N-terminal signal sequences are commonly cleaved co- or post-translationally, the mature protein sequences for all F protein sequences disclosed herein are also contemplated as lacking the N-terminal signal sequence.

TABLE 4
Henipavirus F sequence clusters. Column 1, Genbank ID includes the Genbank ID of
the whole genome sequence of the virus that is the centroid sequence of the cluster. Column 2,
Nucleotides of CDS provides the nucleotides corresponding to the CDS of the gene in the whole
genome. Column 3, Full Gene Name, provides the full name of the gene including Genbank ID,
virus species, strain, and protein name. Nipah virus F protein is >80% identical to that of
Hendra virus and is found within the same sequence cluster. Column 4, Sequence, provides the
amino acid sequence of the gene. Column 5, #Sequences/Cluster, provides the number of
sequences that cluster with this centroid sequence. Column 6 provides the SEQ ID numbers for
the described sequences.

| | | | | | | SEQ |
|---|---|---|---|---|---|---|
| | | | | | | ID |
| Gen- | Nucleotides | | | | SEQ | (without |
| bank | of | Full Gene | | #Sequences/ | ID | signal |
| ID | CDS | Name | Sequence | Cluster | NO | sequence) |
| AF | 6618 | gb: AF017149\| | MATQEVRLKCLLCGIIVLVLSLEGLGILHYEK | 29 | 17 | 59 |
| 017 | - | Organism: Hen | LSKIGLVKGITRKYKIKSNPLTKDIVIKMIPNVS | | | |
| 149 | 8258 | dra virus\|Strain | NVSKCTGTVMENYKSRLTGILSPIKGAIELYN | | | |
| | | Name: UNKN | NNTHDLVGDVKLAGVVMAGIAIGIATAAQIT | | | |
| | | OWN- | AGVALYEAMKNADNINKLKSSIESTNEAVVK | | | |
| | | AF017149\|Prot | LQETAEKTVYVLTALQDYINTNLVPTIDQISC | | | |
| | | ein | KQTELALDLALSKYLSDLLFVFGPNLQDPVSN | | | |
| | | Name: fusion\|G | SMTIQAISQAFGGNYETLLRTLGYATEDFDDL | | | |
| | | ene Symbol: F | LESDSIAGQIVYVDLSSYYIIVRVYFPILTEIQQ | | | |
| | | | AYVQELLPVSENNDNSEWISIVPNEVLIRNTLI | | | |
| | | | SNIEVKYCLITKKSVICNQDYATPMTASVREC | | | |
| | | | LTGSTDKCPRELVVSSHVPRFALSGGVLFANC | | | |
| | | | ISVTCQCQTTGRAISQSGEQTLLMIDNTTCTTV | | | |
| | | | VLGNIIISLGKYLGSINYNSESIAVGPPVYTDK | | | |
| | | | VDISSQISSMNQSLQQSKDYIKEAQKILDTVNP | | | |
| | | | SLISMLSMIILYVLSIAALCIGLITFISFVIVEKK | | | |
| | | | RGNYSRLDDRQVRPVSNGDLYYIGT | | | |
| Q9I | | Additional in | MVVILDKRCYCNLLILILMISECSVGILHYEKL | 1 | 2 | |
| H6 | | cluster: | SKIGLVKGVTRKYKIKSNPLTKDIVIKMIPNVS | | | |
| 3 | | sp\|Q9IH63\|FU | NMSQCTGSVMENYKTRLNGILTPIKGALEIYK | | | |
| | | S_NIPAV | NNTHDLVGDVRLAGVIMAGVAIGIATAAQIT | | | |
| | | Fusion | AGVALYEAMKNADNINKLKSSIESTNEAVVK | | | |
| | | glycoprotein | LQETAEKTVYVLTALQDYINTNLVPTIDKISC | | | |
| | | F0 OS = Nipah | KQTELSLDLALSKYLSDLLFVFGPNLQDPVSN | | | |
| | | virus | SMTIQAISQAFGGNYETLLRTLGYATEDFDDL | | | |
| | | | LESDSITGQIIYVDLSSYYIIVRVYFPILTEIQQA | | | |
| | | | YIQELLPVSFNNDNSEWISIVPNFILVRNTLISN | | | |
| | | | IEIGFCLITKRSVICNQDYATPMTNNMRECLTG | | | |
| | | | STEKCPRELVVSSHVPRFALSNGVLFANCISVT | | | |
| | | | CQCQTTGRAISQSGEQTLLMIDNTTCPTAVLG | | | |
| | | | NVIISLGKYLGSVNYNSEGIAIGPPVFTDKVDI | | | |
| | | | SSQISSMNQSLQQSKDYIKEAQRLLDTVNPSLI | | | |
| | | | SMLSMIILYVLSIASLCIGLITFISFIIVEKKRNT | | | |
| | | | YSRLEDRRVRPTSSGDLYYIGT | | | |
| JQ | 6129 | gb: JQ001776: 6 | MSNKRTTVLIIISYTLFYLNNAAIVGFDFDKLN | 3 | 24 | 57 |
| 001 | - | 129- | KIGVVQGRVLNYKIKGDPMTKDLVLKFIPNIV | | | |
| 776 | 8166 | 8166\|Organism: | NITECVREPLSRYNETVRRLLLPIHNMLGLYL | | | |
| | | Cedar | NNTNAKMTGLMIAGVIMGGIAIGIATAAQITA | | | |
| | | virus\|Strain | GFALYEAKKNTENIQKLTDSIMKTQDSIDKLT | | | |
| | | Name: CG1a\|Pr | DSVGTSILILNKLQTYINNQLVPNLELLSCRQN | | | |
| | | otein | KOEFDLMLTKYLVDLMTVIGPNINNPVNKDM | | | |
| | | Name: fusion | TIQSLSLLFDGNYDIMMSELGYTPQDFLDLIES | | | |
| | | glycoprotein\|G | KSITGQIIYVDMENLYVVIRTYLPTHEVPDAQI | | | |
| | | ene Symbol: F | YEFNKITMSSNGGEYLSTIPNFILIRGNYMSNI | | | |
| | | | DVATCYMTKASVICNQDYSLPMSQNLRSCYQ | | | |
| | | | GETEYCPVEAVIASHSPRFALTNGVIFANCINT | | | |
| | | | ICRCQDNGKTITQNINQFVSMIDNSTCNDVMV | | | |
| | | | DKFTIKVGKYMGRKDINNINIQIGPQIIIDKVD | | | |
| | | | LSNEINKMNQSLKDSIFYLREAKRILDSVNISLI | | | |
| | | | SPSVQLFLIIISVLSFIILLIIIVYLYCKSKHSYKY | | | |
| | | | NKFIDDPDYYNDYKRERINGKASKSNNIYYV | | | |
| | | | GD | | | |
| NC_ | 5950 | gb: NC_025352: | MALNKNMFSSLFLGYLLVYATTVQSSIHYDS | 2 | 25 | 60 |
| 02 | - | 5950- | LSKVGVIKGLTYNYKIKGSPSTKLMVVKLIPNI | | | |
| 535 | 8712 | 8712\|Organism: | DSVKNCTQKQYDEYKNLVRKALEPVKMAID | | | |
| 2 | | Mojiang | TMLNNVKSGNNKYRFAGAIMAGVALGVATA | | | |
| | | virus\|Strain | ATVTAGIALHRSNENAQAIANMKSAIQNTNE | | | |
| | | Name: Tonggua | AVKQLQLANKQTLAVIDTIRGEINNNIIPVINQ | | | |
| | | n1\|Protein | LSCDTIGLSVGIRLTQYYSEIITAFGPALQNPV | | | |
| | | Name: fusion | NTRITIQAISSVFNGNFDELLKIMGYTSGDLYE | | | |
| | | protein\|Gene | ILHSELIRGNIIDVDVDAGYIALEIEFPNLTLVP | | | |
| | | Symbol: F | NAVVQELMPISYNIDGDEWVTLVPRFVLTRTT | | | |
| | | | LLSNIDTSRCTITDSSVICDNDYALPMSHELIG | | | |
| | | | CLQGDTSKCAREKVVSSYVPKFALSDGLVYA | | | |
| | | | NCLNTICRCMDTDTPISQSLGATVSLLDNKRC | | | |
| | | | SVYQVGDVLISVGSYLGDGEYNADNVELGPPI | | | |
| | | | VIDKIDIGNQLAGINQTLQEAEDYIEKSEEFLK | | | |
| | | | GVNPSIITLGSMVVLYIFMILIAIVSVIALVLSIK | | | |
| | | | LTVKGNVVRQQFTYTQHVPSMENINYVSH | | | |
| NC_ | 6865 | gb: NC_025256: | MKKKTDNPTISKRGHNHSRGIKSRALLRETDN | 2 | 26 | 58 |
| 02 | - | 6865- | YSNGLIVENLVRNCHHPSKNNLNYTKTQKRD | | | |
| 525 | 8853 | 8853\|Organism: | STIPYRVEERKGHYPKIKHLIDKSYKHIKRGKR | | | |
| 6 | | Bat | RNGHNGNIITIILLLILILKTQMSEGAIHYETLS | | | |
| | | Paramyxovirus | KIGLIKGITREYKVKGTPSSKDIVIKLIPNVTGL | | | |
| | | Eid_he1/GH- | NKCTNISMENYKEQLDKILIPIINNIIELYANSTK | | | |
| | | M74a/GHA/20 | SAPGNARFAGVIIAGVALGVAAAAQITAGIAL | | | |
| | | 09\|Strain | HEARQNAERINLLKDSISATNNAVAELQEATG | | | |
| | | Name: BatPV/E | GIVNVITGMQDYINTNLVPQIDKLQCSQIKTA | | | |
| | | id_he1/GH- | LDISLSQYYSEILTVFGPNLQNPVTTSMSIQAIS | | | |
| | | M74a/GHA/20 | QSFGGNIDLLLNLLGYTANDLLDLLESKSITG | | | |
| | | 09\|Protein | QITYINLEHYFMVIRVYYPIMTTISNAYVQELI | | | |
| | | Name: fusion | KISFNVDGSEWVSLVPSYILIRNSYLSNIDISEC | | | |
| | | protein\|Gene | LITKNSVICRHDFAMPMSYTLKECLTGDTEKC | | | |
| | | Symbol: F | PREAVVTSYVPRFAISGGVIYANCLSTTCQCY | | | |
| | | | QTGKVIAQDGSQTLMMIDNQTCSIVRIEEILIS | | | |
| | | | TGKYLGSQEYNTMHVSVGNPVFTDKLDITSQI | | | |
| | | | SNINQSIEQSKFYLDKSKAILDKINLNLIGSVPI | | | |
| | | | SILFIIAILSLILSIITFVIVMIIVRRYNKYTPLINS | | | |
| | | | DPSSRRSTIQDVYIIPNPGEHSIRSAARSIDRDR | | | |
| | | | D | | | |

In some embodiments, the F protein is encoded by a nucleotide sequence that encodes the sequence set forth by any one of SEQ ID NOs: 1, 2, 17, 24, 25, 26 or 57-60 or is a functionally active variant or a biologically active portion thereof that has a sequence that is at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 1, 2, 17, 24, 25, 26 or 57-60. In particular embodiments, the F protein or the functionally active variant or biologically active portion thereof retains fusogenic activity in conjunction with a Henipavirus G protein, such as a G protein set forth in Section I.A (e.g. NiV-G or HeV-G). Fusogenic activity includes the activity of the F protein in conjunction with a Henipavirus G protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F). In particular embodiments, the F protein of the functionally active variant or biologically active portion retains the cleavage site cleaved by cathepsin L (e.g. corresponding to the cleavage site between amino acids 109-110 of SEQ ID NO:1).

In particular embodiments, the F protein has the sequence of amino acids set forth in SEQ ID NO: 1, SEQ ID NO:2, SEQ ID NO:17, SEQ ID NO: 24, SEQ ID NO:25, SEQ ID NO: 26, SEQ ID NO: 57, SEQ ID NO: 57, SEQ ID NO: 58, SEQ ID NO: 59, or SEQ ID NO: 60 or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 1, SEQ ID NO:2, SEQ ID NO:17, SEQ ID NO: 24, SEQ ID NO:25, SEQ ID NO: 26, SEQ ID NO: 57, SEQ ID NO: 57, SEQ ID NO: 58, SEQ ID NO: 59, or SEQ ID NO: 60 and retains fusogenic activity in conjunction with a Henipavirus G protein (e.g., NiV-G or HeV-G). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 1, SEQ ID NO:2, SEQ ID NO:17, SEQ ID NO: 24, SEQ ID NO:25, SEQ ID NO: 26, SEQ ID NO: 57, SEQ ID NO: 57, SEQ ID NO: 58, SEQ ID NO: 59, or SEQ ID NO: 60 and retains fusogenic activity in conjunction with a Henipavirus G protein (e.g., NiV-G or HeV-G).

Reference to retaining fusogenic activity includes activity (in conjunction with a Henipavirus G protein) that between at or about 10% and at or about 150% or more of the level or degree of binding of the corresponding wild-type F protein, such as set forth in SEQ ID NO: 1, SEQ ID NO:2, SEQ ID NO:17, SEQ ID NO: 24, SEQ ID NO:25, SEQ ID NO: 26, SEQ ID NO: 57, SEQ ID NO: 57, SEQ ID NO: 58, SEQ ID NO: 59, or SEQ ID NO: 60, such as at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type f protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the F protein is a mutant F protein that is a functionally active fragment or a biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (Hey) virus F protein, a Nipah (NiV) virus F-protein, a Cedar (CedPV) virus F protein, a Mojiang virus F protein or a bat Paramyxovirus F protein. In some embodiments, the wild-type F protein is encoded by a sequence of nucleotides that encodes any one of SEQ ID NO: 1, 2, 17, 24, 25, 26, or 57-60.

In some embodiments, the mutant F protein is a biologically active portion of a wild-type F protein that is an N-terminally and/or C-terminally truncated fragment. In some embodiments, the mutant F protein or the biologically active portion of a wild-type F protein thereof comprises one or more amino acid substitutions. In some embodiments, the mutations described herein can improve transduction efficiency. In some embodiments, the mutations described herein can increase fusogenic capacity. Exemplary mutations include any as described, see e.g. Khetawat and Broder 2010 Virology Journal 7:312; Witting et al. 2013 Gene Therapy 20:997-1005; published international; patent application No. WO/2013/148327.

In some embodiments, the mutant F protein is a biologically active portion that is truncated and lacks up to 20 contiguous amino acid residues at or near the C-terminus of the wild-type F protein, such as a wild-type F protein encoded by a sequence of nucleotides encoding the F protein set forth in any one of SEQ ID NOS: 1, 17, 24, 25 or 26. In some embodiments, the mutant F protein is truncated and lacks up to 19 contiguous amino acids, such as up to 18, 17, 16, 15, 14, 13, 12, 11, 10, 9, 8, 7, 6, 5, 4, 3, 2, or 1 contiguous amino acids at the C-terminus of the wild-type F protein.

In some embodiments, the F protein or the functionally active variant or biologically active portion thereof comprises an F1 subunit or a fusogenic portion thereof. In some embodiments, the F1 subunit is a proteolytically cleaved portion of the F0 precursor. In some embodiments, the F0 precursor is inactive. In some embodiments, the cleavage of the F0 precursor forms a disulfide-linked F1+F2 heterodimer. In some embodiments, the cleavage exposes the fusion peptide and produces a mature F protein. In some embodiments, the cleavage occurs at or around a single basic residue. In some embodiments, the cleavage occurs at Arginine 109 of NiV-F protein. In some embodiments, cleavage occurs at Lysine 109 of the Hendra virus F protein.

In some embodiments, the F protein is a wild-type Nipah virus F (NiV-F) protein or is a functionally active variant or biologically active portion thereof. In some embodiments, the F₀ precursor is encoded by a sequence of nucleotides encoding the sequence set forth in SEQ ID NO: 1. The encoding nucleic acid can encode a signal peptide sequence that has the sequence MVVILDKRCY CNLLILILMI SECSVG (SEQ ID NO: 34). In some embodiments, the F protein has the sequence set forth in SEQ ID NO:2. In some examples, the F protein is cleaved into an F1 subunit comprising the sequence set forth in SEQ ID NO:4 and an F2 subunit comprising the sequence set forth in SEQ ID NO: 3.

In some embodiments, the F protein is a NiV-F protein that is encoded by a sequence of nucleotides encoding the sequence set forth in SEQ ID NO:1, or is a functionally active variant or biologically active portion thereof that has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 1. In some embodiments, the NiV-F-protein has the sequence of set forth in SEQ ID NO: 2, or is a functionally active variant or a biologically active portion thereof that has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 2. In particular embodiments, the F protein or the functionally active variant or biologically active portion thereof retains the cleavage site cleaved by cathepsin L (e.g. corresponding to the cleavage site between amino acids 109-110 of SEQ ID NO:1).

In some embodiments, the F protein or the functionally active variant or the biologically active portion thereof includes an F1 subunit that has the sequence set forth in SEQ ID NO: 4, or an amino acid sequence having, at least at or about 80%, at least at or about 81%, at least at or about 82%, at least at or about 83%, at least at or about 84%, at least at or about 85%, at or about 86%, at least at or about 87%, at least at or about 88%, or at least at or about 89% at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:4.

In some embodiments, the F protein or the functionally active variant or biologically active portion thereof includes an F2 subunit that has the sequence set forth in SEQ ID NO: 3, or an amino acid sequence having, at least at or about 80%, at least at or about 81%, at least at or about 82%, at least at or about 83%, at least at or about 84%, at least at or about 85%, at or about 86%, at least at or about 87%, at least at or about 88%, or at least at or about 89% at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:3.

In some embodiments, the F protein is a mutant NiV-F protein that is a biologically active portion thereof that is truncated and lacks up to 20 contiguous amino acid residues at or near the C-terminus of the wild-type NiV-F protein (e.g. set forth SEQ ID NO:2). In some embodiments, the mutant NiV-F protein comprises an amino acid sequence set forth in SEQ ID NO:5. In some embodiments, the mutant NiV-F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 5. In some embodiments, the mutant F protein contains an F1 protein that has the sequence set forth in SEQ ID NO:6. In some embodiments, the mutant F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 6.

In some embodiments, the F protein is a mutant NiV-F protein that is a biologically active portion thereof that comprises a 20 amino acid truncation at or near the C-terminus of the wild-type NiV-F protein (SEQ ID NO:2); and a point mutation on an N-linked glycosylation site. In some embodiments, the mutant NiV-F protein comprises an amino acid sequence set forth in SEQ ID NO: 7. In some embodiments, the mutant NiV-F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 7.

In some embodiments, the F protein is a mutant NiV-F protein that is a biologically active portion thereof that comprises a 22 amino acid truncation at or near the C-terminus of the wild-type NiV-F protein (SEQ ID NO:2). In some embodiments, the NiV-F protein is encoded by a nucleotide sequence that encodes the sequence set forth in SEQ ID NO: 8. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 8. In particular embodiments, the variant F protein is a mutant Niv-F protein that has the sequence of amino acids set forth in SEQ ID NO:23. In some embodiments, the NiV-F proteins is encoded by a a sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 23.

C. Lipid Bilayer

In some embodiments, the targeted lipid particle includes a naturally derived bilayer of amphipathic lipids that encloses lumen or cavity. In some embodiments, the targeted lipid particle comprises a lipid bilayer as the outermost surface. In some embodiments, the lipid bilayer encloses a lumen. In some embodiments, the lumen is aqueous. In some embodiments, the lumen is in contact with the hydrophilic head groups on the interior of the lipid bilayer. In some embodiments, the lumen is a cytosol. In some embodiments, the cytosol contains cellular components present in a source cell. In some embodiments, the cytosol does not contain components present in a source cell. In some embodiments, the lumen is a cavity. In some embodiments, the cavity contains an aqueous environment. In some embodiments, the cavity does not contain an aqueous environment.

In some aspects, the lipid bilayer is derived from a source cell during a process to produce a lipid-containing particle. Exemplary methods for producing lipid-containing particles are provided in Section I.E. In some embodiments, the lipid bilayer includes membrane components of the cell from which the lipid bilayer is produced, e.g., phospholipids, membrane proteins, etc. In some embodiments, the lipid bilayer includes a cytosol that includes components found in the cell from which the micro-vesicle is produced, e.g., solutes, proteins, nucleic acids, etc., but not all of the components of a cell, e.g., they lack a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In some embodiments, the lipid bilayer is a viral envelope. In some embodiments, the viral envelope is obtained from a source cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins.

In other aspects, the lipid bilayer includes synthetic lipid complex. In some embodiments, the synthetic lipid complex is a liposome. In some embodiments, the lipid bilayer is a vesicular structure characterized by a phospholipid bilayer membrane and an inner aqueous medium. In some embodiments, the lipid bilayer has multiple lipid layers separated by aqueous medium. In some embodiments, the lipid bilayer forms spontaneously when phospholipids are suspended in an excess of aqueous solution. In some examples, the lipid components undergo self-rearrangement before the formation of closed structures and entrap water and dissolved solutes between the lipid bilayers.

In some embodiments, a targeted envelope protein and fusogen, such as any described above including any that are exogenous or overexpressed relative to the source cell, is disposed in the lipid bilayer.

In some embodiments, the targeted lipid particle comprises several different types of lipids. In some embodiments, the lipids are amphipathic lipids. In some embodiments, the amphipathic lipids are phospholipids. In some embodiments, the phospholipids comprise phosphatidylcholine, phosphatidylethanolamine, phosphatidylinositol, and phosphatidylserine. In some embodiments, the lipids comprise phospholipids such as phosphocholines and phosphoinositols. In some embodiments, the lipids comprise DMPC, DOPC, and DSPC.

In some embodiments, the bilayer may be comprised of one or more lipids of the same or different type. In some embodiments, the source cell comprises a cell selected from CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRCS cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

D. Exogenous Agent

In embodiments, the targeted lipid particle, such as a lentiviral vector, further comprises an agent that is exogenous relative to the source cell (hereinafter also called “cargo” or “payload”). In some embodiments, the exogenous agent is a protein or a nucleic acid (e.g., a DNA, a chromosome (e.g. a human artificial chromosome), an RNA, e.g., an mRNA or miRNA). In some embodiments, the exogenous agent is a nucleic acid that encodes a protein. The protein can be any protein as is desired for targeted delivery to a target cell. In some embodiments, the protein is a therapeutic agent or a diagnostic agent. In some embodiments, the protein is an antigen receptor for targeting cells expressed by or associated with a disease or condition, for instance a chimeric antigen receptor (CAR) or a T cell receptor (TCR). Reference to the coding sequence of a nucleic acid encoding the protein also is referred to herein as a payload gene. In some embodiments, the exogenous agent or the nucleic acid encoding the exogenous agent are present in the lumen of the non-cell particle.

In some embodiments, the exogenous agent or cargo comprises or encodes a cytosolic protein. In some embodiments the exogenous agent or cargo comprises or encodes a membrane protein. In some embodiments, the exogenous agent or cargo comprises or encodes a therapeutic agent. In some embodiments, the therapeutic agent is chosen from one or more of a protein, e.g., an enzyme, a transmembrane protein, a receptor, an antibody; a nucleic acid, e.g., DNA, a chromosome (e.g. a human artificial chromosome), RNA, mRNA, siRNA, miRNA, or a small molecule.

In embodiments, the exogenous agent is present at least, or no more than, 10, 20, 50, 100, 200, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In embodiments, the targeted lipid particle has an altered, e.g., increased or decreased level of one or more endogenous molecule, e.g., protein or nucleic acid (e.g., in some embodiments, endogenous relative to the source cell, and in some embodiments, endogenous relative to the target cell), e.g., due to treatment of the source cell, e.g., mammalian source cell with a siRNA or gene editing enzyme. In embodiments, the endogenous molecule is present at least, or no more than, 10, 20, 50, 100, 200, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In embodiments, the endogenous molecule (e.g., an RNA or protein) is present at a concentration of at least 1, 2, 3, 4, 5, 10, 20, 50, 100, 500, 10³, 5.0×10³, 10⁴, 5.0×10⁴, 10⁵, 5.0×10⁵, 10⁶, 5.0×10⁶, 1.0×10⁷, 5.0×10⁷, or 1.0×10⁸, greater than its concentration in the source cell. In embodiments, the endogenous molecule (e.g., an RNA or protein) is present at a concentration of at least 1, 2, 3, 4, 5, 10, 20, 50, 100, 500, 10³, 5.0×10³, 10⁴, 5.0×10⁴, 10⁵, 5.0×10⁵, 10⁶, 5.0×10⁶, 1.0×10⁷, 5.0×10⁷, or 1.0×10⁸ less than its concentration in the source cell.

In some embodiments, the targeted lipid particle delivers to a target cell at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the fusosome. In some embodiments, the targeted lipid particle that fuses with the target cell(s) delivers to the target cell an average of at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the lipid particles that fuse with the target cell(s). In some embodiments, the targeted lipid particle composition delivers to a target tissue at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the targeted lipid particle compositions.

In some embodiments, the exogenous agent or cargo is not expressed naturally in the cell from which the targeted lipid particle is derived. In some embodiments, the exogenous agent or cargo is expressed naturally in the cell from which the targeted lipid particle is derived. In some embodiments, the exogenous agent or cargo is loaded into the targeted lipid particle via expression in the cell from which the lipid particle is derived (e.g. expression from DNA or mRNA introduced via transfection, transduction, or electroporation). In some embodiments, the exogenous agent or cargo is expressed from DNA integrated into the genome or maintained episosomally. In some embodiments, expression of the exogenous agent or cargo is constitutive. In some embodiments, expression of the exogenous agent or cargo is induced. In some embodiments, expression of the exogenous agent or cargo is induced immediately prior to generating the targeted lipid particle. In some embodiments, expression of the exogenous agent or cargo is induced at the same time as expression of the fusogen.

In some embodiments, the exogenous agent or cargo is loaded into the lipid particle via electroporation into the lipid particle itself or into the cell from which the fusosome is derived. In some embodiments, the exogenous agent or cargo is loaded into the lipid particle via transfection (e.g., of a DNA or mRNA encoding the cargo) into the lipid particle itself or into the cell from which the lipid particle is derived.

In some embodiments, the exogenous agent or cargo may include one or more nucleic acid sequences, one or more polypeptides, a combination of nucleic acid sequences and/or polypeptides, one or more organelles, and any combination thereof. In some embodiments, the exogenous agent or cargo may include one or more cellular components. In some embodiments, the exogenous agent or cargo includes one or more cytosolic and/or nuclear components.

In some embodiments, the exogenous agent or cargo includes a nucleic acid, e.g., DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), IncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprogramming RNAs, aptamers, and any combination thereof. In some embodiments, the nucleic acid is a wild-type nucleic acid. In some embodiments, the protein is a mutant nucleic acid. In some embodiments the nucleic acid is a fusion or chimera of multiple nucleic acid sequences.

In some embodiments, the exogenous agent or cargo may include a nucleic acid. For example, the exogenous agent or cargo may comprise RNA to enhance expression of an endogenous protein, or a siRNA or miRNA that inhibits protein expression of an endogenous protein. For example, the endogenous protein may modulate structure or function in the target cells. In some embodiments, the cargo may include a nucleic acid encoding an engineered protein that modulates structure or function in the target cells. In some embodiments, the exogenous agent or cargo is a nucleic acid that targets a transcriptional activator that modulate structure or function in the target cells.

In some embodiments, the exogenous agent or cargo is or encodes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, and any combination thereof. In some embodiments the protein targets a protein in the cell for degradation. In some embodiments the protein targets a protein in the cell for degradation by localizing the protein to the proteasome. In some embodiments, the protein is a wild-type protein. In some embodiments, the protein is a mutant protein. In some embodiments the protein is a fusion or chimeric protein.

In some embodiments, the exogenous agent or cargo is a small molecule, e.g., ions (e.g. Ca²⁺, Cl-, Fe²⁺), carbohydrates, lipids, reactive oxygen species, reactive nitrogen species, isoprenoids, signaling molecules, heme, polypeptide cofactors, electron accepting compounds, electron donating compounds, metabolites, ligands, and any combination thereof. In some embodiments the small molecule is a pharmaceutical that interacts with a target in the cell. In some embodiments the small molecule targets a protein in the cell for degradation. In some embodiments the small molecule targets a protein in the cell for degradation by localizing the protein to the proteasome. In some embodiments that small molecule is a proteolysis targeting chimera molecule (PROTAC).

In some embodiments, the exogenous agent or cargo includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g. Cas9-gRNA complex); multiple transcription factors, multiple epigenetic factors, reprogramming factors (e.g. Oct4, Sox2, cMyc, and Klf4); multiple regulatory RNAs; and any combination thereof.

In some embodiments, the exogenous agent or cargo includes one or more organelles, e.g., chondrisomes, mitochondria, lysosomes, nucleus, cell membrane, cytoplasm, endoplasmic reticulum, ribosomes, vacuoles, endosomes, spliceosomes, polymerases, capsids, acrosome, autophagosome, centriole, glycosome, glyoxysome, hydrogenosome, melanosome, mitosome, myofibril, cnidocyst, peroxisome, proteasome, vesicle, stress granule, networks of organelles, and any combination thereof.

In some embodiments, the exogenous agent is or encodes a cytosolic protein, e.g., a protein that is produced in the recipient cell and localizes to the recipient cell cytoplasm. In some embodiments, the exogenous agent is or encodes a secreted protein, e.g., a protein that is produced and secreted by the recipient cell. In some embodiments, the exogenous agent is or encodes a nuclear protein, e.g., a protein that is produced in the recipient cell and is imported to the nucleus of the recipient cell. In some embodiments, the exogenous agent is or encodes an organellar protein (e.g., a mitochondrial protein), e.g., a protein that is produced in the recipient cell and is imported into an organelle (e.g., a mitochondrial) of the recipient cell. In some embodiments, the protein is a wild-type protein or a mutant protein. In some embodiments the protein is a fusion or chimeric protein.

In some embodiments, the exogenous agent is capable of being delivered to a hepatocyte or liver cell. In some embodiments, the exogenous agents or cargo can be delivered to treat a disease or disorder in a hepatocyte or liver cell.

In some embodiments, the exogenous agent is encoded by a gene from among OTC, CPS1, NAGS, BCKDHA, BCKDHB, DBT, DLD, MUT, MMAA, MMAB, MMACHC, MMADHC, MCEE, PCCA, PCCB, UGT1A1, ASS1, PAH, PAL, ATP8B1, ABCB11, ABCB4, TJP2, IVD, GCDH, ETFA, ETFB, ETFDH, ASL, D2HGDH, HMGCL, MCCC1, MCCC2, ABCD4, HCFC1, LNBRD1, ARG1, SLC25A15, SLC25A13, ALAD, CPDX, HMBS, PPDX, BTD, HLCS, PC, SLC7A7, CPT2, ACADM, ACADS, ACADVL, AGL, G6PC, GBE1, PHKA1, PHKA2, PHKB, PHKG2, SLC37A4, PMM2, CBS, FAH, TAT, GALT, GALK1, GALE, G6PD, SLC3A1, SLC7A9, MTHFR, MTR, MTRR, ATP7B, HPRT1, HJV, HAMP, JAG1, TTR, AGXT, LIPA, SERPING1, HSD17B4, UROD, HFE, LPL, GRHPR, HOGA1, LDLR, ACAD8, ACADSB, ACAT1, ACSF3, ASPA, AUH, DNAJC19, ETHE1, FBP1, FTCD, GSS, HIBCH, IDH2, L2HGDH, MLYCD, OPA3, OPLAH, OXCT1, POLG, PPM1K, SERAC1, SLC25A1, SUCLA2, SUCLG1, TAZ, AGK, CLPB, TMEM70, ALDH18A1, OAT, CASA, GLUD1, GLUL, UMPS, SLC22A5, CPT1A, HADHA, HADH, SLC52A1, SLC52A2, SLC52A3, HADHB, GYS2, PYGL, SLC2A2, ALG1, ALG2, ALG3, ALG6, ALG8, ALG9, ALG11, ALG12, ALG13, ATP6V0A2, B3GLCT, CHST14, COG1, COG2, COG4, COG5, COG6, COG7, COG8, DOLK, DHDDS, DPAGT1, DPM1, DPM2, DPM3, G6PC3, GFPT1, GMPPA, GMPPB, MAGT1, MAN1B1, MGAT2, MOGS, MPDU1, MPI, NGLY1, PGM1, PGM3, RFT1, SEC23B, SLC35A1, SLC35A2, SLC35C1, SSR4, SRD5A3, TMEM165, TRIP11, TUSC3, ALG14, B4GALT1, DDOST, NUS1, RPN2, SEC23A, SLC35A3, ST3GAL3, STT3A, STT3B, AGA, ARSA, ARSB, ASAH1, ATP13A2, CLN3, CLNS, CLN6, CLN8, CTNS, CTSA, CTSD, CTSF, CTSK, DNAJCS, FUCA1, GAA, GALC, GALNS, GLA, GLB1, GM2A, GNPTAB, GNPTG, GNS, GRN, GUSB, HEXA, HEXB, HGSNAT, HYAL1, IDS, IDUA, KCTD7, LAMP2, MAN2B1, MANBA, MCOLN1, MFSD8, NAGA, NAGLU, NEU1 NPC1, NPC2, SGSH, PPT1, PSAP, SLC17A5, SMPD1, SUMF1, TPP1, AHCY, GNMT, MAT1A, GCH1, PCBD1, PTS, QDPR, SPR, DNAJC12, ALDH4A1, PRODH, HPD, GBA, HGD, AMN, CD320, CUBN, GIF, TCN1, TCN2, PREPL, PHGDH, PSAT1, PSPH, AMT, GCSH, GLDC, LIAS, NFU1, SLC6A9, SLC2A1, ATP7A, AP1S1, CP, SLC33A1, PEX7 PHYH, AGPS, GNPAT, ABCD1, ACOX1, PEX1, PEX2, PEX3, PEXS, PEX6, PEX10, PEX12, PEX13, PEX14, PEX16, PEX19, PEX26, AMACR, ADA, ADSL, AMPD1, GPHN, MOCOS, MOCS1, PNP, XDH, SUOX, OGDH, SLC25A19, DHTKD1, SLC13A5, FH, DLAT, MPC1, PDHA1, PDHB, PDHX, PDP1, ABCC2, SLCO1B1, SLCO1B3, HFE2, ADAMTS13, PYGM, COL1A2, TNFRSF11B, TSC1, TSC2, DHCR7, PGK1, VLDLR, KYNU, F5, C3, COL4A1, CFH, SLC12A2, GK, SFTPC, CRTAP, P3H1, COL7A1, PKLR, TALDO1, TF, EPCAM, VHL, GC, SERPINA1, ABCC6, F8, F9, ApoB, PCSK9, LDLRAP1, ABCGS, ABCG8, LCAT, SPINKS, or GNE.

In some embodiments, the exogenous agent is encoded by a gene from among OTC, CPS1, NAGS, BCKDHA, BCKDHB, DBT, DLD, MUT, MMAA, MMAB, MMACHC, MMADHC, MCEE, PCCA, PCCB, UGT1A1, ASS1, PAL, PAH, ATP8B1, ABCB11, ABCB4, TJP2, IVD, GCDH, ETFA, ETFB, ETFDH, ASL, D2HGDH, HMGCL, MCCC1, MCCC2, ABCD4, HCFC1, LMBRD1, ARG1, SLC25A15, SLC25A13, ALAD, CPDX, HMBS, PPDX, BTD, HLCS, PC, SLC7A7, CPT2, ACADM, ACADS, ACADVL, AGL, G6PC, GBE1, PHKA1, PHKA2, PHKB, PHKG2, SLC37A4, PMM2, CBS, FAH, TAT, GALT, GALK1, GALE, G6PD, SLC3A1, SLC7A9, MTHFR, MTR, MTRR, ATP7B, HPRT1, HJV, HAMP, JAG1, TTR, AGXT, LIPA, SERPING1, HSD17B4, UROD, HFE, LPL, GRHPR, HOGA1, or LDLR. In some embodiments, the exogenous agent is the enzyme phenylalanine ammonia lyase (PAL).

In some embodiments, the exogenous agents or cargo can be delivered to treat and disease or indication listed in Table 5. In some embodiments, the indications are specific for a liver cell or hepatocyte.

In some embodiments, the exogenous agent comprises a protein of Table 5 below. In some embodiments, the exogenous agent comprises the wild-type human sequence of any of the proteins of Table 5, a functional fragment thereof (e.g., an enzymatically active fragment thereof), or a functional variant thereof. In some embodiments, the exogenous agent comprises an amino acid sequence having at least 70%, 75%, 80%, 85%, 90%, 95%, 96%, 97%, 98%, or 99%, identity to an amino acid sequence of Table 5, e.g., a Uniprot Protein Accession Number sequence of column 4 of Table 5 or an amino acid sequence of column 5 of Table 5. In some embodiments, the payload gene encoding an exogenous agent encodes an amino acid sequence having at least 70%, 75%, 80%, 85%, 90%, 95%, 96%, 97%, 98%, or 99%, identity to an amino acid sequence of Table 5. In some embodiments, the payload gene encoding an exogenous agent has a nucleic acid sequence having at least 70%, 75%, 80%, 85%, 90%, 95%, 96%, 97%, 98%, or 99%, identity to a nucleic acid sequence of Table 5, e.g., an Ensemble Gene Accession Number of column 3 of Table 5.

TABLE 5
The first column lists exogenous agents that can be delivered to treat the indications in the sixth column, according to the
methods and uses herein. Each Uniprot accession number of Table 5 is herein incorporated by reference in its entirety.

| | | Ensembl | | Amino Acid | | |
|---|---|---|---|---|---|---|
| | | Gene(s) | | Sequence | | |
| | | Accession | Uniprot | (first Uniprot | | |
| | Entrez | Number | Protein(s) | Accession | | |
| | Accession | (ENSG0000 + | Accession | Number) | | |
| Gene | Number | number shown) | Number | SEQ ID NO | Disease/Disorder | Category |
| OTC | 5009 | 0036473 | P00480 | 61 | ornithine | Urea cycle disorder |
| | | | | | transcarbamylase | |
| | | | | | (OTC) deficiency | |
| CPS1 | 1373 | 0021826 | P31327, | 62 | carbamoyl | Urea cycle disorder |
| | | | Q6PEK7, | | phosphate | |
| | | | B7ZAW0, | | synthetase I | |
| | | | A0A024R454 | | (CPSI) deficiency | |
| NAGS | 162417 | 0161653 | Q8N159, | 63 | N-acetylglutamate | Urea cycle disorder |
| | | | Q2NKP2 | | synthase (NAGS) | |
| | | | | | deficiency | |
| BCKDHA | 593 | 0248098 | A0A024R0K3, | 64 | maple syrup urine | Organic acidemia |
| | | | P12694, | | disease (MSUD); | |
| | | | Q59EI3 | | Classic Maple | |
| | | | | | Syrup Urine | |
| | | | | | Disease (CMSUD) | |
| BCKDHB | 594 | 0083123 | A0A140VKB3, | 65 | maple syrup urine | Organic acidemia |
| | | | P21953, | | disease (MSUD); | |
| | | | B4E2N3, | | Classic Maple | |
| | | | B7ZB80 | | Syrup Urine | |
| | | | | | Disease (CMSUD) | |
| DBT | 1629 | 0137992 | P11182 | 66 | maple syrup urine | Organic acidemia |
| | | | | | disease (MSUD); | |
| | | | | | Classic Maple | |
| | | | | | Syrup Urine | |
| | | | | | Disease (CMSUD) | |
| DLD | 1738 | 0091140 | A0A024R713, | 67 | maple syrup urine | Urea cycle disorder |
| | | | P09622, | | disease (MSUD) | |
| | | | E9PEX6 | | Dihydrolipoamide | |
| | | | | | dehydrogenase | |
| | | | | | deficiency | |
| MUT | 4594 | 0146085 | A0A024RD82, | 68 | methylmalonic | Organic acidemia |
| | | | B2R6K1, | | acidemia due to | |
| | | | P22033 | | methylmalonyl- | |
| | | | | | CoA mutase | |
| | | | | | deficiency | |
| MMAA | 166785 | 0151611 | Q8IVH4 | 69 | cobalamin A | Organic acidemia |
| | | | | | deficiency | |
| | | | | | (methylmalonic | |
| | | | | | acidemia) | |
| MMAB | 326625 | 0139428 | Q96EY8 | 70 | cobalamin B | Organic acidemia |
| | | | | | deficiency | |
| | | | | | (methylmalonic | |
| | | | | | acidemia) | |
| MMACHC | 25974 | 0132763 | A0A0C4DGU2, | 71 | cobalamin C | Organic acidemia |
| | | | Q9Y4U1 | | deficiency | |
| | | | | | (methylmalonic | |
| | | | | | acidemia); | |
| | | | | | Methylmalonic | |
| | | | | | Acidemia with | |
| | | | | | Homocystinuria | |
| MMADHC | 27249 | 0168288 | Q9H3L0 | 72 | cobalamin D | Organic acidemia |
| | | | | | deficiency | |
| | | | | | (methylmalonic | |
| | | | | | acidemia); | |
| | | | | | Methylmalonic | |
| | | | | | Acidemia with | |
| | | | | | Homocystinuria; | |
| | | | | | Homocystinuria; | |
| | | | | | Cobalamin C | |
| | | | | | Deficiency | |
| MCEE | 84693 | 0124370 | Q96PE7 | 73 | methylmalonic | Organic acidemia |
| | | | | | acidemia; | |
| | | | | | Cobalamin D | |
| | | | | | Deficiency | |
| PCCA | 5095 | 0175198 | P05165 | 74 | propionic acidemia | Organic acidemia |
| PCCB | 5096 | 0114054 | P05166 | 75 | propionic acidemia | Organic acidemia |
| UGT1A1 | 54658 | 0241635 | P22309, | 76 | Crigler-Najjar | |
| | | | Q5DT03 | | syndrome type 1 | |
| | | | | | Crigler-Najjar | |
| | | | | | syndrome type 2, | |
| | | | | | Gilbert syndrome | |
| ASS1 | 445 | 0130707 | P00966, | 77 | citrullinemia type I | Urea cycle disorder |
| | | | Q5T6L4 | | | |
| PAH | 5053 | 0171759 | A0A024RBG4, | 78 | Phenylalanine | Aminoacidopathy |
| | | | P00439 | | hydroxylase | |
| | | | | | deficiency | |
| PAL | | | | 79 | Phenylalanine | Aminoacidopathy |
| | | | | | hydroxylase | |
| | | | | | deficiency | |
| ATP8B1 | 5205 | 0081923 | O43520 | 80 | Progressive | |
| | | | | | familial | |
| | | | | | intrahepatic | |
| | | | | | cholestasis Type 1 | |
| ABCB11 | 8647 | 0073734, | O95342 | 81 | Progressive | |
| | | 0276582 | | | familial | |
| | | | | | intrahepatic | |
| | | | | | cholestasis Type 2; | |
| | | | | | Progressive | |
| | | | | | Familial | |
| | | | | | Intrahepatic | |
| | | | | | Cholestasis Type 3 | |
| ABCB4 | 5244 | 0005471 | P21439 | 82 | Progressive | |
| | | | | | familial | |
| | | | | | intrahepatic | |
| | | | | | cholestasis Type 3; | |
| | | | | | Progressive | |
| | | | | | Familial | |
| | | | | | Intrahepatic | |
| | | | | | Cholestasis Type 2 | |
| TJP2 | 9414 | 0119139 | B7Z2R3, | 83 | Progressive | |
| | | | Q9UDY2, | | familial | |
| | | | B7Z954 | | intrahepatic | |
| | | | | | cholestasis Type 4 | |
| IVD | 3712 | 0128928 | P26440, | 84 | isovaleric | Organic acidemia |
| | | | A0A0A0MT83 | | acidemia (IVD) | |
| GCDH | 2639 | 0105607 | A0A024R7F9, | 85 | glutaric acidemia | Organic acidemia |
| | | | Q92947 | | type I | |
| ETFA | 2108 | 0140374 | A0A0S2Z3L0, | 86 | multiple acyl-CoA | Organic acidemia |
| | | | P13804 | | dehydrogenase | |
| | | | | | deficiency (a.k.a. | |
| | | | | | glutaric aciduria | |
| | | | | | type II) | |
| ETFB | 2109 | 0105379 | P38117 | 87 | multiple acyl-CoA | Organic acidemia |
| | | | | | dehydrogenase | |
| | | | | | deficiency (a.k.a. | |
| | | | | | glutaric aciduria | |
| | | | | | type II) | |
| ETFDH | 2110 | 0171503 | B4DEQ0, | 88 | multiple acyl-CoA | Organic acidemia |
| | | | Q16134 | | dehydrogenase | |
| | | | | | deficiency (a.k.a. | |
| | | | | | glutaric aciduria | |
| | | | | | type II) | |
| ASL | 435 | 0126522 | A0A024RDL8, | 89 | argininosuccinate | Urea cycle disorder |
| | | | P04424, | | lyase (ASL) | |
| | | | A0A0S2Z316 | | deficiency | |
| D2HGDH | 728294 | 0180902 | B3KSR6, | 90 | D-2- | Organic acidemia |
| | | | B4E3K7, | | hydroxyglutaric | |
| | | | B5MCV2, | | aciduria type I | |
| | | | Q8N465 | | | |
| HMGCL | 3155 | 0117305 | P35914 | 91 | 3-hydroxy-3- | Organic academia |
| | | | | | methylglutaryl- | Urea cycle disorder |
| | | | | | CoA lyase | |
| | | | | | (3HMG) | |
| | | | | | deficiency | |
| MCCC1 | 56922 | 0078070 | Q68D27, | 92 | 3-methylcrotonyl- | Organic acidemia |
| | | | Q96RQ3, | | CoA carboxylase | |
| | | | A0A0S2Z693, | | (3MCC) | |
| | | | E9PHF7 | | deficiency | |
| MCCC2 | 64087 | 0131844, | A0A140VK29, | 93 | 3-methylcrotonyl- | Organic acidemia |
| | | 0281742, | Q9HCC0 | | CoA carboxylase | |
| | | 0275300 | | | (3MCC) | |
| | | | | | deficiency | |
| ABCD4 | 5826 | 0119688 | A0A024R6B9, | 94 | methylmalonic | Organic acidemia |
| | | | O14678, | | acidemia with | |
| | | | A0A024R6C8 | | homocystinuria | |
| HCFC1 | 3054 | 0172534 | P51610, | 95 | methylmalonic | Organic acidemia |
| | | | A6NEM2 | | acidemia with | |
| | | | | | homocystinuria | |
| LMBRD1 | 55788 | 0168216 | Q9NUN5 | 96 | methylmalonic | Organic acidemia |
| | | | | | acidemia with | |
| | | | | | homocystinuria | |
| ARG1 | 383 | 0118520 | P05089 | 97 | arginase (ARG1) | Urea cycle disorder |
| | | | | | deficiency | |
| SLC25A15 | 10166 | 0102743 | Q9Y619 | 98 | hyperammonemia- | Urea cycle disorder |
| | | | | | hyperornithinemia- | |
| | | | | | homocitrullinuria | |
| | | | | | (HHH) syndrome | |
| SLC25A13 | 10165 | 0004864 | Q9UJS0 | 99 | citrin deficiency | Urea cycle disorder |
| | | | | | citrullinemia type | |
| | | | | | II | |
| ALAD | 210 | 0148218 | P13716 | 100 | Acute Hepatic | Porphyria |
| | | | | | porphyria | |
| CPOX | 1371 | 0080819 | P36551 | 101 | Acute Hepatic | Porphyria |
| | | | | | porphyria | |
| HMBS | 3145 | 0256269, | P08397 | 102 | Acute Hepatic | Porphyria |
| | | 0281702 | | | porphyria; | |
| | | | | | Acute Intermittent | |
| | | | | | Porphyria | |
| PPOX | 5498 | 0143224 | P50336, | 103 | Acute Hepatic | Porphyria |
| | | | B4DY76 | | porphyria | |
| BTD | 686 | 0169814 | P43251 | 104 | Biotinidase | Organic acidemia |
| | | | | | Deficiency | |
| HLCS | 3141 | 0159267 | P50747 | 105 | Holocarboxylase | Organic acidemia |
| | | | | | Synthetase | |
| | | | | | Deficiency | |
| PC | 5091 | 0173599 | P11498 | 106 | Pyruvate | Urea cycle disorder |
| | | | A0A024R5C5 | | Carboxylase | |
| | | | | | Deficiency | |
| SLC7A7 | 9056 | 0155465 | Q9UM01 | 107 | Lysinuric Protein | Urea cycle disorder |
| | | | A0A0S2Z502 | | Intolerance | |
| CPT2 | 1376 | 0157184 | P23786 | 108 | Carnitine | Fatty Acid Oxidation |
| | | | A0A140VK13 | | Palmitoyltransferase | |
| | | | A0A1B0GTB8 | | Type II (CPT II) | |
| | | | | | Deficiency | |
| ACADM | 34 | 0117054 | P11310 | 109 | Medium Chain | Fatty Acid Oxidation |
| | | | A0A0S2Z366, | | Acyl-CoA | |
| | | | B7Z911, | | Dehydrogenase | |
| | | | Q5HYG7, | | (MCAD) | |
| | | | Q5T4U5, | | Deficiency | |
| | | | B4DJE7 | | | |
| ACADS | 35 | 0122971 | P16219 | 110 | Short Chain Acyl- | Fatty acid oxidation |
| | | | E5KSD5, | | CoA (SCAD) | |
| | | | B4DUH1, | | Dehydrogenase | |
| | | | E9PE82 | | Deficiency | |
| ACADVL | 37 | 0072778 | P49748 | 111 | Very Long Chain | Fatty acid oxidation |
| | | | B3KPA6 | | Acyl-CoA | |
| | | | | | Dehydrogenase | |
| | | | | | (VLCAD) | |
| | | | | | Deficiency | |
| AGL | 178 | 0162688 | P35573 | 112 | GSD III (Cori/ | Liver glycogen storage |
| | | | A0A0S2A4E4 | | Forbe Disease or | disorder |
| | | | | | Debrancher) | |
| G6PC | 2538 | 0131482 | P35575 | 113 | GSDIa (Von | Liver glycogen storage |
| | | | | | Gierke Disease) | disorder |
| GBE1 | 2632 | 0114480 | Q04446 | 114 | GSD IV (Andersen | Liver glycogen storage |
| | | | Q59ET0 | | Disease, Brancher | disorder |
| | | | | | Enzyme) | |
| PHKA1 | 5255 | 0067177 | P46020 | 115 | GSD IXa | |
| PHKA2 | 0044446 | 5256 | P46019 | 116 | GSD IXa | Liver glycogen storage |
| | 5256 | 0044446 | | | | disorder |
| PHKB | 5257 | 0102893 | Q93100 | 117 | GSD IXb | Liver glycogen storage |
| | | | | | | disorder |
| PHKG2 | 5261 | 0156873 | P15735 | 118 | GSD IXc | Liver glycogen storage |
| | | | | | | disorder |
| SLC37A4 | 2542 | 0281500 | O43826 | 119 | GSDIb. c, d | Liver glycogen storage |
| | | 0137700 | A0A024R3H9, | | | disorder |
| | | | A8K0S7, | | | |
| | | | A0A024R3L1, | | | |
| | | | B4DUH2 | | | |
| PMM2 | 5373 | 0140650 | O15305, | 120 | PMM2-CDG | Glycosylation disorder |
| | | | A0A0S2Z4J6, | | | |
| | | | Q59F02 | | | |
| CBS | 102724560, | 0160200 | P35520, | 121 | Cystathionine | Aminoacidopathy |
| | 875 | | P0DN79, | | Beta-Synthase | |
| | | | Q9NTF0, | | Deficiency | |
| | | | B7Z2D6 | | (Classic | |
| | | | | | Homocystinuria); | |
| | | | | | Homocystinuria | |
| FAH | 2184 | 0103876 | P16930 | 122 | Tyrosinemia Type | Aminoacidopathy |
| | | | | | I | |
| TAT | 6898 | 0198650 | P17735, | 123 | Tyrosinemia Type | Aminoacidopathy |
| | | | A0A140VKB7 | | II | |
| | | | | | Tyrosinemia Type | |
| | | | | | III | |
| GALT | 2592 | 0213930 | P07902, | 124 | Galactosemia | Carbohydrate disorder |
| | | | A0A0S2Z3Y7, | | due to galactose-1- | |
| | | | B2RAT6 | | phosphate | |
| | | | | | uridylyltranserase | |
| | | | | | (GALT) | |
| | | | | | deficiency | |
| GALK1 | 2584 | 0108479 | P51570 | 125 | Galactosemia | Carbohydrate disorder |
| GALE | 2582 | 0117308 | Q14376 | 126 | Galactosemia | Carbohydrate disorder |
| G6PD | 2539 | 0160211 | P11413 | 127 | Glucose-6- | Carbohydrate disorder |
| | | | | | Phosphate | |
| | | | | | Dehydrogenase | |
| | | | | | (G6PD) | |
| | | | | | Deficiency | |
| SLC3A1 | 6519 | 0138079 | Q07837, | 128 | Cystinuria | Aminoacidopathy |
| | | | A0A0S2Z4E1, | | | |
| | | | B8ZZK1 | | | |
| SLC7A9 | 11136 | 0021488 | P82251 | 129 | Cystinuria | Aminoacidopathy |
| MTHFR | 4524 | 0177000 | P42898, | 130 | Homocystinuria | Aminoacidopathy |
| | | | Q59GJ6, | | | |
| | | | Q81U67 | | | |
| MTR | 4548 | 0116984 | Q99707 | 131 | Homocystinuria | Aminoacidopathy |
| MTRR | 4552 | 0124275 | Q9UBK8 | 132 | Homocystinuria | Aminoacidopathy |
| ATP7B | 540 | 0123191 | P35670, | 133 | Wilson Disease | Metal transport disorder |
| | | | A0A024RDX3, | | Copper | |
| | | | B7ZLR4, | | Metabolism | |
| | | | B7ZLR3, | | Disorder | |
| | | | E7ET55 | | | |
| HPRT1 | 3251 | 0165704 | P00492, | 134 | Lesch-Nyhan | Purine Metabolism |
| | | | A0A140VJL3 | | Syndrome | Disorder |
| | | | | | Purine Metabolism | |
| | | | | | Disorder | |
| HJV | 148738 | 0168509 | Q6ZVN8 | 135 | Hemochromatosis, | |
| | | | | | Type 2A | |
| HAMP | 57817 | 0105697 | P81172 | 136 | Hemochromatosis | |
| | | | | | Type 2B: Primary | |
| | | | | | Hemochromatosis | |
| JAG1 | 182 | 0101384 | P78504, | 137 | Alagille Syndrome | |
| | | | Q99740 | | 1 | |
| TTR | 7276 | 0118271 | P02766, | 138 | Familial TTR | |
| | | | E9KL36 | | Amyloidoisis; | |
| | | | | | Familial amyloid | |
| | | | | | polyneuropathy | |
| AGXT | 189 | 0172482 | P21549 | 139 | Primary | |
| | | | | | Hyperoxaluria | |
| | | | | | Type I | |
| LIPA | 3988 | 0107798 | P38571 | 140 | Lysosomal Acid | Lyososomal storage |
| | | | A0A0A0MT32 | | Lipase Deficiency | disorder |
| SERPING1 | 710 | 0149131 | P05155, | 141 | Hereditary | |
| | | | A0A0S2Z4J1, | | Angioedma | |
| | | | B2R659, | | | |
| | | | E7EWE5, | | | |
| | | | B3KSP2, | | | |
| | | | G5E9S2 | | | |
| HSD17B4 | 3295 | 0133835 | P51659 | 142 | D-Bifunctional | Peroxisomal disorders |
| | | | | | Protein Deficiency | |
| | | | | | X-linked | |
| | | | | | Adrenoleukodystrophy | |
| UROD | 7389 | 0126088 | P06132 | 143 | Porphyria Cutanea | |
| | | | | | Tarda | |
| HFE | 3077 | 0010704 | Q30201 | 144 | Porphyria Cutanea | |
| | | | | | Tarda | |
| LPL | 4023 | 0175445 | P06858, | 145 | Lipoprotein Lipase | |
| | | | A0A1B1RVA9 | | Deficiency | |
| | | | | | (“hyperlipoproteinemia | |
| | | | | | type Ia; | |
| | | | | | Buerger-Gruetz | |
| | | | | | syndrome, or | |
| | | | | | Familial | |
| | | | | | hyperchylomicronemia) | |
| GRHPR | 9380 | 0137106 | Q9UBQ7 | 146 | Primary | |
| | | | | | Hyperoxaluria | |
| | | | | | Type II | |
| HOGA1 | 112817 | 0241935 | Q86XE5 | 147 | Primary | |
| | | | | | Hyperoxaluria | |
| | | | | | Type III | |
| LDLR | 3949 | 0130164 | P01130, | 148 | Homozygous | |
| | | | A0A024R7D5 | | Familial | |
| | | | | | Hypercholesterolemia | |
| ACAD8 | 27034 | 0151498 | Q9UKU7 | 149 | isobutyryl-CoA | Organic acidemia |
| | | | | | dehydrogenase | |
| | | | | | (IBD) deficiency | |
| ACADSB | 36 | 0196177 | P45954, | 150 | short-branched | Organic acidemia |
| | | | A0A0S2Z3P9 | | chain acyl-CoA | |
| | | | | | dehydrogenase | |
| | | | | | (SBCAD) | |
| | | | | | deficiency | |
| ACAT1 | 38 | 0075239 | A0A140VJX1, | 151 | beta-ketothiolase | Organic acidemia |
| | | | P24752 | | deficiency | |
| ACSF3 | 197322 | 0176715 | Q4G176, | 152 | combined malonic | Organic acidemia |
| | | | F5H5A1 | | and methylmalonic | |
| | | | | | aciduria | |
| ASPA | 443 | 0108381 | P45381, | 153 | Canavan disease | Organic acidemia |
| | | | Q6FH48 | | | |
| AUH | 549 | 0148090 | Q13825, | 154 | 3- | Organic acidemia |
| | | | B4DYI6 | | methylglutaconic | |
| | | | | | acidemia type I | |
| DNAJC19 | 131118 | 0205981 | Q96DA6, | 155 | dilated | Organic acidemia |
| | | | A0A0S2Z5X1 | | cardiomyopathy | |
| | | | | | with ataxia | |
| | | | | | syndrome (causes | |
| | | | | | 3- | |
| | | | | | methylglutaconic | |
| | | | | | aciduria) | |
| ETHE1 | 23474 | 0105755 | A0A0S2Z580, | 156 | ethylmalonic | Organic acidemia |
| | | | O95571, | | encephalopathy | |
| | | | A0A0S2Z5N8, | | | |
| | | | A0A0S2Z5B3, | | | |
| | | | B2RCZ7 | | | |
| FBP1 | 2203 | 0165140 | P09467, | 157 | fructose 1,6- | Organic acidemia |
| | | | Q2TU34 | | Bisphosphatase | |
| | | | | | deficiency | |
| FTCD | 10841 | 0160282, | O95954 | 158 | glutamate | Organic acidemia |
| | | 0281775 | | | formiminotransferase | |
| | | | | | deficiency | |
| | | | | | (FIGLU | |
| GSS | 2937 | 0100983 | P48637, | 159 | glutathione | Organic acidemia |
| | | | V9HWJ1 | | synthetase | |
| | | | | | deficiency | |
| HIBCH | 26275 | 0198130 | A0A140VJL0, | 160 | 3- | Organic acidemia |
| | | | Q6NVY1 | | hyroxyisobutyryl- | |
| | | | | | CoA hydrolase | |
| | | | | | deficiency | |
| IDH2 | 3418 | 0182054 | P48735, | 161 | D-2- | Organic acidemia |
| | | | B4DSZ6 | | hydroxyglutaric | |
| | | | | | aciduria type II | |
| L2HGDH | 79944 | 0087299 | Q9H9P8 | 162 | L-2- | Organic acidemia |
| | | | | | hydroxyglutaric | |
| | | | | | aciduria | |
| MLYCD | 23417 | 0103150 | O95822 | 163 | malonic acidemia | Organic acidemia |
| OPA3 | 80207 | 0125741 | Q9H6K4, | 164 | Costeff syndrome/ | Organic acidemia |
| | | | B4DK77 | | 3- | |
| | | | | | methylglutaconic | |
| | | | | | aciduria type III | |
| OPLAH | 26873 | 0178814 | O14841 | 165 | 5-oxoprolinase | Organic acidemia |
| | | | | | deficiency | |
| OXCT1 | 5019 | 0083720 | A0A024R040, | 166 | SCOT deficiency | Organic acidemia |
| | | | P55809 | | | |
| POLG | 5428 | 0140521 | E5KNU5, | 167 | 3- | Organic acidemia |
| | | | P54098 | | methylglutaconic | |
| | | | | | aciduria | |
| PPM1K | 152926 | 0163644 | Q8N3J5 | 168 | maple syrup urine | Organic acidemia |
| | | | | | disease (MSUD), | |
| | | | | | variant type | |
| SERAC1 | 84947 | 0122335 | Q96JX3 | 169 | Megdel Syndrome | Organic acidemia |
| SLC25A1 | 6576 | 0100075 | D9HTE9, | 170 | D,L-2- | Organic acidemia |
| | | | B4DP62, | | hydroxyglutaric | |
| | | | P53007 | | aciduria | |
| SUCLA2 | 8803 | 0136143 | E5KS60, | 171 | succinate-CoA | Organic acidemia |
| | | | Q9P2R7, | | ligase deficiency, | |
| | | | Q9Y4T0 | | methylmalonic | |
| | | | | | aciduria | |
| SUCLG1 | 8802 | 0163541 | P53597 | 172 | succinate-CoA | Organic acidemia |
| | | | | | ligase deficiency, | |
| | | | | | methylmalonic | |
| | | | | | aciduria | |
| TAZ | 6901 | 0102125 | A0A0S2Z4K0, | 173 | Barth syndrome | Organic acidemia |
| | | | Q16635, | | | |
| | | | A6XNE1, | | | |
| | | | A0A0S2Z4E6, | | | |
| | | | A0A0S2Z4K9, | | | |
| | | | A0A0S2Z4F4 | | | |
| AGK | 55750 | 0006530, | A4D1U5, | 174 | 3- | Organic acidemia |
| | | 0262327 | Q53H12 | | methylglutaconic | |
| | | | | | aciduria | |
| CLPB | 81570 | 0162129 | Q9H078, | 175 | 3- | Organic acidemia |
| | | | A0A140VK11 | | methylglutaconic | |
| | | | | | aciduria | |
| TMEM70 | 54968 | 0175606 | Q9BUB7 | 176 | 3- | Organic acidemia |
| | | | | | methylglutaconic | |
| | | | | | aciduria | |
| ALDH18A1 | 5832 | 0059573 | P54886 | 177 | ALDH18A1- | Urea cycle disorder |
| | | | | | related cutis laxa | |
| OAT | 4942 | 0065154 | A0A140VJQ4, | 178 | gyrate atrophy | Urea cycle disorder |
| | | | P04181 | | (OAT) | |
| CA5A | 763 | 0174990 | P35218 | 179 | carbonic | Urea cycle disorder |
| | | | | | anhydrase | |
| | | | | | deficiency | |
| GLUD1 | 2746 | 0148672 | P00367, | 180 | glutamate | Urea cycle disorder |
| | | | E9KL48 | | dehydrogenase | |
| | | | | | deficiency | |
| GLUL | 2752 | 0135821 | A8YXX4, | 181 | glutamine | Urea cycle disorder |
| | | | P15104 | | synthetase | |
| | | | | | deficienc | |
| UMPS | 7372 | 0114491 | A8K5J1, | 182 | Orotic Aciduria | Urea cycle disorder |
| | | | P11172 | | | |
| SLC22A5 | 6584 | 0197375 | O76082 | 183 | carnitine- | Fatty acid oxidation |
| | | | | | acylcarnitine | |
| | | | | | translocase | |
| | | | | | (CACT) | |
| | | | | | deficiency | |
| CPT1A | 1374 | 0110090 | P50416, | 184 | carnitine | Fatty acid oxidation |
| | | | A0A024R5F4, | | palmitoyltransferase | |
| | | | B2RAQ8, | | type I (CPT I) | |
| | | | Q8WZ48 | | deficiency | |
| HADHA | 3030 | 0084754 | E9KL44, | 185 | long chain 3- | Fatty acid oxidation |
| | | | P40939 | | hydroxyacyl-CoA | |
| | | | | | dehydrogenase | |
| | | | | | (LCHAD) | |
| | | | | | deficiency | |
| HADH | 3033 | 0138796 | Q16836, | 186 | medium/short | Fatty acid oxidation |
| | | | B3KTT6 | | chain acyl-CoA | |
| | | | | | dehydrogenase | |
| | | | | | (M/SCHAD) | |
| | | | | | deficiency | |
| SLC52A1 | 55065 | 0132517 | Q9NWF4 | 187 | Riboflavin | Fatty acid oxidation |
| | | | | | transporter | |
| | | | | | deficiency | |
| SLC52A2 | 79581 | 0185803 | Q9HAB3 | 188 | Riboflavin | Fatty acid oxidation |
| | | | | | transporter | |
| | | | | | deficiency | |
| SLC52A3 | 113278 | 0101276 | K0A6P4, | 189 | Riboflavin | Fatty acid oxidation |
| | | | Q9NQ40 | | transporter | |
| | | | | | deficiency | |
| HADHB | 3032 | 0138029 | P55084, | 190 | Trifunctional | Fatty acid oxidation |
| | | | F5GZQ3 | | protein deficiency | |
| GYS2 | 2998 | 0111713 | P54840 | 191 | GSD 0 (Glycogen | Liver glycogen storage |
| | | | | | synthase, liver | disorder |
| | | | | | isoform) | |
| PYGL | 5836 | 0100504 | P06737 | 192 | GSD VI (Hers | Liver glycogen storage |
| | | | | | disease) | disorder |
| SLC2A2 | 6514 | 0163581 | P11168, | 193 | Fanconi-Bickel | Liver glycogen storage |
| | | | Q6PAU8 | | syndrome | disorder |
| ALG1 | 56052 | 0033011 | Q9BT22 | 194 | ALG1-CDG | Glycosylation disorder |
| ALG2 | 85365 | 0119523 | A0A024R184, | 195 | ALG2-associated | Glycosylation disorder |
| | | | Q9H553 | | myasthenic | |
| | | | | | syndrome | |
| ALG3 | 10195 | 0214160 | Q92685, | 196 | ALG3-CDG | Glycosylation disorder |
| | | | C9J7S5 | | | |
| ALG6 | 29929 | 0088035 | Q9Y672 | 197 | ALG6-CDG | Glycosylation disorder |
| ALG8 | 79053 | 0159063 | Q9BVK2, | 198 | ALG8-CDG | Glycosylation disorder |
| | | | A0A024R5K5 | | | |
| ALG9 | 79796 | 0086848 | Q9H6U8 | 199 | ALG9-CDG | Glycosylation disorder |
| ALG11 | 440138 | 0253710 | Q2TAA5 | 200 | ALG11-CDG | Glycosylation disorder |
| ALG12 | 79087 | 0182858 | A0A024R4V6, | 201 | ALG12-CDG | Glycosylation disorder |
| | | | Q9BV10 | | | |
| ALG13 | 79868 | 0101901 | Q9NP73, | 202 | ALG13-CDG | Glycosylation disorder |
| | | | A0A087WX43, | | | |
| | | | A0A087WT15 | | | |
| ATP6V0A2 | 23545 | 0185344 | Q9Y487 | 203 | ATP6V0A2- | Glycosylation disorder |
| | | | | | associated cutis | |
| | | | | | laxa | |
| B3GLCT | 145173 | 0187676 | Q6Y288 | 204 | B3GLCT-CDG | Glycosylation disorder |
| CHST14 | 113189 | 0169105 | Q8NCH0 | 205 | CHST14-CDG | Glycosylation disorder |
| COG1 | 9382 | 0166685 | Q8WTW3 | 206 | COG1-CDG | Glycosylation disorder |
| COG2 | 22796 | 0135775 | Q14746, | 207 | COG2-CDG | Glycosylation disorder |
| | | | B1ALW7 | | | |
| COG4 | 25839 | 0103051 | A0A0A0MS45, | 208 | COG4-CDG | Glycosylation disorder |
| | | | Q8N8L9, | | | |
| | | | Q9H9E3, | | | |
| | | | J3KNI1 | | | |
| COG5 | 10466 | 0164597, | Q9UP83 | 209 | COG5-CDG | Glycosylation disorder |
| | | 0284369 | | | | |
| COG6 | 57511 | 0133103 | A0A140VJG7, | 210 | COG6-CDG | Glycosylation disorder |
| | | | Q9Y2V7, | | | |
| | | | A0A024RDW5 | | | |
| COG7 | 91949 | 0168434 | A0A0S2Z652, | 211 | COG7-CDG | Glycosylation disorder |
| | | | P83436 | | | |
| COG8 | 84342 | 0272617 | A0A024R6Z6, | 212 | COG8-CDG | Glycosylation disorder |
| | | | Q96MW5 | | | |
| DOLK | 22845 | 0175283 | A0A0S2Z597, | 213 | DOLK-CDG | Glycosylation disorder |
| | | | Q9UPQ8 | | | |
| DHDDS | 79947 | 0117682 | Q86SQ9 | 214 | DHDDS-CDG | Glycosylation disorder |
| DPAGT1 | 1798 | 0172269 | A0A024R3H8, | 215 | DPAGT1-CDG | Glycosylation disorder |
| | | | Q9H3H5 | | | |
| DPM1 | 8813 | 0000419 | O60762, | 216 | DPM1-CDG | Glycosylation disorder |
| | | | Q5QPK2, | | | |
| | | | A0A0S2Z4Y5 | | | |
| DPM2 | 8818 | 0136908 | O94777 | 217 | DPM2-CDG | Glycosylation disorder |
| DPM3 | 54344 | 0179085 | A0A140VJI4, | 218 | DPM3-CDG | Glycosylation disorder |
| | | | Q9P2X0, | | | |
| | | | Q86TM7 | | | |
| G6PC3 | 92579 | 0141349 | Q9BUM1 | 219 | Congenital | Glycosylation disorder |
| | | | | | neutropenia | |
| GFPT1 | 2673 | 0198380 | Q06210 | 220 | Congenital | Glycosylation disorder |
| | | | | | myasthenic | |
| | | | | | syndrome | |
| GMPPA | 29926 | 0144591 | A0A024R482, | 221 | GMPPA-CDG | Glycosylation disorder |
| | | | Q96IJ6 | | | |
| GMPPB | 29925 | 0173540 | Q9Y5P6 | 222 | Congenital | Glycosylation disorder |
| | | | | | muscular | |
| | | | | | dystrophy, | |
| | | | | | congenital | |
| | | | | | myasthenic | |
| | | | | | syndrome, and | |
| | | | | | dystroglycanopathy | |
| MAGT1 | 84061 | 0102158 | A0A087WU53, | 223 | MAGT1-CDG; X- | Glycosylation disorder |
| | | | Q9H0U3 | | linked | |
| | | | | | immunodeficiency | |
| | | | | | with magnesium | |
| | | | | | defect, Epstein- | |
| | | | | | Barr virus | |
| | | | | | infection and | |
| | | | | | neoplasia (XMEN) | |
| | | | | | syndrome | |
| MAN1B1 | 11253 | 0177239 | Q9UKM7 | 224 | MAN1B1-CDG | Glycosylation disorder |
| MGAT2 | 4247 | 0168282 | Q10469 | 225 | MGAT2-CDG | Glycosylation disorder |
| MOGS | 7841 | 0115275 | Q13724, | 226 | MOGS-CDG | Glycosylation disorder |
| | | | Q58F09 | | | |
| MPDU1 | 9526 | 0129255 | J3QW43, | 227 | MPDU1-CDG | Glycosylation disorder |
| | | | O75352, | | | |
| | | | A0A0S2Z4W8, | | | |
| | | | B4DLH7 | | | |
| MPI | 4351 | 0178802 | H3BPP3, | 228 | MPI-CDG | Glycosylation disorder |
| | | | Q8NHZ6, | | | |
| | | | B4DW50, | | | |
| | | | F5GX71, | | | |
| | | | P34949, | | | |
| | | | H3BPB8 | | | |
| NGLY1 | 55768 | 0151092 | Q96IV0 | 229 | NGLY1-CDG | Glycosylation disorder |
| PGM1 | 5236 | 0079739 | B7Z6C2, | 230 | PGM1-CDG | Glycosylation disorder |
| | | | P36871, | | | |
| | | | B4DDQ8 | | | |
| PGM3 | 5238 | 0013375 | O95394, | 231 | PGM3-CDG | Glycosylation disorder |
| | | | A0A087WT27 | | | |
| RFT1 | 91869 | 0163933 | Q96AA3 | 232 | RFT1-CDG | Glycosylation disorder |
| SEC23B | 10483 | 0101310 | Q15437, | 233 | SEC23B-CDG | Glycosylation disorder |
| | | | B4DJW8 | | | |
| SLC35A1 | 10559 | 0164414 | P78382 | 234 | SLC35A1-CDG | Glycosylation disorder |
| SLC35A2 | 7355 | 0102100 | P78381, | 235 | SLC35A2-CDG | Glycosylation disorder |
| | | | A6NFI1, | | | |
| | | | A6NKM8, | | | |
| | | | B4DE15 | | | |
| SLC35C1 | 55343 | 0181830 | Q96A29, | 236 | SLC35C1-CDG | Glycosylation disorder |
| | | | B3KQH0 | | | |
| SSR4 | 6748 | 0180879 | P51571 | 237 | SSR4-CDG | Glycosylation disorder |
| SRD5A3 | 79644 | 0128039 | Q9H8P0 | 238 | SRD5A3-CDG | Glycosylation disorder |
| TMEM165 | 55858 | 0134851 | Q9HC07 | 239 | TMEM165-CDG | Glycosylation disorder |
| TRIP11 | 9321 | 0100815 | Q15643 | 240 | TRIP11-CDG | Glycosylation disorder |
| TUSC3 | 7991 | 0104723 | Q13454 | 241 | TUSC3-CDG | Glycosylation disorder |
| ALG14 | 199857 | 0172339 | Q96F25 | 242 | ALG14-CDG | Glycosylation disorder |
| B4GALT1 | 2683 | 0086062 | P15291, | 243 | B4GALT1-CDG | Glycosylation disorder |
| | | | W6MEN3 | | | |
| DDOST | 1650 | 0244038 | A0A024RAD5, | 244 | DDOST-CDG | Glycosylation disorder |
| | | | P39656 | | | |
| NUS1 | 116150 | 0153989 | Q96E22 | 245 | NUS1-CDG | Glycosylation disorder |
| RPN2 | 6185 | 0118705 | P04844 | 246 | RPN2-CDG | Glycosylation disorder |
| SEC23A | 10484 | 0100934 | Q15436 | 247 | SEC23A-CDG | Glycosylation disorder |
| SLC35A3 | 23443 | 0117620 | Q9Y2D2, | 248 | SLC35A3-CDG | Glycosylation disorder |
| | | | A0A1W2PRT7, | | | |
| | | | A0A1W2PSD1, | | | |
| | | | A0A1W2PQL8 | | | |
| ST3GAL3 | 6487 | 0126091 | Q11203 | 249 | ST3GAL3-CDG | Glycosylation disorder |
| STT3A | 3703 | 0134910 | P46977 | 250 | STT3A-CDG | Glycosylation disorder |
| STT3B | 201595 | 0163527 | Q8TCJ2 | 251 | STT3B-CDG | Glycosylation disorder |
| AGA | 175 | 0038002 | P20933 | 252 | Aspartylglucosaminuria | Lyososomal storage |
| | | | | | | disorder |
| ARSA | 410 | 0100299 | A0A0C4DFZ2, | 253 | Metachromatic | Lyososomal storage |
| | | | B4DVI5, | | leukodystrophy | disorder |
| | | | P15289 | | | |
| ARSB | 411 | 0113273 | A0A024RAJ9, | 254 | Mucopolysaccharidosis | Lyososomal storage |
| | | | P15848, | | type VI | disorder |
| | | | A8K4A0 | | | |
| ASAH1 | 427 | 0104763 | A8K0B6, | 255 | Farber disease | Lyososomal storage |
| | | | Q13510, | | | disorder |
| | | | Q53H01 | | | |
| ATP13A2 | 23400 | 0159363 | Q8N4D4, | 256 | Neuronal ceroid | Lyososomal storage |
| | | | Q9NQ11, | | lipofuscinosis 12 | disorder |
| | | | Q8NBS1 | | (CLN12), Kufor- | |
| | | | | | Rakeb syndrome | |
| | | | | | (KRS) | |
| CLN3 | 1201 | 0188603, | A0A024QZB8, | 257 | Neuronal ceroid | Lyososomal storage |
| | | 0261832 | Q13286, | | lipofuscinosis 3 | disorder |
| | | | B4DMY6, | | (CLN3) | |
| | | | Q2TA70, | | | |
| | | | B4DFF3 | | | |
| CLN5 | 1203 | 0102805 | A0A024R644, | 258 | Neuronal ceroid | Lyososomal storage |
| | | | O75503 | | lipofuscinosis 5 | disorder |
| | | | | | (CLN5) | |
| CLN6 | 54982 | 0128973 | A0A024R601, | 259 | Neuronal ceroid | Lyososomal storage |
| | | | Q9NWW5 | | lipofuscinosis 6 | disorder |
| | | | | | (CLN6) | |
| CLN8 | 2055 | 0182372, | A0A024QZ57, | 260 | Neuronal ceroid | Lyososomal storage |
| | | 0278220 | Q9UBY8 | | lipofuscinosis 8 | disorder |
| | | | | | (CLN8) | |
| CTNS | 1497 | 0040531 | A0A0S2Z3I9, | 261 | cystinosis | Lyososomal storage |
| | | | O60931, | | | disorder |
| | | | A0A0S2Z3K3 | | | |
| CTSA | 5476 | 0064601 | P10619, | 262 | Galactosialidosis | Lyososomal storage |
| | | | X6R8A1, | | | disorder |
| | | | B4E324, | | | |
| | | | X6R5C5 | | | |
| CTSD | 1509 | 0117984 | P07339, | 263 | Neuronal ceroid | Lyososomal storage |
| | | | V9HWI3 | | lipofuscinosis 10 | disorder |
| | | | | | (CLN10) | |
| CTSF | 8722 | 0174080 | Q9UBX1 | 264 | Neuronal ceroid | Lyososomal storage |
| | | | | | lipofuscinosis 13 | disorder |
| | | | | | (CLN13) | |
| CTSK | 1513 | 0143387 | P43235 | 265 | Pycnodysostosis | Lyososomal storage |
| | | | | | | disorder |
| DNAJC5 | 80331 | 0101152 | Q6AHX3, | 266 | Neuronal ceroid | Lyososomal storage |
| | | | Q9H3Z4 | | lipofuscinosis 4 | disorder |
| | | | | | (CLN4) | |
| FUCA1 | 2517 | 0179163 | P04066, | 267 | Fucosidosis | Lyososomal storage |
| | | | B5MDC5 | | | disorder |
| GAA | 2548 | 0171298 | P10253 | 268 | Pompe disease | Lyososomal storage |
| | | | | | | disorder |
| GALC | 2581 | 0054983 | A0A0A0MQV0, | 269 | Krabbe disease | Lyososomal storage |
| | | | P54803 | | | disorder |
| GALNS | 2588 | 0141012 | P34059, | 270 | Mucopolysaccharidosis | Lyososomal storage |
| | | | Q96I49, | | type IVa | disorder |
| | | | Q6YL38 | | | |
| GLA | 2717 | 0102393 | P06280, | 271 | Fabry disease | Lyososomal storage |
| | | | Q53Y83 | | | disorder |
| GLB1 | 2720 | 0170266 | P16278, | 272 | GM1 | Lyososomal storage |
| | | | B7Z6Q5 | | gangliosidosis, | disorder |
| | | | | | Mucopolysaccharidosis | |
| | | | | | IVb | |
| GM2A | 2760 | 0196743 | P17900 | 273 | GM2- | Lyososomal storage |
| | | | | | gangliosidosis, AB | disorder |
| | | | | | variant | |
| GNPTAB | 79158 | 0111670 | Q3T906 | 274 | Mucolipidosis type | Lyososomal storage |
| | | | | | II alpha/beta, | disorder |
| | | | | | Mucolipidosis III | |
| | | | | | alpha/beta | |
| GNPTG | 84572 | 0090581 | Q9UJJ9 | 275 | Mucolipidosis III | Lyososomal storage |
| | | | | | gamma | disorder |
| GNS | 2799 | 0135677 | A0A024RBC5, | 276 | Mucopolysaccharidosis | Lyososomal storage |
| | | | P15586, | | type IIID | disorder |
| | | | Q7Z3X3 | | | |
| GRN | 2896 | 0030582 | P28799 | 277 | Neuronal ceroid | Lyososomal storage |
| | | | | | lipofuscinosis 11 | disorder |
| | | | | | (CLN11), | |
| | | | | | frontotemporal | |
| | | | | | dementia | |
| GUSB | 2990 | 0169919 | P08236 | 278 | Mucopolysaccharidosis | Lyososomal storage |
| | | | | | type VII | disorder |
| HEXA | 3073 | 0213614 | A0A0S2Z3W3, | 279 | Tay-Sachs disease | Lyososomal storage |
| | | | P06865, | | | disorder |
| | | | B4DVA7, | | | |
| | | | H3BP20 | | | |
| HEXB | 3074 | 0049860 | A0A024RAJ6, | 280 | Sandhoff diseaase | Lyososomal storage |
| | | | P07686, | | | disorder |
| | | | Q5URX0 | | | |
| HGSNAT | 138050 | 0165102 | Q68CP4, | 281 | Mucopolysaccharidosis | Lyososomal storage |
| | | | Q8IVU6 | | type IIIC | disorder |
| HYAL1 | 3373 | 0114378 | A0A024R2X3, | 282 | Mucopolysaccharidosis | Lyososomal storage |
| | | | QI2794, | | type IX | disorder |
| | | | B3KUI5, | | | |
| | | | A0A0S2Z3Q0 | | | |
| IDS | 3423 | 0010404 | P22304, | 283 | Mucopolysaccharidosis | Lyososomal storage |
| | | | B4DGD7 | | type II | disorder |
| IDUA | 3425 | 0127415 | P35475 | 284 | Mucopolysaccharidosis | Lyososomal storage |
| | | | | | type I | disorder |
| KCTD7 | 154881 | 0243335 | Q96MP8, | 285 | Neuronal ceroid | Lyososomal storage |
| | | | A0A024RDN7 | | lipofuscinosis 14 | disorder |
| | | | | | (CLN14) | |
| LAMP2 | 3920 | 0005893 | P13473 | 286 | Danon disease | Lyososomal storage |
| | | | | | | disorder |
| MAN2B1 | 4125 | 0104774 | O00754, | 287 | alpha- | Lyososomal storage |
| | | | A8K6A7 | | mannosidosis | disorder |
| MANBA | 4126 | 0109323 | O00462 | 288 | beta-mannosidosis | Lyososomal storage |
| | | | | | | disorder |
| MCOLN1 | 57192 | 0090674 | Q9GZU1 | 289 | Mucolipidosis type | Lyososomal storage |
| | | | | | IV | disorder |
| MFSD8 | 256471 | 0164073 | Q8NHS3 | 290 | Neuronal ceroid | Lyososomal storage |
| | | | | | lipofuscinosis 7 | disorder |
| | | | | | (CLN7) | |
| NAGA | 4668 | 0198951 | A0A024R1Q5, | 291 | Schindler disease | Lyososomal storage |
| | | | P17050 | | | disorder |
| NAGLU | 4669 | 0108784 | A0A140VJE4, | 292 | Mucopolysaccharidosis | Lyososomal storage |
| | | | P54802 | | IIIB | disorder |
| NEU1 | 4758 | 0204386, | Q5JQI0, | 293 | Mucolipidosis type | Lyososomal storage |
| | | 0227315, | Q99519 | | I, Sialidosis I | disorder |
| | | 0227129, | | | | |
| | | 0223957, | | | | |
| | | 0234846, | | | | |
| | | 0184494, | | | | |
| | | 0228691, | | | | |
| | | 0234343 | | | | |
| NPC1 | 4864 | 0141458 | O15118 | 294 | Niemann-Pick | Lyososomal storage |
| | | | | | type C | disorder |
| NPC2 | 10577 | 0119655 | A0A024R6C0, | 295 | Niemann-Pick | Lyososomal storage |
| | | | P61916, | | type C | disorder |
| | | | G3V3E8 | | | |
| SGSH | 6448 | 0181523 | P51688 | 296 | Mucopolysaccharidosis | Lyososomal storage |
| | | | | | IIIA | disorder |
| PPT1 | 5538 | 0131238 | P50897 | 297 | Neuronal ceroid | Lyososomal storage |
| | | | | | lipofuscinosis 1 | disorder |
| | | | | | (CLN1) | |
| PSAP | 5660 | 0197746 | P07602, | 298 | Prosaposin | Lyososomal storage |
| | | | A0A024QZQ2 | | deficiency, SapA | disorder |
| | | | | | deficiency (Krabbe | |
| | | | | | variant), SapB | |
| | | | | | deficiency | |
| | | | | | (MLD variant), | |
| | | | | | SapC deficiency | |
| | | | | | (Gaucher variant) | |
| SLC17A5 | 26503 | 0119899 | Q9NRA2 | 299 | Infantile sialic acid | Lyososomal storage |
| | | | | | storage disease, | disorder |
| | | | | | Salla disease | |
| SMPD1 | 6609 | 0166311 | P17405, | 300 | Niemann Pick | Lyososomal storage |
| | | | Q59EN6, | | types A and B | disorder |
| | | | E9LUE8, | | | |
| | | | Q8IUN0, | | | |
| | | | E9LUE9 | | | |
| SUMF1 | 285362 | 0144455 | Q8NBK3 | 301 | Multiple sulfatase | Lyososomal storage |
| | | | | | deficiency | disorder |
| TPP1 | 1200 | 0166340 | O14773 | 302 | Neuronal ceroid | Lyososomal storage |
| | | | | | lipofuscinosis 2 | disorder |
| | | | | | (CLN2) | |
| AHCY | 191 | 0101444 | P23526, | 303 | Hypermethioninemia | Aminoacidophaty |
| | | | Q1RMG2 | | | |
| GNMT | 27232 | 0124713 | A0A0S2Z5F2, | 304 | Hypermethioninemia | Aminoacidophaty |
| | | | Q14749, | | | |
| | | | V9HW60 | | | |
| MAT1A | 4143 | 0151224 | Q00266 | 305 | Hypermethioninemia | Aminoacidophaty |
| GCH1 | 2643 | 0131979 | A0A024R642, | 306 | BH4 cofactor | Aminoacidophaty |
| | | | P30793, | | deficiency | |
| | | | Q8IZH9 | | | |
| PCBD1 | 5092 | 0166228 | P61457 | 307 | BH4 cofactor | Aminoacidophaty |
| | | | | | deficiency | |
| PTS | 5805 | 0150787 | Q03393 | 308 | BH4 cofactor | Aminoacidophaty |
| | | | | | deficiency | |
| QDPR | 5860 | 0151552 | A0A140VKA9, | 309 | BH4 cofactor | Aminoacidophaty |
| | | | P09417 | | deficiency | |
| SPR | 6697 | 0116096 | P35270 | 310 | BH4 cofactor | Aminoacidophaty |
| | | | | | deficiency | |
| DNAJC12 | 56521 | 0108176 | Q6IAH1, | 311 | Phenylalanine, | Aminoacidophaty |
| | | | Q9UKB3 | | tyrosine, and | |
| | | | | | tryptophan | |
| | | | | | hydroxylases heat | |
| | | | | | shock | |
| | | | | | co-chaperone | |
| | | | | | deficiency | |
| ALDH4A1 | 8659 | 0159423 | P30038, | 312 | Hyperprolinemia | Aminoacidophaty |
| | | | A0A024RAD8 | | | |
| PRODH | 5625 | 0100033 | O43272 | 313 | Hyperprolinemia | Aminoacidophaty |
| HPD | 3242 | 0158104 | P32754 | 314 | Tyrosinemia type | Aminoacidophaty |
| | | | | | II | |
| GBA | 2629 | 0177628, | A0A068F658, | 315 | Gaucher disease | |
| | | 0262446 | P04062, | | | |
| | | | B7Z6S9 | | | |
| HGD | 3081 | 0113924 | Q93099, | 316 | Alkaptonuria | |
| | | | B3KW64 | | | |
| AMN | 81693 | 0166126 | Q9BXJ7, | 317 | Combined | Organic acidemia |
| | | | B3KP64 | | Methylmalonic | |
| | | | | | Acidemia and | |
| | | | | | Homocystinuria | |
| CD320 | 51293 | 0167775 | Q9NPF0 | 318 | Combined | Organic acidemia |
| | | | | | Methylmalonic | |
| | | | | | Acidemia and | |
| | | | | | Homocystinuria | |
| CUBN | 8029 | 0107611 | O60494 | 319 | Combined | Organic acidemia |
| | | | | | Methylmalonic | |
| | | | | | Acidemia and | |
| | | | | | Homocystinuria | |
| GIF | 2694 | 0134812 | P27352 | 320 | Combined | Organic acidemia |
| | | | | | Methylmalonic | |
| | | | | | Acidemia and | |
| | | | | | Homocystinuria | |
| TCN1 | 6947 | 0134827 | P20061 | 321 | Combined | Organic acidemia |
| | | | | | Methylmalonic | |
| | | | | | Acidemia and | |
| | | | | | Homocystinuria | |
| TCN2 | 6948 | 0185339 | P20062 | 322 | Combined | Organic acidemia |
| | | | | | Methylmalonic | |
| | | | | | Acidemia and | |
| | | | | | Homocystinuria | |
| PREPL | 9581 | 0138078 | Q4J6C6 | 323 | Cystinuria | Aminoacidophaty |
| PHGDH | 26227 | 0092621 | O43175 | 324 | Disorders of | Aminoacidophaty |
| | | | | | Serine | |
| | | | | | Biosynthesis | |
| PSAT1 | 29968 | 0135069 | A0A024R280, | 325 | Disorders of | Aminoacidophaty |
| | | | Q9Y617, | | Serine | |
| | | | A0A024R222 | | Biosynthesis | |
| PSPH | 5723 | 0146733 | A0A024RDL3, | 326 | Disorders of | Aminoacidophaty |
| | | | P78330 | | Serine | |
| | | | | | Biosynthesis | |
| AMT | 275 | 0145020 | A0A024R2U7, | 327 | Glycine | Aminoacidophaty |
| | | | P48728 | | Encephalopathy | |
| GCSH | 2653 | 0140905 | P23434 | 328 | Glycine | Aminoacidophaty |
| | | | | | Encephalopathy | |
| GLDC | 2731 | 0178445 | P23378 | 329 | Glycine | Aminoacidophaty |
| | | | | | Encephalopathy | |
| LIAS | 11019 | 0121897 | O43766, | 330 | Glycine | Aminoacidophaty |
| | | | Q6P5Q6, | | Encephalopathy | |
| | | | B4E0L7, | | | |
| | | | A0A024R9W0, | | | |
| | | | A0A1W2PQE9, | | | |
| | | | A0A1X7SBR7 | | | |
| NFU1 | 27247 | 0169599 | Q9UMS0 | 331 | Glycine | Aminoacidophaty |
| | | | | | Encephalopathy | |
| SLC6A9 | 6536 | 0196517 | P48067, | 332 | Glycine | Aminoacidophaty |
| | | | B7Z3W8, | | Encephalopathy | |
| | | | B7Z589 | | | |
| SLC2A1 | 6513 | 0117394 | P11166, | 333 | Glucose | Carbohydrate disorder |
| | | | Q59GX2 | | Transporter Type 1 | |
| | | | | | Deficiency | |
| ATP7A | 538 | 0165240 | B4DRW0, | 334 | ATP7A-Related | Metal transport disorder |
| | | | Q04656, | | Disorders | |
| | | | Q762B6 | | Copper | |
| | | | | | Metabolism | |
| | | | | | Disorder | |
| AP1S1 | 1174 | 0106367 | A0A024QYT6, | 335 | Copper | Metal transport disorder |
| | | | P61966 | | Metabolism | |
| | | | | | Disorder | |
| CP | 1356 | 0047457 | A5PL27, | 336 | Copper | Metal transport disorder |
| | | | P00450 | | Metabolism | |
| | | | | | Disorder | |
| SLC33A1 | 9197 | 0169359 | O00400 | 337 | Copper | Metal transport disorder |
| | | | | | Metabolism | |
| | | | | | Disorder | |
| PEX7 | 5191 | 0112357 | O00628, | 338 | Adult Refsum | Peroxisomal disorders |
| | | | Q6FGN1 | | Disease | |
| | | | | | Rhizomelic | |
| | | | | | Chondrodysplasia | |
| | | | | | Punctata Spectrum | |
| PHYH | 5264 | 0107537 | O14832 | 339 | Adult Refsum | Peroxisomal disorders |
| | | | | | Disease | |
| AGPS | 8540 | 0018510 | O00116, | 340 | Rhizomelic | Peroxisomal disorders |
| | | | B7Z3Q4 | | Chondrodysplasia | |
| | | | | | Punctata Spectrum | |
| GNPAT | 8443 | 0116906 | O15228 | 341 | Rhizomelic | Peroxisomal disorders |
| | | | | | Chondrodysplasia | |
| | | | | | Punctata Spectrum | |
| ABCD1 | 215 | 0101986 | P33897 | 342 | X-linked | Peroxisomal disorders |
| | | | | | Adrenoleukodystrophy | |
| ACOX1 | 51 | 0161533 | Q15067 | 343 | X-linked | Peroxisomal disorders |
| | | | | | Adrenoleukodystrophy | |
| PEX1 | 5189 | 0127980 | O43933, | 344 | X-linked | Peroxisomal disorders |
| | | | A0A0C4DG33, | | Adrenoleukodystrophy | |
| | | | B4DER6 | | | |
| PEX2 | 5828 | 0164751 | P28328 | 345 | X-linked | Peroxisomal disorders |
| | | | | | Adrenoleukodystrophy | |
| PEX3 | 8504 | 0034693 | P56589 | 346 | X-linked | Peroxisomal disorders |
| | | | | | Adrenoleukodystrophy | |
| PEX5 | 5830 | 0139197 | A0A0S2Z480, | 347 | X-linked | Peroxisomal disorders |
| | | | P50542, | | Adrenoleukodystrophy | |
| | | | B4DR50, | | | |
| | | | A0A0S2Z4F3, | | | |
| | | | A0A0S2Z4H1, | | | |
| | | | B4E0T2 | | | |
| PEX6 | 5190 | 0124587 | A0A024RD09, | 348 | X-linked | Peroxisomal disorders |
| | | | Q13608 | | Adrenoleukodystrophy | |
| PEX10 | 5192 | 0157911 | A0A024R068, | 349 | X-linked | Peroxisomal disorders |
| | | | O60683, | | Adrenoleukodystrophy | |
| | | | A0A024R0A4 | | | |
| PEX12 | 5193 | 0108733 | O00623 | 350 | X-linked | Peroxisomal disorders |
| | | | | | Adrenoleukodystrophy | |
| PEX13 | 5194 | 0162928 | Q92968 | 351 | X-linked | Peroxisomal disorders |
| | | | | | Adrenoleukodystrophy | |
| PEX14 | 5195 | 0142655 | O75381 | 352 | X-linked | Peroxisomal disorders |
| | | | | | Adrenoleukodystrophy | |
| PEX16 | 9409 | 0121680 | Q9Y5Y5 | 353 | X-linked | Peroxisomal disorders |
| | | | | | Adrenoleukodystrophy | |
| PEX19 | 5824 | 0162735 | P40855, | 354 | X-linked | Peroxisomal disorders |
| | | | A0A0S2Z497 | | Adrenoleukodystrophy | |
| PEX26 | 55670 | 0215193 | A0A024R100, | 355 | X-linked | Peroxisomal disorders |
| | | | Q7Z412, | | Adrenoleukodystrophy | |
| | | | A0A0S2Z5M7, | | | |
| | | | Q7Z2D7 | | | |
| AMACR | 23600 | 0242110 | Q9UHK6 | 356 | Zellweger | Peroxisomal disorders |
| | | | | | Spectrum Disorder | |
| ADA | 100 | 0196839 | A0A0S2Z381, | 357 | Purine Metabolism | Purine Metabolism |
| | | | P00813, | | Disorder | Disorder |
| | | | F5GWI4 | | | |
| ADSL | 158 | 0239900 | P30566, | 358 | Purine Metabolism | Purine Metabolism |
| | | | X5D8S6, | | Disorder | Disorder |
| | | | X5D7W4, | | | |
| | | | A0A1B0GWJ0 | | | |
| AMPD1 | 270 | 0116748 | P23109 | 359 | Purine Metabolism | Purine Metabolism |
| | | | | | Disorder | Disorder |
| GPHN | 10243 | 0171723 | Q9NQX3 | 360 | Purine Metabolism | Purine Metabolism |
| | | | | | Disorder | Disorder |
| MOCOS | 55034 | 0075643 | Q96EN8 | 361 | Purine Metabolism | Purine Metabolism |
| | | | | | Disorder | Disorder |
| MOCS1 | 4337 | 0124615 | A0A024RD17, | 362 | Purine Metabolism | Purine Metabolism |
| | | | Q9NZB8 | | Disorder | Disorder |
| PNP | 4860 | 0198805 | P00491, | 363 | Purine Metabolism | Purine Metabolism |
| | | | V9HWH6 | | Disorder | Disorder |
| XDH | 7498 | 0158125 | P47989 | 364 | Purine Metabolism | Purine Metabolism |
| | | | | | Disorder | Disorder |
| SUOX | 6821 | 0139531 | A0A024RB79, | 365 | Purine Metabolism | Purine Metabolism |
| | | | P51687 | | Disorder | Disorder |
| OGDH | 4967 | 0105953 | A0A140VJQ5, | 366 | 2-Ketoglutarate | PYRUVATE |
| | | | Q02218, | | Dehydrogenase | METABOLISM AND |
| | | | B4E3E9, | | Deficiency | TRICARBOXYLIC ACID |
| | | | E9PCR7, | | | CYCLE DEFECT |
| | | | E9PDF2 | | | |
| SLC25A19 | 60386 | 0125454 | Q5JPC1, | 367 | 2-Ketoglutarate | PYRUVATE |
| | | | Q9HC21 | | Dehydrogenase | METABOLISM AND |
| | | | | | Deficiency | TRICARBOXYLIC ACID |
| | | | | | | CYCLE DEFECT |
| DHTKD1 | 55526 | 0181192 | Q96HY7 | 368 | 2-Ketoglutarate | PYRUVATE |
| | | | | | Dehydrogenase | METABOLISM AND |
| | | | | | Deficiency | TRICARBOXYLIC ACID |
| | | | | | | CYCLE DEFECT |
| SLC13A5 | 284111 | 0141485 | Q68D44, | 369 | Citrate Transporter | PYRUVATE |
| | | | Q86YT5 | | Deficiency | METABOLISM AND |
| | | | | | | TRICARBOXYLIC ACID |
| | | | | | | CYCLE DEFECT |
| FH | 2271 | 0091483 | A0A0S2Z4C3, | 370 | Fumarase | PYRUVATE |
| | | | P07954 | | Deficiency | METABOLISM AND |
| | | | | | | TRICARBOXYLIC ACID |
| | | | | | | CYCLE DEFECT |
| DLAT | 1737 | 0150768 | P10515, | 371 | Pyruvate | PYRUVATE |
| | | | Q86YI5 | | Dehydrogenase | METABOLISM AND |
| | | | | | Deficiency | TRICARBOXYLIC ACID |
| | | | | | | CYCLE DEFECT |
| MPC1 | 51660 | 0060762 | Q5TI65, | 372 | Pyruvate | PYRUVATE |
| | | | Q9Y5U8 | | Dehydrogenase | METABOLISM AND |
| | | | | | Deficiency | TRICARBOXYLIC ACID |
| | | | | | | CYCLE DEFECT |
| PDHA1 | 5160 | 0131828 | A0A024RBX9, | 373 | Pyruvate | PYRUVATE |
| | | | P08559 | | Dehydrogenase | METABOLISM AND |
| | | | | | Deficiency | TRICARBOXYLIC ACID |
| | | | | | | CYCLE DEFECT |
| PDHB | 5162 | 0168291 | P11177 | 374 | Pyruvate | PYRUVATE |
| | | | | | Dehydrogenase | METABOLISM AND |
| | | | | | Deficiency | TRICARBOXYLIC ACID |
| | | | | | | CYCLE DEFECT |
| PDHX | 8050 | 0110435 | O00330 | 375 | Pyruvate | PYRUVATE |
| | | | | | Dehydrogenase | METABOLISM AND |
| | | | | | Deficiency | TRICARBOXYLIC ACID |
| | | | | | | CYCLE DEFECT |
| PDP1 | 54704 | 0164951 | Q9P0J1, | 376 | Pyruvate | PYRUVATE |
| | | | Q6P1N1, | | Dehydrogenase | METABOLISM AND |
| | | | A0A024R9C0 | | Deficiency | TRICARBOXYLIC ACID |
| | | | | | | CYCLE DEFECT |
| ABCC2 | 1244 | 0023839 | Q92887 | 377 | Dubin-Johnson | |
| | | | | | syndrome | |
| SLCO1B1 | 10599 | 0134538 | A0A024RAU7, | 378 | Rotor Syndrome | |
| | | | Q05CV5, | | | |
| | | | Q9Y6L6 | | | |
| SLCO1B3 | 28234 | 0111700 | B3KP78, | 379 | Rotor Syndrome | |
| | | | Q9NPD5 | | | |
| HFE2 | 148738 | 0168509 | Q6ZVN8, | 380 | Hemochromatosis, | |
| | | | A8K466, | | type 2A | |
| | | | A0A024R4F5 | | | |
| ADAMTS13 | 11093 | 0160323, | Q76LX8 | 381 | Congenital | |
| | | 0281244 | | | thrombotic | |
| | | | | | thrombocytopenic | |
| | | | | | purpura due to | |
| | | | | | ADAMTS-13 | |
| | | | | | deficiency | |
| PYGM | 5837 | 0068976 | P11217 | 382 | McArdle's Disease | |
| COL1A2 | 1278 | 0164692 | A0A0S2Z3H5, | 383 | Ehlers-Danlos | |
| | | | P08123 | | syndrome, cardiac | |
| | | | | | valvular type | |
| TNFRSF11B | 4982 | 0164761 | O00300 | 384 | Juvenile Paget's | |
| | | | | | disease | |
| TSC1 | 7248 | 0165699 | Q86WV8, | 385 | Tuberous sclerosis | |
| | | | Q92574, | | | |
| | | | X5D9D2, | | | |
| | | | Q32NF0 | | | |
| TSC2 | 7249 | 0103197 | P49815, | 386 | Tuberous sclerosis | |
| | | | X5D7Q2, | | | |
| | | | B3KWH7, | | | |
| | | | Q5HYF7, | | | |
| | | | H3BMQ0, | | | |
| | | | X5D2U8 | | | |
| DHCR7 | 1717 | 0172893 | A0A024R5F7, | 387 | Smith-Lemli-Opitz | |
| | | | Q9UBM7 | | Syndrome | |
| PGK1 | 5230 | 0102144 | P00558, | 388 | D- | |
| | | | V9HWF4 | | glycericacidemia | |
| VLDLR | 7436 | 0147852 | P98155, | 389 | Dysequilibrium | |
| | | | Q5VVF5 | | syndrome | |
| KYNU | 8942 | 0115919 | Q16719 | 390 | Encephalopathy | |
| | | | | | due to | |
| | | | | | hydroxykynureninuria | |
| F5 | 2153 | 0198734 | P12259 | 391 | Factor V | |
| | | | | | deficiency | |
| C3 | 718 | 0125730 | B4DR57, | 392 | Atypical hemolytic | |
| | | | P01024, | | uremic syndrome | |
| | | | V9HWA9 | | with C3 anomaly | |
| COL4A1 | 1282 | 0187498 | A5PKV2, | 393 | Autosomal | |
| | | | F5H5K0, | | dominant familial | |
| | | | P02462 | | hematuria - retinal | |
| | | | | | arteriolar | |
| | | | | | tortuosity - | |
| | | | | | contractures | |
| CFH | 3075 | 0000971 | A0A024R962, | 394 | Atypical hemolytic | |
| | | | P08603, | | uremic syndrome | |
| | | | A0A0D9SG88 | | | |
| SLC12A2 | 6558 | 0064651 | P55011, | 395 | Bartter syndrome | |
| | | | Q53ZR1, | | type I (neonatal) | |
| | | | B7ZM24 | | | |
| GK | 2710 | 0198814 | B4DH54, | 396 | Glycerol kinase | |
| | | | P32189 | | deficiency | |
| SFTPC | 6440 | 0168484 | A0A0A0MTC9, | 397 | Chronic | |
| | | | P11686, | | respiratory distress | |
| | | | A0A0S2Z4Q0, | | with surfactant | |
| | | | E5RI64 | | metabolism | |
| | | | | | deficiency | |
| CRTAP | 10491 | 0170275 | O75718 | 398 | Osteogenesis | |
| | | | | | Imperfecta VII | |
| P3H1 | 64175 | 0117385 | Q32P28 | 399 | Osteogenesis | |
| | | | | | Imperfecta VIII | |
| COL7A1 | 1294 | 0114270 | Q02388, | 400 | Autosomal | |
| | | | Q59F16 | | recessive | |
| | | | | | dystrophic | |
| | | | | | epidermolysis | |
| | | | | | bullosa | |
| PKLR | 5313 | 0143627 | P30613 | 401 | Pyruvate Kinase | |
| | | | | | deficiency | |
| TALDO1 | 6888 | 0177156 | A0A140VK56, | 402 | Transaldolase | |
| | | | P37837 | | deficiency | |
| TF | 7018 | 0091513 | A0PJA6, | 403 | Atransferrinemia | |
| | | | P02787, | | (familial | |
| | | | Q06AH7 | | hypotransferrinemia) | |
| EPCAM | 4072 | 0119888 | P16422 | 404 | Intestinal epithelial | |
| | | | | | dysplasia | |
| VHL | 7428 | 0134086 | A0A024R2F2, | 405 | Familial | |
| | | | P40337, | | erythrocytosis type | |
| | | | A0A0S2Z4K1 | | 2; von Hippel | |
| | | | | | Lindau disease | |
| GC | 2638 | 0145321 | P02774 | 406 | Vitamin D | |
| | | | | | deficiency | |
| SERPINA1 | 5265 | 0197249, | E9KL23, | 407 | Alpha-1 | |
| | | 0277377 | P01009 | | antitrypsin | |
| | | | | | deficiency | |
| ABCC6 | 368 | 0091262, | O95255 | 408 | Pseudoxanthoma | |
| | | 0275331 | | | elasticum | |
| F8 | 2157 | 0185010 | P00451 | 409 | Hemophilia A | |
| F9 | 2158 | 0101981 | P00740 | 410 | Hemophilia B | |
| ApoB | 338 | 0084674 | P04114 | 411 | Familial | |
| | | | | | hypercholesterolemia | |
| PCSK9 | 255738 | 0169174 | Q8NBP7 | 412 | Familial | |
| | | | | | hypercholesterolemia | |
| LDLRAP1 | 26119 | 0157978 | B3KR97, | 413 | Familial | |
| | | | Q5SW96 | | hypercholesterolemia | |
| ABCG5 | 64240 | 0138075 | Q9H222 | 414 | Sitosterolemia | |
| ABCG8 | 64241 | 0143921 | Q9H221 | 415 | Sitosterolemia | |
| LCAT | 3931 | 0213398 | A0A140VK24, | 416 | Lecithin | |
| | | | P04180 | | cholesterol | |
| | | | | | acyltransferase | |
| | | | | | deficiency | |
| SPINK5 | 11005 | 0133710 | Q9NQ38 | 417 | Netherton | |
| | | | | | syndrome | |
| GNE | 10020 | 0159921 | Q9Y223 | 418 | Inclusion body | |
| | | | | | myopathy 2 | |

In some embodiments, the targeted lipid particle or lentiviral vector contains an exogenous agent that is capable of targeting a T cell. In some embodiments, the exogenous agent capable of targeting a T cell is a chimeric antigen receptor (CAR), a T cell receptor, an integrin, an ion channel, a pore forming protein, a Toll-Like Receptor, an interleukin receptor, a cell adhesion protein, or a transport protein.

In some embodiments, the CAR is or comprises a first generation CAR comprising an antigen binding domain, a transmembrane domain, and signaling domain (e.g., one, two or three signaling domains). In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an scFv or Fab.

In some embodiments, a CAR antigen binding domain is or comprises an antibody or antigen-binding portion thereof. In some embodiments, a CAR antigen binding domain is or comprises an scFv or Fab. In some embodiments a CAR antigen binding domain comprises an scFv or Fab fragment of a T-cell alpha chain antibody; T-cell β chain antibody; T-cell γ chain antibody; T-cell δ chain antibody; CCR7 antibody; CD3 antibody; CD4 antibody; CD5 antibody; CD7 antibody; CD8 antibody; CD11b antibody; CD11c antibody; CD16 antibody; CD19 antibody; CD20 antibody; CD21 antibody; CD22 antibody; CD25 antibody; CD28 antibody; CD34 antibody; CD35 antibody; CD40 antibody; CD45RA antibody; CD45RO antibody; CD52 antibody; CD56 antibody; CD62L antibody; CD68 antibody; CD80 antibody; CD95 antibody; CD117 antibody; CD127 antibody; CD133 antibody; CD137 (4-1 BB) antibody; CD163 antibody; F4/80 antibody; IL-4Ra antibody; Sca-1 antibody; CTLA-4 antibody; GITR antibody GARP antibody; LAP antibody; granzyme B antibody; LFA-1 antibody; MR1 antibody; uPAR antibody; or transferrin receptor antibody.

In some embodiments, a CAR binding domain binds to a cell surface antigen of a cell. In some embodiments, a cell surface antigen is characteristic of one type of cell. In some embodiments, a cell surface antigen is characteristic of more than one type of cell.

In some embodiments, the antigen binding domain of the CAR targets an antigen characteristic of a T cell. In some embodiments, the antigen characteristic of a T cell is selected from a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, AKT1; AKT2; AKT3; ATF2; BCL10; CALM1; CD3D (CD3δ); CD3E (CD3ε); CD3G (CD3γ); CD4; CD8; CD28; CD45; CD80 (B7-1); CD86 (B7-2); CD247 (CD3ζ); CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38β); MAPK12 (p38γ); MAPK13 (p38δ); MAPK14 (p38a); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

In some embodiments, the antigen binding domain of the CAR targets an antigen characteristic of a disorder. In some embodiments, the disease or disorder is associates with CD4+ T cells. In some embodiments, the disease or disorder is associated with CD8+ T cells.

In some embodiments, the CAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, the transmembrane domain comprises at least a transmembrane region(s) of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof.

In some embodiments, the CAR comprises at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the CAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments the intracellular signaling domain includes intracellular components of a 4-1BB signaling domain and a CD3-zeta signaling domain. In some embodiments, the intracellular signaling domain includes intracellular components of a CD28 signaling domain and a CD3zeta signaling domain.

In some embodiments, the CAR comprises an extracellular antigen binding domain (e.g., antibody or antibody fragment, such as an scFv) that binds to an antigen (e.g. tumor antigen), a spacer (e.g. containing a hinge domain, such as any as described herein), a transmembrane domain (e.g. any as described herein), and an intracellular signaling domain (e.g. any intracellular signaling domain, such as a primary signaling domain or costimulatory signaling domain as described herein). In some embodiments, the intracellular signaling domain is or includes a primary cytoplasmic signaling domain. In some embodiments, the intracellular signaling domain additionally includes an intracellular signaling domain of a costimulatory molecule (e.g., a costimulatory domain). Examples of exemplary components of a CAR are described in Table 6. In provided aspects, the sequences of each component in a CAR can include any combination listed in Table 6.

TABLE 6
CAR components and Exemplary Sequences

| | | |
|---|---|---|
| | | SEQ |
| | | ID |
| Component | Sequence | NO |

Extracellular binding domain

| | | |
|---|---|---|
| Anti-CD19 | DIQMTQTTSSLSASLGDRVTISCRASQDISKY | 419 |
| scFv (FMC63) | LNWYQQKPDGTVKLLIYHTSRLHSGVPSRFS | |
| | GSGSGTDYSLTISNLEQEDIATYFCQQGNTLP | |
| | YTFGGGTKLEITGSTSGSGKPGSGEGSTKGE | |
| | VKLQESGPGLVAPSQSLSVTCTVSGVSLPDY | |
| | GVSWIRQPPRKGLEWLGVIWGSETTYYNSA | |
| | LKSRLTIIKDNSKSQVFLKMNSLQTDDTAIYY | |
| | CAKHYYYGGSYAMDYWGQGTSVTVSS | |
| Anti-CD19 | DIQMTQTTSSLSASLGDRVTISCRASQDISKY | 420 |
| scFv (FMC63) | LNWYQQKPDGTVKLLIYHTSRLHSGVPSRFS | |
| | GSGSGTDYSLTISNLEQEDIATYFCQQGNTLP | |
| | YTFGGGTKLEITGGGGSGGGGSGGGGSEVK | |
| | LQESGPGLVAPSQSLSVTCTVSGVSLPDYGV | |
| | SWIRQPPRKGLEWLGVIWGSETTYYNSALKS | |
| | RLTIIKDNSKSQVFLKMNSLQTDDTAIYYCA | |
| | KHYYYGGSYAMDYWGQGTSVTVSS | |

Spacer (e.g. hinge)

| | | |
|---|---|---|
| IgG4 Hinge | ESKYGPPCPPCP | 421 |
| CD8 Hinge | TTTPAPRPPTPAPTIASQPLSLRPE | 422 |
| CD28 | IEVMYPPPYLDNEKSNGTIIHVKGKHLCPSPL | 423 |
| | FPGPSKP | |

Transmembrane

| | | |
|---|---|---|
| CD8 | ACRPAAGGAVHTRGLDFACDIYIWAPLAGT | 424 |
| | CGVLLLSLVITLYC | |
| CD28 | FWVLVVVGGVLACYSLLVTVAFIIFWV | 425 |
| CD28 | FWVLVVVGGVLACYSLLVTVAFIIFWV | 426 |

Costimulatory domain

| | | |
|---|---|---|
| CD28 | RSKRSRLLHSDYMNMTPRRPGPTRKHYQPY | 427 |
| | APPRDFAAYRS | |
| 4-1BB | KRGRKKLLYIFKQPFMRPVQTTQEEDGCSCR | 428 |
| | FPEEEEGGCEL | |

Primary Signaling Domain

| | | |
|---|---|---|
| CD3zeta | RVKFSRSADAPAYQQGQNQLYNELNLGRRE | 429 |
| | EYDVLDKRRGRDPEMGGKPRRKNPQEGLY | |
| | NELQKDKMAEAYSEIGMKGERRRGKGHDG | |
| | LYQGLSTATKDTYDALHMQALPPR | |
| CD3zeta | RVKFSRSADAPAYKQGQNQLYNELNLGRRE | 430 |
| | EYDVLDKRRGRDPEMGGKPRRKNPQEGLY | |
| | NELQKDKMAEAYSEIGMKGERRRGKGHDG | |
| | LYQGLSTATKDTYDALHMQALPPR | |

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine-serine doublets.

In addition to the CARs described herein, various chimeric antigen receptors and nucleotide sequences encoding the same are known and would be suitable for fusosomal delivery and reprogramming of target cells in vivo and in vitro as described herein. See, e.g., WO2013040557; WO2012079000; WO2016030414; Smith T, et al., Nature Nanotechnology. 2017. (DOI: 10.1038/NNANO.2017.57), the disclosures of which are herein incorporated by reference in their entirety.

In some embodiments a targeted lipid particle comprising a CAR or a nucleic acid encoding a CAR (e.g., a DNA, a gDNA, a cDNA, an RNA, a pre-MRNA, an mRNA, an miRNA, an siRNA, etc.) is delivered to a target cell. In some embodiments the target cell is an effector cell, e.g., a cell of the immune system that expresses one or more Fc receptors and mediates one or more effector functions. In some embodiments, a target cell may include, but may not be limited to, one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell) and may be from any organism including but not limited to humans, mice, rats, rabbits, and monkeys.

E. Methods of Generating Targeted Lipid Particles

Provided herein is a targeted lipid particle comprising a lipid bilayer, a lumen surrounded by the lipid bilayer, a targeted envelope protein, and a fusogen, in which the targeted envelope protein and fusogen are embedded within the lipid bilayer. In some embodiments, the targeted lipid particle can be a viral particle, a virus-like particle, a nanoparticle, a vesicle, an exosome, a dendrimer, a lentivirus, a viral vector, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a lentiviral vector, a viral based particle, a virus like particle (VLP) or a cell derived particle.

I. Virus-Like Particles

Provided herein are targeted lipid particles that are derived from virus, such as viral particles or virus-like particles, including those derived from retroviruses or lentiviruses. In some embodiments, the targeted lipid particle's bilayer of amphipathic lipids is or comprises the viral envelope. In some embodiments, the targeted lipid particle's bilayer of amphipathic lipids is or comprises lipids derived from a producer cell. In some embodiments, the viral envelope may comprise a fusogen, e.g., a fusogen that is endogenous to the virus or a pseudotyped fusogen. In some embodiments, the targeted lipid particle's lumen or cavity comprises a viral nucleic acid, e.g., a retroviral nucleic acid, e.g., a lentiviral nucleic acid. In some embodiments, the viral nucleic acid may be a viral genome. In some embodiments, the targeted lipid particle further comprises one or more viral non-structural proteins, e.g., in its cavity or lumen. In some embodiments, the targeted lipid particles is or comprises a virus-like particle (VLP). In some embodiments, the VLP does not comprise an envelope. In some embodiments, the VLP comprises an envelope.

In some embodiments, the viral particle or virus-like particle, such as retrovirus or retrovirus-like particle, comprises one or more of gag polyprotein, polymerase (e.g., pol), integrase (e.g., a functional or non-functional variant), protease, and a fusogen. In some embodiments, the targeted lipid particle further comprises rev. In some embodiments, one or more of the aforesaid proteins are encoded in the retroviral genome, and in some embodiments, one or more of the aforesaid proteins are provided in trans, e.g., by a helper cell, helper virus, or helper plasmid. In some embodiments, the targeted lipid particle nucleic acid (e.g., retroviral nucleic acid) comprises one or more of the following nucleic acid sequences: 5′ LTR (e.g., comprising U5 and lacking a functional U3 domain), Psi packaging element (Psi), Central polypurine tract (cPPT) Promoter operatively linked to the payload gene, payload gene (optionally comprising an intron before the open reading frame), Poly A tail sequence, WPRE, and 3′ LTR (e.g., comprising U5 and lacking a functional U3). In some embodiments the targeted lipid particle nucleic acid further comprises one or more insulator element. In some embodiments, the recognition sites are situated between the poly A tail sequence and the WPRE.

In some embodiments, the targeted lipid particle comprises supramolecular complexes formed by viral proteins that self-assemble into capsids. In some embodiments, the targeted lipid particle is a viral particle or virus-like particle derived from viral capsids. In some embodiments, the targeted lipid particle is a viral particle or virus-like particle derived from viral nucleocapsids. In some embodiments, the targeted lipid particle comprises nucleocapsid-derived that retain the property of packaging nucleic acids. In some embodiments, the viral particles or virus-like particles comprises only viral structural glycoproteins. In some embodiments, the targeted lipid particle does not contain a viral genome.

In some embodiments, the targeted lipid particle packages nucleic acids from host cells during the expression process. In some embodiments, the nucleic acids do not encode any genes involved in virus replication. In particular embodiments, the targeted lipid particle is a virus-like particle, e.g. retrovirus-like particle such as a lentivirus-like particle, that is replication defective.

In some cases, the targeted lipid particle is a viral particle that is morphologically indistinguishable from the wild type infectious virus. In some embodiments, the viral particle presents the entire viral proteome as an antigen. In some embodiments, the viral particle presents only a portion of the proteome as an antigen.

In some embodiments, the viral particle or virus-like particle is produced utilizing proteins (e.g., envelope proteins) from a virus within the Paramyxoviridae family In some embodiments, the Paramyxoviridae family comprises members within the Henipavirus genus. In some embodiments, the Henipavirus is or comprises a Hendra (HeV) or a Nipah (NiV) virus. In particular embodiments, the viral particles or virus-like particles incorporate a targeted envelope protein and fusogen as described in Section I.A. and 1.B.

In some embodiments, viral particles or virus-like particles may be produced in multiple cell culture systems including bacteria, mammalian cell lines, insect cell lines, yeast and plant cells.

In some embodiments, the assembly of a viral particle or virus-like particle is initiated by binding of the core protein to a unique encapsidation sequence within the viral genome (e.g. UTR with stem-loop structure). In some embodiments, the interaction of the core with the encapsidation sequence facilitates oligomerization.

In some embodiments, the targeted lipid particle is a virus-like particle which comprises a sequence that is devoid of or lacking viral RNA may be the result of removing or eliminating the viral RNA from the sequence. In some embodiments, this may be achieved by using an endogenous packaging signal binding site on gag. In some embodiments, the endogenous packaging signal binding site is on pol. In some embodiments, the RNA which is to be delivered will contain a cognate packaging signal. In some embodiments, a heterologous binding domain (which is heterologous to gag) located on the RNA to be delivered, and a cognate binding site located on gag or pol, can be used to ensure packaging of the RNA to be delivered. In some embodiments, the heterologous sequence could be non-viral or it could be viral, in which case it may be derived from a different virus. In some embodiments, the vector particles could be used to deliver therapeutic RNA, in which case functional integrase and/or reverse transcriptase is not required. In some embodiments, the vector particles could also be used to deliver a therapeutic gene of interest, in which case pol is typically included.

a. Transfer Vectors

In some embodiments, the retroviral nucleic acid comprises one or more of (e.g., all of): a 5′ promoter (e.g., to control expression of the entire packaged RNA), a 5′ LTR (e.g., that includes R (polyadenylation tail signal) and/or U5 which includes a primer activation signal), a primer binding site, a psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the retroviral nucleic acid further comprises one or more of a cPPT, a WPRE, and/or an insulator element.

A retrovirus typically replicates by reverse transcription of its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV), and lentivirus.

In some embodiments the retrovirus is a Gammaretrovirus. In some embodiments the retrovirus is an Epsilonretrovirus. In some embodiments the retrovirus is an Alpharetrovirus. In some embodiments the retrovirus is a Betaretrovirus. In some embodiments the retrovirus is a Deltaretrovirus. In some embodiments the retrovirus is a Lentivirus. In some embodiments the retrovirus is a Spumaretrovirus. In some embodiments the retrovirus is an endogenous retrovirus.

Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used.

In some embodiments, a vector herein is a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

In some embodiments, a viral vector comprises a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). In some embodiments, a viral vector comprises e.g., a virus or viral particle capable of transferring a nucleic acid into a cell, or to the transferred nucleic acid (e.g., as naked DNA). In some embodiments, a viral vectors and transfer plasmids comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some embodiments, in the vectors described herein at least part of one or more protein coding regions that contribute to or are essential for replication may be absent compared to the corresponding wild-type virus. In some embodiments, the viral vector replication-defective. In some embodiments, the vector is capable of transducing a target non-dividing host cell and/or integrating its genome into a host genome.

In some embodiments, the structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). In some embodiments, the LTRs are involved in proviral integration and transcription. In some embodiments, LTRs serve as enhancer-promoter sequences and can control the expression of the viral genes. In some embodiments, encapsidation of the retroviral RNAs occurs by virtue of a psi sequence located at the 5′ end of the viral genome.

In some embodiments, LTRs are similar sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In some embodiments, for the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. In some embodiments, retroviruses comprise any one or more of the following genes that code for proteins that are involved in the regulation of gene expression: tat, rev, tax and rex.

In some embodiments, the structural genes gag, pol and env, gag encodes the internal structural protein of the virus. In some embodiments, Gag protein is proteolytically processed into the mature proteins MA (matrix), CA (capsid) and NC (nucleocapsid). In some embodiments, the pol gene encodes the reverse transcriptase (RT), which contains DNA polymerase, associated RNase H and integrase (IN), which mediate replication of the genome. In some embodiments, the env gene encodes the surface (SU) glycoprotein and the transmembrane (TM) protein of the virion, which form a complex that interacts specifically with cellular receptor proteins. In some embodiments, the interaction promotes infection by fusion of the viral membrane with the cell membrane.

In some embodiments, a replication-defective retroviral vector genome gag, pol and env may be absent or not functional. In some embodiments, the R regions at both ends of the RNA are typically repeated sequences. In some embodiments, U5 and U3 represent unique sequences at the 5′ and 3′ ends of the RNA genome respectively.

In some embodiments, retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. In some embodiments, proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11).

In some embodiments, in addition to protease, reverse transcriptase and integrase, non-primate lentiviruses contain a fourth pol gene product which codes for a dUTPase. In some embodiments, this a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. In some embodiments, infection of the target cell can comprise reverse transcription and integration into the target cell genome. In some embodiments, the RLV typically carries non-viral coding sequences which are to be delivered by the vector to the target cell. In some embodiments, an RLV is incapable of independent replication to produce infectious retroviral particles within the target cell. In some embodiments, the RLV lacks a functional gag-pol and/or env gene and/or other genes involved in replication. In some embodiments, the vector may be configured as a split-intron vector, e.g., as described in PCT patent application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

In some embodiments, a minimal lentiviral genome may comprise, e.g., (5′)R-U5-one or more first nucleotide sequences-U3-R(3′). In some embodiments, the plasmid vector used to produce the lentiviral genome within a source cell can also include transcriptional regulatory control sequences operably linked to the lentiviral genome to direct transcription of the genome in a source cell. In some embodiments, the regulatory sequences may comprise the natural sequences associated with the transcribed retroviral sequence, e.g., the 5′ U3 region, or they may comprise a heterologous promoter such as another viral promoter, for example the CMV promoter. In some embodiments, lentiviral genomes comprise additional sequences to promote efficient virus production. In some embodiments, in the case of HIV, rev and RRE sequences may be included. In some embodiments, alternatively or combination, codon optimization may be used, e.g., the gene encoding the exogenous agent may be codon optimized, e.g., as described in WO 01/79518, which is herein incorporated by reference in its entirety. In some embodiments, alternative sequences which perform a similar or the same function as the rev/RRE system may also be used. In some embodiments, a functional analogue of the rev/RRE system is found in the Mason Pfizer monkey virus. In some embodiments, this is known as CTE and comprises an RRE-type sequence in the genome which is believed to interact with a factor in the infected cell. The cellular factor can be thought of as a rev analogue. In some embodiments, CTE may be used as an alternative to the rev/RRE system. In some embodiments, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non-dividing cells.

In some embodiments, the deletion of additional genes may permit vectors to be produced without the genes associated with disease in lentiviral (e.g. HIV) infections. In some embodiments, tat is associated with disease. In some embodiments, the deletion of additional genes permits the vector to package more heterologous DNA. In some embodiments, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. In some embodiments, the concentration of free dNTPs in the cytoplasm increases as Vpx degrades SAMHD1 and reverse transcription activity is increased, thus facilitating reverse transcription of the retroviral genome and integration into the target cell genome.

In some embodiments, different cells differ in their usage of particular codons. In some embodiments, this codon bias corresponds to a bias in the relative abundance of particular tRNAs in the cell type. In some embodiments, by altering the codons in the sequence so that they are tailored to match with the relative abundance of corresponding tRNAs, it is possible to increase expression. In some embodiments, it is possible to decrease expression by deliberately choosing codons for which the corresponding tRNAs are known to be rare in the particular cell type. In some embodiments, an additional degree of translational control is available. An additional description of codon optimization is found, e.g., in WO 99/41397, which is herein incorporated by reference in its entirety.

In some embodiments viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

In some embodiments, the strategy for codon optimized gag-pol sequences can be used in relation to any retrovirus, e.g., EIAV, FIV, BIV, CAEV, VMR, SIV, HIV-I and HIV-2. In addition this method could be used to increase expression of genes from HTLV-I, HTLV-2, HFV, HSRV and human endogenous retroviruses (HERV), MLV and other retroviruses.

In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, the lentiviral vector is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a letivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798):1316-1332). In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

In some embodiments, most or all of the viral vector backbone sequences are derived from a lentivirus, e.g., HIV-1. However, it is to be understood that many different sources of retroviral and/or lentiviral sequences can be used or combined and numerous substitutions and alterations in certain of the lentiviral sequences may be accommodated without impairing the ability of a transfer vector to perform the functions described herein. A variety of lentiviral vectors are described in Naldini et al., (1996a, 1996b, and 1998); Zufferey et al., (1997); Dull et al., 1998, U.S. Pat. Nos. 6,013,516; and 5,994,136, many of which may be adapted to produce a retroviral nucleic acid.

In some embodiments, at each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi NI sequence) for encapsidation of the viral genome.

In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. This is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly(A) sequence The 3′ LTR, the 5′ LTR, or both 3′ and 5′ LTRs, may be modified LTRs.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

In some embodiments, the R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

In some embodiments, the retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-1.

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE.

In some embodiments, a retroviral nucleic acid described herein lacks or does not comprise a posttranscriptional regulatory element such as a WPRE or HPRE.

In some embodiments, elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATTAAA, AGTAAA, a bovine growth hormone polyA sequence (BGHpA), a rabbit β-globin polyA sequence (rβgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase exogenous gene expression (e.g., poly (A) sequences), and may optionally comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

b. Packaging Vectors and Producer Cells

Large scale viral particle production is often useful to achieve a desired viral titer. Viral particles can be produced by transfecting a transfer vector into a packaging cell line that comprises viral structural and/or accessory genes, e.g., gag, pol, env, tat, rev, vif, vpr, vpu, vpx, or nef genes or other retroviral genes.

In some embodiments, the packaging vector is an expression vector or viral vector that lacks a packaging signal and comprises a polynucleotide encoding one, two, three, four or more viral structural and/or accessory genes. Typically, the packaging vectors are included in a producer cell, and are introduced into the cell via transfection, transduction or infection. A retroviral, e.g., lentiviral, transfer vector can be introduced into a producer cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

In some embodiments, producer cell lines include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRCS cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In embodiments, the packaging cells are 293 cells, 293T cells, or A549 cells.

In some embodiments, a source cell line includes a cell line which is capable of producing recombinant retroviral particles, comprising a producer cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the producer cells, e.g., by cell lysis, or collection of the supernatant of the cell culture. Optionally, the collected virus particles may be enriched or purified.

In some embodiments, the source cell comprises one or more plasmids coding for viral structural proteins and replication enzymes (e.g., gag, pol and env) which can package viral particles. In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the exogenous agent (e.g., a retroviral nucleic acid encoding the exogenous agent) is also integrated into the source cell genome.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid.

2 Cell-Derived Particles

Provided herein are targeted lipid particles that comprise a naturally derived membrane. In some embodiments, the naturally derived membrane comprises membrane vesicles prepared from cells or tissues. In some embodiments, the targeted lipid particle comprises a vesicle that is obtainable from a cell. In some embodiments, the targeted lipid particle comprises a microvesicle, an exosome, a membrane enclosed body, an apoptotic body (from apoptotic cells), a particle (which may be derived from e.g. platelets), an ectosome (derivable from, e.g., neutrophiles and monocytes in serum), a prostatosome (obtainable from prostate cancer cells), or a cardiosome (derivable from cardiac cells).

In some embodiments, the source cell is an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial gial cell, a bone marrow stromal cell, a pancreatic progenitor cell, an endothelial progenitor cell, a blast cell), or an immortalized cell (e.g., HeEa, HEK293, MRC-5, WI-38, IMR 90, IMR 91, PER.C6, HT-1080, or BJ cell). In some embodiments, the source cell is other than a 293 cell, HEK cell, human endothelial cell, or a human epithelial cell, monocyte, macrophage, dendritic cell, or stem cell.

In some embodiments, the targeted lipid particle has a density of <1, 1-1.1, 1.05-1.15, 1.1-1.2, 1.15-1.25, 1.2-1.3, 1.25-1.35, or >1.35 g/ml. In some embodiments, the targeted lipid particle composition comprises less than 0.01%, 0.05%, 0.1%, 0.5%, 1%, 1.5%, 2%, 2.5%, 3%, 4%, 5%, or 10% source cells by protein mass or less than 0.01%, 0.05%, 0.1%, 0.5%, 1%, 1.5%, 2%, 2.5%, 3%, 4%, 5%, or 10% of cells having a functional nucleus.

In embodiments, the targeted lipid particle has a size, or the population of targeted lipid particles have an average size, that is less than about 0.01%, 0.05%, 0.1%, 0.5%, 1%, 2%, 3%, 4%, 5%, 10%, 20%, 30%, 40%, 50%, 60%, 70%, 80%, 90%, of that of the source cell.

In some embodiments the targeted lipid particle comprises an extracellular vesicle, e.g., a cell-derived vesicle comprising a membrane that encloses an internal space and has a smaller diameter than the cell from which it is derived. In embodiments the extracellular vesicle has a diameter from 20 nm to 1000 nm. In embodiments the targeted lipid particle comprises an apoptotic body, a fragment of a cell, a vesicle derived from a cell by direct or indirect manipulation, a vesiculated organelle, and a vesicle produced by a living cell (e.g., by direct plasma membrane budding or fusion of the late endosome with the plasma membrane). In embodiments the extracellular vesicle is derived from a living or dead organism, explanted tissues or organs, or cultured cells.

In embodiments, the targeted lipid particle comprises a nanovesicle, e.g., a cell-derived small (e.g., between 20-250 nm in diameter, or 30-150 nm in diameter) vesicle comprising a membrane that encloses an internal space, and which is generated from said cell by direct or indirect manipulation. The production of nanovesicles can, in some instances, result in the destruction of the source cell. The nanovesicle may comprise a lipid or fatty acid and polypeptide.

In embodiments, the targeted lipid particle comprises an exosome. In embodiments, the exosome is a cell-derived small (e.g., between 20-300 nm in diameter, or 40-200 nm in diameter) vesicle comprising a membrane that encloses an internal space, and which is generated from said cell by direct plasma membrane budding or by fusion of the late endosome with the plasma membrane. In embodiments, production of exosomes does not result in the destruction of the source cell. In embodiments, the exosome comprises lipid or fatty acid and polypeptide. Exemplary exosomes and other membrane-enclosed bodies are also described in WO/2017/161010, WO/2016/077639, US20160168572, US20150290343, and US20070298118, each of which is incorporated by reference herein in its entirety.

In some embodiments, the targeted lipid particle is derived from a source cell with a genetic modification which results in increased expression of an immunomodulatory agent. In some embodiments, the immunosuppressive agent is on an exterior surface of the cell. In some embodiments, the immunosuppressive agent is incorporated into the exterior surface of the targeted lipid particle. In some embodiments, the targeted lipid particle comprises an immunomodulatory agent attached to the surface of the solid particle by a covalent or non-covalent bond.

c. A. Generation of Cell-Derived Particles

In some embodiments, targeted lipid particles are generated by inducing budding of an exosome, microvesicle, membrane vesicle, extracellular membrane vesicle, plasma membrane vesicle, giant plasma membrane vesicle, apoptotic body, mitoparticle, pyrenocyte, lysosome, or other membrane enclosed vesicle.

In some embodiments, targeted lipid particles are generated by inducing cell enucleation. Enucleation may be performed using assays such as genetic, chemical (e.g., using Actinomycin D, see Bayona-Bafaluyet al., “A chemical enucleation method for the transfer of mitochondrial DNA to p° cells” Nucleic Acids Res. 2003 Aug. 15; 31(16): e98), mechanical methods (e.g., squeezing or aspiration, see Lee et al., “A comparative study on the efficiency of two enucleation methods in pig somatic cell nuclear transfer: effects of the squeezing and the aspiration methods.” Anim Biotechnol. 2008; 19(2):71-9), or combinations thereof.

In some embodiments, the targeted lipid particles are generated by inducing cell fragmentation. In some embodiments, cell fragmentation can be performed using the following methods, including, but not limited to: chemical methods, mechanical methods (e.g., centrifugation (e.g., ultracentrifugation, or density centrifugation), freeze-thaw, or sonication), or combinations thereof.

In some embodiments, the targeted lipid particle is a microvesicle. In some embodiments the microvesicle has a diameter of about 100 nm to about 2000 nm. In some embodiments, a targeted lipid particle comprises a cell ghost. In some embodiments, a vesicle is a plasma membrane vesicle, e.g. a giant plasma membrane vesicle.

In some embodiments, the source cell used to make the targeted lipid particle will not be available for testing after the targeted lipid particle is made.

In some embodiments, a characteristic of a targeted lipid particle is described by comparison to a reference cell. In embodiments, the reference cell is the source cell. In embodiments, the reference cell is a HeLa, HEK293, HFF-1, MRC-5, WI-38, IMR 90, IMR 91, PER.C6, HT-1080, or BJ cell. In some embodiments, a characteristic of a population of targeted lipid particle is described by comparison to a population of reference cells, e.g., a population of source cells, or a population of HeLa, HEK293, MRC-5, WI-38, IMR 90, IMR 91, PER.C6, HT-1080, or BJ cells.

### III. PHARMACEUTICAL COMPOSITIONS

The present disclosure also provides, in some aspects, a pharmaceutical composition comprising the targeted lipid particle composition described herein and pharmaceutically acceptable carrier. The pharmaceutical compositions can include any of the described targeted lipid particles.

In some embodiments, the targeted lipid particle meets a pharmaceutical or good manufacturing practices (GMP) standard. In some embodiments, the targeted lipid particle was made according to good manufacturing practices (GMP). In some embodiments, the targeted lipid particle has a pathogen level below a predetermined reference value, e.g., is substantially free of pathogens. In some embodiments, the targeted lipid particle has a contaminant level below a predetermined reference value, e.g., is substantially free of contaminants In some embodiments, the targeted lipid particle has low immunogenicity.

In some embodiments, provided herein are the use of pharmaceutical compositions of the invention or salts thereof to practice the methods of the invention. Such a pharmaceutical composition may consist of at least one compound or conjugate of the invention or a salt thereof in a form suitable for administration to a subject, or the pharmaceutical composition may comprise at least one compound or conjugate of the invention or a salt thereof, and one or more pharmaceutically acceptable carriers, one or more additional ingredients, or some combination of these. In some embodiments, the compound or conjugate of the invention may be present in the pharmaceutical composition in the form of a physiologically acceptable salt, such as in combination with a physiologically acceptable cation or anion, as is well known in the art.

In some embodiments, the pharmaceutical compositions useful for practicing the methods of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

In some embodiments, the relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. In some embodiments, the composition may comprise between 0.1% and 100% (w/w) active ingredient.

In some embodiments, pharmaceutical compositions that are useful in the methods of the invention may be suitably developed for oral, rectal, vaginal, parenteral, topical, pulmonary, intranasal, buccal, ophthalmic, or another route of administration. In some embodiments, a composition useful within the methods of the invention may be directly administered to the skin, vagina or any other tissue of a mammal. In some embodiments, formulations include liposomal preparations, resealed erythrocytes containing the active ingredient, and immunologically based formulations. In some embodiments, the route(s) of administration will be readily apparent to the skilled artisan and will depend upon any number of factors including the type and severity of the disease being treated, the type and age of the veterinary or human subject being treated, and the like.

In some embodiments, formulations of the pharmaceutical compositions described herein may be prepared by any method known or hereafter developed in the art of pharmacology. In some embodiments, preparatory methods include the step of bringing the active ingredient into association with a carrier or one or more other accessory ingredients, and then, if necessary or desirable, shaping or packaging the product into a desired single- or multi-dose unit.

In some embodiments, a “unit dose” is a discrete amount of the pharmaceutical composition comprising a predetermined amount of the active ingredient. In some embodiments, the amount of the active ingredient is generally equal to the dosage of the active ingredient that would be administered to a subject or a convenient fraction of such a dosage such as, for example, one-half or one-third of such a dosage. In some embodiments, the unit dosage form may be for a single daily dose or one of multiple daily doses (e.g., about 1 to 4 or more times per day). In some embodiments, when multiple daily doses are used, the unit dosage form may be the same or different for each dose.

In some embodiments, although the descriptions of pharmaceutical compositions provided herein are principally directed to pharmaceutical compositions that are suitable for ethical administration to humans, it will be understood by the skilled artisan that such compositions are generally suitable for administration to animals of all sorts. In some embodiments, modification of pharmaceutical compositions suitable for administration to humans in order to render the compositions suitable for administration to various animals is well understood, and the ordinarily skilled veterinary pharmacologist may design and perform such modification with merely ordinary, if any, experimentation. In some embodiments, subjects to which administration of the pharmaceutical compositions of the invention is contemplated include humans and other primates, mammals including commercially relevant mammals such as cattle, pigs, horses, sheep, cats, and dogs.

In some of any embodiments, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. In some embodiments, pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

In some embodiments, the carrier may be a solvent or dispersion medium containing, for example, water, ethanol, polyol (for example, glycerol, propylene glycol, and liquid polyethylene glycol, and the like), suitable mixtures thereof, and vegetable oils. In some embodiments, the proper fluidity may be maintained, for example, by the use of a coating such as lecithin, by the maintenance of the required particle size in the case of dispersion and by the use of surfactants. In some embodiments, prevention of the action of microorganisms may be achieved by various antibacterial and antifungal agents, for example, parabens, chlorobutanol, phenol, ascorbic acid, thimerosal, and the like. In some embodiments, it is preferable to include isotonic agents, for example, sugars, sodium chloride, or polyalcohols such as mannitol and sorbitol, in the composition. In some embodiments, prolonged absorption of the injectable compositions may be brought about by including in the composition an agent that delays absorption, for example, aluminum monostearate or gelatin. In one embodiment, the pharmaceutically acceptable carrier is not DMSO alone.

In some embodiments, formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. In some embodiments, the pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. In some embodiments, pharmaceutical preparations may also be combined where desired with other active agents, e.g., other analgesic agents.

In some embodiments, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. In some embodiments, “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

In some embodiments, the composition of the invention may comprise a preservative from about 0.005% to 2.0% by total weight of the composition. In some embodiments, the preservative is used to prevent spoilage in the case of exposure to contaminants in the environment. In some embodiments, examples of preservatives useful in accordance with the invention included but are not limited to those selected from the group consisting of benzyl alcohol, sorbic acid, parabens, imidurea and combinations thereof. In some embodiments, a particularly preferred preservative is a combination of about 0.5% to 2.0% benzyl alcohol and 0.05% to 0.5% sorbic acid.

In some embodiments, the composition preferably includes an anti-oxidant and a chelating agent that inhibits the degradation of the compound. In some embodiments, antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. In some embodiments, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. In some embodiments, the chelating agent is useful for chelating metal ions in the composition that may be detrimental to the shelf life of the formulation. In some embodiments, other suitable and equivalent antioxidants and chelating agents may be substituted therefore as would be known to those skilled in the art.

In some embodiments, liquid suspensions may be prepared using conventional methods to achieve suspension of the active ingredient in an aqueous or oily vehicle. In some embodiments, aqueous vehicles include, for example, water, and isotonic saline. In some embodiments, oily vehicles include, for example, almond oil, oily esters, ethyl alcohol, vegetable oils such as arachis, olive, sesame, or coconut oil, fractionated vegetable oils, and mineral oils such as liquid paraffin. In some embodiments, liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. In some embodiments, oily suspensions may further comprise a thickening agent. In some embodiments, suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. In some embodiments, dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

In some embodiments, liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. In some embodiments, liquid solutions of the pharmaceutical composition of the invention may comprise each of the components described with regard to liquid suspensions, it being understood that suspending agents will not necessarily aid dissolution of the active ingredient in the solvent. In some embodiments, aqueous solvents include, for example, water, and isotonic saline. In some embodiments, oily solvents include, for example, almond oil, oily esters, ethyl alcohol, vegetable oils such as arachis, olive, sesame, or coconut oil, fractionated vegetable oils, and mineral oils such as liquid paraffin.

In some embodiments, powdered and granular formulations of a pharmaceutical preparation of the invention may be prepared using known methods. In some embodiments, formulations may be administered directly to a subject, used, for example, to form tablets, to fill capsules, or to prepare an aqueous or oily suspension or solution by addition of an aqueous or oily vehicle thereto. In some of any embodiments, formulations may further comprise one or more of dispersing or wetting agent, a suspending agent, and a preservative. Additional excipients, such as fillers and sweetening, flavoring, or coloring agents, may also be included in these formulations.

In some embodiments, a pharmaceutical composition of the invention may also be prepared, packaged, or sold in the form of oil-in-water emulsion or a water-in-oil emulsion. In some embodiments, the oily phase may be a vegetable oil such as olive or arachis oil, a mineral oil such as liquid paraffin, or a combination of these. In some embodiments, compositions further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. In some embodiments, emulsions may also contain additional ingredients including, for example, sweetening or flavoring agents.

### IV. METHODS OF TREATMENT

In some embodiments, the targeted lipid particles provided herein, or pharmaceutical compositions thereof as described herein can be administered to a subject, e.g. a mammal, e.g. a human. In such embodiments, the subject may be at risk of, may have a symptom of, or may be diagnosed with or identified as having, a particular disease or condition. In one embodiment, the subject has cancer. In one embodiment, the subject has an infectious disease. In some embodiments, the targeted lipid particle contains nucleic acid sequences encoding an exogenous agent for treating the disease or condition in the subject. For example, the exogenous agent is one that targets or is specific for a protein of a neoplastic cells and the targeted lipid particle is administered to a subject for treating a tumor or cancer in the subject. In another example, the exogenous agent is an inflammatory mediator or immune molecule, such as a cytokine, and targeted lipid particle is administered to a subject for treating any condition in which it is desired to modulate (e.g. increase) the immune response, such as a cancer or infectious disease. In some embodiments, the targeted lipid particle is administered in an effective amount or dose to effect treatment of the disease, condition or disorder. Provided herein are uses of any of the provided targeted lipid particles in such methods and treatments, and in the preparation of a medicament in order to carry out such therapeutic methods. In some embodiments, the methods are carried out by administering the targeted lipid particle or compositions comprising the same, to the subject having, having had, or suspected of having the disease or condition or disorder. In some embodiments, the methods thereby treat the disease or condition or disorder in the subject. Also provided herein are uses of any of the compositions, such as pharmaceutical compositions provided herein, for the treatment of a disease, condition or disorder associated with a particular gene or protein targeted by or provided by the exogenous agent.

In some embodiments, the provided methods or uses involve administration of a pharmaceutical composition comprising oral, inhaled, transdermal or parenteral (including intravenous, intratumoral, intraperitoneal, intramuscular, intracavity, and subcutaneous) administration. In some embodiments, the targeted lipid particle may be administered alone or formulated as a pharmaceutical composition. In some embodiments, the targeted lipid particle or compositions described herein can be administered to a subject, e.g., a mammal, e.g., a human. In some of any embodiments, the subject may be at risk of, may have a symptom of, or may be diagnosed with or identified as having, a particular disease or condition (e.g., a disease or condition described herein). In some embodiments, the disease is a disease or disorder.

In some embodiments, the targeted lipid particles may be administered in the form of a unit-dose composition, such as a unit dose oral, parenteral, transdermal or inhaled composition. In some embodiments, the compositions are prepared by admixture and are adapted for oral, inhaled, transdermal or parenteral administration, and as such may be in the form of tablets, capsules, oral liquid preparations, powders, granules, lozenges, reconstitutable powders, injectable and infusable solutions or suspensions or suppositories or aerosols.

In some embodiments, the regimen of administration may affect what constitutes an effective amount. In some embodiments, the therapeutic formulations may be administered to the subject either prior to or after a diagnosis of disease. In some embodiments, several divided dosages, as well as staggered dosages may be administered daily or sequentially, or the dose may be continuously infused, or may be a bolus injection. In some embodiments, the dosages of the therapeutic formulations may be proportionally increased or decreased as indicated by the exigencies of the therapeutic or prophylactic situation.

In some embodiments, the administration of the compositions of the present invention to a subject, preferably a mammal, more preferably a human, may be carried out using known procedures, at dosages and for periods of time effective to prevent or treat disease. In some embodiments, an effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. In some embodiments, the dosage regimens may be adjusted to provide the optimum therapeutic response. In some embodiments, several divided doses may be administered daily or the dose may be proportionally reduced as indicated by the exigencies of the therapeutic situation. In some embodiments, the effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

In some embodiments, the compound may be administered to a subject as frequently as several times daily, or it may be administered less frequently, such as once a day, once a week, once every two weeks, once a month, or even less frequently, such as once every several months or even once a year or less. In some embodiments, the amount of compound dosed per day may be administered, in non-limiting examples, every day, every other day, every 2 days, every 3 days, every 4 days, or every 5 days. In some embodiments, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

In some embodiments, dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. In some embodiments, the physician or veterinarian could start doses of the compounds of the invention employed in the pharmaceutical composition at levels lower than that required in order to achieve the desired therapeutic effect and gradually increase the dosage until the desired effect is achieved.

In some embodiments, it is especially advantageous to formulate the compound in dosage unit form for ease of administration and uniformity of dosage. In some embodiments, dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. In some embodiments, the dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In some embodiments, the term “container” includes any receptacle for holding the pharmaceutical composition. In some embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. It should be understood that the instructions for use of the pharmaceutical composition may be contained on the packaging containing the pharmaceutical composition, and as such the instructions form an increased functional relationship to the packaged product. In some embodiments, instructions may contain information pertaining to the compound's ability to perform its intended function, e.g., treating or preventing a disease in a subject, or delivering an imaging or diagnostic agent to a subject.

In some embodiments, routes of administration of any of the compositions disclosed herein include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

In some of any embodiments, suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like.

In some embodiments, the targeted lipid particle composition comprising an exogenous agent or cargo, may be used to deliver such exogenous agent or cargo to a cell tissue or subject. In some embodiments, delivery of a cargo by administration of a targeted lipid particle composition described herein may modify cellular protein expression levels. In certain embodiments, the administered composition directs upregulation of (via expression in the cell, delivery in the cell, or induction within the cell) of one or more cargo (e.g., a polypeptide or mRNA) that provide a functional activity which is substantially absent or reduced in the cell in which the polypeptide is delivered. In some embodiments, the missing functional activity may be enzymatic, structural, or regulatory in nature. In some embodiments, the administered composition directs up-regulation of one or more polypeptides that increases (e.g., synergistically) a functional activity which is present but substantially deficient in the cell in which the polypeptide is upregulated. In some of any embodiments, the administered composition directs downregulation of (via expression in the cell, delivery in the cell, or induction within the cell) of one or more cargo (e.g., a polypeptide, siRNA, or miRNA) that repress a functional activity which is present or upregulated in the cell in which the polypeptide, siRNA, or miRNA is delivered. In some of any embodiments, the upregulated functional activity may be enzymatic, structural, or regulatory in nature. In some embodiments, the administered composition directs down-regulation of one or more polypeptides that decreases (e.g., synergistically) a functional activity which is present or upregulated in the cell in which the polypeptide is downregulated. In some embodiments, the administered composition directs upregulation of certain functional activities and downregulation of other functional activities.

In some of any embodiments, the targeted lipid particle composition (e.g., one comprising mitochondria or DNA) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the targeted lipid particle composition comprises an exogenous protein), the effect lasts for less than 1, 2, 3, 4, 5, 6, or 7 days, 2, 3, or 4 weeks, or 1, 2, 3, 6, or 12 months.

In some of any embodiments, the targeted lipid particle composition described herein is delivered ex-vivo to a cell or tissue, e.g., a human cell or tissue. In embodiments, the composition improves function of a cell or tissue ex-vivo, e.g., improves cell viability, respiration, or other function (e.g., another function described herein).

In some embodiments, the composition is delivered to an ex vivo tissue that is in an injured state (e.g., from trauma, disease, hypoxia, ischemia or other damage).

In some embodiments, the composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the composition is delivered to the tissue or organ before, during and/or after transplantation.

In some embodiments, the composition is delivered, administered or contacted with a cell, e.g., a cell preparation. In some embodiments, the cell preparation may be a cell therapy preparation (a cell preparation intended for administration to a human subject). In embodiments, the cell preparation comprises cells expressing a chimeric antigen receptor (CAR), e.g., expressing a recombinant CAR. The cells expressing the CAR may be, e.g., T cells, Natural Killer (NK) cells, cytotoxic T lymphocytes (CTL), regulatory T cells. In embodiments, the cell preparation is a neural stem cell preparation. In embodiments, the cell preparation is a mesenchymal stem cell (MSC) preparation. In embodiments, the cell preparation is a hematopoietic stem cell (HSC) preparation. In embodiments, the cell preparation is an islet cell preparation.

In some embodiments, the targeted lipid particle compositions described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein).

In some embodiments, the source of targeted lipid particles are from the same subject that is administered a targeted lipid particle composition. In other embodiments, they are different. In some embodiments, the source of targeted lipid particles and recipient tissue may be autologous (from the same subject) or heterologous (from different subjects). In some embodiments, the donor tissue for targeted lipid particle compositions described herein may be a different tissue type than the recipient tissue. In some embodiments, the donor tissue may be muscular tissue and the recipient tissue may be connective tissue (e.g., adipose tissue). In other embodiments, the donor tissue and recipient tissue may be of the same or different type, but from different organ systems.

In some embodiments, the targeted lipid particle composition described herein may be administered to a subject having a cancer, an autoimmune disease, an infectious disease, a metabolic disease, a neurodegenerative disease, or a genetic disease (e.g., enzyme deficiency). In some embodiments, the subject is in need of regeneration.

In some embodiments, the targeted lipid particle is co-administered with an inhibitor of a protein that inhibits membrane fusion. For example, Suppressyn is a human protein that inhibits cell-cell fusion (Sugimoto et al., “A novel human endogenous retroviral protein inhibits cell-cell fusion” Scientific Reports 3: 1462 (DOI: 10.1038/srep01462)). In some embodiments, the targeted lipid particle particles is co-administered with an inhibitor of sypressyn, e.g., a siRNA or inhibitory antibody.

### V. EXEMPLARY EMBODIMENTS

Among the provided embodiments are:

1. A targeted lipid particle, comprising:

(a) a lipid bilayer enclosing a lumen,

(b) a henipavirus F protein molecule or biologically active portion thereof; and

(c) a targeted envelope protein comprising (i) a henipavirus envelope attachment glycoprotein G (G protein) or a biologically active portion thereof and (ii) single domain antibody (sdAb) variable domain, wherein the sdAb variable domain is attached to the C-terminus of the G protein or the biologically active portion thereof, wherein the F protein molecule or the biologically active portion thereof and the targeted envelope protein are embedded in the lipid bilayer.

2. The targeted lipid particle of embodiment 1, wherein the single domain antibody is attached to the G protein via a linker.

3. The targeted lipid particle of embodiment 2, wherein the linker is a peptide linker.

4. A targeted lipid particle, comprising:

(a) a lipid bilayer enclosing a lumen,

(b) a henipavirus F protein molecule or biologically active portion thereof; and

(c) a targeted envelope protein comprising a henipavirus envelope attachment glycoprotein G (G protein) or biologically active portion thereof attached to a single domain antibody (sdAb) variable domain via a peptide linker, wherein the single domain antibody binds to a cell surface molecule of a target cell,

wherein the F protein molecule or biologically active portion thereof and the targeted envelope protein are embedded in the lipid bilayer.

5. The targeted lipid particle of any of embodiments 1-4, wherein N-terminus of the F protein molecule or biologically active portion thereof is exposed on the outside of lipid bilayer.

6. The targeted lipid particle of any of embodiments 1-5, wherein the C-terminus of the G protein is exposed on the outside of the lipid bilayer.

7. The targeted lipid particle of any of embodiments 1-6, wherein the single domain antibody binds a cell surface molecule present on a target cell.

8. The targeted lipid particle of embodiment 7, wherein the cell surface molecule is a protein, glycan, lipid or low molecular weight molecule.

9. The targeted lipid particle of embodiment 7, wherein the target cell is selected from the group consisting of tumor-infiltrating lymphocytes, T cells, neoplastic or tumor cells, virus-infected cells, stem cells, central nervous system (CNS) cells, hematopoeietic stem cells (HSCs), liver cells or fully differentiated cells.

10. The targeted lipid particle of embodiment 9, wherein the target cell is selected from the group consisting of a CD3+ T cell, a CD4+ Tcell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

11. The targeted lipid particle of any of the preceding embodiments, wherein the single domain antibody binds an antigen or portion thereof present on a target cell.

12. The targeted lipid particle of any of embodiments 3-11, wherein the peptide linker comprises up to 65 amino acids in length.

13. The targeted lipid particle of any of embodiments 3-11, wherein the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids.

14. The targeted lipid particle of any of embodiments 3-1 1, wherein peptide linker comprises a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 amino acids in length.

15. The targeted lipid particle of any of embodiments 3-14, wherein the peptide linker is a flexible linker that comprises GS, GGS, GGGGS (SEQ ID NO:43), GGGGGS (SEQ ID NO:41) or combinations thereof.

16. The targeted lipid particle of any of embodiments 3-15, wherein the peptide linker comprises (GGS)n, wherein n is 1 to 10.

17. The targeted lipid particle of any of embodiments 3-15, wherein the peptide linker comprises (GGGGS)n (SEQ ID NO:42), wherein n is 1 to 10.

18. The targeted lipid particle of any of embodiments 3-15, wherein the peptide linker comprises (GGGGGS)n (SEQ ID NO:27), wherein n is 1 to 6.

19. The targeted lipid particle of any of embodiments 1-18, wherein the G protein or the biologically active portion thereof is a wild-type Nipah virus G (NiV-G) protein or a Hendra virus G protein.

20. The targeted lipid particle of any of embodiments 1-19, wherein the G protein or the biologically active portion thereof is a wild-type NiV-G protein or a functionally active variant or biologically active portion thereof.

21. The targeted lipid particle of embodiment 20, wherein the mutant NiV-G protein or functionally active variant or biologically active portion thereof comprises an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:9, SEQ ID NO:28 or SEQ ID NO:44.

22. The targeted lipid particle of embodiment 21, wherein the NiV-G protein is a biologically active portion that is truncated and lacks up to 40 contiguous amino acid residues at or near the N-terminus of the wild-type NiV-G protein (SEQ ID NO:9, SEQ ID NO:28 or SEQ ID NO:44).

23. The targeted lipid particle of any of embodiments 1-18, wherein the NiV-G protein is a biologically active portion that is truncated at the N-terminus of wild-type NiV-G and has the sequence set forth in any of SEQ ID NOS: 10-15, 35-40 or 45-50 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs: 10-15, 35-40 or 45-50.

24. The targeted lipid particle of any of embodiments 21-23, wherein the NiV-G protein has a 5 amino acid truncation at or near the N-terminus of the wild-type NiV-G protein (SEQ ID NO:9, SEQ ID NO:28 or SEQ ID NO:44).

25. The targeted lipid particle of embodiment 24, wherein the NiV-G protein has the amino acid sequence set forth in SEQ ID NO: 10 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:10.

26. The targeted lipid particle of embodiment 24, wherein the NiV-G protein has the amino acid sequence set forth in SEQ ID NO: 35 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:35.

27. The targeted lipid particle of embodiment 24, wherein the NiV-G protein has the amino acid sequence set forth in SEQ ID NO: 45 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:45.

28. The targeted lipid particle of any of embodiments 21-23, wherein the NiV-G protein has a 10 amino acid truncation at or near the N-terminus of the wild-type NiV-G protein (SEQ ID NO:9, SEQ ID NO:28 or SEQ ID NO:44).

29. The targeted lipid particle of embodiment 28, wherein the NiV-G protein has the amino acid sequence set forth in SEQ ID NO: 11 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:11.

30. The targeted lipid particle of embodiment 28, wherein the NiV-G protein has the amino acid sequence set forth in SEQ ID NO: 36 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:36.

31. The targeted lipid particle of embodiment 28, wherein the NiV-G protein has the amino acid sequence set forth in SEQ ID NO: 46 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46.

32. The targeted lipid particle of any of embodiments 21-23, wherein the NiV-G protein has a 15 amino acid truncation at or near the N-terminus of the wild-type NiV-G protein (SEQ ID NO:9, SEQ ID NO:28 or SEQ ID NO:44).

33. The targeted lipid particle of embodiment 32, wherein the NiV-G protein has the amino acid sequence set forth in SEQ ID NO: 12 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12.

34. The targeted lipid particle of embodiment 32, wherein the NiV-G protein has the amino acid sequence set forth in SEQ ID NO: 37 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:37.

35. The targeted lipid particle of embodiment 32, wherein the NiV-G protein has the amino acid sequence set forth in SEQ ID NO: 47 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:47.

36. The targeted lipid particle of any of embodiments 21-23, wherein the NiV-G protein has a 20 amino acid truncation at or near the N-terminus of the wild-type NiV-G protein (SEQ ID NO:9, SEQ ID NO:28 or SEQ ID NO:44).

37. The targeted lipid particle of embodiment 36, wherein the NiV-G protein has the amino acid sequence set forth in SEQ ID NO: 13 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:13.

38. The targeted lipid particle of embodiment 36, wherein the NiV-G protein has the amino acid sequence set forth in SEQ ID NO: 38 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:38.

39. The targeted lipid particle of embodiment 36, wherein the NiV-G protein has the amino acid sequence set forth in SEQ ID NO: 48 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:48.

40. The targeted lipid particle of any of embodiments 21-23, wherein the NiV-G protein has a 25 amino acid truncation at or near the N-terminus of the wild-type NiV-G protein (SEQ ID NO:9, SEQ ID NO:28 or SEQ ID NO:44).

41. The targeted lipid particle of embodiment 40, wherein the NiV-G protein has the amino acid sequence set forth in SEQ ID NO: 14 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14.

42. The targeted lipid particle of embodiment 40, wherein the NiV-G protein has the amino acid sequence set forth in SEQ ID NO: 39 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:39.

43. The targeted lipid particle of embodiment 40, wherein the NiV-G protein has the amino acid sequence set forth in SEQ ID NO: 49 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:49.

44. The targeted lipid particle of any of embodiments 21-23, wherein the NiV-G protein has a 30 amino acid truncation at or near the N-terminus of the wild-type NiV-G protein (SEQ ID NO:9, SEQ ID NO:28 or SEQ ID NO:44).

45. The targeted lipid particle of embodiment 44, wherein the NiV-G protein has the amino acid sequence set forth in SEQ ID NO: 15 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15.

46. The targeted lipid particle of embodiment 44, wherein the NiV-G protein has the amino acid sequence set forth in SEQ ID NO: 40 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:40.

47. The targeted lipid particle of embodiment 44, wherein the NiV-G protein has the amino acid sequence set forth in SEQ ID NO: 50 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:50.

48. The targeted lipid particle of any of embodiments 21-23, wherein the NiV-G protein has a 34 amino acid truncation at or near the N-terminus of the wild-type NiV-G protein (SEQ ID NO:9, SEQ ID NO:28 or SEQ ID NO:44).

49. The targeted lipid particle of embodiment 48, wherein the NiV-G protein has the amino acid sequence set forth in SEQ ID NO: 22 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:22.

50. The targeted lipid particle of embodiment 48, wherein the NiV-G protein has the amino acid sequence set forth in SEQ ID NO: 53 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:53.

51. The targeted lipid particle any of embodiments 1-48, wherein the G-protein or the biologically active portion thereof is a mutant NiV-G protein that exhibits reduced binding to Ephrin B2 or Ephrin B3.

52. The targeted lipid particle of embodiment 51, wherein the mutant NiV-G protein comprises:

one or more amino acid substitutions corresponding to amino acid substitutions selected from the group consisting of E501A, W504A, Q530A and E533A with reference to numbering set forth in SEQ ID NO:28.

53. The targeted lipid particle of embodiment 51 or embodiment 52, wherein the mutant NiV-G protein has the amino acid sequence set forth in SEQ ID NO: 16 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:16.

54. The targeted lipid particle of embodiment 51 or embodiment 52, wherein the mutant NiV-G protein has the amino acid sequence set forth in SEQ ID NO: 51 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:51.

55. The targeted lipid particle of any of embodiments 1-54, wherein the F protein or the biologically active portion thereof is a wild-type Nipah virus F (NiV-F) protein or a Hendra virus F protein or is a functionally active variant or biologically active portion thereof.

56. The targeted lipid particle of any of embodiments 1-55, wherein the F protein or the biologically active portion thereof is a wild-type NiV-F protein or a functionally active variant or a biologically active portion thereof.

57. The targeted lipid particle of any of embodiments 1-56, wherein the NiV-F-protein or the functionally active variant or biologically active portion thereof comprises the amino acid sequence set forth in SEQ ID NO: 2, or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 2.

58. The targeted lipid particle of any of embodiments 1-57, wherein the NiV-F protein is a is a biologically active portion thereof that has a 20 amino acid truncation at or near the C-terminus of the wild-type NiV-F protein (SEQ ID NO:2).

59. The targeted lipid particle of embodiment 58, wherein the NiV-F protein has an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 5.

60. The targeted lipid particle of any of embodiments 1-57, wherein the NiV-F protein is a biologically active portion thereof that comprises:

i) a 20 amino acid truncation at or near the C-terminus of the wild-type NiV-F protein (SEQ ID NO:2); and

ii) a point mutation on an N-linked glycosylation site.

61. The targeted lipid particle of embodiment 60, wherein the NiV-F protein has an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 7.

62. The targeted lipid particle of any of embodiments 1-57, wherein the NiV-F protein is a biologically active portion thereof that has a 22 amino acid truncation at or near the C-terminus of the wild-type NiV-F protein (SEQ ID NO:2).

63. The targeted lipid particle of embodiment 62, wherein the NiV-F protein has an amino acid sequence that is encoded by a sequence of nucleotides encoding a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 8.

64. The targeted lipid particle of embodiment 63, wherein the NiV-F protein has an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 23.

65. The targeted lipid particle of any of embodiments 1-57, wherein the F-protein or the biologically active portion thereof comprises an F1 subunit or a fusogenic portion thereof.

66. The targeted lipid particle of embodiment 65, wherein the F1 subunit is a proteolytically cleaved portion of the F0 precursor.

67. The targeted lipid particle of embodiment 66, wherein the F1 subunit comprises the sequence set forth in SEQ ID NO: 4, or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 4.

68. The targeted lipid particle of any of embodiments 1-67, wherein the lipid bilayer is derived from a membrane of a host cell used for producing a retrovirus or retrovirus-like particle.

69. The targeted lipid particle of any of embodiments 1-60, wherein the lipid bilayer is or comprises a viral envelope.

70. The targeted lipid particle of embodiment 68, wherein the retrovirus-like particle is replication defective.

71. The targeted lipid particle of any of embodiments 1-70, wherein the targeted lipid particle comprises one or more viral components other than the F protein molecule and the G protein.

72. The targeted lipid particle of embodiment 71, wherein the one or more viral components are from a retrovirus.

73. The targeted lipid particle of embodiment 72, wherein the retrovirus is a lentivirus.

74. The targeted lipid particle of any of embodiments 71-73, wherein the one or more viral components comprise a viral packaging protein selected from one or more of Gag, Pol, Rev and Tat.

75. The targeted lipid particle of any of embodiments 71-74, wherein the one or more viral components comprises one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

76. The targeted lipid particle of any of embodiments 1-75, wherein the lipid particle further comprises an exogenous agent.

77. The targeted lipid particle of embodiment 76, wherein the exogenous agent is present in the lumen.

78. The targeted lipid particle of embodiment 77, wherein the exogenous agent is a protein or a nucleic acid, optionally wherein the nucleic acid is a DNA or RNA.

79. The targeted lipid particle of any of embodiments 76-78, wherein the exogenous agent encodes a therapeutic agent or a diagnostic agent.

80. The targeted lipid particle of any of embodiments 68-79, wherein the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRCS cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

81. The targeted lipid particle of any of embodiments 68-80, wherein the host cell comprises 293T cells.

82. A polynucleotide comprising a nucleic acid sequence encoding (i) a henipavirus envelope attachment glycoprotein G (G protein) or a biologically active portion thereof and (ii) a single domain antibody (sdAb) variable domain, wherein the sdAb variable domain is attached to the C-terminus of the G protein or the biologically active portion thereof.

83. The polynucleotide of embodiment 82, further comprising (iii) a nucleic acid sequence encoding a henipavirus F protein molecule or a biologically active portion thereof.

84. The polynucleotide of embodiment 82 or embodiment 83, further comprising at least one promoter that is operatively linked to control expression of the nucleic acid.

85. The polynucleotide of any of embodiments 83-84, wherein the promoter is a constitutive promoter.

86. The polynucleotide of any of embodiments 83-85, wherein the promoter is an inducible promoter.

87. The polynucleotide of any of embodiments 82-86, wherein the sdAb variable domain is attached to the G protein via an encoded peptide linker.

88. The polynucleotide of any of embodiments 86-87, wherein the encoded peptide linker comprises up to 65 amino acids in length.

89. The polynucleotide of any of embodiments 86-87, wherein the encoded peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids.

90. The polynucleotide of any of embodiments 86-87, wherein the encoded peptide linker comprises a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 amino acids in length.

91. The polynucleotide of any of embodiments 86-87, wherein the encoded peptide linker comprises GS, GGS, GGGGS (SEQ ID NO:43), GGGGGS (SEQ ID NO:41) and combinations thereof.

92. The polynucleotide of any of embodiments 86-87, wherein the encoded peptide linker comprises (GGS)n, wherein n is 1 to 10.

93. The polynucleotide of any of embodiments 86-87, wherein the encoded peptide linker comprises (GGGGS)n (SEQ ID NO:42), wherein n is 1 to 10. 94. The polynucleotide of any of embodiments 86-87, wherein the encoded peptide linker comprises (GGGGGS)n (SEQ ID NO:27), wherein n is 1 to 4.

95. The polynucleotide of any of embodiments 86-87, wherein the nucleic acid sequence encoding the G protein is a wild-type Nipah virus G (NiV-G) protein or a Hendra virus G protein or is a variant thereof that exhibits reduced binding for the native binding partner.

96. The polynucleotide of any of embodiments 82-95, wherein the nucleic acid sequence encoding the G protein is a wild-type NiV-G protein.

97. The polynucleotide of any of embodiments 82-95, wherein the nucleic acid sequence encoding the G-protein is a mutant NiV-G protein that exhibits reduced binding to Ephrin B2 or Ephrin B3.

98. The polynucleotide of embodiment 97, wherein the nucleic acid sequence encoding the mutant NiV-G protein comprises an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 9, SEQ ID NO:28 or SEQ ID NO: 44.

99. The polynucleotide of any of embodiments 82-95 and 97, wherein the nucleic acid sequence encoding the mutant NiV-G protein comprises the sequence set forth in any of SEQ ID NOS: 10-15, 35-40 or 45-50 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs: 10-15, 35-40 or 45-50.

100. The polynucleotide of any of embodiments 97-99, wherein the nucleic acid sequence encoding the mutant NiV-G protein comprises a 5 amino acid truncation at or near the N-terminus of the wild-type NiV-G protein (SEQ ID NO: 9, SEQ ID NO:28 or SEQ ID NO: 44).

101. The polynucleotide of embodiment 100, wherein the nucleic acid sequence encoding the mutant NiV-G protein comprises the amino acid sequence set forth in SEQ ID NO: 10 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:10.

102. The polynucleotide of embodiment 100, wherein the nucleic acid sequence encoding the mutant NiV-G protein comprises the amino acid sequence set forth in SEQ ID NO: 35 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:35.

103. The polynucleotide of embodiment 100, wherein the nucleic acid sequence encoding the mutant NiV-G protein comprises the amino acid sequence set forth in SEQ ID NO: 45 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:45.

104. The polynucleotide of any of embodiments 97-99, wherein the nucleic acid sequence encoding the mutant NiV-G protein comprises a 10 amino acid truncation at or near the N-terminus of the wild-type NiV-G protein (SEQ ID NO: 9, SEQ ID NO:28 or SEQ ID NO: 44).

105. The polynucleotide of embodiment 104, wherein the nucleic acid sequence encoding the mutant NiV-G protein comprises the amino acid sequence set forth in SEQ ID NO: 11 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:11.

106. The polynucleotide of embodiment 104, wherein the nucleic acid sequence encoding the mutant NiV-G protein comprises the amino acid sequence set forth in SEQ ID NO: 36 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:36.

107. The polynucleotide of embodiment 104, wherein the nucleic acid sequence encoding the mutant NiV-G protein comprises the amino acid sequence set forth in SEQ ID NO: 46 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46.

108. The polynucleotide of any of embodiments 97-99, wherein the nucleic acid sequence encoding the mutant NiV-G protein comprises a 15 amino acid truncation at or near the N-terminus of the wild-type NiV-G protein (SEQ ID NO: 9, SEQ ID NO:28 or SEQ ID NO: 44).

109. The polynucleotide of embodiment 108, wherein the nucleic acid sequence encoding the mutant NiV-G protein comprises the amino acid sequence set forth in SEQ ID NO: 12 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12.

110. The polynucleotide of embodiment 108, wherein the nucleic acid sequence encoding the mutant NiV-G protein comprises the amino acid sequence set forth in SEQ ID NO: 37 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:37.

111. The polynucleotide of embodiment 108, wherein the nucleic acid sequence encoding the mutant NiV-G protein comprises the amino acid sequence set forth in SEQ ID NO: 47 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:47.

112. The polynucleotide of any of embodiments 97-99, wherein the nucleic acid sequence encoding the mutant NiV-G protein comprises a 20 amino acid truncation at or near the N-terminus of the wild-type NiV-G protein (SEQ ID NO: 9, SEQ ID NO:28 or SEQ ID NO: 44).

113. The polynucleotide of embodiment 112, wherein the nucleic acid sequence encoding the mutant NiV-G protein comprises the amino acid sequence set forth in SEQ ID NO: 13 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:13.

114. The polynucleotide of embodiment 112, wherein the nucleic acid sequence encoding the mutant NiV-G protein comprises the amino acid sequence set forth in SEQ ID NO: 38 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:38.

115. The polynucleotide of embodiment 112, wherein the nucleic acid sequence encoding the mutant NiV-G protein comprises the amino acid sequence set forth in SEQ ID NO: 48 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:48.

116. The polynucleotide of any of embodiments 97-99, wherein the nucleic acid sequence encoding the mutant NiV-G protein comprises a 25 amino acid truncation at or near the N-terminus of the wild-type NiV-G protein (SEQ ID NO: 9, SEQ ID NO:28 or SEQ ID NO: 44).

117. The polynucleotide of embodiment 116, wherein the nucleic acid sequence encoding the mutant NiV-G protein comprises the amino acid sequence set forth in SEQ ID NO: 14 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14.

118. The polynucleotide of embodiment 116, wherein the nucleic acid sequence encoding the mutant NiV-G protein comprises the amino acid sequence set forth in SEQ ID NO: 39 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:39.

119. The polynucleotide of embodiment 116, wherein the nucleic acid sequence encoding the mutant NiV-G protein comprises the amino acid sequence set forth in SEQ ID NO: 49 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:49.

120. The polynucleotide of any of embodiments 97-99, wherein the nucleic acid sequence encoding the mutant NiV-G protein comprises a 30 amino acid truncation at or near the N-terminus of the wild-type NiV-G protein (SEQ ID NO: 9, SEQ ID NO:28 or SEQ ID NO: 44).

121. The polynucleotide of embodiment 120, wherein the nucleic acid sequence encoding the mutant NiV-G protein comprises the amino acid sequence set forth in SEQ ID NO: 15 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15.

122. The polynucleotide of embodiment 120, wherein the nucleic acid sequence encoding the mutant NiV-G protein comprises the amino acid sequence set forth in SEQ ID NO: 40 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:40.

123. The polynucleotide of embodiment 120, wherein the nucleic acid sequence encoding the mutant NiV-G protein comprises the amino acid sequence set forth in SEQ ID NO: 50 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 50.

124. The polynucleotide of any of embodiments 97-99, wherein the nucleic acid sequence encoding the mutant NiV-G protein comprises:

i) a truncation at or near the N-terminus; and

ii) point mutations selected from the group consisting of E501A, W504A, Q530A and E533A.

125. The polynucleotide of embodiment 124, wherein the nucleic acid sequence encoding the mutant NiV-G protein comprises the amino acid sequence set forth in SEQ ID NO: 16 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:16.

126. The polynucleotide of embodiment 124, wherein the nucleic acid sequence encoding the mutant NiV-G protein comprises the amino acid sequence set forth in SEQ ID NO: 51 or an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:51.

127. A vector, comprising the polynucleotide of any of embodiments 82-126.

128. The vector of embodiment 127, wherein the vector is a mammalian vector, viral vector or artificial chromosome, optionally wherein the artificial chromosome is a bacterial artificial chromosome (BAC).

129. A cell comprising the polynucleotide of any of embodiments 82-126 or the vector of embodiment 127 or embodiment 128.

130. A method of making a targeted lipid particle comprising a henipavirus F protein molecule or biologically active portion thereof and a targeted envelope protein comprising a henipavirus envelope attachment glycoprotein G (G protein) or a biologically active portion thereof and a single domain antibody (sdAb) variable domain comprising:

a) providing a cell that comprises a nucleic acid encoding a henipavirus F protein molecule or biologically active portion thereof and a nucleic acid encoding a targeted envelope protein comprising a henipavirus envelope attachment glycoprotein G (G protein) or a biologically active portion thereof and a single domain antibody (sdAb) variable domain;

b) culturing the cell under conditions that allow for production of a targeted lipid particle, and

c) separating, enriching, or purifying the targeted lipid particle from the cell, thereby making the targeted lipid particle.

131. A method of making a targeted lipid particle comprising a henipavirus F protein molecule or biologically active portion thereof and a targeted envelope protein comprising a henipavirus envelope attachment glycoprotein G (G protein) or a biologically active portion thereof and a single domain antibody (sdAb) variable domain, comprising:

a) providing a cell that comprises the polynucleotide of any of embodiments 82-126 or the vector of embodiment 127 or embodiment 128;

b) providing the cell a polynucleotide encoding a henipavirus F protein molecule or biologically active portion thereof;

c) culturing the cell under conditions that allow for production of a targeted lipid particle, and

d) separating, enriching, or purifying the targeted lipid particle particle from the cell, thereby making the targeted lipid particle.

132. The method of embodiment 130 or embodiment 131, wherein the cell is a mammalian cell.

133. The method of any of embodiments 130-131, wherein the cell is a producer cell and the targeted lipid particle is a viral particle or a viral-like particle, optionally a retroviral particle or a retroviral-like particle, optionally a lentiviral particle or lentiviral-like particle.

134. A producer cell comprising (i) a viral nucleic acid(s) and (ii) nucleic acid encoding a henipavirus F protein molecule or biologically active portion thereof and (iii) a nucleic acid encoding a targeted envelope protein comprising a henipavirus envelope attachment glycoprotein G (G protein) or a biologically active portion thereof and a single domain antibody (sdAb) variable domain, optionally wherein the viral nucleic acid(s) are lentiviral nucleic acids.

135. The producer cell of embodiment 134, wherein the viral nucleic acid(s) lacks one or more genes involved in viral replication.

136. The producer cell of embodiment 134 or embodiment 135, wherein the viral nucleic acid comprises a nucleic acid encoding a viral packaging protein selected from one or more of Gag, Pol, Rev and Tat.

137. The producer cell of any of embodiments 134-136, wherein the viral nucleic acid comprises:

one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3);

138. The producer cell of any of embodiments 134-137, wherein the henipavirus F protein molecule or biologically active portion thereof comprises:

(i) the sequence set forth in SEQ ID NO: 2;

(ii) an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:2.

139. The producer cell of any of embodiments 134-137, wherein the henipavirus F protein molecule or biologically active portion thereof comprises:

(i) the sequence set forth in SEQ ID NO: 5;

(ii) an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:5.

140. The producer cell of any of embodiments 134-137, wherein the henipavirus F protein molecule or biologically active portion thereof comprises:

(i) the sequence set forth in SEQ ID NO: 7;

(ii) an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:7.

141. The producer cell of any of embodiments 134-137, wherein the henipavirus F protein molecule or biologically active portion thereof comprises:

(i) a sequence encoding by a nucleotide sequence encoding the sequence set forth in SEQ ID NO: 8;

(ii) a amino acid sequence encoded by a nucleotide sequence encoding a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:8.

142. The producer cell of any of embodiments 134-137, wherein the henipavirus F protein molecule or biologically active portion thereof comprises:

(i) the sequence set forth in SEQ ID NO: 23;

(ii) an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:23.

143. The producer cell of any of embodiments 134-142, wherein the henipavirus envelope attachment glycoprotein G (G protein) or a biologically active portion thereof comprises:

(i) the sequence set forth in SEQ ID NO: 9, SEQ ID NO:28 or SEQ ID NO:44;

(ii) an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO: 9, SEQ ID NO:28 or SEQ ID NO:44.

144. The producer cell of any of embodiments 134-142, wherein the henipavirus envelope attachment glycoprotein G (G protein) or a biologically active portion thereof comprises:

(i) the sequence set forth in SEQ ID NO: 10;

(ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:10.

145. The producer cell of any of embodiments 134-142, wherein the henipavirus envelope attachment glycoprotein G (G protein) or a biologically active portion thereof comprises:

(i) the sequence set forth in SEQ ID NO: 35;

(ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:35.

146. The producer cell of any of embodiments 134-142, wherein the henipavirus envelope attachment glycoprotein G (G protein) or a biologically active portion thereof comprises:

(i) the sequence set forth in SEQ ID NO: 45;

(ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:45.

147. The producer cell of any of embodiments 134-142, wherein the henipavirus envelope attachment glycoprotein G (G protein) or a biologically active portion thereof comprises:

(i) the sequence set forth in SEQ ID NO: 11;

(ii) an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:11.

148. The producer cell of any of embodiments 134-142, wherein the henipavirus envelope attachment glycoprotein G (G protein) or a biologically active portion thereof comprises:

(i) the sequence set forth in SEQ ID NO: 36;

(ii) an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:36.

149. The producer cell of any of embodiments 134-142, wherein the henipavirus envelope attachment glycoprotein G (G protein) or a biologically active portion thereof comprises:

(i) the sequence set forth in SEQ ID NO: 46;

(ii) an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:46.

150. The producer cell of any of embodiments 134-142, wherein the henipavirus envelope attachment glycoprotein G (G protein) or a biologically active portion thereof comprises:

(i) the sequence set forth in SEQ ID NO: 12;

(ii) an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:12.

151. The producer cell of any of embodiments 134-142, wherein the henipavirus envelope attachment glycoprotein G (G protein) or a biologically active portion thereof comprises:

(i) the sequence set forth in SEQ ID NO: 37;

(ii) an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:37.

152. The producer cell of any of embodiments 134-142, wherein the henipavirus envelope attachment glycoprotein G (G protein) or a biologically active portion thereof comprises:

(i) the sequence set forth in SEQ ID NO: 47;

(ii) an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:47.

153. The producer cell of any of embodiments 134-142, wherein the henipavirus envelope attachment glycoprotein G (G protein) or a biologically active portion thereof comprises:

(i) the sequence set forth in SEQ ID NO: 13;

(ii) an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:13.

154. The producer cell of any of embodiments 134-142, wherein the henipavirus envelope attachment glycoprotein G (G protein) or a biologically active portion thereof comprises:

(i) the sequence set forth in SEQ ID NO: 38;

(ii) an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:38.

155. The producer cell of any of embodiments 134-142, wherein the henipavirus envelope attachment glycoprotein G (G protein) or a biologically active portion thereof comprises:

(i) the sequence set forth in SEQ ID NO: 48;

(ii) an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:48.

156. The producer cell of any of embodiments 134-142, wherein the henipavirus envelope attachment glycoprotein G (G protein) or a biologically active portion thereof comprises:

(i) the sequence set forth in SEQ ID NO: 14;

(ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:14.

157. The producer cell of any of embodiments 134-142, wherein the henipavirus envelope attachment glycoprotein G (G protein) or a biologically active portion thereof comprises:

(i) the sequence set forth in SEQ ID NO: 39;

(ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:39.

158. The producer cell of any of embodiments 134-142, wherein the henipavirus envelope attachment glycoprotein G (G protein) or a biologically active portion thereof comprises:

(i) the sequence set forth in SEQ ID NO: 49;

(ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:49.

159. The producer cell of any of embodiments 134-142, wherein the henipavirus envelope attachment glycoprotein G (G protein) or a biologically active portion thereof comprises:

(i) the sequence set forth in SEQ ID NO: 15;

(ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:15.

160. The producer cell of any of embodiments 134-142, wherein the henipavirus envelope attachment glycoprotein G (G protein) or a biologically active portion thereof comprises:

(i) the sequence set forth in SEQ ID NO: 40;

(ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:40.

161. The producer cell of any of embodiments 134-142, wherein the henipavirus envelope attachment glycoprotein G (G protein) or a biologically active portion thereof comprises:

(i) the sequence set forth in SEQ ID NO: 50;

(ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:50.

162. The producer cell of any of embodiments 134-142, wherein the henipavirus envelope attachment glycoprotein G (G protein) or a biologically active portion thereof comprises:

(i) the sequence set forth in SEQ ID NO: 16;

(ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:16.

163. The producer cell of any of embodiments 134-142, wherein the henipavirus envelope attachment glycoprotein G (G protein) or a biologically active portion thereof comprises:

(i) the sequence set forth in SEQ ID NO: 51;

(ii) an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:51.

164. A viral vector particle or viral-like particle produced from the producer cell of any of embodiments 134-163.

165. A composition comprising a plurality of targeted lipid particles of any of embodiments 1-81 and 173-176.

166. The composition of embodiment 165 further comprising a pharmaceutically acceptable carrier.

167. The pharmaceutical composition of embodiment 165 or embodiment 166, wherein the targeted lipid particles comprise an average diameter of less than 1 μm.

168. A method of delivering an exogenous agent to a subject (e.g., a human subject), the method comprising administering to the subject the targeted lipid particle of any of embodiments 1-81 and 173-176 or the composition of any of embodiments 165-167 and 177.

169. A method of treating a disease or disorder in a subject (e.g., a human subject), the method comprising administering to the subject a targeted lipid particle of any of embodiments 1-81 and 173-176 or the composition of any of embodiments 165-167 and 177.

170. A method of fusing a mammalian cell to a targeted lipid particle, the method comprising administering to the subject a targeted lipid particle of any of embodiments 1-81 and 173-176 or the composition of any of embodiments 165-167 and 177.

171. The method of embodiment 170, wherein the fusing of the mammalian cell to the targeted lipid particle delivers an exogenous agent to a subject (e.g., a human subject).

172. The method of embodiment 170 or embodiment 171, wherein the fusing of the mammalian cell to the targeted lipid particle treats a disease or disorder in a subject (e.g., a human subject).

173. The targeted lipid particle of any of embodiments 1-81, wherein the targeted lipid particle has greater expression of the targeted envelope protein compared to a reference lipid particle that has incorporated into a similar lipid bilayer the same envelope protein but that is fused to an alternative targeting moiety, optionally wherein the alternative targeting moiety is a single chain variable fragment (scFv).

174. The targeted lipid particle of embodiment 173, wherein the expression is increased by at or greater than 5%, 10%, 20%, 30%, 40%, 50%, 60%, 70%, 80%, 90%, 100%, 125%, 150%, 200%, 300%, 400%, 500% or more.

175. The targeted lipid particle of embodiment 173, wherein the expression is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, preferably at or about or greater than 10-fold or more.

176. The targeted lipid particle of any of embodiments 1-81 and 173-175 or the viral vector particle or viral-like particle of embodiment 164, wherein the titer in target cells following transduction is at or greater than 1×10⁶ transduction units (TU)/mL, at or greater than 2×10⁶ TU/mL, at or greater than 3×10⁶ TU/mL, at or greater than 4×10⁶ TU/mL, at or greater than 5×10⁶ TU/mL, at or greater than 6×10⁶ TU/mL, at or greater than 7×10⁶ TU/mL, at or greater than 8×10⁶ TU/mL, at or greater than 9×10⁶ TU/mL, or at or greater than 1×10⁷ TU/mL.

177. The composition of any of embodiments 165-167, wherein among the population of lipid particles in the composition, greater than at or about 50%, greater than at or about 55%, greater than at or about 60%, greater than at or about 65%, greater than at or about 70%, or greater than at or about 75% are surface positive for the targeted envelope protein.

178. The targeted lipid particle of any of embodiments 1-81 and 173-176, wherein the targeted envelope protein is present on the surface of the targeted lipid particle at a density of at least about (0.001, 0.002, 0.005, 0.01, 0.02, 0.05, 0.1, 0.2 or 0.5) targeted envelope proteins/nm².

179. A composition comprising a plurality of the targeted lipid particles of any of embodiments 1-81, 173-176 and 178, wherein the targeted envelope protein is present on the surface of the targeted lipid particles at an average density of at least about (0.001, 0.002, 0.005, 0.01, 0.02, 0.05, 0.1, 0.2 or 0.5) targeted envelope proteins/nm².

180. The producer cell of any one of embodiments 134-163, wherein the producer cell has greater membrane (e.g., plasma membrane) expression of the targeted envelope protein compared to a reference producer cell that has incorporated into its membrane (e.g. plasma membrane) the same envelope protein but that is fused to an alternative targeting moiety, optionally wherein the alternative targeting moiety is a single chain variable fragment (scFv).

181. The producer cell of embodiment 180, wherein the expression is increased by at or greater than 5%, 10%, 20%, 30%, 40%, 50%, 60%, 70%, 80%, 90%, 100%, 125%, 150%, 200%, 300%, 400%, 500% or more.

182. The producer cell of embodiment 180, wherein the expression is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, preferably at or about or greater than 10-fold or more.

183. The producer cell of any one of embodiments 134-163 and 180-182, wherein the producer cell has the expression of the targeted envelope protein on a membrane (e.g., plasma membrane) of the producer cell is at least 20 proteins (e.g., at least 50, 100, 200, 500, 1000, 2000, 5000, or 10,000 proteins) per square micron.

184. The producer cell of any one of embodiments 134-163 and 180-183, wherein the targeted envelope protein comprises at least 0.1% (e.g., at least 0.2%, 0.5%, 1%, 2%, 3%, 4%, 5%, 6%, 7%, 8%, 9%, or 10%) of the total membrane (e.g., plasma membrane) proteins of the producer cell (e.g., by total protein weight).

### EXAMPLES

The following examples are included for illustrative purposes only and are not intended to limit the scope of the invention.

### Example 1: Generation and Characterization of Producer Cells Containing Targeted Binders

This Example describes generation and assessment of NiVG targeted binding sequences in which NiVG was linked to scFv or VHH binding modalities.

### A. Binding Modalities Directed to CD4.

Exemplary retargeted NivG fusogen constructs were generated containing an scFv or VHH binding modality against human cellular receptor CD4. For each binding modality, four different sequences that contained a unique CDR3 were assessed. Each exemplary binder sequence was codon optimized and cloned into an expression vector as a fusion with a sequence encoding NiVG (GcΔ34; Bender et al. 2016 PLoS Pathol 12(6):e1005641). The resulting vectors encoded a NivG targeting domain containing NiVG (SEQ ID NO:16) a flexible linker and the binding domain, followed by a 6xHis-tag for detection (NivG-linker-scFv-6xHis).

After subcloning, 5 μg of each exemplary construct was transfected into HEK 293 cells using a transfection reagent. A pcDNA3.1 plasmid (empty vector) and the expression vector without the binder domain (NiVG-linker-NoBinder) were used as negative controls.

At 48 hours post-transfection, cells were harvested and 100,000 cells were incubated for 1 hour at 4° C. with either 50 nM or 300 nM of soluble human CD4 protein with a human Fc tag (hCD4-Fc). After incubation, cells were washed and co-stained with an anti-His antibody conjugated to Alexa-647 to detect surface expression of NivG-binders and an anti-human Fc antibody conjugated to Alexa-488 to detect binding to soluble hCD4-Fc protein.

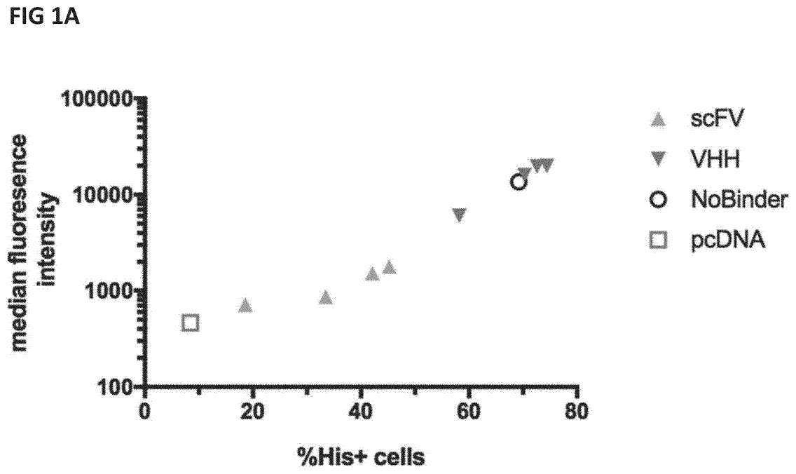

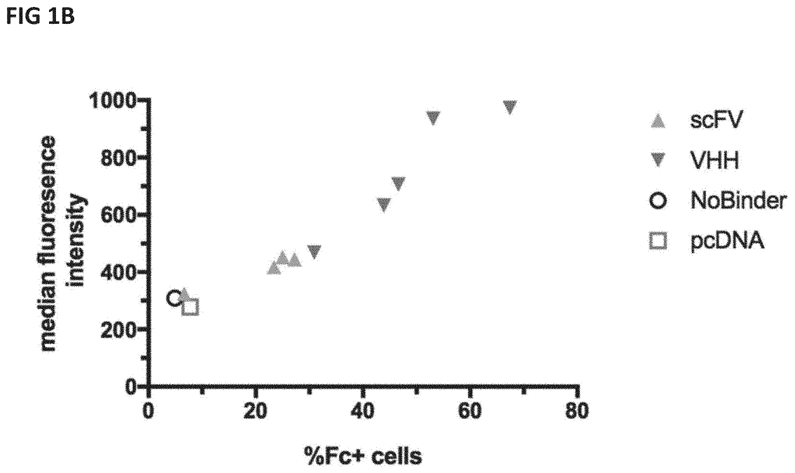

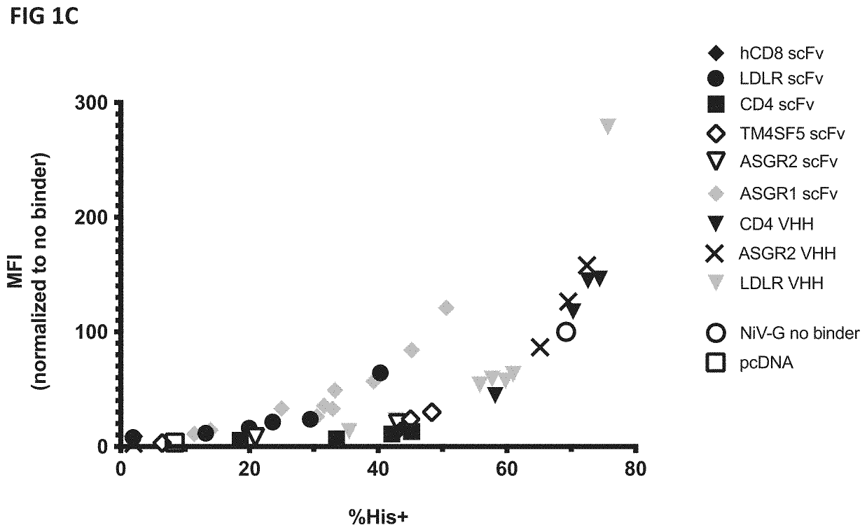

Cells were analyzed by flow cytometry, and gates for His (surface expression) and Fc (CD4-protein binding) were set based on the negative control empty vector (pcDNA3.1). Evaluation of median fluorescence intensity (MFI) of cells transfected with constructs containing VHH binding modalities demonstrated higher surface expression as quantified by % of His+ cells (FIG. 1A) and higher binding to soluble hCD4-Fc protein as quantified by % Fc+ cell (FIG. 1B), than cells transfected with constructs containing scFv binding modalities.

### B. Binding Modalities Directed to Multiple Cellular Receptors

Exemplary constructs were generated containing scFv and VHH binding modalities generally as described above, but containing unique sequences directed against other cellular receptors hCD8, CD4, ASGR2, TM4SF5, LDLR or ASGR1. Multiple sequences, each containing a unique CDR3, were assessed for each binding modality containing distinct cellular receptors. After subcloning into the NivG-linker-6xHis expression vector as described above, 5 μg of each exemplary construct was transfected into about HEK 293 cells. The pcDNA3.1 plasmid (empty vector) and the expression vector without the binding domain (NiVG-linker-NoBinder) were used as negative controls.

At 48 hours post-transfection, cells were harvested and 100,000 cells were washed and stained with an anti-His antibody conjugated to Alexa-647 to detect surface expression of NivG-binders. Cells were analyzed by flow cytometry, and gates for His (surface expression) were set based on the negative control empty vector (pcDNA3.1). Median fluorescence intensity (MFI) was normalized to that of the NivG-NoBinder control set to 100. Cells transfected with constructs containing VHH binding modalities, compared to the scFv binding modalities, demonstrated higher surface expression of targeted binding sequences on 293 cells as quantified by % of His+ cells (FIG. 1C).

### Example 2: Generation and Characterization of Lentiviruses Pseudotyped with Targeted Binders

This Example describes generation of lentiviruses pseudotyped with NivG retargeted fusogens and assessment of transduction of primary human T cells.

### A. Generation of NivG Pseudotyped Lentiviruses.

293 cells were plated at 5.4×10⁶ into 10 cm dishes and allowed to rest for 24 hours. At 24 hours after plating, cells were transfected using polyethylenimine (PEI) with the following plasmids: NivG pseudotyped vector containing hCD4 targeted binding sequences linked to scFv or VHH binding modalities (NivG-linker-hCD4-binding modality), vector containing a nucleotide sequence encoding the NivF sequence NivFde122 (SEQ ID NO:8; or SEQ ID NO:23 without a signal sequence; Bender et al. 2016 PLoS), a packaging plasmid containing an empty backbone, an HIV-1 pol, HIV-1 gag, HIV-1 Rev, HIV-1 Tat, an AmpR promoter and an SV40 promoter and a lentiviral reporter plasmid encoding an enhanced green fluorescent protein (eGFP) under the control of a SFFV promoter pLenti-SFFV-eGFP. Positive control cells were generated using the plasmids described above along with 4 μg of VSV-G.

### B. NivG Pseudotyped Lentiviral Transduction Efficiency of Primary Human T Cells.

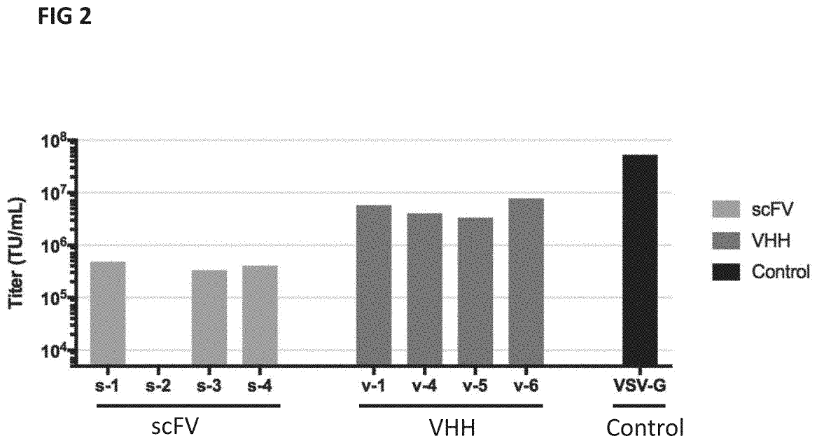

PanT cells from peripheral blood (StemCellTech, Vancouver, Canada) that were negatively selected to enrich for T cells were thawed and activated with anti CD3/anti-CD28 for 2 days. Concentrated lentiviruses generated generally as described above were serially diluted 6-fold starting at 0.05 dilution with a total of 4 points in the dilution series. Lentiviruses were added to 100,000 PanT cells and transduced by spinfection for 90 minutes at 1000 g at 25C. Transduced PanT cells were split on days 2 and 5 post-transduction, and on day 7 post-transduction, cells were harvested and stained with an Alexa-647 conjugated anti-human CD4 antibody. Cells were analyzed by flow cytometry, and titer was determined by % of CD4-positive cells that were GFP+. Cells transfected with constructs containing VHH binding modalities demonstrated a 10-fold increased titer over constructs containing scFv binding modalities on primary human T cells (FIG. 2).

### Example 3. In Vivo Delivery of Lentiviruses Pseudotyped with CD8 Targeted Binders

This Example describes generation of lentiviruses pseudotyped with a CD8 NivG retargeted fusogen and in vivo assessment of transduction of primary human T cells.

CD8 retargeted NivG fusogens were generated essentially as described in Example 2. The retargeted NivG pseudotyped fusogen contained a NivG targeting domain containing NiVG (SEQ ID NO:16) a flexible linker and an exemplary CD8 binding domain, either a VHH or scFv binding modality.

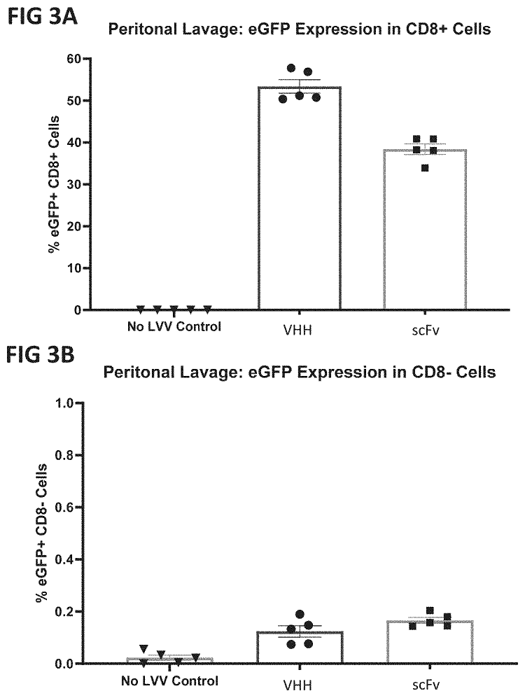

T cells from human peripheral blood mononuclear cells (PBMCs) were activated with anti CD3/anti-CD28 for 3 days. After 3 days of incubation, 1×10⁷ cells were injected intraperitoneally into NOD-scid-IL2rγ^{null} mice. One day post-injection, mice received 1×10⁷ transducing units (TU) of CD8 NivG pseudotyped lentiviruses generated as described above, or no lenti-viral vector (LVV) control, through intraperitoneal injection. On day 7 post-CD8 NivG psedudotyped lentivirus injection, peritoneal cells were harvested and analyzed by flow cytometry, and titer was determined by % of CD8 positive or negative cells that were GFP+. The CD8 retargeted pseudotyped lentiviruses demonstrated significant in vivo transduction of CD8+ T cells (FIG. 3A) and minimal transduction of CD8− T cells (FIG. 3B). These results indicate that CD8 targeted pseudotyped lentiviral-mediated delivery permits specific delivery of a transgene to the intended cell type (e.g. CD8+ T cells).

### Example 4. In Vitro Assessment of Chimeric Antigen Receptor (Car) Containing Pseudotyped Lentiviruses with CD8 Targeted Binders

This Example describes the in vitro tumor killing activity of lentivirus pseudotyped with a CD8 retargeted fusogen and expressing a CD19-directed chimeric antigen receptor (CD19CAR). The lentiviruses were generated substantially as described in Example 3, except that a plasmid encoding either the eGFP or the CD19CAR were transfected into the 293 producer cells. The CD19CAR contained an anti-scFv directed against CD19 and an intracellular signaling domain containing intracellular components of 4-1BB and CD3-zeta.

Human peripheral blood mononuclear cells (PBMCs) were activated with anti CD3/anti-CD28reagent and were transduced with CD8 retargeted NivG lentiviruses expressing CD19+CAR or GFP at various concentration ranges (10-10,000 transducing units/well). RFP+Nalm6 leukemia cells were added to cultures on day 3, and elimination of Nalm6 cells was evaluated at 18 hours by flow cytometry.

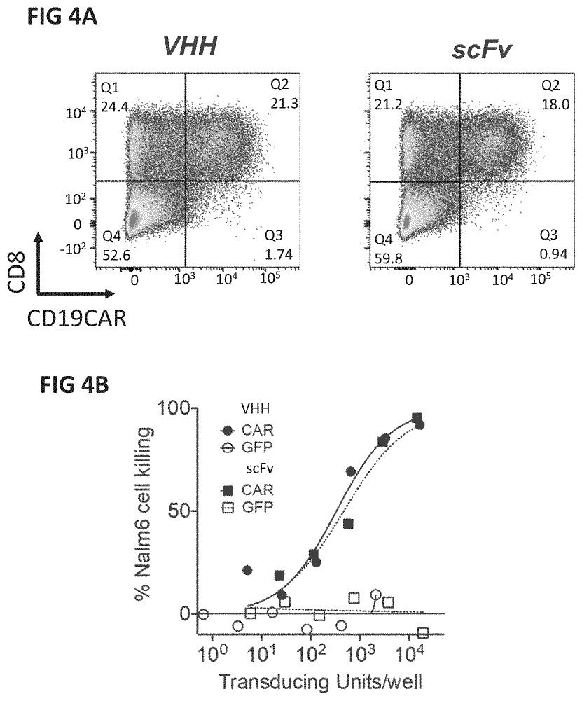

As shown in FIG. 4A, CD19+CAR expression was detected specifically in CD8+ cells with both CD8 retargeted fusogens at 4 days after transduction. Transduced CD8+ T cells expressing the CD19CAR also mediated a potent and lentivirus dose-dependent increase in killing of CD19+ Nalm6 leukemia cells, while in contrast, cells transduced to express GFP did not exhibit target cell killing (FIG. 4B).

These results demonstrate that CD8-retargeted pseudotyped lentiviruses with a transgene encoding a CD19CAR deliver CD19CAR to human CD8+ T cells to mediate a specific transduction of CD8+ T cells in a complex mixture of PBMCs and showed a dose-dependent anti-tumor response by killing of leukemic cells in vitro.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | ANNOTATION |
|---|---|---|
| 1 | MVVILDKRCY CNLLILILMI SECSVGILHY EKLSKIGLVK | Nipah virus |
| | GVTRKYKIKS NPLTKDIVIK MIPNVSNMSQ CTGSVMENYK | NiV-F with |
| | TRLNGILTPI KGALEIYKNN THDLVGDVRL AGVIMAGVAI | signal sequence |
| | GIATAAQITA GVALYEAMKN ADNINKLKSS IESTNEAVVK | (aa 1-546) |
| | LQETAEKTVY VLTALQDYIN TNLVPTIDKI SCKQTELSLD | Uniprot Q9IH63 |
| | LALSKYLSDL LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK | |
| | KRNTYSRLED RRVRPTSSGD LYYIGT | |
| 2 | ILHY EKLSKIGLVK GVTRKYKIKS NPLTKDIVIK MIPNVSNMSQ | Nipah virus |
| | CTGSVMENYK TRLNGILTPI KGALEIYKNN THDLVGDVRL | NiV-F F0 (aa 27- |
| | AGVIMAGVAI GIATAAQITA GVALYEAMKN ADNINKLKSS | 546) |
| | IESTNEAVVK LQETAEKTVY VLTALQDYIN TNLVPTIDKI | |
| | SCKQTELSLD LALSKYLSDL LFVFGPNLQD PVSNSMTIQA | |
| | ISQAFGGNYE TLLRTLGYAT EDFDDLLESD SITGQIIYVD | |
| | LSSYYIIVRV YFPILTEIQQ AYIQELLPVS FNNDNSEWIS | |
| | IVPNFILVRN TLISNIEIGF CLITKRSVIC NQDYATPMTN | |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI | |
| | TFISFIIVEK KRNTYSRLED RRVRPTSSGD LYYIGT | |
| 3 | ILHYEKLSKIGLVKGVTRKYKIKSNPLIKDIVIKMIPNVSNMSQCTGSVME | Nipah virus |
| | NYKTRLNGILTPIKGALEIYKNNTHDLVGDVR | NiV-F F2 (aa 27- |
| | | 109) |
| 4 | LAGVIMAGVAIGIATAAQITAGVALYEAMKNADNINKLKSSIESTNEAVVK | Nipah virus NiV |
| | LQETAEKTVYVLTALQDYINTNLVPTIDKISCKQTELSLDLALSKYLSDLL | F F1 (aa 110- |
| | FVFGPNLQDPVSNSMTIQAISQAFGGNYETLLRTLGYATEDFDDLLESDSI | 546) |
| | TGQIIYVDLSSYYIIVRVYFPILTEIQQAYIQELLPVSFNNDNSEWISIVP | |
| | NFILVRNTLISNIEIGFCLITKRSVICNQDYATPMTNNMRECLTGSTEKCP | |
| | RELVVSSHVPRFALSNGVLFANCISVTCQCQTTGRAISQSGEQTLLMIDNT | |
| | TCPTAVLGNVIISLGKYLGSVNYNSEGIAIGPPVFTDKVDISSQISSMNQS | |
| | LQQSKDYIKEAQRLLDTVNPSLISMLSMIILYVLSIASLCIGLITFISFII | |
| | VEKKRNTYSRLEDRRVRPTSSGDLYYIGT | |
| 5 | ILHY EKLSKIGLVK GVTRKYKIKS NPLTKDIVIK MIPNVSNMSQ | Nipah virus |
| | CTGSVMENYK TRLNGILTPI KGALEIYKNN THDLVGDVRL | NiV-F F0 T234 |
| | AGVIMAGVAI GIATAAQITA GVALYEAMKN ADNINKLKSS | truncation (aa |
| | IESTNEAVVK LQETAEKTVY VLTALQDYIN TNLVPTIDKI | 525-544) |
| | SCKQTELSLD LALSKYLSDL LFVFGPNLQD PVSNSMTIQA | |
| | ISQAFGGNYE TLLRTLGYAT EDFDDLLESD SITGQIIYVD | |
| | LSSYYIIVRV YFPILTEIQQ AYIQELLPVS FNNDNSEWIS | |
| | IVPNFILVRN TLISNIEIGF CLITKRSVIC NQDYATPMTN | |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI | |
| | TFISFIIVEK KRNTGT | |
| 6 | LAGVIMAGVAIGIATAAQITAGVALYEAMKNADNINKLKSSIESTNEAVVK | Nipah virus NiV |
| | LQETAEKTVYVLTALQDYINTNLVPTIDKISCKQTELSLDLALSKYLSDLL | F F1 (aa 110- |
| | FVFGPNLQDPVSNSMTIQAISQAFGGNYETLLRTLGYATEDFDDLLESDSI | 546) truncation |
| | TGQIIYVDLSSYYIIVRVYFPILTEIQQAYIQELLPVSFNNDNSEWISIVP | (aa 525-544) |
| | NFILVRNTLISNIEIGFCLITKRSVICNQDYATPMTNNMRECLTGSTEKCP | |
| | RELVVSSHVPRFALSNGVLFANCISVTCQCQTTGRAISQSGEQTLLMIDNT | |
| | TCPTAVLGNVIISLGKYLGSVNYNSEGIAIGPPVFTDKVDISSQISSMNQS | |
| | LQQSKDYIKEAQRLLDTVNPSLISMLSMIILYVLSIASLCIGLITFISFII | |
| | VEKKRNTGT | |
| 7 | ILHY EKLSKIGLVK GVTRKYKIKS NPLTKDIVIK MIPNVSNMSQ | Nipah virus |
| | CTGSVMENYK TRLNGILTPI KGALEIYKNQ THDLVGDVRL | NiV-F F0 T234 |
| | AGVIMAGVAI GIATAAQITA GVALYEAMKN ADNINKLKSS | truncation (aa |
| | IESTNEAVVK LQETAEKTVY VLTALQDYIN TNLVPTIDKI | 525-544) AND |
| | SCKQTELSLD LALSKYLSDL LFVFGPNLQD PVSNSMTIQA | mutation on N- |
| | ISQAFGGNYE TLLRTLGYAT EDFDDLLESD SITGQIIYVD | linked |
| | LSSYYIIVRV YFPILTEIQQ AYIQELLPVS FNNDNSEWIS | glycosylation |
| | IVPNFILVRN TLISNIEIGF CLITKRSVIC NQDYATPMTN | site |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI | |
| | TFISFIIVEK KRNTGT | |
| 8 | MVVILDKRCY CNLLILILMI SECSVGILHY EKLSKIGLVK | Truncated NiV |
| | GVTRKYKIKS NPLTKDIVIK MIPNVSNMSQ CTGSVMENYK | fusion |
| | TRLNGILTPI KGALEIYKNN THDLVGDVRL AGVIMAGVAI | glycoprotein |
| | GIATAAQITA GVALYEAMKN ADNINKLKSS IESTNEAVVK | (FcDelta22) at |
| | LQETAEKTVY VLTALQDYIN TNLVPTIDKI SCKQTELSLD | cytoplasmic tail |
| | LALSKYLSDL LFVFGPNLQD PVSNSMTIQA ISQAFGGNYE | (with signal |
| | TLLRTLGYAT EDFDDLLESD SITGQIIYVD LSSYYIIVRV | sequence) |
| | YFPILTEIQQ AYIQELLPVS FNNDNSEWIS IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNT | |
| 9 | MGPAENKKVR FENTTSDKGK IPSKVIKSYY GTMDIKKINE | NiVG protein |
| | GLLDSKILSA FNTVIALLGS IVIIVMNIMI IQNYTRSTDN | attachment |
| | QAVIKDALQG IQQQIKGLAD KIGTEIGPKV SLIDTSSTIT | glycoprotein |
| | IPANIGLLGS KISQSTASIN ENVNEKCKFT LPPLKIHECN | (602 aa) |
| | ISCPNPLPFR EYRPQTEGVS NLVGLPNNIC LQKTSNQILK | |
| | PKLISYTLPV VGQSGTCITD PLLAMDEGYF AYSHLERIGS | |
| | CSRGVSKQRI IGVGEVLDRG DEVPSLFMTN VWTPPNPNTV | |
| | YHCSAVYNNE FYYVLCAVST VGDPILNSTY WSGSLMMTRL | |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRFNTC PEICWEGVYN DAFLIDRINW | |
| | ISAGVFLDSN QTAENPVFTV FKDNEILYRA QLASEDTNAQ | |
| | KTITNCFLLK NKIWCISLVE IYDTGDNVIR PKLFAVKIPE QC | |
| 10 | MGKVR FENTTSDKGK IPSKVIKSYY GTMDIKKINE GLLDSKILSA | NiVG protein |
| | FNTVIALLGS IVIIVMNIMI IQNYTRSTDN QAVIKDALQG | attachment |
| | IQQQIKGLAD KIGTEIGPKV SLIDTSSTIT IPANIGLLGS | glycoprotein |
| | KISQSTASIN ENVNEKCKFT LPPLKIHECN ISCPNPLPFR | Truncated Δ5 |
| | EYRPQTEGVS NLVGLPNNIC LQKTSNQILK PKLISYTLPV | |
| | VGQSGTCITD PLLAMDEGYF AYSHLERIGS CSRGVSKQRI | |
| | IGVGEVLDRG DEVPSLFMTN VWTPPNPNTV YHCSAVYNNE | |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEILYRA QLASEDTNAQ KTITNCFLLK | |
| | NKIWCISLVE IYDTGDNVIR PKLFAVKIPE QC | |
| 11 | MGNTTSDKGK IPSKVIKSYY GTMDIKKINE GLLDSKILSA | NiVG protein |
| | FNTVIALLGS IVIIVMNIMI IQNYTRSTDN QAVIKDALQG | attachment |
| | IQQQIKGLAD KIGTEIGPKV SLIDTSSTIT IPANIGLLGS | glycoprotein |
| | KISQSTASIN ENVNEKCKFT LPPLKIHECN ISCPNPLPFR | Truncated Δ10 |
| | EYRPQTEGVS NLVGLPNNIC LQKTSNQILK PKLISYTLPV | |
| | VGQSGTCITD PLLAMDEGYF AYSHLERIGS CSRGVSKQRI | |
| | IGVGEVLDRG DEVPSLFMTN VWTPPNPNTV YHCSAVYNNE | |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEILYRA QLASEDTNAQ KTITNCFLLK | |
| | NKIWCISLVE IYDTGDNVIR PKLFAVKIPE QC | |
| 12 | MGKGK IPSKVIKSYY GTMDIKKINE GLLDSKILSA FNTVIALLGS | NiVG protein |
| | IVIIVMNIMI IQNYTRSTDN QAVIKDALQG IQQQIKGLAD | attachment |
| | KIGTEIGPKV SLIDTSSTIT IPANIGLLGS KISQSTASIN | glycoprotein |
| | ENVNEKCKFT LPPLKIHECN ISCPNPLPFR EYRPQTEGVS | Truncated Δ15 |
| | NLVGLPNNIC LQKTSNQILK PKLISYTLPV VGQSGTCITD | |
| | PLLAMDEGYF AYSHLERIGS CSRGVSKQRI IGVGEVLDRG | |
| | DEVPSLFMTN VWTPPNPNTV YHCSAVYNNE FYYVLCAVST | |
| | VGDPILNSTY WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QC | |
| 13 | MGSKVIKSYY GTMDIKKINE GLLDSKILSA FNTVIALLGS | NiVG protein |
| | IVIIVMNIMI IQNYTRSTDN QAVIKDALQG IQQQIKGLAD | attachment |
| | KIGTEIGPKV SLIDTSSTIT IPANIGLLGS KISQSTASIN | glycoprotein |
| | ENVNEKCKFT LPPLKIHECN ISCPNPLPFR EYRPQTEGVS | Truncated Δ20 |
| | NLVGLPNNIC LQKTSNQILK PKLISYTLPV VGQSGTCITD | |
| | PLLAMDEGYF AYSHLERIGS CSRGVSKQRI IGVGEVLDRG | |
| | DEVPSLFMTN VWTPPNPNTV YHCSAVYNNE FYYVLCAVST | |
| | VGDPILNSTY WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QC | |
| 14 | MGSYY GTMDIKKINE GLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated Δ25 |
| | LQKTSNQILK PKLISYTLPV VGQSGTCITD PLLAMDEGYF | |
| | AYSHLERIGS CSRGVSKQRI IGVGEVLDRG DEVPSLFMTN | |
| | VWTPPNPNTV YHCSAVYNNE FYYVLCAVST VGDPILNSTY | |
| | WSGSLMMTRL AVKPKSNGGG YNQHQLALRS IEKGRYDKVM | |
| | PYGPSGIKQG DTLYFPAVGF LVRTEFKYND SNCPITKCQY | |
| | SKPENCRLSM GIRPNSHYIL RSGLLKYNLS DGENPKVVFI | |
| | EISDQRLSIG SPSKIYDSLG QPVFYQASFS WDTMIKFGDV | |
| | LTVNPLVVNW RNNTVISRPG QSQCPRFNTC PEICWEGVYN | |
| | DAFLIDRINW ISAGVFLDSN QTAENPVFTV FKDNEILYRA | |
| | QLASEDTNAQ KTITNCFLLK NKIWCISLVE IYDTGDNVIR | |
| | PKLFAVKIPE QC | |
| 15 | MGTMDIKKINE GLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated Δ30 |
| | LQKTSNQILK PKLISYTLPV VGQSGTCITD PLLAMDEGYF | |
| | AYSHLERIGS CSRGVSKQRI IGVGEVLDRG DEVPSLFMTN | |
| | VWTPPNPNTV YHCSAVYNNE FYYVLCAVST VGDPILNSTY | |
| | WSGSLMMTRL AVKPKSNGGG YNQHQLALRS IEKGRYDKVM | |
| | PYGPSGIKQG DTLYFPAVGF LVRTEFKYND SNCPITKCQY | |
| | SKPENCRLSM GIRPNSHYIL RSGLLKYNLS DGENPKVVFI | |
| | EISDQRLSIG SPSKIYDSLG QPVFYQASFS WDTMIKFGDV | |
| | LTVNPLVVNW RNNTVISRPG QSQCPRFNTC PEICWEGVYN | |
| | DAFLIDRINW ISAGVFLDSN QTAENPVFTV FKDNEILYRA | |
| | QLASEDTNAQ KTITNCFLLK NKIWCISLVE IYDTGDNVIR | |
| | PKLFAVKIPE QC | |
| 16 | MKKINEGLLDSKILSA FNTVIALLGS IVIIVMNIMI IQNYTRSTDN | NiVG protein |
| | QAVIKDALQG IQQQIKGLAD KIGTEIGPKV SLIDTSSTIT | attachment |
| | IPANIGLLGS KISQSTASIN ENVNEKCKFT LPPLKIHECN | glycoprotein |
| | ISCPNPLPFR EYRPQTEGVS NLVGLPNNIC LQKTSNQILK | Truncated and |
| | PKLISYTLPV VGQSGTCITD PLLAMDEGYF AYSHLERIGS | mutated |
| | CSRGVSKQRI IGVGEVLDRG DEVPSLFMTN VWTPPNPNTV | (E501 A, |
| | YHCSAVYNNE FYYVLCAVST VGDPILNSTY WSGSLMMTRL | W504A, Q530A, |
| | AVKPKSNGGG YNQHQLALRS IEKGRYDKVM PYGPSGIKQG | E533A) NiV G |
| | DTLYFPAVGF LVRTEFKYND SNCPITKCQY SKPENCRLSM | protein (Gc Δ |
| | GIRPNSHYIL RSGLLKYNLS DGENPKVVFI EISDQRLSIG | 34) |
| | SPSKIYDSLG QPVFYQASFS WDTMIKFGDV LTVNPLVVNW | |
| | RNNTVISRPG QSQCPRFNTC PAICAEGVYN DAFLIDRINW | |
| | ISAGVFLDSN ATAANPVFTV FKDNEILYRA QLASEDTNAQ | |
| | KTITNCFLLK NKIWCISLVE IYDTGDNVIR PKLFAVKIPE QCT | |
| 17 | MATQEVRLKC LLCGIIVLVL SLEGLGILHY EKLSKIGLVK | Hendra virus F |
| | GITRKYKIKS | protein |
| | NPLTKDIVIK MIPNVSNVSK CTGTVMENYK SRLTGILSPI | Uniprot O89342 |
| | KGAIELYNNN | (with signal |
| | THDLVGDVKL AGVVMAGIAI GIATAAQITA GVALYEAMKN | sequence) |
| | ADNINKLKSS | |
| | IESTNEAVVK LQETAEKTVY VLTALQDYIN TNLVPTIDQI | |
| | SCKQTELALD | |
| | LALSKYLSDL LFVFGPNLQD PVSNSMTIQA ISQAFGGNYE | |
| | TLLRTLGYAT EDFDDLLESD SIAGQIVYVD LSSYYIIVRV | |
| | YFPILTEIQQ AYVQELLPVS | |
| | FNNDNSEWIS IVPNFVLIRN TLISNIEVKY CLITKKSVIC | |
| | NQDYATPMTA | |
| | SVRECLTGST DKCPRELVVS SHVPRFALSG GVLFANCISV | |
| | TCQCQTTGRA ISQSGEQTLL MIDNTTCTTV VLGNIIISLG | |
| | KYLGSINYNS ESIAVGPPVY | |
| | TDKVDISSQI SSMNQSLQQS KDYIKEAQKI LDTVNPSLIS | |
| | MLSMIILYVL | |
| | SIAALCIGLI TFISFVIVEK KRGNYSRLDD RQVRPVSNGD LYYIGT | |
| 18 | MMADSKLVSL NNNLSGKIKD QGKVIKNYYG TMDIKKINDG | Hendra virus G |
| | LLDSKILGAF | protein Uniprot |
| | NTVIALLGSI IIIVMNIMII QNYTRTTDNQ ALIKESLQSV | O89343 |
| | QQQIKALTDK IGTEIGPKVS LIDTSSTITI PANIGLLGSK | |
| | ISQSTSSINE NVNDKCKFTL | |
| | PPLKIHECNI SCPNPLPFRE YRPISQGVSD LVGLPNQICL | |
| | QKTTSTILKP RLISYTLPIN TREGVCITDP LLAVDNGFFA | |
| | YSHLEKIGSC TRGIAKQRII GVGEVLDRGD KVPSMFMTNV | |
| | WTPPNPSTIH HCSSTYHEDF YYTLCAVSHV | |
| | GDPILNSTSW TESLSLIRLA VRPKSDSGDY NQKYIAITKV | |
| | ERGKYDKVMP | |
| | YGPSGIKQGD TLYFPAVGFL PRTEFQYNDS NCPIIHCKYS | |
| | KAENCRLSMG | |
| | VNSKSHYILR SGLLKYNLSL GGDIILQFIE IADNRLTIGS | |
| | PSKIYNSLGQ PVFYQASYSW DTMIKLGDVD TVDPLRVQWR | |
| | NNSVISRPGQ SQCPRFNVCP | |
| | EVCWEGTYND AFLIDRLNWV SAGVYLNSNQ TAENPVFAVF | |
| | KDNEILYQVP LAEDDTNAQK TITDCFLLEN VIWCISLVEI | |
| | YDTGDSVIRP KLFAVKIPAQ CSES | |
| 19 | MVVILDKRCY CNLLILILMI SECSVGILHY EKLSKIGLVK | Nipah virus |
| | GVTRKYKIKS NPLTKDIVIK MIPNVSNMSQ CTGSVMENYK | NiV-F F0 T234 |
| | TRLNGILTPI KGALEIYKNN THDLVGDVRL AGVIMAGVAI | truncation (aa |
| | GIATAAQITA GVALYEAMKN ADNINKLKSS IESTNEAVVK | 525-544)(with |
| | LQETAEKTVY VLTALQDYIN TNLVPTIDKI SCKQTELSLD | signal sequence) |
| | LALSKYLSDL LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNTGT | |
| 20 | MVVILDKRCY CNLLILILMI SECSVGILHY EKLSKIGLVK | Nipah virus |
| | GVTRKYKIKS NPLTKDIVIK MIPNVSNMSQ CTGSVMENYK | NiV-F F0 T234 |
| | TRLNGILTPI KGALEIYKNQ THDLVGDVRL AGVIMAGVAI | truncation (aa |
| | GIATAAQITA GVALYEAMKN ADNINKLKSS IESTNEAVVK | 525-544) AND |
| | LQETAEKTVY VLTALQDYIN TNLVPTIDKI SCKQTELSLD | mutation on N- |
| | LALSKYLSDL LFVFGPNLQD PVSNSMTIQA ISQAFGGNYE | linked |
| | TLLRTLGYAT EDFDDLLESD SITGQIIYVD LSSYYIIVRV | glycosylation |
| | YFPILTEIQQ AYIQELLPVS FNNDNSEWIS IVPNFILVRN | site (with signal |
| | TLISNIEIGF CLITKRSVIC NQDYATPMTN NMRECLTGST | sequence) |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNTGT | |
| 21 | MVVILDKRCY CNLLILILMI SECSVGILHY EKLSKIGLVK | Truncated NiV |
| | GVTRKYKIKS NPLTKDIVIK MIPNVSNMSQ CTGSVMENYK | fusion |
| | TRLNGILTPI KGALEIYKNN THDLVGDVRL AGVIMAGVAI | glycoprotein |
| | GIATAAQITA GVALYEAMKN ADNINKLKSS IESTNEAVVK | (FcDelta22) at |
| | LQETAEKTVY VLTALQDYIN TNLVPTIDKI SCKQTELSLD | cytoplasmic tail |
| | LALSKYLSDL LFVFGPNLQD PVSNSMTIQA ISQAFGGNYE | (with signal |
| | TLLRTLGYAT EDFDDLLESD SITGQIIYVD LSSYYIIVRV | sequence) |
| | YFPILTEIQQ AYIQELLPVS FNNDNSEWIS IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNT | |
| 22 | MKKINEGLLDSKILSA FNTVIALLGS IVIIVMNIMI IQNYTRSTDN | NiVG protein |
| | QAVIKDALQG IQQQIKGLAD KIGTEIGPKV SLIDTSSTIT | attachment |
| | IPANIGLLGS KISQSTASIN ENVNEKCKFT LPPLKIHECN | glycoprotein |
| | ISCPNPLPFR EYRPQTEGVS NLVGLPNNIC LQKTSNQILK | Truncated (Gc Δ |
| | PKLISYTLPV VGQSGTCITD PLLAMDEGYF AYSHLERIGS | 34) |
| | CSRGVSKQRI IGVGEVLDRG DEVPSLFMTN VWTPPNPNTV | |
| | YHCSAVYNNE FYYVLCAVST VGDPILNSTY WSGSLMMTRL | |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRFNTC PEICWEGVYN DAFLIDRINW | |
| | ISAGVFLDSN QTAENPVFTV FKDNEILYRA QLASEDTNAQ | |
| | KTITNCFLLK NKIWCISLVE IYDTGDNVIR PKLFAVKIPE QCT | |
| 23 | ILHY EKLSKIGLVK GVTRKYKIKS NPLTKDIVIK MIPNVSNMSQ | Truncated |
| | CTGSVMENYK TRLNGILTPI KGALEIYKNN THDLVGDVRL | mature NiV |
| | AGVIMAGVAI GIATAAQITA GVALYEAMKN ADNINKLKSS | fusion |
| | IESTNEAVVK LQETAEKTVY VLTALQDYIN TNLVPTIDKI | glycoprotein |
| | SCKQTELSLD LALSKYLSDL LFVFGPNLQD PVSNSMTIQA | (FcDelta22) at |
| | ISQAFGGNYE TLLRTLGYAT EDFDDLLESD SITGQIIYVD | cytoplasmic tail |
| | LSSYYIIVRV YFPILTEIQQ AYIQELLPVS FNNDNSEWIS | |
| | IVPNFILVRN TLISNIEIGF CLITKRSVIC NQDYATPMTN | |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI | |
| | TFISFIIVEK KRNT | |
| 24 | MSNKRTTVLIIISYTLFYLNNAAIVGFDFDKLNKIGVVQGRVLNYKIKGDP | gb: JQ001776: 61 |
| | MTKDLVLKFIPNIVNITECVREPLSRYNETVRRLLLPIHNMLGLYLNNTNA | 29- |
| | KMTGLMIAGVIMGGIAIGIATAAQITAGFALYEAKKNTENIQKLTDSIMKT | 8166\|Organism: |
| | QDSIDKLTDSVGTSILILNKLQTYINNQLVPNLELLSCRQNKIEFDLMLTK | Cedar |
| | YLVDLMTVIGPNINNPVNKDMTIQSLSLLFDGNYDIMMSELGYTPQDFLDL | virus\|Strain |
| | IESKSITGQIIYVDMENLYVVIRTYLPTLIEVPDAQIYEFNKITMSSNGGE | Name: CG1a\|Prot |
| | YLSTIPNFILIRGNYMSNIDVATCYMTKASVICNQDYSLPMSQNLRSCYQG | ein Name: fusion |
| | ETEYCPVEAVIASHSPRFALTNGVIFANCINTICRCQDNGKTITQNINQFV | glycoprotein\|Gen |
| | SMIDNSTCNDVMVDKFTIKVGKYMGRKDINNINIQIGPQIIIDKVDLSNEI | e Symbol: F |
| | NKMNQSLKDSIFYLREAKRILDSVNISLISPSVQLFLIIISVLSFIILLII | (with signal |
| | IVYLYCKSKHSYKYNKFIDDPDYYNDYKRERINGKASKSNNIYYVGD | sequence) |
| 25 | MALNKNMFSSLFLGYLLVYATTVQSSIHYDSLSKVGVIKGLTYNYKIKGSP | gb: NC_025352: 5 |
| | STKLMVVKLIPNIDSVKNCTQKQYDEYKNLVRKALEPVKMAIDTMLNNVKS | 950- |
| | GNNKYRFAGAIMAGVALGVATAATVTAGIALHRSNENAQAIANMKSAIQNT | 8712\|Organism: |
| | NEAVKQLQLANKQTLAVIDTIRGEINNNIIPVINQLSCDTIGLSVGIRLTQ | Mojiang |
| | YYSEIITAFGPALQNPVNTRITIQAISSVFNGNFDELLKIMGYTSGDLYEI | virus\|Strain |
| | LHSELIRGNIIDVDVDAGYIALEIEFPNLTLVPNAVVQELMPISYNIDGDE | Name: Tongguan |
| | WVTLVPRFVLTRTTLLSNIDTSRCTITDSSVICDNDYALPMSHELIGCLQG | 1\|Protein |
| | DISKCAREKVVSSYVPKFALSDGLVYANCLNTICRCMDTDTPISQSLGATV | Name: fusion |
| | SLLDNKRCSVYQVGDVLISVGSYLGDGEYNADNVELGPPIVIDKIDIGNQL | protein\|lGene |
| | AGINQTLQEAEDYIEKSEEFLKGVNPSIITLGSMVVLYIFMILIAIVSVIA | Symbol: F (with |
| | LVLSIKLTVKGNVVRQQFTYTQHVPSMENINYVSH | signal sequence) |
| 26 | MKKKTDNPTISKRGHNHSRGIKSRALLRETDNYSNGLIVENLVRNCHHPSK | gb: NC_025256: 6 |
| | NNLNYTKTQKRDSTIPYRVEERKGHYPKIKHLIDKSYKHIKRGKRRNGHNG | 865- |
| | NIITIILLLILILKTQMSEGAIHYETLSKIGLIKGITREYKVKGTPSSKDI | 8853\|Organism: |
| | VIKLIPNVTGLNKCTNISMENYKEQLDKILIPINNIIELYANSTKSAPGNA | Bat |
| | RFAGVIIAGVALGVAAAAQITAGIALHEARQNAERINLLKDSISATNNAVA | Paramyxovirus |
| | ELQEATGGIVNVITGMQDYINTNLVPQIDKLQCSQIKTALDISLSQYYSEI | Eid_hel/GH- |
| | LTVFGPNLQNPVTTSMSIQAISQSFGGNIDLLLNLLGYTANDLLDLLESKS | M74a/GHA/200 |
| | ITGQITYINLEHYFMVIRVYYPIMTTISNAYVQELIKISFNVDGSEWVSLV | 9\|Strain |
| | PSYILIRNSYLSNIDISECLITKNSVICRHDFAMPMSYTLKECLTGDTEKC | Name: BatPV/Ei |
| | PREAVVTSYVPRFAISGGVIYANCLSTTCQCYQTGKVIAQDGSQTLMMIDN | d_hel/GH- |
| | QTCSIVRIEEILISTGKYLGSQEYNTMHVSVGNPVFTDKLDITSQISNINQ | M74a/GHA/200 |
| | SIEQSKFYLDKSKAILDKINLNLIGSVPISILFIIAILSLILSIITFVIVM | 9\|Protein |
| | IIVRRYNKYTPLINSDPSSRRSTIQDVYIIPNPGEHSIRSAARSIDRDRD | Name: fusion |
| | | protein\|Gene |
| | | Symbol: F (with |
| | | signal sequence) |
| 27 | (GGGGGS)n wherein n is 1 to 6 | Peptide Linker |
| 28 | MPAENKKVRFENTTSDKGKIPSKVIKSYYGTMDIKKINEGLLDSKILSAFN | gb: AF212302\|Or |
| | TVIALLGSIVIIVMNIMIIQNYTRSTDNQAVIKDALQGIQQQIKGLADKIG | ganism: Nipah |
| | TEIGPKVSLIDTSSTITIPANIGLLGSKISQSTASINENVNEKCKFTLPPL | virus\|Strain |
| | KIHECNISCPNPLPFREYRPQTEGVSNLVGLPNNICLQKTSNQILKPKLIS | Name: UNKNO |
| | YTLPVVGQSGTCITDPLLAMDEGYFAYSHLERIGSCSRGVSKQRIIGVGEV | WN- |
| | LDRGDEVPSLFMTNVWTPPNPNTVYHCSAVYNNEFYYVLCAVSTVGDPILN | AF212302\|Protei |
| | STYWSGSLMMTRLAVKPKSNGGGYNQHQLALRSIEKGRYDKVMPYGPSGIK | n |
| | QGDTLYFPAVGFLVRTEFKYNDSNCPITKCQYSKPENCRLSMGIRPNSHYI | Name: attachmen |
| | LRSGLLKYNLSDGENPKVVFIEISDQRLSIGSPSKIYDSLGQPVFYQASFS | t |
| | WDTMIKFGDVLTVNPLVVNWRNNTVISRPGQSQCPRFNTCPEICWEGVYND | glycoprotein\|Gen |
| | AFLIDRINWISAGVFLDSNQTAENPVFTVFKDNEILYRAQLASEDTNAQKT | e Symbol: G |
| | ITNCFLLKNKIWCISLVEIYDTGDNVIRPKLFAVKIPEQCT | (Uniprot |
| | | Q9IH62) |
| 29 | MLSQLQKNYLDNSNQQGDKMNNPDKKLSVNFNPLELDKGQKDLNKSYYVKN | gb: JQ001776: 81 |
| | KNYNVSNLLNESLHDIKFCIYCIFSLLIIITIINIITISIVITRLKVHEEN | 70- |
| | NGMESPNLQSIQDSLSSLTNMINTEITPRIGILVTATSVILSSSINYVGTK | 10275\|Organism: |
| | TNQLVNELKDYITKSCGFKVPELKLHECNISCADPKISKSAMYSTNAYAEL | Cedar |
| | AGPPKIFCKSVSKDPDFRLKQIDYVIPVQQDRSICMNNPLLDISDGFFTYI | virus\|Strain |
| | HYEGINSCKKSDSFKVLLSHGEIVDRGDYRPSLYLLSSHYHPYSMQVINCV | Name: CG1a\|Prot |
| | PVTCNQSSFVFCHISNNTKTLDNSDYSSDEYYITYFNGIDRPKTKKIPINN | ein |
| | MTADNRYIHFTFSGGGGVCLGEEFIIPVTTVINTDVFTHDYCESFNCSVQT | Name: attachmen |
| | GKSLKEICSESLRSPTNSSRYNLNGIMIISQNNMTDFKIQLNGITYNKLSF | t |
| | GSPGRLSKTLGQVLYYQSSMSWDTYLKAGFVEKWKPFTPNWMNNTVISRPN | glycoprotein\|Gen |
| | QGNCPRYHKCPEICYGGTYNDIAPLDLGKDMYVSVILDSDQLAENPEITVF | e Symbol: G |
| | NSTTILYKERVSKDELNTRSTTTSCFLFLDEPWCISVLETNRFNGKSIRPE | |
| | IYSYKIPKYC | |
| 30 | MPQKTVEFINMNSPLERGVSTLSDKKTLNQSKITKQGYFGLGSHSERNWKK | gb: NC_025256: 9 |
| | QKNQNDHYMTVSTMILEILVVLGIMFNLIVLTMVYYQNDNINQRMAELTSN | 117- |
| | ITVLNLNLNQLINKIQREIIPRITLIDTATTITIPSAITYILATLTTRISE | 11015\|Organism: |
| | LLPSINQKCEFKTPTLVLNDCRINCTPPLNPSDGVKMSSLATNLVAHGPSP | Bat |
| | CRNFSSVPTIYYYRIPGLYNRTALDERCILNPRLTISSTKFAYVHSEYDKN | Paramyxovirus |
| | CTRGFKYYELMTFGEILEGPEKEPRMFSRSFYSPTNAVNYHSCTPIVTVNE | Eid_hel/GH- |
| | GYFLCLECTSSDPLYKANLSNSTFHLVILRHNKDEKIVSMPSFNLSTDQEY | M74a/GHA/200 |
| | VQIIPAEGGGTAESGNLYFPCIGRLLHKRVTHPLCKKSNCSRTDDESCLKS | 9\|Strain |
| | YYNQGSPQHQVVNCLIRIRNAQRDNPTWDVITVDLTNTYPGSRSRIFGSFS | Name: BatPV/Ei |
| | KPMLYQSSVSWHTLLQVAEITDLDKYQLDWLDTPYISRPGGSECPFGNYCP | d_hel/GH- |
| | TVCWEGTYNDVYSLTPNNDLFVTVYLKSEQVAENPYFAIFSRDQILKEFPL | M74a/GHA/200 |
| | DAWISSARTTTISCFMFNNEIWCIAALEITRLNDDIIRPIYYSFWLPTDCR | 9\|Protein |
| | TPYPHTGKMTRVPLRSTYNY | Name: glycoprote |
| | | in\|Gene |
| | | Symbol: G |
| 31 | MATNRDNTITSAEVSQEDKVKKYYGVETAEKVADSISGNKVFILMNTLLIL | gb: NC_025352: 8 |
| | TGAIITITLNITNLTAAKSQQNMLKIIQDDVNAKLEMFVNLDQLVKGEIKP | 716- |
| | KVSLINTAVSVSIPGQISNLQTKFLQKYVYLEESITKQCTCNPLSGIFPTS | 11257}Organtsm: |
| | GPTYPPTDKPDDDTTDDDKVDTTIKPIEYPKPDGCNRTGDHFTMEPGANFY | Mojiang |
| | TVPNLGPASSNSDECYTNPSFSIGSSIYMFSQEIRKTDCTAGEILSIQIVL | virus\|Strain |
| | GRIVDKGQQGPQASPLLVWAVPNPKIINSCAVAAGDEMGWVLCSVTLTAAS | Name: Tongguan |
| | GEPIPHMFDGFWLYKLEPDTEVVSYRITGYAYLLDKQYDSVFIGKGGGIQK | 1\|Protein |
| | GNDLYFQMYGLSRNRQSFKALCEHGSCLGTGGGGYQVLCDRAVMSFGSEES | Name: attachmen |
| | LITNAYLKVNDLASGKPVIIGQTFPPSDSYKGSNGRMYTIGDKYGLYLAPS | t |
| | SWNRYLRFGITPDISVRSTTWLKSQDPIMKILSTCTNTDRDMCPEICNTRG | glycoprotein\|Gen |
| | YQDIFPLSEDSEYYTYIGITPNNGGTKNFVAVRDSDGHIASIDILQNYYSI | e Symbol: G |
| | TSATISCFMYKDEIWCIAITEGKKQKDNPQRIYAHSYKIRQMCYNMKSATV | |
| | TVGNAKNITIRRY | |
| 32 | FNTVIALLGS IVIIVMNIMI IQNYTRSTDN QAVIKDALQG | NivG protein |
| | IQQQIKGLAD KIGTEIGPKV SLIDTSSTIT IPANIGLLGS | attachment |
| | KISQSTASIN ENVNEKCKFT LPPLKIHECN ISCPNPLPFR | glycoprotein |
| | EYRPQTEGVS NLVGLPNNIC LQKTSNQILK PKLISYTLPV | Without |
| | VGQSGTCITD PLLAMDEGYF AYSHLERIGS CSRGVSKQRI | cytoplasmic tail |
| | IGVGEVLDRG DEVPSLFMTN VWTPPNPNTV YHCSAVYNNE | Uniprot Q9IH62 |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEILYRA QLASEDTNAQ KTITNCFLLK | |
| | NKIWCISLVE IYDTGDNVIR PKLFAVKIPE QC | |
| 33 | FNTVIALLGSI IIIVMNIMII QNYTRTTDNQ ALIKESLQSV | Hendra virus G |
| | QQQIKALTDK | protein Uniprot |
| | IGTEIGPKVS LIDTSSTITI PANIGLLGSK ISQSTSSINE | O89343 |
| | NVNDKCKFTL | Without |
| | PPLKIHECNI SCPNPLPFRE YRPISQGVSD LVGLPNQICL | cytoplasmic tail |
| | QKTTSTILKP | |
| | RLISYTLPIN TREGVCITDP LLAVDNGFFA YSHLEKIGSC | |
| | TRGIAKQRII | |
| | GVGEVLDRGD KVPSMFMTNV WTPPNPSTIH HCSSTYHEDF | |
| | YYTLCAVSHV | |
| | GDPILNSTSW TESLSLIRLA VRPKSDSGDY NQKYIAITKV | |
| | ERGKYDKVMP | |
| | YGPSGIKQGD TLYFPAVGFL PRTEFQYNDS NCPIIHCKYS | |
| | KAENCRLSMG | |
| | VNSKSHYILR SGLLKYNLSL GGDIILQFIE IADNRLTIGS | |
| | PSKIYNSLGQ | |
| | PVFYQASYSW DTMIKLGDVD TVDPLRVQWR NNSVISRPGQ | |
| | SQCPRFNVCP EVCWEGTYND AFLIDRLNWV SAGVYLNSNQ | |
| | TAENPVFAVF KDNEILYQVP LAEDDTNAQK TITDCFLLEN | |
| | VIWCISLVEI YDTGDSVIRP KLFAVKIPAQ CSES | |
| 34 | MVVILDKRCY CNLLILILMI SECSVG | signal sequence |
| 35 | MKVR FENTTSDKGK IPSKVIKSYY GTMDIKKINE GLLDSKILSA | NiVG protein |
| | FNTVIALLGS IVIIVMNIMI IQNYTRSTDN QAVIKDALQG | attachment |
| | IQQQIKGLAD KIGTEIGPKV SLIDTSSTIT IPANIGLLGS | glycoprotein |
| | KISQSTASIN ENVNEKCKFT LPPLKIHECN ISCPNPLPFR | Truncated 45 |
| | EYRPQTEGVS NLVGLPNNIC LQKTSNQILK PKLISYTLPV | |
| | VGQSGTCITD PLLAMDEGYF AYSHLERIGS CSRGVSKQRI | |
| | IGVGEVLDRG DEVPSLFMTN VWTPPNPNTV YHCSAVYNNE | |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEILYRA QLASEDTNAQ KTITNCFLLK | |
| | NKIWCISLVE IYDTGDNVIR PKLFAVKIPE QCT | |
| 36 | MNTTSDKGK IPSKVIKSYY GTMDIKKINE GLLDSKILSA | NiVG protein |
| | FNTVIALLGS IVIIVMNIMI IQNYTRSTDN QAVIKDALQG | attachment |
| | IQQQIKGLAD KIGTEIGPKV SLIDTSSTIT IPANIGLLGS | glycoprotein |
| | KISQSTASIN ENVNEKCKFT LPPLKIHECN ISCPNPLPFR | Truncated Δ10 |
| | EYRPQTEGVS NLVGLPNNIC LQKTSNQILK PKLISYTLPV | |
| | VGQSGTCITD PLLAMDEGYF AYSHLERIGS CSRGVSKQRI | |
| | IGVGEVLDRG DEVPSLFMTN VWTPPNPNTV YHCSAVYNNE | |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEILYRA QLASEDTNAQ KTITNCFLLK | |
| | NKIWCISLVE IYDTGDNVIR PKLFAVKIPE QCT | |
| 37 | MKGK IPSKVIKSYY GTMDIKKINE GLLDSKILSA FNTVIALLGS | NiVG protein |
| | IVIIVMNIMI IQNYTRSTDN QAVIKDALQG IQQQIKGLAD | attachment |
| | KIGTEIGPKV SLIDTSSTIT IPANIGLLGS KISQSTASIN | glycoprotein |
| | ENVNEKCKFT LPPLKIHECN ISCPNPLPFR EYRPQTEGVS | Truncated Δ15 |
| | NLVGLPNNIC LQKTSNQILK PKLISYTLPV VGQSGTCITD | |
| | PLLAMDEGYF AYSHLERIGS CSRGVSKQRI IGVGEVLDRG | |
| | DEVPSLFMTN VWTPPNPNTV YHCSAVYNNE FYYVLCAVST | |
| | VGDPILNSTY WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QCT | |
| 38 | MSKVIKSYY GTMDIKKINE GLLDSKILSA FNTVIALLGS | NiVG protein |
| | IVIIVMNIMI IQNYTRSTDN QAVIKDALQG IQQQIKGLAD | attachment |
| | KIGTEIGPKV SLIDTSSTIT IPANIGLLGS KISQSTASIN | glycoprotein |
| | ENVNEKCKFT LPPLKIHECN ISCPNPLPFR EYRPQTEGVS | Truncated Δ20 |
| | NLVGLPNNIC LQKTSNQILK PKLISYTLPV VGQSGTCITD | |
| | PLLAMDEGYF AYSHLERIGS CSRGVSKQRI IGVGEVLDRG | |
| | DEVPSLFMTN VWTPPNPNTV YHCSAVYNNE FYYVLCAVST | |
| | VGDPILNSTY WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QCT | |
| 39 | MSYY GTMDIKKINE GLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated Δ25 |
| | LQKTSNQILK PKLISYTLPV VGQSGTCITD PLLAMDEGYF | |
| | AYSHLERIGS CSRGVSKQRI IGVGEVLDRG DEVPSLFMTN | |
| | VWTPPNPNTV YHCSAVYNNE FYYVLCAVST VGDPILNSTY | |
| | WSGSLMMTRL AVKPKSNGGG YNQHQLALRS IEKGRYDKVM | |
| | PYGPSGIKQG DTLYFPAVGF LVRTEFKYND SNCPITKCQY | |
| | SKPENCRLSM GIRPNSHYIL RSGLLKYNLS DGENPKVVFI | |
| | EISDQRLSIG SPSKIYDSLG QPVFYQASFS WDTMIKFGDV | |
| | LTVNPLVVNW RNNTVISRPG QSQCPRFNTC PEICWEGVYN | |
| | DAFLIDRINW ISAGVFLDSN QTAENPVFTV FKDNEILYRA | |
| | QLASEDTNAQ KTITNCFLLK NKIWCISLVE IYDTGDNVIR | |
| | PKLFAVKIPE QCT | |
| 40 | MTMDIKKINE GLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated Δ30 |
| | LQKTSNQILK PKLISYTLPV VGQSGTCITD PLLAMDEGYF | |
| | AYSHLERIGS CSRGVSKQRI IGVGEVLDRG DEVPSLFMTN | |
| | VWTPPNPNTV YHCSAVYNNE FYYVLCAVST VGDPILNSTY | |
| | WSGSLMMTRL AVKPKSNGGG YNQHQLALRS IEKGRYDKVM | |
| | PYGPSGIKQG DTLYFPAVGF LVRTEFKYND SNCPITKCQY | |
| | SKPENCRLSM GIRPNSHYIL RSGLLKYNLS DGENPKVVFI | |
| | EISDQRLSIG SPSKIYDSLG QPVFYQASFS WDTMIKFGDV | |
| | LTVNPLVVNW RNNTVISRPG QSQCPRFNTC PEICWEGVYN | |
| | DAFLIDRINW ISAGVFLDSN QTAENPVFTV FKDNEILYRA | |
| | QLASEDTNAQ KTITNCFLLK NKIWCISLVE IYDTGDNVIR | |
| | PKLFAVKIPE QCT | |
| 41 | GGGGGS | Peptide linker |
| 42 | (GGGGS)n wherein n is 1 to 10 | Peptide linker |
| 43 | GGGGS | Peptide linker |
| 44 | PAENKKVR FENTTSDKGK IPSKVIKSYY GTMDIKKINE | NiVG protein |
| | GLLDSKILSA FNTVIALLGS IVIIVMNIMI IQNYTRSTDN | attachment |
| | QAVIKDALQG IQQQIKGLAD KIGTEIGPKV SLIDTSSTIT | glycoprotein |
| | IPANIGLLGS KISQSTASIN ENVNEKCKFT LPPLKIHECN | (602 aa) |
| | ISCPNPLPFR EYRPQTEGVS NLVGLPNNIC LQKTSNQILK | Without N- |
| | PKLISYTLPV VGQSGTCITD PLLAMDEGYF AYSHLERIGS | terminal |
| | CSRGVSKQRI IGVGEVLDRG DEVPSLFMTN VWTPPNPNTV | methionine |
| | YHCSAVYNNE FYYVLCAVST VGDPILNSTY WSGSLMMTRL | |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRFNTC PEICWEGVYN DAFLIDRINW | |
| | ISAGVFLDSN QTAENPVFTV FKDNEILYRA QLASEDTNAQ | |
| | KTITNCFLLK NKIWCISLVE IYDTGDNVIR PKLFAVKIPE QC | |
| 45 | KVR FENTTSDKGK IPSKVIKSYY GTMDIKKINE GLLDSKILSA | NiVG protein |
| | FNTVIALLGS IVIIVMNIMI IQNYTRSTDN QAVIKDALQG | attachment |
| | IQQQIKGLAD KIGTEIGPKV SLIDTSSTIT IPANIGLLGS | glycoprotein |
| | KISQSTASIN ENVNEKCKFT LPPLKIHECN ISCPNPLPFR | Truncated Δ5 |
| | EYRPQTEGVS NLVGLPNNIC LQKTSNQILK PKLISYTLPV | Without N- |
| | VGQSGTCITD PLLAMDEGYF AYSHLERIGS CSRGVSKQRI | terminal |
| | IGVGEVLDRG DEVPSLFMTN VWTPPNPNTV YHCSAVYNNE | methionine |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEILYRA QLASEDTNAQ KTITNCFLLK | |
| | NKIWCISLVE IYDTGDNVIR PKLFAVKIPE QC | |
| 46 | NTTSDKGK IPSKVIKSYY GTMDIKKINE GLLDSKILSA | NiVG protein |
| | FNTVIALLGS IVIIVMNIMI IQNYTRSTDN QAVIKDALQG | attachment |
| | IQQQIKGLAD KIGTEIGPKV SLIDTSSTIT IPANIGLLGS | glycoprotein |
| | KISQSTASIN ENVNEKCKFT LPPLKIHECN ISCPNPLPFR | Truncated Δ10 |
| | EYRPQTEGVS NLVGLPNNIC LQKTSNQILK PKLISYTLPV | Without N- |
| | VGQSGTCITD PLLAMDEGYF AYSHLERIGS CSRGVSKQRI | terminal |
| | IGVGEVLDRG DEVPSLFMTN VWTPPNPNTV YHCSAVYNNE | methionine |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEILYRA QLASEDTNAQ KTITNCFLLK | |
| | NKIWCISLVE IYDTGDNVIR PKLFAVKIPE QC | |
| 47 | KGK IPSKVIKSYY GTMDIKKINE GLLDSKILSA FNTVIALLGS | NiVG protein |
| | IVIIVMNIMI IQNYTRSTDN QAVIKDALQG IQQQIKGLAD | attachment |
| | KIGTEIGPKV SLIDTSSTIT IPANIGLLGS KISQSTASIN | glycoprotein |
| | ENVNEKCKFT LPPLKIHECN ISCPNPLPFR EYRPQTEGVS | Truncated 4 5 |
| | NLVGLPNNIC LQKTSNQILK PKLISYTLPV VGQSGTCITD | Without N- |
| | PLLAMDEGYF AYSHLERIGS CSRGVSKQRI IGVGEVLDRG | terminal |
| | DEVPSLFMTN VWTPPNPNTV YHCSAVYNNE FYYVLCAVST | methionine |
| | VGDPILNSTY WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QC | |
| 48 | SKVIKSYY GTMDIKKINE GLLDSKILSA FNTVIALLGS | NiVG protein |
| | IVIIVMNIMI IQNYTRSTDN QAVIKDALQG IQQQIKGLAD | attachment |
| | KIGTEIGPKV SLIDTSSTIT IPANIGLLGS KISQSTASIN | glycoprotein |
| | ENVNEKCKFT LPPLKIHECN ISCPNPLPFR EYRPQTEGVS | Truncated Δ20 |
| | NLVGLPNNIC LQKTSNQILK PKLISYTLPV VGQSGTCITD | Without N- |
| | PLLAMDEGYF AYSHLERIGS CSRGVSKQRI IGVGEVLDRG | terminal |
| | DEVPSLFMTN VWTPPNPNTV YHCSAVYNNE FYYVLCAVST | methionine |
| | VGDPILNSTY WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QC | |
| 49 | SYY GTMDIKKINE GLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated Δ25 |
| | LQKTSNQILK PKLISYTLPV VGQSGTCITD PLLAMDEGYF | Without N- |
| | AYSHLERIGS CSRGVSKQRI IGVGEVLDRG DEVPSLFMTN | terminal |
| | VWTPPNPNTV YHCSAVYNNE FYYVLCAVST VGDPILNSTY | methionine |
| | WSGSLMMTRL AVKPKSNGGG YNQHQLALRS IEKGRYDKVM | |
| | PYGPSGIKQG DTLYFPAVGF LVRTEFKYND SNCPITKCQY | |
| | SKPENCRLSM GIRPNSHYIL RSGLLKYNLS DGENPKVVFI | |
| | EISDQRLSIG SPSKIYDSLG QPVFYQASFS WDTMIKFGDV | |
| | LTVNPLVVNW RNNTVISRPG QSQCPRFNTC PEICWEGVYN | |
| | DAFLIDRINW ISAGVFLDSN QTAENPVFTV FKDNEILYRA | |
| | QLASEDTNAQ KTITNCFLLK NKIWCISLVE IYDTGDNVIR | |
| | PKLFAVKIPE QC | |
| 50 | TMDIKKINE GLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated Δ30 |
| | LQKTSNQILK PKLISYTLPV VGQSGTCITD PLLAMDEGYF | Without N- |
| | AYSHLERIGS CSRGVSKQRI IGVGEVLDRG DEVPSLFMTN | terminal |
| | VWTPPNPNTV YHCSAVYNNE FYYVLCAVST VGDPILNSTY | methionine |
| | WSGSLMMTRL AVKPKSNGGG YNQHQLALRS IEKGRYDKVM | |
| | PYGPSGIKQG DTLYFPAVGF LVRTEFKYND SNCPITKCQY | |
| | SKPENCRLSM GIRPNSHYIL RSGLLKYNLS DGENPKVVFI | |
| | EISDQRLSIG SPSKIYDSLG QPVFYQASFS WDTMIKFGDV | |
| | LTVNPLVVNW RNNTVISRPG QSQCPRFNTC PEICWEGVYN | |
| | DAFLIDRINW ISAGVFLDSN QTAENPVFTV FKDNEILYRA | |
| | QLASEDTNAQ KTITNCFLLK NKIWCISLVE IYDTGDNVIR | |
| | PKLFAVKIPE QC | |
| 51 | KKINEGLLDSKILSA FNTVIALLGS IVIIVMNIMI IQNYTRSTDN | NiVG protein |
| | QAVIKDALQG IQQQIKGLAD KIGTEIGPKV SLIDTSSTIT | attachment |
| | IPANIGLLGS KISQSTASIN ENVNEKCKFT LPPLKIHECN | glycoprotein |
| | ISCPNPLPFR EYRPQTEGVS NLVGLPNNIC LQKTSNQILK | Truncated and |
| | PKLISYTLPV VGQSGTCITD PLLAMDEGYF AYSHLERIGS | mutated |
| | CSRGVSKQRI IGVGEVLDRG DEVPSLFMTN VWTPPNPNTV | (E501 A, |
| | YHCSAVYNNE FYYVLCAVST VGDPILNSTY WSGSLMMTRL | W504A, Q530A, |
| | AVKPKSNGGG YNQHQLALRS IEKGRYDKVM PYGPSGIKQG | E533A) NiV G |
| | DTLYFPAVGF LVRTEFKYND SNCPITKCQY SKPENCRLSM | protein (Gc Δ |
| | GIRPNSHYIL RSGLLKYNLS DGENPKVVFI EISDQRLSIG | 34) Without N- |
| | SPSKIYDSLG QPVFYQASFS WDTMIKFGDV LTVNPLVVNW | terminal |
| | RNNTVISRPG QSQCPRFNTC PAICAEGVYN DAFLIDRINW | methionine |
| | ISAGVFLDSN ATAANPVFTV FKDNEILYRA QLASEDTNAQ | |
| | KTITNCFLLK NKIWCISLVE IYDTGDNVIR PKLFAVKIPE QCT | |
| 52 | MADSKLVSL NNNLSGKIKD QGKVIKNYYG TMDIKKINDG | Hendra virus G |
| | LLDSKILGAF | protein Uniprot |
| | NTVIALLGSI IIIVMNIMII QNYTRTTDNQ ALIKESLQSV | O89343 Without |
| | QQQIKALTDK IGTEIGPKVS LIDTSSTITI PANIGLLGSK | N-terminal |
| | ISQSTSSINE NVNDKCKFTL | methionine |
| | PPLKIHECNI SCPNPLPFRE YRPISQGVSD LVGLPNQICL | |
| | QKTTSTILKP RLISYTLPIN TREGVCITDP LLAVDNGFFA | |
| | YSHLEKIGSC TRGIAKQRII GVGEVLDRGD KVPSMFMTNV | |
| | WTPPNPSTIH HCSSTYHEDF YYTLCAVSHV | |
| | GDPILNSTSW TESLSLIRLA VRPKSDSGDY NQKYIAITKV | |
| | ERGKYDKVMP | |
| | YGPSGIKQGD TLYFPAVGFL PRTEFQYNDS NCPIIHCKYS | |
| | KAENCRLSMG | |
| | VNSKSHYILR SGLLKYNLSL GGDIILQFIE IADNRLTIGS | |
| | PSKIYNSLGQ PVFYQASYSW DTMIKLGDVD TVDPLRVQWR | |
| | NNSVISRPGQ SQCPRFNVCP | |
| | EVCWEGTYND AFLIDRLNWV SAGVYLNSNQ TAENPVFAVF | |
| | KDNEILYQVP LAEDDTNAQK TITDCFLLEN VIWCISLVEI | |
| | YDTGDSVIRP KLFAVKIPAQ CSES | |
| 53 | KKINEGLLDSKILSA FNTVIALLGS IVIIVMNIMI IQNYTRSTDN | NiVG protein |
| | QAVIKDALQG IQQQIKGLAD KIGTEIGPKV SLIDTSSTIT | attachment |
| | IPANIGLLGS KISQSTASIN ENVNEKCKFT LPPLKIHECN | glycoprotein |
| | ISCPNPLPFR EYRPQTEGVS NLVGLPNNIC LQKTSNQILK | Truncated (Gc Δ |
| | PKLISYTLPV VGQSGTCITD PLLAMDEGYF AYSHLERIGS | 34) Without N- |
| | CSRGVSKQRI IGVGEVLDRG DEVPSLFMTN VWTPPNPNTV | terminal |
| | YHCSAVYNNE FYYVLCAVST VGDPILNSTY WSGSLMMTRL | methionine |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRFNTC PEICWEGVYN DAFLIDRINW | |
| | ISAGVFLDSN QTAENPVFTV FKDNEILYRA QLASEDTNAQ | |
| | KTITNCFLLK NKIWCISLVE IYDTGDNVIR PKLFAVKIPE QCT | |
| 54 | LSQLQKNYLDNSNQQGDKMNNPDKKLSVNFNPLELDKGQKDLNKSYYVKNK | gb: JQ001776: 81 |
| | NYNVSNLLNESLHDIKFCIYCIFSLLIIITIINIITISIVITRLKVHEENN | 70- |
| | GMESPNLQSIQDSLSSLTNMINTEITPRIGILVTATSVTLSSSINYVGTKT | 10275\|Organism: |
| | NQLVNELKDYITKSCGFKVPELKLHECNISCADPKISKSAMYSTNAYAELA | Cedar |
| | GPPKIFCKSVSKDPDFRLKQIDYVIPVQQDRSICMNNPLLDISDGFFTYIH | virus\|Strain |
| | YEGINSCKKSDSFKVLLSHGEIVDRGDYRPSLYLLSSHYHPYSMQVINCVP | Name: CG1a\|Prot |
| | VTCNQSSFVFCHISNNTKTLDNSDYSSDEYYITYFNGIDRPKTKKIPINNM | ein |
| | TADNRYIHFTFSGGGGVCLGEEFIIPVTTVINTDVFTHDYCESFNCSVQTG | Name: attachmen |
| | KSLKEICSESLRSPTNSSRYNLNGIMIISQNNMTDFKIQLNGITYNKLSFG | t |
| | SPGRLSKTLGQVLYYQSSMSWDTYLKAGFVEKWKPFTPNWMNNTVISRPNQ | glycoprotein\|Gen |
| | GNCPRYHKCPEICYGGTYNDIAPLDLGKDMYVSVILDSDQLAENPEITVFN | e Symbol: G |
| | STTILYKERVSKDELNTRSTTTSCFLFLDEPWCISVLETNRFNGKSIRPEI | Without N- |
| | YSYKIPKYC | terminal |
| | | methionine |
| 55 | PQKTVEFINMNSPLERGVSTLSDKKTLNQSKITKQGYFGLGSHSERNWKKQ | gb: NC_025256: 9 |
| | KNQNDHYMTVSTMILEILVVLGIMFNLIVLTMVYYQNDNINQRMAELTSNI | 117- |
| | TVLNLNLNQLTNKIQREIIPRITLIDTATTITIPSAITYILATLTTRISEL | 11015\|Organism: |
| | LPSINQKCEFKTPTLVLNDCRINCTPPLNPSDGVKMSSLATNLVAHGPSPC | Bat |
| | RNFSSVPTIYYYRIPGLYNRTALDERCILNPRLTISSTKFAYVHSEYDKNC | Paramyxovirus |
| | TRGFKYYELMTFGEILEGPEKEPRMFSRSFYSPTNAVNYHSCTPIVTVNEG | Eid_hel/GH- |
| | YFLCLECTSSDPLYKANLSNSTFHLVILRHNKDEKIVSMPSFNLSTDQEYV | M74a/GHA/200 |
| | QIIPAEGGGTAESGNLYFPCIGRLLHKRVTHPLCKKSNCSRTDDESCLKSY | 9\|Strain |
| | YNQGSPQHQVVNCLIRIRNAQRDNPTWDVITVDLTNTYPGSRSRIFGSFSK | Name: BatPV/Ei |
| | PMLYQSSVSWHTLLQVAEITDLDKYQLDWLDTPYISRPGGSECPFGNYCPT | d_hel/GH- |
| | VCWEGTYNDVYSLTPNNDLFVTVYLKSEQVAENPYFAIFSRDQILKEFPLD | M74a/GHA/200 |
| | AWISSARTTTISCFMFNNEIWCIAALEITRLNDDIIRPIYYSFWLPTDCRT | 9\|Protein |
| | PYPHTGKMTRVPLRSTYNY | Name: glycoprote |
| | | in\|Gene |
| | | Symbol: G |
| | | Without N- |
| | | terminal |
| | | methionine |
| 56 | ATNRDNTITSAEVSQEDKVKKYYGVETAEKVADSISGNKVFILMNTLLILT | gb: NC_025352: 8 |
| | GAIITITLNITNLTAAKSQQNMLKIIQDDVNAKLEMFVNLDQLVKGEIKPK | 716- |
| | VSLINTAVSVSIPGQISNLQTKFLQKYVYLEESITKQCTCNPLSGIFPTSG | 11257\|Organism: |
| | PTYPPTDKPDDDTTDDDKVDTTIKPIEYPKPDGCNRTGDHFTMEPGANFYT | Mojiang |
| | VPNLGPASSNSDECYTNPSFSIGSSIYMFSQEIRKTDCTAGEILSIQIVLG | virus\|Strain |
| | RIVDKGQQGPQASPLLVWAVPNPKIINSCAVAAGDEMGWVLCSVTLTAASG | Name: Tongguan |
| | EPIPHMFDGFWLYKLEPDTEVVSYRITGYAYLLDKQYDSVFIGKGGGIQKG | 1\|Protein |
| | NDLYFQMYGLSRNRQSFKALCEHGSCLGTGGGGYQVLCDRAVMSFGSEESL | Name: attachmen |
| | ITNAYLKVNDLASGKPVIIGQTFPPSDSYKGSNGRMYTIGDKYGLYLAPSS | t |
| | WNRYLRFGITPDISVRSTTWLKSQDPIMKILSTCTNTDRDMCPEICNTRGY | glycoprotein\|lGen |
| | QDIFPLSEDSEYYTYIGITPNNGGTKNFVAVRDSDGHIASIDILQNYYSIT | e Symbol: G |
| | SATISCFMYKDEIWCIAITEGKKQKDNPQRIYAHSYKIRQMCYNMKSATVT | Without N- |
| | VGNAKNITIRRY | terminal |
| | | methionine |
| 57 | DFDKLNKIGVVQGRVLNYKIKGDPMTKDLVLKFIPNIVNITECVREPLSRY | gb: JQ001776: 61 |
| | NETVRRLLLPIHNMLGLYLNNTNAKMTGLMIAGVIMGGIAIGIATAAQITA | 29- |
| | GFALYEAKKNTENIQKLTDSIMKTQDSIDKLTDSVGTSILILNKLQTYINN | 8166\|Organism: |
| | QLVPNLELLSCRQNKIEFDLMLTKYLVDLMTVIGPNINNPVNKDMTIQSLS | Cedar |
| | LLFDGNYDIMMSELGYTPQDFLDLIESKSITGQIIYVDMENLYVVIRTYLP | virus\|Strain |
| | TLIEVPDAQIYEFNKITMSSNGGEYLSTIPNFILIRGNYMSNIDVATCYMT | Name: CG1a\|Prot |
| | KASVICNQDYSLPMSQNLRSCYQGETEYCPVEAVIASHSPRFALTNGVIFA | ein Name: fusion |
| | NCINTICRCQDNGKTITQNINQFVSMIDNSTCNDVMVDKFTIKVGKYMGRK | glycoprotein\|Gen |
| | DINNINIQIGPQIIIDKVDLSNEINKMNQSLKDSIFYLREAKRILDSVNIS | e Symbol: F |
| | LISPSVQLFLIIISVLSFIILLIIIVYLYCKSKHSYKYNKFIDDPDYYNDY | (without signal |
| | KRERINGKASKSNNIYYVGD | sequence) |
| 58 | SRALLRETDNYSNGLIVENLVRNCHHPSKNNLNYTKTQKRDSTIPYRVEER | gb: NC_025256: 6 |
| | KGHYPKIKHLIDKSYKHIKRGKRRNGHNGNIITIILLLILILKTQMSEGAI | 865- |
| | HYETLSKIGLIKGITREYKVKGTPSSKDIVIKLIPNVTGLNKCTNISMENY | 8853\|Organism: |
| | KEQLDKILIPINNIIELYANSTKSAPGNARFAGVIIAGVALGVAAAAQITA | Bat |
| | GIALHEARQNAERINLLKDSISATNNAVAELQEATGGIVNVITGMQDYINT | Paramyxovirus |
| | NLVPQIDKLQCSQIKTALDISLSQYYSEILTVFGPNLQNPVTTSMSIQAIS | Eid_hel/GH- |
| | QSFGGNIDLLLNLLGYTANDLLDLLESKSITGQITYINLEHYFMVIRVYYP | M74a/GHA/200 |
| | IMTTISNAYVQELIKISFNVDGSEWVSLVPSYILIRNSYLSNIDISECLIT | 9\|Strain |
| | KNSVICRHDFAMPMSYTLKECLTGDTEKCPREAVVTSYVPRFAISGGVIYA | Name: BatPV/Ei |
| | NCLSTTCQCYQTGKVIAQDGSQTLMMIDNQTCSIVRIEEILISTGKYLGSQ | d_hel/GH- |
| | EYNTMHVSVGNPVFTDKLDITSQISNINQSIEQSKFYLDKSKAILDKINLN | M74a/GHA/200 |
| | LIGSVPISILFIIAILSLILSIITFVIVMIIVRRYNKYTPLINSDPSSRRS | 9\|Protein |
| | TIQDVYIIPNPGEHSIRSAARSIDRDRD | Name: fusion |
| | | proteinlGene |
| | | Symbol: F |
| | | (without signal |
| | | sequence) |
| 59 | ILHY EKLSKIGLVK GITRKYKIKS | Hendra virus F |
| | NPLTKDIVIK MIPNVSNVSK CTGTVMENYK SRLTGILSPI | protein |
| | KGAIELYNNN | Uniprot O89342 |
| | THDLVGDVKL AGVVMAGIAI GIATAAQITA GVALYEAMKN | (without signal |
| | ADNINKLKSS | sequence) |
| | IESTNEAVVK LQETAEKTVY VLTALQDYIN TNLVPTIDQI | |
| | SCKQTELALD | |
| | LALSKYLSDL LFVFGPNLQD PVSNSMTIQA ISQAFGGNYE | |
| | TLLRTLGYAT EDFDDLLESD SIAGQIVYVD LSSYYIIVRV | |
| | YFPILTEIQQ AYVQELLPVS | |
| | FNNDNSEWIS IVPNFVLIRN TLISNIEVKY CLITKKSVIC | |
| | NQDYATPMTA | |
| | SVRECLTGST DKCPRELVVS SHVPRFALSG GVLFANCISV | |
| | TCQCQTTGRA ISQSGEQTLL MIDNTTCTTV VLGNIIISLG | |
| | KYLGSINYNS ESIAVGPPVY | |
| | TDKVDISSQI SSMNQSLQQS KDYIKEAQKI LDTVNPSLIS | |
| | MLSMIILYVL | |
| | SIAALCIGLI TFISFVIVEK KRGNYSRLDD RQVRPVSNGD LYYIGT | |
| 60 | IHYDSLSKVGVIKGLTYNYKIKGSPSTKLMVVKLIPNIDSVKNCTQKQYDE | gb: NC_025352: 5 |
| | YKNLVRKALEPVKMAIDTMLNNVKSGNNKYRFAGAIMAGVALGVATAATVT | 950- |
| | AGIALHRSNENAQAIANMKSAIQNTNEAVKQLQLANKQTLAVIDTIRGEIN | 8712\|Organism: |
| | NNIIPVINQLSCDTIGLSVGIRLTQYYSEIITAFGPALQNPVNTRITIQAI | Mojiang |
| | SSVFNGNFDELLKIMGYTSGDLYEILHSELIRGNIIDVDVDAGYIALEIEF | virus\|Strain |
| | PNLTLVPNAVVQELMPISYNIDGDEWVILVPRFVLTRTTLLSNIDTSRCTI | Name: Tongguan |
| | TDSSVICDNDYALPMSHELIGCLQGDTSKCAREKVVSSYVPKFALSDGLVY | 1\|Protein |
| | ANCLNTICRCMDTDTPISQSLGATVSLLDNKRCSVYQVGDVLISVGSYLGD | Name: fusion |
| | GEYNADNVELGPPIVIDKIDIGNQLAGINQTLQEAEDYIEKSEEFLKGVNP | protein\|Gene |
| | SIITLGSMVVLYIFMILIAIVSVIALVLSIKLTVKGNVVRQQFTYTQHVPS | Symbol: F |
| | MENINYVSH | (without signal |
| | | sequence) |
| 61 | MLFNLRILLNNAAFRNGHNFMVRNFRCGQPLQNKVQLKGRDLLTL | OTC |
| | KNFTGEEIKYMLWLSADLKFRIKQKGEYLPLLQGKSLGMIFEKRSTR | |
| | TRLSTETGFALLGGHPCFLTTQDIHLGVNESLTDTARVLSSMADAVL | |
| | ARVYKQSDLDTLAKEASIPIINGLSDLYHPIQILADYLTLQEHYSSLK | |
| | GLTLSWIGDGNNILHSIMMSAAKFGMHLQAATPKGYEPDASVTKL | |
| | AEQYAKENGTKLLLTNDPLEAAHGGNVLITDTWISMGQEEEKKKR | |
| | LQAFQGYQVTMKTAKVAASDWTFLHCLPRKPEEVDDEVFYSPRSL | |
| | VFPEAENRKWTIMAVMVSLLTDYSPQLQKPKF | |
| 62 | MTRILTAFKVVRTLKTGFGFTNVTAHQKWKFSRPGIRLLSVKAQTA | CPS1 |
| | HIVLEDGTKMKGYSFGHPSSVAGEVVFNTGLGGYPEAITDPAYKGQ | |
| | ILTMANPIIGNGGAPDTTALDELGLSKYLESNGIKVSGLLVLDYSKD | |
| | YNHWLATKSLGQWLQEEKVPAIYGVDTRMLTKIIRDKGTMLGKIEF | |
| | EGQPVDFVDPNKQNLIAEVSTKDVKVYGKGNPTKVVAVDCGIKNN | |
| | VIRLLVKRGAEVHLVPWNHDFTKMEYDGILIAGGPGNPALAEPLIQ | |
| | NVRKILESDRKEPLFGISTGNLITGLAAGAKTYKMSMANRGQNQPV | |
| | LNITNKQAFITAQNHGYALDNTLPAGWKPLFVNVNDQTNEGIMHES | |
| | KPFFAVQFHPEVTPGPIDTEYLFDSFFSLIKKGKATTITSVLPKPALVA | |
| | SRVEVSKVLILGSGGLSIGQAGEFDYSGSQAVKAMKEENVKTVLMN | |
| | PNIASVQTNEVGLKQADTVYFLPITPQFVTEVIKAEQPDGLILGMGG | |
| | QTALNCGVELFKRGVLKEYGVKVLGTSVESIMATEDRQLFSDKLNE | |
| | INEKIAPSFAVESIEDALKAADTIGYPVMIRSAYALGGLGSGICPNRE | |
| | TLMDLSTKAFAMTNQILVEKSVTGWKEIEYEVVRDADDNCVTVCN | |
| | MENVDAMGVHTGDSVVVAPAQTLSNAEFQMLRRTSINVVRHLGIV | |
| | GECNIQFALHPTSMEYCIIEVNARLSRSSALASKATGYPLAFIAAKIA | |
| | LGIPLPEIKNVVSGKTSACFEPSLDYMVTKIPRWDLDRFHGTSSRIGS | |
| | SMKSVGEVMAIGRTFEESFQKALRMCHPSIEGFTPRLPMNKEWPSN | |
| | LDLRKELSEPSSTRIYAIAKAIDDNMSLDEIEKLTYIDKWFLYKMRDI | |
| | LNMEKTLKGLNSESMTEETLKRAKEIGFSDKQISKCLGLTEAQTREL | |
| | RLKKNIHPWVKQIDTLAAEYPSVTNYLYVTYNGQEHDVNFDDHGM | |
| | MVLGCGPYHIGSSVEFDWCAVSSIRTLRQLGKKTVVVNCNPETVST | |
| | DFDECDKLYFEELSLERILDIYHQEACGGCIISVGGQIPNNLAVPLYK | |
| | NGVKIMGTSPLQIDRAEDRSIFSAVLDELKVAQAPWKAVNTLNEAL | |
| | EFAKSVDYPCLLRPSYVLSGSAMNVVFSEDEMKKFLEEATRVSQEH | |
| | PVVLTKFVEGAREVEMDAVGKDGRVISHAISEHVEDAGVHSGDAT | |
| | LMLPTQTISQGAIEKVKDATRKIAKAFAISGPFNVQFLVKGNDVLVI | |
| | ECNLRASRSFPFVSKTLGVDFIDVATKVMIGENVDEKHLPTLDHPIIP | |
| | ADYVAIKAPMFSWPRLRDADPILRCEMASTGEVACFGEGIHTAFLK | |
| | AMLSTGFKIPQKGILIGIQQSFRPRFLGVAEQLHNEGFKLFATEATSD | |
| | WLNANNVPATPVAWPSQEGQNPSLSSIRKLIRDGSIDLVINLPNNNT | |
| | KFVHDNYVIRRTAVDSGIPLLTNFQVTKLFAEAVQKSRKVDSKSLF | |
| | HYRQYSAGKAA | |
| 63 | MATALMAVVLRAAAVAPRLRGRGGTGGARRLSCGARRRAARGTS | NAGS |
| | PGRRLSTAWSQPQPPPEEYAGADDVSQSPVAEEPSWVPSPRPPVPHE | |
| | SPEPPSGRSLVQRDIQAFLNQCGASPGEARHWLTQFQTCHHSADKPF | |
| | AVIEVDEEVLKCQQGVSSLAFALAFLQRMDMKPLVVLGLPAPTAPS | |
| | GCLSFWEAKAQLAKSCKVLVDALRHNAAAAVPFFGGGSVLRAAEP | |
| | APHASYGGIVSVETDLLQWCLESGSIPILCPIGETAARRSVLLDSLEV | |
| | TASLAKALRPTKIIFLNNTGGLRDSSHKVLSNVNLPADLDLVCNAE | |
| | WVSTKERQQMRLIVDVLSRLPHHSSAVITAASTLLTELFSNKGSGTL | |
| | FKNAERMLRVRSLDKLDQGRLVDLVNASFGKKLRDDYLASLRPRL | |
| | HSIYVSEGYNAAAILTMEPVLGGTPYLDKFVVSSSRQGQGSGQMLW | |
| | ECLRRDLQTLFWRSRVTNPINPWYFKHSDGSFSNKQWIFFWFGLAD | |
| | IRDSYELVNHAKGLPDSFHKPASDPGS | |
| 64 | MAVAIAAARVWRLNRGLSQAALLLLRQPGARGLARSHPPRQQQQF | BCKDHA |
| | SSLDDKPQFPGASAEFIDKLEFIQPNVISGIPIYRVMDRQGQIINPSEDP | |
| | HLPKEKVLKLYKSMTLLNTMDRILYESQRQGRISFYMTNYGEEGTH | |
| | VGSAAALDNTDLVFGQYREAGVLMYRDYPLELFMAQCYGNISDLG | |
| | KGRQMPVHYGCKERHFVTISSPLATQIPQAVGAAYAAKRANANRV | |
| | VICYFGEGAASEGDAHAGFNFAATLECPIIFFCRNNGYAISTPTSEQY | |
| | RGDGIAARGPGYGIMSIRVDGNDVFAVYNATKEARRRAVAENQPF | |
| | LIEAMTYRIGHHSTSDDSSAYRSVDEVNYWDKQDHPISRLRHYLLS | |
| | QGWWDEEQEKAWRKQSRRKVMEAFEQAERKPKPNPNLLFSDVYQ | |
| | EMPAQLRKQQESLARHLQTYGEHYPLDHFDK | |
| 65 | MAVVAAAAGWLLRLRAAGAEGHWRRLPGAGLARGFLHPAATVE | BCKDHB |
| | DAAQRRQVAHFTFQPDPEPREYGQTQKMNLFQSVTSALDNSLAKD | |
| | PTAVIFGEDVAFGGVFRCTVGLRDKYGKDRVFNTPLCEQGIVGFGIG | |
| | IAVTGATAIAEIQFADYIFPAFDQIVNEAAKYRYRSGDLFNCGSLTIR | |
| | SPWGCVGHGALYHSQSPEAFFAHCPGIKVVIPRSPFQAKGLLLSCIE | |
| | DKNPCIFFEPKILYRAAAEEVPIEPYNIPLSQAEVIQEGSDVTLVAWG | |
| | TQVHVIREVASMAKEKLGVSCEVIDLRTIIPWDVDTICKSVIKTGRLL | |
| | ISHEAPLTGGFASEISSTVQEECFLNLEAPISRVCGYDTPFPHIFEPFYI | |
| | PDKWKCYDALRKMINY | |
| 66 | MAAVRMLRTWSRNAGKLICVRYFQTCGNVHVLKPNYVCFFGYPSF | DBT |
| | KYSHPHHFLKTTAALRGQVVQFKLSDIGEGIREVTVKEWYVKEGDT | |
| | VSQFDSICEVQSDKASVTITSRYDGVIKKLYYNLDDIAYVGKPLVDI | |
| | ETEALKDSEEDVVETPAVSHDEHTHQEIKGRKTLATPAVRRLAMEN | |
| | NIKLSEVVGSGKDGRILKEDILNYLEKQTGAILPPSPKVEIMPPPPKP | |
| | KDMTVPILVSKPPVFTGKDKTEPIKGFQKAMVKTMSAALKIPHFGY | |
| | CDEIDLTELVKLREELKPIAFARGIKLSFMPFFLKAASLGLLQFPILNA | |
| | SVDENCQNITYKASHNIGIAMDTEQGLIVPNVKNVQICSIFDIATELN | |
| | RLQKLGSVGQLSTTDLTGGTFTLSNIGSIGGTFAKPVIMPPEVAIGAL | |
| | GSIKAIPRFNQKGEVYKAQIMNVSWSADHRVIDGATMSRFSNLWKS | |
| | YLENPAFMLLDLK | |
| 67 | MQSWSRVYCSLAKRGHFNRISHGLQGLSAVPLRTYADQPIDADVTV | DLD |
| | IGSGPGGYVAAIKAAQLGFKTVCIEKNETLGGTCLNVGCIPSKALLN | |
| | NSHYYHMAHGKDFASRGIEMSEVRLNLDKMMEQKSTAVKALTGGI | |
| | AHLFKQNKVVHVNGYGKITGKNQVTATKADGGTQVIDTKNILIATG | |
| | SEVTPFPGITIDEDTIVSSTGALSLKKVPEKMVVIGAGVIGVELGSVW | |
| | QRLGADVTAVEFLGHVGGVGIDMEISKNFQRILQKQGFKFKLNTKV | |
| | TGATKKSDGKIDVSIEAASGGKAEVITCDVLLVCIGRRPFTKNLGLE | |
| | ELGIELDPRGRIPVNTRFQTKIPNIYAIGDVVAGPMLAHKAEDEGIIC | |
| | VEGMAGGAVHIDYNCVPSVIYTHPEVAWVGKSEEQLKEEGIEYKV | |
| | GKFPFAANSRAKTNADTDGMVKILGQKSTDRVLGAHILGPGAGEM | |
| | VNEAALALEYGASCEDIARVCHAHPTLSEAFREANLAASFGKSINF | |
| 68 | MLRAKNQLFLLSPHYLRQVKESSGSRLIQQRLLHQQQPLHPEWAAL | MUT |
| | AKKQLKGKNPEDLIWHTPEGISIKPLYSKRDTMDLPEELPGVKPFTR | |
| | GPYPTMYTFRPWTIRQYAGFSTVEESNKFYKDNIKAGQQGLSVAFD | |
| | LATHRGYDSDNPRVRGDVGMAGVAIDTVEDTKILFDGIPLEKMSVS | |
| | MTMNGAVIPVLANFIVTGEEQGVPKEKLTGTIQNDILKEFMVRNTYI | |
| | FPPEPSMKIIADIFEYTAKHMPKFNSISISGYHMQEAGADAILELAYT | |
| | LADGLEYSRTGLQAGLTIDEFAPRLSFFWGIGMNFYMEIAKMRAGR | |
| | RLWAHLIEKMFQPKNSKSLLLRAHCQTSGWSLTEQDPYNNIVRTAI | |
| | EAMAAVFGGTQSLHTNSFDEALGLPTVKSARIARNTQIIIQEESGIPK | |
| | VADPWGGSYMMECLTNDVYDAALKLINEIEEMGGMAKAVAEGIP | |
| | KLRIEECAARRQARIDSGSEVIVGVNKYQLEKEDAVEVLAIDNTSVR | |
| | NRQIEKLKKIKSSRDQALAERCLAALTECAASGDGNILALAVDASR | |
| | ARCTVGEITDALKKVFGEHKANDRMVSGAYRQEFGESKEITSAIKR | |
| | VHKFMEREGRRPRLLVAKMGQDGHDRGAKVIATGFADLGFDVDIG | |
| | PLFQTPREVAQQAVDADVHAVGISTLAAGHKTLVPELIKELNSLGRP | |
| | DILVMCGGVIPPQDYEFLFEVGVSNVFGPGTRIPKAAVQVLDDIEKC | |
| | LEKKQQSV | |
| 69 | MPMLLPHPHQHFLKGLLRAPFRCYHFIFHSSTHLGSGIPCAQPFNSL | MMAA |
| | GLHCTKWMLLSDGLKRKLCVQTTLKDHTEGLSDKEQRFVDKLYTG | |
| | LIQGQRACLAEAITLVESTHSRKKELAQVLLQKVLLYHREQEQSNK | |
| | GKPLAFRVGLSGPPGAGKSTFIEYFGKMLTERGHKLSVLAVDPSSCT | |
| | SGGSLLGDKTRMTELSRDMNAYIRPSPTRGTLGGVTRTTNEAILLCE | |
| | GAGYDIILIETVGVGQSEFAVADMVDMFVLLLPPAGGDELQGIKRGI | |
| | IEMADLVAVTKSDGDLIVPARRIQAEYVSALKLLRKRSQVWKPKVI | |
| | RISARSGEGISEMWDKMKDFQDLMLASGELTAKRRKQQKVWMWN | |
| | LIQESVLEHFRTHPTVREQIPLLEQKVLIGALSPGLAADFLLKAFKSR | |
| | D | |
| 70 | MAVCGLGSRLGLGSRLGLRGCFGAARLLYPRFQSRGPQGVEDGDR | MMAB |
| | PQPSSKTPRIPKIYTKTGDKGFSSTFTGERRPKDDQVFEAVGTTDELS | |
| | SAIGFALELVTEKGHTFAEELQKIQCTLQDVGSALATPCSSAREAHL | |
| | KYTTFKAGPILELEQWIDKYTSQLPPLTAFILPSGGKISSALHFCRAV | |
| | CRRAERRVVPLVQMGETDANVAKFLNRLSDYLFTLARYAAMKEG | |
| | NQEKIYMKNDPSAESEGL | |
| 71 | MFDRALKPFLQSCHLRMLTDPVDQCVAYHLGRVRESLPELQIEIIAD | MMACHC |
| | YEVHPNRRPKILAQTAAHVAGAAYYYQRQDVEADPWGNQRISGVC | |
| | IHPRFGGWFAIRGVVLLPGIEVPDLPPRKPHDCVPTRADRIALLEGFN | |
| | FHWRDWTYRDAVTPQERYSEEQKAYFSTPPAQRLALLGLAQPSEKP | |
| | SSPSPDLPFTTPAPKKPGNPSRARSWLSPRVSPPASPGP | |
| 72 | MANVLCNRARLVSYLPGFCSLVKRVVNPKAFSTAGSSGSDESHVA | MMADHC |
| | AAPPDICSRTVWPDETMGPFGPQDQRFQLPGNIGFDCHLNGTASQK | |
| | KSLVHKTLPDVLAEPLSSERHEFVMAQYVNEFQGNDAPVEQEINSA | |
| | ETYFESARVECAIQTCPELLRKDFESLFPEVANGKLMILTVTQKTKN | |
| | DMTVWSEEVEIEREVLLEKFINGAKEICYALRAEGYWADFIDPSSGL | |
| | AFFGPYTNNTLFETDERYRHLGFSVDDLGCCKVIRHSLWGTHVVVG | |
| | SIFTNATPDSHIMKKLSGN | |
| 73 | MARVLKAAAANAVGLFSRLQAPIPTVRASSTSQPLDQVTGSVWNL | MCEE |
| | GRLNHVAIAVPDLEKAAAFYKNILGAQVSEAVPLPEHGVSVVFVNL | |
| | GNTKMELLHPLGRDSPIAGFLQKNKAGGMHHICIEVDNINAAVMDL | |
| | KKKKIRSLSEEVKIGAHGKPVIFLHPKDCGGVLVELEQA | |
| 74 | MAGFWVGTAPLVAAGRRGRWPPQQLMLSAALRTLKHVLYYSRQC | PCCA |
| | LMVSRNLGSVGYDPNEKTFDKILVANRGEIACRVIRTCKKMGIKTV | |
| | AIHSDVDASSVHVKMADEAVCVGPAPTSKSYLNMDAIMEAIKKTR | |
| | AQAVHPGYGFLSENKEFARCLAAEDVVFIGPDTHAIQAMGDKIESK | |
| | LLAKKAEVNTIPGFDGVVKDAEEAVRIAREIGYPVMIKASAGGGGK | |
| | GMRIAWDDEETRDGFRLSSQEAASSFGDDRLLIEKFIDNPRHIEIQVL | |
| | GDKHGNALWLNERECSIQRRNQKVVEEAPSIFLDAETRRAMGEQA | |
| | VALARAVKYSSAGTVEFLVDSKKNFYFLEMNTRLQVEHPVTECITG | |
| | LDLVQEMIRVAKGYPLRHKQADIRINGWAVECRVYAEDPYKSFGLP | |
| | SIGRLSQYQEPLHLPGVRVDSGIQPGSDISIYYDPMISKLITYGSDRTE | |
| | ALKRMADALDNYVIRGVTHNIALLREVIINSRFVKGDISTKFLSDVY | |
| | PDGFKGHMLTKSEKNQLLAIASSLFVAFQLRAQHFQENSRMPVIKP | |
| | DIANWELSVKLHDKVHTVVASNNGSVFSVEVDGSKLNVTSTWNLA | |
| | SPLLSVSVDGTQRTVQCLSREAGGNMSIQFLGTVYKVNILTRLAAEL | |
| | NKFMLEKVTEDTSSVLRSPMPGVVVAVSVKPGDAVAEGQEICVIEA | |
| | MKMQNSMTAGKTGTVKSVHCQAGDTVGEGDLLVELE | |
| 75 | MAAALRVAAVGARLSVLASGLRAAVRSLCSQATSVNERIENKRRT | PCCB |
| | ALLGGGQRRIDAQHKRGKLTARERISLLLDPGSFVESDMFVEHRCA | |
| | DFGMAADKNKFPGDSVVTGRGRINGRLVYVFSQDFTVFGGSLSGA | |
| | HAQKICKIMDQAITVGAPVIGLNDSGGARIQEGVESLAGYADIFLRN | |
| | VTASGVIPQISLIMGPCAGGAVYSPALTDFTFMVKDTSYLFITGPDV | |
| | VKSVTNEDVTQEELGGAKTHTTMSGVAHRAFENDVDALCNLRDFF | |
| | NYLPLSSQDPAPVRECHDPSDRLVPELDTIVPLESTKAYNMVDIIHSV | |
| | VDEREFFEIMPNYAKNIIVGFARMNGRTVGIVGNQPKVASGCLDINS | |
| | SVKGARFVRFCDAFNIPLITFVDVPGFLPGTAQEYGGIIRHGAKLLY | |
| | AFAEATVPKVTVITRKAYGGAYDVMSSKHLCGDTNYAWPTAEIAV | |
| | MGAKGAVEIIFKGHENVEAAQAEYIEKFANPFPAAVRGFVDDIIQPS | |
| | STRARICCDLDVLASKKVQRPWRKHANIPL | |
| 76 | MAVESQGGRPLVLGLLLCVLGPVVSHAGKILLIPVDGSHWLSMLGA | UGT1A1 |
| | IQQLQQRGHEIVVLAPDASLYIRDGAFYTLKTYPVPFQREDVKESFV | |
| | SLGHNVFENDSFLQRVIKTYKKIKKDSAMLLSGCSHLLHNKELMAS | |
| | LAESSFDVMLTDPFLPCSPIVAQYLSLPTVFFLHALPCSLEFEATQCP | |
| | NPFSYVPRPLSSHSDHMTFLQRVKNMLIAFSQNFLCDVVYSPYATL | |
| | ASEFLQREVTVQDLLSSASVWLFRSDFVKDYPRPIMPNMVFVGGIN | |
| | CLHQNPLSQEFEAYINASGEHGIVVFSLGSMVSEIPEKKAMAIADAL | |
| | GKIPQTVLWRYTGTRPSNLANNTILVKWLPQNDLLGHPMTRAFITH | |
| | AGSHGVYESICNGVPMVMMPLFGDQMDNAKRMETKGAGVTLNVL | |
| | EMTSEDLENALKAVINDKSYKENIMRLSSLHKDRPVEPLDLAVFWV | |
| | EFVMRHKGAPHLRPAAHDLTWYQYHSLDVIGFLLAVVLTVAFITFK | |
| | CCAYGYRKCLGKKGRVKKAHKSKTH | |
| 77 | MSSKGSVVLAYSGGLDTSCILVWLKEQGYDVIAYLANIGQKEDFEE | ASS1 |
| | ARKKALKLGAKKVFIEDVSREFVEEFIWPAIQSSALYEDRYLLGTSL | |
| | ARPCIARKQVEIAQREGAKYVSHGATGKGNDQVRFELSCYSLAPQI | |
| | KVIAPWRMPEFYNRFKGRNDLMEYAKQHGIPIPVTPKNPWSMDEN | |
| | LMHISYEAGILENPKNQAPPGLYTKTQDPAKAPNTPDILEIEFKKGVP | |
| | VKVTNVKDGTTHQTSLELFMYLNEVAGKHGVGRIDIVENRFIGMKS | |
| | RGIYETPAGTILYHAHLDIEAFTMDREVRKIKQGLGLKFAELVYTGF | |
| | WHSPECEFVRHCIAKSQERVEGKVQVSVLKGQVYILGRESPLSLYN | |
| | EELVSMNVQGDYEPTDATGFININSLRLKEYHRLQSKVTAK | |
| 78 | MSTAVLENPGLGRKLSDFGQETSYIEDNCNQNGAISLIFSLKEEVGA | PAH |
| | LAKVLRLFEENDVNLTHIESRPSRLKKDEYEFFTHLDKRSLPALTNII | |
| | KILRHDIGATVHELSRDKKKDTVPWFPRTIQELDRFANQILSYGAEL | |
| | DADHPGFKDPVYRARRKQFADIAYNYRHGQPIPRVEYMEEEKKTW | |
| | GTVFKTLKSLYKTHACYEYNHIFPLLEKYCGFHEDNIPQLEDVSQFL | |
| | QTCTGFRLRPVAGLLSSRDFLGGLAFRVFHCTQYIRHGSKPMYTPEP | |
| | DICHELLGHVPLFSDRSFAQFSQEIGLASLGAPDEYIEKLATIYWFTV | |
| | EFGLCKQGDSIKAYGAGLLSSFGELQYCLSEKPKLLPLELEKTAIQN | |
| | YTVTEFQPLYYVAESFNDAKEKVRNFAATIPRPFSVRYDPYTQRIEV | |
| | LDNTQQLKILADSINSEIGILCSALQKIK | |
| 79 | MAKTLSQAQSKTSSQQFSFTGNSSANVIIGNQKLTINDVARVARNGT | PAL |
| | LVSLTNNTDILQGIQASCDYINNAVESGEPIYGVTSGFGGMANVAIS | |
| | REQASELQTNLVWFLKTGAGNKLPLADVRAAMLLRANSHMRGAS | |
| | GIRLELIKRMEIFLNAGVTPYVYEFGSIGASGDLVPLSYITGSLIGLDP | |
| | SFKVDFNGKEMDAPTALRQLNLSPLTLLPKEGLAMMNGTSVMTGI | |
| | AANCVYDTQILTAIAMGVHALDIQALNGTNQSFHPFIHNSKPHPGQL | |
| | WAADQMISLLANS | |
| | QLVRDELDGKHDYRDHELIQDRYSLRCLPQYLGPIVDGISQIAKQIEI | |
| | EINSVTDNPLIDVDNQASYHGGNFLGQYVGMGMDHLRYYIGLLAK | |
| | HLDVQIALLASPEFSNGLPPSLLGNRERKVNMGLKGLQICGNSIMPL | |
| | LTFYGNSIADRFPTHAEQFNQNINSQGYTSATLARRSVDIFQNYVAI | |
| | ALMFGVQAVDLRTYKKTGHYDARASLSPATERLYSAVRHVVGQKP | |
| | TSDRPYIWNDNEQGLDEHIARISADIAAGGVIVQAVQDILPSLH | |
| 80 | MSTERDSETTFDEDSQPNDEVVPYSDDETEDELDDQGSAVEPEQNR | ATP8B1 |
| | VNREAEENREPFRKECTWQVKANDRKYHEQPHFMNTKFLCIKESK | |
| | YANNAIKTYKYNAFTFIPMNLFEQFKRAANLYFLALLILQAVPQIST | |
| | LAWYTTLVPLLVVLGVTAIKDLVDDVARHKMDKEINNRTCEVIKD | |
| | GRFKVAKWKEIQVGDVIRLKKNDFVPADILLLSSSEPNSLCYVETAE | |
| | LDGETNLKFKMSLEITDQYLQREDTLATFDGFIECEEPNNRLDKFTG | |
| | TLFWRNTSFPLDADKILLRGCVIRNTDFCHGLVIFAGADTKIMKNSG | |
| | KTRFKRTKIDYLMNYMVYTIFVVLILLSAGLAIGHAYWEAQVGNSS | |
| | WYLYDGEDDTPSYRGFLIFWGYIIVLNTMVPISLYVSVEVIRLGQSH | |
| | FINWDLQMYYAEKDTPAKARTTTLNEQLGQIHYIFSDKTGTLTQNI | |
| | MTFKKCCINGQIYGDHRDASQHNHNKIEQVDFSWNTYADGKLAFY | |
| | DHYLIEQIQSGKEPEVRQFFFLLAVCHTVMVDRTDGQLNYQAASPD | |
| | EGALVNAARNFGFAFLARTQNTITISELGTERTYNVLAILDFNSDRK | |
| | RMSIIVRTPEGNIKLYCKGADTVIYERLHRMNPTKQETQDALDIFAN | |
| | ETLRTLCLCYKEIEEKEFTEWNKKFMAASVASTNRDEALDKVYEEI | |
| | EKDLILLGATAIEDKLQDGVPETISKLAKADIKIWVLTGDKKETAENI | |
| | GFACELLTEDTTICYGEDINSLLHARMENQRNRGGVYAKFAPPVQE | |
| | SFFPPGGNRALIITGSWLNEILLEKKTKRNKILKLKFPRTEEERRMRT | |
| | QSKRRLEAKKEQRQKNFVDLACECSAVICCRVTPKQKAMVVDLVK | |
| | RYKKAITLAIGDGANDVNMIKTAHIGVGISGQEGMQAVMSSDYSFA | |
| | QFRYLQRLLLVHGRWSYIRMCKFLRYFFYKNFAFTLVHFWYSFFNG | |
| | YSAQTAYEDWFITLYNVLYTSLPVLLMGLLDQDVSDKLSLRFPGLY | |
| | IVGQRDLLFNYKRFFVSLLHGVLTSMILFFIPLGAYLQTVGQDGEAP | |
| | SDYQSFAVTIASALVITVNFQIGLDTSYWTFVNAFSIFGSIALYFGIMF | |
| | DFHSAGIHVLFPSAFQFTGTASNALRQPYIWLTIILAVAVCLLPVVAI | |
| | RFLSMTIWPSESDKIQKHRKRLKAEEQWQRRQQVFRRGVSTRRSAY | |
| | AFSHQRGYADLISSGRSIRKKRSPLDAIVADGTAEYRRTGDS | |
| 81 | MSDSVILRSIKKFGEENDGFESDKSYNNDKKSRLQDEKKGDGVRVG | ABCB11 |
| | FFQLFRFSSSTDIWLMFVGSLCAFLHGIAQPGVLLIFGTMTDVFIDYD | |
| | VELQELQIPGKACVNNTIVWTNSSLNQNMTNGTRCGLLNIESEMIKF | |
| | ASYYAGIAVAVLITGYIQICFWVIAAARQIQKMRKFYFRRIMRMEIG | |
| | WFDCNSVGELNTRFSDDINKINDAIADQMALFIQRMTSTICGFLLGF | |
| | FRGWKLTLVIISVSPLIGIGAATIGLSVSKFTDYELKAYAKAGVVAD | |
| | EVISSMRTVAAFGGEKREVERYEKNLVFAQRWGIRKGIVMGFFTGF | |
| | VWCLIFLCYALAFWYGSTLVLDEGEYTPGTLVQIFLSVIVGALNLGN | |
| | ASPCLEAFATGRAAATSIFETIDRKPIIDCMSEDGYKLDRIKGEIEFHN | |
| | VTFHYPSRPEVKILNDLNMVIKPGEMTALVGPSGAGKSTALQLIQRF | |
| | YDPCEGMVTVDGHDIRSLNIQWLRDQIGIVEQEPVLFSTTIAENIRYG | |
| | REDATMEDIVQAAKEANAYNFIMDLPQQFDTLVGEGGGQMSGGQ | |
| | KQRVAIARALIRNPKILLLDMATSALDNESEAMVQEVLSKIQHGHTII | |
| | SVAHRLSTVRAADTIIGFEHGTAVERGTHEELLERKGVYFTLVTLQS | |
| | QGNQALNEEDIKDATEDDMLARTFSRGSYQDSLRASIRQRSKSQLS | |
| | YLVHEPPLAVVDHKSTYEEDRKDKDIPVQEEVEPAPVRRILKFSAPE | |
| | WPYMLVGSVGAAVNGTVTPLYAFLFSQILGTFSIPDKEEQRSQINGV | |
| | CLLFVAMGCVSLFTQFLQGYAFAKSGELLTKRLRKFGFRAMLGQDI | |
| | AWFDDLRNSPGALTTRLATDASQVQGAAGSQIGMIVNSFTNVTVA | |
| | MIIAFSFSWKLSLVILCFFPFLALSGATQTRMLTGFASRDKQALEMV | |
| | GQITNEALSNIRTVAGIGKERRHEALETELEKPFKTAIQKANIYGFCF | |
| | AFAQCIMFIANSASYRYGGYLISNEGLHFSYVFRVISAVVLSATALG | |
| | RAFSYTPSYAKAKISAARFFQLLDRQPPISVYNTAGEKWDNFQGKID | |
| | FVDCKFTYPSRPDSQVLNGLSVSISPGQTLAFVGSSGCGKSTSIQLLE | |
| | RFYDPDQGKVMIDGHDSKKVNVQFLRSNIGIVSQEPVLFACSIMDNI | |
| | KYGDNTKEIPMERVIAAAKQAQLHDFVMSLPEKYETNVGSQGSQLS | |
| | RGEKQRIAIARAIVRDPKILLLDEATSALDTESEKTVQVALDKAREG | |
| | RTCIVIAHRLSTIQNADIIAVMAQGVVIEKGTHEELMAQKGAYYKLV | |
| | TTGSPIS | |
| 82 | MDLEAAKNGTAWRPTSAEGDFELGISSKQKRKKTKTVKMIGVLTLF | ABCB4 |
| | RYSDWQDKLFMSLGTIMAIAHGSGLPLMMIVFGEMTDKFVDTAGN | |
| | FSFPVNFSLSLLNPGKILEEEMTRYAYYYSGLGAGVLVAAYIQVSFW | |
| | TLAAGRQIRKIRQKFFHAILRQEIGWFDINDTTELNTRLTDDISKISEG | |
| | IGDKVGMFFQAVATFFAGFIVGFIRGWKLTLVIMAISPILGLSAAVW | |
| | AKILSAFSDKELAAYAKAGAVAEEALGAIRTVIAFGGQNKELERYQ | |
| | KHLENAKEIGIKKAISANISMGIAFLLIYASYALAFWYGSTLVISKEY | |
| | TIGNAMTVFFSILIGAFSVGQAAPCIDAFANARGAAYVIFDIIDNNPKI | |
| | DSFSERGHKPDSIKGNLEFNDVHFSYPSRANVKILKGLNLKVQSGQT | |
| | VALVGSSGCGKSTTVQLIQRLYDPDEGTINIDGQDIRNFNVNYLREII | |
| | GVVSQEPVLFSTTIAENICYGRGNVTMDEIKKAVKEANAYEFIMKLP | |
| | QKFDTLVGERGAQLSGGQKQRIAIARALVRNPKILLLDEATSALDTE | |
| | SEAEVQAALDKAREGRTTIVIAHRLSTVRNADVIAGFEDGVIVEQGS | |
| | HSELMKKEGVYFKLVNMQTSGSQIQSEEFELNDEKAATRMAPNGW | |
| | KSRLFRHSTQKNLKNSQMCQKSLDVETDGLEANVPPVSFLKVLKLN | |
| | KTEWPYFVVGTVCAIANGGLQPAFSVIFSEIIAIFGPGDDAVKQQKC | |
| | NIFSLIFLFLGIISFFTFFLQGFTFGKAGEILTRRLRSMAFKAMLRQDM | |
| | SWFDDHKNSTGALSTRLATDAAQVQGATGTRLALIAQNIANLGTGII | |
| | ISFIYGWQLTLLLLAVVPIIAVSGIVEMKLLAGNAKRDKKELEAAGK | |
| | IATEAIENIRTVVSLTQERKFESMYVEKLYGPYRNSVQKAHIYGITFS | |
| | ISQAFMYFSYAGCFRFGAYLIVNGHMRFRDVILVFSAIVFGAVALGH | |
| | ASSFAPDYAKAKLSAAHLFMLFERQPLIDSYSEEGLKPDKFEGNITF | |
| | NEVVFNYPTRANVPVLQGLSLEVKKGQTLALVGSSGCGKSTVVQL | |
| | LERFYDPLAGTVFVDFGFQLLDGQEAKKLNVQWLRAQLGIVSQEPI | |
| | LFDCSIAENIAYGDNSRVVSQDEIVSAAKAANIHPFIETLPHKYETRV | |
| | GDKGTQLSGGQKQRIAIARALIRQPQILLLDEATSALDTESEKVVQE | |
| | ALDKAREGRTCIVIAHRLSTIQNADLIVVFQNGRVKEHGTHQQLLA | |
| | QKGIYFSMVSVQAGTQNL | |
| 83 | MPVRGDRGFPPRRELSGWLRAPGMEELIWEQYTVTLQKDSKRGFGI | TJP2 |
| | AVSGGRDNPHFENGETSIVISDVLPGGPADGLLQENDRVVMVNGTP | |
| | MEDVLHSFAVQQLRKSGKVAAIVVKRPRKVQVAALQASPPLDQDD | |
| | RAFEVMDEFDGRSFRSGYSERSRLNSHGGRSRSWEDSPERGRPHER | |
| | ARSRERDLSRDRSRGRSLERGLDQDHARTRDRSRGRSLERGLDHDF | |
| | GPSRDRDRDRSRGRSIDQDYERAYHRAYDPDYERAYSPEYRRGAR | |
| | HDARSRGPRSRSREHPHSRSPSPEPRGRPGPIGVLLMKSRANEEYGL | |
| | RLGSQIFVKEMTRTGLATKDGNLHEGDIILKINGTVTENMSLTDARK | |
| | LIEKSRGKLQLVVLRDSQQTLINIPSLNDSDSEIEDISEIESNRSFSPEE | |
| | RRHQYSDYDYHSSSEKLKERPSSREDTPSRLSRMGATPTPFKSTGDI | |
| | AGTVVPETNKEPRYQEDPPAPQPKAAPRTFLRPSPEDEAIYGPNTKM | |
| | VRFKKGDSVGLRLAGGNDVGIFVAGIQEGTSAEQEGLQEGDQILKV | |
| | NTQDFRGLVREDAVLYLLEIPKGEMVTILAQSRADVYRDILACGRG | |
| | DSFFIRSHFECEKETPQSLAFTRGEVFRVVDTLYDGKLGNWLAVRIG | |
| | NELEKGLIPNKSRAEQMASVQNAQRDNAGDRADFWRMRGQRSGV | |
| | KKNLRKSREDLTAVVSVSTKFPAYERVLLREAGFKRPVVLFGPIADI | |
| | AMEKLANELPDWFQTAKTEPKDAGSEKSTGVVRLNTVRQIIEQDKH | |
| | ALLDVTPKAVDLLNYTQWFPIVIFFNPDSRQGVKTMRQRLNPTSNK | |
| | SSRKLFDQANKLKKTCAHLFTATINLNSANDSWFGSLKDTIQHQQG | |
| | EAVWVSEGKMEGMDDDPEDRMSYLTAMGADYLSCDSRLISDFEDT | |
| | DGEGGAYTDNELDEPAEEPLVSSITRSSEPVQHEESIRKPSPEPRAQM | |
| | RRAASSDQLRDNSPPPAFKPEPPKAKTQNKEESYDFSKSYEYKSNPS | |
| | AVAGNETPGASTKGYPPPVAAKPTFGRSILKPSTPIPPQEGEEVGESS | |
| | EEQDNAPKSVLGKVKIFEKMDHKARLQRMQELQEAQNARIEIAQK | |
| | HPDIYAVPIKTHKPDPGTPQHTSSRPPEPQKAPSRPYQDTRGSYGSD | |
| | AEEEEYRQQLSEHSKRGYYGQSARYRDTEL | |
| 84 | MATATRLLGWRVASWRLRPPLAGFVSQRAHSLLPVDDAINGLSEE | IVD |
| | QRQLRQTMAKFLQEHLAPKAQEIDRSNEFKNLREFWKQLGNLGVL | |
| | GITAPVQYGGSGLGYLEHVLVMEEISRASGAVGLSYGAHSNLCINQ | |
| | LVRNGNEAQKEKYLPKLISGEYIGALAMSEPNAGSDVVSMKLKAE | |
| | KKGNHYILNGNKFWITNGPDADVLIVYAKTDLAAVPASRGITAFIVE | |
| | KGMPGFSTSKKLDKLGMRGSNTCELIFEDCKIPAANILGHENKGVY | |
| | VLMSGLDLERLVLAGGPLGLMQAVLDHTIPYLHVREAFGQKIGHFQ | |
| | LMQGKMADMYTRLMACRQYVYNVAKACDEGHCTAKDCAGVILY | |
| | SAECATQVALDGIQCFGGNGYINDFPMGRFLRDAKLYEIGAGTSEV | |
| | RRLVIGRAFNADFH | |
| 85 | MALRGVSVRLLSRGPGLHVLRTWVSSAAQTEKGGRTQSQLAKSSR | GCDH |
| | PEFDWQDPLVLEEQLTTDEILIRDTFRTYCQERLMPRILLANRNEVF | |
| | HREIISEMGELGVLGPTIKGYGCAGVSSVAYGLLARELERVDSGYRS | |
| | AMSVQSSLVMHPIYAYGSEEQRQKYLPQLAKGELLGCFGLTEPNSG | |
| | SDPSSMETRAHYNSSNKSYTLNGTKTWITNSPMADLFVVWARCED | |
| | GCIRGFLLEKGMRGLSAPRIQGKFSLRASATGMIIMDGVEVPEENVL | |
| | PGASSLGGPFGCLNNARYGIAWGVLGASEFCLHTARQYALDRMQF | |
| | GVPLARNQLIQKKLADMLTEITLGLHACLQLGRLKDQDKAAPEMV | |
| | SLLKRNNCGKALDIARQARDMLGGNGISDEYHVIRHAMNLEAVNT | |
| | YEGTHDIHALILGRAITGIQAFTASK | |
| 86 | MFRAAAPGQLRRAASLLRFQSTLVIAEHANDSLAPITLNTITAATRL | ETFA |
| | GGEVSCLVAGTKCDKVAQDLCKVAGIAKVLVAQHDVYKGLLPEEL | |
| | TPLILATQKQFNYTHICAGASAFGKNLLPRVAAKLEVAPISDHAIKSP | |
| | DTFVRTIYAGNALCTVKCDEKVKVFSVRGTSFDAAATSGGSASSEK | |
| | ASSTSPVEISEWLDQKLTKSDRPELTGAKVVVSGGRGLKSGENFKLL | |
| | YDLADQLHAAVGASRAAVDAGFVPNDMQVGQTGKIVAPELYIAV | |
| | GISGAIQHLAGMKDSKTIVAINKDPEAPIFQVADYGIVADLFKVVPE | |
| | MTEILKKK | |
| 87 | MAELRVLVAVKRVIDYAVKIRVKPDRTGVVTDGVKHSMNPFCEIA | ETFB |
| | VEEAVRLKEKKLVKEVIAVSCGPAQCQETIRTALAMGADRGIHVEV | |
| | PPAEAERLGPLQVARVLAKLAEKEKVDLVLLGKQAIDDDCNQTGQ | |
| | MTAGFLDWPQGTFASQVTLEGDKLKVEREIDGGLETLRLKLPAVVT | |
| | ADLRLNEPRYATLPNIMKAKKKKIEVIKPGDLGVDLTSKLSVISVED | |
| | PPQRTAGVKVETTEDLVAKLKEIGRI | |
| 88 | MLVPLAKLSCLAYQCFHALKIKKNYLPLCATRWSSTSTVPRITTHYT | ETFDH |
| | IYPRDKDKRWEGVNMERFAEEADVVIVGAGPAGLSAAVRLKQLAV | |
| | AHEKDIRVCLVEKAAQIGAHTLSGACLDPGAFKELFPDWKEKGAPL | |
| | NTPVTEDRFGILTEKYRIPVPILPGLPMNNHGNYIVRLGHLVSWMGE | |
| | QAEALGVEVYPGYAAAEVLFHDDGSVKGIATNDVGIQKDGAPKAT | |
| | FERGLELHAKVTIFAEGCHGHLAKQLYKKFDLRANCEPQTYGIGLK | |
| | ELWVIDEKNWKPGRVDHTVGWPLDRHTYGGSFLYHLNEGEPLVAL | |
| | GLVVGLDYQNPYLSPFREFQRWKHHPSIRPTLEGGKRIAYGARALN | |
| | EGGFQSIPKLTFPGGLLIGCSPGFMNVPKIKGTHTAMKSGILAAESIF | |
| | NQLTSENLQSKTIGLHVTEYEDNLKNSWVWKELYSVRNIRPSCHGV | |
| | LGVYGGMIYTGIFYWILRGMEPWTLKHKGSDFERLKPAKDCTPIEY | |
| | PKPDGQISFDLLSSVALSGTNHEHDQPAHLTLRDDSIPVNRNLSIYDG | |
| | PEQRFCPAGVYEFVPVEQGDGFRLQINAQNCVHCKTCDIKDPSQNIN | |
| | WVVPEGGGGPAYNGM | |
| 89 | MASESGKLWGGRFVGAVDPIMEKFNASIAYDRHLWEVDVQGSKA | ASL |
| | YSRGLEKAGLLTKAEMDQILHGLDKVAEEWAQGTFKLNSNDEDIH | |
| | TANERRLKELIGATAGKLHTGRSRNDQVVTDLRLWMRQTCSTLSG | |
| | LLWELIRTMVDRAEAERDVLFPGYTHLQRAQPIRWSHWILSHAVAL | |
| | TRDSERLLEVRKRINVLPLGSGAIAGNPLGVDRELLRAELNFGAITL | |
| | NSMDATSERDFVAEFLFWASLCMTHLSRMAEDLILYCTKEFSFVQL | |
| | SDAYSTGSSLMPQKKNPDSLELIRSKAGRVFGRCAGLLMTLKGLPS | |
| | TYNKDLQEDKEAVFEVSDTMSAVLQVATGVISTLQIHQENMGQAL | |
| | SPDMLATDLAYYLVRKGMPFRQAHEASGKAVFMAETKGVALNQL | |
| | SLQELQTISPLFSGDVICVWDYGHSVEQYGALGGTARSSVDWQIRQ | |
| | VRALLQAQQA | |
| 90 | MVGGSVPVFDEIILSTARMNRVLSFHSVSGILVCQAGCVLEELSRYV | D2HGDH |
| | EERDFIMPLDLGAKGSCHIGGNVATNAGGLRFLRYGSLHGTVLGLE | |
| | VVLADGTVLDCLTSLRKDNTGYDLKQLFIGSEGTLGIITTVSILCPPK | |
| | PRAVNVAFLGCPGFAEVLQTFSTCKGMLGEILSAFEFMDAVCMQLV | |
| | GRHLHLASPVQESPFYVLIETSGSNAGHDAEKLGHFLEHALGSGLVT | |
| | DGTMATDQRKVKMLWALRERITEALSRDGYVYKYDLSLPVERLYD | |
| | IVTDLRARLGPHAKHVVGYGHLGDGNLHLNVTAEAFSPSLLAALEP | |
| | HVYEWTAGQQGSVSAEHGVGFRKRDVLGYSKPPGALQLMQQLKA | |
| | LLDPKGILNPYKTLPSQA | |
| 91 | MAAMRKALPRRLVGLASLRAVSTSSMGTLPKRVKIVEVGPRDGLQ | HMGCL |
| | NEKNIVSTPVKIKLIDMLSEAGLSVIETTSFVSPKWVPQMGDHTEVL | |
| | KGIQKFPGINYPVLTPNLKGFEAAVAAGAKEVVIFGAASELFTKKNI | |
| | NCSIEESFQRFDAILKAAQSANISVRGYVSCALGCPYEGKISPAKVAE | |
| | VTKKFYSMGCYEISLGDTIGVGTPGIMKDMLSAVMQEVPLAALAV | |
| | HCHDTYGQALANTLMALQMGVSVVDSSVAGLGGCPYAQGASGNL | |
| | ATEDLVYMLEGLGIHTGVNLQKLLEAGNFICQALNRKTSSKVAQAT | |
| | CKL | |
| 92 | MAAASAVSVLLVAAERNRWHRLPSLLLPPRTWVWRQRTMKYTTA | MCCC1 |
| | TGRNITKVLIANRGEIACRVMRTAKKLGVQTVAVYSEADRNSMHV | |
| | DMADEAYSIGPAPSQQSYLSMEKIIQVAKTSAAQAIHPGCGFLSENM | |
| | EFAELCKQEGIIFIGPPPSAIRDMGIKSTSKSIMAAAGVPVVEGYHGE | |
| | DQSDQCLKEHARRIGYPVMIKAVRGGGGKGMRIVRSEQEFQEQLES | |
| | ARREAKKSFNDDAMLIEKFVDTPRHVEVQVFGDHHGNAVYLFERD | |
| | CSVQRRHQKIIEEAPAPGIKSEVRKKLGEAAVRAAKAVNYVGAGTV | |
| | EFIMDSKHNFCFMEMNTRLQVEHPVTEMITGTDLVEWQLRIAAGEK | |
| | IPLSQEEITLQGHAFEARIYAEDPSNNFMPVAGPLVHLSTPRADPSTR | |
| | IETGVRQGDEVSVHYDPMIAKLVVWAADRQAALTKLRYSLRQYNI | |
| | VGLHTNIDFLLNLSGHPEFEAGNVHTDFIPQHHKQLLLSRKAAAKES | |
| | LCQAALGLILKEKAMTDTFTLQAHDQFSPFSSSSGRRLNISYTRNMT | |
| | LKDGKNNVAIAVTYNHDGSYSMQIEDKTFQVLGNLYSEGDCTYLK | |
| | CSVNGVASKAKLIILENTIYLFSKEGSIEIDIPVPKYLSSVSSQETQGG | |
| | PLAPMTGTIEKVFVKAGDKVKAGDSLMVMIAMKMEHTIKSPKDGT | |
| | VKKVFYREGAQANRHTPLVEFEEEESDKRESE | |
| 93 | MWAVLRLALRPCARASPAGPRAYHGDSVASLGTQPDLGSALYQEN | MCCC2 |
| | YKQMKALVNQLHERVEHIKLGGGEKARALHISRGKLLPRERIDNLI | |
| | DPGSPFLELSQFAGYQLYDNEEVPGGGIITGIGRVSGVECMIIANDAT | |
| | VKGGAYYPVTVKKQLRAQEIAMQNRLPCIYLVDSGGAYLPRQADV | |
| | FPDRDHFGRTFYNQAIMSSKNIAQIAVVMGSCTAGGAYVPAMADE | |
| | NIIVRKQGTIFLAGPPLVKAATGEEVSAEDLGGADLHCRKSGVSDH | |
| | WALDDHHALHLTRKVVRNLNYQKKLDVTIEPSEEPLFPADELYGIV | |
| | GANLKRSFDVREVIARIVDGSRFTEFKAFYGDTLVTGFARIFGYPVGI | |
| | VGNNGVLFSESAKKGTHFVQLCCQRNIPLLFLQNITGFMVGREYEA | |
| | EGIAKDGAKMVAAVACAQVPKITLIIGGSYGAGNYGMCGRAYSPR | |
| | FLYIWPNARISVMGGEQAANVLATITKDQRAREGKQFSSADEAALK | |
| | EPIIKKFEEEGNPYYSSARVWDDGIIDPADTRLVLGLSFSAALNAPIE | |
| | KTDFGIFRM | |
| 94 | MAVAGPAPGAGARPRLDLQFLQRFLQILKVLFPSWSSQNALMFLTL | ABCD4 |
| | LCLTLLEQFVIYQVGLIPSQYYGVLGNKDLEGFKTLTFLAVMLIVLN | |
| | STLKSFDQFTCNLLYVSWRKDLTEHLHRLYFRGRAYYTLNVLRDDI | |
| | DNPDQRISQDVERFCRQLSSMASKLIISPFTLVYYTYQCFQSTGWLG | |
| | PVSIFGYFILGTVVNKTLMGPIVMKLVHQEKLEGDFRFKHMQIRVN | |
| | AEPAAFYRAGHVEHMRTDRRLQRLLQTQRELMSKELWLYIGINTFD | |
| | YLGSILSYVVIAIPIFSGVYGDLSPAELSTLVSKNAFVCIYLISCFTQLI | |
| | DLSTTLSDVAGYTHRIGQLRETLLDMSLKSQDCEILGESEWGLDTPP | |
| | GWPAAEPADTAFLLERVSISAPSSDKPLIKDLSLKISEGQSLLITGNTG | |
| | TGKTSLLRVLGGLWTSTRGSVQMLTDFGPHGVLFLPQKPFFTDGTL | |
| | REQVIYPLKEVYPDSGSADDERILRFLELAGLSNLVARTEGLDQQVD | |
| | WNWYDVLSPGEMQRLSFARLFYLQPKYAVLDEATSALTEEVESEL | |
| | YRIGQQLGMTFISVGHRQSLEKFHSLVLKLCGGGRWELMRIKVE | |
| 95 | MASAVSPANLPAVLLQPRWKRVVGWSGPVPRPRHGHRAVAIKELI | HCFC1 |
| | VVFGGGNEGIVDELHVYNTATNQWFIPAVRGDIPPGCAAYGFVCDG | |
| | TRLLVFGGMVEYGKYSNDLYELQASRWEWKRLKAKTPKNGPPPCP | |
| | RLGHSFSLVGNKCYLFGGLANDSEDPKNNIPRYLNDLYILELRPGSG | |
| | VVAWDIPITYGVLPPPRESHTAVVYTEKDNKKSKLVIYGGMSGCRL | |
| | GDLWTLDIDTLTWNKPSLSGVAPLPRSLHSATTIGNKMYVFGGWVP | |
| | LVMDDVKVATHEKEWKCTNTLACLNLDTMAWETILMDTLEDNIPR | |
| | ARAGHCAVAINTRLYIWSGRDGYRKAWNNQVCCKDLWYLETEKP | |
| | PPPARVQLVRANTNSLEVSWGAVATADSYLLQLQKYDIPATAATAT | |
| | SPTPNPVPSVPANPPKSPAPAAAAPAVQPLTQVGITLLPQAAPAPPTT | |
| | TTIQVLPTVPGSSISVPTAARTQGVPAVLKVTGPQATTGTPLVTMRP | |
| | ASQAGKAPVTVTSLPAGVRMVVPTQSAQGTVIGSSPQMSGMAALA | |
| | AAAAATQKIPPSSAPTVLSVPAGTTIVKTMAVTPGTTTLPATVKVAS | |
| | SPVMVSNPATRMLKTAAAQVGTSVSSATNTSTRPIITVHKSGTVTV | |
| | AQQAQVVTTVVGGVTKTITLVKSPISVPGGSALISNLGKVMSVVQT | |
| | KPVQTSAVTGQASTGPVTQIIQTKGPLPAGTILKLVTSADGKPTTIITT | |
| | TQASGAGTKPTILGISSVSPSTTKPGTTTIIKTIPMSAIITQAGATGVTS | |
| | SPGIKSPITIITTKVMTSGTGAPAKIITAVPKIATGHGQQGVTQVVLK | |
| | GAPGQPGTILRTVPMGGVRLVTPVTVSAVKPAVTTLVVKGTTGVTT | |
| | LGTVTGTVSTSLAGAGGHSTSASLATPITTLGTIATLSSQVINPTAITV | |
| | SAAQTTLTAAGGLTTPTITMQPVSQPTQVTLITAPSGVEAQPVHDLP | |
| | VSILASPTTEQPTATVTIADSGQGDVQPGTVTLVCSNPPCETHETGTT | |
| | NTATTTVVANLGGHPQPTQVQFVCDRQEAAASLVTSTVGQQNGSV | |
| | VRVCSNPPCETHETGTTNTATTATSNMAGQHGCSNPPCETHETGTT | |
| | NTATTAMSSVGANHQRDARRACAAGTPAVIRISVATGALEAAQGS | |
| | KSQCQTRQTSATSTTMTVMATGAPCSAGPLLGPSMAREPGGRSPAF | |
| | VQLAPLSSKVRLSSPSIKDLPAGRHSHAVSTAAMTRSSVGAGEPRM | |
| | APVCESLQGGSPSTTVTVTALEALLCPSATVTQVCSNPPCETHETGT | |
| | TNTATTSNAGSAQRVCSNPPCETHETGTTHTATTATSNGGTGQPEG | |
| | GQQPPAGRPCETHQTTSTGTTMSVSVGALLPDATSSHRTVESGLEV | |
| | AAAPSVTPQAGTALLAPFPTQRVCSNPPCETHETGTTHTATTVTSN | |
| | MSSNQDPPPAASDQGEVESTQGDSVNITSSSAITTTVSSTLTRAVTTV | |
| | TQSTPVPGPSVPPPEELQVSPGPRQQLPPRQLLQSASTALMGESAEV | |
| | LSASQTPELPAAVDLSSTGEPSSGQESAGSAVVATVVVQPPPPTQSE | |
| | VDQLSLPQELMAEAQAGTTTLMVTGLTPEELAVTAAAEAAAQAAA | |
| | TEEAQALAIQAVLQAAQQAVMGTGEPMDTSEAAATVTQAELGHLS | |
| | AEGQEGQATTIPIVLTQQELAALVQQQQLQEAQAQQQHHHLPTEAL | |
| | APADSLNDPAIESNCLNELAGTVPSTVALLPSTATESLAPSNTFVAPQ | |
| | PVVVASPAKLQAAATLTEVANGIESLGVKPDLPPPPSKAPMKKENQ | |
| | WFDVGVIKGTNVMVTHYFLPPDDAVPSDDDLGTVPDYNQLKKQEL | |
| | QPGTAYKFRVAGINACGRGPFSEISAFKTCLPGFPGAPCAIKISKSPD | |
| | GAHLTWEPPSVTSGKIIEYSVYLAIQSSQAGGELKSSTPAQLAFMRV | |
| | YCGPSPSCLVQSSSLSNAHIDYTTKPAIIFRIAARNEKGYGPATQVRW | |
| | LQETSKDSSGTKPANKRPMSSPEMKSAPKKSKADGQ | |
| 96 | MATSGAASAELVIGWCIFGLLLLAILAFCWIYVRKYQSRRESEVVST | LMBRD1 |
| | ITAIFSLAIALITSALLPVDIFLVSYMKNQNGTFKDWANANVSRQIED | |
| | TVLYGYYTLYSVILFCVFFWIPFVYFYYEEKDDDDTSKCTQIKTALK | |
| | YTLGFVVICALLLLVGAFVPLNVPNNKNSTEWEKVKSLFEELGSSH | |
| | GLAALSFSISSLTLIGMLAAITYTAYGMSALPLNLIKGTRSAAYERLE | |
| | NTEDIEEVEQHIQTIKSKSKDGRPLPARDKRALKQFEERLRTLKKRE | |
| | RHLEFIENSWWTKFCGALRPLKIVWGIFFILVALLFVISLFLSNLDKA | |
| | LHSAGIDSGFIIFGANLSNPLNMLLPLLQTVFPLDYILITIIIMYFIFTSM | |
| | AGIRNIGIWFFWIRLYKIRRGRTRPQALLFLCMILLLIVLHTSYMIYSL | |
| | APQYVMYGSQNYLIETNITSDNHKGNSTLSVPKRCDADAPEDQCTV | |
| | TRTYLFLHKFWFFSAAYYFGNWAFLGVFLIGLIVSCCKGKKSVIEGV | |
| | DEDSDISDDEPSVYSA | |
| 97 | MSAKSRTIGIIGAPFSKGQPRGGVEEGPTVLRKAGLLEKLKEQECDV | ARG1 |
| | KDYGDLPFADIPNDSPFQIVKNPRSVGKASEQLAGKVAEVKKNGRIS | |
| | LVLGGDHSLAIGSISGHARVHPDLGVIWVDAHTDINTPLTTTSGNLH | |
| | GQPVSFLLKELKGKIPDVPGFSWVTPCISAKDIVYIGLRDVDPGEHYI | |
| | LKTLGIKYFSMTEVDRLGIGKVMEETLSYLLGRKKRPIHLSFDVDGL | |
| | DPSFTPATGTPVVGGLTYREGLYITEEIYKTGLLSGLDIMEVNPSLGK | |
| | TPEEVTRTVNTAVAITLACFGLAREGNHKPIDYLNPPK | |
| 98 | MKSNPAIQAAIDLTAGAAGGTACVLTGQPFDTMKVKMQTFPDLYR | SLC25A15 |
| | GLTDCCLKTYSQVGFRGFYKGTSPALIANIAENSVLFMCYGFCQQV | |
| | VRKVAGLDKQAKLSDLQNAAAGSFASAFAALVLCPTELVKCRLQT | |
| | MYEMETSGKIAKSQNTVWSVIKSILRKDGPLGFYHGLSSTLLREVPG | |
| | YFFFFGGYELSRSFFASGRSKDELGPVPLMLSGGVGGICLWLAVYPV | |
| | DCIKSRIQVLSMSGKQAGFIRTFINVVKNEGITALYSGLKPTMIRAFP | |
| | ANGALFLAYEYSRKLMMNQLEAY | |
| 99 | MAAAKVALTKRADPAELRTIFLKYASIEKNGEFFMSPNDFVTRYLNI | SLC25A13 |
| | FGESQPNPKTVELLSGVVDQTKDGLISFQEFVAFESVLCAPDALFMV | |
| | AFQLFDKAGKGEVTFEDVKQVFGQTTIHQHIPFNWDSEFVQLHFGK | |
| | ERKRHLTYAEFTQFLLEIQLEHAKQAFVQRDNARTGRVTAIDFRDI | |
| | MVTIRPHVLTPFVEECLVAAAGGTTSHQVSFSYFNGFNSLLNNMELI | |
| | RKIYSTLAGTRKDVEVTKEEFVLAAQKFGQVTPMEVDILFQLADLY | |
| | EPRGRMTLADIERIAPLEEGTLPFNLAEAQRQKASGDSARPVLLQVA | |
| | ESAYRFGLGSVAGAVGATAVYPIDLVKTRMQNQRSTGSFVGELMY | |
| | KNSFDCFKKVLRYEGFFGLYRGLLPQLLGVAPEKAIKLTVNDFVRD | |
| | KFMHKDGSVPLAAEILAGGCAGGSQVIFTNPLEIVKIRLQVAGEITT | |
| | GPRVSALSVVRDLGFFGIYKGAKACFLRDIPFSAIYFPCYAHVKASF | |
| | ANEDGQVSPGSLLLAGAIAGMPAASLVTPADVIKTRLQVAARAGQT | |
| | TYSGVIDCFRKILREEGPKALWKGAGARVFRSSPQFGVTLLTYELLQ | |
| | RWFYIDFGGVKPMGSEPVPKSRINLPAPNPDHVGGYKLAVATFAGI | |
| | ENKFGLYLPLFKPSVSTSKAIGGGP | |
| 100 | MQPQSVLHSGYFHPLLRAWQTATTTLNASNLIYPIFVTDVPDDIQPIT | ALAD |
| | SLPGVARYGVKRLEEMLRPLVEEGLRCVLIFGVPSRVPKDERGSAA | |
| | DSEESPAIEAIHLLRKTFPNLLVACDVCLCPYTSHGHCGLLSENGAF | |
| | RAEESRQRLAEVALAYAKAGCQVVAPSDMMDGRVEAIKEALMAH | |
| | GLGNRVSVMSYSAKFASCFYGPFRDAAKSSPAFGDRRCYQLPPGAR | |
| | GLALRAVDRDVREGADMLMVKPGMPYLDIVREVKDKHPDLPLAV | |
| | YHVSGEFAMLWHGAQAGAFDLKAAVLEAMTAFRRAGADIIITYYT | |
| | PQLLQWLKEE | |
| 101 | MALQLGRLSSGPCWLVARGGCGGPRAWSQCGGGGLRAWSQRSAA | CPOX |
| | GRVCRPPGPAGTEQSRGLGHGSTSRGGPWVGTGLAAALAGLVGLA | |
| | TAAFGHVQRAEMLPKTSGTRATSLGRPEEEEDELAHRCSSFMAPPV | |
| | TDLGELRRRPGDMKTKMELLILETQAQVCQALAQVDGGANFSVDR | |
| | WERKEGGGGISCVLQDGCVFEKAGVSISVVHGNLSEEAAKQMRSR | |
| | GKVLKTKDGKLPFCAMGVSSVIHPKNPHAPTIHFNYRYFEVEEADG | |
| | NKQWWFGGGCDLTPTYLNQEDAVHFHRTLKEACDQHGPDLYPKF | |
| | KKWCDDYFFIAHRGERRGIGGIFFDDLDSPSKEEVFRFVQSCARAVV | |
| | PSYIPLVKKHCDDSFTPQEKLWQQLRRGRYVEFNLLYDRGTKFGLF | |
| | TPGSRIESILMSLPLTARWEYMHSPSENSKEAEILEVLRHPRDWVR | |
| 102 | MSGNGNAAATAEENSPKMRVIRVGTRKSQLARIQTDSVVATLKAS | HMBS |
| | YPGLQFEIIAMSTTGDKILDTALSKIGEKSLFTKELEHALEKNEVDLV | |
| | VHSLKDLPTVLPPGFTIGAICKRENPHDAVVFHPKFVGKTLETLPEK | |
| | SVVGTSSLRRAAQLQRKFPHLEFRSIRGNLNTRLRKLDEQQEFSAIIL | |
| | ATAGLQRMGWHNRVGQILHPEECMYAVGQGALGVEVRAKDQDIL | |
| | DLVGVLHDPETLLRCIAERAFLRHLEGGCSVPVAVHTAMKDGQLY | |
| | LTGGVWSLDGSDSIQETMQATIHVPAQHEDGPEDDPQLVGITARNIP | |
| | RGPQLAAQNLGISLANLLLSKGAKNILDVARQLNDAH | |
| 103 | MGRTVVVLGGGISGLAASYHLSRAPCPPKVVLVESSERLGGWIRSV | PPOX |
| | RGPNGAIFELGPRGIRPAGALGARTLLLVSELGLDSEVLPVRGDHPA | |
| | AQNRFLYVGGALHALPTGLRGLLRPSPPFSKPLFWAGLRELTKPRG | |
| | KEPDETVHSFAQRRLGPEVASLAMDSLCRGVFAGNSRELSIRSCFPS | |
| | LFQAEQTHRSILLGLLLGAGRTPQPDSALIRQALAERWSQWSLRGG | |
| | LEMLPQALETHLTSRGVSVLRGQPVCGLSLQAEGRWKVSLRDSSLE | |
| | ADHVISAIPASVLSELLPAEAAPLARALSAITAVSVAVVNLQYQGAH | |
| | LPVQGFGHLVPSSEDPGVLGIVYDSVAFPEQDGSPPGLRVTVMLGG | |
| | SWLQTLEASGCVLSQELFQQRAQEAAATQLGLKEMPSHCLVHLHK | |
| | NCIPQYTLGHWQKLESARQFLTAHRLPLTLAGASYEGVAVNDCIES | |
| | GRQAAVSVLGTEPNS | |
| 104 | MAHAHIQGGRRAKSRFVVCIMSGARSKLALFLCGCYVVALGAHTG | BTD |
| | EESVADHHEAEYYVAAVYEHPSILSLNPLALISRQEALELMNQNLDI | |
| | YEQQVMTAAQKDVQIIVFPEDGIHGFNFTRTSIYPFLDFMPSPQVVR | |
| | WNPCLEPHRFNDTEVLQRLSCMAIRGDMFLVANLGTKEPCHSSDPR | |
| | CPKDGRYQFNTNVVFSNNGTLVDRYRKHNLYFEAAFDVPLKVDLIT | |
| | FDTPFAGRFGIFTCFDILFFDPAIRVLRDYKVKHVVYPTAWMNQLPL | |
| | LAAIEIQKAFAVAFGINVLAANVHHPVLGMTGSGIHTPLESFWYHD | |
| | MENPKSHLIIAQVAKNPVGLIGAENATGETDPSHSKFLKILSGDPYC | |
| | EKDAQEVHCDEATKWNVNAPPTFHSEMMYDNFTLVPVWGKEGYL | |
| | HVCSNGLCCYLLYERPTLSKELYALGVFDGLHTVHGTYYIQVCALV | |
| | RCGGLGFDTCGQEITEATGIFEFHLWGNFSTSYIFPLFLTSGMTLEVP | |
| | DQLGWENDHYFLRKSRLSSGLVTAALYGRLYERD | |
| 105 | MEDRLHMDNGLVPQKIVSVHLQDSTLKEVKDQVSNKQAQILEPKP | HLCS |
| | EPSLEIKPEQDGMEHVGRDDPKALGEEPKQRRGSASGSEPAGDSDR | |
| | GGGPVEHYHLHLSSCHECLELENSTIESVKFASAENIPDLPYDYSSSL | |
| | ESVADETSPEREGRRVNLTGKAPNILLYVGSDSQEALGRFHEVRSVL | |
| | ADCVDIDSYILYHLLEDSALRDPWTDNCLLLVIATRESIPEDLYQKF | |
| | MAYLSQGGKVLGLSSSFTFGGFQVTSKGALHKTVQNLVFSKADQSE | |
| | VKLSVLSSGCRYQEGPVRLSPGRLQGHLENEDKDRMIVHVPFGTRG | |
| | GEAVLCQVHLELPPSSNIVQTPEDFNLLKSSNFRRYEVLREILTTLGL | |
| | SCDMKQVPALTPLYLLSAAEEIRDPLMQWLGKHVDSEGEIKSGQLS | |
| | LRFVSSYVSEVEITPSCIPVVTNMEAFSSEHFNLEIYRQNLQTKQLGK | |
| | VILFAEVTPTTMRLLDGLMFQTPQEMGLIVIAARQTEGKGRGGNVW | |
| | LSPVGCALSTLLISIPLRSQLGQRIPFVQHLMSVAVVEAVRSIPEYQDI | |
| | NLRVKWPNDIYYSDLMKIGGVLVNSTLMGETFYILIGCGFNVTNSN | |
| | PTICINDLITEYNKQHKAELKPLRADYLIARVVTVLEKLIKEFQDKGP | |
| | NSVLPLYYRYWVHSGQQVHLGSAEGPKVSIVGLDDSGFLQVHQEG | |
| | GEVVTVHPDGNSFDMLRNLILPKRR | |
| 106 | MLKFRTVHGGLRLLGIRRTSTAPAASPNVRRLEYKPIKKVMVANRG | PC |
| | EIAIRVFRACTELGIRTVAIYSEQDTGQMHRQKADEAYLIGRGLAPV | |
| | QAYLHIPDIIKVAKENNVDAVHPGYGFLSERADFAQACQDAGVRFI | |
| | GPSPEVVRKMGDKVEARAIAIAAGVPVVPGTDAPITSLHEAHEFSNT | |
| | YGFPIIFKAAYGGGGRGMRVVHSYEELEENYTRAYSEALAAFGNGA | |
| | LFVEKFIEKPRHIEVQILGDQYGNILHLYERDCSIQRRHQKVVEIAPA | |
| | AHLDPQLRTRLTSDSVKLAKQVGYENAGTVEFLVDRHGKHYFIEV | |
| | NSRLQVEHTVTEEITDVDLVHAQIHVAEGRSLPDLGLRQENIRINGC | |
| | AIQCRVTTEDPARSFQPDTGRIEVFRSGEGMGIRLDNASAFQGAVISP | |
| | HYDSLLVKVIAHGKDHPTAATKMSRALAEFRVRGVKTNIAFLQNV | |
| | LNNQQFLAGTVDTQFIDENPELFQLRPAQNRAQKLLHYLGHVMVN | |
| | GPTTPIPVKASPSPTDPVVPAVPIGPPPAGFRDILLREGPEGFARAVRN | |
| | HPGLLLMDTTFRDAHQSLLATRVRTHDLKKIAPYVAHNFSKLFSME | |
| | NWGGATFDVAMRFLYECPWRRLQELRELIPNIPFQMLLRGANAVG | |
| | YTNYPDNVVFKFCEVAKENGMDVFRVFDSLNYLPNMLLGMEAAG | |
| | SAGGVVEAAISYTGDVADPSRTKYSLQYYMGLAEELVRAGTHILCI | |
| | KDMAGLLKPTACTMLVSSLRDRFPDLPLHIHTHDTSGAGVAAMLA | |
| | CAQAGADVVDVAADSMSGMTSQPSMGALVACTRGTPLDTEVPME | |
| | RVFDYSEYWEGARGLYAAFDCTATMKSGNSDVYENEIPGGQYTNL | |
| | HFQAHSMGLGSKFKEVKKAYVEANQMLGDLIKVTPSSKIVGDLAQ | |
| | FMVQNGLSRAEAEAQAEELSFPRSVVEFLQGYIGVPHGGFPEPFRSK | |
| | VLKDLPRVEGRPGASLPPLDLQALEKELVDRHGEEVTPEDVLSAAM | |
| | YPDVFAHFKDFTATFGPLDSLNTRLFLQGPKIAEEFEVELERGKTLHI | |
| | KALAVSDLNRAGQRQVFFELNGQLRSILVKDTQAMKEMHFHPKAL | |
| | KDVKGQIGAPMPGKVIDIKVVAGAKVAKGQPLCVLSAMKMETVVT | |
| | SPMEGTVRKVHVTKDMTLEGDDLILEIE | |
| 107 | MVDSTEYEVASQPEVETSPLGDGASPGPEQVKLKKEISLLNGVCLIV | SLC7A7 |
| | GNMIGSGIFVSPKGVLIYSASFGLSLVIWAVGGLFSVFGALCYAELG | |
| | TTIKKSGASYAYILEAFGGFLAFIRLWTSLLIIEPTSQAIIAITFANYMV | |
| | QPLFPSCFAPYAASRLLAAACICLLTFINCAYVKWGTLVQDIFTYAK | |
| | VLALIAVIVAGIVRLGQGASTHFENSFEGSSFAVGDIALALYSALFSY | |
| | SGWDTLNYVTEEIKNPERNLPLSIGISMPIVTIIYILTNVAYYTVLDM | |
| | RDILASDAVAVTFADQIFGIFNWIIPLSVALSCFGGLNASIVAASRLFF | |
| | VGSREGHLPDAICMIHVERFTPVPSLLFNGIMALIYLCVEDIFQLINY | |
| | YSFSYWFFVGLSIVGQLYLRWKEPDRPRPLKLSVFFPIVFCLCTIFLV | |
| | AVPLYSDTINSLIGIAIALSGLPFYFLIIRVPEHKRPLYLRRIVGSATRY | |
| | LQVLCMSVAAEMDLEDGGEMPKQRDPKSN | |
| 108 | MVPRLLLRAWPRGPAVGPGAPSRPLSAGSGPGQYLQRSIVPTMHYQ | CPT2 |
| | DSLPRLPIPKLEDTIRRYLSAQKPLLNDGQFRKTEQFCKSFENGIGKE | |
| | LHEQLVALDKQNKHTSYISGPWFDMYLSARDSVVLNFNPFMAFNP | |
| | DPKSEYNDQLTRATNMTVSAIRFLKTLRAGLLEPEVFHLNPAKSDTI | |
| | TFKRLIRFVPSSLSWYGAYLVNAYPLDMSQYFRLFNSTRLPKPSRDE | |
| | LFTDDKARHLLVLRKGNFYIFDVLDQDGNIVSPSEIQAHLKYILSDSS | |
| | PAPEFPLAYLTSENRDIWAELRQKLMSSGNEESLRKVDSAVFCLCLD | |
| | DFPIKDLVHLSHNMLHGDGTNRWFDKSFNLIIAKDGSTAVHFEHSW | |
| | GDGVAVLRFFNEVFKDSTQTPAVTPQSQPATTDSTVTVQKLNFELT | |
| | DALKTGITAAKEKFDATMKTLTIDCVQFQRGGKEFLKKQKLSPDAV | |
| | AQLAFQMAFLRQYGQTVATYESCSTAAFKHGRTETIRPASVYTKRC | |
| | SEAFVREPSRHSAGELQQMMVECSKYHGQLTKEAAMGQGFDRHLF | |
| | ALRHLAAAKGIILPELYLDPAYGQINHNVLSTSTLSSPAVNLGGFAP | |
| | VVSDGFGVGYAVHDNWIGCNVSSYPGRNAREFLQCVEKALEDMFD | |
| | ALEGKSIKS | |
| 109 | MAAGFGRCCRVLRSISRFHWRSQHTKANRQREPGLGFSFEFTEQQK | ACADM |
| | EFQATARKFAREEIIPVAAEYDKTGEYPVPLIRRAWELGLMNTHIPE | |
| | NCGGLGLGTFDACLISEELAYGCTGVQTAIEGNSLGQMPIIIAGNDQ | |
| | QKKKYLGRMTEEPLMCAYCVTEPGAGSDVAGIKTKAEKKGDEYII | |
| | NGQKMWITNGGKANWYFLLARSDPDPKAPANKAFTGFIVEADTPG | |
| | IQIGRKELNMGQRCSDTRGIVFEDVKVPKENVLIGDGAGFKVAMGA | |
| | FDKTRPVVAAGAVGLAQRALDEATKYALERKTFGKLLVEHQAISF | |
| | MLAEMAMKVELARMSYQRAAWEVDSGRRNTYYASIAKAFAGDIA | |
| | NQLATDAVQILGGNGFNTEYPVEKLMRDAKIYQIYEGTSQIQRLIVA | |
| | REHIDKYKN | |
| 110 | MAAALLARASGPARRALCPRAWRQLHTIYQSVELPETHQMLLQTC | ACADS |
| | RDFAEKELFPIAAQVDKEHLFPAAQVKKMGGLGLLAMDVPEELGG | |
| | AGLDYLAYAIAMEEISRGCASTGVIMSVNNSLYLGPILKFGSKEQKQ | |
| | AWVTPFTSGDKIGCFALSEPGNGSDAGAASTTARAEGDSWVLNGT | |
| | KAWITNAWEASAAVVFASTDRALQNKGISAFLVPMPTPGLTLGKKE | |
| | DKLGIRGSSTANLIFEDCRIPKDSILGEPGMGFKIAMQTLDMGRIGIA | |
| | SQALGIAQTALDCAVNYAENRMAFGAPLTKLQVIQFKLADMALAL | |
| | ESARLLTWRAAMLKDNKKPFIKEAAMAKLAASEAATAISHQAIQIL | |
| | GGMGYVTEMPAERHYRDARITEIYEGTSEIQRLVIAGHLLRSYRS | |
| 111 | MQAARMAASLGRQLLRLGGGSSRLTALLGQPRPGPARRPYAGGAA | ACADVL |
| | QLALDKSDSHPSDALTRKKPAKAESKSFAVGMFKGQLTTDQVFPYP | |
| | SVLNEEQTQFLKELVEPVSRFFEEVNDPAKNDALEMVEETTWQGLK | |
| | ELGAFGLQVPSELGGVGLCNTQYARLVEIVGMHDLGVGITLGAHQS | |
| | IGFKGILLFGTKAQKEKYLPKLASGETVAAFCLTEPSSGSDAASIRTS | |
| | AVPSPCGKYYTLNGSKLWISNGGLADIFTVFAKTPVTDPATGAVKE | |
| | KITAFVVERGFGGITHGPPEKKMGIKASNTAEVFFDGVRVPSENVLG | |
| | EVGSGFKVAMHILNNGRFGMAAALAGTMRGIIAKAVDHATNRTQF | |
| | GEKIHNFGLIQEKLARMVMLQYVTESMAYMVSANMDQGATDFQIE | |
| | AAISKIFGSEAAWKVTDECIQIMGGMGFMKEPGVERVLRDLRIFRIF | |
| | EGTNDILRLFVALQGCMDKGKELSGLGSALKNPFGNAGLLLGEAG | |
| | KQLRRRAGLGSGLSLSGLVHPELSRSGELAVRALEQFATVVEAKLIK | |
| | HKKGIVNEQFLLQRLADGAIDLYAMVVVLSRASRSLSEGHPTAQHE | |
| | KMLCDTWCIEAAARIREGMAALQSDPWQQELYRNFKSISKALVER | |
| | GGVVTSNPLGF | |
| 112 | MGHSKQIRILLLNEMEKLEKTLFRLEQGYELQFRLGPTLQGKAVTV | AGL |
| | YTNYPFPGETFNREKFRSLDWENPTEREDDSDKYCKLNLQQSGSFQ | |
| | YYFLQGNEKSGGGYIVVDPILRVGADNHVLPLDCVTLQTFLAKCLG | |
| | PFDEWESRLRVAKESGYNMIHFTPLQTLGLSRSCYSLANQLELNPDF | |
| | SRPNRKYTWNDVGQLVEKLKKEWNVICITDVVYNHTAANSKWIQE | |
| | HPECAYNLVNSPHLKPAWVLDRALWRFSCDVAEGKYKEKGIPALIE | |
| | NDHHMNSIRKIIWEDIFPKLKLWEFFQVDVNKAVEQFRRLLTQENR | |
| | RVTKSDPNQHLTIIQDPEYRRFGCTVDMNIALTTFIPHDKGPAAIEEC | |
| | CNWFHKRMEELNSEKHRLINYHQEQAVNCLLGNVFYERLAGHGPK | |
| | LGPVTRKHPLVTRYFTFPFEEIDFSMEESMIHLPNKACFLMAHNGW | |
| | VMGDDPLRNFAEPGSEVYLRRELICWGDSVKLRYGNKPEDCPYLW | |
| | AHMKKYTEITATYFQGVRLDNCHSTPLHVAEYMLDAARNLQPNLY | |
| | VVAELFTGSEDLDNVFVTRLGISSLIREAMSAYNSHEEGRLVYRYG | |
| | GEPVGSFVQPCLRPLMPAIAHALFMDITHDNECPIVHRSAYDALPST | |
| | TIVSMACCASGSTRGYDELVPHQISVVSEERFYTKWNPEALPSNTGE | |
| | VNFQSGIIAARCAISKLHQELGAKGFIQVYVDQVDEDIVAVTRHSPSI | |
| | HQSVVAVSRTAFRNPKTSFYSKEVPQMCIPGKIEEVVLEARTIERNT | |
| | KPYRKDENSINGTPDITVEIREHIQLNESKIVKQAGVATKGPNEYIQEI | |
| | EFENLSPGSVIIFRVSLDPHAQVAVGILRNHLTQFSPHFKSGSLAVDN | |
| | ADPILKIPFASLASRLTLAELNQILYRCESEEKEDGGGCYDIPNWSAL | |
| | KYAGLQGLMSVLAEIRPKNDLGHPFCNNLRSGDWMIDYVSNRLISR | |
| | SGTIAEVGKWLQAMFFYLKQIPRYLIPCYFDAILIGAYTTLLDTAWK | |
| | QMSSFVQNGSTFVKHLSLGSVQLCGVGKFPSLPILSPALMDVPYRLN | |
| | EITKEKEQCCVSLAAGLPHFSSGIFRCWGRDTFIALRGILLITGRYVE | |
| | ARNIILAFAGTLRHGLIPNLLGEGIYARYNCRDAVWWWLQCIQDYC | |
| | KMVPNGLDILKCPVSRMYPTDDSAPLPAGTLDQPLFEVIQEAMQKH | |
| | MQGIQFRERNAGPQIDRNMKDEGFNITAGVDEETGFVYGGNRFNC | |
| | GTWMDKMGESDRARNRGIPATPRDGSAVEIVGLSKSAVRWLLELS | |
| | KKNIFPYHEVTVKRHGKAIKVSYDEWNRKIQDNFEKLFHVSEDPSD | |
| | LNEKHPNLVHKRGIYKDSYGASSPWCDYQLRPNFTIAMVVAPELFT | |
| | TEKAWKALEIAEKKLLGPLGMKTLDPDDMVYCGIYDNALDNDNY | |
| | NLAKGFNYHQGPEWLWPIGYFLRAKLYFSRLMGPETTAKTIVLVKN | |
| | VLSRHYVHLERSPWKGLPELTNENAQYCPFSCETQAWSIATILETLY | |
| | DL | |
| 113 | MEEGMNVLHDFGIQSTHYLQVNYQDSQDWFILVSVIADLRNAFYV | G6PC |
| | LFPIWFHLQEAVGIKLLWVAVIGDWLNLVFKWILFGQRPYWWVLD | |
| | TDYYSNTSVPLIKQFPVTCETGPGSPSGHAMGTAGVYYVMVTSTLSI | |
| | FQGKIKPTYRFRCLNVILWLGFWAVQLNVCLSRIYLAAHFPHQVVA | |
| | GVLSGIAVAETFSHIHSIYNASLKKYFLITFFLFSFAIGFYLLLKGLGV | |
| | DLLWTLEKAQRWCEQPEWVHIDTTPFASLLKNLGTLFGLGLALNSS | |
| | MYRESCKGKLSKWLPFRLSSIVASLVLLHVFDSLKPPSQVELVFYVL | |
| | SFCKSAVVPLASVSVIPYCLAQVLGQPHKKSL | |
| 114 | MAAPMTPAARPEDYEAALNAALADVPELARLLEIDPYLKPYAVDF | GBE1 |
| | QRRYKQFSQILKNIGENEGGIDKFSRGYESFGVHRCADGGLYCKEW | |
| | APGAEGVFLTGDFNGWNPFSYPYKKLDYGKWELYIPPKQNKSVLV | |
| | PHGSKLKVVITSKSGEILYRISPWAKYVVREGDNVNYDWIHWDPEH | |
| | SYEFKHSRPKKPRSLRIYESHVGISSHEGKVASYKHFTCNVLPRIKGL | |
| | GYNCIQLMAIMEHAYYASFGYQITSFFAASSRYGTPEELQELVDTAH | |
| | SMGIIVLLDVVHSHASKNSADGLNMFDGTDSCYFHSGPRGTHDLW | |
| | DSRLFAYSSWEILRFLLSNIRWWLEEYRFDGFRFDGVTSMLYHHHG | |
| | VGQGFSGDYSEYFGLQVDEDALTYLMLANHLVHTLCPDSITIAEDV | |
| | SGMPALCSPISQGGGGFDYRLAMAIPDKWIQLLKEFKDEDWNMGDI | |
| | VYTLTNRRYLEKCIAYAESHDQALVGDKSLAFWLMDAEMYTNMS | |
| | VLTPFTPVIDRGIQLHKMIRLITHGLGGEGYLNFMGNEFGHPEWLDF | |
| | PRKGNNESYHYARRQFHLTDDDLLRYKFLNNFDRDMNRLEERYG | |
| | WLAAPQAYVSEKHEGNKIIAFERAGLLFIFNFHPSKSYTDYRVGTAL | |
| | PGKFKIVLDSDAAEYGGHQRLDHSTDFFSEAFEHNGRPYSLLVYIPS | |
| | RVALILQNVDLPN | |
| 115 | MRSRSNSGVRLDGYARLVQQTILCHQNPVTGLLPASYDQKDAWVR | PHKA1 |
| | DNVYSILAVWGLGLAYRKNADRDEDKAKAYELEQSVVKLMRGLL | |
| | HCMIRQVDKVESFKYSQSTKDSLHAKYNTKTCATVVGDDQWGHL | |
| | QLDATSVYLLFLAQMTASGLHIIHSLDEVNFIQNLVFYIEAAYKTAD | |
| | FGIWERGDKTNQGISELNASSVGMAKAALEALDELDLFGVKGGPQS | |
| | VIHVLADEVQHCQSILNSLLPRASTSKEVDASLLSVVSFPAFAVEDS | |
| | QLVELTKQEIITKLQGRYGCCRFLRDGYKTPKEDPNRLYYEPAELKL | |
| | FENIECEWPLFWTYFILDGVFSGNAEQVQEYKEALEAVLIKGKNGV | |
| | PLLPELYSVPPDRVDEEYQNPHTVDRVPMGKLPHMWGQSLYILGSL | |
| | MAEGFLAPGEIDPLNRRFSTVPKPDVVVQVSILAETEEIKTILKDKGI | |
| | YVETIAEVYPIRVQPARILSHIYSSLGCNNRMKLSGRPYRHMGVLGT | |
| | SKLYDIRKTIFTFTPQFIDQQQFYLALDNKMIVEMLRTDLSYLCSRW | |
| | RMTGQPTITFPISHSMLDEDGTSLNSSILAALRKMQDGYFGGARVQT | |
| | GKLSEFLTTSCCTHLSFMDPGPEGKLYSEDYDDNYDYLESGNWMN | |
| | DYDSTSHARCGDEVARYLDHLLAHTAPHPKLAPTSQKGGLDRFQA | |
| | AVQTTCDLMSLVTKAKELHVQNVHMYLPTKLFQASRPSFNLLDSP | |
| | HPRQENQVPSVRVEIHLPRDQSGEVDFKALVLQLKETSSLQEQADIL | |
| | YMLYTMKGPDWNTELYNERSATVRELLTELYGKVGEIRHWGLIRYI | |
| | SGILRKKVEALDEACTDLLSHQKHLTVGLPPEPREKTISAPLPYEALT | |
| | QLIDEASEGDMSISILTQEIMVYLAMYMRTQPGLFAEMFRLRIGLIIQ | |
| | VMATELAHSLRCSAEEATEGLMNLSPSAMKNLLHHILSGKEFGVER | |
| | SVRPTDSNVSPAISIHEIGAVGATKTERTGIMQLKSEIKQVEFRRLSIS | |
| | AESQSPGTSMTPSSGSFPSAYDQQSSKDSRQGQWQRRRRLDGALNR | |
| | VPVGFYQKVWKVLQKCHGLSVEGFVLPSSTTREMTPGEIKFSVHVE | |
| | SVLNRVPQPEYRQLLVEAILVLTMLADIEIHSIGSIIAVEKIVHIANDL | |
| | FLQEQKTLGADDTMLAKDPASGICTLLYDSAPSGRFGTMTYLSKAA | |
| | ATYVQEFLPHSICAMQ | |
| 116 | MRSRSNSGVRLDGYARLVQQTILCYQNPVTGLLSASHEQKDAWVR | PHKA2 |
| | DNIYSILAVWGLGMAYRKNADRDEDKAKAYELEQNVVKLMRGLL | |
| | QCMMRQVAKVEKFKHTQSTKDSLHAKYNTATCGTVVGDDQWGH | |
| | LQVDATSLFLLFLAQMTASGLRIIFTLDEVAFIQNLVFYIEAAYKVA | |
| | DYGMWERGDKTNQGIPELNASSVGMAKAALEAIDELDLFGAHGGR | |
| | KSVIHVLPDEVEHCQSILFSMLPRASTSKEIDAGLLSIISFPAFAVEDV | |
| | NLVNVTKNEIISKLQGRYGCCRFLRDGYKTPREDPNRLHYDPAELK | |
| | LFENIECEWPVFWTYFIIDGVFSGDAVQVQEYREALEGILIRGKNGIR | |
| | LVPELYAVPPNKVDEEYKNPHTVDRVPMGKVPHLWGQSLYILSSLL | |
| | AEGFLAAGEIDPLNRRFSTSVKPDVVVQVTVLAENNHIKDLLRKHG | |
| | VNVQSIADIHPIQVQPGRILSHIYAKLGRNKNMNLSGRPYRHIGVLG | |
| | TSKLYVIRNQIFTFTPQFTDQHHFYLALDNEMIVEMLRIELAYLCTC | |
| | WRMTGRPTLTFPISRTMLTNDGSDIHSAVLSTIRKLEDGYFGGARVK | |
| | LGNLSEFLTTSFYTYLTFLDPDCDEKLFDNASEGTFSPDSDSDLVGY | |
| | LEDTCNQESQDELDHYINHLLQSTSLRSYLPPLCKNTEDRHVFSAIH | |
| | STRDILSVMAKAKGLEVPFVPMTLPTKVLSAHRKSLNLVDSPQPLLE | |
| | KVPESDFQWPRDDHGDVDCEKLVEQLKDCSNLQDQADILYILYVIK | |
| | GPSWDTNLSGQHGVTVQNLLGELYGKAGLNQEWGLIRYISGLLRK | |
| | KVEVLAEACTDLLSHQKQLTVGLPPEPREKIISAPLPPEELTKLIYEA | |
| | SGQDISIAVLTQEIVVYLAMYVRAQPSLFVEMLRLRIGLIIQVMATEL | |
| | ARSLNCSGEEASESLMNLSPFDMKNLLHHILSGKEFGVERSVRPIHS | |
| | STSSPTISIHEVGHTGVTKTERSGINRLRSEMKQMTRRFSADEQFFSV | |
| | GQAASSSAHSSKSARSSTPSSPTGTSSSDSGGHHIGWGERQGQWLRR | |
| | RRLDGAINRVPVGFYQRVWKILQKCHGLSIDGYVLPSSTTREMTPH | |
| | EIKFAVHVESVLNRVPQPEYRQLLVEAIMVLTLLSDTEMTSIGGIIHV | |
| | DQIVQMASQLFLQDQVSIGAMDTLEKDQATGICHFFYDSAPSGAYG | |
| | TMTYLTRAVASYLQELLPNSGCQMQ | |
| 117 | MAGAAGLTAEVSWKVLERRARTKRSGSVYEPLKSINLPRPDNETL | PHKB |
| | WDKLDHYYRIVKSTLLLYQSPTTGLFPTKTCGGDQKAKIQDSLYCA | |
| | AGAWALALAYRRIDDDKGRTHELEHSAIKCMRGILYCYMRQADKV | |
| | QQFKQDPRPTTCLHSVFNVHTGDELLSYEEYGHLQINAVSLYLLYL | |
| | VEMISSGLQIIYNTDEVSFIQNLVFCVERVYRVPDFGVWERGSKYNN | |
| | GSTELHSSSVGLAKAALEAINGFNLFGNQGCSWSVIFVDLDAHNRN | |
| | RQTLCSLLPRESRSHNTDAALLPCISYPAFALDDEVLFSQTLDKVVR | |
| | KLKGKYGFKRFLRDGYRTSLEDPNRCYYKPAEIKLFDGIECEFPIFFL | |
| | YMMIDGVFRGNPKQVQEYQDLLTPVLHHTTEGYPVVPKYYYVPAD | |
| | FVEYEKNNPGSQKRFPSNCGRDGKLFLWGQALYIIAKLLADELISPK | |
| | DIDPVQRYVPLKDQRNVSMRFSNQGPLENDLVVHVALIAESQRLQV | |
| | FLNTYGIQTQTPQQVEPIQIWPQQELVKAYLQLGINEKLGLSGRPDR | |
| | PIGCLGTSKIYRILGKTVVCYPIIFDLSDFYMSQDVFLLIDDIKNALQF | |
| | IKQYWKMHGRPLFLVLIREDNIRGSRFNPILDMLAALKKGIIGGVKV | |
| | HVDRLQTLISGAVVEQLDFLRISDTEELPEFKSFEELEPPKHSKVKRQ | |
| | SSTPSAPELGQQPDVNISEWKDKPTHEILQKLNDCSCLASQAILLGIL | |
| | LKREGPNFITKEGTVSDHIERVYRRAGSQKLWLAVRYGAAFTQKFS | |
| | SSIAPHITTFLVHGKQVTLGAFGHEEEVISNPLSPRVIQNIIYYKCNTH | |
| | DEREAVIQQELVIHIGWIISNNPELFSGMLKIRIGWIIHAMEYELQIRG | |
| | GDKPALDLYQLSPSEVKQLLLDILQPQQNGRCWLNRRQIDGSLNRT | |
| | PTGFYDRVWQILERTPNGIIVAGKHLPQQPTLSDMTMYEMNFSLLV | |
| | EDTLGNIDQPQYRQIVVELLMVVSIVLERNPELEFQDKVDLDRLVKE | |
| | AFNEFQKDQSRLKEIEKQDDMTSFYNTPPLGKRGTCSYLTKAVMNL | |
| | LLEGEVKPNNDDPCLIS | |
| 118 | MTLDVGPEDELPDWAAAKEFYQKYDPKDVIGRGVSSVVRRCVHRA | PHKG2 |
| | TGHEFAVKIMEVTAERLSPEQLEEVREATRRETHILRQVAGHPHIITL | |
| | IDSYESSSFMFLVFDLMRKGELFDYLTEKVALSEKETRSIMRSLLEA | |
| | VSFLHANNIVHRDLKPENILLDDNMQIRLSDFGFSCHLEPGEKLREL | |
| | CGTPGYLAPEILKCSMDETHPGYGKEVDLWACGVILFTLLAGSPPF | |
| | WHRRQILMLRMIMEGQYQFSSPEWDDRSSTVKDLISRLLQVDPEAR | |
| | LTAEQALQHPFFERCEGSQPWNLTPRQRFRVAVWTVLAAGRVALS | |
| | THRVRPLTKNALLRDPYALRSVRHLIDNCAFRLYGHWVKKGEQQN | |
| | RAALFQHRPPGPFPIMGPEEEGDSAAITEDEAVLVLG | |
| 119 | MAAQGYGYYRTVIFSAMFGGYSLYYFNRKTFSFVMPSLVEEIPLDK | SLC37A4 |
| | DDLGFITSSQSAAYAISKFVSGVLSDQMSARWLFSSGLLLVGLVNIF | |
| | FAWSSTVPVFAALWFLNGLAQGLGWPPCGKVLRKWFEPSQFGTW | |
| | WAILSTSMNLAGGLGPILATILAQSYSWRSTLALSGALCVVVSFLCL | |
| | LLIHNEPADVGLRNLDPMPSEGKKGSLKEESTLQELLLSPYLWVLST | |
| | GYLVVFGVKTCCTDWGQFFLIQEKGQSALVGSSYMSALEVGGLVG | |
| | SIAAGYLSDRAMAKAGLSNYGNPRHGLLLFMMAGMTVSMYLFRV | |
| | TVTSDSPKLWILVLGAVFGFSSYGPIALFGVIANESAPPNLCGTSHAI | |
| | VGLMANVGGFLAGLPFSTIAKHYSWSTAFWVAEVICAASTAAFFLL | |
| | RNIRTKMGRVSKKAE | |
| 120 | MAAPGPALCLFDVDGTLTAPRQKITKEMDDFLQKLRQKIKIGVVGG | PMM2 |
| | SDFEKVQEQLGNDVVEKYDYVFPENGLVAYKDGKLLCRQNIQSHL | |
| | GEALIQDLINYCLSYIAKIKLPKKRGTFIEFRNGMLNVSPIGRSCSQEE | |
| | RIEFYELDKKENIRQKFVADLRKEFAGKGLTFSIGGQISFDVFPDGW | |
| | DKRYCLRHVENDGYKTIYFFGDKTMPGGNDHEIFTDPRTMGYSVT | |
| | APEDTRRICELLFS | |
| 121 | MPSETPQAEVGPTGCPHRSGPHSAKGSLEKGSPEDKEAKEPLWIRPD | CBS |
| | APSRCTWQLGRPASESPHHHTAPAKSPKILPDILKKIGDTPMVRINKI | |
| | GKKFGLKCELLAKCEFFNAGGSVKDRISLRMIEDAERDGTLKPGDTI | |
| | IEPTSGNTGIGLALAAAVRGYRCIIVMPEKMSSEKVDVLRALGAEIV | |
| | RTPTNARFDSPESHVGVAWRLKNEIPNSHILDQYRNASNPLAHYDT | |
| | TADEILQQCDGKLDMLVASVGTGGTITGIARKLKEKCPGCRIIGVDP | |
| | EGSILAEPEELNQTEQTTYEVEGIGYDFIPTVLDRTVVDKWFKSNDE | |
| | EAFTFARMLIAQEGLLCGGSAGSTVAVAVKAAQELQEGQRCVVILP | |
| | DSVRNYMTKFLSDRWMLQKGFLKEEDLTEKKPWWWHLRVQELGL | |
| | SAPLTVLPTITCGHTIEILREKGFDQAPVVDEAGVILGMVTLGNMLS | |
| | SLLAGKVQPSDQVGKVIYKQFKQIRLTDTLGRLSHILEMDHFALVV | |
| | HEQIQYHSTGKSSQRQMVFGVVTAIDLLNFVAAQERDQK | |
| 122 | MSFIPVAEDSDFPIHNLPYGVFSTRGDPRPRIGVAIGDQILDLSIIKHLF | FAH |
| | TGPVLSKHQDVFNQPTLNSFMGLGQAAWKEARVFLQNLLSVSQAR | |
| | LRDDTELRKCAFISQASATMHLPATIGDYTDFYSSRQHATNVGIMFR | |
| | DKENALMPNWLHLPVGYHGRASSVVVSGTPIRRPMGQMKPDDSKP | |
| | PVYGACKLLDMELEMAFFVGPGNRLGEPIPISKAHEHIFGMVLMND | |
| | WSARDIQKWEYVPLGPFLGKSFGTTVSPWVVPMDALMPFAVPNPK | |
| | QDPRPLPYLCHDEPYTFDINLSVNLKGEGMSQAATICKSNFKYMYW | |
| | TMLQQLTHHSVNGCNLRPGDLLASGTISGPEPENFGSMLELSWKGT | |
| | KPIDLGNGQTRKFLLDGDEVIITGYCQGDGYRIGFGQCAGKVLPALL | |
| | PS | |
| 123 | MDPYMIQMSSKGNLPSILDVHVNVGGRSSVPGKMKGRKARWSVRP | TAT |
| | SDMAKKTFNPIRAIVDNMKVKPNPNKTMISLSIGDPTVFGNLPTDPE | |
| | VTQAMKDALDSGKYNGYAPSIGFLSSREEIASYYHCPEAPLEAKDVI | |
| | LTSGCSQAIDLCLAVLANPGQNILVPRPGFSLYKTLAESMGIEVKLY | |
| | NLLPEKSWEIDLKQLEYLIDEKTACLIVNNPSNPCGSVFSKRHLQKIL | |
| | AVAARQCVPILADEIYGDMVFSDCKYEPLATLSTDVPILSCGGLAKR | |
| | WLVPGWRLGWILIHDRRDIFGNEIRDGLVKLSQRILGPCTIVQGALK | |
| | SILCRTPGEFYHNTLSFLKSNADLCYGALAAIPGLRPVRPSGAMYLM | |
| | VGIEMEHFPEFENDVEFTERLVAEQSVHCLPATCFEYPNFIRVVITVP | |
| | EVMMLEACSRIQEFCEQHYHCAEGSQEECDK | |
| 124 | MSRSGTDPQQRQQASEADAAAATFRANDHQHIRYNPLQDEWVLVS | GALT |
| | AHRMKRPWQGQVEPQLLKTVPRHDPLNPLCPGAIRANGEVNPQYD | |
| | STFLFDNDFPALQPDAPSPGPSDHPLFQAKSARGVCKVMCFHPWSD | |
| | VTLPLMSVPEIRAVVDAWASVTEELGAQYPWVQIFENKGAMMGCS | |
| | NPHPHCQVWASSFLPDIAQREERSQQAYKSQHGEPLLMEYSRQELL | |
| | RKERLVLTSEHWLVLVPFWATWPYQTLLLPRRHVRRLPELTPAERD | |
| | DLASIMKKLLTKYDNLFETSFPYSMGWHGAPTGSEAGANWNHWQ | |
| | LHAHYYPPLLRSATVRKFMVGYEMLAQAQRDLTPEQAAERLRALP | |
| | EVHYHLGQKDRETATIA | |
| 125 | MAALRQPQVAELLAEARRAFREEFGAEPELAVSAPGRVNLIGEHTD | GALK1 |
| | YNQGLVLPMALELMTVLVGSPRKDGLVSLLTTSEGADEPQRLQFPL | |
| | PTAQRSLEPGTPRWANYVKGVIQYYPAAPLPGFSAVVVSSVPLGGG | |
| | LSSSASLEVATYTFLQQLCPDSGTIAARAQVCQQAEHSFAGMPCGI | |
| | MDQFISLMGQKGHALLIDCRSLETSLVPLSDPKLAVLITNSNVRHSL | |
| | ASSEYPVRRRQCEEVARALGKESLREVQLEELEAARDLVSKEGFRR | |
| | ARHVVGEIRRTAQAAAALRRGDYRAFGRLMVESHRSLRDDYEVSC | |
| | PELDQLVEAALAVPGVYGSRMTGGGFGGCTVTLLEASAAPHAMRH | |
| | IQEHYGGTATFYLSQAADGAKVLCL | |
| 126 | MAEKVLVTGGAGYIGSHTVLELLEAGYLPVVIDNFHNAFRGGGSLP | GALE |
| | ESLRRVQELTGRSVEFEEMDILDQGALQRLFKKYSFMAVIHFAGLK | |
| | AVGESVQKPLDYYRVNLTGTIQLLEIMKAHGVKNLVFSSSATVYGN | |
| | PQYLPLDEAHPTGGCTNPYGKSKFFIEEMIRDLCQADKTWNAVLLR | |
| | YFNPTGAHASGCIGEDPQGIPNNLMPYVSQVAIGRREALNVFGNDY | |
| | DTEDGTGVRDYIHVVDLAKGHIAALRKLKEQCGCRIYNLGTGTGYS | |
| | VLQMVQAMEKASGKKIPYKVVARREGDVAACYANPSLAQEELGW | |
| | TAALGLDRMCEDLWRWQKQNPSGFGTQA | |
| 127 | MAEQVALSRTQVCGILREELFQGDAFHQSDTHIFIIMGASGDLAKKK | G6PD |
| | IYPTIWWLFRDGLLPENTFIVGYARSRLTVADIRKQSEPFFKATPEEK | |
| | LKLEDFFARNSYVAGQYDDAASYQRLNSHMNALHLGSQANRLFYL | |
| | ALPPTVYEAVTKNIHESCMSQIGWNRIIVEKPFGRDLQSSDRLSNHIS | |
| | SLFREDQIYRIDHYLGKEMVQNLMVLRFANRIFGPIWNRDNIACVIL | |
| | TFKEPFGTEGRGGYFDEFGIIRDVMQNHLLQMLCLVAMEKPASTNS | |
| | DDVRDEKVKVLKCISEVQANNVVLGQYVGNPDGEGEATKGYLDD | |
| | PTVPRGSTTATFAAVVLYVENERWDGVPFILRCGKALNERKAEVRL | |
| | QFHDVAGDIFHQQCKRNELVIRVQPNEAVYTKMMTKKPGMFFNPE | |
| | ESELDLTYGNRYKNVKLPDAYERLILDVFCGSQMHFVRSDELREA | |
| | WRIFTPLLHQIELEKPKPIPYIYGSRGPTEADELMKRVGFQYEGTYK | |
| | WVNPHKL | |
| 128 | MAEDKSKRDSIEMSMKGCQTNNGFVHNEDILEQTPDPGSSTDNLKH | SLC3A1 |
| | STRGILGSQEPDFKGVQPYAGMPKEVLFQFSGQARYRIPREILFWLT | |
| | VASVLVLIAATIAIIALSPKCLDWWQEGPMYQIYPRSFKDSNKDGNG | |
| | DLKGIQDKLDYITALNIKTVWITSFYKSSLKDFRYGVEDFREVDPIFG | |
| | TMEDFENLVAAIHDKGLKLIIDFIPNHTSDKHIWFQLSRTRTGKYTD | |
| | YYIWHDCTHENGKTIPPNNWLSVYGNSSWHFDEVRNQCYFHQFMK | |
| | EQPDLNFRNPDVQEEIKEILRFWLTKGVDGFSLDAVKFLLEAKHLR | |
| | DEIQVNKTQIPDTVTQYSELYHDFTTTQVGMHDIVRSFRQTMDQYS | |
| | TEPGRYRFMGTEAYAESIDRTVMYYGLPFIQEADFPFNNYLSMLDT | |
| | VSGNSVYEVITSWMENMPEGKWPNWMIGGPDSSRLTSRLGNQYVN | |
| | VMNMLLFTLPGTPITYYGEEIGMGNIVAANLNESYDINTLRSKSPMQ | |
| | WDNSSNAGFSEASNTWLPTNSDYHTVNVDVQKTQPRSALKLYQDL | |
| | SLLHANELLLNRGWFCHLRNDSHYVVYTRELDGIDRIFIVVLNFGES | |
| | TLLNLHNMISGLPAKMRIRLSTNSADKGSKVDTSGIFLDKGEGLIFE | |
| | HNTKNLLHRQTAFRDRCFVSNRACYSSVLNILYTSC | |
| 129 | MGDTGLRKRREDEKSIQSQEPKTTSLQKELGLISGISIIVGTIIGSGIFV | SLC7A9 |
| | SPKSVLSNTEAVGPCLIIWAACGVLATLGALCFAELGTMITKSGGEY | |
| | PYLMEAYGPIPAYLFSWASLIVIKPTSFAIICLSFSEYVCAPFYVGCKP | |
| | PQIVVKCLAAAAILFISTVNSLSVRLGSYVQNIFTAAKLVIVAIIIISGL | |
| | VLLAQGNTKNFDNSFEGAQLSVGAISLAFYNGLWAYDGWNQLNYI | |
| | TEELRNPYRNLPLAIIIGIPLVTACYILMNVSYFTVMTATELLQSQAV | |
| | AVTFGDRVLYPASWIVPLFVAFSTIGAANGTCFTAGRLIYVAGREGH | |
| | MLKVLSYISVRRLTPAPAIIFYGIIATIYIIPGDINSLVNYFSFAAWLFY | |
| | GLTILGLIVMRFTRKELERPIKVPVVIPVLMTLISVFLVLAPIISKPTW | |
| | EYLYCVLFILSGLLFYFLFVHYKFGWAQKISKPITMHLQMLMEVVPP | |
| | EEDPE | |
| 130 | MVNEARGNSSLNPCLEGSASSGSESSKDSSRCSTPGLDPERHERLRE | MTHFR |
| | KMRRRLESGDKWFSLEFFPPRTAEGAVNLISRFDRMAAGGPLYIDV | |
| | TWHPAGDPGSDKETSSMMIASTAVNYCGLETILHMTCCRQRLEEIT | |
| | GHLHKAKQLGLKNIMALRGDPIGDQWEEEEGGFNYAVDLVKHIRS | |
| | EFGDYFDICVAGYPKGHPEAGSFEADLKHLKEKVSAGADFIITQLFF | |
| | EADTFFRFVKACTDMGITCPIVPGIFPIQGYHSLRQLVKLSKLEVPQE | |
| | IKDVIEPIKDNDAAIRNYGIELAVSLCQELLASGLVPGLHFYTLNREM | |
| | ATTEVLKRLGMWTEDPRRPLPWALSAHPKRREEDVRPIFWASRPKS | |
| | YIYRTQEWDEFPNGRWGNSSSPAFGELKDYYLFYLKSKSPKEELLK | |
| | MWGEELTSEESVFEVFVLYLSGEPNRNGHKVTCLPWNDEPLAAETS | |
| | LLKEELLRVNRQGILTINSQPNINGKPSSDPIVGWGPSGGYVFQKAY | |
| | LEFFTSRETAEALLQVLKKYELRVNYHLVNVKGENITNAPELQPNA | |
| | VTWGIFPGREIIQPTVVDPVSFMFWKDEAFALWIERWGKLYEEESPS | |
| | RTIIQYIHDNYFLVNLVDNDFPLDNCLWQVVEDTLELLNRPTQNAR | |
| | ETEAP | |
| 131 | MSPALQDLSQPEGLKKTLRDEINAILQKRIMVLDGGMGTMIQREKL | MTR |
| | NEEHFRGQEFKDHARPLKGNNDILSITQPDVIYQIHKEYLLAGADIIE | |
| | TNTFSSTSIAQADYGLEHLAYRMNMCSAGVARKAAEEVTLQTGIKR | |
| | FVAGALGPTNKTLSVSPSVERPDYRNITFDELVEAYQEQAKGLLDG | |
| | GVDILLIETIFDTANAKAALFALQNLFEEKYAPRPIFISGTIVDKSGRT | |
| | LSGQTGEGFVISVSHGEPLCIGLNCALGAAEMRPFIEIIGKCTTAYVL | |
| | CYPNAGLPNTFGDYDETPSMMAKHLKDFAMDGLVNIVGGCCGSTP | |
| | DHIREIAEAVKNCKPRVPPATAFEGHMLLSGLEPFRIGPYTNFVNIGE | |
| | RCNVAGSRKFAKLIMAGNYEEALCVAKVQVEMGAQVLDVNMDD | |
| | GMLDGPSAMTRFCNLIASEPDIAKVPLCIDSSNFAVIEAGLKCCQGK | |
| | CIVNSISLKEGEDDFLEKARKIKKYGAAMVVMAFDEEGQATETDTK | |
| | IRVCTRAYHLLVKKLGFNPNDIIFDPNILTIGTGMEEHNLYAINFIHAT | |
| | KVIKETLPGARISGGLSNLSFSFRGMEAIREAMHGVFLYHAIKSGMD | |
| | MGIVNAGNLPVYDDIHKELLQLCEDLIWNKDPEATEKLLRYAQTQG | |
| | TGGKKVIQTDEWRNGPVEERLEYALVKGIEKHIIEDTEEARLNQKK | |
| | YPRPLNIIEGPLMNGMKIVGDLFGAGKMFLPQVIKSARVMKKAVGH | |
| | LIPFMEKEREETRVLNGTVEEEDPYQGTIVLATVKGDVHDIGKNIVG | |
| | VVLGCNNFRVIDLGVMTPCDKILKAALDHKADIIGLSGLITPSLDEMI | |
| | FVAKEMERLAIRIPLLIGGATTSKTHTAVKIAPRYSAPVIHVLDASKS | |
| | VVVCSQLLDENLKDEYFEEIMEEYEDIRQDHYESLKERRYLPLSQAR | |
| | KSGFQMDWLSEPHPVKPTFIGTQVFEDYDLQKLVDYIDWKPFFDV | |
| | WQLRGKYPNRGFPKIFNDKTVGGEARKVYDDAHNMLNTLISQKKL | |
| | RARGVVGFWPAQSIQDDIHLYAEAAVPQAAEPIATFYGLRQQAEKD | |
| | SASTEPYYCLSDFIAPLHSGIRDYLGLFAVACFGVEELSKAYEDDGD | |
| | DYSSIMVKALGDRLAEAFAEELHERVRRELWAYCGSEQLDVADLR | |
| | RLRYKGIRPAPGYPSQPDHTEKLTMWRLADIEQSTGIRLTESLAMAP | |
| | ASAVSGLYFSNLKSKYFAVGKISKDQVEDYALRKNISVAEVEKWLG | |
| | PILGYDTD | |
| 132 | MGAASVRAGARLVEVALCSFTVTCLEVMRRFLLLYATQQGQAKAI | MTRR |
| | AEEICEQAVVHGFSADLHCISESDKYDLKTETAPLVVVVSTTGTGDP | |
| | PDTARKFVKEIQNQTLPVDFFAHLRYGLLGLGDSEYTYFCNGGKIID | |
| | KRLQELGARHFYDTGHADDCVGLELVVEPWIAGLWPALRKHFRSS | |
| | RGQEEISGALPVASPASSRTDLVKSELLHIESQVELLRFDDSGRKDSE | |
| | VLKQNAVNSNQSNVVIEDFESSLTRSVPPLSQASLNIPGLPPEYLQVH | |
| | LQESLGQEESQVSVTSADPVFQVPISKAVQLTTNDAIKTTLLVELDIS | |
| | NTDFSYQPGDAFSVICPNSDSEVQSLLQRLQLEDKREHCVLLKIKAD | |
| | TKKKGATLPQHIPAGCSLQFIFTWCLEIRAIPKKAFLRALVDYTSDSA | |
| | EKRRLQELCSKQGAADYSRFVRDACACLLDLLLAFPSCQPPLSLLLE | |
| | HLPKLQPRPYSCASSSLFHPGKLHFVFNIVEFLSTATTEVLRKGVCTG | |
| | WLALLVASVLQPNIHASHEDSGKALAPKISISPRTTNSFHLPDDPSIPI | |
| | IMVGPGTGIAPFIGFLQHREKLQEQHPDGNFGAMWLFFGCRHKDRD | |
| | YLFRKELRHFLKHGILTHLKVSFSRDAPVGEEEAPAKYVQDNIQLH | |
| | GQQVARILLQENGHIYVCGDAKNMAKDVHDALVQIISKEVGVEKL | |
| | EAMKTLATLKEEKRYLQDIWS | |
| 133 | MPEQERQITAREGASRKILSKLSLPTRAWEPAMKKSFAFDNVGYEG | ATP7B |
| | GLDGLGPSSQVATSTVRILGMTCQSCVKSIEDRISNLKGIISMKVSLE | |
| | QGSATVKYVPSVVCLQQVCHQIGDMGFEASIAEGKAASWPSRSLPA | |
| | QEAVVKLRVEGMTCQSCVSSIEGKVRKLQGVVRVKVSLSNQEAVIT | |
| | YQPYLIQPEDLRDHVNDMGFEAAIKSKVAPLSLGPIDIERLQSTNPK | |
| | RPLSSANQNFNNSETLGHQGSHVVTLQLRIDGMHCKSCVLNIEENIG | |
| | QLLGVQSIQVSLENKTAQVKYDPSCTSPVALQRAIEALPPGNFKVSL | |
| | PDGAEGSGTDHRSSSSHSPGSPPRNQVQGTCSTTLIAIAGMTCASCV | |
| | HSIEGMISQLEGVQQISVSLAEGTATVLYNPSVISPEELRAAIEDMGF | |
| | EASVVSESCSTNPLGNHSAGNSMVQTTDGTPTSVQEVAPHTGRLPA | |
| | NHAPDILAKSPQSTRAVAPQKCFLQIKGMTCASCVSNIERNLQKEAG | |
| | VLSVLVALMAGKAEIKYDPEVIQPLEIAQFIQDLGFEAAVMEDYAG | |
| | SDGNIELTITGMTCASCVHNIESKLTRTNGITYASVALATSKALVKF | |
| | DPEIIGPRDIIKIIEEIGFHASLAQRNPNAHHLDHKMEIKQWKKSFLCS | |
| | LVFGIPVMALMIYMLIPSNEPHQSMVLDHNIIPGLSILNLIFFILCTFV | |
| | QLLGGWYFYVQAYKSLRHRSANMDVLIVLATSIAYVYSLVILVVA | |
| | VAEKAERSPVTFFDTPPMLFVFIALGRWLEHLAKSKTSEALAKLMS | |
| | LQATEATVVTLGEDNLIIREEQVPMELVQRGDIVKVVPGGKFPVDG | |
| | KVLEGNTMADESLITGEAMPVTKKPGSTVIAGSINAHGSVLIKATHV | |
| | GNDTTLAQIVKLVEEAQMSKAPIQQLADRFSGYFVPFIIIMSTLTLVV | |
| | WIVIGFIDFGVVQRYFPNPNKHISQTEVIIRFAFQTSITVLCIACPCSLG | |
| | LATPTAVMVGTGVAAQNGILIKGGKPLEMAHKIKTVMFDKTGTITH | |
| | GVPRVMRVLLLGDVATLPLRKVLAVVGTAEASSEHPLGVAVTKYC | |
| | KEELGTETLGYCTDFQAVPGCGIGCKVSNVEGILAHSERPLSAPASH | |
| | LNEAGSLPAEKDAVPQTFSVLIGNREWLRRNGLTISSDVSDAMTDH | |
| | EMKGQTAILVAIDGVLCGMIAIADAVKQEAALAVHTLQSMGVDVV | |
| | LITGDNRKTARAIATQVGINKVFAEVLPSHKVAKVQELQNKGKKVA | |
| | MVGDGVNDSPALAQADMGVAIGTGTDVAIEAADVVLIRNDLLDVV | |
| | ASIHLSKRTVRRIRINLVLALIYNLVGIPIAAGVFMPIGIVLQPWMGS | |
| | AAMAASSVSVVLSSLQLKCYKKPDLERYEAQAHGHMKPLTASQVS | |
| | VHIGMDDRWRDSPRATPWDQVSYVSQVSLSSLTSDKPSRHSAAAD | |
| | DDGDKWSLLLNGRDEEQYI | |
| 134 | MATRSPGVVISDDEPGYDLDLFCIPNHYAEDLERVFIPHGLIMDRTE | HPRT1 |
| | RLARDVMKEMGGHHIVALCVLKGGYKFFADLLDYIKALNRNSDRS | |
| | IPMTVDFIRLKSYCNDQSTGDIKVIGGDDLSTLTGKNVLIVEDIIDTG | |
| | KTMQTLLSLVRQYNPKMVKVASLLVKRTPRSVGYKPDFVGFEIPDK | |
| | FVVGYALDYNEYFRDLNHVCVISETGKAKYKA | |
| 135 | MGEPGQSPSPRSSHGSPPTLSTLTLLLLLCGHAHSQCKILRCNAEYVS | HJV |
| | STLSLRGGGSSGALRGGGGGGRGGGVGSGGLCRALRSYALCTRRT | |
| | ARTCRGDLAFHSAVHGIEDLMIQHNCSRQGPTAPPPPRGPALPGAGS | |
| | GLPAPDPCDYEGRFSRLHGRPPGFLHCASFGDPHVRSFHHHFHTCR | |
| | VQGAWPLLDNDFLFVQATSSPMALGANATATRKLTIIFKNMQECID | |
| | QKVYQAEVDNLPVAFEDGSINGGDRPGGSSLSIQTANPGNHVEIQA | |
| | AYIGTTIIIRQTAGQLSFSIKVAEDVAMAFSAEQDLQLCVGGCPPSQR | |
| | LSRSERNRRGAITIDTARRLCKEGLPVEDAYFHSCVFDVLISGDPNFT | |
| | VAAQAALEDARAFLPDLEKLHLFPSDAGVPLSSATLLAPLLSGLFVL | |
| | WLCIQ | |
| 136 | MALSSQIWAACLLLLLLLASLTSGSVFPQQTGQLAELQPQDRAGAR | HAMP |
| | ASWMPMFQRRRRRDTHFPICIFCCGCCHRSKCGMCCKT | |
| 137 | MRSPRTRGRSGRPLSLLLALLCALRAKVCGASGQFELEILSMQNVN | JAG1 |
| | GELQNGNCCGGARNPGDRKCTRDECDTYFKVCLKEYQSRVTAGGP | |
| | CSFGSGSTPVIGGNTFNLKASRGNDRNRIVLPFSFAWPRSYTLLVEA | |
| | WDSSNDTVQPDSIIEKASHSGMINPSRQWQTLKQNTGVAHFEYQIR | |
| | VTCDDYYYGFGCNKFCRPRDDFFGHYACDQNGNKTCMEGWMGPE | |
| | CNRAICRQGCSPKHGSCKLPGDCRCQYGWQGLYCDKCIPHPGCVH | |
| | GICNEPWQCLCETNWGGQLCDKDLNYCGTHQPCLNGGTCSNTGPD | |
| | KYQCSCPEGYSGPNCEIAEHACLSDPCHNRGSCKETSLGFECECSPG | |
| | WTGPTCSTNIDDCSPNNCSHGGTCQDLVNGFKCVCPPQWTGKTCQ | |
| | LDANECEAKPCVNAKSCKNLIASYYCDCLPGWMGQNCDININDCL | |
| | GQCQNDASCRDLVNGYRCICPPGYAGDHCERDIDECASNPCLNGG | |
| | HCQNEINRFQCLCPTGFSGNLCQLDIDYCEPNPCQNGAQCYNRASD | |
| | YFCKCPEDYEGKNCSHLKDHCRTTPCEVIDSCTVAMASNDTPEGVR | |
| | YISSNVCGPHGKCKSQSGGKFTCDCNKGFTGTYCHENINDCESNPC | |
| | RNGGTCIDGVNSYKCICSDGWEGAYCETNINDCSQNPCHNGGTCRD | |
| | LVNDFYCDCKNGWKGKTCHSRDSQCDEATCNNGGTCYDEGDAFK | |
| | CMCPGGWEGTTCNIARNSSCLPNPCHNGGTCVVNGESFTCVCKEG | |
| | WEGPICAQNTNDCSPHPCYNSGTCVDGDNWYRCECAPGFAGPDCR | |
| | ININECQSSPCAFGATCVDEINGYRCVCPPGHSGAKCQEVSGRPCIT | |
| | MGSVIPDGAKWDDDCNTCQCLNGRIACSKVWCGPRPCLLHKGHSE | |
| | CPSGQSCIPILDDQCFVHPCTGVGECRSSSLQPVKTKCTSDSYYQDN | |
| | CANITFTFNKEMMSPGLTTEHICSELRNLNILKNVSAEYSIYIACEPSP | |
| | SANNEIHVAISAEDIRDDGNPIKEITDKIIDLVSKRDGNSSLIAAVAEV | |
| | RVQRRPLKNRTDFLVPLLSSVLTVAWICCLVTAFYWCLRKRRKPGS | |
| | HTHSASEDNTTNNVREQLNQIKNPIEKHGANTVPIKDYENKNSKMS | |
| | KIRTHNSEVEEDDMDKHQQKARFAKQPAYTLVDREEKPPNGTPTK | |
| | HPNWTNKQDNRDLESAQSLNRMEYIV | |
| 138 | MASHRLLLLCLAGLVFVSEAGPTGTGESKCPLMVKVLDAVRGSPAI | TTR |
| | NVAVHVFRKAADDTWEPFASGKTSESGELHGLTTEEEFVEGIYKVE | |
| | IDTKSYWKALGISPFHEHAEVVFTANDSGPRRYTIAALLSPYSYSTT | |
| | AVVTNPKE | |
| 139 | MASHKLLVTPPKALLKPLSIPNQLLLGPGPSNLPPRIMAAGGLQMIG | AGXT |
| | SMSKDMYQIMDEIKEGIQYVFQTRNPLTLVISGSGHCALEAALVNV | |
| | LEPGDSFLVGANGIWGQRAVDIGERIGARVHPMTKDPGGHYTLQEV | |
| | EEGLAQHKPVLLFLTHGESSTGVLQPLDGFGELCHRYKCLLLVDSV | |
| | ASLGGTPLYMDRQGIDILYSGSQKALNAPPGTSLISFSDKAKKKMYS | |
| | RKTKPFSFYLDIKWLANFWGCDDQPRMYHHTIPVISLYSLRESLALI | |
| | AEQGLENSWRQHREAAAYLHGRLQALGLQLFVKDPALRLPTVTTV | |
| | AVPAGYDWRDIVSYVIDHFDIEIMGGLGPSTGKVLRIGLLGCNATRE | |
| | NVDRVTEALRAALQHCPKKKL | |
| 140 | MKMRFLGLVVCLVLWTLHSEGSGGKLTAVDPETNMNVSEIISYWG | LIPA |
| | FPSEEYLVETEDGYILCLNRIPHGRKNHSDKGPKPVVFLQHGLLADS | |
| | SNWVTNLANSSLGFILADAGFDVWMGNSRGNTWSRKHKTLSVSQD | |
| | EFWAFSYDEMAKYDLPASINFILNKTGQEQVYYVGHSQGTTIGFIAF | |
| | SQIPELAKRIKMFFALGPVASVAFCTSPMAKLGRLPDHLIKDLFGDK | |
| | EFLPQSAFLKWLGTHVCTHVILKELCGNLCFLLCGFNERNLNMSRV | |
| | DVYTTHSPAGTSVQNMLHWSQAVKFQKFQAFDWGSSAKNYFHYN | |
| | QSYPPTYNVKDMLVPTAVWSGGHDWLADVYDVNILLTQITNLVFH | |
| | ESIPEWEHLDFIWGLDAPWRLYNKIINLMRKYQ | |
| 141 | MASRLTLLTLLLLLLAGDRASSNPNATSSSSQDPESLQDRGEGKVAT | SERPING1 |
| | TVISKMLFVEPILEVSSLPTTNSTTNSATKITANTTDEPTTQPTTEPTT | |
| | QPTIQPTQPTTQLPTDSPTQPTTGSFCPGPVTLCSDLESHSTEAVLGD | |
| | ALVDFSLKLYHAFSAMKKVETNMAFSPFSIASLLTQVLLGAGENTK | |
| | TNLESILSYPKDFTCVHQALKGFTTKGVTSVSQIFHSPDLAIRDTFVN | |
| | ASRTLYSSSPRVLSNNSDANLELINTWVAKNTNNKISRLLDSLPSDT | |
| | RLVLLNAIYLSAKWKTTFDPKKTRMEPFHFKNSVIKVPMMNSKKYP | |
| | VAHFIDQTLKAKVGQLQLSHNLSLVILVPQNLKHRLEDMEQALSPS | |
| | VFKAIMEKLEMSKFQPTLLTLPRIKVTTSQDMLSIMEKLEFFDFSYD | |
| | LNLCGLTEDPDLQVSAMQHQTVLELTETGVEAAAASAISVARTLLV | |
| | FEVQQPFLFVLWDQQHKFPVFMGRVYDPRA | |
| 142 | MGSPLRFDGRVVLVTGAGAGLGRAYALAFAERGALVVVNDLGGD | HSD17B4 |
| | FKGVGKGSLAADKVVEEIRRRGGKAVANYDSVEEGEKVVKTALDA | |
| | FGRIDVVVNNAGILRDRSFARISDEDWDIIHRVHLRGSFQVTRAAWE | |
| | HMKKQKYGRIIMTSSASGIYGNFGQANYSAAKLGLLGLANSLAIEG | |
| | RKSNIHCNTIAPNAGSRMTQTVMPEDLVEALKPEYVAPLVLWLCHE | |
| | SCEENGGLFEVGAGWIGKLRWERTLGAIVRQKNHPMTPEAVKANW | |
| | KKICDFENASKPQSIQESTGSIIEVLSKIDSEGGVSANHTSRATSTATS | |
| | GFAGAIGQKLPPFSYAYTELEAIMYALGVGASIKDPKDLKFIYEGSS | |
| | DFSCLPTFGVIIGQKSMMGGGLAEIPGLSINFAKVLHGEQYLELYKP | |
| | LPRAGKLKCEAVVADVLDKGSGVVIIMDVYSYSEKELICHNQFSLF | |
| | LVGSGGFGGKRTSDKVKVAVAIPNRPPDAVLTDTTSLNQAALYRLS | |
| | GDWNPLHIDPNFASLAGFDKPILHGLCTFGFSARRVLQQFADNDVS | |
| | RFKAIKARFAKPVYPGQTLQTEMWKEGNRIHFQTKVQETGDIVISN | |
| | AYVDLAPTSGTSAKTPSEGGKLQSTFVFEEIGRRLKDIGPEVVKKVN | |
| | AVFEWHITKGGNIGAKWTIDLKSGSGKVYQGPAKGAADTTIILSDE | |
| | DFMEVVLGKLDPQKAFFSGRLKARGNIMLSQKLQMILKDYAKL | |
| 143 | MEANGLGPQGFPELKNDTFLRAAWGEETDYTPVWCMRQAGRYLP | UROD |
| | EFRETRAAQDFFSTCRSPEACCELTLQPLRRFPLDAAIIFSDILVVPQA | |
| | LGMEVTMVPGKGPSFPEPLREEQDLERLRDPEVVASELGYVFQAITL | |
| | TRQRLAGRVPLIGFAGAPWTLMTYMVEGGGSSTMAQAKRWLYQR | |
| | PQASHQLLRILTDALVPYLVGQVVAGAQALQLFESHAGHLGPQLFN | |
| | KFALPYIRDVAKQVKARLREAGLAPVPMIIFAKDGHFALEELAQAG | |
| | YEVVGLDWTVAPKKARECVGKTVTLQGNLDPCALYASEEEIGQLV | |
| | KQMLDDFGPHRYIANLGHGLYPDMDPEHVGAFVDAVHKHSRLLR | |
| | QN | |
| 144 | MGPRARPALLLLMLLQTAVLQGRLLRSHSLHYLFMGASEQDLGLSL | HFE |
| | FEALGYVDDQLFVFYDHESRRVEPRTPWVSSRISSQMWLQLSQSLK | |
| | GWDHMFTVDFWTIMENHNHSKESHTLQVILGCEMQEDNSTEGYW | |
| | KYGYDGQDHLEFCPDTLDWRAAEPRAWPTKLEWERHKIRARQNR | |
| | AYLERDCPAQLQQLLELGRGVLDQQVPPLVKVTHHVTSSVTTLRCR | |
| | ALNYYPQNITMKWLKDKQPMDAKEFEPKDVLPNGDGTYQGWITL | |
| | AVPPGEEQRYTCQVEHPGLDQPLIVIWEPSPSGTLVIGVISGIAVFVVI | |
| | LFIGILFIILRKRQGSRGAMGHYVLAERE | |
| 145 | MESKALLVLTLAVWLQSLTASRGGVAAADQRRDFIDIESKFALRTP | LPL |
| | EDTAEDTCHLIPGVAESVATCHFNHSSKTFMVIHGWTVTGMYESW | |
| | VPKLVAALYKREPDSNVIVVDWLSRAQEHYPVSAGYTKLVGQDVA | |
| | RFINWMEEEFNYPLDNVHLLGYSLGAHAAGIAGSLTNKKVNRITGL | |
| | DPAGPNFEYAEAPSRLSPDDADFVDVLHTFTRGSPGRSIGIQKPVGH | |
| | VDIYPNGGTFQPGCNIGEAIRVIAERGLGDVDQLVKCSHERSIHLFID | |
| | SLLNEENPSKAYRCSSKEAFEKGLCLSCRKNRCNNLGYEINKVRAK | |
| | RSSKMYLKTRSQMPYKVFHYQVKIHFSGTESETHTNQAFEISLYGT | |
| | VAESENIPFTLPEVSTNKTYSFLIYTEVDIGELLMLKLKWKSDSYFS | |
| | WSDWWSSPGFAIQKIRVKAGETQKKVIFCSREKVSHLQKGKAPAVF | |
| | VKCHDKSLNKKSG | |
| 146 | MRPVRLMKVFVTRRIPAEGRVALARAADCEVEQWDSDEPIPAKELE | GRHPR |
| | RGVAGAHGLLCLLSDHVDKRILDAAGANLKVISTMSVGIDHLALDE | |
| | IKKRGIRVGYTPDVLTDTTAELAVSLLLTTCRRLPEAIEEVKNGGWT | |
| | SWKPLWLCGYGLTQSTVGIIGLGRIGQAIARRLKPFGVQRFLYTGRQ | |
| | PRPEEAAEFQAEFVSTPELAAQSDFIVVACSLTPATEGLCNKDFFQK | |
| | MKETAVFINISRGDVVNQDDLYQALASGKIAAAGLDVTSPEPLPTN | |
| | HPLLTLKNCVILPHIGSATHRTRNTMSLLAANNLLAGLRGEPMPSEL | |
| | KL | |
| 147 | MLGPQVWSSVRQGLSRSLSRNVGVWASGEGKKVDIAGIYPPVTTPF | HOGA1 |
| | TATAEVDYGKLEENLHKLGTFPFRGFVVQGSNGEFPFLTSSERLEVV | |
| | SRVRQAMPKNRLLLAGSGCESTQATVEMTVSMAQVGADAAMVVT | |
| | PCYYRGRMSSAALIHHYTKVADLSPIPVVLYSVPANTGLDLPVDAV | |
| | VTLSQHPNIVGMKDSGGDVTRIGLIVHKTRKQDFQVLAGSAGFLMA | |
| | SYALGAVGGVCALANVLGAQVCQLERLCCTGQWEDAQKLQHRLIE | |
| | PNAAVTRRFGIPGLKKIMDWFGYYGGPCRAPLQELSPAEEEALRMD | |
| | FTSNGWL | |
| 148 | MGPWGWKLRWTVALLLAAAGTAVGDRCERNEFQCQDGKCISYK | LDLR |
| | WVCDGSAECQDGSDESQETCLSVTCKSGDFSCGGRVNRCIPQFWRC | |
| | DGQVDCDNGSDEQGCPPKTCSQDEFRCHDGKCISRQFVCDSDRDCL | |
| | DGSDEASCPVLTCGPASFQCNSSTCIPQLWACDNDPDCEDGSDEWP | |
| | QRCRGLYVFQGDSSPCSAFEFHCLSGECIHSSWRCDGGPDCKDKSD | |
| | EENCAVATCRPDEFQCSDGNCIHGSRQCDREYDCKDMSDEVGCVN | |
| | VTLCEGPNKFKCHSGECITLDKVCNMARDCRDWSDEPIKECGTNEC | |
| | LDNNGGCSHVCNDLKIGYECLCPDGFQLVAQRRCEDIDECQDPDTC | |
| | SQLCVNLEGGYKCQCEEGFQLDPHTKACKAVGSIAYLFFTNRHEVR | |
| | KMTLDRSEYTSLIPNLRNVVALDTEVASNRIYWSDLSQRMICSTQLD | |
| | RAHGVSSYDTVISRDIQAPDGLAVDWIHSNIYWTDSVLGTVSVADT | |
| | KGVKRKTLFRENGSKPRAIVVDPVHGFMYWTDWGTPAKIKKGGLN | |
| | GVDIYSLVTENIQWPNGITLDLLSGRLYWVDSKLHSISSIDVNGGNR | |
| | KTILEDEKRLAHPFSLAVFEDKVFWTDIINEAIFSANRLTGSDVNLLA | |
| | ENLLSPEDMVLFHNLTQPRGVNWCERTTLSNGGCQYLCLPAPQINP | |
| | HSPKFTCACPDGMLLARDMRSCLTEAEAAVATQETSTVRLKVSSTA | |
| | VRTQHTTTRPVPDTSRLPGATPGLTTVEIVTMSHQALGDVAGRGNE | |
| | KKPSSVRALSIVLPIVLLVFLCLGVFLLWKNWRLKNINSINFDNPVY | |
| | QKTTEDEVHICHNQDGYSYPSRQMVSLEDDVA | |
| 149 | MLWSGCRRFGARLGCLPGGLRVLVQTGHRS | ACAD8 |
| | LTSCIDPSMGLNEEQKEFQKVAFDFAAREM | |
| | APNMAEWDQKELFPVDVMRKAAQLGFGGVY | |
| | IQTDVGGSGLSRLDTSVIFEALATGCTSTT | |
| | AYISIHNMCAWMIDSFGNEE | |
| | QRHKFCPPLCTMEKFASYCLTEPGSGSDAA | |
| | SLLTSAKKQGDHYILNGSKAFISGAGESDI | |
| | YVVMCRTGGPGPKGISCIVVEKGTPGLSFG | |
| | KKEKKVGWNSQPTRAVIFEDCAVPVANRIG | |
| | SEGQGFLIAVRGLNGGRINIASCSLGAAHA | |
| | SVILTRDHLNVRKQFGEPLASNQYLQFTLADMATRLVAARLMVRN | |
| | AAVALQEERKDAVALCSMAKLFATDECFAICNQALQMHGGYGYL | |
| | KDYAVQQYVRDSRVHQILEGSNEVMRILISRSLLQE | |
| 150 | MEGLAVRLLRGSRLLRRNFLTCLSSWKIPPHVSKSSQSEALLNITNN | ACADSB |
| | GIHFAPLQTFTDEEMMIKSSVKKFAQEQIAPLVSTMDENSKMEKSVI | |
| | QGLFQQGLMGIEVDPEYGGTGASFLSTVLVIEELAKVDASVAVFCEI | |
| | QNTLINTLIRKHGTEEQKATYLPQLTTEKVGSFCLSEAGAGSDSFAL | |
| | KTRADKEGDYYVLNGSKMWISSAEHAGLFLVMANVDPTIGYKGIT | |
| | SFLVDRDTPGLHIGKPENKLGLRASSTCPLTFENVKVPEANILGQIGH | |
| | GYKYAIGSLNEGRIGIAAQMLGLAQGCFDYTIPYIKERIQFGKRLFDF | |
| | QGLQHQVAHVATQLEAARLLTYNAARLLEAGKPFIKEASMAKYYA | |
| | SEIAGQTTSKCIEWMGGVGYTKDYPVEKYFRDAKIGTIYEGASNIQL | |
| | NTIAKHIDAEY | |
| 151 | MAVLAALLRSGARSRSPLLRRLVQEIRYVERSYVSKPTLKEVVIVSA | ACAT1 |
| | TRTPIGSFLGSLSLLPATKLGSIAIQGAIEKAGIPKEEVKEAYMGNVL | |
| | QGGEGQAPTRQAVLGAGLPISTPCTTINKVCASGMKAIMMASQSLM | |
| | CGHQDVMVAGGMESMSNVPYVMNRGSTPYGGVKLEDLIVKDGLT | |
| | DVYNKIHMGSCAENTAKKLNIARNEQDAYAINSYTRSKAAWEAGK | |
| | FGNEVIPVTVTVKGQPDVVVKEDEEYKRVDFSKVPKLKTVFQKEN | |
| | GTVTAANASTLNDGAAALVLMTADAAKRLNVTPLARIVAFADAAV | |
| | EPIDFPIAPVYAASMVLKDVGLKKEDIAMWEVNEAFSLVVLANIKM | |
| | LEIDPQKVNINGGAVSLGHPIGMSGARIVGHLTHALKQGEYGLASIC | |
| | NGGGGASAMLIQKL | |
| 152 | MLPHVVLTFRRLGCALASCRLAPARHRGSGLLHTAPVARSDRSAPV | ACSF3 |
| | FTRALAFGDRIALDQHGRHTYRELYSRSLRLSQEICRLCGCVGGDLR | |
| | EERVSFLCANDASYVVAQWASWMSGGVAVPLYRKHPAAQLEYVI | |
| | CDSQSSVVLASQEYLELLSPVVRKLGVPLLPLTPAIYTGAVEEPAEV | |
| | PVPEQGWRNKGAMIIYTSGTTGRPKGVLSTHQNIRAVVTGLVHKW | |
| | AWTKDDVILHVLPLHHVHGVVNALLCPLWVGATCVMMPEFSPQQ | |
| | VWEKFLSSETPRINVFMAVPTIYTKLMEYYDRHFTQPHAQDFLRAV | |
| | CEEKIRLMVSGSAALPLPVLEKWKNITGHTLLERYGMTEIGMALSG | |
| | PLTTAVRLPGSVGTPLPGVQVRIVSENPQREACSYTIHAEGDERGTK | |
| | VTPGFEEKEGELLVRGPSVFREYWNKPEETKSAFTLDGWFKTGDTV | |
| | VFKDGQYWIRGRTSVDIIKTGGYKVSALEVEWHLLAHPSITDVAVIG | |
| | VPDMTWGQRVTAVVTLREGHSLSHRELKEWARNVLAPYAVPSELV | |
| | LVEEIPRNQMGKIDKKALIRHFHPS | |
| 153 | MTSCHIAEEHIQKVAIFGGTHGNELTGVFLVKHWLENGAEIQRTGLE | ASPA |
| | VKPFITNPRAVKKCTRYIDCDLNRIFDLENLGKKMSEDLPYEVRRAQ | |
| | EINHLFGPKDSEDSYDIIFDLHNTTSNMGCTLILEDSRNNFLIQMFHYI | |
| | KTSLAPLPCYVYLIEHPSLKYATTRSIAKYPVGIEVGPQPQGVLRADI | |
| | LDQMRKMIKHALDFIHHFNEGKEFPPCAIEVYKIIEKVDYPRDENGE | |
| | IA | |
| | AIIHPNLQDQDWKPLHPGDPMFLTLDGKTIPLGGDCTVYPVFVNEA | |
| | AYYEKKEAFAKTTKLTLNAKSIRCCLH | |
| 154 | MAAAVAAAPGALGSLHAGGARLVAACSAWLCPGLRLPGSLAGRR | AUH |
| | AGPAIWAQGWVPAAGGPAPKRGYSSEMKTEDELRVRHLEEENRGI | |
| | VVLGINRAYGKNSLSKNLIKMLSKAVDALKSDKKVRTIIIRSEVPGIF | |
| | CAGADLKERAKMSSSEVGPFVSKIRAVINDIANLPVPTIAAIDGLAL | |
| | GGGLELALACDIRVAASSAKMGLVETKLAIIPGGGGTQRLPRAIGMS | |
| | LAKELIFSARVLDGKEAKAVGLISHVLEQNQEGDAAYRKALDLARE | |
| | FLPQGPVAMRVAKLAINQGMEVDLVTGLAIEEACYAQTIPTKDRLE | |
| | GLLAFKEKRPPRYKGE | |
| 155 | MASTVVAVGLTIAAAGFAGRYVLQAMKHMEPQVKQVFQSLPKSAF | DNAJC19 |
| | SGGYYRGGFEPKMTKREAALILGVSPTANKGKIRDAHRRIMLLNHP | |
| | DKGGSPYIAAKINEAKDLLEGQAKK | |
| 156 | MAEAVLRVARRQLSQRGGSGAPILLRQMFEPVSCTFTYLLGDRESR | ETHE1 |
| | EAVLIDPVLETAPRDAQLIKELGLRLLYAVNTHCHADHITGSGLLRS | |
| | LLPGCQSVISRLSGAQADLHIEDGDSIRFGRFALETRASPGHTPGCVT | |
| | FVLNDHSMAFTGDALLIRGCGRTDFQQGCAKTLYHSVHEKIFTLPG | |
| | DCLIYPAHDYHGFTVSTVEEERTLNPRLTLSCEEFVKIMGNLNLPKP | |
| | QQIDFAVPANMRCGVQTPTA | |
| 157 | MADQAPFDTDVNTLTRFVMEEGRKARGTGELTQLLNSLCTAVKAIS | FBP1 |
| | SAVRKAGIAHLYGIAGSTNVTGDQVKKLDVLSNDLVMNMLKSSFA | |
| | TCVLVSEEDKHAIIVEPEKRGKYVVCFDPLDGSSNIDCLVSVGTIFGI | |
| | YRKKSTDEPSEKDALQPGRNLVAAGYALYGSATMLVLAMDCGVN | |
| | CFMLDPAIGEFILVDKDVKIKKKGKIYSLNEGYARDFDPAVTEYIQR | |
| | KKFPPDNSAPYGARYVGSMVADVHRTLVYGGIFLYPANKKSPNGK | |
| | LRLLYECNPMAYVMEKAGGMATTGKEAVLDVIPTDIHQRAPVILGS | |
| | PDDVLEFLKVYEKHSAQ | |
| 158 | MSQLVECVPNFSEGKNQEVIDAISGAITQTPGCVLLDVDAGPSTNRT | FTCD |
| | VYTFVGPPECVVEGALNAARVASRLIDMSRHQGEHPRMGALDVCP | |
| | FIPVRGVSVDECVLCAQAFGQRLAEELDVPVYLYGEAARMDSRRTL | |
| | PAIRAGEYEALPKKLQQADWAPDFGPSSFVPSWGATATGARKFLIA | |
| | FNINLLGTKEQAHRIALNLREQGRGKDQPGRLKKVQGIGWYLDEKN | |
| | LAQVSTNLLDFEVTALHTVYEETCREAQELSLPVVGSQLVGLVPLK | |
| | ALLDAAAFYCEKENLFILEEEQRI | |
| | RLVVSRLGLDSLCPFSPKERIIEYLVPERGPERGLGSKSLRAFVGEVG | |
| | ARSAAPGGGSVAAAAAAMGAALGSMVGLMTYGRRQFQSLDTTMR | |
| | RLIPPFREASAKLTTLVDADAEAFTAYLEAMRLPKNTPEEKDRRTA | |
| | ALQEGLRRAVSVPLTLAETVASLWPALQELARCGNLACRSDLQVA | |
| | AKALEMGVFGAYFNVLINLRDITDEAFKDQIHHRVSSLLQEAKTQA | |
| | ALVLDCLETRQE | |
| 159 | MATNWGSLLQDKQQLEELARQAVDRALAEGVLLRTSQEPTSSEVV | GSS |
| | SYAPFTLFPSLVPSALLEQAYAVQMDFNLLVDAVSQNAAFLEQTLS | |
| | STIKQDDFTARLFDIHKQVLKEGIAQTVFLGLNRSDYMFQRSADGSP | |
| | ALKQIEINTISASFGGLASRTPAVHRHVLSVLSKTKEAGKILSNNPSK | |
| | GLALGIAKAWELYGSPNALVLLIAQEKERNIFDQRAIENELLARNIH | |
| | VIRRTFEDISEKGSLDQDRRLFVDGQEIAVVYFRDGYMPRQYSLQN | |
| | WEARLLLERSHAAKCPDIATQLAGTKKVQQELSRPGMLEMLLPGQ | |
| | PEAVARLRATFAGLYSLDVGEEGDQAIAEALAAPSRFVLKPQREGG | |
| | GNNLYGEEMVQALKQLKDSEERASYILMEKIEPEPFENCLLRPGSPA | |
| | RVVQCISELGIFGVYVRQEKTLVMNKHVGHLLRTKAIEHADGGVA | |
| | AGVAVLDNPYPV | |
| 160 | MGQREMWRLMSRFNAFKRTNTILHHLRMSKHTDAAEEVLLEKKG | HIBCH |
| | CTGVITLNRPKFLNALTLNMIRQIYPQLKKWEQDPETFLIIIKGAGGK | |
| | AFCAGGDIRVISEAEKAKQKIAPVFFREEYMLNNAVGSCQKPYVALI | |
| | HGITMGGGVGLSVHGQFRVATEKCLFAMPETAIGLFPDVGGGYFLP | |
| | RLQGKLGYFLALTGFRLKGRDVYRAGIATHFVDSEKLAMLEEDLLA | |
| | LKSPSKENIASVLENYHTESKIDRDKSFILEEHMDKINSCFSANTVEEI | |
| | IENLQQDGSSFALEQLKVINKMSPTSLKITLRQLMEGSSKTLQEVLT | |
| | MEYRLSQACMRGHDFHEGVRAVLIDKDQSPKWKPADLKEVTEEDL | |
| | NNHFKSLGSSDLKF | |
| 161 | MAGYLRVVRSLCRASGSRPAWAPAALTAPTSQEQPRRHYADKRIK | IDH2 |
| | VAKPVVEMDGDEMTRIIWQFIKEKLILPHVDIQLKYFDLGLPNRDQT | |
| | DDQVTIDSALATQKYSVAVKCATITPDEARVEEFKLKKMWKSPNG | |
| | TIRNILGGTVFREPIICKNIPRLVPGWTKPITIGRHAHGDQYKATDFV | |
| | ADRAGTFKMVFTPKDGSGVKEWEVYNFPAGGVGMGMYNTDESIS | |
| | GFAHSCFQYAIQKKWPLYMSTKNTILKAYDGRFKDIFQEIFDKHYK | |
| | TDFDKNKIWYEHRLIDDMVAQVLKSSGGFVWACKNYDGDVQSDIL | |
| | AQGFGSLGLMTSVLVCPDGKTIEAEAAHGTVTRHYREHQKGRPTST | |
| | NPIASIFAWTRGLEHRGKLDGNQDLIRFAQMLEKVCVETVESGAMT | |
| | KDLAGCIHGLSNVKLNEHFLNTTDFLDTIKSNLDRALGRQ | |
| 162 | MVPALRYLVGACGRARGLFAGGSPGACGFASGRPRPLCGGSRSAST | L2HGDH |
| | SSFDIVIVGGGIVGLASARALILRHPSLSIGVLEKEKDLAVHQTGHNS | |
| | GVIHSGIYYKPESLKAKLCVQGAALLYEYCQQKGISYKQCGKLIVA | |
| | VEQEEIPRLQALYEKGLQNGVPGLRLIQQEDIKKKEPYCRGLMAIDC | |
| | PHTGIVDYRQVALSFAQDFQEAGGSVLTNFEVKGIEMAKESPSRSID | |
| | GMQYPIVIKNTKGEEIRCQYVVTCAGLYSDRISELSGCTPDPRIVPFR | |
| | GDYLLLKPEKCYLVKGNIYPVPDSRFPFLGVHFTPRMDGSIWLGPN | |
| | AVLAFKREGYRPFDFSATDVMDIIINSGLIKLASQNFSYGVTEMYKA | |
| | CFLGATVKYLQKFIPEITISDILRGPAGVRAQALDRDGNLVEDFVFD | |
| | AGVGDIGNRILHVRNAPSPAATSSIAISGMIADEVQQRFEL | |
| 163 | MRGFGPGLTARRLLPLRLPPRPPGPRLASGQAAGALERAMDELLRR | MLYCD |
| | AVPPTPAYELREKTPAPAEGQCADFVSFYGGLAETAQRAELLGRLA | |
| | RGFGVDHGQVAEQSAGVLHLRQQQREAAVLLQAEDRLRYALVPR | |
| | YRGLFHHISKLDGGVRFLVQLRADLLEAQALKLVEGPDVREMNGV | |
| | LKGMLSEWFSSGFLNLERVTWHSPCEVLQKISEAEAVHPVKNWMD | |
| | MKRRVGPYRRCYFFSHCSTPGEPLVVLHVALTGDISSNIQAIVKEHP | |
| | PSETEEKNKITAAIFYSISLTQQGLQG | |
| | VELGTFLIKRVVKELQREFPHLGVFSSLSPIPGFTKWLLGLLNSQTKE | |
| | HGRNELFTDSECKEISEITGGPINETLKLLLSSSEWVQSEKLVRALQT | |
| | PLMRLCAWYLYGEKHRGYALNPVANFHLQNGAVLWRINWMADV | |
| | SLRGITGSCGLMANYRYFLEETGPNSTSYLGSKIIKASEQVLSLVAQF | |
| | QKNSKL | |
| 164 | MVVGAFPMAKLLYLGIRQVSKPLANRIKEAARRSEFFKTYICLPPAQ | OPA3 |
| | LYHWVEMRTKMRIMGFRGTVIKPLNEEAAAELGAELLGEATIFIVG | |
| | GGCLVLEYWRHQAQQRHKEEEQRAAWNALRDEVGHLALALEALQ | |
| | AQVQAAPPQGALEELRTELQEVRAQLCNPGRSASHAVPASKK | |
| 165 | MGSPEGRFHFAIDRGGTFTDVFAQCPGGHVRVLKLLSEDPANYADA | OPLAH |
| | PTEGIRRILEQEAGMLLPRDQPLDSSHIASIRMGTTVATNALLERKGE | |
| | RVALLVTRGFRDLLHIGTQARGDLFDLAVPMPEVLYEEVLEVDERV | |
| | VLHRGEAGTGTPVKGRTGDLLEVQQPVDLGALRGKLEGLLSRGIRS | |
| | LAVVLMHSYTWAQHEQQVGVLARELGFTHVSLSSEAMPMVRIVPR | |
| | GHTACADAYLTPAIQRYVQGFCRGFQGQLKDVQVLFMRSDGGLAP | |
| | MDTFSGSSAVLSGPAGGVVGYSATTYQQEGGQPVIGFDMGGTSTD | |
| | VSRYAGEFEHVFEASTAGVTLQAPQLDINTVAAGGGSRLFFRSGLF | |
| | VVGPESAGAHPGPACYRKGGPVTVTDANLVLGRLLPASFPCIFGPG | |
| | ENQPLSPEASRKALEAVATEVNSFLTNGPCPASPLSLEEVAMGFVRV | |
| | ANEAMCRPIRALTQARGHDPSAHVLACFGGAGGQHACAIARALGM | |
| | DTVHIHRHSGLLSALGLALADVVHEAQEPCSLLYAPETFVQLDQRL | |
| | SRLEEQCVDALQAQGFPRSQISTESFLHLRYQGTDCALMVSAHQHP | |
| | ATA | |
| | RSPRAGDFGAAFVERYMREFGFVIPERPVVVDDVRVRGTGRSGLRL | |
| | EDAPKAQTGPPRVDKMTQCYFEGGYQETPVYLLAELGYGHKLHGP | |
| | CLIIDSNSTILVEPGCQAEVTKTGDICISVGAEVPGTVGPQLDPIQLSIF | |
| | SHRFMSIAEQMGRILQRTAISTNIKERLDFSCALFGPDGGLVSNAPHI | |
| | PVHLGAMQETVQFQIQHLGADLHPGDVLLSNHPSAGGSHLPDLTVI | |
| | TPVFWPGQTRPVFYVASRGHHADIGGITPGSMPPHSTMLQQEGAVF | |
| | LSFKLVQGGVFQEEAVTEALRAPGKVPNCSGTRNLHDNLSDLRAQ | |
| | VAANQKGIQLVGELIGQYGLDVVQAYMGHIQANAELAVRDMLRAF | |
| | GTSRQARGLPLEVSSEDHMDDGSPIRLRVQISLSQGSAVFDFSGTGP | |
| | EVFGNLNAPRAVTLSALIYCLRCLVGRDIPLNQGCLAPVRVVIPRGSI | |
| | LDPSPEAAVVGGNVLTSQRVVDVILGAFGACAASQGCMNNVTLGN | |
| | AHMGYYETVAGGAGAGPSWHGRSGVHSHMTNTRITDPEILESRYP | |
| | VILRRFELRRGSGGRGRFRGGDGVTRELLFREEALLSVLTERRAFRP | |
| | YGLHGGEPGARGLNLLIRKNGRTVNLGGKTSVTVYPGDVFCLHTPG | |
| | GGGYGDPEDPAPPPGSPPQALAFPEHGSVYEYRRAQEAV | |
| 166 | MAALKLLSSGLRLCASARGSGATWYKGCVCSFSTSAHRHTKFYTD | OXCT1 |
| | PVEAVKDIPDGATVLVGGFGLCGIPENLIDALLKTGVKGLTAVSNN | |
| | AGVDNFGLGLLLRSKQIKRMVSSYVGENAEFERQYLSGELEVELTP | |
| | QGTLAERIRAGGAGVPAFYTPTGYGTLVQEGGSPIKYNKDGSVAIA | |
| | SKPREVREFNGQHFILEEAITGDFALVKAWKADRAGNVIFRKSARN | |
| | FNLPMCKAAETTVVEVEEIVDIGAFAPEDIHIPQIYVHRLIKGEKYEK | |
| | RIERLSIRKEGDGEAKSAKPGDDVRERIIKRAALEFEDGMYANLGIGI | |
| | PLLASNFISPNITVHLQSENGVLGLGPYPRQHEADADLINAGKETVTI | |
| | LPGASFFSSDESFAMIRGGHVDLTMLGAMQVSKYGDLANWMIPGK | |
| | MVKGMGGAMDLVSSAKTKVVVTMEHSAKGNAHKIMEKCTLPLTG | |
| | KQCVNRIITEKAVFDVDKKKGLTLIELWEGLTVDDVQKSTGCDFAV | |
| | SPKLMPMQQIAN | |
| 167 | MSRLLWRKVAGATVGPGPVPAPGRWVSSSVPASDPSDGQRRRQQQ | POLG |
| | QQQQQQQQQQPQQPQVLSSEGGQLRHNPLDIQMLSRGLHEQIFGQG | |
| | GEMPGEAAVRRSVEHLQKHGLWGQPAVPLPDVELRLPPLYGDNLD | |
| | QHFRLLAQKQSLPYLEAANLLLQAQLPPKPPAWAWAEGWTRYGPE | |
| | GEAVPVAIPEERALVFDVEVCLAEGTCPTLAVAISPSAWYSWCSQR | |
| | LVEERYSWTSQLSPADLIPLEVPTGASSPTQRDWQEQLVVGHNVSF | |
| | DRAHIREQYLIQGSRMRFLDTMSMHMAISGLSSFQRSLWIAAKQGK | |
| | HKVQPPTKQGQKSQRKARRGPAISSWDWLDISSVNSLAEVHRLYV | |
| | GGPPLEKEPRELFVKGTMKDIRENFQDLMQYCAQDVWATHEVFQQ | |
| | QLPLFLERCPHPVTLAGMLEMGVSYLPVNQNWERYLAEAQGTYEE | |
| | LQREMKKSLMDLANDACQLLSGERYKEDPWLWDLEWDLQEFKQK | |
| | KAKKVKKEPATASKLPIEGAGAPGDPMDQEDLGPCSEEEEFQQDV | |
| | MARACLQKLKGTTELLPKRPQHLPGHPGWYRKLCPRLDDPAWTPG | |
| | PSLLSLQMRVTPKLMALTWDGFPLHYSERHGWGYLVPGRRDNLAK | |
| | LPTGTTLESAGVVCPYRAIESLYRKHCLEQGKQQLMPQEAGLAEEF | |
| | LLTDNSAIWQTVEELDYLEVEAEAKMENLRAAVPGQPLALTARGG | |
| | PKDTQPSYHHGNGPYNDVDIPGCWFFKLPHKDGNSCNVGSPFAKDF | |
| | LPKMEDGTLQAGPGGASGPRALEINKMISFWRNAHKRISSQMVVW | |
| | LPRSALPRAVIRHPDYDEEGLYGAILPQVVTAGTITRRAVEPTWLTA | |
| | SNARPDRVGSELKAMVQAPPGYTLVGADVDSQELWIAAVLGDAHF | |
| | AGMHGCTAFGWMTLQGRKSRGTDLHSKTATTVGISREHAKIFNYG | |
| | RIYGAGQPFAERLLMQFNHRLTQQEAAEKAQQMYAATKGLRWYR | |
| | LSDEGEWLVRELNLPVDRTEGGWISLQDLRKVQRETARKSQWKKW | |
| | EVVAERAWKGGTESEMFNKLESIATSDIPRTPVLGCCISRALEPSAV | |
| | QEEFMTSRVNWVVQSSAVDYLHLMLVAMKWLFEEFAIDGRFCISIH | |
| | DEVRYLVREEDRYRAALALQITNLLTRCMFAYKLGLNDLPQSVAFF | |
| | SAVDIDRCLRKEVTMDCKTPSNPTGMERRYGIPQGEALDIYQIIELT | |
| | KGSLEKRSQPGP | |
| 168 | MSTAALITLVRSGGNQVRRRVLLSSRLLQDDRRVTPTCHSSTSEPRC | PPM1K |
| | SRFDPDGSGSPATWDNFGIWDNRIDEPILLPPSIKYGKPIPKISLENVG | |
| | CASQIGKRKENEDRFDFAQLTDEVLYFAVYDGHGGPAAADFCHTH | |
| | MEKCIMDLLPKEKNLETLLTLAFLEIDKAFSSHARLSADATLLTSGT | |
| | TATVALLRDGIELVVASVGDSRAILCRKGKPMKLTIDHTPERKDEKE | |
| | RIKKCGGFVAWNSLGQPHVNGRLAMTRSIGDLDLKTSGVIAEPETK | |
| | RIKLHHADDSFLVLTTDGINFMVNSQEICDFVNQCHDPNEAAHAVT | |
| | EQAIQYGTEDNSTAVVVPFGAWGKYKNSEINFSFSRSFASSGRWA | |
| 169 | MSLAAYCVICCRRIGTSTSPPKSGTHWRDIRNIIKFTGSLILGGSLFLT | SERAC1 |
| | YEVLALKKAVTLDTQVVEREKMKSYIYVHTVSLDKGENHGIAWQA | |
| | RKELHKAVRKVLATSAKILRNPFADPFSTVDIEDHECAVWLLLRKS | |
| | KSDDKTTRLEAVREMSETHHWHDYQYRIIAQACDPKTLIGLARSEE | |
| | SDLRFFLLPPPLPSLKEDSSTEEELRQLLASLPQTELDECIQYFTSLAL | |
| | SESSQ | |
| | SLAAQKGGLWCFGGNGLPYAESFGEVPSATVEMFCLEAIVKHSEIST | |
| | HCDKIEANGGLQLLQRLYRLHKDCPKVQRNIMRVIGNMALNEHLH | |
| | SSIVRSGWVSIMAEAMKSPHIMESSHAARILANLDRETVQEKYQDG | |
| | VYVLHPQYRTSQPIKADVLFIHGLMGAAFKTWRQQDSEQAVIEKPM | |
| | EDEDRYTTCWPKTWLAKDCPALRIISVEYDTSLSDWRARCPMERKS | |
| | IAFRSNELLRKLRAAGVGDRPVVWISHSMGGLLVKKMLLEASTKPE | |
| | MSTVINNTRGIIFYSVPHHGSRLAEYSVNIRYLLFPSLEVKELSKDSP | |
| | ALKTLQDDFLEFAKDKNFQVLNFVETLPTYIGSMIKLHVVPVESADL | |
| | GIGDLIPVDVNHLNICKPKKKDAFLYQRTLQFIREALAKDLEN | |
| 170 | MPAPRAPRALAAAAPASGKAKLTHPGKAILAGGLAGGIEICITFPTE | SLC25A1 |
| | YVKTQLQLDERSHPPRYRGIGDCVRQTVRSHGVLGLYRGLSSLLYG | |
| | SIPKAAVRFGMFEFLSNHMRDAQGRLDSTRGLLCGLGAGVAEAVV | |
| | VVCPMETIKVKFIHDQTSPNPKYRGFFHGVREIVREQGLKGTYQGLT | |
| | ATVLKQGSNQAIRFFVMTSLRNWYRGDNPNKPMNPLITGVFGAIAG | |
| | AASVFGNTPLDVIKTRMQGLEAHKYRNTWDCGLQILKKEGLKAFY | |
| | KGTVPRLGRVCLDVAIVFVIYDEV | |
| | VKLLNKVWKTD | |
| 171 | MAASMFYGRLVAVATLRNHRPRTAQRAAAQVLGSSGLFNNHGLQ | SUCLA2 |
| | VQQQQQRNLSLHEYMSMELLQEAGVSVPKGYVAKSPDEAYAIAKK | |
| | LGSKDVVIKAQVLAGGRGKGTFESGLKGGVKIVFSPEEAKAVSSQM | |
| | IGKKLFTKQTGEKGRICNQVLVCERKYPRREYYFAITMERSFQGPVL | |
| | IGSSHGGVNIEDVAAESPEAIIKEPIDIEEGIKKEQALQLAQKMGFPPN | |
| | IVESAAENMVKLYSLFLKYDATMIEINPMVEDSDGAVLCMDAKINF | |
| | DSNSAYRQKKIFDLQDWTQEDERDKDAAKANLNYIGLDGNIGCLV | |
| | NGAGLAMATMDIIKLHGGTPANFLDVGGGATVHQVTEAFKLITSDK | |
| | KVLAILVNIFGGIMRCDVIAQGIVMAVKDLEIKIPVVVRLQGTRVDD | |
| | AKALIADSGLKILACDDLDEAARMVVKLSEIVTLAKQAHVDVKFQL | |
| | PI | |
| 172 | MTATLAAAADIATMVSGSSGLAAARLLSRSFLLPQNGIRHCSYTAS | SUCLG1 |
| | RQHLYVDKNTKIICQGFTGKQGTFHSQQALEYGTKLVGGTTPGKGG | |
| | QTHLGLPVFNTVKEAKEQTGATASVIYVPPPFAAAAINEAIEAEIPLV | |
| | VCITEGIPQQDMVRVKHKLLRQEKTRLIGPNCPGVINPGECKIGIMP | |
| | GHIHKKGRIGIVSRSGTLTYEAVHQTTQVGLGQSLCVGIGGDPFNGT | |
| | DFIDCLEIFLNDSATEGIILIGEIGGNAEENAAEFLKQHNSGPNSKPVV | |
| | SFIAGLTAPPGRRMGHAGAIIAGGKGGAKEKISALQSAGVVVSMSP | |
| | AQLGTTIYKEFEKRKML | |
| 173 | MPLHVKWPFPAVPPLTWTLASSVVMGLVGTYSCFWTKYMNHLTV | TAZ |
| | HNREVLYELIEKRGPATPLITVSNHQSCMDDPHLWGILKLRHIWNLK | |
| | LMRWTPAAADICFTKELHSHFFSLGKCVPVCRGAEFFQAENEGKGV | |
| | LDTGRHMPGAGKRREKGDGVYQKGMDFILEKLNHGDWVHIFPEG | |
| | KVNMSSEFLRFKWGIGRLIAECHLNPIILPLWHVGMNDVLPNSPPYF | |
| | PRFGQKITVLIGKPFSALPVLERLRAENKSAVEMRKALTDFIQEEFQ | |
| | HLKTQAEQLHNHLQPGR | |
| 174 | MTVFFKTLRNHWKKTTAGLCLLTWGGHWLYGKHCDNLLRRAACQ | AGK |
| | EAQVFGNQLIPPNAQVKKATVFLNPAACKGKARTLFEKNAAPILHL | |
| | SGMDVTIVKTDYEGQAKKLLELMENTDVIIVAGGDGTLQEVVTGV | |
| | LRRTDEATFSKIPIGFIPLGETSSLSHTLFAESGNKVQHITDATLAIVK | |
| | GETVPLDVLQIKGEKEQPVFAMTGLRWGSFRDAGVKVSKYWYLGP | |
| | LKIKAAHFFSTLKEWPQTHQASISYTGPTERPPNEPEETPVQRPSLYR | |
| | RILRRLASYWAQPQDALSQEVSPEVWKDVQLSTIELSITTRNNQLDP | |
| | TSKEDFLNICIEPDTISKGDFITIGSRKVRNPKLHVEGTECLQASQCTL | |
| | LIPEGAGGSFSIDSEEYEAMPVEVKLLPRKLQFFCDPRKREQMLTSPT | |
| | Q | |
| 175 | MLGSLVLRRKALAPRLLLRLLRSPTLRGHGGASGRNVTTGSLGEPQ | CLPB |
| | WLRVATGGRPGTSPALFSGRGAATGGRQGGRFDTKCLAAATWGRL | |
| | PGPEETLPGQDSWNGVPSRAGLGMCALAAALVVHCYSKSPSNKDA | |
| | ALLEAARANNMQEVSRLLSEGADVNAKHRLGWTALMVAAINRNN | |
| | SVVQVLLAAGADPNLGDDFSSVYKTAKEQGIHSLEDGGQDGASRHI | |
| | TNQWTSALEFRRWLGLPAGVLITREDDFNNRLNNRASFKGCTALH | |
| | YAVLADDYRTVKELLDGGANPLQRNEMGHTPLDYAREGEVMKLL | |
| | RTSEAKYQEKQRKREAEERRRFPLEQRLKEHIIGQESAIATVGAA | |
| | IRRKENGWYDEEHPLVFLFLGSSGIGKTELAKQTAKYMHKDAKKG | |
| | FIRLDMSEFQERHEVAKFIGSPPGYVGHEEGGQLTKKLKQCPNAVV | |
| | LFDEVDKAHPDVLTIMLQLFDEGRLTDGKGKTIDCKDAIFIMTSNVA | |
| | SDEIAQHALQLRQEALEMSRNRIAENLGDVQISDKITISKNFKENVIR | |
| | PILKAHFRRDEFLGRINEIVYFLPFCHSELIQLVNKELNFWAKRAKQR | |
| | HNITLLWDREVADVLVDGYNVHYGARSIKHEVERRVVNQLAAAYE | |
| | QDLLPGGCTLRITVEDSDKQLLKSPELPSPQAEKRLPKLRLEIIDKDS | |
| | KTRRLDIRAPLHPEKVCNTI | |
| 176 | MLFLALGSPWAVELPLCGRRTALCAAAALRGPRASVSRASSSSGPS | TMEM70 |
| | GPVAGWSTGPSGAARLLRRPGRAQIPVYWEGYVRFLNTPSDKSEDG | |
| | RLIYTGNMARAVFGVKCFSYSTSLIGLTFLPYIFTQNNAISESVPLPIQ | |
| | IIFYGIMGSFTVITPVLLHFITKGYVIRLYHEATTDTYKAITYNAMLA | |
| | ETSTVFHQNDVKIPDAKHVFTTFYAKTKSLLVNPVLFPNREDYIHLM | |
| | GYDKEEFILYMEETSEEKRHKDDK | |
| 177 | MLSQVYRCGFQPFNQHLLPWVKCTTVFRSHCIQPSVIRHVRSWSNIP | ALDH18A1 |
| | FITVPLSRTHGKSFAHRSELKHAKRIVVKLGSAVVTRGDECGLALGR | |
| | LASIVEQVSVLQNQGREMMLVTSGAVAFGKQRLRHEILLSQSVRQA | |
| | LHSGQNQLKEMAIPVLEARACAAAGQSGLMALYEAMFTQYSICAA | |
| | QILVTNLDFHDEQKRRNLNGTLHELLRMNIVPIVNTNDAVVPPAEP | |
| | NSDLQGVNVISVKDNDSLAARLAVEMKTDLLIVLSDVEGLFDSPPG | |
| | SDDAKLIDIFYPGDQQSVTFGTKSRVGMGGMEAKVKAALWALQGG | |
| | TSVVIANGTHPKVSGHVITDIVEGKKVGTFFSEVKPAGPTVEQQGE | |
| | MARSGGRMLATLEPEQRAEIIHHLADLLTDQRDEILLANKKDLEEA | |
| | EGRLAAPLLKRLSLSTSKLNSLAIGLRQIAASSQDSVGRVLRRTRIAK | |
| | NLELEQVTVPIGVLLVIFESRPDCLPQVAALAIASGNGLLLKGGKEA | |
| | AHSNRILHLLTQEALSIHGVKEAVQLVNTREEVEDLCRLDKMIDLIIP | |
| | RGSSQLVRDIQKAAKGIPVMGHSEGICHMYVDSEASVDKVTRLVRD | |
| | SKCEYPAACNALETLLIHRDLLRTPLFDQIIDMLRVEQVKIHAGPKF | |
| | ASYLTFSPSEVKSLRTEYGDLELCIEVVDNVQDAIDHIHKYGSSHTD | |
| | VIVTEDENTAEFFLQHVDSACVFWNASTRFSDGYRFGLGAEVGISTS | |
| | RIHARGPVGLEGLLTTKWLLRGKDHVVSDFSEHGSLKYLHENLPIP | |
| | QRNTN | |
| 178 | MFSKLAHLQRFAVLSRGVHSSVASATSVATKKTVQGPPTSDDIFERE | OAT |
| | YKYGAHNYHPLPVALERGKGIYLWDVEGRKYFDFLSSYSAVNQGH | |
| | CHPKIVNALKSQVDKLTLTSRAFYNNVLGEYEEYITKLFNYHKVLP | |
| | MNTGVEAGETACKLARKWGYTVKGIQKYKAKIVFAAGNFWGRTL | |
| | SAISSSTDPTSYDGFGPFMPGFDIIPYNDLPALERALQDPNVAAFMVE | |
| | PIQGEAGVVVPDPGYLMGVRELCTRHQVLFIADEIQTGLARTGRWL | |
| | AVDYENVRPDIVLLGKALSGGLYPVSAVLCDDDIMLTIKPGEHGST | |
| | YGGNPLGCRVAIAALEVLEEENLAENADKLGIILRNELMKLPSDVVT | |
| | AVRGKGLLNAIVIKETKDWDAWKVCLRLRDNGLLAKPTHGDIIRFA | |
| | PPLVIKEDELRESIEIINKTILSF | |
| 179 | MLGRNTWKTSAFSFLVEQMWAPLWSRSMRPGRWCSQRSCAWQTS | CA5A |
| | NNTLHPLWTVPVSVPGGTRQSPINIQWRDSVYDPQLKPLRVSYEAA | |
| | SCLYIWNTGYLFQVEFDDATEASGISGGPLENHYRLKQFHFHWGAV | |
| | NEGGSEHTVDGHAYPAELHLVHWNSVKYQNYKEAVVGENGLAVI | |
| | GVFLKLGAHHQTLQRLVDILPEIKHKDARAAMRPFDPSTLLPTCWD | |
| | YWTYAGSLTTPPLTESVTWIIQKEPVEVAPSQLSAFRTLLFSALGEEE | |
| | KMMVNNYRPLQPLMNRKVWASFQATNEGTRS | |
| 180 | MYRYLGEALLLSRAGPAALGSASADSAALLGWARGQPAAAPQPGL | GLUD1 |
| | ALAARRHYSEAVADREDDPNFFKMVEGFFDRGASIVEDKLVEDLRT | |
| | RESEEQKRNRVRGILRIIKPCNHVLSLSFPIRRDDGSWEVIEGYRAQH | |
| | SQHRTPCKGGIRYSTDVSVDEVKALASLMTYKCAVVDVPFGGAKA | |
| | GVKINPKNYTDNELEKITRRFTMELAKKGFIGPGIDVPAPDMSTGER | |
| | EMSWIADTYASTIGHYDINAHACVTGKPISQGGIHGRISATGRGVFH | |
| | GIENFINEASYMSILGMTPGFG | |
| | DKTFVVQGFGNVGLHSMRYLHRFGAKCIAVGESDGSIWNPDGIDPK | |
| | ELEDFKLQHGSILGFPKAKPYEGSILEADCDILIPAASEKQLTKSNAP | |
| | RVKAKIIAEGANGPTTPEADKIFLERNIMVIPDLYLNAGGVTVSYFE | |
| | WLKNLNHVSYGRLTFKYERDSNYHLLMSVQESLERKFGKHGGTIPI | |
| | VPTAEFQDRISGASEKDIVHSGLAYTMERSARQIMRTAMKYNLGLD | |
| | LRTAAYVNAIEKVFKVYNEAGVTFT | |
| 181 | MTTSASSHLNKGIKQVYMSLPQGEKVQAMYIWIDGTGEGLRCKTR | GLUL |
| | TLDSEPKCVEELPEWNFDGSSTLQSEGSNSDMYLVPAAMFRDPFRK | |
| | DPNKLVLCEVFKYNRRPAETNLRHTCKRIMDMVSNQHPWFGMEQE | |
| | YTLMGTDGHPFGWPSNGFPGPQGPYYCGVGADRAYGRDIVEAHYR | |
| | ACLYAGVKIAGTNAEVMPAQWEFQIGPCEGISMGDHLWVARFILH | |
| | RVCEDFGVIATFDPKPIPGNWNGAGCHTNFSTKAMREENGLKYIEE | |
| | AIEKLSKRHQYHIRAYDPKGGLDNARRLTGFHETSNINDFSAGVAN | |
| | RSASIRIPRTVGQEKKGYFEDRRPSANCDPFSVTEALIRTCLLNETGD | |
| | EPFQYKN | |
| 182 | MAVARAALGPLVTGLYDVQAFKFGDFVLKSGLSSPIYIDLRGIVSRP | UMPS |
| | RLLSQVADILFQTAQNAGISFDTVCGVPYTALPLATVICSTNQIPMLI | |
| | RRKETKDYGTKRLVEGTINPGETCLIIEDVVTSGSSVLETVEVLQKE | |
| | GLKVTDAIVLLDREQGGKDKLQAHGIRLHSVCTLSKMLEILEQQKK | |
| | VDAETVGRVKRFIQENVFVAANHNGSPLSIKEAPKELSFGARAELPR | |
| | IHPVA | |
| | SKLLRLMQKKETNLCLSADVSLARELLQLADALGPSICMLKTHVDI | |
| | LNDFTLDVMKELITLAKCHEFLIFEDRKFADIGNTVKKQYEGGIFKIA | |
| | SWADLVNAHVVPGSGVVKGLQEVGLPLHRGCLLIAEMSSTGSLAT | |
| | GDYTRAAVRMAEEHSEFVVGFISGSRVSMKPEFLHLTPGVQLEAGG | |
| | DNLGQQYNSPQEVIGKRGSDIIIVGRGIISAADRLEAAEMYRKAAWE | |
| | AYLSRLGV | |
| 183 | MRDYDEVTAFLGEWGPFQRLIFFLLSASIIPNGFTGLSSVFLIATPEHR | SLC22A5 |
| | CRVPDAANLSSAWRNHTVPLRLRDGREVPHSCRRYRLATIANFSAL | |
| | GLEPGRDVDLGQLEQESCLDGWEFSQDVYLSTIVTEWNLVCEDDW | |
| | KAPLTISLFFVGVLLGSFISGQLSDRFGRKNVLFVTMGMQTGFSFLQI | |
| | FSKNFEMFVVLFVLVGMGQISNYVAAFVLGTEILGKSVRIIFSTLGV | |
| | CIFYAFGYMVLPLFAYFIRDWRMLLVALTMPGVLCVALWWFIPESP | |
| | RWLISQGRFEEAEVIIRKAAKANGIVVPSTIFDPSELQDLSSKKQQSH | |
| | NILDLLRTWNIRMVTIMSIMLWMTISVGYFGLSLDTPNLHGDIFVNC | |
| | FLSAMVEVPAYVLAWLLLQYLPRRYSMATALFLGGSVLLFMQLVP | |
| | PDLYYLATVLVMVGKFGVTAAFSMVYVYTAELYPTVVRNMGVGV | |
| | SSTASRLGSILSPYFVYLGAYDRFLPYILMGSLTILTAILTLFLPESFGT | |
| | PLPDTIDQMLRVKGMKHRKTPSHTR | |
| | MLKDGQERPTILKSTAF | |
| 184 | MAEAHQAVAFQFTVTPDGIDLRLSHEALRQIYLSGLHSWKKKFIRF | CPT1A |
| | KNGIITGVYPASPSSWLIVVVGVMTTMYAKIDPSLGIIAKINRTLETA | |
| | NCMSSQTKNVVSGVLFGTGLWVALIVTMRYSLKVLLSYHGWMFTE | |
| | HGKMSRATKIWMGMVKIFSGRKPMLYSFQTSLPRLPVPAVKDTVN | |
| | RYLQSVRPLMKEEDFKRMTALAQDFAVGLGPRLQWYLKLKSWWA | |
| | TNYVSDWWEEYIYLRGRGPLMVNSNYYAMDLLYILPTHIQAARAG | |
| | NAIHAILLYRRKLDREEIKPIRLLGSTIPLCSAQWERMFNTSRIPGEET | |
| | DTIQHMRDSKHIVVYHRGRYFKVWLYHDGRLLKPREMEQQMQRIL | |
| | DNTSEPQPGEARLAALTAGDRVPWARCRQAYFGRGKNKQSLDAVE | |
| | KAAFFVTLDETEEGYRSEDPDTSMDSYAKSLLHGRCYDRWFDKSFT | |
| | FVVFKNGKMGLNAEHSWADAPIVAHLWEYVMSIDSLQLGYAEDG | |
| | HCKGDINPNIPYPTRLQWDIPGECQEVIETSLNTANLLANDVDFHSFP | |
| | FVAFGKGIIKKCRTSPDAFVQLALQLAHYKDMGKFCLTYEASMTRL | |
| | FREGRTETVRSCTTESCDFVRAMVDPAQTVEQRLKLFKLASEKHQH | |
| | MYRLAMTGSGIDRHLFCLYVVSKYLAVESPFLKEVLSEPWRLSTSQ | |
| | TPQQQVELFDLENNPEYVSSGGGFGPVADDGYGVSYILVGENLINF | |
| | HISSKFSCPETDSHRFGRHLKEAMTDIITLFGLSSNSKK | |
| 185 | MVACRAIGILSRFSAFRILRSRGYICRNFTGSSALLTRTHINYGVKGD | HADHA |
| | VAVVRINSPNSKVNTLSKELHSEFSEVMNEIWASDQIRSAVLISSKPG | |
| | CFIAGADINMLAACKTLQEVTQLSQEAQRIVEKLEKSTKPIVAAING | |
| | SCLGGGLEVAISCQYRIATKDRKTVLGTPEVLLGALPGAGGTQRLP | |
| | KMVGVPAALDMMLTGRSIRADRAKKMGLVDQLVEPLGPGLKPPEE | |
| | RTIEYLEEVAITFAKGLADKKISPKRDKGLVEKLTAYAMTIPFVRQQ | |
| | VYKKVEEKVRKQTKGLYPAPLKIIDVVKTGIEQGSDAGYLCESQKF | |
| | GELVMTKESKALMGLYHGQVLCKKNKFGAPQKDVKHLAILGAGL | |
| | MGAGIAQVSVDKGLKTILKDATLTALDRGQQQVFKGLNDKVKKKA | |
| | LTSFERDSIFSNLTGQLDYQGFEKADMVIEAVFEDLSLKHRVLKEVE | |
| | AVIPDHCIFASNTSALPISEIAAVSKRPEKVIGMHYFSPVDKMQLLEII | |
| | TTEKTSKDTSASAVAVGLKQGKVIIVVK | |
| | DGPGFYTTRCLAPMMSEVIRILQEGVDPKKLDSLTTSFGFPVGAATL | |
| | VDEVGVDVAKHVAEDLGKVFGERFGGGNPELLTQMVSKGFLGRKS | |
| | GKGFYIYQEGVKRKDLNSDMDSILASLKLPPKSEVSSDEDIQFRLVT | |
| | RFVNEAVMCLQEGILATPAEGDIGAVFGLGFPPCLGGPFRFVDLYG | |
| | AQKIVDRLKKYEAAYGKQFTPCQLLADHANSPNKKFYQ | |
| 186 | MAFVTRQFMRSVSSSSTASASAKKIIVKHVTVIGGGLMGAGIAQVA | HADH |
| | AATGHTVVLVDQTEDILAKSKKGIEESLRKVAKKKFAENLKAGDEF | |
| | VEKTLSTIATSTDAASVVHSTDLVVEAIVENLKVKNELFKRLDKFAA | |
| | EHTIFASNTSSLQITSIANATTRQDRFAGLHFFNPVPVMKLVEVIKTP | |
| | MTSQKTFESLVDFSKALGKHPVSCKDTPGFIVNRLLVPYLMEAIRLY | |
| | ERGDASKEDIDTAMKLGAGYPMGPFELLDYVGLDTTKFIVDGWHE | |
| | MDAENPLHQPSPSLNKLVAENKFGKKTGEGFYKYK | |
| 187 | MAAPTLGRLVLTHLLVALFGMGSWAAVNGIWVELPVVVKDLPEG | SLC52A1 |
| | WSLPSYLSVVVALGNLGLLVVTLWRQLAPGKGEQVPIQVVQVLSV | |
| | VGTALLAPLWHHVAPVAGQLHSVAFLTLALVLAMACCTSNVTFLP | |
| | FLSHLPPPFLRSFFLGQGLSALLPCVLALVQGVGRLECPPAPTNGTSG | |
| | PPLDFPERFPASTFFWALTALLVTSAAAFRGLLLLLPSLPSVTTGGSG | |
| | PELQLGSPGAEEEEKEEEEALPLQEPPSQAAGTIPGPDPEAHQLFSAH | |
| | GAFLLGLMAFTSAVTNGVLPSVQSFSCLPYGRLAYHLAVVLGSAAN | |
| | PLACFLAMGVLCRSLAGLVGLSLLGMLFGAYLMALAILSPCPPLVG | |
| | TTAGVVLVVLSWVLCLCVFSYVKVAASSLLHGGGRPALLAAGVAI | |
| | QVGSLLGAGAMFPPTSIYHVFQSRKDCVDPCGP | |
| 188 | MAAPTPARPVLTHLLVALFGMGSWAAVNGIWVELPVVVKELPEG | SLC52A2 |
| | WSLPSYVSVLVALGNLGLLVVTLWRRLAPGKDEQVPIRVVQVLGM | |
| | VGTALLASLWHHVAPVAGQLHSVAFLALAFVLALACCASNVTFLP | |
| | FLSHLPPRFLRSFFLGQGLSALLPCVLALVQGVGRLECPPAPINGTPG | |
| | PPLDFLERFPASTFFWALTALLVASAAAFQGLLLLLPPPPSVPTGELG | |
| | SGLQVGAPGAEEEVEESSPLQEPPSQAAGTTPGPDPKAYQLLSARSA | |
| | CLLGLLAATNALTNGVLPAVQSFSCLPYGRLAYHLAVVLGSAANPL | |
| | ACFLAMGVLCRSLAGLGGLSLLGVFCGGYLMALAVLSPCPPLVGTS | |
| | AGVVLVVLSWVLCLGVFSYVKVAASSLLHGGGRPALLAAGVAIQV | |
| | GSLLGAVAMFPPTSIYHVFHSRKDCADPCDS | |
| 189 | MAFLMHLLVCVFGMGSWVTINGLWVELPLLVMELPEGWYLPSYLT | SLC52A3 |
| | VVIQLANIGPLLVTLLHHFRPSCLSEVPIIFTLLGVGTVTCIIFAFLWN | |
| | MTSWVLDGHHSIAFLVLTFFLALVDCTSSVTFLPFMSRLPTYYLTTF | |
| | FVGEGLSGLLPALVALAQGSGLTTCVNVTEISDSVPSPVPTRETDIAQ | |
| | GVPRALVSALPGMEAPLSHLESRYLPAHFSPLVFFLLLSIMMACCLV | |
| | AFFV | |
| | LQRQPRCWEASVEDLLNDQVTLHSIRPREENDLGPAGTVDSSQGQG | |
| | YLEEKAAPCCPAHLAFIYTLVAFVNALTNGMLPSVQTYSCLSYGPV | |
| | AYHLAATLSIVANPLASLVSMFLPNRSLLFLGVLSVLGTCFGGYNM | |
| | AMAVMSPCPLLQGHWGGEVLIVASWVLFSGCLSYVKVMLGVVLR | |
| | DLSRSALLWCGAAVQLGSLLGALLMFPLVNVLRLFSSADFCNLHCP | |
| | A | |
| 190 | MTILTYPFKNLPTASKWALRFSIRPLSCSSQLRAAPAVQTKTKKTLA | HADHB |
| | KPNIRNVVVVDGVRTPFLLSGTSYKDLMPHDLARAALTGLLHRTSV | |
| | PKEVVDYIIFGTVIQEVKTSNVAREAALGAGFSDKTPAHTVTMACIS | |
| | ANQAMTTGVGLIASGQCDVIVAGGVELMSDVPIRHSRKMRKLMLD | |
| | LNKAKSMGQRLSLISKFRFNFLAPELPAVSEFSTSETMGHSADRLAA | |
| | AFAVSRLEQDEYALRSHSLAKKAQDEGLLSDVVPFKVPGKDTVTK | |
| | DNGIRPSSLEQMAKLKPAFIKPY | |
| | GTVTAANSSFLTDGASAMLIMAEEKALAMGYKPKAYLRDFMYVSQ | |
| | DPKDQLLLGPTYATPKVLEKAGLTMNDIDAFEFHEAFSGQILANFK | |
| | AMDSDWFAENYMGRKTKVGLPPLEKFNNWGGSLSLGHPFGATGC | |
| | RLVMAAANRLRKEGGQYGLVAACAAGGQGHAMIVEAYPK | |
| 191 | MLRGRSLSVTSLGGLPQWEVEELPVEELLLFEVAWEVTNKVGGIYT | GYS2 |
| | VIQTKAKTTADEWGENYFLIGPYFEHNMKTQVEQCEPVNDAVRRA | |
| | VDAMNKHGCQVHFGRWLIEGSPYVVLFDIGYSAWNLDRWKGDLW | |
| | EACSVGIPYHDREANDMLIFGSLTAWFLKEVTDHADGKYVVAQFH | |
| | EWQAGIGLILSRARKLPIATIFTTHATLLGRYLCAANIDFYNHLDKFN | |
| | IDKEAGERQIYHRYCMERASVHCAHVFTTVSEITAIEAEHMLKRKP | |
| | DVVTPNGLNVKKFSAVHEFQNLHAMYKARIQDFVRGHFYGHLDFD | |
| | LEKTLFLFIAGRYEFSNKGADIFLESLSRLNFLLRMHKSDITVMVFFI | |
| | MPAKTNNFNVETLKGQAVRKQLWDVAHSVKEKFGKKLYDALLRG | |
| | EIPDLNDILDRDDLTIMKRAIFSTQRQSLPPVTTHNMIDDSTDPILSTI | |
| | RRIGLFNNRTDRVKVILHPEFLSSTSPLLPMDYEEFVRGCHLGVFPSY | |
| | YEPWGYTPAECTVMGIPSVTTNLSGFGCFMQEHVADPTAYGIYIVD | |
| | RRFRSPDDSCNQLTKFLYGFCKQSRRQRIIQRNRTERLSDLLDWRYL | |
| | GRYYQHARHLTLSRAFPDKFHVELTSPPTTEGFKYPRPSSVPPSPSGS | |
| | QASSPQSSDVEDEVEDERYDEEEEAERDRLNIKSPFSLSHVPHGKKK | |
| | LHGEYKN | |
| 192 | MAKPLTDQEKRRQISIRGIVGVENVAELKKSFNRHLHFTLVKDRNV | PYGL |
| | ATTRDYYFALAHTVRDHLVGRWIRTQQHYYDKCPKRVYYLSLEFY | |
| | MGRTLQNTMINLGLQNACDEAIYQLGLDIEELEEIEEDAGLGNGGL | |
| | GRLAACFLDSMATLGLAAYGYGIRYEYGIFNQKIRDGWQVEEADD | |
| | WLRYGNPWEKSRPEFMLPVHFYGKVEHTNTGTKWIDTQVVLALPY | |
| | DTPVPGYMNNTVNTMRLWSARAPNDFNLRDFNVGDYIQAVLDRN | |
| | LAENISRVLYPNDNFFEGKELRLKQEYFVVAATLQDIIRRFKASKFG | |
| | STRGAGTVFDAFPDQVAIQLNDTHPALAIPELMRIFVDIEKL | |
| | PWSKAWELTQKTFAYTNHTVLPEALERWPVDLVEKLLPRHLEIIYEI | |
| | NQKHLDRIVALFPKDVDRLRRMSLIEEEGSKRINMAHLCIVGSHAV | |
| | NGVAKIHSDIVKTKVFKDFSELEPDKFQNKTNGITPRRWLLLCNPGL | |
| | AELIAEKIGEDYVKDLSQLTKLHSFLGDDVFLRELAKVKQENKLKFS | |
| | QFLETEYKVKINPSSMFDVQVKRIHEYKRQLLNCLHVITMYNRIKK | |
| | DPKKLFVPRTVIIGGKAAPGYHMAKMIIKLITSVADVVNNDPMVGS | |
| | KLKVIFLENYRVSLAEKVIPATDLSEQISTAGTEASGTGNMKFMLNG | |
| | ALTIGTMDGANVEMAEEAGEENLFIFGMRIDDVAALDKKGYEAKE | |
| | YYEALPELKLVIDQIDNGFFSPKQPDLFKDIINMLFYHDRFKVFADY | |
| | EAYVKCQDKVSQLYMNPKAWNTMVLKNIAASGKFSSDRTIKEYAQ | |
| | NIWNVEPSDLKISLSNESNKVNGN | |
| 193 | MTEDKVTGTLVFTVITAVLGSFQFGYDIGVINAPQQVIISHYRHVLG | SLC2A2 |
| | VPLDDRKAINNYVINSTDELPTISYSMNPKPTPWAEEETVAAAQLIT | |
| | MLWSLSVSSFAVGGMTASFFGGWLGDTLGRIKAMLVANILSLVGA | |
| | LLMGFSKLGPSHILIIAGRSISGLYCGLISGLVPMYIGEIAPTALRGAL | |
| | GTFHQLAIVTGILISQIIGLEFILGNYDLWHILLGLSGVRAILQSLLLFF | |
| | CPESPRYLYIKLDEEVKAKQSLKRLRGYDDVTKDINEMRKEREEAS | |
| | SEQKVSIIQLFTNSSYRQPILVALMLHVAQQFSGINGIFYYSTSIFQTA | |
| | GISKPVYATIGVGAVNMVFTAVSVFLVEKAGRRSLFLIGMSGMFVC | |
| | AIFMSVGLVLLNKFSWMSYVSMIAIFLFVSFFEIGPGPIPWFMVAEFF | |
| | SQGPRPAALAIAAFSNWTCNFIVALCFQYIADFCGPYVFFLFAGVLL | |
| | AFTLFTFFKVPETKGKSFEEIAAEFQKKSGSAHRPKAAVEMKFLGAT | |
| | ETV | |
| 194 | MAASCLVLLALCLLLPLLLLGGWKRWRRGRAARHVVAVVLGDVG | ALG1 |
| | RSPRMQYHALSLAMHGFSVTLLGFCNSKPHDELLQNNRIQIVGLTE | |
| | LQSLAVGPRVFQYGVKVVLQAMYLLWKLMWREPGAYIFLQNPPG | |
| | LPSIAVCWFVGCLCGSKLVIDWHNYGYSIMGLVHGPNHPLVLLAK | |
| | WYEKFFGRLSHLNLCVTNAMREDLADNWHIRAVTVYDKPASFFKE | |
| | TPLDLQHRLFMKLGSMHSPFRARSEPEDPVTERSAFTERDAGSGLVT | |
| | RLRERPALLVSSTSWTEDEDFSILLAALEKFEQLTLDGHNLPSLVCVI | |
| | TGKGPLREYYSRLIHQKHFQHIQVCTPWLEAEDYPLLLGSADLGVC | |
| | LHTSSSGLDLPMKVVDMFGCCLPVCAVNFKCLHELVKHEENGLVF | |
| | EDSEELAAQLQMLFSNFPDPAGKLNQFRKNLRESQQLRWDESWVQ | |
| | TVLPLVMDT | |
| 195 | MAEEQGRERDSVPKPSVLFLHPDLGVGGAERLVLDAALALQARGC | ALG2 |
| | SVKIWTAHYDPGHCFAESRELPVRCAGDWLPRGLGWGGRGAAVC | |
| | AYVRMVFLALYVLFLADEEFDVVVCDQVSACIPVFRLARRRKKILF | |
| | YCHFPDLLLTKRDSFLKRLYRAPIDWIEEYTTGMADCILVNSQFTAA | |
| | VFKETFKSLSHIDPDVLYPSLNVTSFDSVVPEKLDDLVPKGKKFLLL | |
| | SINRYERKKNLTLALEALVQLRGRLTSQDWERVHLIVAGGYDERVL | |
| | ENVEHYQELKKMVQQSDLGQYVTFLRSFSDKQKISLLHSCTCVLYT | |
| | PSNEHFGIVPLEAMYMQCPVIAVNSGGPLESIDHSVTGFLCEPDPVH | |
| | FSEAIEKFIREPSLKATMGLAGRARVKEKFSPEAFTEQLYRYVTKLL | |
| | V | |
| 196 | MAAGLRKRGRSGSAAQAEGLCKQWLQRAWQERRLLLREPRYTLL | ALG3 |
| | VAACLCLAEVGITFWVIHRVAYTEIDWKAYMAEVEGVINGTYDYT | |
| | QLQGDTGPLVYPAGFVYIFMGLYYATSRGTDIRMAQNIFAVLYLAT | |
| | LLLVFLIYHQTCKVPPFVFFFMCCASYRVHSIFVLRLFNDPVAMVLL | |
| | FLSINLLLAQRWGWGCCFFSLAVSVKMNVLLFAPGLLFLLLTQFGF | |
| | RGALPKLGICAGLQVVLGLPFLLENPSGYLSRSFDLGRQFLFHWTVN | |
| | WRFLPEALFLHRAFHLALLTAHLTL | |
| | LLLFALCRWHRTGESILSLLRDPSKRKVPPQPLTPNQIVSTLFTSNFIG | |
| | ICFSRSLHYQFYVWYFHTLPYLLWAMPARWLTHLLRLLVLGLIELS | |
| | WNTYPSTSCSSAALHICHAVILLQLWLGPQPFPKSTQHSKKAH | |
| 197 | MEKWYLMTVVVLIGLTVRWTVSLNSYSGAGKPPMFGDYEAQRHW | ALG6 |
| | QEITFNLPVKQWYFNSSDNNLQYWGLDYPPLTAYHSLLCAYVAKFI | |
| | NPDWIALHTSRGYESQAHKLFMRTTVLIADLLIYIPAVVLYCCCLKE | |
| | ISTKKKIANALCILLYPGLILIDYGHFQYNSVSLGFALWGVLGISCDC | |
| | DLLGSLAFCLAINYKQMELYHALPFFCFLLGKCFKKGLKGKGFVLL | |
| | VKLACIVVASFVLCWLPFFTEREQTLQVLRRLFPVDRGLFEDKVANI | |
| | WCSFNVFLKIKDILPRHIQLIMSFCSTFLSLLPACIKLILQPSSKGFKFT | |
| | LVSCALSFFLFSFQVHEKSILLVSLPVCLVLSEIPFMSTWFLLVSTFSM | |
| | LPLLLKDELLMPSVVTTMAFFIACVTSFSIFEKTSEEELQLKSFSISVR | |
| | KYLPCFTFLSRIIQYLFLISVITMVLLTLMTVTLDPPQKLPDLFSVLVC | |
| | FVSCLNFLFFLVYFNIIIMWDSKSGRNQKKIS | |
| 198 | MAALTIATGTGNWFSALALGVTLLKCLLIPTYHSTDFEVHRNWLAI | ALG8 |
| | THSLPISQWYYEATSEWTLDYPPFFAWFEYILSHVAKYFDQEMLNV | |
| | HNLNYSSSRTLLFQRFSVIFMDVLFVYAVRECCKCIDGKKVGKELTE | |
| | KPKFILSVLLLWNFGLLIVDHIHFQYNGFLFGLMLLSIARLFQKRHM | |
| | EGAFLFAVLLHFKHIYLYVAPAYGVYLLRSYCFTANKPDGSIRWKS | |
| | FSFVRVISLGLVVFLVSALSLGPFLALNQLPQVFSRLFPFKRGLCHAY | |
| | WAPNFWALYNALDKVLSVIGLKLKFLDPNNIPKASMTSGLVQQFQ | |
| | HTVLPSVTPLATLICTLIAILPSIFCLWFKPQGPRGFLRCLTLCALSSF | |
| | MFGWHVHEKAILLAILPMSLLSVGKAGDASIFLILTTTGHYSLFPLLF | |
| | TAPELPIKILLMLLFTIYSISSLKTLFRKEKPLFNWMETFYLLGLGPLE | |
| | VCCEFVFPFTSWKVKYPFIPLLLTSVYCAVGITYAWFKLYVSVLIDS | |
| | AIGKTKKQ | |
| 199 | MASRGARQRLKGSGASSGDTAPAADKLRELLGSREAGGAEHRTEL | ALG9 |
| | SGNKAGQVWAPEGSTAFKCLLSARLCAALLSNISDCDETFNYWEPT | |
| | HYLIYGEGFQTWEYSPAYAIRSYAYLLLHAWPAAFHARILQTNKILV | |
| | FYFLRCLLAFVSCICELYFYKAVCKKFGLHVSRMMLAFLVLSTGMF | |
| | CSSSAFLPSSFCMYTTLIAMTGWYMDKTSIAVLGVAAGAILGWPFS | |
| | AALGLPIAFDLLVMKHRWKSFFHWSLMALILFLVPVVVIDSYYYGK | |
| | LVIAPLNIVLYNVFTPHGPDLYGT | |
| | EPWYFYLINGFLNFNVAFALALLVLPLTSLMEYLLQRFHVQNLGHP | |
| | YWLTLAPMYIWFIIFFIQPHKEERFLFPVYPLICLCGAVALSALQKCY | |
| | HFVFQRYRLEHYTVTSNWLALGTVFLFGLLSFSRSVALFRGYHGPL | |
| | DLYPEFYRIATDPTIHTVPEGRPVNVCVGKEWYRFPSSFLLPDNWQL | |
| | QFIPSEFRGQLPKPFAEGPLATRIVPTDMNDQNLEEPSRYIDISKCHY | |
| | LVDLDTMRETPREPKYSSNKEEWISLAYRPFLDASRSSKLLRAFYVP | |
| | FLSDQYTVYVNYTILKPRKAKQIRKKSGG | |
| 200 | MAAGERSWCLCKLLRFFYSLFFPGLIVCGTLCVCLVIVLWGIRLLLQ | ALG11 |
| | RKKKLVSTSKNGKNQMVIAFFHPYCNAGGGGERVLWCALRALQK | |
| | KYPEAVYVVYTGDVNVNGQQILEGAFRRFNIRLIHPVQFVFLRKRY | |
| | LVEDSLYPHFTLLGQSLGSIFLGWEALMQCVPDVYIDSMGYAFTLPL | |
| | FKYIGGCQVGSYVHYPTISTDMLSVVKNQNIGFNNAAFITRNPFLSK | |
| | VKLIYYYLFAHYGLVGSCSDVVMVNSSWTLNHILSLWKVGNCTNI | |
| | VYPPCDVQTFLDIPLHEKKMTPGHLLVSVGQFRPEKNHPLQIRAFAK | |
| | LLNKKMVESPPSLKLVLIGGCRNKDDELRVNQLRRLSEDLGVQEYV | |
| | EFKINIPFDELKNYLSEATIGLHTMWNEHFGIGVVECMAAGTIILAH | |
| | NSGGPKLDIVVPHEGDITGFLAESEEDYAETIAHILSMSAEKRLQIRK | |
| | SARASVSRFSDQEFEVTFLSSVEKLFK | |
| 201 | MAGKGSSGRRPLLLGLLVAVATVHLVICPYTKVEESFNLQATHDLL | ALG12 |
| | YHWQDLEQYDHLEFPGVVPRTFLGPVVIAVFSSPAVYVLSLLEMSK | |
| | FYSQLIVRGVLGLGVIFGLWTLQKEVRRHFGAMVATMFCWVTAM | |
| | QFHLMFYCTRTLPNVLALPVVLLALAAWLRHEWARFIWLSAFAIIV | |
| | FRVELCLFLGLLLLLALGNRKVSVVRALRHAVPAGILCLGLTVAVD | |
| | SYFWRQLTWPEGKVLWYNTVLNKSSNWGTSPLLWYFYSALPRGL | |
| | GCSLLFIPLGLVDRRTHAPTVLALGFMALYSLLPHKELRFIIYAFPML | |
| | NITAARGCSYLLNNYKKSWLYKAGSLLVIGHLVVNAAYSATALYV | |
| | SHFNYPGGVAMQRLHQLVPPQTDVLLHIDVAAAQTGVSRFLQVNS | |
| | AWRYDKREDVQPGTGMLAYTHILMEAAPGLLALYRDTHRVLASV | |
| | VGTTGVSLNLTQLPPFNVHLQTKLVLLERLPRPS | |
| 202 | MKCVFVTVGTTSFDDLIACVSAPDSLQKIESLGYNRLILQIGRGTVV | ALG13 |
| | PEPFSTESFTLDVYRYKDSLKEDIQKADLVISHAGAGSCLETLEKGK | |
| | PLVVVINEKLMNNHQLELAKQLHKEGHLFYCTCRVLTCPGQAKSIA | |
| | SAPGKCQDSAALTSTAFSGLDFGLLSGYLHKQALVTATHPTCTLLFP | |
| | SCHAFFPLPLTPTLYKMHKGWKNYCSQKSLNEASMDEYLGSLGLFR | |
| | KLTAKDASCLFRAISEQLFCSQVHHLEIRKACVSYMRENQQTFESYV | |
| | EGSFEKYLERLGDPKESAGQLEIRALSLIYNRDFILYRFPGKPPTYVT | |
| | DNGYEDKILLCYSSSGHYDSVYSKQFQSSAAVCQAVLYEILYKDVF | |
| | VVDEEELKTAIKLFRSGSKKNRNNAVTGSEDAHTDYKSSNQNRME | |
| | EWGACYNAENIPEGYNKGTEETKSPENPSKMPFPYKVLKALDPEIY | |
| | RNVEFDVWLDSRKELQKSDYMEYAGRQYYLGDKCQVCLESEGRY | |
| | YNAHIQEVGNENNSVTVFIEELAEKHVVPLANLKPVTQVMSVPAW | |
| | NAMPSRKGRGYQKMPGGYVPEIVISEMDIKQQKKMFKKIRGKEVY | |
| | M | |
| | TMAYGKGDPLLPPRLQHSMHYGHDPPMHYSQTAGNVMSNEHFHP | |
| | QHPSPRQGRGYGMPRNSSRFINRHNMPGPKVDFYPGPGKRCCQSYD | |
| | NFSYRSRSFRRSHRQMSCVNKESQYGFTPGNGQMPRGLEETITFYE | |
| | VEEGDETAYPTLPNHGGPSTMVPATSGYCVGRRGHSSGKQTLNLEE | |
| | GNGQSENGRYHEEYLYRAEPDYETSGVYSTTASTANLSLQDRKSCS | |
| | MSPQDTVTSYNYPQKMMGNIAAVAASCANNVPAPVLSNGAAANQ | |
| | AISTTSVSSQNAIQPLFVSPPTHGRPVIASPSYPCHSAIPHAGASLPPPP | |
| | PPPPPPPPPPPPPPPPPPPPPPPALDVGETSNLQPPPPLPPPPYSCDPSGS | |
| | DLPQDTKVLQYYFNLGLQCYYHSYWHSMVYVPQMQQQLHVENYP | |
| | VYTEPPLVDQTVPQCYSEVRREDGIQAEASANDTFPNADSSSVPHG | |
| | AVYYPVMSDPYGQPPLPGFDSCLPVVPDYSCVPPWHPVGTAYGGSS | |
| | QIHGAINPGPIGCIAPSPPASHYVPQGM | |
| 203 | MGSLFRSETMCLAQLFLQSGTAYECLSALGEKGLVQFRDLNQNVSS | ATP6V0A2 |
| | FQRKFVGEVKRCEELERILVYLVQEINRADIPLPEGEASPPAPPLKQV | |
| | LEMQEQLQKLEVELREVTKNKEKLRKNLLELIEYTHMLRVTKTFVK | |
| | RNVEFEPTYEEFPSLESDSLLDYSCMQRLGAKLGFVSGLINQGKVEA | |
| | FEKMLWRVCKGYTIVSYAELDESLEDPETGEVIKWYVFLISFWGEQI | |
| | GHKVKKICDCYHCHVYPYPNTAEERREIQEGLNTRIQDLYTVLHKT | |
| | EDYLRQVLCKAAESVYSRVIQVKKMKAIYHMLNMCSFDVTNKCLI | |
| | AEVWCPEADLQDLRRALEEGSRESGATIPSFMNIIPTKETPPTRIRTN | |
| | KFTEGFQNIVDAYGVGSYREVNPALFTIITFPFLFAVMFGDFGHGFV | |
| | MFLFALLLVLNENHPRLNQSQEIMRMFFNGRYILLLMGLFSVYTGLI | |
| | YNDCFSKSVNLFGSGWNVSAMYSSSHPPAEHKKMVLWNDSVVRH | |
| | NSILQLDPSIPGVFRGPYPLGIDPIWNLATNRLTFLNSFKMKMSVILGI | |
| | IHMTFGVILGIFNHLHFRKKFNIYLVSIPELLFMLCIFGYLIFMIFYKW | |
| | LVFSAETSRVAPSILIEFINMFLFPASKTSGLYTGQEYVQRVLLVVTA | |
| | LSVPVLFLGKPLFLLWLHNGRSCFGVNRSGYTLIRKDSEEEVSLLGS | |
| | QDIEEGNHQVEDGCREMACEEFNFGEILMTQVIHSIEYCLGCISNTA | |
| | SYLRLWALSLAHAQLSDVLWAMLMRVGLRVDTTYGVLLLLPVIAL | |
| | FAVLTIFILLIMEGLSAFLHAIRLHWVEFQNKFYVGAGTKFVPF | |
| | SFSLLSSKFNNDDSVA | |
| 204 | MRPPACWWLLAPPALLALLTCSLAFGLASEDTKKEVKQSQDLEKS | B3GLCT |
| | GISRKNDIDLKGIVFVIQSQSNSFHAKRAEQLKKSILKQAADLTQELP | |
| | SVLLLHQLAKQEGAWTILPLLPHFSVTYSRNSSWIFFCEEETRIQIPK | |
| | LLETLRRYDPSKEWFLGKALHDEEATIIHHYAFSENPTVFKYPDFAA | |
| | GWALSIPLVNKLTKRLKSESLKSDFTIDLKHEIALYIWDKGGGPPLTP | |
| | VPEF | |
| | CTNDVDFYCATTFHSFLPLCRKPVKKKDIFVAVKTCKKFHGDRIPIV | |
| | KQTWESQASLIEYYSDYTENSIPTVDLGIPNTDRGHCGKTFAILERFL | |
| | NRSQDKTAWLVIVDDDTLISISRLQHLLSCYDSGEPVFLGERYGYGL | |
| | GTGGYSYITGGGGMVFSREAVRRLLASKCRCYSNDAPDDMVLGMC | |
| | FSGLGIPVTHSPLFHQARPVDYPKDYLSHQVPISFHKHWNIDPVKVY | |
| | FTWLAPSDEDKARQETQKGFREEL | |
| 205 | MFPRPLTPLAAPNGAEPLGRALRRAPLGRARAGLGGPPLLLPSMLM | CHST14 |
| | FAVIVASSGLLLMIERGILAEMKPLPLHPPGREGTAWRGKAPKPGGL | |
| | SLRAGDADLQVRQDVRNRTLRAVCGQPGMPRDPWDLPVGQRRTL | |
| | LRHILVSDRYRFLYCYVPKVACSNWKRVMKVLAGVLDSVDVRLK | |
| | MDHRSDLVFLADLRPEEIRYRLQHYFKFLFVREPLERLLSAYRNKFG | |
| | EIREYQQRYGAEIVRRYRAGAGPSPAGDDVTFPEFLRYLVDEDPER | |
| | MNEHWMPVYHLCQPCAVHYDFVGSYERLEADANQVLEWVRAPPH | |
| | VRFPARQAWYRPASPESLHYHLCSAPRALLQDVLPKYILDFSLFAYP | |
| | LPNVTKEACQQ | |
| 206 | MATAATSPALKRLDLRDPAALFETHGAEEIRGLERQVRAEIEHKKE | COG1 |
| | ELRQMVGERYRDLIEAADTIGQMRRCAVGLVDAVKATDQYCARLR | |
| | QAGSAAPRPPRAQQPQQPSQEKFYSMAAQIKLLLEIPEKIWSSMEAS | |
| | QCLHATQLYLLCCHLHSLLQLDSSSSRYSPVLSRFPILIRQVAAASHF | |
| | RSTILHESKMLLKCQGVSDQAVAEALCSIMLLEESSPRQALTDFLLA | |
| | RKATIQKLLNQPHHGAGIKAQICSLVELLATTLKQAHALFYTLPEGL | |
| | LPDPALPCGLLFSTLETITGQHPAGKGTGVLQEEMKLCSWFKHLPAS | |
| | IVEFQPTLRTLAHPISQEYLKDTLQKWIHMCNEDIKNGITNLLMYVK | |
| | SMKGLAGIRDAMWELLTNESTNHSWDVLCRRLLEKPLLFWEDMM | |
| | QQLFLDRLQTLTKEGFDSISSSSKELLVSALQELESSTSNSPSNKHIHF | |
| | EYNMSLFLWSESPNDLPSDAAWVSVANRGQFASSGLSMKAQAISPC | |
| | VQNFCSALDSKLKVKLDDLLAYLPSDD | |
| | SSLPKDVSPTQAKSSAFDRYADAGTVQEMLRTQSVACIKHIVDCIRA | |
| | ELQSIEEGVQGQQDALNSAKLHSVLFMARLCQSLGELCPHLKQCIL | |
| | GKSESSEKPAREFRALRKQGKVKTQEIIPTQAKWQEVKEVLLQQSV | |
| | MGYQVWSSAVVKVLIHGFTQSLLLDDAGSVLATATSWDELEIQEEA | |
| | ESGSSVTSKIRLPAQPSWYVQSFLFSLCQEINRVGGHALPKVTLQEM | |
| | LKSCMVQVVAAYEKLSEEKQIKKEGAFPVTQNRALQLLYDLRYLNI | |
| | VLTAKGDEVKSGRSKPDSRIEK | |
| | VTDHLEALIDPFDLDVFTPHLNSNLHRLVQRTSVLFGLVTGTENQLA | |
| | PRSSTFNSQEPHNILPLASSQIRFGLLPLSMTSTRKAKSTRNIETKAQV | |
| | VPPARSTAGDPTVPGSLFRQLVSEEDNTSAPSLFKLGWLSSMTK | |
| 207 | MEKSRMNLPKGPDTLCFDKDEFMKEDFDVDHFVSDCRKRVQLEEL | COG2 |
| | RDDLELYYKLLKTAMVELINKDYADFVNLSTNLVGMDKALNQLSV | |
| | PLGQLREEVLSLRSSVSEGIRAVDERMSKQEDIRKKKMCVLRLIQVI | |
| | RSVEKIEKILNSQSSKETSALEASSPLLTGQILERIATEFNQLQFHAVQ | |
| | SKGMPLLDKVRPRIAGITAMLQQSLEGLLLEGLQTSDVDIIRHCLRT | |
| | YATIDKTRDAEALVGQVLVKPYIDEVIIEQFVESHPNGLQVMYNKLL | |
| | EFVPHHCRLLREVTGGAISSEKGNTVPGYDFLVNSVWPQIVQGLEE | |
| | KLPSLFNPGNPDAFHEKYTISMDFVRRLERQCGSQASVKRLRAHPA | |
| | YHSFNKKWNLPVYFQIRFREIAGSLEAALTDVLEDAPAESPYCLLAS | |
| | HRTWSSLRRCWSDEMFLPLLVHRLWRLTLQILARYSVFVNELSLRPI | |
| | SNESPKEIKKPLVTGSKEPSITQGNTEDQGSGPSETKPVVSISRTQLV | |
| | YVVADLDKLQEQLPELLEIIKPKLEMIGFKNFSSISAALEDSQSSFSA | |
| | CVPSLSSKIIQDLSDSCFGFLKSALEVPRLYRRTNKEVPTTASSYVDS | |
| | ALKPLFQLQSGHKDKLKQAIIQQWLEGTLSESTHKYYETVSDVLNS | |
| | VKKMEESLKRLKQARKTTPANPVGPSGGMSDDDKIRLQLALDVEY | |
| | LGEQIQKLGLQASDIKSFSALAELVAAAKDQATAEQP | |
| 208 | MADLDSPPKLSGVQQPSEGVGGGRCSEISAELIRSLTELQELEAVYE | COG4 |
| | RLCGEEKVVERELDALLEQQNTIESKMVTLHRMGPNLQLIEGDAKQ | |
| | LAGMITFTCNLAENVSSKVRQLDLAKNRLYQAIQRADDILDLKFCM | |
| | DGVQTALRSEDYEQAAAHTHRYLCLDKSVIELSRQGKEGSMIDANL | |
| | KLLQEAEQRLKAIVAEKFAIATKEGDLPQVERFFKIFPLLGLHEEGLR | |
| | KFSEYLCKQVASKAEENLLMVLGTDMSDRRAAVIFADTLTLLFEGI | |
| | ARIVETHQPIVETYYGPGRLYTLIKYLQVECDRQVEKVVDKFIKQRD | |
| | YHQQFRHVQNNLMRNSTTEKIEPRELDPILTEVTLMNARSELYLRFL | |
| | KKRISSDFEVGDSMASEEVKQEHQKCLDKLLNNCLLSCTMQELIGL | |
| | YVTMEEYFMRETVNKAVALDTYEKGQLTSSMVDDVFYIVKKCIGR | |
| | ALSSSSIDCLCAMINLATTELESDFRDVLCNKLRMGFPATTFQDIQR | |
| | GVTSAVNIMHSSLQQGKFDTKGIESTDEAKMSFLVTLNNVEVCSENI | |
| | STLKKTLESDCTKLFSQGIGGEQAQAKFDSCLSDLAAVSNKFRDLLQ | |
| | EGLTELNSTAIKPQVQPWINSFFSVSHNIEEEEFNDYEANDPWVQQFI | |
| | LNLEQQMAEFKASLSPVIYDSLTGLMTSLVAVELEKVVLKSTFNRL | |
| | GGLQFDKELRSLIAYLTTVTTWTIRDKFARLSQMATILNLERVTEILD | |
| | YWGPNSGPLTWRLTPAEVRQVLALRIDFRSEDIKRLRL | |
| 209 | MGWVGGRRRDSASPPGRSRSAADDINPAPANMEGGGGSVAVAGL | COG5 |
| | GARGSGAAAATVRELLQDGCYSDFLNEDFDVKTYTSQSIHQAVIAE | |
| | QLAKLAQGISQLDRELHLQVVARHEDLLAQATGIESLEGVLQMMQ | |
| | TRIGALQGAVDRIKAKIVEPYNKIVARTAQLARLQVACDLLRRIIRIL | |
| | NLSKRLQGQLQGGSREITKAAQSLNELDYLSQGIDLSGIEVIENDLLF | |
| | IARARLEVENQAKRLLEQGLETQNPTQVGTALQVFYNLGTLKDTITS | |
| | VVDGYCATLEENINSALDIKVLTQPSQSAVRGGPGRSTMPTPGNTA | |
| | ALRASFWTNMEKLMDHIYAVCGQVQHLQKVLAKKRDPVSHICFIE | |
| | EIVKDGQPEIFYTFWNSVTQALSSQFHMATNSSMFLKQAFEGEYPK | |
| | LLRLYNDLWKRLQQYSQHIQGNFNASGTTDLYVDLQHMEDDAQDI | |
| | FIPKKPDYDPEKALKDSLQPYEAAYLSKSLSRLFDPINLVFPPGGRNP | |
| | PSSDELDGIIKTIASELNVAAVDTNLTLAVSKNVAKTIQLYSVKSEQL | |
| | LSTQGDASQVIGPLTEGQRRNVAVVNSLYKLHQSVTKAIHALMENA | |
| | VQPLLTSVGDAIEAIIITMHQEDFSGSLSSSGKPDVPCSLYMKELQGF | |
| | IARVMSDYFKHFECLDFVFDNTEAIAQRAVELFIRHASLIRPLGEGG | |
| | KMRLAADFAQMELAVGPFCRRVSDLGKSYRMLRSFRPLLFQASEH | |
| | VASSPALGDVIPFSIIIQFLFTRAPAELKSPFQRAEWSHTRFSQWLDD | |
| | HPSEKDRLLLIRGALEAYVQSVRSREGKEFAPVYPIMVQLLQKAMS | |
| | ALQ | |
| 210 | MAEGSGEVVAVSATGAANGLNNGAGGTSATTCNPLSRKLHKILET | COG6 |
| | RLDNDKEMLEALKALSTFFVENSLRTRRNLRGDIERKSLAINEEFVSI | |
| | FKEVKEELESISEDVQAMSNCCQDMTSRLQAAKEQTQDLIVKTTKL | |
| | QSESQKLEIRAQVADAFLSKFQLTSDEMSLLRGTREGPITEDFFKAL | |
| | GRVKQIHNDVKVLLRTNQQTAGLEIMEQMALLQETAYERLYRWAQ | |
| | SECRTLTQESCDVSPVLTQAMEALQDRPVLYKYTLDEFGTARRSTV | |
| | VRGFIDALTRGGPGGTPRPIEMHSHDPLRYVGDMLAWLHQATASE | |
| | KEHLEALLKHVTTQGVEENIQEVVGHITEGVCRPLKVRIEQVIVAEP | |
| | GAVLLYKISNLLKFYHHTISGIVGNSATALLTTIEEMHLLSKKIFFNS | |
| | LSLHASKLMDKVELPPPDLGPSSALNQTLMLLREVLASHDSSVVPL | |
| | DARQADFVQVLSCVLDPLLQMCTVSASNLGTADMATFMVNSLYM | |
| | MKTTLALFEFTDRRLEMLQFQIEAHLDTLINEQASYVLTRVGLSYIY | |
| | NTVQQHKPEQGSLANMPNLDSVTLKAAMVQFDRYLSAPDNLLIPQ | |
| | LNFLLSATVKEQIVKQSTELVCRAYGEVYAAVMNPINEYKDPENIL | |
| | HRSPQQVQTLLS | |
| 211 | MDFSKFLADDFDVKEWINAAFRAGSKEAASGKADGHAATLVMKL | COG7 |
| | QLFIQEVNHAVEETSHQALQNMPKVLRDVEALKQEASFLKEQMILV | |
| | KEDIKKFEQDTSQSMQVLVEIDQVKSRMQLAAESLQEADKWSTLSA | |
| | DIEETFKTQDIAVISAKLTGMQNSLMMLVDTPDYSEKCVHLEALKN | |
| | RLEALASPQIVAAFTSQAVDQSKVFVKVFTEIDRMPQLLAYYYKCH | |
| | KVQLLAAWQELCQSDLSLDRQLTGLYDALLGAWHTQIQWATQVF | |
| | QKPHEVVMVLLIQTLGALMPSLPSCLSNGVERAGPEQELTRLLEFY | |
| | DATAHFAKGLEMALLPHLHEHNLVKVTELVDAVYDPYKPYQLKY | |
| | GDMEESNLLIQMSAVPLEHGEVIDCVQELSHSVNKLFGLASAAVDR | |
| | CVRFTNGLGTCGLLSALKSLFAKYVSDFTSTLQSIRKKCKLDHIPPNS | |
| | LFQEDWTAFQNSIRIIATCGELLRHCGDFEQQLANRILSTAGKYLSDS | |
| | CSPRSLAGFQESILTDKKNSAKNPWQEYNYLQKDNPAEYASLMEIL | |
| | YTLKEKGSSNHNLLAAPRAALTRLNQQAHQLAFDSVFLRIKQQLLLI | |
| | SKMDSWNTAGIGETLTDELPAFSLTPLEYISNIGQYIMSLPLNLEPFV | |
| | TQEDSALELALHAGKLPFPPEQGDELPELDNMADNWLGSIARATM | |
| | QTYCDAILQIPELSPHSAKQLATDIDYLINVMDALGLQPSRTLQHIVT | |
| | LLKTRPEDYRQVSKGLPRRLATTVATMRSVNY | |
| 212 | MATAATIPSVATATAAALGEVEDEGLLASLFRDRFPEAQWRERPDV | COG8 |
| | GRYLRELSGSGLERLRREPERLAEERAQLLQQTRDLAFANYKTFIRG | |
| | AECTERIHRLFGDVEASLGRLLDRLPSFQQSCRNFVKEAEEISSNRR | |
| | MNSLTLNRHTEILEILEIPQLMDTCVRNSYYEEALELAAYVRRLERK | |
| | YSSIPVIQGIVNEVRQSMQLMLSQLIQQLRTNIQLPACLRVIGYLRRM | |
| | DVFTEAELRVKFLQARDAWLRSILTAIPNDDPYFHITKTIEASRVHLF | |
| | DIITQYRAIFSDEDPLLPPAMGEHTVNESAIFHGWVLQKVSQFLQVL | |
| | ETDLYRGIGGHLDSLLGQCMYFGLSFSRVGADFRGQLAPVFQRVAI | |
| | STFQKAIQETVEKFQEEMNSYMLISAPAILGTSNMPAAVPATQPGTL | |
| | QPPMVLLDFPPLACFLNNILVAFNDLRLCCPVALAQDVTGALEDAL | |
| | AKVTKIILAFHRAEEAAFSSGEQELFVQFCTVFLEDLVPYLNRCLQV | |
| | LFPPAQIAQTLGIPPTQLSKYGNLGHVNIGAIQEPLAFILPKRETLFTL | |
| | DDQALGPELTAPAPEPPAEEPRLEPAGPACPEGGRAETQAEPPSVGP | |
| 213 | DRLLQQGSAVFQFRMSANSGLLPASMVMPLLGLVMKERCQTAGNP | DOLK |
| | FFERFGIVVAATGMAVALFSSVLALGITRPVPTNTCVILGLAGGVIIY | |
| | IMKHSLSVGEVIEVLEVLLIFVYLNMILLYLLPRCFTPGEALLVLGGI | |
| | SFVLNQLIKRSLTLVESQGDPVDFFLLVVVVGMVLMGIFFSTLFVFM | |
| | DSGTWASSIFFHLMTCVLSLGVVLPWLHRLIRRNPLLWLLQFLFQTD | |
| | TRIYLLAYWSLLATLACLVVLYQNAKRSSSESKKHQAPTIARKYFH | |
| | LIVVATYIPGIIFDRPLLYVAATVCLAVFIFLEYVRYFRIKPLGHTLRS | |
| | FLSLFLDERDSGPLILTHIYLLLGMSLPIWLIPRPCTQKGSLGGARAL | |
| | VPYAGVLAVGVGDTVASIFGSTMGEIRWPGTKKTFEGTMTSIFAQII | |
| | SVALILIFDSGVDLNYSYAWILGSISTVSLLEAYTTQIDNLLLPLYLLI | |
| | LLMA | |
| 214 | MSWIKEGELSLWERFCANIIKAGPMPKHIAFIMDGNRRYAKKCQVE | DHDDS |
| | RQEGHSQGFNKLAETLRWCLNLGILEVTVYAFSIENFKRSKSEVDGL | |
| | MDLARQKFSRLMEEKEKLQKHGVCIRVLGDLHLLPLDLQELIAQAV | |
| | QATKNYNKCFLNVCFAYTSRHEISNAVREMAWGVEQGLLDPSDISE | |
| | SLLDKCLYTNRSPHPDILIRTSGEVRLSDFLLWQTSHSCLVFQPVLW | |
| | PEYTFWNLFEAILQFQMNHSVLQKARDMYAEERKRQQLERDQATV | |
| | TEQLLREGLQASGDAQLRRTRLHKLSARREERVQGFLQALELKRAD | |
| | WLARLGTASA | |
| 215 | MWAFSELPMPLLINLIVSLLGFVATVTLIPAFRGHFIAARLCGQDLN | DPAGT1 |
| | KTSRQQIPESQGVISGAVFLIILFCFIPFPFLNCFVKEQCKAFPHHEFV | |
| | ALIGALLAICCMIFLGFADDVLNLRWRHKLLLPTAASLPLLMVYFTN | |
| | FGNTTIVVPKPFRPILGLHLDLGILYYVYMGLLAVFCTNAINILAGIN | |
| | GLEAGQSLVISASIIVFNLVELEGDCRDDHVFSLYFMIPFFFTTLGLL | |
| | YHNWYPSRVFVGDTFCYFAGMTFAVVGILGHFSKTMLLFFMPQVF | |
| | NFLYSLPQLLHIIPCPRHRIPRLNIKTGKLEMSYSKFKTKSLSFLGTFIL | |
| | KVAESLQLVTVHQSETEDGEFTECNNMTLINLLLKVLGPIHERNLTL | |
| | LLLLLQILGSAITFSIRYQLVRLFYDV | |
| 216 | MASLEVSRSPRRSRRELEVRSPRQNKYSVLLPTYNERENLPLIVWLL | DPM1 |
| | VKSFSESGINYEIIIIDDGSPDGTRDVAEQLEKIYGSDRILLRPREKKL | |
| | GLGTAYIHGMKHATGNYIIIMDADLSHHPKFIPEFIRKQKEGNFDIVS | |
| | GTRYKGNGGVYGWDLKRKIISRGANFLTQILLRPGASDLTGSFRLY | |
| | RKEVLEKLIEKCVSKGYVFQMEMIVRARQLNYTIGEVPISFVDRVY | |
| | GESK | |
| | LGGNEIVSFLKGLLTLFATT | |
| 217 | MATGTDQVVGLGLVAVSLIIFTYYTAWVILLPFIDSQHVIHKYFLPR | DPM2 |
| | AYAVAIPLAAGLLLLLFVGLFISYVMLKTKRVTKKAQ | |
| 218 | MTKLAQWLWGLAILGSTWVALTTGALGLELPLSCQEVLWPLPAYL | DPM3 |
| | LVSAGCYALGTVGYRVATFHDCEDAARELQSQIQEARADLARRGL | |
| | RF | |
| 219 | MESTLGAGIVIAEALQNQLAWLENVWLWITFLGDPKILFLFYFPAAY | G6PC3 |
| | YASRRVGIAVLWISLITEWLNLIFKWFLFGDRPFWWVHESGYYSQA | |
| | PAQVHQFPSSCETGPGSPSGHCMITGAALWPIMTALSSQVATRARSR | |
| | WVRVMPSLAYCTFLLAVGLSRIFILAHFPHQVLAGLITGAVLGWLM | |
| | TPRVPMERELSFYGLTALALMLGTSLIYWTLFTLGLDLSWSISLAFK | |
| | WCERPEWIHVDSRPFASLSRDSGAALGLGIALHSPCYAQVRRAQLG | |
| | NGQKIACLVLAMGLLGPLDWLGHPPQISLFYIFNFLKYTLWPCLVL | |
| | ALVPWAVHMFSAQEAPPIHSS | |
| 220 | MCGIFAYLNYHVPRTRREILETLIKGLQRLEYRGYDSAGVGFDGGN | GFPT1 |
| | DKDWEANACKIQLIKKKGKVKALDEEVHKQQDMDLDIEFDVHLGI | |
| | AHTRWATHGEPSPVNSHPQRSDKNNEFIVIHNGIITNYKDLKKFLES | |
| | KGYDFESETDTETIAKLVKYMYDNRESQDTSFTTLVERVIQQLEGAF | |
| | ALVFKSVHFPGQAVGTRRGSPLLIGVRSEHKLSTDHIPILYRTARTQI | |
| | GSKFTRWGSQGERGKDKKGSCNLSRVDSTTCLFPVEEKAVEYYFAS | |
| | DASAVIEHTNRVIFLEDDDVAAVVDGRLSIHRIKRTAGDHPGRAVQ | |
| | TLQMELQQIMKGNFSSFMQKEIFEQPESVVNTMRGRVNFDDYTVNL | |
| | GGLKDHIKEIQRCRRLILIACGTSYHAGVATRQVLEELTELPVMVEL | |
| | ASDFLDRNTPVFRDDVCFFLSQSGETADTLMGLRYCKERGALTVGI | |
| | TNTVGSSISRETDCGVHINAGPEIGVASTKAYTSQFVSLVMFALMM | |
| | CDDRISMQERRKEIMLGLKRLPDLIKEVLSMDDEIQKLATELYHQKS | |
| | VLIMGRGYHYATCLEGALKIKEITYMHSEGILAGELKHGPLALVDK | |
| | LMPVIMIIMRDHTYAKCQNALQQVVARQGRPVVICDKEDTETIKNT | |
| | KRTIKVPHSVDCLQGILSVIPLQLLAFHLAVLRGYDVDFPRNLAKSV | |
| | TVE | |
| 221 | MLKAVILIGGPQKGTRFRPLSFEVPKPLFPVAGVPMIQHHIEACAQV | GMPPA |
| | PGMQEILLIGFYQPDEPLTQFLEAAQQEFNLPVRYLQEFAPLGTGGG | |
| | LYHFRDQILAGSPEAFFVLNADVCSDFPLSAMLEAHRRQRHPFLLLG | |
| | TTANRTQSLNYGCIVENPQTHEVLHYVEKPSTFISDIINCGIYLFSPEA | |
| | LKPLRDVFQRNQQDGQLEDSPGLWPGAGTIRLEQDVFSALAGQGQI | |
| | YVHL | |
| | TDGIWSQIKSAGSALYASRLYLSRYQDTHPERLAKHTPGGPWIRGN | |
| | VYIHPTAKVAPSAVLGPNVSIGKGVTVGEGVRLRESIVLHGATLQEH | |
| | TCVLHSIVGWGSTVGRWARVEGTPSDPNPNDPRARMDSESLFKDG | |
| | KLLPAITILGCRVRIPAEVLILNSIVLPHKELSRSFTNQIIL | |
| 222 | MKALILVGGYGTRLRPLTLSTPKPLVDFCNKPILLHQVEALAAAGV | GMPPB |
| | DHVILAVSYMSQVLEKEMKAQEQRLGIRISMSHEEEPLGTAGPLAL | |
| | ARDLLSETADPFFVLNSDVICDFPFQAMVQFHRHHGQEGSILVTKVE | |
| | EPSKYGVVVCEADTGRIHRFVEKPQVFVSNKINAGMYILSPAVLQRI | |
| | QLQPTSIEKEVFPIMAKEGQLYAMELQGFWMDIGQPKDFLTGMCLF | |
| | LQSLRQKQPERLCSGPGIVGNVLVDPSARIGQNCSIGPNVSLGPGVV | |
| | VEDGVCIRRCTVLRDARIRSHSWLESCIVGWRCRVGQWVRMENVT | |
| | VLGEDVIVNDELYLNGASVLPHKSIGESVPEPRIIM | |
| 223 | MAARWRFWCVSVTMVVALLIVCDVPSASAQRKKEMVLSEKVSQL | MAGT1 |
| | MEWTNKRPVIRMNGDKFRRLVKAPPRNYSVIVMFTALQLHRQCVV | |
| | CKQADEEFQILANSWRYSSAFTNRIFFAMVDFDEGSDVFQMLNMNS | |
| | APTFINFPAKGKPKRGDTYELQVRGFSAEQIARWIADRTDVNIRVIR | |
| | PPNYAGPLMLGLLLAVIGGLVYLRRSNMEFLFNKTGWAFAALCFVL | |
| | AMTSGQMWNHIRGPPYAHKNPHTGHVNYIHGSSQAQFVAETHIVL | |
| | LFNGGVTLGMVLLCEAATSDMDIGKRKIMCVAGIGLVVLFFSWML | |
| | SIFRSKYHGYPYSFLMS | |
| 224 | MAACEGRRSGALGSSQSDFLTPPVGGAPWAVATTVVMYPPPPPPPH | MAN1B1 |
| | RDFISVTLSFGENYDNSKSWRRRSCWRKWKQLSRLQRNMILFLLAF | |
| | LLFCGLLFYINLADHWKALAFRLEEEQKMRPEIAGLKPANPPVLPAP | |
| | QKADTDPENLPEISSQKTQRHIQRGPPHLQIRPPSQDLKDGTQEEAT | |
| | KRQEAPVDPRPEGDPQRTVISWRGAVIEPEQGTELPSRRAEVPTKPP | |
| | LPPARTQGTPVHLNYRQKGVIDVFLHAWKGYRKFAWGHDELKPVS | |
| | RSFSEWFGLGLTLIDALDTMWILGLRKEFEEARKWVSKKLHFEKDV | |
| | DVNLFESTIRILGGLLSAYHLSGDSLFLRKAEDFGNRLMPAFRTPSKI | |
| | PYSDVNIGTGVAHPPRWTSDSTVAEVTSIQLEFRELSRLTGDKKFQE | |
| | AVEKVTQHIHGLSGKKDGLVPMFINTHSGLFTHLGVFTLGARADSY | |
| | YEYLLKQWIQGGKQETQLLEDYVEAIEGVRTHLLRHSEPSKLTFVG | |
| | ELAHGRFSAKMDHLVCFLPGTLALGVYHGLPASHMELAQELMETC | |
| | YQMNRQMETGLSPEIVHFNLYPQPGRRDVEVKPADRHNLLRPETVE | |
| | SLFYLYRVTGDRKYQDWGWEILQSFSRFTRVPSGGYSSINNVQDPQ | |
| | KPEPRDKMESFFLGETLKYLFLLFSDDPNLLSLDAYVFNTEAHPLPI | |
| | WTPA | |
| 225 | MRFRIYKRKVLILTLVVAACGFVLWSSNGRQRKNEALAPPLLDAEP | MGAT2 |
| | ARGAGGRGGDHPSVAVGIRRVSNVSAASLVPAVPQPEADNLTLRY | |
| | RSLVYQLNFDQTLRNVDKAGTWAPRELVLVVQVHNRPEYLRLLLD | |
| | SLRKAQGIDNVLVIFSHDFWSTEINQLIAGVNFCPVLQVFFPFSIQLY | |
| | PNEFPGSDPRDCPRDLPKNAALKLGCINAEYPDSFGHYREAKFSQTK | |
| | HHWWWKLHFVWERVKILRDYAGLILFLEEDHYLAPDFYHVFKKM | |
| | WKLKQQECPECDVLSLGTYSASRSF | |
| | YGMADKVDVKTWKSTEHNMGLALTRNAYQKLIECTDTFCTYDDY | |
| | NWDWTLQYLTVSCLPKFWKVLVPQIPRIFHAGDCGMHHKKTCRPS | |
| | TQSAQIESLLNNNKQYMFPETLTISEKFTVVAISPPRKNGGWGDIRD | |
| | HELCKSYRRLQ | |
| 226 | MARGERRRRAVPAEGVRTAERAARGGPGRRDGRGGGPRSTAGGV | MOGS |
| | ALAVVVLSLALGMSGRWVLAWYRARRAVTLHSAPPVLPADSSSPA | |
| | VAPDLFWGTYRPHVYFGMKTRSPKPLLTGLMWAQQGTTPGTPKLR | |
| | HTCEQGDGVGPYGWEFHDGLSFGRQHIQDGALRLTTEFVKRPGGQ | |
| | HGGDWSWRVTVEPQDSGTSALPLVSLFFYVVTDGKEVLLPEVGAK | |
| | GQLKFISGHTSELGDFRFTLLPPTSPGDTAPKYGSYNVFWTSNPGLP | |
| | LLTEMVKSRLNSWFQHRPPGAPPERYLGLPGSLKWEDRGPSGQGQ | |
| | GQFLIQQVTLKIPISIEFVFESGSAQAGGNQALPRLAGSLLTQALESH | |
| | AEGFRERFEKTFQLKEKGLSSGEQVLGQAALSGLLGGIGYFYGQGL | |
| | VLPDIGVEGSEQKVDPALFPPVPLFTAVPSRSFFPRGFLWDEGFHQL | |
| | VVQRWDPSLTREALGHWLGLLNADGWIGREQILGDEARARVPPEF | |
| | LVQRAVHANPPTLLLPVAHMLEVGDPDDLAFLRKALPRLHAWFSW | |
| | LHQSQAGPLPLSYRWRGRDPALPTLLNPKTLPSGLDDYPRASHPSVT | |
| | ERHLDLRCWVALGARVLTRLAEHLGEAEVAAELGPLAASLEAAES | |
| | LDELHWAPELGVFADFGNHTKAVQLKPRPPQGLVRVVGRPQPQLQ | |
| | YVDALGYVSLFPLLLRLLDPTSSRLGPLLDILADSRHLWSPFGLRSL | |
| | AASSSFYGQRNSEHDPPYWRGAVWLNVNYLALGALHHYGHLEGP | |
| | HQARAAKLHGELRANVVGNVWRQYQATGFLWEQYSDRDGRGMG | |
| | CRPFHGWTSLVLLAMAEDY | |
| 227 | MAAEADGPLKRLLVPILLPEKCYDQLFVQWDLLHVPCLKILLSKGL | MPDU1 |
| | GLGIVAGSLLVKLPQVFKILGAKSAEGLSLQSVMLELVALTGTMVY | |
| | SITNNFPFSSWGEALFLMLQTITICFLVMHYRGQTVKGVAFLACYGL | |
| | VLLVLLSPLTPLTVVTLLQASNVPAVVVGRLLQAATNYHNGHTGQL | |
| | SAITVFLLFGGSLARIFTSIQETGDPLMAGTFVVSSLCNGLIAAQLLF | |
| | YWNAKPPHKQKKAQ | |
| 228 | MAAPRVFPLSCAVQQYAWGKMGSNSEVARLLASSDPLAQIAEDKP | MPI |
| | YAELWMGTHPRGDAKILDNRISQKTLSQWIAENQDSLGSKVKDTFN | |
| | GNLPFLFKVLSVETPLSIQAHPNKELAEKLHLQAPQHYPDANHKPE | |
| | MAIALTPFQGLCGFRPVEEIVTFLKKVPEFQFLIGDEAATHLKQTMS | |
| | HDSQAVASSLQSCFSHLMKSEKKVVVEQLNLLVKRISQQAAAGNN | |
| | MEDIFGELLLQLHQQYPGDIGCFAIYFLNLLTLKPGEAMFLEANVPH | |
| | AYLKGDCVECMACSDNTVRAGLTP | |
| | KFIDVPTLCEMLSYTPSSSKDRLFLPTRSQEDPYLSIYDPPVPDFTIMK | |
| | TEVPGSVTEYKVLALDSASILLMVQGTVIASTPTTQTPIPLQRGGVLF | |
| | IGANESVSLKLTEPKDLLIFRACCLL | |
| 229 | MAAAALGSSSGSASPAVAELCQNTPETFLEASKLLLTYADNILRNPN | NGLY1 |
| | DEKYRSIRIGNTAFSTRLLPVRGAVECLFEMGFEEGETHLIFPKKASV | |
| | EQLQKIRDLIAIERSSRLDGSNKSHKVKSSQQPAASTQLPTTPSSNPS | |
| | GLNQHTRNRQGQSSDPPSASTVAADSAILEVLQSNIQHVLVYENPAL | |
| | QEKALACIPVQELKRKSQEKLSRARKLDKGINISDEDFLLLELLHWF | |
| | KEE | |
| | FFHWVNNVLCSKCGGQTRSRDRSLLPSDDELKWGAKEVEDHYCDA | |
| | CQFSNRFPRYNNPEKLLETRCGRCGEWANCFTLCCRAVGFEARYV | |
| | WDYTDHVWTEVYSPSQQRWLHCDACEDVCDKPLLYEIGWGKKLS | |
| | YVIAFSKDEVVDVTWRYSCKHEEVIARRTKVKEALLRDTINGLNKQ | |
| | RQLFLSENRRKELLQRIIVELVEFISPKTPKPGELGGRISGSVAWRVA | |
| | RGEMGLQRKETLFIPCENEKISKQLHLCYNIVKDRYVRVSNNNQTIS | |
| | GWENGVWKMESIFRKVETDWHMVYLARKEGSSFAYISWKFECGS | |
| | VGLKVDSISIRTSSQTFQTGTVEWKLRSDTAQVELTGDNSLHSYADF | |
| | SGATEVILEAELSRGDGDVAWQHTQLFRQSLNDHEENCLEIIIKFSDL | |
| 230 | MVKIVTVKTQAYQDQKPGTSGLRKRVKVFQSSANYAENFIQSIISTV | PGM1 |
| | EPAQRQEATLVVGGDGRFYMKEAIQLIARIAAANGIGRLVIGQNGIL | |
| | STPAVSCIIRKIKAIGGIILTASHNPGGPNGDFGIKFNISNGGPAPEAIT | |
| | DKIFQISKTIEEYAVCPDLKVDLGVLGKQQFDLENKFKPFTVEIVDS | |
| | VEAYATMLRSIFDFSALKELLSGPNRLKIRIDAMHGVVGPYVKKILC | |
| | EELGAPANSAVNCVPLEDFGGHHPDPNLTYAADLVETMKSGEHDF | |
| | GAAFDGDGDRNMILGKHGFFVNPSDSVAVIAANIFSIPYFQQTGVRG | |
| | FARSMPTSGALDRVASATKIALYETPTGWKFFGNLMDASKLSLCGE | |
| | ESFGTGSDHIREKDGLWAVLAWLSILATRKQSVEDILKDHWQKYGR | |
| | NFFTRYDYEEVEAEGANKMMKDLEALMFDRSFVGKQFSANDKVY | |
| | TVEKADNFEYSDPVDGSISRNQGLRLIFTDGSRIVFRLSGTGSAGATI | |
| | RLYIDSYEKDVAKINQDPQVMLAPLISIALKVSQLQERTGRTAPTVIT | |
| 231 | MDLGAITKYSALHAKPNGLILQYGTAGFRTKAEHLDHVMFRMGLL | PGM3 |
| | AVLRSKQTKSTIGVMVTASHNPEEDNGVKLVDPLGEMLAPSWEEH | |
| | ATCLANAEEQDMQRVLIDISEKEAVNLQQDAFVVIGRDTRPSSEKLS | |
| | QSVIDGVTVLGGQFHDYGLLTTPQLHYMVYCRNTGGRYGKATIEG | |
| | YYQKLSKAFVELTKQASCSGDEYRSLKVDCANGIGALKLREMEHY | |
| | FSQGLSVQLFNDGSKGKLNHLCGADFVKSHQKPPQGMEIKSNERCC | |
| | SFDGDADRIVYYYHDADGHFHLIDGDKIATLISSFLKELLVEIGESLN | |
| | IGVVQTAYANGSSTRYLEEVMKVPVYCTKTGVKHLHHKAQEFDIG | |
| | VYFEANGHGTALFSTAVEMKIKQSAEQLEDKKRKAAKMLENIIDLF | |
| | NQAAGDAISDMLVIEAILALKGLTVQQWDALYTDLPNRQLKVQVA | |
| | DRRVISTTDAERQAVTPPGLQEAINDLVKKYKLSRAFVRPSGTEDV | |
| | VRVYAEADSQESADHLAHEVSLAVFQLAGGIGERPQPGF | |
| 232 | MGSQEVLGHAARLASSGLLLQVLFRLITFVLNAFILRFLSKEIVGVV | RFT1 |
| | NVRLTLLYSTTLFLAREAFRRACLSGGTQRDWSQTLNLLWLTVPLG | |
| | VFWSLFLGWIWLQLLEVPDPNVVPHYATGVVLFGLSAVVELLGEPF | |
| | WVLAQAHMFVKLKVIAESLSVILKSVLTAFLVLWLPHWGLYIFSLA | |
| | QLFYTTVLVLCYVIYFTKLLGSPESTKLQTLPVSRITDLLPNITRNGA | |
| | FINWKEAKLTWSFFKQSFLKQILTEGERYVMTFLNVLNFGDQGVYD | |
| | IVNNLGSLVARLIFQPIEESFYIFFAKVLERGKDATLQKQEDVAVAA | |
| | AVLESLLKLALLAGLTITVFGFAYSQLALDIYGGTMLSSGSGPVLLR | |
| | SYCLYVLLLAINGVTECFTFAAMSKEEVDRYNFVMLALSSSFLVLS | |
| | YLLTRWCGSVGFILANCFNMGIRITQSLCFIHRYYRRSPHRPLAGLH | |
| | LSPVLLGTFALSGGVTAVSEVFLCCEQGWPARLAHIAVGAFCLGAT | |
| | LGTAFLTETKLIHFLRTQLGVPRRTDKMT | |
| 233 | MATYLEFIQQNEERDGVRFSWNVWPSSRLEATRMVVPLACLLTPLK | SEC23B |
| | ERPDLPPVQYEPVLCSRPTCKAVLNPLCQVDYRAKLWACNFCFQRN | |
| | QFPPAYGGISEVNQPAELMPQFSTIEYVIQRGAQSPLIFLYVVDTCLE | |
| | EDDLQALKESLQMSLSLLPPDALVGLITFGRMVQVHELSCEGISKSY | |
| | VFRGTKDLTAKQIQDMLGLTKPAMPMQQARPAQPQEHPFASSRFL | |
| | QPVHKIDMNLTDLLGELQRDPWPVTQGKRPLRSTGVALSIAVGLLE | |
| | GTFPNTGARIMLFTGGPPTQGPGMVVGDELKIPIRSWHDIEKDNARF | |
| | MKKATKHYEMLANRTAANGHCIDIYACALDQTGLLEMKCCANLT | |
| | GGYMVMGDSFNTSLFKQTFQRIFTKDFNGDFRMAFGATLDVKTSR | |
| | ELKIAGAIGPCVSLNVKGPCVSENELGVGGTSQWKICGLDPTSTLGI | |
| | YFEVVNQHNTPIPQGGRGAIQFVTHYQHSSTQRRIRVTTIARNWAD | |
| | VQSQLRHIEAAFDQEAAAVLMARLGVFRAESEEGPDVLRWLDRQLI | |
| | RLCQKFGQYNKEDPTSFRLSDSFSLYPQFMFHLRRSPFLQVFNNSPD | |
| | ESSYYRHHFARQDLTQSLIMIQPILYSYSFHGPPEPVLLDSSSILADRI | |
| | LLMDTFFQIVIYLGETIAQWRKAGYQDMPEYENFKHLLQAPLDDAQ | |
| | EILQARFPMPRYINTEHGGSQARFLLSKVNPSQTHNNLYAWGQETG | |
| | APILTDDVSLQVFMDHLKKLAVSSAC | |
| 234 | MAAPRDNVTLLFKLYCLAVMTLMAAVYTIALRYTRTSDKELYFST | SLC35A1 |
| | TAVCITEVIKLLLSVGILAKETGSLGRFKASLRENVLGSPKELLKLSV | |
| | PSLVYAVQNNMAFLALSNLDAAVYQVTYQLKIPCTALCTVLMLNR | |
| | TLSKLQWVSVFMLCAGVTLVQWKPAQATKVVVEQNPLLGFGAIAI | |
| | AVLCSGFAGVYFEKVLKSSDTSLWVRNIQMYLSGIIVTLAGVYLSD | |
| | GAEIKEKGFFYGYTYYVWFVIFLASVGGLYTSVVVKYTDNIMKGFS | |
| | AAAAIVLSTIASVMLFGLQITLTFALGTLLVCVSIYLYGLPRQDTTSI | |
| | QQGETASKERVIGV | |
| 235 | MAAVGAGGSTAAPGPGAVSAGALEPGTASAAHRRLKYISLAVLVV | SLC35A2 |
| | QNASLILSIRYARTLPGDRFFATTAVVMAEVLKGLTCLLLLFAQKRG | |
| | NVKHLVLFLHEAVLVQYVDTLKLAVPSLIYTLQNNLQYVAISNLPA | |
| | ATFQVTYQLKILTTALFSVLMLNRSLSRLQWASLLLLFTGVAIVQAQ | |
| | QAGGGGPRPLDQNPGAGLAAVVASCLSSGFAGVYFEKILKGSSGSV | |
| | WLRNLQLGLFGTALGLVGLWWAEGTAVATRGFFFGYTPAVWGVV | |
| | LNQAFGGLLVAVVVKYADNILKGFATSLSIVLSTVASIRLFGFHVDP | |
| | LFALGAGLVIGAVYLYSLPRGAAKAIASASASASGPCVHQQPPGQPP | |
| | PPQLSSHRGDLITEPFLPKLLTKVKGS | |
| 236 | MNRAPLKRSRILHMALTGASDPSAEAEANGEKPFLLRALQIALVVS | SLC35C1 |
| | LYWVTSISMVFLNKYLLDSPSLRLDTPIFVTFYQCLVTTLLCKGLSA | |
| | LAACCPGAVDFPSLRLDLRVARSVLPLSVVFIGMITFNNLCLKYVGV | |
| | AFYNVGRSLTTVFNVLLSYLLLKQTTSFYALLTCGIIIGGFWLGVDQ | |
| | EGAEGTLSWLGTVFGVLASLCVSLNAIYTTKVLPAVDGSIWRLTFY | |
| | NNVNACILFLPLLLLLGELQALRDFAQLGSAHFWGMMTLGGLFGFA | |
| | IGYVTGLQIKFTSPLTHNVSG | |
| | TAKACAQTVLAVLYYEETKSFLWWTSNMMVLGGSSAYTWVRGW | |
| | EMKKTPEEPSPKDSEKSAMGV | |
| 237 | MAAMASLGALALLLLSSLSRCSAEACLEPQITPSYYTTSDAVISTET | SSR4 |
| | VFIVEISLTCKNRVQNMALYADVGGKQFPVTRGQDVGRYQVSWSL | |
| | DHKSAHAGTYEVRFFDEESYSLLRKAQRNNEDISIIPPLFTVSVDHR | |
| | GTWNGPWVSTEVLAAAIGLVIYYLAFSAKSHIQA | |
| 238 | MAPWAEAEHSALNPLRAVWLTLTAAFLLTLLLQLLPPGLLPGCAIF | SRD5A3 |
| | QDLIRYGKTKCGEPSRPAACRAFDVPKRYFSHFYIISVLWNGFLLWC | |
| | LTQSLFLGAPFPSWLHGLLRILGAAQFQGGELALSAFLVLVFLWLHS | |
| | LRRLFECLYVSVFSNVMIHVVQYCFGLVYYVLVGLTVLSQVPMDG | |
| | RNAYITGKNLLMQARWFHILGMMMFIWSSAHQYKCHVILGNLRKN | |
| | KAGVVIHCNHRIPFGDWFEYVSSPNYLAELMIYVSMAVTFGFHNLT | |
| | WWLVVTNVFFNQALSAFLSHQFYKSKFVSYPKHRKAFLPFLF | |
| 239 | MAAAAPGNGRASAPRLLLLFLVPLLWAPAAVRAGPDEDLSHRNKE | TMEM165 |
| | PPAPAQQLQPQPVAVQGPEPARVEKIFTPAAPVHTNKEDPATQTNL | |
| | GFIHAFVAAISVIIVSELGDKTFFIAAIMAMRYNRLTVLAGAMLALG | |
| | LMTCLSVLFGYATTVIPRVYTYYVSTVLFAIFGIRMLREGLKMSPDE | |
| | GQEELEEVQAELKKKDEEFQRTKLLNGPGDVETGTSITVPQKKWLH | |
| | FISPIFVQALTLTFLAEWGDRSQLTTIVLAAREDPYGVAVGGTVGHC | |
| | LCTGLAVIGGRMIAQKISVRTVTIIGGIVFLAFAFSALFISPDSGF | |
| 240 | MSSWLGGLGSGLGQSLGQVGGSLASLTGQISNFTKDMLMEGTEEV | TRIP11 |
| | EAELPDSRTKEIEAIHAILRSENERLKKLCTDLEEKHEASEIQIKQQST | |
| | SYRNQLQQKEVEISHLKARQIALQDQLLKLQSAAQSVPSGAGVPAT | |
| | TASSSFAYGISHHPSAFHDDDMDFGDIISSQQEINRLSNEVSRLESEV | |
| | GHWRHIAQTSKAQGTDNSDQSEICKLQNIIKELKQNRSQEIDDHQHE | |
| | MSVLQNAHQQKLTEISRRHREELSDYEERIEELENLLQQGGSGVIET | |
| | DLSKIYEMQKTIQVLQIEKVESTKKMEQLEDKIKDINKKLSSAENDR | |
| | DILRREQEQLNVEKRQIMEECENLKLECSKLQPSAVKQSDTMTEKE | |
| | RILAQSASVEEVFRLQQALSDAENEIMRLSSLNQDNSLAEDNLKLK | |
| | MRIEVLEKEKSLLSQEKEELQMSLLKLNNEYEVIKSTATRDISLDSEL | |
| | HDLRLNLEAKEQELNQSISEKETLIAEIEELDRQNQEATKHMILIKDQ | |
| | LSKQQNEGDSIISKLKQDLNDEKKRVHQLEDDKMDITKELDVQKEK | |
| | LIQSEVALNDLHLTKQKLEDKVENLVDQLNKSQESNVSIQKENLEL | |
| | KEHIRQNEEELSRIRNELMQSLNQDSNSNFKDTLLKEREAEVRNLKQ | |
| | NLSELEQLNENLKKVAFDVKMENEKLVLACEDVRHQLEECLAGNN | |
| | QLSLEKNTIVETLKMEKGEIEAELCWAKKRLLEEANKYEKTIEELSN | |
| | ARNLNTSALQLEHEHLIKLNQKKDMEIAELKKNIEQMDTDHKETKD | |
| | VLSSSLEEQKQLTQLINKKEIFIEKLKERSSKLQEELDKYSQALRKNE | |
| | ILRQTIEEKDRSLGSMKEENNHLQEELERLREEQSRTAPVADPKTLD | |
| | SVTELASEVSQLNTIKEHLEEEIKHHQKIIEDQNQSKMQLLQSLQEQ | |
| | KKEMDEFRYQHEQMNATHTQLFLEKDEEIKSLQKTIEQIKTQLHEER | |
| | QDIQTDNSDIFQETKVQSLNIENGSEKHDLSKAETERLVKGIKERELE | |
| | IKLLNEKNISLTKQIDQLSKDEVGKLTQIIQQKDLEIQALHARISSTSH | |
| | TQDVVYLQQQLQAYAMEREKVFAVLNEKTRENSHLKTEYHKMMD | |
| | IVAAKEAALIKLQDENKKLSTRFESSGQDMFRETIQNLSRIIREKDIEI | |
| | DALSQKCQTLLAVLQTSSTGNEAGGVNSNQFEELLQERDKLKQQV | |
| | KKMEEWKQQVMTTVQNMQHESAQLQEELHQLQAQVLVDSDNNS | |
| | KLQVDYTGLIQSYEQNETKLKNFGQELAQVQHSIGQLCNTKDLLLG | |
| | KLDIISPQLSSASLLTPQSAECLRASKSEVLSESSELLQQELEELRKSL | |
| | QEKDATIRTLQENNHRLSDSIAATSELERKEHEQTDSEIKQLKEKQD | |
| | VLQKLLKEKDLLIKAKSDQLLSSNENFTNKVNENELLRQAVTNLKE | |
| | RILILEMDIGKLKGENEKIVETYRGKETEYQALQETNMKFSMMLRE | |
| | KEFECHSMKEKALAFEQLLKEKEQGKTGELNQLLNAVKSMQEKTV | |
| | VFQQERDQVMLALKQKQMENTALQNEVQRLRDKEFRSNQELERLR | |
| | NHLLESEDSYTREALAAEDREAKLRKKVTVLEEKLVSSSNAMENAS | |
| | HQASVQVESLQEQLNVVSKQRDETALQLSVSQEQVKQYALSLANL | |
| | QMVLEHFQQEEKAMYSAELEKQKQLIAEWKKNAENLEGKVISLQE | |
| | CLDEANAALDSASRLTEQLDVKEEQIEELKRQNELRQEMLDDVQK | |
| | KLMSLANSSEGKVDKVLMRNLFIGHFHTPKNQRHEVLRLMGSILGV | |
| | RREEMEQLFHDDQGGVTRWMTGWLGGGSKSVPNTPLRPNQQSVV | |
| | NSSFSELFVKFLETESHPSIPPPKLSVHDMKPLDSPGRRKRDTNAPES | |
| | FKDTAESRSGRRTDVNPFLAPRSAAVPLINPAGLGPGGPGHLLLKPIS | |
| | DVLPTFTPLPALPDNSAGVVLKDLLKQ | |
| 241 | MGARGAPSRRRQAGRRLRYLPTGSFPFLLLLLLLCIQLGGGQKKKE | TUSC3 |
| | NLLAEKVEQLMEWSSRRSIFRMNGDKFRKFIKAPPRNYSMIVMFTA | |
| | LQPQRQCSVCRQANEEYQILANSWRYSSAFCNKLFFSMVDYDEGT | |
| | DVFQQLNMNSAPTFMHFPPKGRPKRADTFDLQRIGFAAEQLAKWIA | |
| | DRTDVHIRVFRPPNYSGTIALALLVSLVGGLLYLRRNNLEFIYNKTG | |
| | WAMVSLCIVFAMTSGQMWNHIRGPPYAHKNPHNGQVSYIHGSSQA | |
| | QFVAESHIILVLNAAITMGMVLLNE | |
| | AATSKGDVGKRRIICLVGLGLVVFFFSFLLSIFRSKYHGYPYSDLDFE | |
| 242 | MVCVLVLAAAAGAVAVFLILRIWVVLRSMDVTPRESLSILVVAGSG | ALG14 |
| | GHTTEILRLLGSLSNAYSPRHYVIADTDEMSANKINSFELDRADRDP | |
| | SNMYTKYYIHRIPRSREVQQSWPSTVFTTLHSMWLSFPLIHRVKPDL | |
| | VLCNGPGTCVPICVSALLLGILGIKKVIIVYVESICRVETLSMSGKILF | |
| | HLSDYFIVQWPALKEKYPKSVYLGRIV | |
| 243 | MRLREPLLSGSAAMPGASLQRACRLLVAVCALHLGVTLVYYLAGR | B4GALT1 |
| | DLSRLPQLVGVSTPLQGGSNSAAAIGQSSGELRTGGARPPPPLGASS | |
| | QPRPGGDSSPVVDSGPGPASNLTSVPVPHTTALSLPACPEESPLLVGP | |
| | MLIEFNMPVDLELVAKQNPNVKMGGRYAPRDCVSPHKVAIIIPFRN | |
| | RQEHLKYWLYYLHPVLQRQQLDYGIYVINQAGDTIFNRAKLLNVGF | |
| | QEALKDYDYTCFVFSDVDLIPMNDHNAYRCFSQPRHISVAMDKFGF | |
| | SLPYVQYFGGVSALSKQQFLTINGFPNNYWGWGGEDDDIFNRLVFR | |
| | GMSISRPNAVVGRCRMIRHSRDKKNEPNPQRFDRIAHTKETMLSDG | |
| | LNSLTYQVLDVQRYPLYTQITVDIGTPS | |
| 244 | MGYFRCARAGSFGRRRKMEPSTAARAWALFWLLLPLLGAVCASGP | DDOST |
| | RTLVLLDNLNVRETHSLFFRSLKDRGFELTFKTADDPSLSLIKYGEFL | |
| | YDNLIIFSPSVEDFGGNINVETISAFIDGGGSVLVAASSDIGDPLRELG | |
| | SECGIEFDEEKTAVIDHHNYDISDLGQHTLIVADTENLLKAPTIVGKS | |
| | SLNPILFRGVGMVADPDNPLVLDILTGSSTSYSFFPDKPITQYPHAVG | |
| | KNTLLIAGLQARNNARVIFSGSLDFFSDSFFNSAVQKAAPGSQRYSQ | |
| | TGNYELAVALSRWVFKEEGVLRVGPVSHHRVGETAPPNAYTVTDL | |
| | VEYSIVIQQLSNGKWVPFDGDDIQLEFVRIDPFVRTFLKKKGGKYSV | |
| | QFKLPDVYGVFQFKVDYNRLGYTHLYSSTQVSVRPLQHTQYERFIP | |
| | SAYPYYASAFSMMLGLFIFSIVFLHMKEKEKSD | |
| 245 | MTGLYELVWRVLHALLCLHRTLTSWLRVRFGTWNWIWRRCCRAA | NUS1 |
| | SAAVLAPLGFTLRKPPAVGRNRRHHRHPRGGSCLAAAHHRMRWR | |
| | ADGRSLEKLPVHMGLVITEVEQEPSFSDIASLVVWCMAVGISYISVY | |
| | DHQGIFKRNNSRLMDEILKQQQELLGLDCSKYSPEFANSNDKDDQV | |
| | LNCHLAVKVLSPEDGKADIVRAAQDFCQLVAQKQKRPTDLDVDTL | |
| | ASLLSSNGCPDPDLVLKFGPVDSTLGFLPWHIRLTEIVSLPSHLNISYE | |
| | DFFSALRQYAACEQRLGK | |
| 246 | MAPPGSSTVFLLALTIIASTWALTPTHYLTKHDVERLKASLDRPFTN | RPN2 |
| | LESAFYSIVGLSSLGAQVPDAKKACTYIRSNLDPSNVDSLFYAAQAS | |
| | QALSGCEISISNETKDLLLAAVSEDSSVTQIYHAVAALSGFGLPLASQ | |
| | EALSALTARLSKEETVLATVQALQTASHLSQQADLRSIVEEIEDLVA | |
| | RLDELGGVYLQFEEGLETTALFVAATYKLMDHVGTEPSIKEDQVIQ | |
| | LMNAIFSKKNFESLSEAFSVASAAAVLSHNRYHVPVVVVPEGSASD | |
| | THEQAILRLQVTNVLSQPLTQATVKLEHAKSVASRATVLQKTSFTP | |
| | VGDVFELNFMNVKFSSGYYDFLVEVEGDNRYIANTVELRVKISTEV | |
| | GITNVDLSTVDKDQSIAPKTTRVTYPAKAKGTFIADSHQNFALFFQL | |
| | VDVNTGAELTPHQTFVRLHNQKTGQEVVFVAEPDNKNVYKFELDT | |
| | SERKIEFDSASGTYTLYLIIGDATLKNPILWNVADVVIKFPEEEAPST | |
| | VLSQNLFTPKQEIQHLFREPEKRPPTV | |
| | VSNTFTALILSPLLLLFALWIRIGANVSNFTFAPSTIIFHLGHAAMLGL | |
| | MYVYWTQLNMFQTLKYLAILGSVTFLAGNRMLAQQAVKRTAH | |
| 247 | MTTYLEFIQQNEERDGVRFSWNVWPSSRLEATRMVVPVAALFTPLK | SEC23A |
| | ERPDLPPIQYEPVLCSRTTCRAVLNPLCQVDYRAKLWACNFCYQRN | |
| | QFPPSYAGISELNQPAELLPQFSSIEYVVLRGPQMPLIFLYVVDTCME | |
| | DEDLQALKESMQMSLSLLPPTALVGLITFGRMVQVHELGCEGISKS | |
| | YVFRGTKDLSAKQLQEMLGLSKVPLTQATRGPQVQQPPPSNRFLQP | |
| | VQKIDMNLTDLLGELQRDPWPVPQGKRPLRSSGVALSIAVGLLECT | |
| | FPNTGARIMMFIGGPATQGPGM | |
| | VVGDELKTPIRSWHDIDKDNAKYVKKGTKHFEALANRAATTGHVI | |
| | DIYACALDQTGLLEMKCCPNLTGGYMVMGDSFNTSLFKQTFQRVF | |
| | TKDMHGQFKMGFGGTLEIKTSREIKISGAIGPCVSLNSKGPCVSENEI | |
| | GTGGTCQWKICGLSPTTTLAIYFEVVNQHNAPIPQGGRGAIQFVTQY | |
| | QHSSGQRRIRVTTIARNWADAQTQIQNIAASFDQEAAAILMARLAIY | |
| | RAETEEGPDVLRWLDRQLIRLCQKFGEYHKDDPSSFRFSETFSLYPQ | |
| | FMFHLRRSSFLQVFNNSPDESSYYRHHFMRQDLTQSLIMIQPILYAY | |
| | SFSGPPEPVLLDSSSILADRILLMDTFFQILIYHGETIAQWRKSGYQD | |
| | MPEYENFRHLLQAPVDDAQEILHSRFPMPRYIDTEHGGSQARFLLSK | |
| | VNPSQTHNNMYAWGQESGAPILTDDVSLQVFMDHLKKLAVSSAA | |
| 248 | MFANLKYVSLGILVFQTTSLVLTMRYSRTLKEEGPRYLSSTAVVVA | SLC35A3 |
| | ELLKIMACILLVYKDSKCSLRALNRVLHDEILNKPMETLKLAIPSGIY | |
| | TLQNNLLYVALSNLDAATYQVTYQLKILTTALFSVSMLSKKLGVYQ | |
| | WLSLVILMTGVAFVQWPSDSQLDSKELSAGSQFVGLMAVLTACFSS | |
| | GFAGVYFEKILKETKQSVWIRNIQLGFFGSIFGLMGVYIYDGELVSK | |
| | NGFFQGYNRLTWIVVVLQALGGLVIAAVIKYADNILKGFATSLSIILS | |
| | TLISYFWLQDFVPTSVFFLGAILVITATFLYGYDPKPAGNPTKA | |
| 249 | MGLLVFVRNLLLALCLFLVLGFLYYSAWKLHLLQWEEDSNSVVLS | ST3GAL3 |
| | FDSAGQTLGSEYDRLGFLLNLDSKLPAELATKYANFSEGACKPGYA | |
| | SALMTAIFPRFSKPAPMFLDDSFRKWARIREFVPPFGIKGQDNLIKAI | |
| | LSVTKEYRLTPALDSLRCRRCIIVGNGGVLANKSLGSRIDDYDIVVR | |
| | LNSAPVKGFEKDVGSKTTLRITYPEGAMQRPEQYERDSLFVLAGFK | |
| | WQDFKWLKYIVYKERVSASDGFWKSVATRVPKEPPEIRILNPYFIQE | |
| | AAFTLIGLPFNNGLMGRGNIPTLGSVAVTMALHGCDEVAVAGFGY | |
| | DMSTPNAPLHYYETVRMAAIKESWTHNIQREKEFLRKLVKARVITD | |
| | LSSGI | |
| 250 | MTKFGFLRLSYEKQDTLLKLLILSMAAVLSFSTRLFAVLRFESVIHEF | STT3A |
| | DPYFNYRTTRFLAEEGFYKFHNWFDDRAWYPLGRIIGGTIYPGLMIT | |
| | SAAIYHVLHFFHITIDIRNVCVFLAPLFSSFTTIVTYHLTKELKDAGA | |
| | GLLAAAMIAVVPGYISRSVAGSYDNEGIAIFCMLLTYYMWIKAVKT | |
| | GSICWAAKCALAYFYMVSSWGGYVFLINLIPLHVLVLMLTGRFSHR | |
| | IYVAYCTVYCLGTILSMQISFVGFQPVLSSEHMAAFGVFGLCQIHAF | |
| | VDYLRSKLNPQQFEVLFRSVISLVGFVLLTVGALLMLTGKISPWTGR | |
| | FYSLLDPSYAKNNIPIIASVSEHQPTTWSSYYFDLQLLVFMFPVGLYY | |
| | CFSNLSDARIFIIMYGVTSMYFSAVMVRLMLVLAPVMCILSGIGVSQ | |
| | VLSTYMKNLDISRPDKKSKKQQDSTYPIKNEVASGMILVMAFFLITY | |
| | TFHSTWVTSEAYSSPSIVLSARGGDGSRIIFDDFREAYYWLRHNTPE | |
| | DAKVMSWWDYGYQITAMANRTILVDNNTWNNTHISRVGQAMAST | |
| | EEKAYEIMRELDVSYVLVIFGGLTGYSSDDINKFLWMVRIGGSTDT | |
| | GKHIKENDYYTPTGEFRVDREGSPVLLNCLMYKMCYYRFGQVYTE | |
| | AKRPPGFDRVRNAEIGNKDFELDVLEEAYTTEHWLVRIYKVKDLDN | |
| | RGLSRT | |
| 251 | MAEPSAPESKHKSSLNSSPWSGLMALGNSRHGHHGPGAQCAHKAA | STT3B |
| | GGAAPPKPAPAGLSGGLSQPAGWQSLLSFTILFLAWLAGFSSRLFAV | |
| | IRFESIIHEFDPWFNYRSTHHLASHGFYEFLNWFDERAWYPLGRIVG | |
| | GTVYPGLMITAGLIHWILNTLNITVHIRDVCVFLAPTFSGLTSISTFLL | |
| | TRELWNQGAGLLAACFIAIVPGYISRSVAGSFDNEGIAIFALQFTYYL | |
| | WVKSVKTGSVFWTMCCCLSYFYMVSAWGGYVFIINLIPLHVFVLLL | |
| | MQRYSKRVYIAYSTFYIVGLILSMQIPFVGFQPIRTSEHMAAAGVFA | |
| | LLQAYAFLQYLRDRLTKQEFQTLFFLGVSLAAGAVFLSVIYLTYTG | |
| | YIAPWSGRFYSLWDTGYAKIHIPIIASVSEHQPTTWVSFFFDLHILVC | |
| | TFPAGLWFCIKNINDERVFVALYAISAVYFAGVMVRLMLTLTPVVC | |
| | MLSAIAFSNVFEHYLGDDMKRENPPVEDSSDEDDKRNQGNLYDKA | |
| | GKVRKHATEQEKTEEGLGPNIKSIVTMLMLMLLMMFAVHCTWVTS | |
| | NAYSSPSVVLASYNHDGTRNILDDFREAYFWLRQNTDEHARVMSW | |
| | WDYGYQIAGMANRTTLVDNNTWNNSHIALVGKAMSSNETAAYKI | |
| | MRTLDVDYVLVIFGGVIGYSGDDINKFLWMVRIAEGEHPKDIRESD | |
| | YFTPQGEFRVDKAGSPTLLNCLMYKMSYYRFGEMQLDFRTPPGFD | |
| | RTRNAEIGNKDIKFKHLEEAFTSEHWLVRIYKVKAPDNRETLDHKP | |
| | RVTNIFPKQKYLSKKTTKRKRGYIKNKLVFKKGKKISKKTV | |
| 252 | MARKSNLPVLLVPFLLCQALVRCSSPLPLVVNTWPFKNATEAAWR | AGA |
| | ALASGGSALDAVESGCAMCEREQCDGSVGFGGSPDELGETTLDAMI | |
| | MDGTTMDVGAVGDLRRIKNAIGVARKVLEHTTHTLLVGESATTFA | |
| | QSMGFINEDLSTTASQALHSDWLARNCQPNYWRNVIPDPSKYCGPY | |
| | KPPGILKQDIPIHKETEDDRGHDTIGMVVIHKTGHIAAGTSTNGIKFK | |
| | IHGRVGDSPIPGAGAYADDTAGAAAATGNGDILMRFLPSYQAVEY | |
| | MRRGEDPTIACQKVISRIQKHFPEF | |
| | FGAVICANVTGSYGAACNKLSTFTQFSFMVYNSEKNQPTEEKVDCI | |
| 253 | MGAPRSLLLALAAGLAVARPPNIVLIFADDLGYGDLGCYGHPSSTTP | ARSA |
| | NLDQLAAGGLRFTDFYVPVSLCTPSRAALLTGRLPVRMGMYPGVL | |
| | VPSSRGGLPLEEVTVAEVLAARGYLTGMAGKWHLGVGPEGAFLPP | |
| | HQGFHRFLGIPYSHDQGPCQNLTCFPPATPCDGGCDQGLVPIPLLAN | |
| | LSVEAQPPWLPGLEARYMAFAHDLMADAQRQDRPFFLYYASHHTH | |
| | YPQFSGQSFAERSGRGPFGDSLMELDAAVGTLMTAIGDLGLLEETL | |
| | VIFTADNGPETMRMSRGGCSGLLRC | |
| | GKGTTYEGGVREPALAFWPGHIAPGVTHELASSLDLLPTLAALAGA | |
| | PLPNVTLDGFDLSPLLLGTGKSPRQSLFFYPSYPDEVRGVFAVRTGK | |
| | YKAHFFTQGSAHSDTTADPACHASSSLTAHEPPLLYDLSKDPGENY | |
| | NLLGGVAGATPEVLQALKQLQLLKAQLDAAVTFGPSQVARGEDPA | |
| | LQICCHPGCTPRPACCHCPDPHA | |
| 254 | MGPRGAASLPRGPGPRRLLLPVVLPLLLLLLLAPPGSGAGASRPPHL | ARSB |
| | VFLLADDLGWNDVGFHGSRIRTPHLDALAAGGVLLDNYYTQPLCT | |
| | PSRSQLLTGRYQIRTGLQHQIIWPCQPSCVPLDEKLLPQLLKEAGYTT | |
| | HMVGKWHLGMYRKECLPTRRGFDTYFGYLLGSEDYYSHERCTLID | |
| | ALNVTRCALDFRDGEEVATGYKNMYSTNIFTKRAIALITNHPPEKPL | |
| | FLYLALQSVHEPLQVPEEYLKPYDFIQDKNRHHYAGMVSLMDEAV | |
| | GNVTAALKSSGLWNNTVFIFSTDNGGQTLAGGNNWPLRGRKWSL | |
| | WEGGVRGVGFVASPLLKQKGVKNRELIHISDWLPTLVKLARGHTN | |
| | GTKPLDGFDVWKTISEGSPSPRIELLHNIDPNFVDSSPCPRNSMAPAK | |
| | DDSSLPEYSAFNTSVHAAIRHGNWKLLTGYPGCGYWFPPPSQYNVS | |
| | EIPSSDPPTKTLWLFDIDRDPEERHDLSREYPHIVTKLLSRLQFYHKH | |
| | SVPVYFPAQDPRCDPKATGVWGPWM | |
| 255 | MPGRSCVALVLLAAAVSCAVAQHAPPWTEDCRKSTYPPSGPTYRG | ASAH1 |
| | AVPWYTINLDLPPYKRWHELMLDKAPVLKVIVNSLKNMINTFVPSG | |
| | KIMQVVDEKLPGLLGNFPGPFEEEMKGIAAVTDIPLGEIISFNIFYELF | |
| | TICTSIVAEDKKGHLIHGRNMDFGVFLGWNINNDTWVITEQLKPLTV | |
| | NLDFQRNNKTVFKASSFAGYVGMLTGFKPGLFSLTLNERFSINGGY | |
| | LGILEWILGKKDVMWIGFLTRTVLENSTSYEEAKNLLTKTKILAPAY | |
| | FILGGNQSGEGCVITRDRKESLDVYELDAKQGRWYVVQTNYDRWK | |
| | HPFFLDDRRTPAKMCLNRTSQENISFETMYDVLSTKPVLNKLTVYT | |
| | TLIDVTKGQFETYLRDCPDPCIGW | |
| 256 | MSADSSPLVGSTPTGYGTLTIGTSIDPLSSSVSSVRLSGYCGSPWRVI | ATP13A2 |
| | GYHVVVWMMAGIPLLLFRWKPLWGVRLRLRPCNLAHAETLVIEIR | |
| | DKEDSSWQLFTVQVQTEAIGEGSLEPSPQSQAEDGRSQAAVGAVPE | |
| | GAWKDTAQLHKSEEAVSVGQKRVLRYYLFQGQRYIWIETQQAFYQ | |
| | VSLLDHGRSCDDVHRSRHGLSLQDQMVRKAIYGPNVISIPVKSYPQ | |
| | LLVDEALNPYYGFQAFSIALWLADHYYWYALCIFLISSISICLSLYKT | |
| | RKQSQTLRDMVKLSMRVCVCRPGGEEEWVDSSELVPGDCLVLPQE | |
| | GGLMPCDAALVAGECMVNESSLTGESIPVLKTALPEGLGPYCAETH | |
| | RRHTLFCGTLILQARAYVGPHVLAVVTRTGFCTAKGGLVSSILHPRP | |
| | INFKFYKHSMKFVAALSVLALLGTIYSIFILYRNRVPLNEIVIRALDL | |
| | VTVVVPPALPAAMTVCTLYAQSRLRRQGIFCIHPLRINLGGKLQLVC | |
| | FDKTGTLTEDGLDVMGVVPLKGQAFLPLV | |
| | PEPRRLPVGPLLRALATCHALSRLQDTPVGDPMDLKMVESTGWVL | |
| | EEEPAADSAFGTQVLAVMRPPLWEPQLQAMEEPPVPVSVLHRFPFS | |
| | SALQRMSVVVAWPGATQPEAYVKGSPELVAGLCNPETVPTDFAQM | |
| | LQSYTAAGYRVVALASKPLPTVPSLEAAQQLTRDTVEGDLSLLGLL | |
| | VMRNLLKPQTTPVIQALRRTRIRAVMVTGDNLQTAVTVARGCGMV | |
| | APQEHLHVHATHPERGQPASLEFLPMESPTAVNGVKDPDQAASYTV | |
| | EPDPRSRHLALSGPTFGIIVKHFPKL | |
| | LPKVLVQGTVFARMAPEQKTELVCELQKLQYCVGMCGDGANDCG | |
| | ALKAADVGISLSQAEASVVSPFTSSMASIECVPMVIREGRCSLDTSFS | |
| | VFKYMALYSLTQFISVLILYTINTNLGDLQFLAIDLVITTTVAVLMSR | |
| | TGPALVLGRVRPPGALLSVPVLSSLLLQMVLVTGVQLGGYFLTLAQ | |
| | PWFVPLNRTVAAPDNLPNYENTVVFSLSSFQYLILAAAVSKGAPFRR | |
| | PLYTNVPFLVALALLSSVLVGLVLVPGLLQGPLALRNITDTGFKLLL | |
| | LGLVTLNFVGAFMLESVLDQCLPACLRRLRPKRASKKRFKQLEREL | |
| | AEQPWPPLPAGPLR | |
| 257 | MGGCAGSRRRFSDSEGEETVPEPRLPLLDHQGAHWKNAVGFWLLG | CLN3 |
| | LCNNFSYVVMLSAAHDILSHKRTSGNQSHVDPGPTPIPHNSSSRFDC | |
| | NSVSTAAVLLADILPTLVIKLLAPLGLHLLPYSPRVLVSGICAAGSFV | |
| | LVAFSHSVGTSLCGVVFASISSGLGEVTFLSLTAFYPRAVISWWSSG | |
| | TGGAGLLGALSYLGLTQAGLSPQQTLLSMLGIPALLLASYFLLLTSP | |
| | EAQDPGGEEEAESAARQPLIRTEAPESKPGSSSSLSLRERWTVFKGL | |
| | LWYIVPLVVVYFAEYFINQGLFELLFFWNTSLSHAQQYRWYQMLY | |
| | QAGVFASRSSLRCCRIRFTWALALLQCLNLVFLLADVWFGFLPSIYL | |
| | VFLIILYEGLLGGAAYVNTFHNIALETSDEHREFAMAATCISDTLGIS | |
| | LSGLLALPLHDFLCQLS | |
| 258 | MAQEVDTAQGAEMRRGAGAARGRASWCWALALLWLAVVPGWS | CLN5 |
| | RVSGIPSRRHWPVPYKRFDFRPKPDPYCQAKYTFCPTGSPIPVMEGD | |
| | DDIEVFRLQAPVWEFKYGDLLGHLKIMHDAIGFRSTLTGKNYTME | |
| | WYELFQLGNCTFPHLRPEMDAPFWCNQGAACFFEGIDDVHWKENG | |
| | TLVQVATISGNMFNQMAKWVKQDNETGIYYETWNVKASPEKGAE | |
| | TWFDSYDCSKFVLRTFNKLAEFGAEFKNIETNYTRIFLYSGEPTYLG | |
| | NETSVFGPTGNKTLGLAIKRFYYPFKPHLPTKEFLLSLLQIFDAVIVH | |
| | KQFYLFYNFEYWFLPMKFPFIKITYEEIPLPIRNKTLSGL | |
| 259 | MEATRRRQHLGATGGPGAQLGASFLQARHGSVSADEAARTAPFHL | CLN6 |
| | DLWFYFTLQNWVLDFGRPIAMLVFPLEWFPLNKPSVGDYFHMAYN | |
| | VITPFLLLKLIERSPRTLPRSITYVSIIIFIMGASIHLVGDSVNHRLLFSG | |
| | YQHHLSVRENPIIKNLKPETLIDSFELLYYYDEYLGHCMWYIPFFLIL | |
| | FMYFSGCFTASKAESLIPGPALLLVAPSGLYYWYLVTEGQIFILFIFTF | |
| | FAMLALVLHQKRKRLFLDSNGLFLFSSFALTLLLVALWVAWLWND | |
| | PVLRKKYPGVIYVPEPWAFYTLHVSSRH | |
| 260 | MNPASDGGTSESIFDLDYASWGIRSTLMVAGFVFYLGVFVVCHQLS | CLN8 |
| | SSLNATYRSLVAREKVFWDLAATRAVFGVQSTAAGLWALLGDPVL | |
| | HADKARGQQNWCWFHITTATGFFCFENVAVHLSNLIFRTFDLFLVI | |
| | HHLFAFLGFLGCLVNLQAGHYLAMTTLLLEMSTPFTCVSWMLLKA | |
| | GWSESLFWKLNQWLMIHMFHCRMVLTYHMWWVCFWHWDGLVS | |
| | SLYLPHLTLFLVGLALLTLIINPYWTHKKTQQLLNPVDWNFAQPEA | |
| | KSRPEGNGQLLRKKRP | |
| 261 | MIRNWLTIFILFPLKLVEKCESSVSLTVPPVVKLENGSSTNVSLTLRP | CTNS |
| | PLNATLVITFEITFRSKNITILELPDEVVVPPGVTNSSFQVTSQNVGQL | |
| | TVYLHGNHSNQTGPRIRFLVIRSSAISIINQVIGWIYFVAWSISFYPQV | |
| | IMNWRRKSVIGLSFDFVALNLTGFVAYSVFNIGLLWVPYIKEQFLLK | |
| | YPNGVNPVNSNDVFFSLHAVVLTLIIIVQCCLYERGGQRVSWPAIGF | |
| | LVLAWLFAFVTMIVAAVGVTTWLQFLFCFSYIKLAVTLVKYFPQAY | |
| | MNFYYKSTEGWSIGNVLLDFTGGSFSLLQMFLQSYNNDQWTLIFGD | |
| | PTKFGLGVFSIVFDVVFFIQHFCLYRKRPGYDQLN | |
| 262 | MIRAAPPPLFLLLLLLLLLVSWASRGEAAPDQDEIQRLPGLAKQPSF | CTSA |
| | RQYSGYLKGSGSKHLHYWFVESQKDPENSPVVLWLNGGPGCSSLD | |
| | GLLTEHGPFLVQPDGVTLEYNPYSWNLIANVLYLESPAGVGFSYSD | |
| | DKFYATNDTEVAQSNFEALQDFFRLFPEYKNNKLFLTGESYAGIYIP | |
| | TLAVLVMQDPSMNLQGLAVGNGLSSYEQNDNSLVYFAYYHGLLG | |
| | NRLWSSLQTHCCSQNKCNFYDNKDLECVTNLQEVARIVGNSGLNIY | |
| | NLYAPCAGGVPSHFRYEKDTVVVQD | |
| | LGNIFTRLPLKRMWHQALLRSGDKVRMDPPCTNTTAASTYLNNPY | |
| | VRKALNIPEQLPQWDMCNFLVNLQYRRLYRSMNSQYLKLLSSQKY | |
| | QILLYNGDVDMACNFMGDEWFVDSLNQKMEVQRRPWLVKYGDS | |
| | GEQIAGFVKEFSHIAFLTIKGAGHMVPTDKPLAAFTMFSRFLNKQPY | |
| 263 | MQPSSLLPLALCLLAAPASALVRIPLHKFTSIRRTMSEVGGSVEDLIA | CTSD |
| | KGPVSKYSQAVPAVTEGPIPEVLKNYMDAQYYGEIGIGTPPQCFTV | |
| | VFDTGSSNLWVPSIHCKLLDIACWIHHKYNSDKSSTYVKNGTSFDIH | |
| | YGSGSLSGYLSQDTVSVPCQSASSASALGGVKVERQVFG | |
| | EATKQPGITFIAAKFDGILGMAYPRISVNNVLPVFDNLMQQKLVDQ | |
| | NIFSFYLSRDPDAQPGGELMLGGTDSKYYKGSLSYLNVTRKAYWQ | |
| | VHLDQVEVASGLTLCKEGCEAIVDTGTSLMVGPVDEVRELQKAIGA | |
| | VPLIQGEYMIPCEKVSTLPAITLKLGGKGYKLSPEDYTLKVSQAGKT | |
| | LCLSGFMGMDIPPPSGPLWILGDVFIGRYYTVFDRDNNRVGFAEAA | |
| | RL | |
| 264 | MAPWLQLLSLLGLLPGAVAAPAQPRAASFQAWGPPSPELLAPTRFA | CTSF |
| | LEMFNRGRAAGTRAVLGLVRGRVRRAGQGSLYSLEATLEEPPCND | |
| | PMVCRLPVSKKTLLCSFQVLDELGRHVLLRKDCGPVDTKVPGAGEP | |
| | KSAFTQGSAMISSLSQNHPDNRNETFSSVISLLNEDPLSQDLPVKMA | |
| | SIFKNFVITYNRTYESKEEARWRLSVFVNNMVRAQKIQALDRGTAQ | |
| | YGVTKFSDLTEEEFRTIYLNTLLRKEPGNKMKQAKSVGDLAPPEWD | |
| | WRSKGAVTKVKDQGMCGSCWAFSVTGNVEGQWFLNQGTLLSLSE | |
| | QELLDCDKMDKACMGGLPSNAYSAIKNLGGLETEDDYSYQGHMQ | |
| | SCNFSAEKAKVYINDSVELSQNEQKLAAWLAKRGPISVAINAFGMQ | |
| | FYRHGISRPLRPLCSPWLIDHAVLLVGYGNRSDVPFWAIKNSWGTD | |
| | WGEKGYYYLHRGSGACGVNTMASSAVVD | |
| 265 | MWGLKVLLLPVVSFALYPEEILDTHWELWKKTHRKQYNNKVDEIS | CTSK |
| | RRLIWEKNLKYISIHNLEASLGVHTYELAMNHLGDMTSEEVVQKMT | |
| | GLKVPLSHSRSNDTLYIPEWEGRAPDSVDYRKKGYVTPVKNQGQC | |
| | GSCWAFSSVGALEGQLKKKTGKLLNLSPQNLVDCVSENDGCGGGY | |
| | MTNAFQYVQKNRGIDSEDAYPYVGQEESCMYNPTGKAAKCRGYR | |
| | EIPEGNEKALKRAVARVGPVSVAIDASLTSFQFYSKGVYYDESCNSD | |
| | NLNHAVLAVGYGIQKGNKHWIIKNSWGENWGNKGYILMARNKNN | |
| | ACGIANLASFPKM | |
| 266 | MADQRQRSLSTSGESLYHVLGLDKNATSDDIKKSYRKLALKYHPD | DNAJC5 |
| | KNPDNPEAADKFKEINNAHAILTDATKRNIYDKYGSLGLYVAEQFG | |
| | EENVNTYFVLSSWWAKALFVFCGLLTCCYCCCCLCCCFNCCCGKC | |
| | KPKAPEGEETEFYVSPEDLEAQLQSDEREATDTPIVIQPASATETTQL | |
| | TADSHPSYHTDGFN | |
| 267 | MRAPGMRSRPAGPALLLLLLFLGAAESVRRAQPPRRYTPDWPSLDS | FUCA1 |
| | RPLPAWFDEAKFGVFIHWGVFSVPAWGSEWFWWHWQGEGRPQYQ | |
| | RFMRDNYPPGFSYADFGPQFTARFFHPEEWADLFQAAGAKYVVLT | |
| | TKHHEGFTNWPSPVSWNWNSKDVGPHRDLVGELGTALRKRNIRYG | |
| | LYHSLLEWFHPLYLLDKKNGFKTQHFVSAKTMPELYDLVNSYKPD | |
| | LIWSDGEWECPDTYWNSTNFLSWLYNDSPVKDEVVVNDRWGQNC | |
| | SCHHGGYYNCEDKFKPQSLPDHKWEMCTSIDKFSWGYRRDMALSD | |
| | VTEESEIISELVQTVSLGGNYLLNIGPTKDGLIVPIFQERLLAVGK | |
| | WLSINGEAIYASKPWRVQWEKNTTSVWYTSKGSAVYAIFLHWPEN | |
| | GVLNLESPITTSTTKITMLGIQGDLKWSTDPDKGLFISLPQLPPSAVP | |
| | AEFAWTIKLTGVK | |
| 268 | MGVRHPPCSHRLLAVCALVSLATAALLGHILLHDFLLVPRELSGSSP | GAA |
| | VLEETHPAHQQGASRPGPRDAQAHPGRPRAVPTQCDVPPNSRFDCA | |
| | PDKAITQEQCEARGCCYIPAKQGLQGAQMGQPWCFFPPSYPSYKLE | |
| | NLSSSEMGYTATLTRTTPTFFPKDILTLRLDVMMETENRLHFTIKDP | |
| | ANRRYEVPLETPHVHSRAPSPLYSVEFSEEPFGVIVRRQLDGRVLLN | |
| | TTVAPLFFADQFLQLSTSLPSQYITGLAEHLSPLMLSTSWTRITLWNR | |
| | DLAPTPGANLYGSHPFYLALEDGGSAHGVFLLNSNAMDVVLQPSPA | |
| | LSWRSTGGILDVYIFLGPEPKSVVQQYLDVVGYPFMPPYWGLGFHL | |
| | CRWGYSSTAITRQVVENMTRAHFPLDVQWNDLDYMDSRRDFTFN | |
| | KDGFRDFPAMVQELHQGGRRYMMIVDPAISSSGPAGSYRPYDEGLR | |
| | RGVFITNETGQPLIGKVWPGSTAFPDFTNPTALAWWEDMVAEFHD | |
| | QVPFDGMWIDMNEPSNFIRGSEDGCPNNELENPPYVPGVVGGTLQA | |
| | ATICASSHQFLSTHYNLHNLYGLTEAIASHRALVKARGTRPFVISRST | |
| | FAGHGRYAGHWTGDVWSSWEQLASSVPEILQFNLLGVPLVGADVC | |
| | GFLGNTSEELCVRWTQLGAFYPFMRNHNSLLSLPQEPYSFSEPAQQ | |
| | AMRKALTLRYALLPHLYTLFHQAHVAGETVARPLFLEFPKDSSTWT | |
| | VDHQLLWGEALLITPVLQAGKAEVTGYFPLGTWYDLQTVPVEALG | |
| | SLPPPPAAPREPAIHSEGQWVTLPAPLDTINVHLRAGYIIPLQGPGLT | |
| | TTESRQQPMALAVALTKGGEARGELFWDDGESLEVLERGAYTQVIF | |
| | LARNNTIVNELVRVTSEGAGLQLQKVTVLGVATAPQQVLSNGVPVS | |
| | NFTYSPDTKVLDICVSLLMGEQFLVSWC | |
| 269 | MAEWLLSASWQRRAKAMTAAAGSAGRAAVPLLLCALLAPGGAYV | GALC |
| | LDDSDGLGREFDGIGAVSGGGATSRLLVNYPEPYRSQILDYLFKPNF | |
| | GASLHILKVEIGGDGQTTDGTEPSHMHYALDENYFRGYEWWLMKE | |
| | AKKRNPNITLIGLPWSFPGWLGKGFDWPYVNLQLTAYYVVTWIVG | |
| | AKRYHDLDIDYIGIWNERSYNANYIKILRKMLNYQGLQRVKIIASDN | |
| | LWESISASMLLDAELFKVVDVIGAHYPGTHSAKDAKLTGKKLWSSE | |
| | DFSTLNSDMGAGCWGRILNQNYINGYMTSTIAWNLVASYYEQLPY | |
| | GRCGLMTAQEPWSGHYVVESPVWVSAHTTQFTQPGWYYLKTVGH | |
| | LEKGGSYVALTDGLGNLTIIIETMSHKHSKCIRPFLPYFNVSQQFATF | |
| | VLKGSFSEIPELQVWYTKLGKTSERFLFKQLDSLWLLDSDGSFTLSL | |
| | HEDELFTLTTLTTGRKGSYPLPPKSQPFPSTYKDDFNVDYPFFSEAPN | |
| | FADQTGVFEYFTNIEDPGEHHFTLRQVLNQRPITWAADASNTISIIGD | |
| | YNWTNLTIKCDVYIETPDTGGVFIAGRVNKGGILIRSARGIFFWIFAN | |
| | GSYRVTGDLAGWIIYALGRVEVTAKKWYTLTLTIKGHFTSGMLND | |
| | KSLWTDIPVNFPKNGWAAIGTHSFEFAQFDNFLVEATR | |
| 270 | MAAVVAATRWWQLLLVLSAAGMGASGAPQPPNILLLLMDDMGW | GALNS |
| | GDLGVYGEPSRETPNLDRMAAEGLLFPNFYSANPLCSPSRAALLTG | |
| | RLPIRNGFYTTNAHARNAYTPQEIVGGIPDSEQLLPELLKKAGYVSKI | |
| | VGKWHLGHRPQFHPLKHGFDEWFGSPNCHFGPYDNKARPNIPVYR | |
| | DWEMVGRYYEEFPINLKTGEANLTQIYLQEALDFIKRQARHHPFFL | |
| | YWAVDATHAPVYASKPFLGTSQRGRYGDAVREIDDSIGKILELLQD | |
| | LHVADNTFVFFTSDNGAALISAPEQGGSNGPFLCGKQTTFEGGMRE | |
| | PALAWWPGHVTAGQVSHQLGSIMDLFTTSLALAGLTPPSDRAIDGL | |
| | NLLPTLLQGRLMDRPIFYYRGDTLMAATLGQHKAHFWTWTNSWE | |
| | NFRQGIDFCPGQNVSGVTTHNLEDHTKLPLIFHLGRDPGERFPLSFAS | |
| | AEYQEALSRITSVVQQHQEALVPAQPQLNVCNWAVMNWAPPGCE | |
| | KLGKCLTPPESIPKKCLWSH | |
| 271 | MQLRNPELHLGCALALRFLALVSWDIPGARALDNGLARTPTMGWL | GLA |
| | HWERFMCNLDCQEEPDSCISEKLFMEMAELMVSEGWKDAGYEYL | |
| | CIDDCWMAPQRDSEGRLQADPQRFPHGIRQLANYVHSKGLKLGIYA | |
| | DVGNKTCAGFPGSFGYYDIDAQTFADWGVDLLKFDGCYCDSLENL | |
| | ADGYKHMSLALNRTGRSIVYSCEWPLYMWPFQKPNYTEIRQYCNH | |
| | WRNFADIDDSWKSIKSILDWTSFNQERIVDVAGPGGWNDPDMLVIG | |
| | NFGLSWNQQVTQMALWAIMAAPLFMSNDLRHISPQAKALLQDKD | |
| | VIAINQDPLGKQGYQLRQGDNFEVWERPLSGLAWAVAMINRQEIG | |
| | GPRSYTIAVASLGKGVACNPACFITQLLPVKRKLGFYEWTSRLRSHI | |
| | NPTGTVLLQLENTMQMSLKDLL | |
| 272 | MPGFLVRILPLLLVLLLLGPTRGLRNATQRMFEIDYSRDSFLKDGQP | GLB1 |
| | FRYISGSIHYSRVPRFYWKDRLLKMKMAGLNAIQTYVPWNFHEPW | |
| | PGQYQFSEDHDVEYFLRLAHELGLLVILRPGPYICAEWEMGGLPAW | |
| | LLEKESILLRSSDPDYLAAVDKWLGVLLPKMKPLLYQNGGPVITVQ | |
| | VENEYGSYFACDFDYLRFLQKRFRHHLGDDVVLFTTDGAHKTFLK | |
| | CGALQGLYTTVDFGTGSNITDAFLSQRKCEPKGPLINSEFYTGWLDH | |
| | WGQPHSTIKTEAVASSLYDILARG | |
| | ASVNLYMFIGGTNFAYWNGANSPYAAQPTSYDYDAPLSEAGDLTE | |
| | KYFALRNIIQKFEKVPEGPIPPSTPKFAYGKVTLEKLKTVGAALDILC | |
| | PSGPIKSLYPLTFIQVKQHYGFVLYRTTLPQDCSNPAPLSSPLNGVHD | |
| | RAYVAVDGIPQGVLERNNVITLNITGKAGATLDLLVENMGRVNYG | |
| | AYINDFKGLVSNLTLSSNILTDWTIFPLDTEDAVRSHLGGWGHRDSG | |
| | HHDEAWAHNSSNYTLPAFYMGNFSIPSGIPDLPQDTFIQFPGWTKGQ | |
| | VWINGFNLGRYWPARGPQLTLFVPQHILMTSAPNTITVLELEWAPC | |
| | SSDDPELCAVTFVDRPVIGSSVTYDHPSKPVEKRLMPPPPQKNKDS | |
| | WLDHV | |
| 273 | MQSLMQAPLLIALGLLLAAPAQAHLKKPSQLSSFSWDNCDEGKDPA | GM2A |
| | VIRSLTLEPDPIIVPGNVTLSVMGSTSVPLSSPLKVDLVLEKEVAGLW | |
| | IKIPCTDYIGSCTFEHFCDVLDMLIPTGEPCPEPLRTYGLPCHCPFKEG | |
| | TYSLPKSEFVVPDLELPSWLTTGNYRIESVLSSSGKRLGCIKIAASLK | |
| | GI | |
| 274 | MLFKLLQRQTYTCLSHRYGLYVCFLGVVVTIVSAFQFGEVVLEWSR | GNPTAB |
| | DQYHVLFDSYRDNIAGKSFQNRLCLPMPIDVVYTWVNGTDLELLKE | |
| | LQQVREQMEEEQKAMREILGKNTTEPTKKSEKQLECLLTHCIKVPM | |
| | LVLDPALPANITLKDLPSLYPSFHSASDIFNVAKPKNPSTNVSVVVFD | |
| | STKDVEDAHSGLLKGNSRQTVWRGYLTTDKEVPGLVLMQDLAFLS | |
| | GFPPTFKETNQLKTKLPENLSSKVKLLQLYSEASVALLKLNNPKDFQ | |
| | ELNKQTKKNMTIDGKELTISPA | |
| | YLLWDLSAISQSKQDEDISASRFEDNEELRYSLRSIERHAPWVRNIFI | |
| | VTNGQIPSWLNLDNPRVTIVTHQDVFRNLSHLPTFSSPAIESHIHRIEG | |
| | LSQKFIYLNDDVMFGKDVWPDDFYSHSKGQKVYLTWPVPNCAEGC | |
| | PGSWIKDGYCDKACNNSACDWDGGDCSGNSGGSRYIAGGGGTGSI | |
| | GVGQPWQFGGGINSVSYCNQGCANSWLADKFCDQACNVLSCGFD | |
| | AGDCGQDHFHELYKVILLPNQTHYIIPKGECLPYFSFAEVAKRGVEG | |
| | AYSDNPIIRHASIANKWKTIHLIMHSGMNATTIHFNLTFQNTNDEEF | |
| | KMQITVEVDTREGPKLNSTAQKGYENLVSPITLLPEAEILFEDIPKEK | |
| | RFPKFKRHDVNSTRRAQEEVKIPLVNISLLPKDAQLSLNTLDLQLEH | |
| | GDITLKGYNLSKSALLRSFLMNSQHAKIKNQAIITDETNDSLVAPQE | |
| | KQVHKSILPNSLGVSERLQRLTFPAVSVKVNGHDQGQNPPLDLETT | |
| | ARFRVETHTQKTIGGNVTKEKPPSLIVPLESQMTKEKKITGKEKENS | |
| | RMEENAENHIGVTEVLLGRKLQHYTDSYLGFLPWEKKKYFQDLLD | |
| | EEESLKTQLAYFTDSKNTGRQLKDTFADSLRYVNKILNSKFGFTSRK | |
| | VPAHMPHMIDRIVMQELQDMFPEEFDKTSFHKVRHSEDMQFAFSYF | |
| | YYLMSAVQPLNISQVFDEVDTDQSGVLSDREIRTLATRIHELPLSLQ | |
| | DLTGLEHMLINCSKMLPADITQLNNIPPTQESYYDPNLPPVTKSLVT | |
| | NCKPVTDKIHKAYKDKNKYRFEIMGEEEIAFKMIRTNVSHVVGQLD | |
| | DIRKNPRKFVCLNDNIDHNHKDAQTVKAVLRDFYESMFPIPSQFELP | |
| | REYRNRFLHMHELQEWRAYRDKLKFWTHCVLATLIMFTIFSFFAEQ | |
| | LIALKRKIFPRRRIHKEASPNRIRV | |
| 275 | MAAGLARLLLLLGLSAGGPAPAGAAKMKVVEEPNAFGVNNPFLPQ | GNPTG |
| | ASRLQAKRDPSPVSGPVHLFRLSGKCFSLVESTYKYEFCPFHNVTQH | |
| | EQTFRWNAYSGILGIWHEWEIANNTFTGMWMRDGDACRSRSRQSK | |
| | VELACGKSNRLAHVSEPSTCVYALTFETPLVCHPHALLVYPTLPEAL | |
| | QRQWDQVEQDLADELITPQGHEKLLRTLFEDAGYLKTPEENEPTQL | |
| | EGGPDSLGFETLENCRKAHKELSKEIKRLKGLLTQHGIPYTRPTETS | |
| | NLEHLGHETPRAKSPEQLRGDPG | |
| | LRGSL | |
| 276 | MRLLPLAPGRLRRGSPRHLPSCSPALLLLVLGGCLGVFGVAAGTRR | GNS |
| | PNVVLLLTDDQDEVLGGMTPLKKTKALIGEMGMTFSSAYVPSALC | |
| | CPSRASILTGKYPHNHHVVNNTLEGNCSSKSWQKIQEPNTFPAILRS | |
| | MCGYQTFFAGKYLNEYGAPDAGGLEHVPLGWSYWYALEKNSKYY | |
| | NYTLSINGKARKHGENYSVDYLTDVLANVSLDFLDYKSNFEPFFM | |
| | MIATPAPHSPWTAAPQYQKAFQNVFAPRNKNFNIHGTNKHWLIRQ | |
| | AKTPMTNSSIQFLDNAFRKRWQTLLSVD | |
| | DLVEKLVKRLEFTGELNNTYIFYTSDNGYHTGQFSLPIDKRQLYEFD | |
| | IKVPLLVRGPGIKPNQTSKMLVANIDLGPTILDIAGYDLNKTQMDG | |
| | MSLLPILRGASNLTWRSDVLVEYQGEGRNVTDPTCPSLSPGVSQCFP | |
| | DCVCEDAYNNTYACVRTMSALWNLQYCEFDDQEVFVEVYNLTAD | |
| | PDQITNIAKTIDPELLGKMNYRLMMLQSCSGPTCRTPGVFDPGYRFD | |
| | PRLMFSNRGSVRTRRFSKHLL | |
| 277 | MWTLVSWVALTAGLVAGTRCPDGQFCPVACCLDPGGASYSCCRPL | GRN |
| | LDKWPTTLSRHLGGPCQVDAHCSAGHSCIFTVSGTSSCCPFPEAVAC | |
| | GDGHHCCPRGFHCSADGRSCFQRSGNNSVGAIQCPDSQFECPDFST | |
| | CCVMVDGSWGCCPMPQASCCEDRVHCCPHGAFCDLVHTRCITPTG | |
| | THPLAKKLPAQRTNRAVALSSSVMCPDARSRCPDGSTCCELPSGKY | |
| | GCCPMPNATCCSDHLHCCPQDTVCDLIQSKCLSKENATTDLLTKLP | |
| | AHTVGDVKCDMEVSCPDGYTCCRLQSGAWGCCPFTQAVCCEDHIH | |
| | CCPAGFTCDTQKGTCEQGPHQVPWMEKAPAHLSLPDPQALKRDVP | |
| | CDNVSSCPSSDTCCQLTSGEWGCCPIPEAVCCSDHQHCCPQGYTCV | |
| | AEGQCQRGSEIVAGLEKMPARRASLSHPRDIGCDQHTSCPVGQTCC | |
| | PSLGGSWACCQLPHAVCCEDRQHCCPAGYTCNVKARSCEKEVVSA | |
| | QPATFLARSPHVGVKDVECGEGHFCHDNQTCCRDNRQGWACCPY | |
| | RQGVCCADRRHCCPAGFRCAARGTKCLRREAPRWDAPLRDPALRQ | |
| | LL | |
| 278 | MARGSAVAWAALGPLLWGCALGLQGGMLYPQESPSRECKELDGL | GUSB |
| | WSFRADFSDNRRRGFEEQWYRRPLWESGPTVDMPVPSSFNDISQD | |
| | WRLRHFVGWVWYEREVILPERWTQDLRTRVVLRIGSAHSYAIVWV | |
| | NGVDTLEHEGGYLPFEADISNLVQVGPLPSRLRITIAINNTLTPTTLPP | |
| | GTIQYLTDTSKYPKGYFVQNTYFDFFNYAGLQRSVLLYTTPTTYIDD | |
| | ITVTTSVEQDSGLVNYQISVKGSNLFKLEVRLLDAENKVVANGTGT | |
| | QGQLKVPGVSLWWPYLMHERPAYL | |
| | YSLEVQLTAQTSLGPVSDFYTLPVGIRTVAVTKSQFLINGKPFYFHG | |
| | VNKHEDADIRGKGFDWPLLVKDFNLLRWLGANAFRTSHYPYAEEV | |
| | MQMCDRYGIVVIDECPGVGLALPQFFNNVSLHHHMQVMEEVVRR | |
| | DKNHPAVVMWSVANEPASHLESAGYYLKMVIAHTKSLDPSRPVTF | |
| | VSNSNYAADKGAPYVDVICLNSYYSWYHDYGHLELIQLQLATQFE | |
| | NWYKKYQKPIIQSEYGAETIAGFHQDPPLMFTEEYQKSLLEQYHLG | |
| | LDQKRRKYVVGELIWNFADFMTEQSPTRVLGNKKGIFTRQRQPKSA | |
| | AFLLRERYWKIANETRYPHSVAKSQCLENSLFT | |
| 279 | MTSSRLWFSLLLAAAFAGRATALWPWPQNFQTSDQRYVLYPNNFQ | HEXA |
| | FQYDVSSAAQPGCSVLDEAFQRYRDLLFGSGSWPRPYLTGKRHTLE | |
| | KNVLVVSVVTPGCNQLPTLESVENYTLTINDDQCLLLSETVWGALR | |
| | GLETFSQLVWKSAEGTFFINKTEIEDFPRFPHRGLLLDTSRHYLPLSSI | |
| | LDTLDVMAYNKLNVFHWHLVDDPSFPYESFTFPELMRKGSYNPVT | |
| | HIYTAQDVKEVIEYARLRGIRVLAEFDTPGHTLSWGPGIPGLLTPCY | |
| | SGSEPSGTFGPVNPSLNNTYEFMSTFFLEVSSVFPDFYLHLGGDEVD | |
| | FTCWKSNPEIQDFMRKKGFGEDFKQLESFYIQTLLDIVSSYGKGYVV | |
| | WQEVFDNKVKIQPDTIIQVWREDIPVNYMKELELVTKAGFRALLSA | |
| | PWYLNRISYGPDWKDFYIVEPLAFEGTPEQKALVIGGEACMWGEY | |
| | VDNTNLVPRLWPRAGAVAERLWSNKLTSDLTFAYERLSHFRCELLR | |
| | RGVQAQPLNVGFCEQEFEQT | |
| 280 | MELCGLGLPRPPMLLALLLATLLAAMLALLTQVALVVQVAEAARA | HEXB |
| | PSVSAKPGPALWPLPLSVKMTPNLLHLAPENFYISHSPNSTAGPSCTL | |
| | LEEAFRRYHGYIFGFYKWHHEPAEFQAKTQVQQLLVSITLQSECDA | |
| | FPNISSDESYTLLVKEPVAVLKANRVWGALRGLETFSQLVYQDSYG | |
| | TFTINESTIIDSPRFSHRGILIDTSRHYLPVKIILKTLDAMAFNKFNVLH | |
| | WHIVDDQSFPYQSITFPELSNKGSYSLSHVYTPNDVRMVIEYARLRG | |
| | IRVLPEFDTPGHTLSWGKGQKDLLTPCYSRQNKLDSFGPINPTLNTT | |
| | YSFLTTFFKEISEVFPDQFIHLGGDEVEFKCWESNPKIQDFMRQKGF | |
| | GTDFKKLESFYIQKVLDIIATINKGSIVWQEVFDDKAKLAPGTIVEV | |
| | WKDSAYPEELSRVTASGFPVILSAPWYLDLISYGQDWRKYYKVEPL | |
| | DFGGTQKQKQLFIGGEACLWGEYVDATNLTPRLWPRASAVGERLW | |
| | SSKDVRDMDDAYDRLTRHRCRMVERG | |
| | IAAQPLYAGYCNHENM | |
| 281 | MTGARASAAEQRRAGRSGQARAAERAAGMSGAGRALAALLLAAS | HGSNAT |
| | VLSAALLAPGGSSGRDAQAAPPRDLDKKRHAELKMDQALLLIHNE | |
| | LLWTNLTVYWKSECCYHCLFQVLVNVPQSPKAGKPSAAAASVSTQ | |
| | HGSILQLNDTLEEKEVCRLEYRFGEFGNYSLLVKNIHNGVSEIACDL | |
| | AVNEDPVDSNLPVSIAFLIGLAVIIVISFLRLLLSLDDFNNWISKAISSR | |
| | ETDRLINSELGSPSRTDPLDGDVQPATWRLSALPPRLRSVDTFRGIAL | |
| | ILMVFVNYGGGKYWYFKHASWNGLTVADLVFPWFVFIMGSSIFLS | |
| | MTSILQRGCSKFRLLGKIAWRSFLLICIGIIIVNPNYCLGPLSWDKVRI | |
| | PGVLQRLGVTYFVVAVLELLFAKPVPEHCASERSCLSLRDITSSWPQ | |
| | WLLILVLEGLWLGLTFLLPVPGCPTGYLGPGGIGDFGKYPNCTGGA | |
| | AGYIDRLLLGDDHLYQHPSSAVLYHTEVAYDPEGILGTINSIVMAFL | |
| | GVQAGKILLYYKARTKDILIRFTAWCC | |
| | ILGLISVALTKVSENEGFIPVNKNLWSLSYVTTLSSFAFFILLVLYPVV | |
| | DVKGLWTGTPFFYPGMNSILVYVGHEVFENYFPFQWKLKDNQSHK | |
| | EHLTQNIVATALWVLIAYILYRKKIFWKI | |
| 282 | MAAHLLPICALFLTLLDMAQGFRGPLLPNRPFTTVWNANTQWCLE | HYAL1 |
| | RHGVDVDVSVFDVVANPGQTFRGPDMTIFYSSQLGTYPYYTPTGEP | |
| | VFGGLPQNASLIAHLARTFQDILAAIPAPDFSGLAVIDWEAWRPRW | |
| | AFNWDTKDIYRQRSRALVQAQHPDWPAPQVEAVAQDQFQGAARA | |
| | WMAGTLQLGRALRPRGLWGFYGFPDCYNYDFLSPNYTGQCPSGIR | |
| | AQNDQLGWLWGQSRALYPSIYMPAVLEGTGKSQMYVQHRVAEAF | |
| | RVAVAAGDPNLPVLPYVQIFYDTTNHFLPLDELEHSLGESAAQGAA | |
| | GVVLWVSWENTRTKESCQAIKEYMDTTLGPFILNVTSGALLCSQ | |
| | ALCSGHGRCVRRTSHPKALLLLNPASFSIQLTPGGGPLSLRGALSLE | |
| | DQAQMAVEFKCRCYPGWQAPWCERKSMW | |
| 283 | MPPPRTGRGLLWLGLVLSSVCVALGSETQANSTTDALNVLLIIVDDL | IDS |
| | RPSLGCYGDKLVRSPNIDQLASHSLLFQNAFAQQAVCAPSRVSFLTG | |
| | RRPDTTRLYDFNSYWRVHAGNFSTIPQYFKENGYVTMSVGKVFHP | |
| | GISSNHTDDSPYSWSFPPYHPSSEKYENTKTCRGPDGELHANLLCPV | |
| | DVLDVPEGTLPDKQSTEQAIQLLEKMKTSASPFFLAVGYHKPHIPFR | |
| | YPKEFQKLYPLENITLAPDPEVPDGLPPVAYNPWMDIRQREDVQAL | |
| | NISVPYGPIPVDFQRKIRQSYFASVSYLDTQVGRLLSALDDLQLANS | |
| | THAFTSDHGWALGEHGEWAKYSNFDVATHVPLIFYVPGRTASLPEA | |
| | GEKLFPYLDPFDSASQLMEPGRQSMDLVELVSLFPTLAGLAGLQVP | |
| | PRCPVPSFHVELCREGKNLLKHFRFRDLEEDPYLPGNPRELIAYSQY | |
| | PRPSDIPQWNSDKPSLKDIKIMGYSIRTIDYRYTVWVGFNPDEFLAN | |
| | FSDIHAGELYFVDSDPLQDHNMYNDSQGGDLFQLLMP | |
| 284 | MRPLRPRAALLALLASLLAAPPVAPAEAPHLVHVDAARALWPLRRF | IDUA |
| | WRSTGFCPPLPHSQADQYVLSWDQQLNLAYVGAVPHRGIKQVRTH | |
| | WLLELVTTRGSTGRGLSYNFTHLDGYLDLLRENQLLPGFELMGSAS | |
| | GHFTDFEDKQQVFEWKDLVSSLARRYIGRYGLAHVSKWNFETWNE | |
| | PDHHDFDNVSMTMQGFLNYYDACSEGLRAASPALRLGGPGDSFHT | |
| | PPRSPLSWGLLRHCHDGTNFFTGEAGVRLDYISLHRKGARSSISILEQ | |
| | EKVVAQQIRQLFPKFADTPIYNDEADPLVGWSLPQPWRADVTYAA | |
| | MVVKVIAQHQNLLLANTTSAFPYALLSNDNAFLSYHPHPFAQRTLT | |
| | ARFQVNNTRPPHVQLLRKPVLTAMGLLALLDEEQLWAEVSQAGTV | |
| | LDSNHTVGVLASAHRPQGPADAWRAAVLIYASDDTRAHPNRSVAV | |
| | TLRLRGVPPGPGLVYVTRYLDNGLCSPDGEWRRLGRPVFPTAEQFR | |
| | RMRAAEDPVAAAPRPLPAGGRLTLRPALRLPSLLLVHVCARPEKPP | |
| | GQVTRLRALPLTQGQLVLVWSDEHVGSKCLWTYEIQFSQDGKAYT | |
| | PVSRKPSTFNLFVFSPDTGAVSGSYRVRALDYWARPGPFSDPVPYLE | |
| | VPVPRGPPSPGNP | |
| 285 | MVVVTGREPDSRRQDGAMSSSDAEDDFLEPATPTATQAGHALPLLP | KCTD7 |
| | QEFPEVVPLNIGGAHFTTRLSTLRCYEDTMLAAMFSGRHYIPTDSEG | |
| | RYFIDRDGTHFGDVLNFLRSGDLPPRERVRAVYKEAQYYAIGPLLE | |
| | QLENMQPLKGEKVRQAFLGLMPYYKDHLERIVEIARLRAVQRKAR | |
| | FAKLKVCVFKEEMPITPYECPLLNSLRFERSESDGQLFEHHCEVDVS | |
| | FGPWEAVADVYDLLHCLVTDLSAQGLTVDHQCIGVCDKHLVNHY | |
| | YCKRPIYEFKITWW | |
| 286 | MVCFRLFPVPGSGLVLVCLVLGAVRSYALELNLTDSENATCLYAK | LAMP2 |
| | WQMNFTVRYETTNKTYKTVTISDHGTVTYNGSICGDDQNGPKIAV | |
| | QFGPGFSWIANFTKAASTYSIDSVSFSYNTGDNTTFPDAEDKGILTV | |
| | DELLAIRIPLNDLFRCNSLSTLEKNDVVQHYWDVLVQAFVQNGTVS | |
| | TNEFLCDKDKTSTVAPTIHTTVPSPTTTPTPKEKPEAGTYSVNNGND | |
| | TCLLATMGLQLNITQDKVASVININPNTTHSTGSCRSHTALLRLNSS | |
| | TIIKYLDFVFAVKNENRFYLKEVNISMYLVNGSVFSIANNNLSYWDA | |
| | PLGSSYMCNKEQTVSVSGAFQINTFDLRVQPFNVTQGKYSTAQDCS | |
| | ADDDNFLVPIAVGAALAGVLILVLLAYFIGLKHHHAGYEQF | |
| 287 | MGAYARASGVCARGCLDSAGPWTMSRALRPPLPPLCFFLLLLAAA | MAN2B1 |
| | GARAGGYETCPTVQPNMLNVHLLPHTHDDVGWLKTVDQYFYGIK | |
| | NDIQHAGVQYILDSVISALLADPTRRFIYVEIAFFSRWWHQQTNATQ | |
| | EVVRDLVRQGRLEFANGGWVMNDEAATHYGAIVDQMTLGLRFLE | |
| | DTFGNDGRPRVAWHIDPFGHSREQASLFAQMGFDGFFFGRLDYQD | |
| | KWVRMQKLEMEQVWRASTSLKPPTADLFTGVLPNGYNPPRNLCW | |
| | DVLCVDQPLVEDPRSPEYNAKELVDYFLNVATAQGRYYRTNHTVM | |
| | TMGSDFQYENANMWFKNLDKLIRLVNAQQAKGSSVHVLYSTPAC | |
| | YLWELNKANLTWSVKHDDFFPYADGPHQFWTGYFSSRPALKRYER | |
| | LSYNFLQVCNQLEALVGLAANVGPYGSGDSAPLNEAMAVLQHHD | |
| | AVSGTSRQHVANDYARQLAAGWGPCEVLLSNALARLRGFKDHFTF | |
| | CQQLNISICPLSQTAARFQVIVYNPLGRKVNWMVRLPVSEGVFVVK | |
| | DPNGRTVPSDVVIFPSSDSQAHPPELLFSASLPALGFSTYSVAQVPR | |
| | WKPQARAPQPIPRRSWSPALTIENEHIRATFDPDTGLLMEIMNMNQ | |
| | QLLLPVRQTFFWYNASIGDNESDQASGAYIFRPNQQKPLPVSRWAQI | |
| | HLVKTPLVQEVHQNFSAWCSQVVRLYPGQRHLELEWSVGPIPVGD | |
| | TWGKEVISRFDTPLETKGRFYTDSNGREILERRRDYRPTWKLNQTEP | |
| | VAGNYYPVNTRIYITDGNMQLTVLTDRSQGGSSLRDGSLELMVHRR | |
| | LLKDDGRGVSEPLMENGSGAWVRGRHLVLLDTAQAAAAGHRLLA | |
| | EQEVLAPQVVLAPGGGAAYNLGAPPRTQFSGLRRDLPPSVHLLTLA | |
| | SWGPEMVLLRLEHQFAVGEDSGRNLSAPVTLNLRDLFSTFTITRLQE | |
| | TTLVANQLREAASRLKWTTNTGPTPHQTPYQLDPANITLEPMEIRTF | |
| | LASVQWKEVDG | |
| 288 | MRLHLLLLLALCGAGTTAAELSYSLRGNWSICNGNGSLELPGAVPG | MANBA |
| | CVHSALFQQGLIQDSYYRFNDLNYRWVSLDNWTYSKEFKIPFEISK | |
| | WQKVNLILEGVDTVSKILFNEVTIGETDNMFNRYSFDITNVVRDVNS | |
| | IELRFQSAVLYAAQQSKAHTRYQVPPDCPPLVQKGECHVNFVRKEQ | |
| | CSFSWDWGPSFPTQGIWKDVRIEAYNICHLNYFTFSPIYDKSAQEWN | |
| | LEIESTFDVVSSKPVGGQVIVAIPKLQTQQTYSIELQPGKRIVELFVNI | |
| | SKNITVETWWPHGHGNQTGYNMTVLFELDGGLNIEKSAKVYFRTV | |
| | ELIEEPIKGSPGLSFYFKINGFPIFLKGSNWIPADSFQDRVTSELLRLLL | |
| | QSVVDANMNTLRVWGGGIYEQDEFYELCDELGIMVWQDFMFACA | |
| | LYPTDQGFLDSVTAEVAYQIKRLKSHPSIIIWSGNNENEEALMMNW | |
| | YHISFTDRPIYIKDYVTLYVKNIRELVLAGDKSRPFITSSPTNGAETV | |
| | AEAWVSQNPNSNYFGDVHFYDYISDC | |
| | WNWKVFPKARFASEYGYQSWPSFSTLEKVSSTEDWSFNSKFSLHRQ | |
| | HHEGGNKQMLYQAGLHFKLPQSTDPLRTFKDTIYLTQVMQAQCVK | |
| | TETEFYRRSRSEIVDQQGHTMGALYWQLNDIWQAPSWASLEYGGK | |
| | WKMLHYFAQNFFAPLLPVGFENENTFYIYGVSDLHSDYSMTLSVRV | |
| | HTWSSLEPVCSRVTERFVMKGGEAVCLYEEPVSELLRRCGNCTRES | |
| | CVVSFYLSADHELLSPTNYHFLSSPKEAVGLCKAQITAIISQQGDIFV | |
| | FDLETSAVAPFVWLDVGSIPGRFSDNGFLMTEKTRTILFYPWEPTSK | |
| | NELEQSFHVTSLTDIY | |
| 289 | MTAPAGPRGSETERLLTPNPGYGTQAGPSPAPPTPPEEEDLRRRLKY | MCOLN1 |
| | FFMSPCDKFRAKGRKPCKLMLQVVKILVVTVQLILFGLSNQLAVTF | |
| | REENTIAFRHLFLLGYSDGADDTFAAYTREQLYQAIFHAVDQYLAL | |
| | PDVSLGRYAYVRGGGDPWTNGSGLALCQRYYHRGHVDPANDTFDI | |
| | DPMVVTDCIQVDPPERPPPPPSDDLTLLESSSSYKNLTLKFHKLVNV | |
| | TIHFRLKTINLQSLINNEIPDCYTFSVLITFDNKAHSGRIPISLETQAHI | |
| | QECKHPSVFQHGDNSFRLLFDVVVILTCSLSFLLCARSLLRGFLLQN | |
| | EFVGFMWRQRGRVISLWERLEFVNGWYILLVTSDVLTISGTIMKIGI | |
| | EAKNLASYDVCSILLGTSTLLVWVGVIRYLTFFHNYNILIATLRVALP | |
| | SVMRFCCCVAVIYLGYCFCGWIVLGPYHVKFRSLSMVSECLFSLING | |
| | DDMFVTFAAMQAQQGRSSLVWLFSQLYLYSFISLFIYMVLSLFIALI | |
| | TGAYDTIKHPGGAGAEESELQAYIAQCQDSPTSGKFRRGSGSACSLL | |
| | CCCGRDPSEEHSLLVN | |
| 290 | MAGLRNESEQEPLLGDTPGSREWDILETEEHYKSRWRSIRILYLTMF | MFSD8 |
| | LSSVGFSVVMMSIWPYLQKIDPTADTSFLGWVIASYSLGQMVASPIF | |
| | GLWSNYRPRKEPLIVSILISVAANCLYAYLHIPASHNKYYMLVARGL | |
| | LGIGAGNVAVVRSYTAGATSLQERTSSMANISMCQALGFILGPVFQ | |
| | TCFTFLGEKGVTWDVIKLQINMYTTPVLLSAFLGILNIILILAILREHR | |
| | VDDS | |
| | GRQCKSINFEEASTDEAQVPQGNIDQVAVVAINVLFFVTLFIFALFET | |
| | IITPLTMDMYAWTQEQAVLYNGIILAALGVEAVVIFLGVKLLSKKIG | |
| | ERAILLGGLIVVWVGFFILLPWGNQFPKIQWEDLHNNSIPNTTFGEIII | |
| | GLWKSPMEDDNERPTGCSIEQAWCLYTPVIHLAQFLTSAVLIGLGYP | |
| | VCNLMSYTLYSKILGPKPQGVYMGWLTASGSGARILGPMFISQVYA | |
| | HWGPRWAFSLVCGIIVLTITLLGVVYKRLIALSVRYGRIQE | |
| 291 | MLLKTVLLLGHVAQVLMLDNGLLQTPPMGWLAWERFRCNINCDE | NAGA |
| | DPKNCISEQLFMEMADRMAQDGWRDMGYTYLNIDDCWIGGRDAS | |
| | GRLMPDPKRFPHGIPFLADYVHSLGLKLGIYADMGNFTCMGYPGTT | |
| | LDKVVQDAQTFAEWKVDMLKLDGCFSTPEERAQGYPKMAAALNA | |
| | TGRPIAFSCSWPAYEGGLPPRVNYSLLADICNLWRNYDDIQDSWWS | |
| | VLSILNWFVEHQDILQPVAGPGHWNDPDMLLIGNFGLSLEQSRAQM | |
| | ALWTVLAAPLLMSTDLRTISAQNMDILQNPLMIKINQDPLGIQGRRI | |
| | HKEKSLIEVYMRPLSNKASALVFFSCRTDMPYRYHSSLGQLNFTGS | |
| | VIYEAQDVYSGDIISGLRDETNFTVIINPSGVVMWYLYPIKNLEMSQ | |
| | Q | |
| 292 | MEAVAVAAAVGVLLLAGAGGAAGDEAREAAAVRALVARLLGPGP | NAGLU |
| | AADFSVSVERALAAKPGLDTYSLGGGGAARVRVRGSTGVAAAAGL | |
| | HRYLRDFCGCHVAWSGSQLRLPRPLPAVPGELTEATPNRYRYYQN | |
| | VCTQSYSFVWWDWARWEREIDWMALNGINLALAWSGQEAIWQR | |
| | VYLALGLTQAEINEFFTGPAFLAWGRMGNLHTWDGPLPPSWHIKQL | |
| | YLQHRVLDQMRSFGMTPVLPAFAGHVPEAVTRVFPQVNVTKMGS | |
| | WGHFNCSYSCSFLLAPEDPIFPIIGSLFLRELIKEFGTDHIYGADTFNE | |
| | MQPPSSEPSYLAAATTAVYEAMTAVDTEAVWLLQGWLFQHQPQF | |
| | WGPAQIRAVLGAVPRGRLLVLDLFAESQPVYTRTASFQGQPFIWCM | |
| | LHNFGGNHGLFGALEAVNGGPEAARLFPNSTMVGTGMAPEGISQN | |
| | EVVYSLMAELGWRKDPVPDLAAWVTSFAARRYGVSHPDAGAAWR | |
| | LLLRSVYNCSGEACRGHNRSPLVRRPSLQMNTSIWYNRSDVFEAWR | |
| | LLLTSAPSLATSPAFRYDLLDLTRQAVQELVSLYYEEARSAYLSKEL | |
| | ASLLRAGGVLAYELLPALDEVLASDSRFLLGSWLEQARAAAVSEAE | |
| | ADFYEQNSRYQLTLWGPEGNILDYANKQLAGLVANYYTPRWRLFL | |
| | EALVDSVAQGIPFQQHQFDKNVFQLEQAFVLSKQRYPSQPRGDTVD | |
| | LAKKIFLKYYPRWVAGSW | |
| 293 | MTGERPSTALPDRRWGPRILGFWGGCRVWVFAAIFLLLSLAASWSK | NEU1 |
| | AENDFGLVQPLVTMEQLLWVSGRQIGSVDTFRIPLITATPRGTLLAF | |
| | AEARKMSSSDEGAKFIALRRSMDQGSTWSPTAFIVNDGDVPDGLNL | |
| | GAVVSDVETGVVFLFYSLCAHKAGCQVASTMLVWSKDDGVSWST | |
| | PRNLSLDIGTEVFAPGPGSGIQKQREPRKGRLIVCGHGTLERDGVFC | |
| | LLSDDHGASWRYGSGVSGIPYGQPKQENDFNPDECQPYELPDGSVV | |
| | INARNQNNYHCHCRIVLRSYDACDTLRPRDVTFDPELVDPVVAAGA | |
| | VVTSSGIVFFSNPAHPEFRVNLTLRWSFSNGTSWRKET | |
| | VQLWPGPSGYSSLATLEGSMDGEEQAPQLYVLYEKGRNHYTESISV | |
| | AKISVYGTL | |
| 294 | MTARGLALGLLLLLLCPAQVFSQSCVWYGECGIAYGDKRYNCEYS | NPC1 |
| | GPPKPLPKDGYDLVQELCPGFFFGNVSLCCDVRQLQTLKDNLQLPL | |
| | QFLSRCPSCFYNLLNLFCELTCSPRQSQFLNVTATEDYVDPVTNQTK | |
| | TNVKELQYYVGQSFANAMYNACRDVEAPSSNDKALGLLCGKDAD | |
| | ACNATNWIEYMFNKDNGQAPFTITPVFSDFPVHGMEPMNNATKGC | |
| | DESVDEVTAPCSCQDCSIVCGPKPQPPPPPAPWTILGLDAMYVIMWI | |
| | TYMAFLLVFFGAFFAVWCYRKRYFVSEYTPIDSNIAFSVNASDKGE | |
| | ASCCDPVSAAFEGCLRRLFTRWGSFCVRNPGCVIFFSLVFITACSSGL | |
| | VFVRVTTNPVDLWSAPSSQARLEKEYFDQHFGPFFRTEQLIIRAPLT | |
| | DKHIYQPYPSGADVPFGPPLDIQILHQVLDLQIAIENITASYDNETVT | |
| | LQDICLAPLSPYNTNCTILSVLNYFQNSHSVLDHKKGDDFFVYADY | |
| | HTHFLYCVRAPASLNDTSLLHDPCLGTFGGPVFPWLVLGGYDDQN | |
| | YNNATALVITFPVNNYYNDTEKLQRAQAWEKEFINFVKNYKNPNL | |
| | TISFTAERSIEDELNRESDSDVFTVVISYAIMFLYISLALGHMKSCRRL | |
| | LVDSKVSLGIAGILIVLSSVACSLGVFSYIGLPLTLIVIEVIPFLVLAVG | |
| | VDNIFILVQAYQRDERLQGETLDQQLGRVLGEVAPSMFLSSFSETVA | |
| | FFLGALSVMPAVHTFSLFAGLAVFIDFLLQITCFV | |
| | SLLGLDIKRQEKNRLDIFCCVRGAEDGTSVQASESCLFRFFKNSYSPL | |
| | LLKDWMRPIVIAIFVGVLSFSIAVLNKVDIGLDQSLSMPDDSYMVDY | |
| | FKSISQYLHAGPPVYFVLEEGHDYTSSKGQNMVCGGMGCNNDSLV | |
| | QQIFNAAQLDNYTRIGFAPSSWIDDYFDWVKPQSSCCRVDNITDQFC | |
| | NASVVDPACVRCRPLTPEGKQRPQGGDFMRFLPMFLSDNPNPKCG | |
| | KGGHAAYSSAVNILLGHGTRVGATYFMTYHTVLQTSADFIDALKK | |
| | ARLIASNVTETMGINGSAYRVFPYSVFYVFYEQYLTIIDDTIFNLGVS | |
| | LGAIFLVTMVLLGCELWSAVIMCATIAMVLVNMFGVMWLWGISLN | |
| | AVSLVNLVMSCGISVEFCSHITRAFTVSMKGSRVERAEEALAHMGS | |
| | SVFSGITLTKFGGIVVLAFAKSQIFQIFYFRMYLAMVLLGATHGLIFL | |
| | PVLLSYIGPSVNKAKSCATEERYKGTERERLLNF | |
| 295 | MRFLAATFLLLALSTAAQAEPVQFKDCGSVDGVIKEVNVSPCPTQP | NPC2 |
| | CQLSKGQSYSVNVTFTSNIQSKSSKAVVHGILMGVPVPFPIPEPDGC | |
| | KSGINCPIQKDKTYSYLNKLPVKSEYPSIKLVVEWQLQDDKNQSLFC | |
| | WEIPVQIVSHL | |
| 296 | MSCPVPACCALLLVLGLCRARPRNALLLLADDGGFESGAYNNSAIA | SGSH |
| | TPHLDALARRSLLFRNAFTSVSSCSPSRASLLTGLPQHQNGMYGLH | |
| | QDVHHFNSFDKVRSLPLLLSQAGVRTGIIGKKHVGPETVYPFDFAYT | |
| | EENGSVLQVGRNITRIKLLVRKFLQTQDDRPFFLYVAFHDPHRCGHS | |
| | QPQYGTFCEKFGNGESGMGRIPDWTPQAYDPLDVLVPYFVPNTPAA | |
| | RADLAAQYTTVGRMDQGVGLVLQELRDAGVLNDTLVIFTSDNGIPF | |
| | PSGRTNLYWPGTAEPLLVSSPE | |
| | HPKRWGQVSEAYVSLLDLTPTILDWFSIPYPSYAIFGSKTIHLTGRSL | |
| | LPALEAEPLWATVFGSQSHHEVTMSYPMRSVQHRHFRLVHNLNFK | |
| | MPFPIDQDFYVSPTFQDLLNRTTAGQPTGWYKDLRHYYYRARWEL | |
| | YDRSRDPHETQNLATDPRFAQLLEMLRDQLAKWQWETHDPWVCA | |
| | PDGVLEEKLSPQCQPLHNEL | |
| 297 | MASPGCLWLLAVALLPWTCASRALQHLDPPAPLPLVIWHGMGDSC | PPT1 |
| | CNPLSMGAIKKMVEKKIPGIYVLSLEIGKTLMEDVENSFFLNVNSQV | |
| | TTVCQALAKDPKLQQGYNAMGFSQGGQFLRAVAQRCPSPPMINLIS | |
| | VGGQHQGVFGLPRCPGESSHICDFIRKTLNAGAYSKVVQERLVQAE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLG | |
| 298 | MYALFLLASLLGAALAGPVLGLKECTRGSAVWCQNVKTASDCGA | PSAP |
| | VKHCLQTVWNKPTVKSLPCDICKDVVTAAGDMLKDNATEEEILVY | |
| | LEKTCDWLPKPNMSASCKEIVDSYLPVILDIIKGEMSRPGEVCSALN | |
| | LCESLQKHLAELNHQKQLESNKIPELDMTEVVAPFMANIPLLLYPQ | |
| | DGPRSKPQPKDNGDVCQDCIQMVTDIQTAVRTNSTFVQALVEHVK | |
| | EECDRLGPGMADICKNYISQYSEIAIQMMMHMQPKEICALVGFCDE | |
| | VKEMPMQTLVPAKVASKNVIPALELVEPIKKHEVPAKSDVYCEVCE | |
| | FLVKEVTKLIDNNKTEKEILDAFDKMCSKLPKSLSEECQEVVDTYGS | |
| | SILSILLEEVSPELVCSMLHLCSGTRLPALTVHVTQPKDGGFCEVCK | |
| | KLVGYLDRNLEKNSTKQEILAALEKGCSFLPDPYQKQCDQFVAEYE | |
| | PVLIEILVEVMDPSFVCLKIGACPSAHKPLLGTEKCIWGPSYWCQNT | |
| | ETAAQCNAVEHCKRHVWN | |
| 299 | MRSPVRDLARNDGEESTDRTPLLPGAPRAEAAPVCCSARYNLAILA | SLC17A5 |
| | FFGFFIVYALRVNLSVALVDMVDSNTTLEDNRTSKACPEHSAPIKVH | |
| | HNQTGKKYQWDAETQGWILGSFFYGYIITQIPGGYVASKIGGKMLL | |
| | GFGILGTAVLTLFTPIAADLGVGPLIVLRALEGLGEGVTFPAMHAM | |
| | WSSWAPPLERSKLLSISYAGAQLGTVISLPLSGIICYYMNWTYVFYF | |
| | FGTIGIFWFLLWIWLVSDTPQKHKRISHYEKEYILSSLRNQLSSQKSV | |
| | PWVPILKSLPLWAIVVAHFSYNWTFYTLLTLLPTYMKEILRFNVQEN | |
| | GFLSSLPYLGSWLCMILSGQAADNLRAKWNFSTLCVRRIFSLIGMIG | |
| | PAVFLVAAGFIGCDYSLAVAFLTISTTLGGFCSSGFSINHLDIAPSYA | |
| | GILLGITNTFATIPGMVGPVIAKSLTPDNTVGEWQTVFYIAAAINVFG | |
| | AIFFTLFAKGEVQNWALNDHHGHRH | |
| 300 | MPRYGASLRQSCPRSGREQGQDGTAGAPGLLWMGLVLALALALAL | SMPD1 |
| | ALALSDSRVLWAPAEAHPLSPQGHPARLHRIVPRLRDVFGWGNLTC | |
| | PICKGLFTAINLGLKKEPNVARVGSVAIKLCNLLKIAPPAVCQSIVHL | |
| | FEDDMVEVWRRSVLSPSEACGLLLGSTCGHWDIFSSWNISLPTVPKP | |
| | PPKPPSPPAPGAPVSRILFLTDLHWDHDYLEGTDPDCADPLCCRRGS | |
| | GLPPASRPGAGYWGEYSKCDLPLRTLESLLSGLGPAGPFDMVYWT | |
| | GDIPAHDVWHQTRQDQLRALTTVTALVRKFLGPVPVYPAVGNHES | |
| | TPVNSFPPPFIEGNHSSRWLYEAMAKAWEPWLPAEALRTLRIGGFY | |
| | ALSPYPGLRLISLNMNFCSRENFWLLINSTDPAGQLQWLVGELQAA | |
| | EDRGDKVHIIGHIPPGHCLKSWSWNYYRIVARYENTLAAQFFGHTH | |
| | VDEFEVFYDEETLSRPLAVAFLAPSATTYIGLNPGYRVYQIDGNYSG | |
| | SSHVVLDHETYILNLTQANIPGAIPHWQLLYRARETYGLPNTLPTAW | |
| | HNLVYRMRGDMQLFQTFWFLYHKGHPPSEPCGTPCRLATLCAQLS | |
| | ARADSPALCRHLMPDGSLPEAQSLWPRPLFC | |
| 301 | MAAPALGLVCGRCPELGLVLLLLLLSLLCGAAGSQEAGTGAGAGSL | SUMF1 |
| | AGSCGCGTPQRPGAHGSSAAAHRYSREANAPGPVPGERQLAHSKM | |
| | VPIPAGVFTMGTDDPQIKQDGEAPARRVTIDAFYMDAYEVSNTEFE | |
| | KFVNSTGYLTEAEKFGDSFVFEGMLSEQVKTNIQQAVAAAPWWLP | |
| | VKGANWRHPEGPDSTILHRPDHPVLHVSWNDAVAYCTWAGKRLP | |
| | TEAEWEYSCRGGLHNRLFPWGNKLQPKGQHYANIWQGEFPVTNTG | |
| | EDGFQGTAPVDAFPPNGYGLYNIVGNAWEWTSDWWTVHHSVEET | |
| | LNPKGPPSGKDRVKKGGSYMCHRSYCYRYRCAARSQNTPDSSASN | |
| | LGFRCAADRLPTMD | |
| 302 | MGLQACLLGLFALILSGKCSYSPEPDQRRTLPPGWVSLGRADPEEEL | TPP1 |
| | SLTFALRQQNVERLSELVQAVSDPSSPQYGKYLTLENVADLVRPSPL | |
| | TLHTVQKWLLAAGAQKCHSVITQDFLTCWLSIRQAELLLPGAEFHH | |
| | YVGGPTETHVVRSPHPYQLPQALAPHVDFVGGLHRFPPTSSLRQRPE | |
| | PQVTGTVGLHLGVTPSVIRKRYNLTSQDVGSGTSNNSQACAQFLEQ | |
| | YFHDSDLAQFMRLFGGNFAHQASVARVVGQQGRGRAGIEASLDVQ | |
| | YLMSAGANISTWVYSSPGRHEG | |
| | QEPFLQWLMLLSNESALPHVHTVSYGDDEDSLSSAYIQRVNTELMK | |
| | AAARGLTLLFASGDSGAGCWSVSGRHQFRPTFPASSPYVTTVGGTS | |
| | FQEPFLITNEIVDYISGGGFSNVFPRPSYQEEAVTKFLSSSPHLPPSSYF | |
| | NASGRAYPDVAALSDGYWVVSNRVPIPWVSGTSASTPVFGGILSLIN | |
| | EHRILSGRPPLGFLNPRLYQQHGAGLFDVTRGCHESCLDEEVEGQGF | |
| | CSGPGWDPVTGWGTPNFPALLKTLLNP | |
| 303 | MSDKLPYKVADIGLAAWGRKALDIAENEMPGLMRMRERYSASKPL | AHCY |
| | KGARIAGCLHMTVETAVLIETLVTLGAEVQWSSCNIFSTQDHAAAAI | |
| | AKAGIPVYAWKGETDEEYLWCIEQTLYFKDGPLNMILDDGGDLTN | |
| | LIHTKYPQLLPGIRGISEETTTGVHNLYKMMANGILKVPAINVNDSV | |
| | TKSKFDNLYGCRESLIDGIKRATDVMIAGKVAVVAGYGDVGKGCA | |
| | QALRGFGARVIITEIDPINALQAAMEGYEVTTMDEACQEGNIFVTTT | |
| | GCIDIILGRHFEQMKDDAIVCNIG | |
| | HFDVEIDVKWLNENAVEKVNIKPQVDRYRLKNGRRIILLAEGRLVN | |
| | LGCAMGHPSFVMSNSFTNQVMAQIELWTHPDKYPVGVHFLPKKLD | |
| | EAVAEAHLGKLNVKLTKLTEKQAQYLGMSCDGPFKPDHYRY | |
| 304 | MVDSVYRTRSLGVAAEGLPDQYADGEAARVWQLYIGDTRSRTAEY | GNMT |
| | KAWLLGLLRQHGCQRVLDVACGTGVDSIMLVEEGFSVTSVDASDK | |
| | MLKYALKERWNRRHEPAFDKWVIEEANWMTLDKDVPQSAEGGFD | |
| | AVICLGNSFAHLPDCKGDQSEHRLALKNIASMVRAGGLLVIDHRNY | |
| | DHILSTGCAPPGKNIYYKSDLTKDVTTSVLIVNNKAHMVTLDYTVQ | |
| | VPGAGQDGSPGLSKFRLSYYPHCLASFTELLQAAFGGKCQHSVLGD | |
| | FKPYKPGQTYIPCYFIHVLKRTD | |
| 305 | MNGPVDGLCDHSLSEGVFMFTSESVGEGHPDKICDQISDAVLDAHL | MAT1A |
| | KQDPNAKVACETVCKTGMVLLCGEITSMAMVDYQRVVRDTIKHIG | |
| | YDDSAKGFDFKTCNVLVALEQQSPDIAQCVHLDRNEEDVGAGDQG | |
| | LMFGYATDETEECMPLTIILAHKLNARMADLRRSGLLPWLRPDSKT | |
| | QVTVQYMQDNGAVIPVRIHTIVISVQHNEDITLEEMRRALKEQVIRA | |
| | VVPAKYLDEDTVYHLQPSGRFVIGGPQGDAGVTGRKIIVDTYGGW | |
| | GAHGGGAFSGKDYTKVDRSAAYAARWVAKSLVKAGLCRRVLVQ | |
| | VSYAIGVAEPLSISIFTYGTSQKTERELLDVVHKNFDLRPGVIVRDLD | |
| | LKKPIYQKTACYGHFGRSEFPWEVPRKLVF | |
| 306 | MEKGPVRAPAEKPRGARCSNGFPERDPPRPGPSRPAEKPPRPEAKSA | GCH1 |
| | QPADGWKGERPRSEEDNELNLPNLAAAYSSILSSLGENPQRQGLLK | |
| | TPWRAASAMQFFTKGYQETISDVLNDAIFDEDHDEMVIVKDIDMFS | |
| | MCEHHLVPFVGKVHIGYLPNKQVLGLSKLARIVEIYSRRLQVQERL | |
| | TKQIAVAITEALRPAGVGVVVEATHMCMVMRGVQKMNSKTVTST | |
| | MLGVFREDPKTREEFLTLIRS | |
| 307 | MAGKAHRLSAEERDQLLPNLRAVGWNELEGRDAIFKQFHFKDFNR | PCBD1 |
| | AFGFMTRVALQAEKLDHHPEWFNVYNKVHITLSTHECAGLSERDIN | |
| | LASFIEQVAVSMT | |
| 308 | MSTEGGGRRCQAQVSRRISFSASHRLYSKFLSDEENLKLFGKCNNP | PTS |
| | NGHGHNYKVVVTVHGEIDPATGMVMNLADLKKYMEEAIMQPLDH | |
| | KNLDMDVPYFADVVSTTENVAVYIWDNLQKVLPVGVLYKVKVYE | |
| | TDNNIVVYKGE | |
| 309 | MAAAAAAGEARRVLVYGGRGALGSRCVQAFRARNWWVASVDVV | QDPR |
| | ENEEASASIIVKMTDSFTEQADQVTAEVGKLLGEEKVDAILCVAGG | |
| | WAGGNAKSKSLFKNCDLMWKQSIWTSTISSHLATKHLKEGGLLTL | |
| | AGAKAALDGTPGMIGYGMAKGAVHQLCQSLAGKNSGMPPGAAAI | |
| | AVLPVTLDTPMNRKSMPEADFSSWTPLEFLVETFHDWITGKNRPSS | |
| | GSLIQVVTTEGRTELTPAYF | |
| 310 | MEGGLGRAVCLLTGASRGFGRTLAPLLASLLSPGSVLVLSARNDEA | SPR |
| | LRQLEAELGAERSGLRVVRVPADLGAEAGLQQLLGALRELPRPKGL | |
| | QRLLLINNAGSLGDVSKGFVDLSDSTQVNNYWALNLTSMLCLTSSV | |
| | LKAFPDSPGLNRTVVNISSLCALQPFKGWALYCAGKAARDMLFQV | |
| | LALEEPNVRVLNYAPGPLDTDMQQLARETSVDPDMRKGLQELKAK | |
| | GKLVDCKVSAQKLLSLLEKDEFKSGAHVDFYDK | |
| 311 | MDAILNYRSEDTEDYYTLLGCDELSSVEQILAEFKVRALECHPDKHP | DNAJC12 |
| | ENPKAVETFQKLQKAKEILTNEESRARYDHWRRSQMSMPFQQWEA | |
| | LNDSVKTSMHWVVRGKKDLMLEESDKTHTTKMENEECNEQRERK | |
| | KEELASTAEKTEQKEPKPLEKSVSPQNSDSSGFADVNGWHLRFRWS | |
| | KDAPSELLRKFRNYEI | |
| 312 | MLLPAPALRRALLSRPWTGAGLRWKHTSSLKVANEPVLAFTQGSPE | ALDH4A1 |
| | RDALQKALKDLKGRMEAIPCVVGDEEVWTSDVQYQVSPFNHGHK | |
| | VAKFCYADKSLLNKAIEAALAARKEWDLKPIADRAQIFLKAADMLS | |
| | GPRRAEILAKTMVGQGKTVIQAEIDAAAELIDFFRFNAKYAVELEG | |
| | QQPISVPPSTNSTVYRGLEGFVAAISPFNFTAIGGNLAGAPALMGNV | |
| | VLWKPSDTAMLASYAVYRILREAGLPPNIIQFVPADGPLFGDTVTSS | |
| | EHLCGINFTGSVPTFKHLWKQVAQ | |
| | NLDRFHTFPRLAGECGGKNFHFVHRSADVESVVSGTLRSAFEYGGQ | |
| | KCSACSRLYVPHSLWPQIKGRLLEEHSRIKVGDPAEDFGTFFSAVID | |
| | AKSFARIKKWLEHARSSPSLTILAGGKCDDSVGYFVEPCIVESKDPQ | |
| | EPIMKEEIFGPVLSVYVYPDDKYKETLQLVDSTTSYGLTGAVFSQDK | |
| | DVVQEATKVLRNAAGNFYINDKSTGSIVGQQPFGGARASGTNDKP | |
| | GGPHYILRWTSPQVIKETHKPLGDWSYAYMQ | |
| 313 | MALRRALPALRPCIPRFVQLSTAPASREQPAAGPAAVPGGGSATAV | PRODH |
| | RPPVPAVDFGNAQEAYRSRRTWELARSLLVLRLCAWPALLARHEQ | |
| | LLYVSRKLLGQRLFNKLMKMTFYGHFVAGEDQESIQPLLRHYRAFG | |
| | VSAILDYGVEEDLSPEEAEHKEMESCTSAAERDGSGTNKRDKQYQA | |
| | HRAFGDRRNGVISARTYFYANEAKCDSHMETFLRCIEASGRVSDDG | |
| | FIAIKLTALGRPQFLLQFSEVLAKWRCFFHQMAVEQGQAGLAAMDT | |
| | KLEVAVLQESVAKLGIASRAEIEDW | |
| | FTAETLGVSGTMDLLDWSSLIDSRTKLSKHLVVPNAQTGQLEPLLSR | |
| | FTEEEELQMTRMLQRMDVLAKKATEMGVRLMVDAEQTYFQPAISR | |
| | LTLEMQRKFNVEKPLIFNTYQCYLKDAYDNVTLDVELARREGWCF | |
| | GAKLVRGAYLAQERARAAEIGYEDPINPTYEATNAMYHRCLDYVL | |
| | EELKHNAKAKVMVASHNEDTVRFALRRMEELGLHPADHQVYFGQ | |
| | LLGMCDQISFPLGQAGYPVYKYVPYGPVMEVLPYLSRRALENSSLM | |
| | KGTHRERQLLWLELLRRLRTGNLFHRPA | |
| 314 | MTTYSDKGAKPERGRFLHFHSVTFWVGNAKQAASFYCSKMGFEPL | HPD |
| | AYRGLETGSREVVSHVIKQGKIVFVLSSALNPWNKEMGDHLVKHG | |
| | DGVKDIAFEVEDCDYIVQKARERGAKIMREPWVEQDKFGKVKFAV | |
| | LQTYGDTTHTLVEKMNYIGQFLPGYEAPAFMDPLLPKLPKCSLEMI | |
| | DHIVGNQPDQEMVSASEWYLKNLQFHRFWSVDDTQVHTEYSSLRSI | |
| | VVANYEESIKMPINEPAPGKKKSQIQEYVDYNGGAGVQHIALKTEDI | |
| | ITAIRHLRERGLEFLSVPSTYYKQLREKLKTAKIKVKENIDALEELKI | |
| | LVDYDEKGYLLQIFTKPVQDRPTLFLEVIQRHNHQGFGAGNFNSLF | |
| | KAFEEEQNLRGNLTNMETNGVVPGM | |
| 315 | MEFSSPSREECPKPLSRVSIMAGSLTGLLLLQAVSWASGARPCIPKSF | GBA |
| | GYSSVVCVCNATYCDSFDPPTFPALGTFSRYESTRSGRRMELSMGPI | |
| | QANHTGTGLLLTLQPEQKFQKVKGFGGAMTDAAALNILALSPPAQ | |
| | NLLLKSYFSEEGIGYNIIRVPMASCDFSIRTYTYADTPDDFQLHNFSL | |
| | PEEDTKLKIPLIHRALQLAQRPVSLLASPWTSPTWLKTNGAVNGKGS | |
| | LKGQP | |
| | GDIYHQTWARYFVKFLDAYAEHKLQFWAVTAENEPSAGLLSGYPF | |
| | QCLGFTPEHQRDFIARDLGPTLANSTHHNVRLLMLDDQRLLLPHWA | |
| | KVVLTDPEAAKYVHGIAVHWYLDFLAPAKATLGETHRLFPNTMLF | |
| | ASEACVGSKFWEQSVRLGSWDRGMQYSHSIITNLLYHVVGWTDW | |
| | NLALNPEGGPNWVRNFVDSPIIVDITKDTFYKQPMFYHLGHFSKFIP | |
| | EGSQRVGLVASQKNDLDAVALMHPDGSAVVVVLNRSSKDVPLTIK | |
| | DPAVGFLETISPGYSIHTYLWRRQ | |
| 316 | MAELKYISGFGNECSSEDPRCPGSLPEGQNNPQVCPYNLYAEQLSGS | HGD |
| | AFTCPRSTNKRSWLYRILPSVSHKPFESIDEGQVTHNWDEVDPDPNQ | |
| | LRWKPFEIPKASQKKVDFVSGLHTLCGAGDIKSNNGLAIHIFLCNTS | |
| | MENRCFYNSDGDFLIVPQKGNLLIYTEFGKMLVQPNEICVIQRGMRF | |
| | SIDVFEETRGYILEVYGVHFELPDLGPIGANGLANPRDFLIPIAWYED | |
| | RQVPGGYTVINKYQGKLFAAKQDVSPFNVVAWHGNYTPYKYNLK | |
| | NFMVINSVAFDHADPSIFTVLTAKSVRPGVAIADFVIFPPRWGVADK | |
| | TFRPPYYHRNCMSEFMGLIRGHYEAKQGGFLPGGGSLHSTMTPHGP | |
| | DADCFEKASKVKLAPERIADGTMAFMFESSLSLAVTKWGLKASRCL | |
| | DENYHKCWEPLKSHFTPNSRNPAEPN | |
| 317 | MGVLGRVLLWLQLCALTQAVSKLWVPNTDFDVAANWSQNRTPCA | AMN |
| | GGAVEFPADKMVSVLVQEGHAVSDMLLPLDGELVLASGAGFGVSD | |
| | VGSHLDCGAGEPAVFRDSDRFSWHDPHLWRSGDEAPGLFFVDAER | |
| | VPCRHDDVFFPPSASFRVGLGPGASPVRVRSISALGRTFTRDEDLAV | |
| | FLASRAGRLRFHGPGALSVGPEDCADPSGCVCGNAEAQPWICAALL | |
| | QPLGGRCPQAACHSALRPQGQCCDLCGAVVLLTHGPAFDLERYRA | |
| | RILDTFLGLPQYHGLQVAVSKVPRSSRLREADTEIQVVLVENGPETG | |
| | GAGRLARALLADVAENGEALGVLEATMRESGAHVWGSSAAGLAG | |
| | GVAAAVLLALLVLLVAPPLLRRAGRLRWRRHEAAAPAGAPLGFRN | |
| | PVFDVTASEELPLPRRLSLVPKAAADSTSHSYFVNPLFAGAEAEA | |
| 318 | MSGGWMAQVGAWRTGALGLALLLLLGLGLGLEAAASPLSTPTSAQ | CD320 |
| | AAGPSSGSCPPTKFQCRTSGLCVPLTWRCDRDLDCSDGSDEEECRIE | |
| | PCTQKGQCPPPPGLPCPCTGVSDCSGGTDKKLRNCSRLACLAGELR | |
| | CTLSDDCIPLTWRCDGHPDCPDSSDELGCGTNEILPEGDATTMGPPV | |
| | TLESVTSLRNATTMGPPVTLESVPSVGNATSSSAGDQSGSPTAYGVI | |
| | AAAAVLSASLVTATLLLLSWLRAQERLRPLGLLVAMKESLLLSEQK | |
| | TSLP | |
| 319 | MMNMSLPFLWSLLTLLIFAEVNGEAGELELQRQKRSINLQQPRMAT | CUBN |
| | ERGNLVFLTGSAQNIEFRTGSLGKIKLNDEDLSECLHQIQKNKEDIIE | |
| | LKGSAIGLPQNISSQIYQLNSKLVDLERKFQGLQQTVDKKVCSSNPC | |
| | QNGGTCLNLHDSFFCICPPQWKGPLCSADVNECEIYSGTPLSCQNGG | |
| | TCVNTMGSYSCHCPPETYGPQCASKYDDCEGGSVARCVHGICEDL | |
| | MREQAGEPKYSCVCDAGWMFSPNSPACTLDRDECSFQPGPCSTLV | |
| | QCFNTQGSFYCGACPTGWQGNGYICEDINECEINNGGCSVAPPVEC | |
| | VNTPGSSHCQACPPGYQGDGRVCTLTDICSVSNGGCHPDASCSSTL | |
| | GSLPLCTCLPGYTGNGYGPNGCVQLSNICLSHPCLNGQCIDTVSGYF | |
| | CKCDSGWTGVNCTENINECLSNPCLNGGTCVDGVDSFSCECTRLWT | |
| | GALCQVPQQVCGESLSGINGSFSYRSPDVGYVHDVNCFWVIKTEMG | |
| | KVLRITFTFFRLESMDNCPHEFLQVYDGDSSSAFQLGRFCGSSLPHE | |
| | LLSSDNALYFHLYSEHLRNGRGFTVRWETQQPECGGILTGPYGSIKS | |
| | PGYPGNYPPGRDCVWIVVTSPDLLVTFTFGTLSLEHHDDCNKDYLEI | |
| | RDGPLYQDPLLGKFCTTFSVPPLQTTGPFARIHFHSDSQISDQGFHIT | |
| | YLTSPSDLRCGGNYTDPEGELFLPELSGPFTHTRQCVYMMKQPQGE | |
| | QIQINFTHVELQCQSDSSQNYIEVRDGETLLGKVCGNGTISHIKSITN | |
| | SVWIRFKIDASVEKASFRAVYQVACGDELTGEGVIRSPFFPNVYPGE | |
| | RTCRWTIHQPQSQVILLNFTVFEIGSSAHCETDYVEIGSSSILGSPENK | |
| | KYCGTDIPSFITSVYNFLYVTFVKSSSTENHGFMAKFSAEDLACGEIL | |
| | TESTGTIQSPGHPNVYPHGINCTWHILVQPNHLIHLMFETFHLEFHY | |
| | NCTNDYLEVYDTDSETSLGRYCGKSIPPSLTSSGNSL | |
| | MLVFVTDSDLAYEGFLINYEAISAATACLQDYTDDLGTFTSPNFPNN | |
| | YPNNWECIYRITVRTGQLIAVHFTNFSLEEAIGNYYTDFLEIRDGGYE | |
| | KSPLLGIFYGSNLPPTIISHSNKLWLKFKSDQIDTRSGFSAYWDGSST | |
| | GCGGNLTTSSGTFISPNYPMPYYHSSECYWWLKSSHGSAFELEFKDF | |
| | HLEHHPNCTLDYLAVYDGPSSNSHLLTQLCGDEKPPLIRSSGDSMFI | |
| | KLR | |
| | TDEGQQGRGFKAEYRQTCENVVIVNQTYGILESIGYPNPYSENQHC | |
| | NWTIRATTGNTVNYTFLAFDLEHHINCSTDYLELYDGPRQMGRYCG | |
| | VDLPPPGSTTSSKLQVLLLTDGVGRREKGFQMQWFVYGCGGELSG | |
| | ATGSFSSPGFPNRYPPNKECIWYIRTDPGSSIQLTIHDFDVEYHSRCN | |
| | FDVLEIYGGPDFHSPRIAQLCTQRSPENPMQVSSTGNELAIRFKTDLS | |
| | INGRGFNASWQAVTGGCGGIFQAPSGEIHSPNYPSPYRSNTDCSWVI | |
| | RVDRNHRVLLNFTDFDLEPQDSCIMAYDGLSSTMSRLARTCGREQL | |
| | ANPIVSSGNSLFLRFQSGPSRQNRGFRAQFRQACGGHILTSSFDTVSS | |
| | PRFPANYPNNQNCSWIIQAQPPLNHITLSFTHFELERSTTCARDFVEIL | |
| | DGGHEDAPLRGRYCGTDMPHPITSFSSALTLRFVSDSSISAGGFHTT | |
| | VTASVSACGGTFYMAEGIFNSPGYPDIYPPNVECVWNIVSSPGNRLQ | |
| | LSFISFQLEDSQDCSRDFVEIREGNATGHLVGRYCGNSFPLNYSSIVG | |
| | HTLWVRFISDGSGSGTGFQATFMKIFGNDNIVGTHGKVASPFWPEN | |
| | YPHNSNYQWTVNVNASHVVHGRILEMDIEEIQNCYYDKLRIYDGPS | |
| | IHARLIGAYCGTQTESFSSTGNSLTFHFYSDSSISGKGFLLEWFAVDA | |
| | PDGVLPTIAPGACGGFLRTGDAPVFLFSPGWPDSYSNRVDCTWLIQ | |
| | APDSTVELNILSLDIESHRTCAYDSLVIRDGDNNLAQQLAVLCGREIP | |
| | GPIRSTGEYMFIRFTSDSSVTRAGFNASFHKSCGGYLHADRGIITSPK | |
| | YPETYPSNLNCSWHVLVQSGLTIAVHFEQPFQIPNGDSSCNQGDYLV | |
| | LRNGPDICSPPLGPPGGNGHFCGSHASSTLFTSDNQMFVQFISDHSNE | |
| | GQGFKIKYEAKSLACGGNVYIHDADSAGYVTSPNHPHNYPPHADCI | |
| | WILAAPPETRIQLQFEDRFDIEVTPNCTSNYLELRDGVDSDAPILSKF | |
| | CGTSLPSSQWSSGEVMYLRFRSDNSPTHVGFKAKYSIAQCGGRVPG | |
| | QSGVVESIGHPTLPYRDNLFCEWHLQGLSGHYLTISFEDFNLQNSSG | |
| | CEKDFVEIWDNHTSGNILGRYCGNTIPDSIDTSSNTAVVRFVTDGSV | |
| | TASGFRLRFESSMEECGGDLQGSIGTFTSPNYPNPNPHGRICEWRITA | |
| | PEGRRITLMFNNLRLATHPSCNNEHVIVFNGIRSNSPQLEKLCSSVNV | |
| | SNEIKSSGNTMKVIFFTDGSRPYGGFTASYTSSEDAVCGGSLPNTPE | |
| | GNFTSPGYDGVRNYSRNLNCEWTLSNPNQGNSSISIHFEDFYLESHQ | |
| | DCQFDVLEFRVGDADGPLMWRLCGPSKPTLPLVIPYSQVWIHFVTN | |
| | ERVEHIGFHAKYSFTDCGGIQIGDSGVITSPNYPNAYDSLTHCSSLLE | |
| | APQGHTITLTFSDFDIEPHTTCAWDSVTVRNGGSPESPIIGQYCGNSN | |
| | PRTIQSGSNQLVVTFNSDHSLQGGGFYATWNTQTLGCGGIFHSDNG | |
| | TIRSPHWPQNFPENSRCSWTAITHKSKHLEISFDNNFLIPSGDGQCQN | |
| | SFVKVWAGTEEVDKALLATGCGNVAPGPVITPSNTFTAVFQSQEAP | |
| | AQGFSASFVSRCGSNFTGPSGYIISPNYPKQYDNNMNCTYVIEANPL | |
| | SVVLLTFVSFHLEARSAVTGSCVNDGVHIIRGYSVMSTPFATVCG | |
| | DEMPAPLTIAGPVLLNFYSNEQITDFGFKFSYRIISCGGVFNFSSGIITS | |
| | PAYSYADYPNDMHCLYTITVSDDKVIELKFSDFDVVPSTSCSHDYL | |
| | AIYDGANTSDPLLGKFCGSKRPPNVKSSNNSMLLVFKTDSFQTAKG | |
| | WKMSFRQTLGPQQGCGGYLTGSNNTFASPDSDSNGMYDKNLNCV | |
| | WIIIAPVNKVIHLTFNTFALEAASTRQRCLYDYVKLYDGDSENANLA | |
| | GTFCGSTVPAPFISSGNFLTVQFISDLTLEREGFNATYTIMDMPCGGT | |
| | YNATWTPQNISSPNSSDPDVPFSICTWVIDSPPHQQVKITVWALQLT | |
| | SQDCTQNYLQLQDSPQGHGNSRFQFCGRNASAVPVFYSSMSTAMVI | |
| | FKSGVVNRNSRMSFTYQIADCNRDYHKAFGNLRSPGWPDNYDNDK | |
| | DCTVTLTAPQNHTISLFFHSLGIENSVECRNDFLEVRNGSNSNSPLLG | |
| | KYCGTLLPNPVFSQNNELYLRFKSDSVTSDRGYEIIWTSSPSGCGGT | |
| | LYGDRGSFTSPGYPGTYPNNTYCEWVLVAPAGRLVTINFYFISIDDP | |
| | GDCVQNYLTLYDGPNASSPSSGPYCGGDTSIAPFVASSNQVFIKFHA | |
| | DYARRPSAFRLTWDS | |
| 320 | MAWFALYLLSLLWATAGTSTQTQSSCSVPSAQEPLVNGIQVLMENS | GIF |
| | VTSSAYPNPSILIAMNLAGAYNLKAQKLLTYQLMSSDNNDLTIGQL | |
| | GLTIMALTSSCRDPGDKVSILQRQMENWAPSSPNAEASAFYGPSLAI | |
| | LALCQKNSEATLPIAVRFAKTLLANSSPFNVDTGAMATLALTCMYN | |
| | KIPVGSEEGYRSLFGQVLKDIVEKISMKIKDNGIIGDIYSTGLAMQAL | |
| | SVTPEPSKKEWNCKKTTDMILNEIKQGKFHNPMSIAQILPSLKGKTY | |
| | LDVPQVTCSPDHEVQPTLPSNPGPGPTSASNITVIYTINNQLRGVELL | |
| | FNETINVSVKSGSVLLVVLEEAQRKNPMFKFETTMTSWGLVVSSIN | |
| | NIAENVNHKTYWQFLSGVTPLNEGVADYIPFNHEHITANFTQY | |
| 321 | MRQSHQLPLVGLLLFSFIPSQLCEICEVSEENYIRLKPLLNTMIQSNY | TCN1 |
| | NRGTSAVNVVLSLKLVGIQIQTLMQKMIQQIKYNVKSRLSDVSSGE | |
| | LALIILALGVCRNAEENLIYDYHLIDKLENKFQAEIENMEAHNGTPL | |
| | TNYYQLSLDVLALCLFNGNYSTAEVVNHFTPENKNYYFGSQFSVDT | |
| | GAMAVLALTCVKKSLINGQIKADEGSLKNISIYTKSLVEKILSEKKE | |
| | NGLIGN | |
| | TFSTGEAMQALFVSSDYYNENDWNCQQTLNTVLTEISQGAFSNPNA | |
| | AAQVLPALMGKTFLDINKDSSCVSASGNFNISADEPITVTPPDSQSYI | |
| | SVNYSVRINETYFTNVTVLNGSVFLSVMEKAQKMNDTIFGFTMEER | |
| | SWGPYITCIQGLCANNNDRTYWELLSGGEPLSQGAGSYVVRNGENL | |
| | EVRWSKY | |
| 322 | MRHLGAFLFLLGVLGALTEMCEIPEMDSHLVEKLGQHLLPWMDRL | TCN2 |
| | SLEHLNPSIYVGLRLSSLQAGTKEDLYLHSLKLGYQQCLLGSAFSED | |
| | DGDCQGKPSMGQLALYLLALRANCEFVRGHKGDRLVSQLKWFLE | |
| | DEKRAIGHDHKGHPHTSYYQYGLGILALCLHQKRVHDSVVDKLLY | |
| | AVEPFHQGHHSVDTAAMAGLAFTCLKRSNFNPGRRQRITMAIRTVR | |
| | EEILKAQTPEGHFGNVYSTPLALQFLMTSPMRGAELGTACLKARVA | |
| | LLASLQDGAFQNALMISQLLPVLNHKTYIDLIFPDCLAPRVMLEPAA | |
| | ETIPQTQEIISVTLQVLSLLPPYRQSISVLAGSTVEDVLKKAHELGGFT | |
| | YETQASLSGPYLTSVMGKAAGEREFWQLLRDPNTPLLQGIADYRPK | |
| | DGETIELRLVSW | |
| 323 | MQQKTKLFLQALKYSIPHLGKCMQKQHLNHYNFADHCYNRIKLKK | PREPL |
| | YHLTKCLQNKPKISELARNIPSRSFSCKDLQPVKQENEKPLPENMDA | |
| | FEKVRTKLETQPQEEYEIINVEVKHGGFVYYQEGCCLVRSKDEEAD | |
| | NDNYEVLFNLEELKLDQPFIDCIRVAPDEKYVAAKIRTEDSEASTCVI | |
| | IKLSDQPVMEASFPNVSSFEWVKDEEDEDVLFYTFQRNLRCHDVYR | |
| | ATFGDNKRNERFYTEKDPSYFVFLYLTKDSRFLTINIMNKTTSEVWL | |
| | IDGLSPWDPPVLIQKRIHGVLYYVEHRDDELYILTNVGEPTEFKLMR | |
| | TAADTPAIMNWDLFFTMKRNTKVIDLDMFKDHCVLFLKHSNLLYV | |
| | NVIGLADDSVRSLKLPPWACGFIMDTNSDPKNCPFQLCSPIRPPKYY | |
| | TYKFAEGKLFEETGHEDPITKTSRVLRLEAKSKDGKLVPMTVFHKT | |
| | DSEDLQKKPLLVHVYGAYGMDLKMNFRPERRVLVDDGWILAYCH | |
| | VRGGGELGLQWHADGRLTKKLNGLADLEACIKTLHGQGFSQPSLT | |
| | TLTAFSAGGVLAGALCNSNPELVRAVTLEAPFLDVLNTMMDTTLPL | |
| | T | |
| | LEELEEWGNPSSDEKHKNYIKRYCPYQNIKPQHYPSIHITAYENDER | |
| | VPLKGIVSYTEKLKEAIAEHAKDTGEGYQTPNIILDIQPGGNHVIEDS | |
| | HKKITAQIKFLYEELGLDSTSVFEDLKKYLKF | |
| 324 | MAFANLRKVLISDSLDPCCRKILQDGGLQVVEKQNLSKEELIAELQD | PHGDH |
| | CEGLIVRSATKVTADVINAAEKLQVVGRAGTGVDNVDLEAATRKGI | |
| | LVMNTPNGNSLSAAELTCGMIMCLARQIPQATASMKDGKWERKKF | |
| | MGTELNGKTLGILGLGRIGREVATRMQSFGMKTIGYDPIISPEVSASF | |
| | GVQQLPLEEIWPLCDFITVHTPLLPSTTGLLNDNTFAQCKKGVRVVN | |
| | CARGGIVDEGALLRALQSGQCAGAALDVFTEEPPRDRALVDHENVI | |
| | SCPHLGASTKEAQSRCGEEIA | |
| | VQFVDMVKGKSLTGVVNAQALTSAFSPHTKPWIGLAEALGTLMRA | |
| | WAGSPKGTIQVITQGTSLKNAGNCLSPAVIVGLLKEASKQADVNLV | |
| | NAKLLVKEAGLNVTTSHSPAAPGEQGFGECLLAVALAGAPYQAVG | |
| | LVQGTTPVLQGLNGAVFRPEVPLRRDLPLLLFRTQTSDPAMLPTMIG | |
| | LLAEAGVRLLSYQTSLVSDGETWHVMGISSLLPSLEAWKQHVTEAF | |
| | QFHF | |
| 325 | MDAPRQVVNFGPGPAKLPHSVLLEIQKELLDYKGVGISVLEMSHRS | PSAT1 |
| | SDFAKIINNTENLVRELLAVPDNYKVIFLQGGGCGQFSAVPLNLIGL | |
| | KAGRCADYVVTGAWSAKAAEEAKKFGTINIVHPKLGSYTKIPDPST | |
| | WNLNPDASYVYYCANETVHGVEFDFIPDVKGAVLVCDMSSNFLSK | |
| | PVDVSKFGVIFAGAQKNVGSAGVTVVIVRDDLLGFALRECPSVLEY | |
| | KVQAGNSSLYNTPPCFSIYVMGLVLEWIKNNGGAAAMEKLSSIKSQ | |
| | TIYEIIDNSQGFYVCPVEPQNRSKMNIPFRIGNAKGDDALEKRFLDK | |
| | ALELNMLSLKGHRSVGGIRASLYNAVTIEDVQKLAAFMKKFLEMH | |
| | QL | |
| 326 | MVSHSELRKLFYSADAVCFDVDSTVIREEGIDELAKICGVEDAVSE | PSPH |
| | MTRRAMGGAVPFKAALTERLALIQPSREQVQRLIAEQPPHLTPGIRE | |
| | LVSRLQERNVQVFLISGGFRSIVEHVASKLNIPATNVFANRLKFYFN | |
| | GEYAGFDETQPTAESGGKGKVIKLLKEKFHFKKIIMIGDGATDMEA | |
| | CPPADAFIGFGGNVIRQQVKDNAKWYITDFVELLGELEE | |
| 327 | MQRAVSVVARLGFRLQAFPPALCRPLSCAQEVLRRTPLYDFHLAHG | AMT |
| | GKMVAFAGWSLPVQYRDSHTDSHLHTRQHCSLFDVSHMLQTKILG | |
| | SDRVKLMESLVVGDIAELRPNQGTLSLFTNEAGGILDDLIVTNTSEG | |
| | HLYVVSNAGCWEKDLALMQDKVRELQNQGRDVGLEVLDNALLAL | |
| | QGPTAAQVLQAGVADDLRKLPFMTSAVMEVFGVSGCRVTRCGYT | |
| | GEDGVEISVPVAGAVHLATAILKNPEVKLAGLAARDSLRLEAGLCL | |
| | YGNDIDEHTTPVEGSLSWTLGKRRRAAMDFPGAKVIVPQLKGRVQ | |
| | RRRVGLMCEGAPMRAHSPILNMEGTKIGTVTSGCPSPSLKKNVAMG | |
| | YVPCEYSRPGTMLLVEVRRKQQMAVVSKMPFVPTNYYTLK | |
| 328 | MALRVVRSVRALLCTLRAVPSPAAPCPPRPWQLGVGAVRTLRTGP | GCSH |
| | ALLSVRKFTEKHEWVTTENGIGTVGISNFAQEALGDVVYCSLPEVG | |
| | TKLNKQDEFGALESVKAASELYSPLSGEVTEINEALAENPGLVNKSC | |
| | YEDGWLIKMTLSNPSELDELMSEEAYEKYIKSIEE | |
| 329 | MQSCARAWGLRLGRGVGGGRRLAGGSGPCWAPRSRDSSSGGGDS | GLDC |
| | AAAGASRLLERLLPRHDDFARRHIGPGDKDQREMLQTLGLASIDELI | |
| | EKTVPANIRLKRPLKMEDPVCENEILATLHAISSKNQIWRSYIGMGY | |
| | YNCSVPQTILRNLLENSGWITQYTPYQPEVSQGRLESLLNYQTMVC | |
| | DITGLDMANASLLDEGTAAAEALQLCYRHNKRRKFLVDPRCHPQTI | |
| | AVVQTRAKYTGVLTELKLPCEMDFSGKDVSGVLFQYPDTEGKVED | |
| | FTELVERAHQSGSLACCATDLLALC | |
| | ILRPPGEFGVDIALGSSQRFGVPLGYGGPHAAFFAVRESLVRMMPGR | |
| | MVGVTRDATGKEVYRLALQTREQHIRRDKATSNICTAQALLANMA | |
| | AMFAIYHGSHGLEHIARRVHNATLILSEGLKRAGHQLQHDLFFDTL | |
| | KIQCGCSVKEVLGRAAQRQINFRLFEDGTLGISLDETVNEKDLDDLL | |
| | WIFGCESSAELVAESMGEECRGIPGSVFKRTSPFLTHQVFNSYHSET | |
| | NIVRYMKKLENKDISLVHSMIPLGSCTMKLNSSSELAPITWKEFANI | |
| | HPFVPLDQAQGYQQLFRELEKDLCELTGYDQVCFQPNSGAQGEYA | |
| | GLATIRAYLNQKGEGHRTVCLIPKSAHGTNPASAHMAGMKIQPVEV | |
| | DKYGNIDAVHLKAMVDKHKENLAAIMITYPSTNGVFEENISDVCDL | |
| | IHQHGGQVYLDGANMNAQVGICRPGDFGSDVSHLNLHKTFCIPHG | |
| | GGGPGMGPIGVKKHLAPFLPNHPVISLKRNEDACPVGTVSAAPWGS | |
| | SSILPISWAYIKMMGGKGLKQATETAILNANYMAKRLETHYRILFR | |
| | GARGYVGHEFILDTRPFKKSANIEAVDVAKRLQDYGFHAPTMSWP | |
| | VAGTLMVEPTESEDKAELDRFCDAMISIRQEIADIEEGRIDPRVNPLK | |
| | MSPHSLTCVTSSHWDRPYSREVAAFPLPFVKPENKFWPTIARIDDIY | |
| | GDQHLVCTCPPMEVYESPFSEQKRASS | |
| 330 | MSLRCGDAARTLGPRVFGRYFCSPVRPLSSLPDKKKELLQNGPDLQ | LIAS |
| | DFVSGDLADRSTWDEYKGNLKRQKGERLRLPPWLKTEIPMGKNYN | |
| | KLKNTLRNLNLHTVCEEARCPNIGECWGGGEYATATATIMLMGDT | |
| | CTRGCRFCSVKTARNPPPLDASEPYNTAKAIAEWGLDYVVLTSVDR | |
| | DDMPDGGAEHIAKTVSYLKERNPKILVECLTPDFRGDLKAIEKVALS | |
| | GLDVYAHNVETVPELQSKVRDPRANFDQSLRVLKHAKKVQPDVIS | |
| | KTSIMLGLGENDEQVYATMKALREADVDCLTLGQYMQPTRRHLK | |
| | VEEYITPEKFKYWEKVGNELGFHYTASGPLVRSSYKAGEFFL | |
| | KNLVAKRKTKDL | |
| 331 | MAATARRGWGAAAVAAGLRRRFCHMLKNPYTIKKQPLHQFVQRP | NFU1 |
| | LFPLPAAFYHPVRYMFIQTQDTPNPNSLKFIPGKPVLETRTMDFPTPA | |
| | AAFRSPLARQLFRIEGVKSVFFGPDFITVTKENEELDWNLLKPDIYAT | |
| | IMDFFASGLPLVTEETPSGEAGSEEDDEVVAMIKELLDTRIRPTVQE | |
| | DGGDVIYKGFEDGIVQLKLQGSCTSCPSSIITLKNGIQNMLQFYIPEV | |
| | EGVEQVMDDESDEKEANSP | |
| 332 | MSGGDTRAAIARPRMAAAHGPVAPSSPEQVTLLPVQRSFFLPPFSGA | SLC6A9 |
| | TPSTSLAESVLKVWHGAYNSGLLPQLMAQHSLAMAQNGAVPSEAT | |
| | KRDQNLKRGNWGNQIEFVLTSVGYAVGLGNVWRFPYLCYRNGGG | |
| | AFMFPYFIMLIFCGIPLFFMELSFGQFASQGCLGVWRISPMFKGVGY | |
| | GMMVVSTYIGIYYNVVICIAFYYFFSSMTHVLPWAYCNNPWNTHD | |
| | CAGVLDASNLTNGSRPAALPSNLSHLLNHSLQRTSPSEEYWRLYVL | |
| | KLSDDIGNFGEVRLPLLGCLGVSWLVVFLCLIRGVKSSGKVVYFTA | |
| | TFPYVVLTILFVRGVTLEGAFDGIMYYLTPQWDKILEAKVWGDAAS | |
| | QIFYSLGCAWGGLITMASYNKFHNNCYRDSVIISITNCATSVYAGFV | |
| | IFSILGFMANHLGVDVSRVADHGPGLAFVAYPEALTLLPISPLWSLL | |
| | FFFMLILLGLGTQFCLLETLVTAIVDEVGNEWILQKKTYVTLGVAVA | |
| | GFLLGIPLTSQAGIYWLLLMDNYAASFSLVVISCIMCVAIMYIYGHR | |
| | NYFQDIQMMLGFPPPLFFQICWRFVSPAIIFFILVFTVIQYQPITYNHY | |
| | QYPGWAVAIGFLMALSSVLCIPLYAMFRLCRTDGDTLLQRLKNATK | |
| | PSRDWGPALLEHRTGRYAPTIAPSPEDGFEVQPLHPDKAQIPIVGSN | |
| | GSSRLQDSRI | |
| 333 | MEPSSKKLTGRLMLAVGGAVLGSLQFGYNTGVINAPQKVIEEFYNQ | SLC2A1 |
| | TWVHRYGESILPTTLTTLWSLSVAIFSVGGMIGSFSVGLFVNRFGRR | |
| | NSMLMMNLLAFVSAVLMGFSKLGKSFEMLILGRFIIGVYCGLTTGF | |
| | VPMYVGEVSPTALRGALGTLHQLGIVVGILIAQVFGLDSIMGNKDL | |
| | WPLLLSIIFIPALLQCIVLPFCPESPRFLLINRNEENRAKSVLKKLRGT | |
| | ADVTHDLQEMKEESRQMMREKKVTILELFRSPAYRQPILIAVVLQL | |
| | SQQLSGINAVFYYSTSIFEKAGVQQPVYATIGSGIVNTAFTVVSLFVV | |
| | ERAGRRTLHLIGLAGMAGCAILMTIALALLEQLPWMSYLSIVAIFGF | |
| | VAFFEVGPGPIPWFIVAELFSQGPRPAAIAVAGFSNWTSNFIVGMCF | |
| | QYVEQLCGPYVFIIFTVLLVLFFIFTYFKVPETKGRTFDEIASGFRQG | |
| | GASQSDKTPE | |
| | ELFHPLGADSQV | |
| 334 | MDPSMGVNSVTISVEGMTCNSCVWTIEQQIGKVNGVHHIKVSLEEK | ATP7A |
| | NATIIYDPKLQTPKTLQEAIDDMGFDAVIHNPDPLPVLTDTLFLTVTA | |
| | SLTLPWDHIQSTLLKTKGVTDIKIYPQKRTVAVTIIPSIVNANQIKELV | |
| | PELSLDTGTLEKKSGACEDHSMAQAGEVVLKMKVEGMTCHSCTST | |
| | IEGKIGKLQGVQRIKVSLDNQEATIVYQPHLISVEEMKKQIEAMGFP | |
| | AFVKKQPKYLKLGAIDVERLKNTPVKSSEGSQQRSPSYTNDSTATFII | |
| | DGMHCKSCVSNIESTLSALQYVSSIVVSLENRSAIVKYNASSVTPESL | |
| | RKAIEAVSPGLYRVSITSEVESTSNSPSSSSLQKIPLNVVSQPLTQETV | |
| | INIDGMTCNSCVQSIEGVISKKPGVKSIRVSLANSNGTVEYDPLLTSP | |
| | ETLRGAIEDMGFDATLSDTNEPLVVIAQPSSEMPLLTSTNEFYTKGM | |
| | TPVQD | |
| | KEEGKNSSKCYIQVTGMTCASCVANIERNLRREEGIYSILVALMAG | |
| | KAEVRYNPAVIQPPMIAEFIRELGFGATVIENADEGDGVLELVVRG | |
| | MTCASCVHKIESSLTKHRGILYCSVALATNKAHIKYDPEIIGPRDIIHT | |
| | IESLGFEASLVKKDRSASHLDHKREIRQWRRSFLVSLFFCIPVMGLMI | |
| | YMMVMDHHFATLHHNQNMSKEEMINLHSSMFLERQILPGLSVMNL | |
| | LSFLLC | |
| | VPVQFFGGWYFYIQAYKALKHKTANMDVLIVLATTIAFAYSLIILLV | |
| | AMYERAKVNPITFFDTPPMLFVFIALGRWLEHIAKGKTSEALAKLIS | |
| | LQATEATIVTLDSDNILLSEEQVDVELVQRGDIIKVVPGGKFPVDGR | |
| | VIEGHSMVDESLITGEAMPVAKKPGSTVIAGSINQNGSLLICATHVG | |
| | ADTTLSQIVKLVEEAQTSKAPIQQFADKLSGYFVPFIVFVSIATLLVW | |
| | IVIG | |
| | FLNFEIVETYFPGYNRSISRTETIIRFAFQASITVLCIACPCSLGLATPT | |
| | AVMVGTGVGAQNGILIKGGEPLEMAHKVKVVVFDKTGTITHGTPV | |
| | VNQVKVLTESNRISHHKILAIVGTAESNSEHPLGTAITKYCKQELDTE | |
| | TLGTCIDFQVVPGCGISCKVTNIEGLLHKNNWNIEDNNIKNASLVQI | |
| | DASNEQSSTSSSMIIDAQISNALNAQQYKVLIGNREWMIRNGLVINN | |
| | DVN | |
| | DFMTEHERKGRTAVLVAVDDELCGLIAIADTVKPEAELAIHILKSMG | |
| | LEVVLMTGDNSKTARSIASQVGITKVFAEVLPSHKVAKVKQLQEEG | |
| | KRVAMVGDGINDSPALAMANVGIAIGTGTDVAIEAADVVLIRNDLL | |
| | DVVASIDLSRKTVKRIRINFVFALIYNLVGIPIAAGVFMPIGLVLQPW | |
| | MGSAAMAASSVSVVLSSLFLKLYRKPTYESYELPARSQIGQKSPSEI | |
| | SVHVGIDDTSRNSPKLGLLDRIVNYSRASINSLLSDKRSLNSVVTSEP | |
| | DKHSLLVGDFREDDDTAL | |
| 335 | MMRFMLLFSRQGKLRLQKWYLATSDKERKKMVRELMQVVLARKP | AP1S1 |
| | KMCSFLEWRDLKVVYKRYASLYFCCAIEGQDNELITLELIHRYVEL | |
| | LDKYFGSVCELDIIFNFEKAYFILDEFLMGGDVQDTSKKSVLKAIEQ | |
| | ADLLQEEDESPRSVLEEMGLA | |
| 336 | MKILILGIFLFLCSTPAWAKEKHYYIGIIETTWDYASDHGEKKLISVD | CP |
| | TEHSNIYLQNGPDRIGRLYKKALYLQYTDETFRTTIEKPVWLGFLGPI | |
| | IKAETGDKVYVHLKNLASRPYTFHSHGITYYKEHEGAIYPDNTTDF | |
| | QRADDKVYPGEQYTYMLLATEEQSPGEGDGNCVTRIYHSHIDAPK | |
| | DIASGLIGPLIICKKDSLDKEKEKHIDREFVVMFSVVDENFSWYLED | |
| | NIKTYC | |
| | SEPEKVDKDNEDFQESNRMYSVNGYTFGSLPGLSMCAEDRVKWYL | |
| | FGMGNEVDVHAAFFHGQALTNKNYRIDTINLFPATLFDAYMVAQN | |
| | PGEWMLSCQNLNHLKAGLQAFFQVQECNKSSSKDNIRGKHVRHYY | |
| | IAAEEIIWNYAPSGIDIFTKENLTAPGSDSAVFFEQGTTRIGGSYKKL | |
| | VYREYTDASFTNRKERGPEEEHLGILGPVIWAEVGDTIRVTFHNKG | |
| | AYPLSIEPIGVRFNKNNEGTYYSPNYNPQSRSVPPSASHVAPTETFTY | |
| | EWTVPKEVGPTNADPVCLAKMYY | |
| | SAVDPTKDIFTGLIGPMKICKKGSLHANGRQKDVDKEFYLFPTVFDE | |
| | NESLLLEDNIRMFTTAPDQVDKEDEDFQESNKMHSMNGFMYGNQP | |
| | GLTMCKGDSVVWYLFSAGNEADVHGIYFSGNTYLWRGERRDTAN | |
| | LFPQTSLTLHMWPDTEGTFNVECLTTDHYTGGMKQKYTVNQCRRQ | |
| | SEDSTFYLGERTYYIAAVEVEWDYSPQREWEKELHHLQEQNVSNAF | |
| | LDKGEFYIGSKYKKVVYRQYTDSTFRVPVERKAEEEHLGILGPQLH | |
| | ADVGDKVKIIFKNMATRPYSIHAHGVQTESSTVTPTLPGETLTYVW | |
| | KIPERSGAGTEDSACIPWAYYSTVDQVKDLYSGLIGPLIVCRRPYLK | |
| | VFNPRRKLEFALLFLVFDENESWYLDDNIKTYSDHPEKVNKDDEEFI | |
| | ESNKMHAINGRMFGNLQGLTMHVGDEVNWYLMGMGNEIDLHTV | |
| | HFHGHSFQYKHRGVYSSDVFDIFPGTYQTLEMFPRTPGIWLLHCHV | |
| | TDHIHAGMETTYTVLQNEDTKSG | |
| 337 | MSPTISHKDSSRQRRPGNFSHSLDMKSGPLPPGGWDDSHLDSAGRE | SLC33A1 |
| | GDREALLGDTGTGDFLKAPQSFRAELSSILLLLFLYVLQGIPLGLAGS | |
| | IPLILQSKNVSYTDQAFFSFVFWPFSLKLLWAPLVDAVYVKNFGRRK | |
| | SWLVPTQYILGLFMIYLSTQVDRLLGNTDDRTPDVIALTVAFFLFEF | |
| | LAATQDIAVDGWALTMLSRENVGYASTCNSVGQTAGYFLGNVLFL | |
| | ALESADFCNKYLRFQPQPRGIVTLSDFLFFWGTVFLITTTLVALLKK | |
| | ENEVSVVKEETQGITDTYKL | |
| | LFAIIKMPAVLTFCLLILTAKIGFSAADAVTGLKLVEEGVPKEHLALL | |
| | AVPMVPLQIILPLIISKYTAGPQPLNTFYKAMPYRLLLGLEYALLVW | |
| | WTPKVEHQGGFPIYYYIVVLLSYALHQVTVYSMYVSIMAFNAKVS | |
| | DPLIGGTYMTLLNTVSNLGGNWPSTVALWLVDPLTVKECVGASNQ | |
| | NCRTPDAVELCKKLGGSCVTALDGYYVESIICVFIGFGWWFFLGPKF | |
| | KKLQDEGSSSWKCKRNN | |
| 338 | MSAVCGGAARMLRTPGRHGYAAEFSPYLPGRLACATAQHYGIAGC | PEX7 |
| | GTLLILDPDEAGLRLFRSFDWNDGLFDVTWSENNEHVLITCSGDGSL | |
| | QLWDTAKAAGPLQVYKEHAQEVYSVDWSQTRGEQLVVSGSWDQT | |
| | VKLWDPTVGKSLCTFRGHESIIYSTIWSPHIPGCFASASGDQTLRIWD | |
| | VKAAGVRIVIPAHQAEILSCDWCKYNENLLVTGAVDCSLRGWDLR | |
| | NVRQPVFELLGHTYAIRRVKFSPFHASVLASCSYDFTVRFWNFSKPD | |
| | SLLETVEHHTEFTCGLDFSLQSPTQVADCSWDETIKIYDPACLTIPA | |
| 339 | MEQLRAAARLQIVLGHLGRPSAGAVVAHPTSGTISSASFHPQQFQY | PHYH |
| | TLDNNVLTLEQRKFYEENGFLVIKNLVPDADIQRFRNEFEKICRKEV | |
| | KPLGLTVMRDVTISKSEYAPSEKMITKVQDFQEDKELFRYCTLPEIL | |
| | KYVECFTGPNIMAMHTMLINKPPDSGKKTSRHPLHQDLHYFPFRPS | |
| | DLIVCAWTAMEHISRNNGCLVVLPGTHKGSLKPHDYPKWEGGVNK | |
| | MFHGIQDYEENKARVHLVMEKGDTVFFHPLLIHGSGQNKTQGFRK | |
| | AISCHFASADCHYIDVKGTSQENIEKEVVGIAHKFFGAENSVNLKDI | |
| | WMFRARLVKGERTNL | |
| 340 | MAEAAAAAGGTGLGAGASYGSAADRDRDPDPDRAGRRLRVLSGH | AGPS |
| | LLGRPREALSTNECKARRAASAATAAPTATPAAQESGTIPKKRQEV | |
| | MKWNGWGYNDSKFIFNKKGQIELTGKRYPLSGMGLPTFKEWIQNT | |
| | LGVNVEHKTTSKASLNPSDTPPSVVNEDFLHDLKETNISYSQEADDR | |
| | VFRAHGHCLHEIFLLREGMFERIPDIVLWPTCHDDVVKIVNLACKY | |
| | NLCIIPIGGGTSVSYGLMCPADETRTIISLDTSQMNRILWVDENNLTA | |
| | HVEAGITGQELERQLKESGYCTGH | |
| | EPDSLEFSTVGGWVSTRASGMKKNIYGNIEDLVVHIKMVTPRGIIEK | |
| | SCQGPRMSTGPDIHHFIMGSEGTLGVITEATIKIRPVPEYQKYGSVAF | |
| | PNFEQGVACLREIAKQRCAPASIRLMDNKQFQFGHALKPQVSSIFTS | |
| | FLDGLKKFYITKFKGFDPNQLSVATLLFEGDREKVLQHEKQVYDIA | |
| | AKFGGLAAGEDNGQRGYLLTYVIAYIRDLALEYYVLGESFETSAPW | |
| | DRVVDLCRNVKERITRECKEKGVQFAPFSTCRVTQTYDAGACIYFY | |
| | FAFNYRGISDPLTVFEQTEAAAREEILANGGSLSHHHGVGKLRKQW | |
| | LKESISDVGFGMLKSVKEYVDPNNIFGNRNLL | |
| 341 | MESSSSSNSYFSVGPTSPSAVVLLYSKELKKWDEFEDILEERRHVSD | GNPAT |
| | LKFAMKCYTPLVYKGITPCKPIDIKCSVLNSEEIHYVIKQLSKESLQS | |
| | VDVLREEVSEILDEMSHKLRLGAIRFCAFTLSKVFKQIFSKVCVNEE | |
| | GIQKLQRAIQEHPVVLLPSHRSYIDFLMLSFLLYNYDLPVPVIAAGM | |
| | DFLGMKMVGELLRMSGAFFMRRTFGGNKLYWAVFSEYVKTMLRN | |
| | GYAPVEFFLEGTRSRSAKTLTPKFGLLNIVMEPFFKREVFDTYLVPIS | |
| | ISYDKILEETLYVYELLGVPKPKESTTGLLKARKILSENFGSIHVYFG | |
| | DPVSLRSLAAGRMSRSSYNLVPRYIPQKQSEDMHAFVTEVAYKMEL | |
| | LQIENMVLSPWTLIVAVLLQNRPSMDFDALVEKTLWLKGLTQAFGG | |
| | FLIWPDNKPAEEVVPASILLHSNIASLVKDQVILKVDSGDSEVVDGL | |
| | MLQHITLLMCSAYRNQLLNIFVRPSLVAVALQMTPGFRKEDVYSCF | |
| | RFLRDVFADEFIFLPGNTLKDFEEGCYLLCKSEAIQVTTKDILVTEKG | |
| | NTVLEFLVGLFKPFVESYQIICKYLLSEEEDHFSEEQYLAAVRKFTSQ | |
| | LLDQGTSQCYDVLSSDVQKNALAACVRLGVVEKKKINNNCIFNVN | |
| | EPATTKLEEMLGCKTPIGKPATAKL | |
| 342 | MPVLSRPRPWRGNTLKRTAVLLALAAYGAHKVYPLVRQCLAPARG | ABCD1 |
| | LQAPAGEPTQEASGVAAAKAGMNRVFLQRLLWLLRLLFPRVLCRE | |
| | TGLLALHSAALVSRTFLSVYVARLDGRLARCIVRKDPRAFGWQLLQ | |
| | WLLIALPATFVNSAIRYLEGQLALSFRSRLVAHAYRLYFSQQTYYRV | |
| | SNMDGRLRNPDQSLTEDVVAFAASVAHLYSNLTKPLLDVAVTSYT | |
| | LLRAARSRGAGTAWPSAIAGLVVFLTANVLRAFSPKFGELVAEEAR | |
| | RKGELRYMHSRVVANSEEIAFYGGHEVELALLQRSYQDLASQINLIL | |
| | LERLWYVMLEQFLMKYVWSASGLLMVAVPIITATGYSESDAEAVK | |
| | KAALEKKEEELVSERTEAFTIARNLLTAAADAIERIMSSYKEVTELA | |
| | GYTARVHEMFQVFEDVQRCHFKRPRELEDAQAGSGTIGRSGVRVE | |
| | GPLKIRGQVVDVEQGIICENIPIVTPSGEVVVASLNIRVEEGMHLLITG | |
| | PNGCGKSSLFRILGGLWPTYGGVLYKPPPQRMFYIPQRPYMSVGSL | |
| | RDQVIYPDSVEDMQRKGYSEQDLEAILDVVHLHHILQREGGWEAM | |
| | CD | |
| | WKDVLSGGEKQRIGMARMFYHRPKYALLDECTSAVSIDVEGKIFQ | |
| | AAKDAGIALLSITHRPSLWKYHTHLLQFDGEGGWKFEKLDSAARLS | |
| | LTEEKQRLEQQLAGIPKMQRRLQELCQILGEAVAPAHVPAPSPQGP | |
| | GGLQGAST | |
| 343 | MNPDLRRERDSASFNPELLTHILDGSPEKTRRRREIENMILNDPDFQ | ACOX1 |
| | HEDLNFLTRSQRYEVAVRKSAIMVKKMREFGIADPDEIMWFKKLHL | |
| | VNFVEPVGLNYSMFIPTLLNQGTTAQKEKWLLSSKGLQIIGTYAQTE | |
| | MGHGTHLRGLETTATYDPETQEFILNSPTVTSIKWWPGGLGKTSNH | |
| | AIVLAQLITKGKCYGLHAFIVPIREIGTHKPLPGITVGDIGPKFGYDEI | |
| | DNGYLKMDNHRIPRENMLMKYAQVKPDGTYVKPLSNKLTYGTMV | |
| | FVRSFLVGEAARALSKACTIAIRYSAVRHQSEIKPGEPEPQILDFQTQ | |
| | QYKLFPLLATAYAFQFVGAYMKETYHRINEGIGQGDLSELPELHAL | |
| | TAGLKAFTSWTANTGIEACRMACGGHGYSHCSGLPNIYVNFTPSCT | |
| | FEGENTVMMLQTARFLMKSYDQVHSGKLVCGMVSYLNDLPSQRIQ | |
| | PQQVAVWPTMVDINSPESLTEAYKLRAARLVEIAAKNLQKEVIHRK | |
| | SKEVAWNLTSVDLVRASEAHCHYVVVKLFSEKLLKIQDKAIQAVLR | |
| | SLCLLYSLYGISQNAGDFLQGSIMTEPQITQVNQRVKELLTLIRSDAV | |
| | ALVDAFDFQDVTLGSVLGRYDGNVYENLFEWAKNSPLNKAEVHES | |
| | YKHLKSLQSKL | |
| 344 | MWGSDRLAGAGGGGAAVTVAFTNARDCFLHLPRRLVAQLHLLQN | PEX1 |
| | QAIEVVWSHQPAFLSWVEGRHFSDQGENVAEINRQVGQKLGLSNG | |
| | GQVFLKPCSHVVSCQQVEVEPLSADDWEILELHAVSLEQHLLDQIRI | |
| | VFPKAIFPVWVDQQTYIFIQIVALIPAASYGRLETDTKLLIQPKTRRA | |
| | KENTFSKADAEYKKLHSYGRDQKGMMKELQTKQLQSNTVGITESN | |
| | ENESEIPVDSSSVASLWTMIGSIFSFQSEKKQETSWGLTEINAFKNMQ | |
| | SKVVPLDNIFRVCKSQPPSIYNASATSVFHKHCAIHVFPWDQEYFDV | |
| | EPSFTVTYGKLVKLLSPKQQQSKTKQNVLSPEKEKQMSEPLDQKKI | |
| | RSDHNEEDEKACVLQVVWNGLEELNNAIKYTKNVEVLHLGKVWIP | |
| | DDLRKRLNIEMHAVVRITPVEVTPKIPRSLKLQPRENLPKDISEEDIK | |
| | TVFYSWLQQSTTTMLPLVISEEEFIKLETKDGLKEFSLSIVHSWEKEK | |
| | DKNIFLLSPNLLQKTTIQVLLDPMVKEEN | |
| | SEEIDFILPFLKLSSLGGVNSLGVSSLEHITHSLLGRPLSRQLMSLVAG | |
| | LRNGALLLTGGKGSGKSTLAKAICKEAFDKLDAHVERVDCKALRG | |
| | KRLENIQKTLEVAFSEAVWMQPSVVLLDDLDLIAGLPAVPEHEHSP | |
| | DAVQSQRLAHALNDMIKEFISMGSLVALIATSQSQQSLHPLLVSAQG | |
| | VHIFQCVQHIQPPNQEQRCEILCNVIKNKLDCDINKFTDLDLQHVAK | |
| | ETGGFVARDFTVLVDRAIHSRLSRQSISTREKLVLTTLDFQKALRGF | |
| | LPASLRSVNLHKPRDLGWDKIGGLHEVRQILMDTIQLPAKYPELFA | |
| | NLPIRQRTGILLYGPPGTGKTLLAGVIARESRMNFISVKGPELLSKYI | |
| | GASEQAVRDIFIRAQAAKPCILFFDEFESIAPRRGHDNTGVTDRVVN | |
| | QLLTQLDGVEGLQGVYVLAATSRPDLIDPALLRPGRLDKCVYCPPP | |
| | DQVSRLEILNVLSDSLPLADDVDLQHVASVTDSFTGADLKALLYNA | |
| | QLEALHGMLLSSGLQDGSSSSDSDLSLSSMVFLNHSSGSDDSAGDG | |
| | ECGLDQSLVSLEMSEILPDESKFNMYRLYFGSSYESELGNGTSSDLS | |
| | SQCLSAPSSMTQDLPGVPGKDQLFSQPPVLRTASQEGCQELTQEQR | |
| | DQLRADISIIKGRYRSQSGEDESMNQPGPIKTRLAISQSHLMTALGHT | |
| | RPSISEDDWKNFAELYESFQNPKRRKNQSGTMFRPGQKVTLA | |
| 345 | MASRKENAKSANRVLRISQLDALELNKALEQLVWSQFTQCFHGFKP | PEX2 |
| | GLLARFEPEVKACLWVFLWRFTIYSKNATVGQSVLNIKYKNDFSPN | |
| | LRYQPPSKNQKIWYAVCTIGGRWLEERCYDLFRNHHLASFGKVKQ | |
| | CVNFVIGLLKLGGLINFLIFLQRGKFATLTERLLGIHSVFCKPQNICEV | |
| | GFEYMNRELLWHGFAEFLIFLLPLINVQKLKAKLSSWCIPLTGAPNS | |
| | DNTLATSGKECALCGEWPTMPHTIGCEHIFCYFCAKSSFLFDVYFTC | |
| | PKCGTEVHSLQPLKSGIEMSEVNAL | |
| 346 | MLRSVWNFLKRHKKKCIFLGTVLGGVYILGKYGQKKIREIQEREAA | PEX3 |
| | EYIAQARRQYHFESNQRTCNMTVLSMLPTLREALMQQLNSESLTAL | |
| | LKNRPSNKLEIWEDLKIISFTRSTVAVYSTCMLVVLLRVQLNIIGGYI | |
| | YLDNAAVGKNGTTILAPPDVQQQYLSSIQHLLGDGLTELITVIKQAV | |
| | QKVLGSVSLKHSLSLLDLEQKLKEIRNLVEQHKSSSWINKDGSKPLL | |
| | CHYMMPDEETPLAVQACGLSPRDITTIKLLNETRDMLESPDFSTVLN | |
| | TCLNRGFSRLLDNMAEFFRPTEQDLQHGNSMNSLSSVSLPLAKIIPIV | |
| | NGQIHSVCSETPSHFVQDLLTMEQVKDFAANVYEAFSTPQQLEK | |
| 347 | MAMRELVEAECGGANPLMKLAGHFTQDKALRQEGLRPGPWPPGA | PEX5 |
| | PASEAASKPLGVASEDELVAEFLQDQNAPLVSRAPQTFKMDDLLAE | |
| | MQQIEQSNFRQAPQRAPGVADLALSENWAQEFLAAGDAVDVTQD | |
| | YNETDWSQEFISEVTDPLSVSPARWAEEYLEQSEEKLWLGEPEGTA | |
| | TDRWYDEYHPEEDLQHTASDFVAKVDDPKLANSEFLKFVRQIGEG | |
| | QVSLESGAGSGRAQAEQWAAEFIQQQGTSDAWVDQFTRPVNTSAL | |
| | DMEFERAKSAIESDVDFWDKLQAELEEMAKRDAEAHPWLSDYDDL | |
| | TSATYDKGYQFEEENPLRDHPQPFEEGLRRLQEGDLPNAVLLFEAA | |
| | VQQDPKHMEAWQYLGTTQAENEQELLAISALRRCLELKPDNQTAL | |
| | MALAVSFTNESLQRQACETLRDWLRYTPAYAHLVTPAEEGAGGAG | |
| | LGPSKRILGSLLSDSLFLEVKELFLAAVRLDPTSIDPDVQCGLGVLFN | |
| | LSGEYDKAVDCFTAALSVRPNDYLLWNKLGATLANGNQSEEAVAA | |
| | YRRALELQPGYIRSRYNLGISCINLGAHREAVEHFLEALNMQRKSRG | |
| | PRGEGGAMSENIWSTLRLALSMLGQSDAYGAADARDLSTLLTMFG | |
| | LPQ | |
| 348 | MALAVLRVLEPFPTETPPLAVLLPPGGPWPAAELGLVLALRPAGESP | PEX6 |
| | AGPALLVAALEGPDAGTEEQGPGPPQLLVSRALLRLLALGSGAWVR | |
| | ARAVRRPPALGWALLGTSLGPGLGPRVGPLLVRRGETLPVPGPRVL | |
| | ETRPALQGLLGPGTRLAVTELRGRARLCPESGDSSRPPPPPVVSSFA | |
| | VSGTVRRLQGVLGGTGDSLGVSRSCLRGLGLFQGEWVWVAQARES | |
| | SNTSQPHLARVQVLEPRWDLSDRLGPGSGPLGEPLADGLALVPATL | |
| | AFNLGCDPLEMGELRIQRYLEGS | |
| | IAPEDKGSCSLLPGPPFARELHIEIVSSPHYSTNGNYDGVLYRHFQIPR | |
| | VVQEGDVLCVPTIGQVEILEGSPEKLPRWREMFFKVKKTVGEAPDG | |
| | PASAYLADTTHTSLYMVGSTLSPVPWLPSEESTLWSSLSPPGLEALV | |
| | SELCAVLKPRLQPGGALLTGTSSVLLRGPPGCGKTTVVAAACSHLG | |
| | LHLLKVPCSSLCAESSGAVETKLQAIFSRARRCRPAVLLLTAVDLLG | |
| | RDRDGLGEDARVMAVLRHLLLNEDPLNSCPPLMVVATTSRAQDLP | |
| | ADVQTAFPHELEVPALSEGQRLSILRALTAHLPLGQEVNLAQLARR | |
| | CAGFVVGDLYALLTHSSRAACTRIKNSGLAGGLTEEDEGELCAAGF | |
| | PLLAEDFGQALEQLQTAHSQAVGAPKIPSVSWHDVGGLQEVKKEIL | |
| | ETIQLPLEHPELLSLGLRRSGLLLHGPPGTGKTLLAKAVATECSLTFL | |
| | SVKGPELINMYVGQSEENVREVFARARAAAPCIIFFDELDSLAPSRG | |
| | RSGDSGGVMDRVVSQLLAELDGLHSTQ | |
| | DVFVIGATNRPDLLDPALLRPGRFDKLVFVGANEDRASQLRVLSAIT | |
| | RKFKLEPSVSLVNVLDCCPPQLTGADLYSLCSDAMTAALKRRVHDL | |
| | EEGLEPGSSALMLTMEDLLQAAARLQPSVSEQELLRYKRIQRKFAA | |
| | C | |
| 349 | MAPAAASPPEVIRAAQKDEYYRGGLRSAAGGALHSLAGARKWLE | PEX10 |
| | WRKEVELLSDVAYFGLTTLAGYQTLGEEYVSIIQVDPSRIHVPSSLR | |
| | RGVLVTLHAVLPYLLDKALLPLEQELQADPDSGRPLQGSLGPGGRG | |
| | CSGARRWMRHHTATLTEQQRRALLRAVFVLRQGLACLQRLHVAW | |
| | FYIHGVFYHLAKRLTGITYLRVRSLPGEDLRARVSYRLLGVISLLHL | |
| | VLSMGLQLYGFRQRQRARKEWRLHRGLSHRRASLEERAVSRNPLC | |
| | TLCLEERRHPTATPCGHLFCWECITAW | |
| | CSSKAECPLCREKFPPQKLIYLRHYR | |
| 350 | MAEHGAHFTAASVADDQPSIFEVVAQDSLMTAVRPALQHVVKVLA | PEX12 |
| | ESNPTHYGFLWRWFDEIFTLLDLLLQQHYLSRTSASFSENFYGLKRI | |
| | VMGDTHKSQRLASAGLPKQQLWKSIMFLVLLPYLKVKLEKLVSSL | |
| | REEDEYSIHPPSSRWKRFYRAFLAAYPFVNMAWEGWFLVQQLRYIL | |
| | GKAQHHSPLLRLAGVQLGRLTVQDIQALEHKPAKASMMQQPARSV | |
| | SEKINSALKKAVGGVALSLSTGLSVGVFFLQFLDWWYSSENQETIKS | |
| | LTALPTPPPPVHLDYNSDSPLLPKMKTVCPLCRKTRVNDTVLATSG | |
| | YVFCYRCVFHYVRSHQACPITGYPTEVQHLIKLYSPEN | |
| 351 | MASQPPPPPKPWETRRIPGAGPGPGPGPTFQSADLGPTLMTRPGQPA | PEX13 |
| | LTRVPPPILPRPSQQTGSSSVNTFRPAYSSFSSGYGAYGNSFYGGYSP | |
| | YSYGYNGLGYNRLRVDDLPPSRFVQQAEESSRGAFQSIESIVHAFAS | |
| | VSMMMDATFSAVYNSFRAVLDVANHFSRLKIHFTKVFSAFALVRTI | |
| | RYLYRRLQRMLGLRRGSENEDLWAESEGTVACLGAEDRAATSAKS | |
| | WPIFLFFAVILGGPYLIWKLLSTHSDEVTDSINWASGEDDHVVARAE | |
| | YDFAAVSEEEISFRAGDMLNLALKEQQPKVRGWLLASLDGQTTGLI | |
| | PANYVKILGKRKGRKTVESSKVSKQQQSFTNPTLTKGATVADSLDE | |
| | QEAAFESVFVETNKVPVAPDSIGKDGEKQDL | |
| 352 | MASSEQAEQPSQPSSTPGSENVLPREPLIATAVKFLQNSRVRQSPLAT | PEX14 |
| | RRAFLKKKGLTDEEIDMAFQQSGTAADEPSSLGPATQVVPVQPPHLI | |
| | SQPYSPAGSRWRDYGALAIIMAGIAFGFHQLYKKYLLPLILGGREDR | |
| | KQLERMEAGLSELSGSVAQTVTQLQTTLASVQELLIQQQQKIQELA | |
| | HELAAAKATTSTNWILESQNINELKSEINSLKGLLLNRRQFPPSPSAP | |
| | KIPSWQIPVKSPSPSSPAAVNHHSSSDISPVSNESTSSSPGKEGHSPEG | |
| | STVTYHLLGPQEEGEGVVDVKGQVRMEVQGEEEKREDKEDEEDEE | |
| | DDDVSHVDEEDCLGVQREDRRGGDGQINEQVEKLRRPEGASNESE | |
| | RD | |
| 353 | MEKLRLLGLRYQEYVTRHPAATAQLETAVRGFSYLLAGRFADSHE | PEX16 |
| | LSELVYSASNLLVLLNDGILRKELRKKLPVSLSQQKLLTWLSVLECV | |
| | EVFMEMGAAKVWGEVGRWLVIALVQLAKAVLRMLLLLWFKAGL | |
| | QTSPPIVPLDRETQAQPPDGDHSPGNHEQSYVGKRSNRVVRTLQNT | |
| | PSLHSRHWGAPQQREGRQQQHHEELSATPTPLGLQETIAEFLYIARP | |
| | LLHLLSLGLWGQRSWKPWLLAGVVDVTSLSLLSDRKGLTRRERRE | |
| | LRRRTILLLYYLLRSPFYDRFSEARIL | |
| | FLLQLLADHVPGVGLVTRPLMDYLPTWQKIYFYSWG | |
| 354 | MAAAEEGCSVGAEADRELEELLESALDDFDKAKPSPAPPSTTTAPD | PEX19 |
| | ASGPQKRSPGDTAKDALFASQEKFFQELFDSELASQATAEFEKAMK | |
| | ELAEEEPHLVEQFQKLSEAAGRVGSDMTSQQEFTSCLKETLSGLAK | |
| | NATDLQNSSMSEEELTKAMEGLGMDEGDGEGNILPIMQSIMQNLLS | |
| | KDVLYPSLKEITEKYPEWLQSHRESLPPEQFEKYQEQHSVMCKICEQ | |
| | FEAETPTDSETTQKARFEMVLDLMQQLQDLGHPPKELAGEMPPGLN | |
| | FDLDALNLSGPPGASGEQCLIM | |
| 355 | MKSDSSTSAAPLRGLGGPLRSSEPVRAVPARAPAVDLLEEAADLLV | PEX26 |
| | VHLDFRAALETCERAWQSLANHAVAEEPAGTSLEVKCSLCVVGIQ | |
| | ALAEMDRWQEVLSWVLQYYQVPEKLPPKVLELCILLYSKMQEPGA | |
| | VLDVVGAWLQDPANQNLPEYGALAEFHVQRVLLPLGCLSEAEELV | |
| | VGSAAFGEERRLDVLQAIHTARQQQKQEHSGSEEAQKPNLEGSVSH | |
| | KFLSLPMLVRQLWDSAVSHFFSLPFKKSLLAALILCLLVVRFDPASP | |
| | SSLHFLYKLAQLFRWIRKAAFSRLYQ | |
| | LRIRD | |
| 356 | MALQGISVVELSGLAPGPFCAMVLADFGARVVRVDRPGSRYDVSR | AMACR |
| | LGRGKRSLVLDLKQPRGAAVLRRLCKRSDVLLEPFRRGVMEKLQL | |
| | GPEILQRENPRLIYARLSGFGQSGSFCRLAGHDINYLALSGVLSKIGR | |
| | SGENPYAPLNLLADFAGGGLMCALGIIMALFDRTRTGKGQVIDANM | |
| | VEGTAYLSSFLWKTQKLSLWEAPRGQNMLDGGAPFYTTYRTADGE | |
| | FMAVGAIEPQFYELLIKGLGLKSDELPNQMSMDDWPEMKKKFADV | |
| | FAEKTKAEWCQIFDGTDACVTPVLTFEEVVHHDHNKERGSFITSEE | |
| | QDVSPRPAPLLLNTPAIPSFKRDPFIGEHTEEILEEFGFSREEIYQLNSD | |
| | KIIESNKVKASL | |
| 357 | MAQTPAFDKPKVELHVHLDGSIKPETILYYGRRRGIALPANTAEGLL | ADA |
| | NVIGMDKPLTLPDFLAKFDYYMPAIAGCREAIKRIAYEFVEMKAKE | |
| | GVVYVEVRYSPHLLANSKVEPIPWNQAEGDLTPDEVVALVGQGLQ | |
| | EGERDFGVKARSILCCMRHQPNWSPKVVELCKKYQQQTVVAIDLA | |
| | GDETIPGSSLLPGHVQAYQEAVKSGIHRTVHAGEVGSAEVVKEAVD | |
| | ILKTERLGHGYHTLEDQALYNRLRQENMHFEICPWSSYLTGAWKPD | |
| | TEHAVIRLKNDQANYSLNTDDPLIF | |
| | KSTLDTDYQMTKRDMGFTEEEFKRLNINAAKSSFLPEDEKRELLDL | |
| | LYKAYGMPPSASAGQNL | |
| 358 | MAAGGDHGSPDSYRSPLASRYASPEMCFVFSDRYKFRTWRQLWL | ADSL |
| | WLAEAEQTLGLPITDEQIQEMKSNLENIDFKMAAEEEKRLRHDVMA | |
| | HVHTFGHCCPKAAGIIHLGATSCYVGDNTDLIILRNALDLLLPKLAR | |
| | VISRLADFAKERASLPTLGFTHFQPAQLTTVGKRCCLWIQDLCMDL | |
| | QNLKRVRDDLRFRGVKGTTGTQASFLQLFEGDDHKVEQLDKMVTE | |
| | KAGFKRAFIITGQTYTRKVDIEVLSVLASLGASVHKICTDIRLLANLK | |
| | EMEEPFEKQQIGSSAMPYKRNPMRSERCCSLARHLMTLVMDPLQT | |
| | ASVQWFERTLDDSANRRICLAEAFLTADTILNTLQNISEGLVVYPKV | |
| | IERRIRQELPFMATENIIMAMVKAGGSRQDCHEKIRVLSQQAASVVK | |
| | QEGGDNDLIERIQVDAYFSPIHSQLDHLLDPSSFTGRASQQVQRFLEE | |
| | EVYPLLKPYESVMKVKAELCL | |
| 359 | MNVRIFYSVSQSPHSLLSLLFYCAILESRISATMPLFKLPAEEKQIDD | AMPD1 |
| | AMRNFAEKVFASEVKDEGGRQEISPFDVDEICPISHHEMQAHIFHLE | |
| | TLSTSTEARRKKRFQGRKTVNLSIPLSETSSTKLSHIDEYISSSPTYQT | |
| | VPDFQRVQITGDYASGVTVEDFEIVCKGLYRALCIREKYMQKSFQR | |
| | FPKTPSKYLRNIDGEAWVANESFYPVFTPPVKKGEDPFRTDNLPENL | |
| | GYHLKMKDGVVYVYPNEAAVSKDEPKPLPYPNLDTFLDDMNFLLA | |
| | LIAQGPVKTYTHRRLKFLSSKFQVHQMLNEMDELKELKNNPHRDF | |
| | YNCRKVDTHIHAAACMNQKHLLRFIKKSYQIDADRVVYSTKEKNL | |
| | TLKELFAKLKMHPYDLTVDSLDVHAGRQTFQRFDKFNDKYNPVGA | |
| | SELRDLYLKTDNYINGEYFATIIKEVGADLVEAKYQHAEPRLSIYGR | |
| | SPDEWSKLSSWFVCNRIHCPNMTWMIQVPRIYDVFRSKNFLPHFGK | |
| | MLENIFMPVFEATINPQADPELSVFLKHIT | |
| | GFDSVDDESKHSGHMFSSKSPKPQEWTLEKNPSYTYYAYYMYANI | |
| | MVLNSLRKERGMNTFLFRPHCGEAGALTHLMTAFMIADDISHGLNL | |
| | KKSPVLQYLFFLAQIPIAMSPLSNNSLFLEYAKNPFLDFLQKGLMISL | |
| | STDDPMQFHFTKEPLMEEYAIAAQVFKLSTCDMCEVARNSVLQCGI | |
| | SHEEKVKFLGDNYLEEGPAGNDIRRTNVAQIRMAYRYETWCYELN | |
| | LIAEGLKSTE | |
| 360 | MATEGMILTNHDHQIRVGVLTVSDSCFRNLAEDRSGINLKDLVQDP | GPHN |
| | SLLGGTISAYKIVPDEIEEIKETLIDWCDEKELNLILTTGGTGFAPRDV | |
| | TPEATKEVIEREAPGMALAMLMGSLNVTPLGMLSRPVCGIRGKTLII | |
| | NLPGSKKGSQECFQFILPALPHAIDLLRDAIVKVKEVHDELEDLPSPP | |
| | PPLSPPPTTSPHKQTEDKGVQCEEEEEEKKDSGVASTEDSSSSHITAA | |
| | AIAAKIPDSIISRGVQVLPRDTASLSTTPSESPRAQATSRLSTASCPTP | |
| | KVQSRCSSKENILRASHSAVDITKVARRHRMSPFPLTSMDKAFITVL | |
| | EMTPVLGTEIINYRDGMGRVLAQDVYAKDNLPPFPASVKDGYAVR | |
| | AADGPGDRFIIGESQAGEQPTQTVMPGQVMRVTTGAPIPCGADAVV | |
| | QVEDTELIRESDDGTEELEVRILVQARPGQDIRPIGHDIKRGECVLAK | |
| | GTHMGPS | |
| | EIGLLATVGVTEVEVNKFPVVAVMSTGNELLNPEDDLLPGKIRDSN | |
| | RSTLLATIQEHGYPTINLGIVGDNPDDLLNALNEGISRADVIITSGGVS | |
| | MGEKDYLKQVLDIDLHAQIHFGRVFMKPGLPTTFATLDIDGVRKIIF | |
| | ALPGNPVSAVVTCNLFVVPALRKMQGILDPRPTIIKARLSCDVKLDP | |
| | RPEYHRCILTWHHQEPLPWAQSTGNQMSSRLMSMRSANGLLMLPP | |
| | KTEQYVELHKGEVVDVMVIGRL | |
| 361 | MAGAAAESGRELWTFAGSRDPSAPRLAYGYGPGSLRELRAREFSRL | MOCOS |
| | AGTVYLDHAGATLFSQSQLESFTSDLMENTYGNPHSQNISSKLTHD | |
| | TVEQVRYRILAHFHTTAEDYTVIFTAGSTAALKLVAEAFPWVSQGP | |
| | ESSGSRFCYLTDSHTSVVGMRNVTMAINVISTPVRPEDLWSAEERSA | |
| | SASNPDCQLPHLFCYPAQSNFSGVRYPLSWIEEVKSGRLHPVSTPGK | |
| | WFVLLDAASYVSTSPLDLSAHQADFVPISFYKIFGFPTGLGALLVHN | |
| | RAAPLLRKTYFGGGTASAYLAGEDFYIPRQSVAQRFEDGTISFLDVI | |
| | ALKHGFDTLERLTGGMENIKQHTFTLAQYTYVALSSLQYPNGAPVV | |
| | RIYSDSEFSSPEVQGPIINFNVLDDKGNIIGYSQVDKMASLYNIHLRT | |
| | GCFCNTGACQRHLGISNEMVRKHFQAGHVCGDNMDLIDGQPTGSV | |
| | RISFGYMSTLDDVQAFLRFIIDTRLHSSGDWPVPQAHADTGETGAPS | |
| | ADSQADVIPAVMGRRSLSPQEDALTGSRVWNNSSTVNAVPVAPPV | |
| | CDVARTQPTPSEKAAGVLEGALGPHVVTNLYLYPIKSCAAFEVTRW | |
| | PVGNQGLLYDRSWMVVNHNGVCLSQKQEPRLCLIQPFIDLRQRIMV | |
| | IKAKGMEPIEVPLEENSERTQIRQSRVCADRVSTYDCGEKISSWLSTF | |
| | FGRPCHLIKQSSNSQRNAKKKHGKDQLPGTMATLSLVNEAQYLLIN | |
| | TSSILELHRQLNTSDENGKEELFSLKDLSLRFRANIIINGKRAFEEEK | |
| | WDEISIGSLRFQVLGPCHRCQMICIDQQTGQRNQHVFQKLSESRETK | |
| | VNFGMYLMHASLDLSSPCFLSVGSQVLPVLKENVEGHDLPASEKHQ | |
| | DVTS | |
| 362 | MAARPLSRMLRRLLRSSARSCSSGAPVTQPCPGESARAASEEVSRRR | MOCS1 |
| | QFLREHAAPFSAFLTDSFGRQHSYLRISLTEKCNLRCQYCMPEEGVP | |
| | LTPKANLLTTEEILTLARLFVKEGIDKIRLTGGEPLIRPDVVDIVAQLQ | |
| | RLEGLRTIGVTTNGINLARLLPQLQKAGLSAINISLDTLVPAKFEFIVR | |
| | RKGFHKVMEGIHKAIELGYNPVKVNCVVMRGLNEDELLDFAALTE | |
| | GLP | |
| | LDVRFIEYMPFDGNKWNFKKMVSYKEMLDTVRQQWPELEKVPEEE | |
| | SSTAKAFKIPGFQGQISFITSMSEHFCGTCNRLRITADGNLKVCLFGN | |
| | SEVSLRDHLRAGASEQELLRIIGAAVGRKKRQHAGMFSISQMKNRP | |
| | MILIELFLMFPNSPPANPSIFSWDPLHVQGLRPRMSFSSQVATLWKG | |
| | CRVPQTPPLAQQRLGSGSFQRHYTSRADSDANSKCLSPGSWASAAP | |
| | SGPQLTSEQLTHVDSEGRAAMVDVGRKPDTERVAVASAVVLLGPV | |
| | AFKLVQQNQLKKGDALVVAQLAG | |
| | VQAAKVTSQLIPLCHHVALSHIQVQLELDSTRHAVKIQASCRARGPT | |
| | GVEMEALTSAAVAALTLYDMCKAVSRDIVLEEIKLISKTGGQRGDF | |
| | HRA | |
| 363 | MENGYTYEDYKNTAEWLLSHTKHRPQVAIICGSGLGGLTDKLTQA | PNP |
| | QIFDYGEIPNFPRSTVPGHAGRLVFGFLNGRACVMMQGRFHMYEG | |
| | YPLWKVTFPVRVFHLLGVDTLVVTNAAGGLNPKFEVGDIMLIRDHI | |
| | NLPGFSGQNPLRGPNDERFGDRFPAMSDAYDRTMRQRALSTWKQM | |
| | GEQRELQEGTYVMVAGPSFETVAECRVLQKLGADAVGMSTVPEVI | |
| | VARHCGLRVFGFSLITNKVIMDYESLEKANHEEVLAAGKQAAQKLE | |
| | QFVSILMASIPLPDKAS | |
| 364 | MTADKLVFFVNGRKVVEKNADPETTLLAYLRRKLGLSGTKLGCGE | XDH |
| | GGCGACTVMLSKYDRLQNKIVHFSANACLAPICSLHHVAVTTVEGI | |
| | GSTKTRLHPVQERIAKSHGSQCGFCTPGIVMSMYTLLRNQPEPTMEE | |
| | IENAFQGNLCRCTGYRPILQGFRTFARDGGCCGGDGNNPNCCMNQ | |
| | KKDHSVSLSPSLFKPEEFTPLDPTQEPIFPPELLRLKDTPRKQLRFEGE | |
| | RVTWIQASTLKELLDLKAQHPDAKLVVGNTEIGIEMKFKNMLFPMI | |
| | VCPAWIPELNSVEHGPDGISFGAACPLSIVEKTLVDAVAKLPAQKTE | |
| | VFRGVLEQLRWFAGKQVKSVASVGGNIITASPISDLNPVFMASGAK | |
| | LTLVSRGTRRTVQMDHTFFPGYRKTLLSPEEILLSIEIPYSREGEYFSA | |
| | FKQASRREDDIAKVTSGMRVLFKPGTTEVQELALCYGGMANRTISA | |
| | LKTTQRQLSKLWKEELLQDVCAGLAEELHLPPDAPGGMVDFRCTL | |
| | TLSFFFKFYLTVLQKLGQENLEDKCGKLDPTFASATLLFQKDPPADV | |
| | QLFQEVPKGQSEEDMVGRPLPHLAADMQASGEAVYCDDIPRYENE | |
| | LSLRLVTSTRAHAKIKSIDTSEAKKVPGFVCFISADDVPGSNITGICN | |
| | DETVFAKDKVTCVGHIIGAVVADTPEHTQRAAQGVKITYEELPAIITI | |
| | EDAIKNNSFYGPELKIEKGDLKKGFSEADNVVSGEIYIGGQEHFYLE | |
| | THCTIAVPKGEAGEMELFVSTQNTMKTQSFVAKMLGVPANRIVVR | |
| | VKRMGGGFGGKETRSTVVSTAVALAAYKTGRPVRCMLDRDEDML | |
| | ITGGR | |
| | HPFLARYKVGFMKTGTVVALEVDHFSNVGNTQDLSQSIMERALFH | |
| | MDNCYKIPNIRGTGRLCKTNLPSNTAFRGFGGPQGMLIAECWMSEV | |
| | AVTCGMPAEEVRRKNLYKEGDLTHFNQKLEGFTLPRCWEECLASS | |
| | QYHARKSEVDKFNKENCWKKRGLCIIPTKFGISFTVPFLNQAGALLH | |
| | VYTDGSVLLTHGGTEMGQGLHTKMVQVASRALKIPTSKIYISETST | |
| | NTVPNTSPTAASVSADLNGQAVYAACQTILKRLEPYKKKNPSGSWE | |
| | DWVTAAYMDTVSLSATGFYRTPNLGYSFETNSGNPFHYFSYGVAC | |
| | SEVEIDCLTGDHKNLRTDIVMDVGSSLNPAIDIGQVEGAFVQGLGLF | |
| | TLEELHYSPEGSLHTRGPSTYKIPAFGSIPIEFRVSLLRDCPNKKAIYA | |
| | SKAVGEPPLFLAASIFFAIKDAIRAARAQHTGNNVKELFRLDSPATPE | |
| | KIRNACVDKFTTLCVTGVPENCKPWSVRV | |
| 365 | MLLLHRAVVLRLQQACRLKSIPSRICIQACSTNDSFQPQRPSLTFSGD | SUOX |
| | NSSTQGWRVMGTLLGLGAVLAYQDHRCRAAQESTHIYTKEEVSSH | |
| | TSPETGIWVTLGSEVFDVTEFVDLHPGGPSKLMLAAGGPLEPFWAL | |
| | YAVHNQSHVRELLAQYKIGELNPEDKVAPTVETSDPYADDPVRHPA | |
| | LKVNSQRPFNAEPPPELLTENYITPNPIFFTRNHLPVPNLDPDTYRLH | |
| | VVGAPGGQSLSLSLDDLHNFPRYEITVTLQCAGNRRSEMTQVKEVK | |
| | GLEWRTGAISTARWAGARLCDVLAQAGHQLCETEAHVCFEGLDSD | |
| | PTGTAYGASIPLARAMDPEAEVLLAYEMNGQPLPRDHGFPVRVVVP | |
| | GVVGARHVKWLGRVSVQPEESYSHWQRRDYKGFSPSVDWETVDF | |
| | DSAPSIQELPVQSAITEPRDGETVESGEVTIKGYAWSGGGRAVIRVD | |
| | VSLDGGLTWQVAKLDGEEQRPRKAWAWRLWQLKAPVPAGQKEL | |
| | NIVCKAVDDGYNVQPDTVAPIWNLRGVLSNAWHRVHVYVSP | |
| 366 | MFHLRTCAAKLRPLTASQTVKTFSQNRPAAARTFQQIRCYSAPVAA | OGDH |
| | EPFLSGTSSNYVEEMYCAWLENPKSVHKSWDIFFRNTNAGAPPGTA | |
| | YQSPLPLSRGSLAAVAHAQSLVEAQPNVDKLVEDHLAVQSLIRAYQ | |
| | IRGHHVAQLDPLGILDADLDSSVPADIISSTDKLGFYGLDESDLDKVF | |
| | HLPTTTFIGGQESALPLREIIRRLEMAYCQHIGVEFMFINDLEQCQWI | |
| | RQKFETPGIMQFTNEEKRTLLARLVRSTRFEEFLQRKWSSEKRFGLE | |
| | GCEVLIPALKTIIDKSSENGVDYVIMGMPHRGRLNVLANVIRKELEQ | |
| | IFCQFDSKLEAADEGSGDVKYHLGMYHRRINRVTDRNITLSLVANP | |
| | SHLEAADPVVMGKTKAEQFYCGDTEGKKVMSILLHGDAAFAGQGI | |
| | VYETFHLSDLPSYTTHGTVHVVVNNQIGFTTDPRMARSSPYPTDVA | |
| | RVVNAPIFHVNSDDPEAVMYVCKVAAEWRSTFHKDVVVDLVCYR | |
| | RNGHNEMDEPMFTQPLMYKQIRKQKPVLQKYAELLVSQGVVNQPE | |
| | YEEEISKYDKICEEAFARSKDEKILHIKHWLDSPWPGFFTLDGQPRS | |
| | MSCPSTGLTEDILTHIGNVASSVPVENFTIHGGLSRILKTRGEMVKNR | |
| | TVDWALAEYMAFGSLLKEGIHIRLSGQDVERGTFSHRHHVLHDQN | |
| | VDKRTCIPMNHLWPNQAPYTVCNSSLSEYGVLGFELGFAMASPNAL | |
| | VLWEAQFGDFHNTAQCIIDQFICPGQAKWVRQNGIVLLLPHGMEG | |
| | MGPEHSSARPERFLQMCNDDPDVLPDLKEANFDINQLYDCNWVVV | |
| | NCSTPGNFFHVLRRQILLPFRKPLIIFTPKSLLRHPEARSSFDEMLPGT | |
| | HFQRVIPEDGPAAQNPENVKRLLFCTGKVYYDLTRERKARDMVGQ | |
| | VAITRIEQLSPFPFDLLLKEVQKYPNAELAWCQEEHKNQGYYDYVK | |
| | PRLRTTISRAKPVWYAGRDPAAAPATGNKKTHLTELQRLLDTAFDL | |
| | DVFKNFS | |
| 367 | MVGYDPKPDGRNNTKFQVAVAGSVSGLVTRALISPFDVIKIRFQLQ | SLC25A19 |
| | HERLSRSDPSAKYHGILQASRQILQEEGPTAFWKGHVPAQILSIGYG | |
| | AVQFLSFEMLTELVHRGSVYDAREFSVHFVCGGLAACMATLTVHP | |
| | VDVLRTRFAAQGEPKVYNTLRHAVGTMYRSEGPQVFYKGLAPTLI | |
| | AIFPYAGLQFSCYSSLKHLYKWAIPAEGKKNENLQNLLCGSGAGVIS | |
| | KTLTYPLDLFKKRLQVGGFEHARAAFGQVRRYKGLMDCAKQVLQ | |
| | KEGALGFFKGLSPSLLKAALSTGFMF | |
| | FSYEFFCNVFHCMNRTASQR | |
| 368 | MASATAAAARRGLGRALPLFWRGYQTERGVYGYRPRKPESREPQG | DHTKD1 |
| | ALERPPVDHGLARLVTVYCEHGHKAAKINPLFTGQALLENVPEIQA | |
| | LVQTLQGPFHTAGLLNMGKEEASLEEVLVYLNQIYCGQISIETSQLQ | |
| | SQDEKDWFAKRFEELQKETFTTEERKHLSKLMLESQEFDHFLATKF | |
| | STVKRYGGEGAESMMGFFHELLKMSAYSGITDVIIGMPHRGRLNLL | |
| | TGLLQFPPELMFRKMRGLSEFPENFSATGDVLSHLTSSVDLYFGAHH | |
| | PLHVTMLPNPSHLEAVNPVAVGK | |
| | TRGRQQSRQDGDYSPDNSAQPGDRVICLQVHGDASFCGQGIVPETF | |
| | TLSNLPHFRIGGSVHLIVNNQLGYTTPAERGRSSLYCSDIGKLVGCAI | |
| | IHVNGDSPEEVVRATRLAFEYQRQFRKDVIIDLLCYRQWGHNELDE | |
| | PFYTNPIMYKIIRARKSIPDTYAEHLIAGGLMTQEEVSEIKSSYYAKL | |
| | NDHLNNMAHYRPPALNLQAHWQGLAQPEAQITTWSTGVPLDLLRF | |
| | VGMKSVEVPRELQMHSHLLKTHVQSRMEKMMDGIKLDWATAEAL | |
| | ALGSLLAQGFNVRLSGQDVGRGT | |
| | FSQRHAIVVCQETDDTYIPLNHMDPNQKGFLEVSNSPLSEEAVLGFE | |
| | YGMSIESPKLLPLWEAQFGDFFNGAQIIFDTFISGGEAKWLLQSGIVI | |
| | LLPHGYDGAGPDHSSCRIERFLQMCDSAEEGVDGDTVNMFVVHPT | |
| | TPAQYFHLLRRQMVRNFRKPLIVASPKMLLRLPAAVSTLQEMAPGT | |
| | TFNPVIGDSSVDPKKVKTLVFCSGKHFYSLVKQRESLGAKKHDFAII | |
| | RVEELCPFPLDSLQQEMSKYKHVKDHIWSQEEPQNMGPWSFVSPRF | |
| | EKQLACKLRLVGRPPLPVPAV | |
| | GIGTVHLHQHEDILAKTFA | |
| 369 | MASALSYVSKFKSFVILFVTPLLLLPLVILMPAKFVRCAYVIILMAIY | SLC13A5 |
| | WCTEVIPLAVTSLMPVLLFPLFQILDSRQVCVQYMKDTNMLFLGGLI | |
| | VAVAVERWNLHKRIALRTLLWVGAKPARLMLGFMGVTALLSMWI | |
| | SNTATTAMMVPIVEAILQQMEATSAATEAGLELVDKGKAKELPGSQ | |
| | VIFEGPTLGQQEDQERKRLCKAMTLCICYAASIGGTATLTGTGPNVV | |
| | LLGQMNELFPDSKDLVNFASWFAFAFPNMLVMLLFAWLWLQFVY | |
| | MRFNFKKSWGCGLESKKNEKAALKVLQEEYRKLGPLSFAEINVLIC | |
| | FFLLVILWFSRDPGFMPGWLTVAWVEGETKYVSDATVAIFVATLLFI | |
| | VPSQKPKFNFRSQTEEERKTPFYPPPLLDWKVTQEKVPWGIVLLLGG | |
| | GFALAKGSEASGLSVWMGKQMEPLHAVPPAAITLILSLLVAVFTEC | |
| | TSNVATTTLFLPIFASMSRSIGLNPLYIMLPCTLSASFAFMLPVATPPN | |
| | AIVFTYGHLKVADMVKTGVIMNIIGVFCVFLAVNTWGRAIFDLDHF | |
| | PDWANVTHIET | |
| 370 | MYRALRLLARSRPLVRAPAAALASAPGLGGAAVPSFWPPNAARMA | FH |
| | SQNSFRIEYDTFGELKVPNDKYYGAQTVRSTMNFKIGGVTERMPTP | |
| | VIKAFGILKRAAAEVNQDYGLDPKIANAIMKAADEVAEGKLNDHFP | |
| | LVVWQTGSGTQTNMNVNEVISNRAIEMLGGELGSKIPVHPNDHVN | |
| | KSQSSNDTFPTAMHIAAAIEVHEVLLPGLQKLHDALDAKSKEFAQII | |
| | KIGRTHTQDAVPLTLGQEFSGYVQQVKYAMTRIKAAMPRIYELAAG | |
| | GTAVGTGLNTRIGFAEKVAAKVAALTGLPFVTAPNKFEALAAHDA | |
| | LVELSGAMNTTACSLMKIANDIRFLGSGPRSGLGELILPENEPGSSIM | |
| | PGKVNPTQCEAMTMVAAQVMGNHVAVTVGGSNGHFELNVFKPM | |
| | MIKNVLHSARLLGDASVSFTENCVVGIQANTERINKLMNESLMLVT | |
| | ALNPHIGYDKAAKIAKTAHKNGSTLKETAIELGYLTAEQFDEWVKP | |
| | KDMLGPK | |
| 371 | MWRVCARRAQNVAPWAGLEARWTALQEVPGTPRVTSRSGPAPAR | DLAT |
| | RNSVTTGYGGVRALCGWTPSSGATPRNRLLLQLLGSPGRRYYSLPP | |
| | HQKVPLPSLSPTMQAGTIARWEKKEGDKINEGDLIAEVETDKATVG | |
| | FESLEECYMAKILVAEGTRDVPIGAIICITVGKPEDIEAFKNYTLDSSA | |
| | APTPQAAPAPTPAATASPPTPSAQAPGSSYPPHMQVLLPALSPTMTM | |
| | GTVQRWEKKVGEKLSEGDLLAEIETDKATIGFEVQEEGYLAKILVPE | |
| | GTRDVPLGTPLCIIVEKEADISAFADYRPTEVTDLKPQVPPPTPPPVA | |
| | AVPPTPQPLAPTPSAPCPATPAGPKGRVFVSPLAKKLAVEKGIDLTQ | |
| | VKGTGPDGRITKKDIDSFVPSKVAPAPAAVVPPTGPGMAPVPTGVFT | |
| | DIPISNIRRVIAQRLMQSKQTIPHYYLSIDVNMGEVLLVRKELNKILE | |
| | GRSKISVNDFIIKASALACLKVPEANSSWMDTVIRQNHVVDVSVAV | |
| | STPAGLITPIVFNAHIKGVETIANDVVSLATKAREGKLQPHEFQGGTF | |
| | TISNLGMFGIKNFSAIINPPQACILAIGASEDKLVPADNEKGFDVASM | |
| | MSVTLSCDHRVVDGAVGAQWLAEFRKYLEKPITMLL | |
| 372 | MAGALVRKAADYVRSKDFRDYLMSTHFWGPVANWGLPIAAINDM | MPC1 |
| | KKSPEIISGRMTFALCCYSLTFMRFAYKVQPRNWLLFACHATNEVA | |
| | QLIQGGRLIKHEMTKTASA | |
| 373 | MRKMLAAVSRVLSGASQKPASRVLVASRNFANDATFEIKKCDLHR | PDHA1 |
| | LEEGPPVTTVLTREDGLKYYRMMQTVRRMELKADQLYKQKIIRGF | |
| | CHLCDGQEACCVGLEAGINPTDHLITAYRAHGFTFTRGLSVREILAE | |
| | LTGRKGGCAKGKGGSMHMYAKNFYGGNGIVGAQVPLGAGIALAC | |
| | KYNGKDEVCLTLYGDGAANQGQIFEAYNMAALWKLPCIFICENNR | |
| | YGMGTSVERAAASTDYYKRGDFIPGLRVDGMDILCVREATRFAAA | |
| | YCRSGKGPILMELQTYRYHGHSMSDPGVSYRTREEIQEVRSKSDPIM | |
| | LLKDRMVNSNLASVEELKEIDVEVRKEIEDAAQFATADPEPPLEELG | |
| | YHIYSSDPPFEVRGANQWIKFKSVS | |
| 374 | MAAVSGLVRRPLREVSGLLKRRFHWTAPAALQVTVRDAINQGMDE | PDHB |
| | ELERDEKVFLLGEEVAQYDGAYKVSRGLWKKYGDKRIIDTPISEMG | |
| | FAGIAVGAAMAGLRPICEFMTFNFSMQAIDQVINSAAKTYYMSGGL | |
| | QPVPIVFRGPNGASAGVAAQHSQCFAAWYGHCPGLKVVSPWNSED | |
| | AKGLIKSAIRDNNPVVVLENELMYGVPFEFPPEAQSKDFLIPIGKAKI | |
| | ERQGTHITVVSHSRPVGHCLEAAAVLSKEGVECEVINMRTIRPMDM | |
| | ETIEASVMKTNHLVTVEGGWPQFG | |
| | VGAEICARIMEGPAFNFLDAPAVRVTGADVPMPYAKILEDNSIPQVK | |
| | DIIFAIKKTLNI | |
| 375 | MAASWRLGCDPRLLRYLVGFPGRRSVGLVKGALGWSVSRGANWR | PDHX |
| | WFHSTQWLRGDPIKILMPSLSPTMEEGNIVKWLKKEGEAVSAGDAL | |
| | CEIETDKAVVTLDASDDGILAKIVVEEGSKNIRLGSLIGLIVEEGEDW | |
| | KHVEIPKDVGPPPPVSKPSEPRPSPEPQISIPVKKEHIPGTLRFRLSPAA | |
| | RNILEKHSLDASQGTATGPRGIFTKEDALKLVQLKQTGKITESRPTP | |
| | APTATPTAPSPLQATAGPSYPRPVIPPVSTPGQPNAVGTFTEIPASNIR | |
| | RVIAKRLTESKSTVPHAYATADCDLGAVLKVRQDLVKDDIKVSVN | |
| | DFIIKAAAVTLKQMPDVNVSWDGEGPKQLPFIDISVAVATDKGLLTP | |
| | IIKDAAAKGIQEIADSVKALSKKARDGKLLPEEYQGGSFSISNLGMF | |
| | GIDEFTAVINPPQACILAVGRFRPVLKLTEDEEGNAKLQQRQLITVT | |
| | MSSDSRVVDDELATRFLKSFKANLENPIRLA | |
| 376 | MPAPTQLFFPLIRNCELSRIYGTACYCHHKHLCCSSSYIPQSRLRYTP | PDP1 |
| | HPAYATFCRPKENWWQYTQGRRYASTPQKFYLTPPQVNSILKANE | |
| | YSFKVPEFDGKNVSSILGFDSNQLPANAPIEDRRSAATCLQTRGMLL | |
| | GVFDGHAGCACSQAVSERLFYYIAVSLLPHETLLEIENAVESGRALL | |
| | PILQWHKHPNDYFSKEASKLYFNSLRTYWQELIDLNTGESTDIDVKE | |
| | ALINAFKRLDNDISLEAQVGDPNSFLNYLVLRVAFSGATACVAHVD | |
| | GVDLHVANTGDSRAMLGVQEEDGSWSAVTLSNDHNAQNERELER | |
| | LKLEHPKSEAKSVVKQDRLLGLLMPFRAFGDVKFKWSIDLQKRVIE | |
| | SGPDQLNDNEYTKFIPPNYHTPPYLTAEPEVTYHRLRPQDKFLVLAT | |
| | DGLWETMHRQDVVRIVGEYLTGMHHQQPIAVGGYKVTLGQMHGL | |
| | LTERRTKMSSVFEDQNAATHLIRHAVGNNEFGTVDHERLSKMLSLP | |
| | EELARMYRDDITIIVVQFNSHVVGAYQNQE | |
| 377 | MLEKFCNSTFWNSSFLDSPEADLPLCFEQTVLVWIPLGYLWLLAPW | ABCC2 |
| | QLLHVYKSRTKRSSTTKLYLAKQVFVGFLLILAAIELALVLTEDSGQ | |
| | ATVPAVRYTNPSLYLGTWLLVLLIQYSRQWCVQKNSWFLSLFWILS | |
| | ILCGTFQFQTLIRTLLQGDNSNLAYSCLFFISYGFQILILIFSAFSENNE | |
| | SSNNPSSIASFLSSITYSWYDSIILKGYKRPLTLEDVWEVDEEMKTKT | |
| | LVS | |
| | KFETHMKRELQKARRALQRRQEKSSQQNSGARLPGLNKNQSQSQD | |
| | ALVLEDVEKKKKKSGTKKDVPKSWLMKALFKTFYMVLLKSFLLKL | |
| | VNDIFTFVSPQLLKLLISFASDRDTYLWIGYLCAILLFTAALIQSFCLQ | |
| | CYFQLCFKLGVKVRTAIMASVYKKALTLSNLARKEYTVGETVNLM | |
| | SVDAQKLMDVTNFMHMLWSSVLQIVLSIFFLWRELGPSVLAGVGV | |
| | MVLVIPINAILSTKSKTIQVKNMKNKDKRLKIMNEILSGIKILKYFAW | |
| | EPSFRDQVQNLRKKELKNLLAFS | |
| | QLQCVVIFVFQLTPVLVSVVTFSVYVLVDSNNILDAQKAFTSITLFNI | |
| | LRFPLSMLPMMISSMLQASVSTERLEKYLGGDDLDTSAIRHDCNFD | |
| | KAMQFSEASFTWEHDSEATVRDVNLDIMAGQLVAVIGPVGSGKSSL | |
| | ISAMLGEMENVHGHITIKGTTAYVPQQSWIQNGTIKDNILFGTEFNE | |
| | KRYQQVLEACALLPDLEMLPGGDLAEIGEKGINLSGGQKQRISLAR | |
| | ATYQNLDIYLLDDPLSAVDAHVGKHIFNKVLGPNGLLKGKTRLLVT | |
| | HSMHFLPQVDEIVVLGNGTIV | |
| | EKGSYSALLAKKGEFAKNLKTFLRHTGPEEEATVHDGSEEEDDDYG | |
| | LISSVEEIPEDAASITMRRENSFRRTLSRSSRSNGRHLKSLRNSLKTRN | |
| | VNSLKEDEELVKGQKLIKKEFIETGKVKFSIYLEYLQAIGLFSIFFIILA | |
| | FVMNSVAFIGSNLWLSAWTSDSKIFNSTDYPASQRDMRVGVYGAL | |
| | GLAQGIFVFIAHFWSAFGFVHASNILHKQLLNNILRAPMRFFDTTPT | |
| | GRI | |
| | VNRFAGDISTVDDTLPQSLRSWITCFLGIISTLVMICMATPVFTIIVIPL | |
| | GIIYVSVQMFYVSTSRQLRRLDSVTRSPIYSHFSETVSGLPVIRAFEH | |
| | QQRFLKHNEVRIDTNQKCVFSWITSNRWLAIRLELVGNLTVFFSAL | |
| | MMVIYRDTLSGDTVGFVLSNALNITQTLNWLVRMTSEIETNIVAVE | |
| | RITEYTKVENEAPWVTDKRPPPDWPSKGKIQFNNYQVRYRPELDLV | |
| | LRGI | |
| | TCDIGSMEKIGVVGRTGAGKSSLTNCLFRILEAAGGQIIIDGVDIASIG | |
| | LHDLREKLTIIPQDPILFSGSLRMNLDPFNNYSDEEIWKALELAHLKS | |
| | FVASLQLGLSHEVTEAGGNLSIGQRQLLCLGRALLRKSKILVLDEAT | |
| | AAVDLETDNLIQTTIQNEFAHCTVITIAHRLHTIMDSDKVMVLDNGK | |
| | IIECGSPEELLQIPGPFYFMAKEAGIENVNSTKF | |
| 378 | MDQNQHLNKTAEAQPSENKKTRYCNGLKMFLAALSLSFIAKTLGAI | SLCO1B1 |
| | IMKSSIIHIERRFEISSSLVGFIDGSFEIGNLLVIVFVSYFGSKLHRPKLI | |
| | GIGCFIMGIGGVLTALPHFFMGYYRYSKETNINSSENSTSTLSTCLIN | |
| | QILSLNRASPEIVGKGCLKESGSYMWIYVFMGNMLRGIGETPIVPLG | |
| | LSYIDDFAKEGHSSLYLGILNAIAMIGPIIGFTLGSLFSKMYVDIGYV | |
| | DLSTIRITPTDSRWVGAWWLNFLVSGLFSHSSIPFFFLPQTPNKPQKE | |
| | RKASLSLHVLETNDEKDQTANLTNQGKNITKNVTGFFQSFKSILTNP | |
| | LYVMFVLLTLLQVSSYIGAFTYVFKYVEQQYGQPSSKANILLGVITIP | |
| | IFASGMFLGGYIIKKFKLNTVGIAKFSCFTAVMSLSFYLLYFFILCEN | |
| | KSVAGLTMTYDGNNPVTSHRDVPLSYCNSDCNCDESQWEPVCGNN | |
| | GITYISPCLAGCKSSSGNKKPIVFYNCSCLEVTGLQNRNYSAHLGEC | |
| | PRDDACTRKFYFFVAIQVLNLFFSALGGTSHVMLIVKIVQPELKSLA | |
| | LGFHSMVIRALGGILAPIYFGALIDTTCIKWSTNNCGTRGSCRTYNST | |
| | SFSRVYLGLSSMLRVSSLVLYIILIYAMKKKYQEKDINASENGSVMD | |
| | EANLESLNKNKHFVPSAGADSETHC | |
| 379 | MDQHQHLNKTAESASSEKKKTRRCNGFKMFLAALSFSYIAKALGGI | SLCO1B3 |
| | IMKISITQIERRFDISSSLAGLIDGSFEIGNLLVIVFVSYFGSKLHRPKLI | |
| | GIGCLLMGTGSILTSLPHFFMGYYRYSKETHINPSENSTSSLSTCLINQ | |
| | TLSFNGTSPEIVEKDCVKESGSHMWIYVFMGNMLRGIGETPIVPLGIS | |
| | YIDDFAKEGHSSLYLGSLNAIGMIGPVIGFALGSLFAKMYVDIGYV | |
| | DLSTIRITPKDSRWVGAWWLGFLVSGLFSHSSIPFFFLPKNPNKPQKE | |
| | RKISLSLHVLKTNDDRNQTANLTNQGKNVTKNVTGFFQSLKSILTNP | |
| | LYVIFLLLTLLQVSSFIGSFTYVFKYMEQQYGQSASHANFLLGIITIPT | |
| | VATGMFLGGFIIKKFKLSLVGIAKFSFLTSMISFLFQLLYFPLICESKS | |
| | VAGLTLTYDGNNSVASHVDVPLSYCNSECNCDESQWEPVCGNNGI | |
| | TYLSPCLAGCKSSSGIKKHTVFYNCSCVEVTGLQNRNYSAHLGECP | |
| | RDNTCTRKFFIYVAIQVINSLFSATGGTTFILLTVKIVQPELKALAMG | |
| | FQSMVIRTLGGILAPIYFGALIDKTCMKWSTNSCGAQGACRIYNSVF | |
| | FGRVYLGLSIALRFPALVLYIVFIFAMKKKFQGKDTKASDNERKVM | |
| | DEANLEFLNNGEHFVPSAGTDSKTCNLDMQDNAAAN | |
| 380 | MGEPGQSPSPRSSHGSPPTLSTLTLLLLLCGHAHSQCKILRCNAEYVS | HFE2 |
| | STLSLRGGGSSGALRGGGGGGRGGGVGSGGLCRALRSYALCTRRT | |
| | ARTCRGDLAFHSAVHGIEDLMIQHNCSRQGPTAPPPPRGPALPGAGS | |
| | GLPAPDPCDYEGRFSRLHGRPPGFLHCASFGDPHVRSFHHHFHTCR | |
| | VQGAWPLLDNDFLFVQATSSPMALGANATATRKLTIIFKNMQECID | |
| | QKVYQAEVDNLPVAFEDGSINGGDRPGGSSLSIQTANPGNHVEIQA | |
| | AYIGTTIIIRQTAGQLSFSIKVAEDVAMAFSAEQDLQLCVGGCPPSQR | |
| | LSRSERNRRGAITIDTARRLCKEGLPVEDAYFHSCVFDVLISGDPNFT | |
| | VAAQAALEDARAFLPDLEKLHLFPSDAGVPLSSATLLAPLLSGLFVL | |
| | WLCIQ | |
| 381 | MHQRHPRARCPPLCVAGILACGFLLGCWGPSHFQQSCLQALEPQAV | ADAMTS13 |
| | SSYLSPGAPLKGRPPSPGFQRQRQRQRRAAGGILHLELLVAVGPDVF | |
| | QAHQEDTERYVLTNLNIGAELLRDPSLGAQFRVHLVKMVILTEPEG | |
| | APNITANLTSSLLSVCGWSQTINPEDDTDPGHADLVLYITRFDLELPD | |
| | GNRQVRGVTQLGGACSPTWSCLITEDTGFDLGVTIAHEIGHSFGLEH | |
| | DGAPGSGCGPSGHVMASDGAAPRAGLAWSPCSRRQLLSLLSAGRA | |
| | RCVWDPPRPQPGSAGHPPDAQPGLYYSANEQCRVAFGPKAVACTF | |
| | AREHLDMCQALSCHTDPLDQSSCSRLLVPLLDGTECGVEKWCSKG | |
| | RCRSLVELTPIAAVHGRWSSWGPRSPCSRSCGGGVVTRRRQCNNPR | |
| | PAFGGRACVGADLQAEMCNTQACEKTQLEFMSQQCARTDGQPLRS | |
| | SPGGASFYHWGAAVPHSQGDALCRHMCRAIGESFIMKRGDSFLDG | |
| | TRCMPSGPREDGTLSLCVSGSCRTFGCDGRMDSQQVWDRCQVCGG | |
| | DNSTCSPRKGSFTAGRAREYVTFLTVTPNLTSVYIANHRPLFTHLAV | |
| | RIGGRYVVAGKMSISPNTTYPSLLEDGRVEYRVALTEDRLPRLEEIRI | |
| | WGPLQEDADIQVYRRYGEEYGNLTRPDITFTYFQPKPRQAWVWAA | |
| | VRGPCSVSCGAGLRWVNYSCLDQARKELVETVQCQGSQQPPAWPE | |
| | ACVLEPCPPYWAVGDFGPCSASCGGGLRERPVRCVEAQGSLLKTLP | |
| | PARCRAGAQQPAVALETCNPQPCPARWEVSEPSSCTSAGGAGLALE | |
| | NETCVPGADGLEAPVTEGPGSVDEKLPAPEPCVGMSCPPGWGHLD | |
| | ATSAGEKAPSPWGSIRTGAQAAHVWTPAAGSCSVSCGRGLMELRF | |
| | LCMDSALRVPVQEELCGLASKPGSRREVCQAVPCPARWQYKLAAC | |
| | SVSCGRGVVRRILYCARAHGEDDGEEILLDTQCQGLPRPEPQEACSL | |
| | EPCPPRWKVMSLGPCSASCGLGTARRSVACVQLDQGQDVEVDEAA | |
| | CAALVRPEASVPCLIADCTYRWHVGTWMECSVSCGDGIQRRRDTC | |
| | LGPQAQAPVPADFCQHLPKPVTVRGCWAGPCVGQGTPSLVPHEEA | |
| | AAPGRTTATPAGASLEWSQARGLLFSPAPQPRRLLPGPQENSVQSSA | |
| | CGRQHLEPTGTIDMRGPGQADCAVAIGRPLGEVVTLRVLESSLNCS | |
| | AGDMLLLWGRLTWRKMCRKLLDMTFSSKTNTLVVRQRCGRPGGG | |
| | VLLRYGSQLAPETFYRECDMQLFGPWGEIVSPSLSPATSNAGGCRLF | |
| | INVAPHARIAIHALATNMGAGTEGANASYILIRDTHSLRTTAFHGQQ | |
| | VLYWESESSQAEMEFSEGFLKAQASLRGQYWTLQSWVPEMQDPQS | |
| | WKGKEGT | |
| 382 | MSRPLSDQEKRKQISVRGLAGVENVTELKKNFNRHLHFTLVKDRN | PYGM |
| | VATPRDYYFALAHTVRDHLVGRWIRTQQHYYEKDPKRIYYLSLEFY | |
| | MGRTLQNTMVNLALENACDEATYQLGLDMEELEEIEEDAGLGNGG | |
| | LGRLAACFLDSMATLGLAAYGYGIRYEFGIFNQKISGGWQMEEAD | |
| | DWLRYGNPWEKARPEFTLPVHFYGHVEHTSQGAKWVDTQVVLAM | |
| | PYDTPVPGYRNNVVNTMRLWSAKAPNDFNLKDFNVGGYIQAVLD | |
| | RNLAENISRVLYPNDNFFEGKELRLKQEYFVVAATLQDIIRRFKSSK | |
| | FGCRDPVRTNFDAFPDKVAIQLNDTHPSLAIPELMRILVDLERM | |
| | DWDKAWDVTVRTCAYTNHTVLPEALERWPVHLLETLLPRHLQIIYE | |
| | INQRFLNRVAAAFPGDVDRLRRMSLVEEGAVKRINMAHLCIAGSHA | |
| | VNGVARIHSEILKKTIFKDFYELEPHKFQNKTNGITPRRWLVLCNPG | |
| | LAEVIAERIGEDFISDLDQLRKLLSFVDDEAFIRDVAKVKQENKLKF | |
| | AAYLEREYKVHINPNSLFDIQVKRIHEYKRQLLNCLHVITLYNRIKR | |
| | EPNKFFVPRTVMIGGKAAPGYHMAKMIIRLVTAIGDVVNHDPAVG | |
| | DRLRVIFLENYRVSLAEKVIPAADLSEQISTAGTEASGTGNMKFMLN | |
| | GALTIGTMDGANVEMAEEAGEENFFIFGMRVEDVDKLDQRGYNAQ | |
| | EYYDRIPELRQVIEQLSSGFFSPKQPDLFKDIVNMLMHHDRFKVFAD | |
| | YEDYIKCQEKVSALYKNPREWTRMVIRNIATSGKFSSDRTIAQYARE | |
| | IWGVEPSRQRLPAPDEAI | |
| 383 | MLSFVDTRTLLLLAVTLCLATCQSLQEETVRKGPAGDRGPRGERGP | COL1A2 |
| | PGPPGRDGEDGPTGPPGPPGPPGPPGLGGNFAAQYDGKGVGLGPGP | |
| | MGLMGPRGPPGAAGAPGPQGFQGPAGEPGEPGQTGPAGARGPAGP | |
| | PGKAGEDGHPGKPGRPGERGVVGPQGARGFPGTPGLPGFKGIRGHN | |
| | GLDGLKGQPGAPGVKGEPGAPGENGTPGQTGARGLPGERGRVGAP | |
| | GPAGARGSDGSVGPVGPAGPIGSAGPPGFPGAPGPKGEIGAVGNAG | |
| | PAGPAGPRGEVGLPGLSGPVGPPGNP | |
| | GANGLTGAKGAAGLPGVAGAPGLPGPRGIPGPVGAAGATGARGLV | |
| | GEPGPAGSKGESGNKGEPGSAGPQGPPGPSGEEGKRGPNGEAGSAG | |
| | PPGPPGLRGSPGSRGLPGADGRAGVMGPPGSRGASGPAGVRGPNGD | |
| | AGRPGEPGLMGPRGLPGSPGNIGPAGKEGPVGLPGIDGRPGPIGPAG | |
| | ARGEPGNIGFPGPKGPTGDPGKNGDKGHAGLAGARGAPGPDGNNG | |
| | AQGPPGPQGVQGGKGEQGPPGPPGFQGLPGPSGPAGEVGKPGERGL | |
| | HGEFGLPGPAGPRGERGPPGESGAA | |
| | GPTGPIGSRGPSGPPGPDGNKGEPGVVGAVGTAGPSGPSGLPGERGA | |
| | AGIPGGKGEKGEPGLRGEIGNPGRDGARGAPGAVGAPGPAGATGD | |
| | RGEAGAAGPAGPAGPRGSPGERGEVGPAGPNGFAGPAGAAGQPGA | |
| | KGERGAKGPKGENGVVGPTGPVGAAGPAGPNGPPGPAGSRGDGGP | |
| | PGMTGFPGAAGRTGPPGPSGISGPPGPPGPAGKEGLRGPRGDQGPV | |
| | GRTGEVGAVGPPGFAGEKGPSGEAGTAGPPGTPGPQGLLGAPGILG | |
| | LPGSRGERGLPGVAGAVGEPGPLGIAGPPGARGPPGAVGSPGVNGA | |
| | PGEAGRDGNPGNDGPPGRDGQPGHKGERGYPGNIGPVGAAGAPGP | |
| | HGPVGPAGKHGNRGETGPSGPVGPAGAVGPRGPSGPQGIRGDKGEP | |
| | GEKGPRGLPGLKGHNGLQGLPGIAGHHGDQGAPGSVGPAGPRGPA | |
| | GPSGPAGKDGRTGHPGTVGPAGIRGPQGHQGPAGPPGPPGPPGPPG | |
| | VSGGGYDFGYDGDFYRADQPRSAPSLRPKDYEVDATLKSLNNQIET | |
| | LLTPEGSRKNPARTCRDLRLSHPEWSSGYYWIDPNQGCTMDAIKVY | |
| | CDFSTGETCIRAQPENIPAKNWYRSSKDKKHVWLGETINAGSQFEY | |
| | NVEGVTSKEMATQLAFMRLLANYASQNITYHCKNSIAYMDEETGN | |
| | LKKAVILQGSNDVELVAEGNSRFTYTVLVDGCSKKTNEWGKTIIEY | |
| | KTNKPSRLPFLDIAPLDIGGADQEFFVDIGPVCFK | |
| 384 | MNNLLCCALVFLDISIKWTTQETFPPKYLHYDEETSHQLLCDKCPPG | TNFRSF11B |
| | TYLKQHCTAKWKTVCAPCPDHYYTDSWHTSDECLYCSPVCKELQY | |
| | VKQECNRTHNRVCECKEGRYLEIEFCLKHRSCPPGFGVVQAGTPER | |
| | NTVCKRCPDGFFSNETSSKAPCRKHTNCSVFGLLLTQKGNATHDNI | |
| | CSGNSESTQKCGIDVTLCEEAFFRFAVPTKFTPNWLSVLVDNLPGTK | |
| | VNAESVERIKRQHSSQEQTFQLLKLWKHQNKDQDIVKKIIQDIDLCE | |
| | NSVQRHIGHANLTFEQLRSLMESLPGKKVGAEDIEKTIKACKPSDQI | |
| | LKLLSLWRIKNGDQDTLKGLMHALKHSKTYHFPKTVTQSLKKTIRF | |
| | LHSFTMYKLYQKLFLEMIGNQVQSVKISCL | |
| 385 | MAQQANVGELLAMLDSPMLGVRDDVTAVFKENLNSDRGPMLVNT | TSC1 |
| | LVDYYLETSSQPALHILTTLQEPHDKHLLDRINEYVGKAATRLSILSL | |
| | LGHVIRLQPSWKHKLSQAPLLPSLLKCLKMDTDVVVLTTGVLVLIT | |
| | MLPMIPQSGKQHLLDFFDIFGRLSSWCLKKPGHVAEVYLVHLHASV | |
| | YALFHRLYGMYPCNFVSFLRSHYSMKENLETFEEVVKPMMEHVRI | |
| | HPELVTGSKDHELDPRRWKRLETHDVVIECAKISLDPTEASYEDGYS | |
| | VSHQISARFPHRSADVTTSPYADT | |
| | QNSYGCATSTPYSTSRLMLLNMPGQLPQTLSSPSTRLITEPPQATLW | |
| | SPSMVCGMTTPPTSPGNVPPDLSHPYSKVFGTTAGGKGTPLGTPATS | |
| | PPPAPLCHSDDYVHISLPQATVTPPRKEERMDSARPCLHRQHHLLND | |
| | RGSEEPPGSKGSVTLSDLPGFLGDLASEEDSIEKDKEEAAISRELSEIT | |
| | TAEAEPVVPRGGFDSPFYRDSLPGSQRKTHSAASSSQGASVNPEPLH | |
| | SSL | |
| | DKLGPDTPKQAFTPIDLPCGSADESPAGDRECQTSLETSIFTPSPCKIP | |
| | PPTRVGFGSGQPPPYDHLFEVALPKTAHHFVIRKTEELLKKAKGNTE | |
| | EDGVPSTSPMEVLDRLIQQGADAHSKELNKLPLPSKSVDWTHFGGS | |
| | PPSDEIRTLRDQLLLLHNQLLYERFKRQQHALRNRRLLRKVIKAAAL | |
| | EEHNAAMKDQLKLQEKDIQMWKVSLQKEQARYNQLQEQRDTMVT | |
| | KLHSQIRQLQHDREEFYNQSQELQTKLEDCRNMIAELRIELKKANN | |
| | KVCHTELLLSQVSQKLSNSESVQQQMEFLNRQLLVLGEVNELYLEQ | |
| | LQNKHSDTTKEVEMMKAAYRKELEKNRSHVLQQTQRLDTSQKRIL | |
| | ELESHLAKKDHLLLEQKKYLEDVKLQARGQLQAAESRYEAQKRIT | |
| | QVFELEILDLYGRLEKDGLLKKLEEEKAEAAEAAEERLDCCNDGCS | |
| | DSMVGHNEEASGHNGETKTPRPSSARGSSGSRGGGGSSSSSSELSTP | |
| | EKPPHQRAGPFSSRWETTMGEASASIPTTVGSLPSSKSFLGMKAREL | |
| | FRNKSESQCDEDGMTSSLSESLKTELGKDLGVEAKIPLNLDGPHPSP | |
| | PTPDSVGQLHIMDYNETHHEHS | |
| 386 | MAKPTSKDSGLKEKFKILLGLGTPRPNPRSAEGKQTEFIITAEILRELS | TSC2 |
| | MECGLNNRIRMIGQICEVAKTKKFEEHAVEALWKAVADLLQPERPL | |
| | EARHAVLALLKAIVQGQGERLGVLRALFFKVIKDYPSNEDLHERLE | |
| | VFKALTDNGRHITYLEEELADFVLQWMDVGLSSEFLLVLVNLVKFN | |
| | SCYLDEYIARMVQMICLLCVRTASSVDIEVSLQVLDAVVCYNCLPA | |
| | ESLPLFIVTLCRTINVKELCEPCWKLMRNLLGTHLGHSAIYNMCHL | |
| | MEDRAYMEDAPLLRGAVFFVGMALWGAHRLYSLRNSPTSVLPSFY | |
| | QAMACPNEVVSYEIVLSITRLIKKYRKELQVVAWDILLNIIERLLQQL | |
| | QTLDSPELRTIVHDLLTTVEELCDQNEFHGSQERYFELVERCADQRP | |
| | ESSLLNLISYRAQSIHPAKDGWIQNLQALMERFFRSESRGAVRIKVL | |
| | DVLSFVLLINRQFYEEELINSVVISQLSHIPEDKDHQVRKLATQLLVD | |
| | LAEGCHTHHFNSLLDIIEKVMARSLSPPPELEERDVAAYSASLEDVK | |
| | TAVLGLLVILQTKLYTLPASHATRVYEMLVSHIQLHYKHSYTLPIAS | |
| | SIRLQAFDFLLLLRADSLHRLGLPNKDGVVRFSPYCVCDYMEPERGS | |
| | E1(KTSGPLSPPTGPPGPAPAGPAVRLGSVPYSLLFRVLLQCLKQESD | |
| | WKVLKLVLGRLPESLRYKVLIFTSPCSVDQLCSALCSMLSGPKTLER | |
| | LRGAPEGFSRTDLHLAVVPVLTALISYHNYL | |
| | DKTKQREMVYCLEQGLIHRCASQCVVALSICSVEMPDIIIKALPVLV | |
| | VKLTHISATASMAVPLLEFLSTLARLPHLYRNFAAEQYASVFAISLP | |
| | YTNPSKFNQYIVCLAHHVIAMWFIRCRLPFRKDFVPFITKGLRSNVL | |
| | LSFDDTPEKDSFRARSTSLNERPKSLRIARPPKQGLNNSPPVKEFKES | |
| | SAAEAFRCRSISVSEHVVRSRIQTSLTSASLGSADENSVAQADDSLK | |
| | NLHL | |
| | ELTETCLDMMARYVFSNFTAVPKRSPVGEFLLAGGRTKTWLVGNK | |
| | LVTVTTSVGTGTRSLLGLDSGELQSGPESSSSPGVHVRQTKEAPAKL | |
| | ESQAGQQVSRGARDRVRSMSGGHGLRVGALDVPASQFLGSATSPG | |
| | PRTAPAAKPEKASAGTRVPVQEKTNLAAYVPLLTQGWAEILVRRPT | |
| | GNTSWLMSLENPLSPFSSDINNMPLQELSNALMAAERFKEHRDTAL | |
| | YKSLSVPAASTAKPPPLPRSNTVASFSSLYQSSCQGQLHRSVSWADS | |
| | AVVMEEGSPGEVPVLVEPPGLEDV | |
| | EAALGMDRRTDAYSRSSSVSSQEEKSLHAEELVGRGIPIERVVSSEG | |
| | GRPSVDLSFQPSQPLSKSSSSPELQTLQDILGDPGDKADVGRLSPEVK | |
| | ARSQSGTLDGESAAWSASGEDSRGQPEGPLPSSSPRSPSGLRPRGYTI | |
| | SDSAPSRRGKRVERDALKSRATASNAEKVPGINPSFVFLQLYHSPFF | |
| | GDESNKPILLPNESQSFERSVQLLDQIPSYDTHKIAVLYVGEGQSNSE | |
| | LA | |
| | ILSNEHGSYRYTEFLTGLGRLIELKDCQPDKVYLGGLDVCGEDGQFT | |
| | YCWHDDIMQAVFHIATLMPTKDVDKHRCDKKRHLGNDFVSIVYND | |
| | SGEDFKLGTIKGQFNFVHVIVTPLDYECNLVSLQCRKDMEGLVDTS | |
| | VAKIVSDRNLPFVARQMALHANMASQVHHSRSNPTDIYPSKWIARL | |
| | RHIKRLRQRICEEAAYSNPSLPLVHPPSHSKAPAQTPAEPTPGYEVG | |
| | QRKRLISSVEDFTEFV | |
| 387 | MAAKSQPNIPKAKSLDGVTNDRTASQGQWGRAWEVDWFSLASVIF | DHCR7 |
| | LLLFAPFIVYYFIMACDQYSCALTGPVVDIVTGHARLSDIWAKTPPIT | |
| | RKAAQLYTLWVTFQVLLYTSLPDFCHKFLPGYVGGIQEGAVTPAGV | |
| | VNKYQINGLQAWLLTHLLWFANAHLLSWFSPTIIFDNWIPLLWCAN | |
| | ILGYAVSTFAMVKGYFFPTSARDCKFTGNFFYNYMMGIEFNPRIGK | |
| | WFDFKLFFNGRPGIVAWTLINLSFAAKQRELHSHVTNAMVLVNVL | |
| | QAIYVIDFFWNETWYLKTIDICHD | |
| | HFGWYLGWGDCVWLPYLYTLQGLYLVYHPVQLSTPHAVGVLLLG | |
| | LVGYYIFRVANHQKDLFRRTDGRCLIWGRKPKVIECSYTSADGQRH | |
| | HSKLLVSGFWGVARHFNYVGDLMGSLAYCLACGGGHLLPYFYIIY | |
| | MAILLTHRCLRDEHRCASKYGRDWERYTAAVPYRLLPGIF | |
| 388 | MSLSNKLTLDKLDVKGKRVVMRVDFNVPMKNNQITNNQRIKAAVP | PGK1 |
| | SIKFCLDNGAKSVVLMSHLGRPDGVPMPDKYSLEPVAVELKSLLGK | |
| | DVLFLKDCVGPEVEKACANPAAGSVILLENLRFHVEEEGKGKDASG | |
| | NKVKAEPAKIEAFRASLSKLGDVYVNDAFGTAHRAHSSMVGVNLP | |
| | QKAGGFLMKKELNYFAKALESPERPFLAILGGAKVADKIQLINNML | |
| | DKVNEMIIGGGMAFTFLKVLNNMEIGTSLFDEEGAKIVKDLMSKAE | |
| | KNGVKITLPVDFVTADKFDENAKTGQATVASGIPAGWMGLDCGPE | |
| | SSKKYAEAVTRAKQIVWNGPVGVFEWEAFARGTKALMDEVV | |
| | KATSRGCITIIGGGDTATCCAKWNTEDKVSHVSTGGGASLELLEGK | |
| | VLPGVDALSNI | |
| 389 | MGTSALWALWLLLALCWAPRESGATGTGRKAKCEPSQFQCTNGR | VLDLR |
| | CITLLWKCDGDEDCVDGSDEKNCVKKTCAESDFVCNNGQCVPSRW | |
| | KCDGDPDCEDGSDESPEQCHMRTCRIHEISCGAHSTQCIPVSWRCD | |
| | GENDCDSGEDEENCGNITCSPDEFTCSSGRCISRNFVCNGQDDCSDG | |
| | SDELDCAPPTCGAHEFQCSTSSCIPISWVCDDDADCSDQSDESLEQC | |
| | GRQPVIHTKCPASEIQCGSGECIHKKWRCDGDPDCKDGSDEVNCPS | |
| | RTCRPDQFECEDGSCIHGSRQCNGI | |
| | RDCVDGSDEVNCKNVNQCLGPGKFKCRSGECIDISKVCNQEQDCR | |
| | DWSDEPLKECHINECLVNNGGCSHICKDLVIGYECDCAAGFELIDRK | |
| | TCGDIDECQNPGICSQICINLKGGYKCECSRGYQMDLATGVCKAVG | |
| | KEPSLIFTNRRDIRKIGLERKEYIQLVEQLRNTVALDADIAAQKLFW | |
| | ADLSQKAIFSASIDDKVGRHVKMIDNVYNPAAIAVDWVYKTIYWT | |
| | DAASKTISVATLDGTKRKFLFNSDLREPASIAVDPLSGFVYWSDWG | |
| | EPAKIEKAGMNGFDRRPLVTADIQ | |
| | WPNGITLDLIKSRLYWLDSKLHMLSSVDLNGQDRRIVLKSLEFLAHP | |
| | LALTIFEDRVYWIDGENEAVYGANKFTGSELATLVNNLNDAQDIIV | |
| | YHELVQPSGKNWCEEDMENGGCEYLCLPAPQINDHSPKYTCSCPSG | |
| | YNVEENGRDCQSTATTVTYSETKDTNTTEISATSGLVPGGINVTTAV | |
| | SEVSVPPKGTSAAWAILPLLLLVMAAVGGYLMWRNWQHKNMKS | |
| | MNFDNPVYLKTTEEDLSIDIGRHSASVGHTYPAISVVSTDDDLA | |
| 390 | MEPSSLELPADTVQRIAAELKCHPTDERVALHLDEEDKLRHFRECFY | KYNU |
| | IPKIQDLPPVDLSLVNKDENAIYFLGNSLGLQPKMVKTYLEEELDKW | |
| | AKIAAYGHEVGKRPWITGDESIVGLMKDIVGANEKEIALMNALTVN | |
| | LHLLMLSFFKPTPKRYKILLEAKAFPSDHYAIESQLQLHGLNIEESMR | |
| | MIKPREGEETLRIEDILEVIEKEGDSIAVILFSGVHFYTGQHFNIPAITK | |
| | AGQAKGCYVGFDLAHAVGNVELYLHDWGVDFACWCSYKYLNAG | |
| | AGGIAGAFIHEKHAHTIKPALVGWFGHELSTRFKMDNKLQLIPGVC | |
| | GFRISNPPILLVCSLHASLEIFKQATMKALRKKSVLLTGYLEYLIKHN | |
| | YGKDKAATKKPVVNIITPSHVEERGCQLTITFSVPNKDVFQELEKRG | |
| | VVCDKRNPNGIRVAPVPLYNSFHDVYKFTNLLTSILDSAETKN | |
| 391 | MFPGCPRLWVLVVLGTSWVGWGSQGTEAAQLRQFYVAAQGISWS | F5 |
| | YRPEPTNSSLNLSVTSFKKIVYREYEPYFKKEKPQSTISGLLGPTLYA | |
| | EVGDIIKVHFKNKADKPLSIHPQGIRYSKLSEGASYLDHTFPAEKMD | |
| | DAVAPGREYTYEWSISEDSGPTHDDPPCLTHIYYSHENLIEDFNSGLI | |
| | GPLLICKKGTLTEGGTQKTFDKQIVLLFAVFDESKSWSQSSSLMYTV | |
| | NGYVNGTMPDITVCAHDHISWHLLGMSSGPELFSIHFNGQVLEQNH | |
| | HKVSAITLVSATSTTANMTVGPEGKWIISSLTPKHLQAGMQAYIDIK | |
| | NCPKKTRNLKKITREQRRHMKRWEYFIAAEEVIWDYAPVIPANMD | |
| | KKYRSQHLDNFSNQIGKHYKKVMYTQYEDESFTKHTVNPNMKED | |
| | GILGPIIRAQVRDTLKIVFKNMASRPYSIYPHGVTFSPYEDEVNSSFTS | |
| | GRNNTMIRAVQPGETYTYKWNILEFDEPTENDAQCLTRPYYSDVDI | |
| | MRDIASGLIGLLLICKSRSLDRRGIQRAA | |
| | DIEQQAVFAVFDENKSWYLEDNINKFCENPDEVKRDDPKFYESNIM | |
| | STINGYVPESITTLGFCFDDTVQWHFCSVGTQNEILTIHFTGHSFIYG | |
| | KRHEDTLTLFPMRGESVTVTMDNVGTWMLTSMNSSPRSKKLRLKF | |
| | RDVKCIPDDDEDSYEIFEPPESTVMATRKMHDRLEPEDEESDADYD | |
| | YQNRLAAALGIRSFRNSSLNQEEEEFNLTALALENGTEFVSSNTDIIV | |
| | GSNYSSPSNISKFTVNNLAEPQKAPSHQQATTAGSPLRHLIGKNSVL | |
| | NSSTAEHSSPYSEDPIEDPLQPDVTGIRLLSLGAGEFKSQEHAKHKGP | |
| | KVERDQAAKHRFSWMKLLAHKVGRHLSQDTGSPSGMRPWEDLPS | |
| | QDTGSPSRMRPWKDPPSDLLLLKQSNSSKILVGRWHLASEKGSYEII | |
| | QDTDEDTAVNNWLISPQNASRAWGESTPLANKPGKQSGHPKFPRV | |
| | RHKSLQVRQDGGKSRLKKSQFLIKTRKKKKEKHTHHAPLSPRTFHP | |
| | LRSEAYNTFSERRLKHSLVLHKSNETSLPT | |
| | DLNQTLPSMDFGWIASLPDHNQNSSNDTGQASCPPGLYQTVPPEEH | |
| | YQTFPIQDPDQMHSTSDPSHRSSSPELSEMLEYDRSHKSFPTDISQMS | |
| | PSSEHEVWQTVISPDLSQVTLSPELSQTNLSPDLSHTTLSPELIQRNLS | |
| | PALGQMPISPDLSHTTLSPDLSHTTLSLDLSQTNLSPELSQTNLSPAL | |
| | GQMPLSPDLSHTTLSLDFSQTNLSPELSHMTLSPELSQTNLSPALGQ | |
| | MP | |
| | ISPDLSHTTLSLDFSQTNLSPELSQTNLSPALGQMPLSPDPSHTTLSLD | |
| | LSQTNLSPELSQTNLSPDLSEMPLFADLSQIPLTPDLDQMTLSPDLGE | |
| | TDLSPNFGQMSLSPDLSQVTLSPDISDTTLLPDLSQISPPPDLDQIFYP | |
| | SESSQSLLLQEFNESFPYPDLGQMPSPSSPTLNDTFLSKEFNPLVIVGL | |
| | SKDGTDYIEIIPKEEVQSSEDDYAEIDYVPYDDPYKTDVRTNINSSRD | |
| | PDNIAAWYLRSNNGNRRNYYIAAEEISWDYSEFVQRETDIEDSDDIP | |
| | EDTTYKKVVFRKYLDSTFTKRDPRGEYEEHLGILGPIIRAEVDDVIQ | |
| | VRFKNLASRPYSLHAHGLSYEKSSEGKTYEDDSPEWFKEDNAVQPN | |
| | SSYTYVWHATERSGPESPGSACRAWAYYSAVNPEKDIHSGLIGPLLI | |
| | CQKGILHKDSNMPMDMREFVLLFMTFDEKKSWYYEKKSRSSWRLT | |
| | SSEMK | |
| | KSHEFHAINGMIYSLPGLKMYEQEWVRLHLLNIGGSQDIHVVHFHG | |
| | QTLLENGNKQHQLGVWPLLPGSFKTLEMKASKPGWWLLNTEVGE | |
| | NQRAGMQTPFLIMDRDCRMPMGLSTGIISDSQIKASEFLGYWEPRL | |
| | ARLNNGGSYNAWSVEKLAAEFASKPWIQVDMQKEVIITGIQTQGAK | |
| | HYLKSCYTTEFYVAYSSNQINWQIFKGNSTRNVMYFNGNSDASTIK | |
| | ENQFDPPIVARYIRISPTRAYNRPTLRLELQGCEVNGCSTPLGMENG | |
| | KIENKQITASSFKKSWWGDYWEPFR | |
| | ARLNAQGRVNAWQAKANNNKQWLEIDLLKIKKITAIITQGCKSLSS | |
| | EMYVKSYTIHYSEQGVEWKPYRLKSSMVDKIFEGNTNTKGHVKNF | |
| | FNPPIISRFIRVIPKTWNQSIALRLELFGCDIY | |
| 392 | MGPTSGPSLLLLLLTHLPLALGSPMYSIITPNILRLESEETMVLEAHD | C3 |
| | AQGDVPVTVTVHDFPGKKLVLSSEKTVLTPATNHMGNVTFTIPANR | |
| | EFKSEKGRNKFVTVQATFGTQVVEKVVLVSLQSGYLFIQTDKTIYTP | |
| | GSTVLYRIFTVNHKLLPVGRTVMVNIENPEGIPVKQDSLSSQNQLGV | |
| | LPLSWDIPELVNMGQWKIRAYYENSPQQVFSTEFEVKEYVLPSFEVI | |
| | VEPTEKFYYIYNEKGLEVTITARFLYGKKVEGTAFVIFGIQDGEQRIS | |
| | LPESLKRIPIEDGSGEVVLSRKVLLDGVQNPRAEDLVGKSLYVSATV | |
| | ILHSGSDMVQAERSGIPIVTSPYQIHFTKTPKYFKPGMPFDLMVFVT | |
| | NPDGSPAYRVPVAVQGEDTVQSLTQGDGVAKLSINTHPSQKPLSITV | |
| | RTKKQELSEAEQATRTMQALPYSTVGNSNNYLHLSVLRTELRPGET | |
| | LNVNFLLRMDRAHEAKIRYYTYLIMNKGRLLKAGRQVREPGQDLV | |
| | VLPLSITTDFIPSFRLVAYYTLIGASGQREVVADSVWVDVKDSCVGS | |
| | LVVKSGQSEDRQPVPGQQMTLKIEGDHGARVVLVAVDKGVFVLNK | |
| | KNKLTQSKIWDVVEKADIGCTPGSGKDYAGVFSDAGLTFTSSSGQQ | |
| | TAQRAELQCPQPAARRRRSVQLTEKRMDKVGKYPKELRKCCEDG | |
| | MRENPMRFSCQRRTRFISLGEACKKVFLDCCNYITELRRQHARASH | |
| | LGLARSNLDEDIIAEENIVSRSEFPESWLWNVEDLKEPPKNGISTKLM | |
| | NIFLKDSITTWEILAVSMSDKKGICVADPFEVTVMQDFFIDLRLPYSV | |
| | VRNEQVEIRAVLYNYRQNQELKVRVELLHNPAFCSLATTKRRHQQ | |
| | TVTIPPKSSLSVPYVIVPLKTGLQEVEVKAAVYHHFISDGVRKSLKV | |
| | VPEGIRMNKTVAVRTLDPERLGREGVQKEDIPPADLSDQVPDTESET | |
| | RILLQGTPVAQMTEDAVDAERLKHLIVTPSGCGEQNMIGMTPTVIA | |
| | VHYLDETEQWEKFGLEKRQGALELIKKGYTQQLAFRQPSSAFAAFV | |
| | KRAPSTWLTA | |
| | YVVKVFSLAVNLIAIDSQVLCGAVKWLILEKQKPDGVFQEDAPVIH | |
| | QEMIGGLRNNNEKDMALTAFVLISLQEAKDICEEQVNSLPGSITKAG | |
| | DFLEANYMNLQRSYTVAIAGYALAQMGRLKGPLLNKFLTTAKDKN | |
| | RWEDPGKQLYNVEATSYALLALLQLKDFDFVPPVVRWLNEQRYYG | |
| | GGYGSTQATFMVFQALAQYQKDAPDHQELNLDVSLQLPSRSSKITH | |
| | RIHWESASLLRSEETKENEGFTVTAEGKGQGTLSVVTMYHAKAKD | |
| | QLTCNKFDLKVTIKPAPETEKRPQDAKNTMILEICTRYRGDQDATM | |
| | SILDISMMTGFAPDTDDLKQLANGVDRYISKYELDKAFSDRNTLIIY | |
| | LDKVSHSEDDCLAFKVHQYFNVELIQPGAVKVYAYYNLEESCTRFY | |
| | HPEKEDGKLNKLCRDELCRCAEENCFIQKSDDKVTLEERLDKACEP | |
| | GVDYVYKTRLVKVQLSNDFDEYIMAIEQTIKSGSDEVQVGQQRTFIS | |
| | PIKCREALKLEEKKHYLMWGLSSDFWGEKPNLSYIIGKDTWVEHWP | |
| | EEDECQDEENQKQCQDLGAFTESMVVFGCPN | |
| 393 | MGPRLSVWLLLLPAALLLHEEHSRAAAKGGCAGSGCGKCDCHGV | COL4A1 |
| | KGQKGERGLPGLQGVIGFPGMQGPEGPQGPPGQKGDTGEPGLPGTK | |
| | GTRGPPGASGYPGNPGLPGIPGQDGPPGPPGIPGCNGTKGERGPLGP | |
| | PGLPGFAGNPGPPGLPGMKGDPGEILGHVPGMLLKGERGFPGIPGTP | |
| | GPPGLPGLQGPVGPPGFTGPPGPPGPPGPPGEKGQMGLSFQGPKGDK | |
| | GDQGVSGPPGVPGQAQVQEKGDFATKGEKGQKGEPGFQGMPGVG | |
| | EKGEPGKPGPRGKPGKDGDKGEKGSPGFPGEPGYPGLIGRQGPQGE | |
| | KGEAGPPGPPGIVIGTGPLGEKGERGYPGTPGPRGEPGPKGFPGLPG | |
| | QPGPPGLPVPGQAGAPGFPGERGEKGDRGFPGTSLPGPSGRDGLPGP | |
| | PGSPGPPGQPGYTNGIVECQPGPPGDQGPPGIPGQPGFIGEIGEKGQK | |
| | GESCLICDIDGYRGPPGPQGPPGEIGFPGQPGAKGDRGLPGRDGVAG | |
| | VPGPQGTPGLIGQPGAKGEPGEFYFDLRLKGDKGDPGFPGQPGMPG | |
| | RAGSPGRDGHPGLPGPKGSPGSVGLKGERGPPGGVGFPGSRGDTGP | |
| | PGPPGYGPAGPIGDKGQAGFPGGPGSPGLPGPKGEPGKIVPLPGPPG | |
| | AEGLPGSPGFPGPQGDRGFPGTPGRPGLPGEKGAVGQPGIGFPGPPG | |
| | PKGVDGLPGDMGPPGTPGRPGFNGLPGNPGVQGQKGEPGVGLPGL | |
| | KGLPGLPGIPGTPGEKGSIGVPGVPGEHGAIGPPGLQGIRGEPGPPGL | |
| | PGSVGSPGVPGIGPPGARGPPGGQGPPGLSGPPGIKGEKGFPGFPGLD | |
| | MPGPKGDKGAQGLPGITGQSGLPGLPGQQGAPGIPGFPGSKGEMGV | |
| | MGTPGQPGSPGPVGAPGLPGEKGDHGFPGSSGPRGDPGLKGDKGD | |
| | VGLPGKPGSMDKVDMGSMKGQKGDQGEKGQIGPIGEKGSRGDPGT | |
| | PGVPGKDGQAGQPGQPGPKGDPGISGTPGAPGLPGPKGSVGGMGLP | |
| | GTPGEKGVPGIPGPQGSPGLPGDKGAKGEKGQAGPPGIGIPGLRGEK | |
| | GDQGIAGFPGSPGEKGEKGSIGIPGMPGSPGLKGSPGSVGYPGSPGLP | |
| | GEKGDKGLPGLDGIPGVKGEAGLPGTPGPTGPAGQKGEPGSDGIPG | |
| | SAGEKGEPGLPGRGFPGFPGAKGDKGSKGEVGFPGLAGSPGIPGSK | |
| | GEQGFMGPPGPQGQPGLPGSPGHATEGPKGDRGPQGQPGLPGLPGP | |
| | MGPPGLPGIDGVKGDKGNPGWPGAPGVPGPKGDPGFQGMPGIGGS | |
| | PGITGSKGDMGPPGVPGFQGPKGLPGLQGIKGDQGDQGVPGAKGLP | |
| | GPPGPPGPYDIIKGEPGLPGPEGPPGLKGLQGLPGPKGQQGVTGLVG | |
| | IPGPPGIPGFDGAPGQKGEMGPAGPTGPRGFPGPPGPDGLPGSMGPP | |
| | GTPSVDHGFLVTRHSQTIDDPQCPSGTKILYHGYSLLYVQGNERAH | |
| | GQDLGTAGSCLRKFSTMPFLFCNINNVCNFASRNDYSYWLSTPEPM | |
| | PMSMAPITGENIRPFISRCAVCEAPAMVMAVHSQTIQIPPCPSGWSSL | |
| | WIGYSFVMHTSAGAEGSGQALASPGSCLEEFRSAPFIECHGRGTCNY | |
| | YANAYSFWLATIERSEMFKKPTPSTLKAGELRTHVSRCQVCMRRT | |
| 394 | MRLLAKIICLMLWAICVAEDCNELPPRRNTEILTGSWSDQTYPEGTQ | CFH |
| | AIYKCRPGYRSLGNVIMVCRKGEWVALNPLRKCQKRPCGHPGDTP | |
| | FGTFTLTGGNVFEYGVKAVYTCNEGYQLLGEINYRECDTDGWTNDI | |
| | PICEVVKCLPVTAPENGKIVSSAMEPDREYHFGQAVRFVCNSGYKIE | |
| | GDEEMHCSDDGFWSKEKPKCVEISCKSPDVINGSPISQKIIYKENERF | |
| | QYKCNMGYEYSERGDAVCTESGWRPLPSCEEKSCDNPYIPNGDYSP | |
| | LRIKHRTGDEITYQCRNGFYPATRGNTAKCTSTGWIPAPRCTLKPCD | |
| | YPDIKHGGLYHENMRRPYFPVAVGKYYSYYCDEHFETPSGSYWDH | |
| | IHCTQDGWSPAVPCLRKCYFPYLENGYNQNYGRKFVQGKSIDVAC | |
| | HPGYALPKAQTTVTCMENGWSPTPRCIRVKTCSKSSIDIENGFISESQ | |
| | YTYALKEKAKYQCKLGYVTADGETSGSITCGKDGWSAQPTCIKSC | |
| | DIPVFMNARTKNDFTWFKLNDTLDYECHDGYESNTGSTTGSIVCGY | |
| | NGWSDLPICYERECELPKIDVHLVPDRKKDQYKVGEVLKFSCKPGF | |
| | TIVGPNSVQCYHFGLSPDLPICKEQVQSCGPPPELLNGNVKEKTKEE | |
| | YGHSEVVEYYCNPRFLMKGPNKIQCVDGEWTTLPVCIVEESTCGDI | |
| | PELEHGWAQLSSPPYYYGDSVEFNCSESFTMIGHRSITCIHGVWTQL | |
| | PQCVAIDKLKKCKSSNLIILEEHLKNKKEFDHNSNIRYRCRGKEGWI | |
| | HTVCINGRWDPEVNCSMAQIQLCPPPPQIPNSHNMTTTLNYRDGEK | |
| | VSVLCQENYLIQEGEEITCKDGRWQSIPLCVEKIPCSQPPQIEHGTINS | |
| | SRSSQESYAHGTKLSYTCEGGFRISEENETTCYMGKWSSPPQCEGLP | |
| | CKSPPEISHGVVAHMSDSYQYGEEVTYKCFEGFGIDGPAIAKCLGEK | |
| | WSHPPSCIKTDCLSLPSFENAIPMGEKKDVYKAGEQVTYTCATYYK | |
| | MDGASNVTCINSRWTGRPTCRDTSCVNPPTVQNAYIVSRQMSKYPS | |
| | GERVRYQCRSP | |
| | YEMFGDEEVMCLNGNWTEPPQCKDSTGKCGPPPPIDNGDITSFPLSV | |
| | YAPASSVEYQCQNLYQLEGNKRITCRNGQWSEPPKCLHPCVISREIM | |
| | ENYNIALRWTAKQKLYSRTGESVEFVCKRGYRLSSRSHTLRTTCWD | |
| | GKLEYPTCAKR | |
| 395 | MEPRPTAPSSGAPGLAGVGETPSAAALAAARVELPGTAVPSVPEDA | SLC12A2 |
| | APASRDGGGVRDEGPAAAGDGLGRPLGPTPSQSRFQVDLVSENAG | |
| | RAAAAAAAAAAAAAAAGAGAGAKQTPADGEASGESEPAKGSEEA | |
| | KGRFRVNFVDPAASSSAEDSLSDAAGVGVDGPNVSFQNGGDTVLSE | |
| | GSSLHSGGGGGSGHHQHYYYDTHTNTYYLRTFGHNTMDAVPRIDH | |
| | YRHTAAQLGEKLLRPSLAELHDELEKEPFEDGFANGEESTPTRDAV | |
| | VTYTAESKGVVKFGWIKGVLVRCMLNIWGVMLFIRLSWIVGQAGI | |
| | GLSVLVIMMATVVTTITGLSTSAIATNGFVRGGGAYYLISRSLGPEF | |
| | GGAIGLIFAFANAVAVAMYVVGFAETVVELLKEHSILMIDEINDIRII | |
| | GAITVVILLGISVAGMEWEAKAQIVLLVILLLAIGDFVIGTFIPLESKK | |
| | PKGFFGYKSEIFNENFGPDFREEETFFSVFAIFFPAATGILAGANISGD | |
| | LADPQSAIPKGTLLAILITTLVYVGIAVSV | |
| | GSCVVRDATGNVNDTIVTELTNCTSAACKLNFDFSSCESSPCSYGL | |
| | MNNFQVMSMVSGFTPLISAGIFSATLSSALASLVSAPKIFQALCKDNI | |
| | YPAFQMFAKGYGKNNEPLRGYILTFLIALGFILIAELNVIAPIISNFFL | |
| | ASYALINFSVFHASLAKSPGWRPAFKYYNMWISLLGAILCCIVMFVI | |
| | NWWAALLTYVIVLGLYIYVTYKKPDVNWGSSTQALTYLNALQHSI | |
| | RLSGVEDHVKNFRPQCLVMTGAPNSRPALLHLVHDFTKNVGLMIC | |
| | GHVHMGPRRQAMKEMSIDQAKYQRWLIKNKMKAFYAPVHADDL | |
| | REGAQYLMQAAGLGRMKPNTLVLGFKKDWLQADMRDVDMYINL | |
| | FHDAFDIQYGVVVIRLKEGLDISHLQGQEELLSSQEKSPGTKDVVVS | |
| | VEYSKKSDLDTSKPLSEKPITHKVEEEDGKTATQPLLKKESKGPIVPL | |
| | NVADQKLLEASTQFQKKQGKNTIDVWWLFDDGGLTLLIPYLLTTK | |
| | KKWKDCKIRVFIGGKINRIDHDRRAMATLLSKFRIDFSDIMVLGDIN | |
| | TKPKKENIIAFEEIIEPYRLHEDDKEQDIADKMKEDEPWRITDNELEL | |
| | YKTKTYRQIRLNELLKEHSSTANIIVMSLPVARKGAVSSALYMAWL | |
| | EALSKDLPPILLVRGNHQSVLTFYS | |
| 396 | MAASKKAVLGPLVGAVDQGTSSTRFLVFNSKTAELLSHHQVEIKQE | GK |
| | FPREGWVEQDPKEILHSVYECIEKTCEKLGQLNIDISNIKAIGVSNQR | |
| | ETTVVWDKITGEPLYNAVVWLDLRTQSTVESLSKRIPGNNNFVKSK | |
| | TGLPLSTYFSAVKLRWLLDNVRKVQKAVEEKRALFGTIDSWLIWSL | |
| | TGGVNGGVHCTDVTNASRTMLFNIHSLEWDKQLCEFFGIPMEILPN | |
| | VRSSSEIYGLMKISHSVKAGALEGVPISGCLGDQSAALVGQMCFQIG | |
| | QAKNTYGTGCFLLCNTGHKCVFSDHGLLTTVAYKLGRDKPVYYAL | |
| | EGSVAIAGAVIRWLRDNLGIIKTSEEIEKLAKEVGTSYGCYFVPAFSG | |
| | LYAPYWEPSARGIICGLTQFTNKCHIAFAALEAVCFQTREILDAMNR | |
| | DCGIPLSHLQVDGGMTSNKILMQLQADILYIPVVKPSMPETTALGAA | |
| | MAAGAAEGVGVWSLEPEDLSAVTMERFEPQINAEESEIRYSTWKK | |
| | AVMKSMGWVTTQSPESGDPSIFCSLPLGF | |
| | FIVSSMVMLIGARYISGIP | |
| 397 | MDVGSKEVLMESPPDYSAAPRGRFGIPCCPVHLKRLLIVVVVVVLIV | SFTPC |
| | VVIVGALLMGLHMSQKHTEMVLEMSIGAPEAQQRLALSEHLVTTA | |
| | TFSIGSTGLVVYDYQQLLIAYKPAPGTCCYIMKIAPESIPSLEALNRK | |
| | VHNFQMECSLQAKPAVPTSKLGQAEGRDAGSAPSGGDPAFLGMAV | |
| | NTLCGEVPLYYI | |
| 398 | MEPGRRGAAALLALLCVACALRAGRAQYERYSFRSFPRDELMPLES | CRTAP |
| | AYRHALDKYSGEHWAESVGYLEISLRLHRLLRDSEAFCHRNCSAAP | |
| | QPEPAAGLASYPELRLFGGLLRRAHCLKRCKQGLPAFRQSQPSREV | |
| | LADFQRREPYKFLQFAYFKANNLPKAIAAAHTFLLKHPDDEMMKR | |
| | NMAYYKSLPGAEDYIKDLETKSYESLFIRAVRAYNGENWRTSITDM | |
| | ELALPDFFKAFYECLAACEGSREIKDFKDFYLSIADHYVEVLECKIQ | |
| | CEENLTPVIGGYPVEKFVATMYHY | |
| | LQFAYYKLNDLKNAAPCAVSYLLFDQNDKVMQQNLVYYQYHRDT | |
| | WGLSDEHFQPRPEAVQFFNVTTLQKELYDFAKENIMDDDEGEVVE | |
| | YVDDLLELEETS | |
| 399 | MAVRALKLLTTLLAVVAAASQAEVESEAGWGMVTPDLLFAEGTA | P3H1 |
| | AYARGDWPGVVLSMERALRSRAALRALRLRCRTQCAADFPWELDP | |
| | DWSPSPAQASGAAALRDLSFFGGLLRRAACLRRCLGPPAAHSLSEE | |
| | MELEFRKRSPYNYLQVAYFKINKLEKAVAAAHTFFVGNPEHMEMQ | |
| | QNLDYYQTMSGVKEADFKDLETQPHMQEFRLGVRLYSEEQPQEAV | |
| | PHLEAALQEYFVAYEECRALCEGPYDYDGYNYLEYNADLFQAITD | |
| | HYIQVLNCKQNCVTELASHPSREKPFEDFLPSHYNYLQFAYYNIGN | |
| | YTQAVECAKTYLLFFPNDEVMNQNLAYYAAMLGEEHTRSIGPRES | |
| | AKEYRQRSLLEKELLFFAYDVFGIPFVDPDSWTPEEVIPKRLQEKQK | |
| | SERETAVRISQEIGNLMKEIETLVEEKTKESLDVSRLTREGGPLLYEG | |
| | ISLTMNSKLLNGSQRVVMDGVISDHECQELQRLTNVAATSGDGYR | |
| | GQTSPHTPNEKFYGVTVFKALKLGQEGKVPLQSAHLYYNVTEKVR | |
| | RIMESYFRLDTPLYFSYSHLVCRTAIEEVQAERKDDSHPVHVDNCIL | |
| | NAETLVCVKEPPAYTFRDYSAILYLNGDFDGGNFYFTELDAKTVTA | |
| | EVQPQCGRAVGFSSGTENPHGVKAVTRGQRCAIALWFTLDPRHSER | |
| | DRVQADDLVKMLFSPEEMDLSQEQPLDAQQGPPEPAQESLSGSESK | |
| | PKDEL | |
| 400 | MTLRLLVAALCAGILAEAPRVRAQHRERVTCTRLYAADIVFLLDGS | COL7A1 |
| | SSIGRSNFREVRSFLEGLVLPFSGAASAQGVRFATVQYSDDPRTEFG | |
| | LDALGSGGDVIRAIRELSYKGGNTRTGAAILHVADHVFLPQLARPG | |
| | VPKVCILITDGKSQDLVDTAAQRLKGQGVKLFAVGIKNADPEELKR | |
| | VASQPTSDFFFFVNDFSILRTLLPLVSRRVCTTAGGVPVTRPPDDSTS | |
| | APRDLVLSEPSSQSLRVQWTAASGPVTGYKVQYTPLTGLGQPLPSE | |
| | RQEVNVPAGETSVRLRGLRPLTEYQVTVIALYANSIGEAVSGTARTT | |
| | ALEGPELTIQNTTAHSLLVAWRSVPGATGYRVTWRVLSGGPTQQQE | |
| | LGPGQGSVLLRDLEPGTDYEVTVSTLFGRSVGPATSLMARTDASVE | |
| | QTLRPVILGPTSILLSWNLVPEARGYRLEWRRETGLEPPQKVVLPSD | |
| | VTRYQLDGLQPGTEYRLTLYTLLEGHEVATPATVVPTGPELPVSPVT | |
| | DLQATELPGQRVRVSWSPVPGATQYRII | |
| | VRSTQGVERTLVLPGSQTAFDLDDVQAGLSYTVRVSARVGPREGSA | |
| | SVLTVRREPETPLAVPGLRVVVSDATRVRVAWGPVPGASGFRISWS | |
| | TGSGPESSQTLPPDSTATDITGLQPGTTYQVAVSVLRGREEGPAAVI | |
| | VARTDPLGPVRTVHVTQASSSSVTITWTRVPGATGYRVSWHSAHGP | |
| | EKSQLVSGEATVAELDGLEPDTEYTVHVRAHVAGVDGPPASVVVR | |
| | TAPEPVGRVSRLQILNASSDVLRITWVGVTGATAYRLAWGRSEGGP | |
| | MRHQILPGNTDSAEIRGLEGGVSY | |
| | SVRVTALVGDREGTPVSIVVTTPPEAPPALGTLHVVQRGEHSLRLR | |
| | WEPVPRAQGFLLHWQPEGGQEQSRVLGPELSSYHLDGLEPATQYR | |
| | VRLSVLGPAGEGPSAEVTARTESPRVPSIELRVVDTSIDSVTLAWTP | |
| | VSRASSYILSWRPLRGPGQEVPGSPQTLPGISSSQRVTGLEPGVSYIFS | |
| | LTPVLDGVRGPEASVTQTPVCPRGLADVVFLPHATQDNAHRAEATR | |
| | RVLERLVLALGPLGPQAVQVGLLSYSHRPSPLFPLNGSHDLGIILQRI | |
| | RDMPYMDPSGNNLGTAVVTAHRYMLAPDAPGRRQHVPGVMVLLV | |
| | DEPLRGDIFSPIREAQASGLNVVMLGMAGADPEQLRRLAPGMDSVQ | |
| | TFFAVDDGPSLDQAVSGLATALCQASFTTQPRPEPCPVYCPKGQKG | |
| | EPGEMGLRGQVGPPGDPGLPGRTGAPGPQGPPGSATAKGERGFPGA | |
| | DGRPGSPGRAGNPGTPGAPGLKGSPGLPGPRGDPGERGPRGPKGEP | |
| | GAPGQVIGGEGPGLPGRKGDPGPSGPPGPRGPLGDPGPRGPPGLPGT | |
| | AMKGDKGDRGERGPPGPGEGGIAPGEPGLPGLPGSPGPQGPVGPPG | |
| | KKGEKGDSEDGAPGLPGQPGSPGEQGPRGPPGAIGPKGDRGFPGPL | |
| | GEAGEKGERGPPGPAGSRGLPGVAGRPGAKGPEGPPGPTGRQGEKG | |
| | EPGRPGDPAVVGPAVAGPKGEKGDVGPAGPRGATGVQGERGPPGL | |
| | VLPGDPGPKGDPGDRGPIGLTGRAGPPGDSGPPGEKGDPGRPGPPGP | |
| | VGPRGRDGEVGEKGDEGPPGDPGLPGKAGERGLRGAPGVRGPVGE | |
| | KGDQGDPGEDGRNGSPGSSGPKGDRGEPGPPGPPGRLVDTGPGARE | |
| | KGEPGDRGQEGPRGPKGDPGLPGAPGERGIEGFRGPPGPQGDPGVR | |
| | GPAGEKGDRGPPGLDGRSGLDGKPGAAGPSGPNGAAGKAGDPGRD | |
| | GLPGLRGEQGLPGPSGPPGLPGKPGEDGKPGLNGKNGEPGDPGEDG | |
| | RKGEKGDSGASGREGRDGPKGERGAPGILGPQGPPGLPGPVGPPGQ | |
| | GFPGVPGGTGPKGDRGETGSKGEQGLPGERGLRGEPGSVPNVDRLL | |
| | ETAGIKASALREIVETWDESSGSFLPVPERRRGPKGDSGEQGPPGKE | |
| | GPIGFPGERGLKGDRGDPGPQGPPGLALGERGPPGPSGLAGEPGKPG | |
| | IPGLPGRAGGVGEAGRPGERGERGEKGERGEQGRDGPPGLPGTPGP | |
| | PGPPGPKVSVDEPGPGLSGEQGPPGLKGAKGEPGSNGDQGPKGDRG | |
| | VPGIKGDRGEPGPRGQDGNPGLPGERGMAGPEGKPGLQGPRGPPGP | |
| | VGGHGDPGPPGAPGLAGPAGPQGPSGLKGEPGETGPPGRGLTGPTG | |
| | AVGLPGPPGPSGLVGPQGSPGLPGQVGETGKPGAPGRDGASGKDG | |
| | DRGSPGVPGSP | |
| | GLPGPVGPKGEPGPTGAPGQAVVGLPGAKGEKGAPGGLAGDLVGE | |
| | PGAKGDRGLPGPRGEKGEAGRAGEPGDPGEDGQKGAPGPKGFKGD | |
| | PGVGVPGSPGPPGPPGVKGDLGLPGLPGAPGVVGFPGQTGPRGEMG | |
| | QPGPSGERGLAGPPGREGIPGPLGPPGPPGSVGPPGASGLKGDKGDP | |
| | GVGLPGPRGERGEPGIRGEDGRPGQEGPRGLTGPPGSRGERGEKGD | |
| | VGSAGLKGDKGDSAVILGPPGPRGAKGDMGERGPRGLDGDKGPRG | |
| | DNGDPGDKGSKGEPGDKGSAGLPGLRGLLGPQGQPGAAGIPGDPGS | |
| | PGKDGVPGIRGEKGDVGFMGPRGLKGERGVKGACGLDGEKGDKG | |
| | EAGPPGRPGLAGHKGEMGEPGVPGQSGAPGKEGLIGPKGDRGFDG | |
| | QPGPKGDQGEKGERGTPGIGGFPGPSGNDGSAGPPGPPGSVGPRGPE | |
| | GLQGQKGERGPPGERVVGAPGVPGAPGERGEQGRPGPAGPRGEKG | |
| | EAALTEDDIRGFVRQEMSQHCACQGQFIASGSRPLPSYAADTAGSQ | |
| | LHAVPVLRVSHAEEEERVPPEDDEYSEYSEYSVEEYQDPEAPWDSD | |
| | DPCSLPLDEGSCTAYTLRWYHRAVTGSTEACHPFVYGGCGGNANR | |
| | FGTREACERRCPPRVVQSQGTGTAQD | |
| 401 | MSIQENISSLQLRSWVSKSQRDLAKSILIGAPGGPAGYLRRASVAQL | PKLR |
| | TQELGTAFFQQQQLPAAMADTFLEHLCLLDIDSEPVAARSTSIIATIG | |
| | PASRSVERLKEMIKAGMNIARLNFSHGSHEYHAESIANVREAVESFA | |
| | GSPLSYRPVAIALDTKGPEIRTGILQGGPESEVELVKGSQVLVTVDPA | |
| | FRTRGNANTVWVDYPNIVRVVPVGGRIYIDDGLISLVVQKIGPEGLV | |
| | TQVENGGVLGSRKGVNLPGAQVDLPGLSEQDVRDLRFGVEHGVDI | |
| | VFASFVRKASDVAAVRAALGPEGHGIKIISKIENHEGVKRFDEILEVS | |
| | DGIMVARGDLGIEIPAEKVFLAQKMMIGRCNLAGKPVVCATQMLES | |
| | MITKPRPTRAETSDVANAVLDGADCIMLSGETAKGNFPVEAVKMQ | |
| | HAIAREAEAAVYHRQLFEELRRAAPLSRDPTEVTAIGAVEAAFKCC | |
| | AAAIIVLTTTGRSAQLLSRYRPRAAVIAVTRSAQAARQVHLCRGVFP | |
| | LLYREPPEAIWADDVDRRVQFGIESG | |
| | KLRGFLRVGDLVIVVTGWRPGSGYTNIMRVLSIS | |
| 402 | MSSPVKRQRMESALDQLKQFTTVVADTGDFHAIDEYKPQDATTNP | TALDO1 |
| | SLILAAAQMPAYQELVEEAIAYGRKLGGSQEDQIKNAIDKLFVLFGA | |
| | EILKKIPGRVSTEVDARLSFDKDAMVARARRLIELYKEAGISKDRILI | |
| | KLSSTWEGIQAGKELEEQHGIHCNMTLLFSFAQAVACAEAGVTLISP | |
| | FVGRILDWHVANTDKKSYEPLEDPGVKSVTKIYNYYKKFSYKTIVM | |
| | GASFRNTGEIKALAGCDFLTISPKLLGELLQDNAKLVPVLSAKAAQA | |
| | SDLEKIHLDEKSFRWLHNEDQMAVEKLSDGIRKFAADAVKLERML | |
| | TERMFNAENGK | |
| 403 | MRLAVGALLVCAVLGLCLAVPDKTVRWCAVSEHEATKCQSFRDH | TF |
| | MKSVIPSDGPSVACVKKASYLDCIRAIAANEADAVTLDAGLVYDAY | |
| | LAPNNLKPVVAEFYGSKEDPQTFYYAVAVVKKDSGFQMNQLRGK | |
| | KSCHTGLGRSAGWNIPIGLLYCDLPEPRKPLEKAVANFFSGSCAPCA | |
| | DGTDFPQLCQLCPGCGCSTLNQYFGYSGAFKCLKDGAGDVAFVKH | |
| | STIFENLANKADRDQYELLCLDNTRKPVDEYKDCHLAQVPSHTVVA | |
| | RSMGGKEDLIWELLNQAQEHFGKDKSKEFQLFSSPHGKDLLFKDSA | |
| | HGFLKVPPRMDAKMYLGYEYVTAIRNLREGTCPEAPTDECKP | |
| | VKWCALSHHERLKCDEWSVNSVGKIECVSAETTEDCIAKIMNGEA | |
| | DAMSLDGGFVYIAGKCGLVPVLAENYNKSDNCEDTPEAGYFAIAV | |
| | VKKSASDLTWDNLKGKKSCHTAVGRTAGWNIPMGLLYNKINHCRF | |
| | DEFFSEGCAPGSKKDSSLCKLCMGSGLNLCEPNNKEGYYGYTGAFR | |
| | CLVEKGDVAFVKHQTVPQNTGGKNPDPWAKNLNEKDYELLCLDG | |
| | TRKPVEEYANCHLARAPNHAVVTRKDKEACVHKILRQQQHLFGSN | |
| | VTDCSGNFCLFRSETKDLLFRDDTVCLAKLHDRNTYEKYLGEEYVK | |
| | AVGNLRKCSTSSLLEACTFRRP | |
| 404 | MAPPQVLAFGLLLAAATATFAAAQEECVCENYKLAVNCFVNNNRQ | EPCAM |
| | CQCTSVGAQNTVICSKLAAKCLVMKAEMNGSKLGRRAKPEGALQN | |
| | NDGLYDPDCDESGLFKAKQCNGTSMCWCVNTAGVRRTDKDTEITC | |
| | SERVRTYWIIIELKHKAREKPYDSKSLRTALQKEITTRYQLDPKFITSI | |
| | LYENNVITIDLVQNSSQKTQNDVDIADVAYYFEKDVKGESLFHSKK | |
| | MDLTVNGEQLDLDPGQTLIYYVDEKAPEFSMQGLKAGVIAVIVVVV | |
| | IAVVAGIVVLVISRKKRMAKYEKA | |
| | EIKEMGEMHRELNA | |
| 405 | MPRRAENWDEAEVGAEEAGVEEYGPEEDGGEESGAEESGPEESGPE | VHL |
| | ELGAEEEMEAGRPRPVLRSVNSREPSQVIFCNRSPRVVLPVWLNFD | |
| | GEPQPYPTLPPGTGRRIHSYRGHLWLFRDAGTHDGLLVNQTELFVPS | |
| | LNVDGQPIFANITLPVYTLKERCLQVVRSLVKPENYRRLDIVRSLYE | |
| | DLEDHPNVQKDLERLTQERIAHQRMGD | |
| 406 | MKRVLVLLLAVAFGHALERGRDYEKNKVCKEFSHLGKEDFTSLSL | GC |
| | VLYSRKFPSGTFEQVSQLVKEVVSLTEACCAEGADPDCYDTRTSAL | |
| | SAKSCESNSPFPVHPGTAECCTKEGLERKLCMAALKHQPQEFPTYV | |
| | EPTNDEICEAFRKDPKEYANQFMWEYSTNYGQAPLSLLVSYTKSYL | |
| | SMVGSCCTSASPTVCFLKERLQLKHLSLLTTLSNRVCSQYAAYGEK | |
| | KSRLSNLIKLAQKVPTADLEDVLPLAEDITNILSKCCESASEDCMAK | |
| | ELPEHTVKLCDNLSTKNSKFEDCCQEKTAMDVFVCTYFMPAAQLPE | |
| | LPDVELPTNKDVCDPGNTKVMDKYTFELSRRTHLPEVFLSKVLEPT | |
| | LKSLGECCDVEDSTTCFNAKGPLLKKELSSFIDKGQELCADYSENTF | |
| | TEYKKKLAERLKAKLPDATPTELAKLVNKHSDFASNCCSINSPPLYC | |
| | DSEIDAELKNIL | |
| 407 | MPSSVSWGILLLAGLCCLVPVSLAEDPQGDAAQKTDTSHHDQDHPT | SERPINA1 |
| | FNKITPNLAEFAFSLYRQLAHQSNSTNIFFSPVSIATAFAMLSLGTKA | |
| | DTHDEILEGLNFNLTEIPEAQIHEGFQELLRTLNQPDSQLQLTTGNGL | |
| | FLSEGLKLVDKFLEDVKKLYHSEAFTVNFGDTEEAKKQINDYVEKG | |
| | TQGKIVDLVKELDRDTVFALVNYIFFKGKWERPFEVKDTEEEDFHV | |
| | DQVTTVKVPMMKRLGMFNIQHCKKLSSWVLLMKYLGNATAIFFLP | |
| | DEGKLQHLENELTHDIITKFLENEDRRSASLHLPKLSITGTYDLKSVL | |
| | GQLGITKVFSNGADLSGVTEEAPLKLSKAVHKAVLTIDEKGTEAAG | |
| | AMFLEAIPMSIPPEVKFNKPFVFLMIEQNTKSPLFMGKVVNPTQK | |
| 408 | MAAPAEPCAGQGVWNQTEPEPAATSLLSLCFLRTAGVWVPPMYL | ABCC6 |
| | WVLGPIYLLFIHHHGRGYLRMSPLFKAKMVLGFALIVLCTSSVAVA | |
| | LWKIQQGTPEAPEFLIHPTVWLTTMSFAVFLIHTERKKGVQSSGVLF | |
| | GYWLLCFVLPATNAAQQASGAGFQSDPVRHLSTYLCLSLVVAQFV | |
| | LSCLADQPPFFPEDPQQSNPCPETGAAFPSKATFWWVSGLVWRGYR | |
| | RPLRPKDLWSLGRENSSEELVSRLEKEWMRNRSAARRHNKAIAFKR | |
| | KGGSGMKAPETEPFLRQEGSQWRPLL | |
| | KAIWQVFHSTFLLGTLSLIISDVFRFTVPKLLSLFLEFIGDPKPPAWKG | |
| | YLLAVLMFLSACLQTLFEQQNMYRLKVLQMRLRSAITGLVYRKVL | |
| | ALSSGSRKASAVGDVVNLVSVDVQRLTESVLYLNGLWLPLVWIVV | |
| | CFVYLWQLLGPSALTAIAVFLSLLPLNFFISKKRNHHQEEQMRQKDS | |
| | RARLTSSILRNSKTIKFHGWEGAFLDRVLGIRGQELGALRTSGLLFS | |
| | VSLVSFQVSTFLVALVVFAVHTLVAENAMNAEKAFVTLTVLNILNK | |
| | AQAFLPFSIHSLVQARVSFDRLVTFLCLEEVDPGVVDSSSSGSAAGK | |
| | DCITIHSATFAWSQESPPCLHRINLTVPQGCLLAVVGPVGAGKSSLLS | |
| | ALLGELSKVEGFVSIEGAVAYVPQEAWVQNTSVVENVCFGQELDPP | |
| | WLERVLEACALQPDVDSFPEGIHTSIGEQGMNLSGGQKQRLSLARA | |
| | VYRKAAVYLLDDPLAALDAHVGQHVFNQVIGPGGLLQGTTRILVT | |
| | HALHILPQADWIIVLANGAIAEMGSYQELLQRKGALMCLLDQARQP | |
| | GDRGEGETEPGTSTKDPRGTSAGRRPELRRERSIKSVPEKDRTTSEA | |
| | QTEVPLDDPDRAGWPAGKDSIQYGRVKATVHLAYLRAVGTPLCLY | |
| | ALFLFLCQQVASFCRGYWLSLWADDPAVGGQQTQAALRGGIFGLL | |
| | GCLQAIGLFASMAAVLLGGARASRLLFQRLLWDVVRSPISFFERTPI | |
| | GHLLNRFSKETDTVDVDIPDKLRSLLMYAFGLLEVSLVVAVATPLA | |
| | TVAILPLFLLYAGFQSLYVVSSCQLRRLESASYSSVCSHMAETFQGS | |
| | TVVRAF | |
| | RTQAPFVAQNNARVDESQRISFPRLVADRWLAANVELLGNGLVFA | |
| | AATCAVLSKAHLSAGLVGFSVSAALQVTQTLQWVVRNWTDLENSI | |
| | VSVERMQDYAWTPKEAPWRLPTCAAQPPWPQGGQIEFRDFGLRYR | |
| | PELPLAVQGVSFKIHAGEKVGIVGRTGAGKSSLASGLLRLQEAAEG | |
| | GIWIDGVPIAHVGLHTLRSRISIIPQDPILFPGSLRMNLDLLQEHSDEA | |
| | IWAALETVQLKALVASLPGQLQYKCADRGEDLSVGQKQLLCLARA | |
| | LLRKTQILILDEATAAVDPGTELQM | |
| | QAMLGSWFAQCTVLLIAHRLRSVMDCARVLVMDKGQVAESGSPA | |
| | QLLAQKGLFYRLAQESGLV | |
| 409 | MQIELSTCFFLCLLRFCFSATRRYYLGAVELSWDYMQSDLGELPVD | F8 |
| | ARFPPRVPKSFPFNTSVVYKKTLFVEFTDHLFNIAKPRPPWMGLLGP | |
| | TIQAEVYDTVVITLKNMASHPVSLHAVGVSYWKASEGAEYDDQTS | |
| | QREKEDDKVFPGGSHTYVWQVLKENGPMASDPLCLTYSYLSHVDL | |
| | VKDLNSGLIGALLVCREGSLAKEKTQTLHKFILLFAVFDEGKSWHSE | |
| | TKNSLMQDRDAASARAWPKMHTVNGYVNRSLPGLIGCHRKSVYW | |
| | HVIGMGTTPEVHSIFLEGHTFLVRNHRQASLEISPITFLTAQTLLMDL | |
| | GQFLLFCHISSHQHDGMEAYVKVDSCPEPQLRMKNNEEAEDYDD | |
| | DLTDSEMDVVRFDDDNSPSFIQIRSVAKKHPKTWVHYIAAEEEDWD | |
| | YAPLVLAPDDRSYKSQYLNNGPQRIGRKYKKVRFMAYTDETFKTR | |
| | EAIQHESGILGPLLYGEVGDTLLIIFKNQASRPYNIYPHGITDVRPLYS | |
| | RRLPKGVKHLKDFPILPGEIFKYKWTVTVEDGPTKSDPRCLTRYYSS | |
| | FVNMERDLASGLIGPLLICYKESVDQRGNQIMSDKRNVILFSVFDEN | |
| | RSWYLTENIQRFLPNPAGVQLEDPEFQASNIMHSINGYVFDSLQLSV | |
| | CLHEVAYWYILSIGAQTDFLSVFFSGYTFKHKMVYEDTLTLFPFSGE | |
| | TVFMSMENPGLWILGCHNSDFRNRGMTALLKVSSCDKNTGDYYED | |
| | SYEDISAYLLSKNNAIEPRSFSQNSRHPSTRQKQFNATTIPENDIEKTD | |
| | PWFAHRTPMPKIQNVSSSDLLMLLRQSPTPHGLSLSDLQEAKYETFS | |
| | DDPS | |
| | PGAIDSNNSLSEMTHFRPQLHHSGDMVFTPESGLQLRLNEKLGTTA | |
| | ATELKKLDFKVSSTSNNLISTIPSDNLAAGTDNTSSLGPPSMPVHYDS | |
| | QLDTTLFGKKSSPLTESGGPLSLSEENNDSKLLESGLMNSQESSWGK | |
| | NVSSTESGRLFKGKRAHGPALLTKDNALFKVSISLLKTNKTSNNSAT | |
| | NRKTHIDGPSLLIENSPSVWQNILESDTEFKKVTPLIHDRMLMDKNA | |
| | TALRLNHMSNKTTSSKNMEMVQQKKEGPIPPDAQNPDMSFFKMLF | |
| | LPESARWIQRTHGKNSLNSGQGPSPKQLVSLGPEKSVEGQNFLSEKN | |
| | KVVVGKGEFTKDVGLKEMVFPSSRNLFLTNLDNLHENNTHNQEKK | |
| | IQEEIEKKETLIQENVVLPQIHTVTGTKNFMKNLFLLSTRQNVEGSYD | |
| | GAYAPVLQDFRSNDSTNRTKKHTAHFSKKGEEENLEGLGNQTKQI | |
| | VEKYACTTRISPNTSQQNFVTQRSKRALKQFRLPLEETELEKRIIVDD | |
| | TSTQWSKNMKHLTPSTLTQIDYNEKE | |
| | KGAITQSPLSDCLTRSHSIPQANRSPLPIAKVSSFPSIRPIYLTRVLFQD | |
| | NSSHLPAASYRKKDSGVQESSHFLQGAKKNNLSLAILTLEMTGDQR | |
| | EVGSLGTSATNSVTYKKVENTVLPKPDLPKTSGKVELLPKVHIYQK | |
| | DLFPTETSNGSPGHLDLVEGSLLQGTEGAIKWNEANRPGKVPFLRV | |
| | ATESSAKTPSKLLDPLAWDNHYGTQIPKEEWKSQEKSPEKTAFKKK | |
| | DTILSLNACESNHAIAAINEGQNKPEIEVTWAKQGRTERLCSQNPPV | |
| | LKRHQREITRTTLQSDQEEIDYDDTISVEMKKEDFDIYDEDENQSPR | |
| | SFQKKTRHYFIAAVERLWDYGMSSSPHVLRNRAQSGSVPQFKKVVF | |
| | QEFTDGSFTQPLYRGELNEHLGLLGPYIRAEVEDNIMVTFRNQASRP | |
| | YSFYSSLISYEEDQRQGAEPRKNFVKPNETKTYFWKVQHHMAPTKD | |
| | EFDCKAWAYFSDVDLEKDVHSGLIGPLLVCHTNTLNPAHGRQVTV | |
| | QEFALFFTIFDETKSWYFTENMERNCRA | |
| | PCNIQMEDPTFKENYRFHAINGYIMDTLPGLVMAQDQRIRWYLLSM | |
| | GSNENIHSIHFSGHVFTVRKKEEYKMALYNLYPGVFETVEMLPSKA | |
| | GIVVRVECLIGEHLHAGMSTLFLVYSNKCQTPLGMASGHIRDFQITAS | |
| | GQYGQWAPKLARLHYSGSINAWSTKEPFSWIKVDLLAPMIIHGIKT | |
| | QGARQKFSSLYISQFIIMYSLDGKKWQTYRGNSTGTLMVFFGNVDS | |
| | SGIKHNIFNPPIIARYIRLHPTHYSIRSTLRMELMGCDLNSCSMPLGM | |
| | ESKAISDAQITASSYFTNMFATWSPSKARLHLQGRSNAWRPQVNNP | |
| | KEWLQVDFQKTMKVTGVTTQGVKSLLTSMYVKEFLISSSQDGHQW | |
| | TLFFQNGKVKVFQGNQDSFTPVVNSLDPPLLTRYLRIHPQSWVHQIA | |
| | LRMEVLGCEAQDLY | |
| 410 | MQRVNMIMAESPGLITICLLGYLLSAECTVFLDHENANKILNRPKRY | F9 |
| | NSGKLEEFVQGNLERECMEEKCSFEEAREVFENTERTTEFWKQYVD | |
| | GDQCESNPCLNGGSCKDDINSYECWCPFGFEGKNCELDVTCNIKNG | |
| | RCEQFCKNSADNKVVCSCTEGYRLAENQKSCEPAVPFPCGRVSVSQ | |
| | TSKLTRAETVFPDVDYVNSTEAETILDNITQSTQSFNDFTRVVGGED | |
| | AKPGQFPWQVVLNGKVDAFCGGSIVNEKWIVTAAHCVETGVKITV | |
| | VAGEHNIEETEHTEQKRNVIRII | |
| | PHHNYNAAINKYNHDIALLELDEPLVLNSYVTPICIADKEYTNIFLKF | |
| | GSGYVSGWGRVFHKGRSALVLQYLRVPLVDRATCLRSTKFTIYNN | |
| | MFCAGFHEGGRDSCQGDSGGPHVTEVEGTSFLTGIISWGEECAMKG | |
| | KYGIYTKVSRYVNwIKEKTKLT | |
| 411 | MDPPRPALLALLALPALLLLLLAGARAEEEMLENVSLVCPKDATRF | ApoB |
| | KHLRKYTYNYEAESSSGVPGTADSRSATRINCKVELEVPQLCSFILK | |
| | TSQCTLKEVYGFNPEGKALLKKTKNSEEFAAAMSRYELKLAIPEGK | |
| | QVFLYPEKDEPTYILNIKRGIISALLVPPETEEAKQVLFLDTVYGNCS | |
| | THFTVKTRKGNVATEISTERDLGQCDRFKPIRTGISPLALIKGMTRPL | |
| | STLIS | |
| | SSQSCQYTLDAKRKHVAEAICKEQHLFLPFSYKNKYGMVAQVTQT | |
| | LKLEDTPKINSRFFGEGTKKMGLAFESTKSTSPPKQAEAVLKTLQEL | |
| | KKLTISEQNIQRANLFNKLVTELRGLSDEAVTSLLPQLIEVSSPITLQA | |
| | LVQCGQPQCSTHILQWLKRVHANPLLIDVVTYLVALIPEPSAQQLRE | |
| | IFNMARDQRSRATLYALSHAVNNYHKTNPTGTQELLDIANYLMEQI | |
| | QDDCTGDEDYTYLILRVIGNMGQTMEQLTPELKSSILKCVQSTKPSL | |
| | MIQKAAIQALRKMEPKDKD | |
| | QEVLLQTFLDDASPGDKRLAAYLMLMRSPSQAINKIVQILPWEQNE | |
| | QVKNFVASHIANILNSEELDIQDLKKLVKEALKESQLPTVMDFRKFS | |
| | RNYQLYKSVSLPSLDPASAKIEGNLIFDPNNYLPKESMLKTTLTAFG | |
| | FASADLIEIGLEGKGFEPTLEALFGKQGFFPDSVNKALYWVNGQVP | |
| | DGVSKVLVDHFGYTKDDKHEQDMVNGIMLSVEKLIKDLKSKEVPE | |
| | ARAYLRILGEELGFASLHDLQLLGKLLLMGARTLQGIPQMIGEVIRK | |
| | GSKNDFFLHYIFMENAFELPTGAGLQLQISSSGVIAPGAKAGVKLEV | |
| | ANMQAELVAKPSVSVEFVTNMGIIIPDFARSGVQMNTNFFHESGLE | |
| | AHVALKAGKLKFIIPSPKRPVKLLSGGNTLHLVSTTKTEVIPPLIENR | |
| | QSWSVCKQVFPGLNYCTSGAYSNASSTDSASYYPLTGDTRLELELR | |
| | PTGEIEQYSVSATYELQREDRALVDTLKFVTQAEGAKQTEATMTFK | |
| | YNRQSMTLSSEVQIPDFDVDLGTILRVN | |
| | DESTEGKTSYRLTLDIQNKKITEVALMGHLSCDTKEERKIKGVISIPR | |
| | LQAEARSEILAHWSPAKLLLQMDSSATAYGSTVSKRVAWHYDEEKI | |
| | EFEWNTGTNVDTKKMTSNFPVDLSDYPKSLHMYANRLLDHRVPQT | |
| | DMTFRHVGSKLIVAMSSWLQKASGSLPYTQTLQDHLNSLKEFNLQ | |
| | NMGLPDFHIPENLFLKSDGRVKYTLNKNSLKIEIPLPFGGKSSRDLK | |
| | MLETVRTPALHFKSVGFHLPSREFQVPTFTIPKLYQLQVPLLGVLDL | |
| | STNVYSNLYNWSASYSGGNTST | |
| | DHFSLRARYHMKADSVVDLLSYNVQGSGETTYDHKNTFTLSYDGS | |
| | LRHKFLDSNIKFSHVEKLGNNPVSKGLLIFDASSSWGPQMSASVHLD | |
| | SKKKQHLFVKEVKIDGQFRVSSFYAKGTYGLSCQRDPNTGRLNGES | |
| | NLRFNSSYLQGTNQITGRYEDGTLSLTSTSDLQSGIIKNTASLKYENY | |
| | ELTLKSDTNGKYKNFATSNKMDMTFSKQNALLRSEYQADYESLRF | |
| | FSLLSGSLNSHGLELNADILGTDKINSGAHKATLRIGQDGISTSATTN | |
| | LKCSLLVLENELNAELGLSGASMKLTTNGRFREHNAKFSLDGKAAL | |
| | TELSLGSAYQAMILGVDSKNIFNFKVSQEGLKLSNDMMGSYAEMK | |
| | FDHTNSLNIAGLSLDFSSKLDNIYSSDKFYKQTVNLQLQPYSLVTTL | |
| | NSDLKYNALDLTNNGKLRLEPLKLHVAGNLKGAYQNNEIKHIYAIS | |
| | SAALSASYKADTVAKVQGVEFSHRLNTDIAGLASAIDMSTNYNSDS | |
| | LHFSNVFRSVMAPFTMTIDAHTNGNGKLALWGEHTGQLYSKFLLK | |
| | AEPLAFTFSHDYKGSTSHHLVSRKSISAALEHKVSALLTPAEQTGTW | |
| | KLKTQFNNNEYSQDLDAYNTKDKIGVELTGRTLADLTLLDSPIKVPL | |
| | LLSEPINIIDALEMRDAVEKPQEFTIVAFVKYDKNQDVHSINLPFFET | |
| | LQEYFERNRQTIIVVLENVQRNLKHINIDQFVRKYRAALGKLPQQA | |
| | NDYLNSFNWERQVSHAKEKLTALTKKYRITENDIQIALDDAKINFNE | |
| | KLSQLQTYMIQFDQYIKDSYDLHDLKIAIANIIDEIIEKLKSLDEHYHI | |
| | RVNLVKTIHDLHLFIENIDFNKSGSSTASWIQNVDTKYQIRIQIQEKL | |
| | QQLKRHIQNIDIQHLAGKLKQHIEAIDVRVLLDQLGTTISFERINDILE | |
| | HVKHFVINLIGDFEVAEKINAFRAKVHELIERYEVDQQIQVLMDKLV | |
| | ELAHQYKLKETIQKLSNVLQQVKIKDYFEKLVGFIDDAVKKLNELSF | |
| | KTFIEDVNKFLDMLIKKLKSFDYHQFVDETNDKIREVTQRLNGEIQA | |
| | LELPQKAEALKLFLEETKATVAVYLESLQDTKITLIINWLQEALSSAS | |
| | LAHMKAKFRETLEDTRDRMYQMDIQQELQRYLSLVGQVYSTLVTY | |
| | ISDWWTLAAKNLTDFAEQYSIQDWAKRMKALVEQGFTVPEIKTILG | |
| | TMPAFEVSLQALQKATFQTPDFIVPLTDLRIPSVQINFKDLKNIKIPSR | |
| | FSTPEFTILNTFHIPSFTIDFVEMKVKIIRTIDQMLNSELQWPVPDIYLR | |
| | DLKVEDIPLARITLPDFRLPEIAIPEFIIPTLNLNDFQVPDLHIPEFQLPH | |
| | ISHTIEVPTFGKLYSILKIQSPLFTLDANADIGNGTTSANEAGIAASITA | |
| | KGESKLEVLNFDFQANAQLSNPKINPLALKESVKFSSKYLRTEHGSE | |
| | MLFFGNAIEGKSNTVASLHTEKNTLELSNGVIVKINNQLTLDSNTKY | |
| | FHKLNIPKLDFSSQADLRNEIKTLLKAGHIAWTSSGKGSWKWACPR | |
| | FSDEGTHESQISFTIEGPLTSFGLSNKINSKHLRVNQNLVYESGSLNFS | |
| | KLEIQSQVDSQHVGHSVLTAKGMALFGEGKAEFTGRHDAHLNGKV | |
| | IGTLKNSLFFSAQPFEITASTNNEGNLKVRFPLRLTGKIDFLNNYALF | |
| | LSPSAQQASWQVSARFNQYKYNQNFSAGNNENIMEAHVGINGE | |
| | ANLDFLNIPLTIPEMRLPYTIITTPPLKDFSLWEKTGLKEFLKTTKQSF | |
| | DLSVKAQYKKNKHRHSITNPLAVLCEFISQSIKSFDRHFEKNRNNAL | |
| | DFVTKSYNETKIKFDKYKAEKSHDELPRTFQIPGYTVPVVNVEVSPF | |
| | TIEMSAFGYVFPKAVSMPSFSILGSDVRVPSYTLILPSLELPVLHVPR | |
| | NLKLSLPDFKELCTISHIFIPAMGNITYDFSFKSSVITLNTNAELFNQS | |
| | DIVAHLLSSSSSVIDALQYKLEGTTRLTRKRGLKLATALSLSNKFVE | |
| | GSHNSTVSLTTKNMEVSVATTTKAQIPILRMNFKQELNGNTKSKPT | |
| | VSSSMEFKYDFNSSMLYSTAKGAVDHKLSLESLTSYFSIESSTKGDV | |
| | KGSVLSREYSGTIASEANTYLNSKSTRSSVKLQGTSKIDDIWNLEVK | |
| | ENFAGEATLQRIYSLWEHSTKNHLQLEGLFFTNGEHTSKATLELSPW | |
| | QMSALV | |
| | QVHASQPSSFHDFPDLGQEVALNANTKNQKIRWKNEVRIHSGSFQS | |
| | QVELSNDQEKAHLDIAGSLEGHLRFLKNIILPVYDKSLWDFLKLDVT | |
| | TSIGRRQHLRVSTAFVYTKNPNGYSFSIPVKVLADKFIIPGLKLNDLN | |
| | SVLVMPTFHVPFTDLQVPSCKLDFREIQIYKKLRTSSFALNLPTLPEV | |
| | KFPEVDVLTKYSQPEDSLIPFFEITVPESQLTVSQFTLPKSVSDGIAAL | |
| | DL | |
| | NAVANKIADFELPTIIVPEQTIEIPSIKFSVPAGIVIPSFQALTARFEVDS | |
| | PVYNATWSASLKNKADYVETVLDSTCSSTVQFLEYELNVLGTHKIE | |
| | DGTLASKTKGTFAHRDFSAEYEEDGKYEGLQEWEGKAHLNIKSPAF | |
| | TDLHLRYQKDKKGISTSAASPAVGTVGMDMDEDDDFSKWNFYYSP | |
| | QSSPDKKLTIFKTELRVRESDEETQIKVNWEEEAASGLLTSLKDNVP | |
| | KATGVLYDYVNKYHWEHTGLTLREVSSKLRRNLQNNAEWVYQGA | |
| | IRQIDDIDVRFQKAASGTTGT | |
| | YQEWKDKAQNLYQELLTQEGQASFQGLKDNVFDGLVRVTQEFHM | |
| | KVKHLIDSLIDFLNFPRFQFPGKPGIYTREELCTMFIREVGTVLSQVY | |
| | SKVHNGSEILFSYFQDLVITLPFELRKHKLIDVISMYRELLKDLSKEA | |
| | QEVFKAIQSLKTTEVLRNLQDLLQFIFQLIEDNIKQLKEMKFTYLINY | |
| | IQDEINTIFSDYIPYVFKLLKENLCLNLHKFNEFIQNELQEASQELQQI | |
| | HQY | |
| | IMALREEYFDPSIVGWTVKYYELEEKIVSLIKNLLVALKDFHSEYIVS | |
| | ASNFTSQLSSQVEQFLHRNIQEYLSILTDPDGKGKEKIAELSATAQEII | |
| | KSQAIATKKIISDYHQQFRYKLQDFSDQLSDYYEKFIAESKRLIDLSI | |
| | QNYHTFLIYITELLKKLQSTTVMNPYMKLAPGELTIIL | |
| 412 | MGTVSSRRSWWPLPLLLLLLLLLGPAGARAQEDEDGDYEELVLAL | PCSK9 |
| | RSEEDGLAEAPEHGTTATFHRCAKDPWRLPGTYVVVLKEETHLSQS | |
| | ERTARRLQAQAARRGYLTKILHVFHGLLPGFLVKMSGDLLELALKL | |
| | PHVDYIEEDSSVFAQSIPWNLERITPPRYRADEYQPPDGGSLVEVYL | |
| | LDTSIQSDHREIEGRVMVTDFENVPEEDGTRFHRQASKCDSHGTHL | |
| | AGVVSGRDAGVAKGASMRSLRVLNCQGKGTVSGTLIGLEFIRKSQL | |
| | VQPVGPLVVLLPLAGGYSRVLNAA | |
| | CQRLARAGVVLVTAAGNFRDDACLYSPASAPEVITVGATNAQDQP | |
| | VTLGTLGTNFGRCVDLFAPGEDIIGASSDCSTCFVSQSGTSQAAAHV | |
| | AGIAAMMLSAEPELTLAELRQRLIHFSAKDVINEAWFPEDQRVLTPN | |
| | LVAALPPSTHGAGWQLFCRTVWSAHSGPTRMATAVARCAPDEELL | |
| | SCSSFSRSGKRRGERMEAQGGKLVCRAHNAFGGEGVYAIARCCLLP | |
| | QANCSVHTAPPAEASMGTRVHCHQQGHVLTGCSSHWEVEDLGTH | |
| | KPPVLRPRGQPNQCVGHREASIHASCCHAPGLECKVKEHGIPAPQE | |
| | QVTVACEEGWTLTGCSALPGTSHVLGAYAVDNTCVVRSRDVSTTG | |
| | STSEGAVTAVAICCRSRHLAQASQELQ | |
| 413 | MDALKSAGRALIRSPSLAKQSWGGGGRHRKLPENWTDTRETLLEG | LDLRAP1 |
| | MLFSLKYLGMTLVEQPKGEELSAAAIKRIVATAKASGKKLQKVTLK | |
| | VSPRGIILTDNLTNQLIENVSIYRISYCTADKMHDKVFAYIAQSQHNQ | |
| | SLECHAFLCTKRKMAQAVTLTVAQAFKVAFEFWQVSKEEKEKRDK | |
| | ASQEGGDVLGARQDCTPSLKSLVATGNLLDLEETAKAPLSTVSANT | |
| | TNMDEVPRPQALSGSSVVWELDDGLDEAFSRLAQSRTNPQVLDTG | |
| | LTAQDMHYAQCLSPVDWDKPDSSGTEQDDLFSF | |
| 414 | MGDLSSLTPGGSMGLQVNRGSQSSLEGAPATAPEPHSLGILHASYSV | ABCG5 |
| | SHRVRPWWDITSCRQQWTRQILKDVSLYVESGQIMCILGSSGSGKT | |
| | TLLDAMSGRLGRAGTFLGEVYVNGRALRREQFQDCFSYVLQSDTL | |
| | LSSLTVRETLHYTALLAIRRGNPGSFQKKVEAVMAELSLSHVADRLI | |
| | GNYSLGGISTGERRRVSIAAQLLQDPKVMLFDEPTTGLDCMTANQI | |
| | VVLLVELARRNRIVVLTIHQPRSELFQLFDKIAILSFGELIFCGTPAEM | |
| | LDFFNDCGYPCPEHSNPFDFYMDLTSVDTQSKEREIETSKRVQMIES | |
| | AYKKSAICHKTLKNIERMKHLKTLPMVPFKTKDSPGVFSKLGVLLR | |
| | RVTRNLVRNKLAVITRLLQNLIMGLFLLFFVLRVRSNVLKGAIQDRV | |
| | GLLYQFVGATPYTGMLNAVNLFPVLRAVSDQESQDGLYQKWQMM | |
| | LAYALHVLPFSVVATMIFSSVCYWTLGLHPEVARFGYFSAALLAPH | |
| | LIGEFLTLVLLGIVQNPNIVNSVVALLSIAGVLVGSGFLRNIQEMPIPF | |
| | KIISYFTFQKYCSEILVVNEFYGLNFTCGSSNVSVTTNPMCAFTQGIQ | |
| | FIEKTCPGATSRFTMNFLILYSFIPALVILGIVVFKIRDHLISR | |
| 415 | MAGKAAEERGLPKGATPQDTSGLQDRLFSSESDNSLYFTYSGQPNT | ABCG8 |
| | LEVRDLNYQVDLASQVPWFEQLAQFKMPWTSPSCQNSCELGIQNLS | |
| | FKVRSGQMLAIIGSSGCGRASLLDVITGRGHGGKIKSGQIWINGQPSS | |
| | PQLVRKCVAHVRQHNQLLPNLTVRETLAFIAQMRLPRTFSQAQRDK | |
| | RVEDVIAELRLRQCADTRVGNMYVRGLSGGERRRVSIGVQLLWNP | |
| | GILILDEPTSGLDSFTAHNLVKTLSRLAKGNRLVLISLHQPRSDIFRLF | |
| | DLVLLMTSGTPIYLGAAQHMVQYFTAIGYPCPRYSNPADFYVDLTSI | |
| | DRRSREQELATREKAQSLAALFLEKVRDLDDFLWKAETKDLDEDT | |
| | CVESSVTPLDTNCLPSPTKMPGAVQQFTTLIRRQISNDFRDLPTLLIH | |
| | GAEACLMSMTIGFLYFGHGSIQLSFMDTAALLFMIGALIPFNVILDVI | |
| | SKCYSERAMLYYELEDGLYTTGPYFFAKILGELPEHCAYIIIYGMPT | |
| | YWLANLRPGLQPFLLHFLLVWLVVFCCRIMALAAAALLPTFHMASF | |
| | FSNALYNSFYLAGGFMINLSSLWTVPAWISKVSFLRWCFEGLMKIQ | |
| | FSRRTYKMPLGNLTIAVSGDKILSVMELDSYPLYAIYLIVIGLSGGFM | |
| | VLYYVSLRFIKQKPSQDW | |
| 416 | MGPPGSPWQWVTLLLGLLLPPAAPFWLLNVLFPPHTTPKAELSNHT | LCAT |
| | RPVILVPGCLGNQLEAKLDKPDVVNWMCYRKTEDFFTIWLDLNMF | |
| | LPLGVDCWIDNTRVVYNRSSGLVSNAPGVQIRVPGFGKTYSVEYLD | |
| | SSKLAGYLHTLVQNLVNNGYVRDETVRAAPYDWRLEPGQQEEYY | |
| | RKLAGLVEEMHAAYGKPVFLIGHSLGCLHLLYFLLRQPQAWKDRFI | |
| | DGFISLGAPWGGSIKPMLVLASGDNQGIPIMSSIKLKEEQRITTTSPW | |
| | MFPSRMAWPEDHVFISTPSFNYTGR | |
| | DFQRFFADLHFEEGWYMWLQSRDLLAGLPAPGVEVYCLYGVGLPT | |
| | PRTYIYDHGFPYTDPVGVLYEDGDDTVATRSTELCGLWQGRQPQPV | |
| | HLLPLHGIQHLNMVFSNLTLEHINAILLGAYRQGPPASPTASPEPPPP | |
| | E | |
| 417 | MKIATVSVLLPLALCLIQDAASKNEDQEMCHEFQAFMKNGKLFCPQ | SPINK5 |
| | DKKFFQSLDGIMFINKCATCKMILEKEAKSQKRARHLARAPKATAP | |
| | TELNCDDFKKGERDGDFICPDYYEAVCGTDGKTYDNRCALCAENA | |
| | KTGSQIGVKSEGECKSSNPEQDVCSAFRPFVRDGRLGCTRENDPVL | |
| | GPDGKTHGNKCAMCAELFLKEAENAKREGETRIRRNAEKDFCKEY | |
| | EKQVRNGRLFCTRESDPVRGPDGRMHGNKCALCAEIFKQRFSEENS | |
| | KTDQNLGKAEEKTKVKREIVKLCSQYQNQAKNGILFCTRENDPIRG | |
| | PDGKMHGNLCSMCQAYFQAENEEKKKAEARARNKRESGKA | |
| | TSYAELCSEYRKLVRNGKLACTRENDPIQGPDGKVHGNTCSMCEVF | |
| | FQAEEEEKKKKEGKSRNKRQSKSTASFEELCSEYRKSRKNGRLFCT | |
| | RENDPIQGPDGKMHGNTCSMCEAFFQQEERARAKAKREAAKEICSE | |
| | FRDQVRNGTLICTREHNPVRGPDGKMHGNKCAMCASVFKLEEEEK | |
| | KNDKEEKGKVEAEKVKREAVQELCSEYRHYVRNGRLPCTRENDPI | |
| | EGLDGKIHGNTCSMCEAFFQQEAKEKERAEPRAKVKREAEKETCDE | |
| | FRRLLQNGKLFCTRENDPVRGPDGKTHGNKCAMCKAVFQKENEER | |
| | KRKEEEDQRNAAGHGSSGGGGGNTQDECAEYREQMKNGRLS | |
| | CTRESDPVRDADGKSYNNQCTMCKAKLEREAERKNEYSRSRSNGT | |
| | GSESGKDTCDEFRSQMKNGKLICTRESDPVRGPDGKTHGNKCTMC | |
| | KEKLEREAAEKKKKEDEDRSNTGERSNTGERSNDKEDLCREFRSM | |
| | QRNGKLICTRENNPVRGPYGKMHINKCAMCQSIFDREANERKKKD | |
| | EEKSSSKPSNNAKDECSEFRNYIRNNELICPRENDPVHGADGKFYTN | |
| | KCYMCRAVFLTEALERAKLQEKPSHVRASQEEDSPDSFSSLDSEMC | |
| | KDYRVLPRIGYLCPKDLKPVCGDDGQTYNNPCMLCHENLIRQTNTH | |
| | IRSTGKCEESSTPGTTAASMPPSDE | |
| 418 | MEKNGNNRKLRVCVATCNRADYSKLAPIMFGIKTEPEFFELDVVVL | GNE |
| | GSHLIDDYGNTYRMIEQDDFDINTRLHTIVRGEDEAAMVESVGLAL | |
| | VKLPDVLNRLKPDIMIVHGDRFDALALATSAALMNIRILHIEGGEVS | |
| | GTIDDSIRHAITKLAHYHVCCTRSAEQHLISMCEDHDRILLAGCPSY | |
| | DKLLSAKNKDYMSIIRMWLGDDVKSKDYIVALQHPVTTDIKHSIKM | |
| | FELTLDALISFNKRTLVLFPNIDAGSKEMVRVMRKKGIEHHPNFRAV | |
| | KHVPFDQFIQLVAHAGCMIGNSSCGVREVGAFGTPVINLGTRQIGRE | |
| | TGENVLHVRDADTQDKILQALHLQFGKQYPCSKIYGDGNAVPRILK | |
| | FLKSIDLQEPLQKKFCFPPVKENISQDIDHILETLSALAVDLGGTNLR | |
| | VAIVSMKGEIVKKYTQFNPKTYEERINLILQMCVEAAAEAVKLNCRI | |
| | LGVGISTGGRVNPREGIVLHSTKLIQEWNSVDLRTPLSDTLHLPVWV | |
| | DNDGNCAALAERKFGQGKGLENFVTL | |
| | ITGTGIGGGIIHQHELIHGSSFCAAELGHLVVSLDGPDCSCGSHGCIE | |
| | AYASGMALQREAKKLHDEDLLLVEGMSVPKDEAVGALHLIQAAKL | |
| | GNAKAQSILRTAGTALGLGVVNILHTMNPSLVILSGVLASHYIHIVK | |
| | DVIRQQALSSVQDVDVVVSDLVDPALLGAASMVLDYTTRRIY | |
| 419 | DIQMTQTTSSLSASLGDRVTISCRASQDISKYLNWYQQKPDGTVKLL | Anti-CD19 scFv |
| | IYHTSRLHSGVPSRFSGSGSGTDYSLTISNLEQEDIATYFCQQGNTLP | (FMC63) |
| | YTFGGGTKLEITGSTSGSGKPGSGEGSTKGEVKLQESGPGLVAPSQS | |
| | LSVTCTVSGVSLPDYGVSWIRQPPRKGLEWLGVIWGSETTYYNSAL | |
| | KSRLTIIKDNSKSQVFLKMNSLQTDDTAIYYCAKHYYYGGSYAMD | |
| | YWGQGTSVTVSS | |
| 420 | DIQMTQTTSSLSASLGDRVTISCRASQDISKYLNWYQQKPDGTVKLL | Anti-CD19 scFv |
| | IYHTSRLHSGVPSRFSGSGSGTDYSLTISNLEQEDIATYFCQQGNTLP | (FMC63) |
| | YTFGGGTKLEITGGGGSGGGGSGGGGSEVKLQESGPGLVAPSQSLS | |
| | VTCTVSGVSLPDYGVSWIRQPPRKGLEWLGVIVVGSETTYYNSALKS | |
| | RLTIIKDNSKSQVFLKMNSLQTDDTAIYYCAKHYYYGGSYAMDYW | |
| | GQGTSVTVSS | |
| 421 | ESKYGPPCPPCP | IgG4 Hinge |
| 422 | TTTPAPRPPTPAPTIASQPLSLRPE | CD8 Hinge |
| 423 | IEVMYPPPYLDNEKSNGTIIHVKGKHLCPSPLFPGPSKP | CD28 |
| 424 | ACRPAAGGAVHTRGLDFACDIYIWAPLAGTCGVLLLSLVITLYC | CD8 |
| 425 | FWVLVVVGGVLACYSLLVTVAFIIFWV | CD28 |
| 426 | FWVLVVVGGVLACYSLLVTVAFIIFWV | CD28 |
| 427 | RSKRSRLLHSDYMNMTPRRPGPTRKHYQPYAPPRDFAAYRS | CD28 |
| 428 | KRGRKKLLYIFKQPFMRPVQTTQEEDGCSCRFPEEEEGGCEL | 4-1BB |
| 429 | RVKFSRSADAPAYQQGQNQLYNELNLGRREEYDVLDKRRGRDPEM | CD3zeta |
| | GGKPRRKNPQEGLYNELQKDKMAEAYSEIGMKGERRRGKGHDGL | |
| | YQGLSTATKDTYDALHMQALPPR | |
| 430 | RVKFSRSADAPAYKQGQNQLYNELNLGRREEYDVLDKRRGRDPEM | CD3zeta |
| | GGKPRRKNPQEGLYNELQKDKMAEAYSEIGMKGERRRGKGHDGL | |
| | YQGLSTATKDTYDALHMQALPPR | |

1. A targeted lipid particle, comprising:
(a) a lipid bilayer enclosing a lumen,
(b) a henipavirus F protein molecule or biologically active portion thereof; and
(c) a targeted envelope protein comprising (i) a henipavirus envelope attachment glycoprotein G (G protein) or a biologically active portion thereof and (ii) single domain antibody (sdAb) variable domain, wherein the sdAb variable domain is attached to the C-terminus of the G protein or the biologically active portion thereof and/or wherein the sdAb is attached to the G protein or the biologically active portion thereof via a peptide linker, wherein the sdAb binds to a cell surface molecule of a target cell,
wherein the F protein molecule or the biologically active portion thereof and the targeted envelope protein are embedded in the lipid bilayer.

2. The targeted lipid particle of claim 1, wherein the cell surface molecule is a protein, glycan, lipid or low molecular weight molecule.

3. The targeted lipid particle of claim 1, wherein the target cell is selected from the group consisting of tumor-infiltrating lymphocytes, T cells, neoplastic or tumor cells, virus-infected cells, stem cells, central nervous system (CNS) cells, hematopoeietic stem cells (HSCs), liver cells and fully differentiated cells.

4. The targeted lipid particle of claim 1, wherein the target cell is selected from the group consisting of a CD3+ T cell, a CD4+ T cell, a CD8+ T cell, a hepatocyte, a haematopoietic stem cell, a CD34+ haematopoietic stem cell, a CD105+ haematopoietic stem cell, a CD117+ haematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, and a CD30+ lung epithelial cell.

5. The targeted lipid particle of claim 1, wherein the single domain antibody binds to an antigen or portion thereof present on a hepatocyte.

6. The targeted lipid particle of claim 1, wherein the cell surface molecule or antigen is selected from the group consisting of ASGR1, ASGR2 and TM4SF.

7. The targeted lipid particle of claim 1, wherein the single domain antibody binds to an antigen or portion thereof present on a T cell.

8. The targeted lipid particle of claim 1, wherein the cell surface molecule or antigen is CD8 or CD4.

9. The targeted lipid particle of claim 1, wherein the cell surface molecule or antigen is low density lipoprotein receptor (LDL-R).

10. A targeted lipid particle, comprising:
(a) a lipid bilayer enclosing a lumen,
(b) a henipavirus F protein molecule or biologically active portion thereof; and
(c) a targeted envelope protein comprising (i) a henipavirus envelope attachment glycoprotein G (G protein) or a biologically active portion thereof and (ii) a binding domain, wherein the binding domain is attached to the C-terminus of the G protein or the biologically active portion thereof, and wherein the binding domain binds a cell surface molecule selected from the group consisting of ASGR1, ASGR2, TM4SF5, CD8, CD4 and low density lipoprotein receptor (LDL-R),
wherein the F protein molecule or the biologically active portion thereof and the targeted envelope protein are embedded in the lipid bilayer.

11-12. (canceled)

13. The targeted lipid particle of claim 1, wherein the lipid particle is a lentiviral vector.

14. A lentiviral vector, comprising:
(a) a henipavirus F protein molecule or biologically active portion thereof; and
(b) a targeted envelope protein comprising (i) a henipavirus envelope attachment glycoprotein G (G protein) or a biologically active portion thereof and (ii) a binding domain, wherein the binding domain is attached to the C-terminus of the G protein or the biologically active portion thereof, and wherein the binding domain binds CD4; and
(c) a cargo comprising nucleic acid encoding a chimeric antigen receptor (CAR), wherein the CAR comprises (i) an extracellular antigen binding domain that binds CD19, (ii) a transmembrane domain and (iii) an intracellular signaling region comprising a CD3zeta signaling domain.

15-16. (canceled)

17. A lentiviral vector, comprising:
(a) a henipavirus F protein molecule or biologically active portion thereof; and
(b) a targeted envelope protein comprising (i) a henipavirus envelope attachment glycoprotein G (G protein) or a biologically active portion thereof and (ii) a binding domain, wherein the binding domain is attached to the C-terminus of the G protein or the biologically active portion thereof, and wherein the binding domain binds a cell surface molecule selected from the group consisting of ASGR1, ASGR2 and TM4SF5.

18-19. (canceled)

20. The lentiviral vector of claim 14, wherein the binding domain is attached to the G protein via a linker.

21. The targeted lipid particle of claim 10, wherein the binding domain is a single domain antibody or is a single chain variable fragment (scFv).

22-23. (canceled)

24. The targeted lipid particle of claim 1, wherein the G protein or the biologically active portion thereof is a wild-type Nipah virus G (NiV-G) protein or a Hendra virus G protein, or is a functionally active variant or biologically active portion thereof.

25-33. (canceled)

34. The targeted lipid particle of claim 1, wherein the mutant NiV-G protein or the biologically active portion has the amino acid sequence set forth in SEQ ID NO: 16 or an amino acid sequence having at or about 80% sequence identity to SEQ ID NO:16.

35. The targeted lipid particle of claim 1, wherein the F protein or the biologically active portion thereof is a wild-type Nipah virus F (NiV-F) protein or a Hendra virus F protein or is a functionally active variant or biologically active portion thereof.

36-39. (canceled)

40. The targeted lipid particle of claim 1, wherein the NiV-F protein is a biologically active portion thereof that has a 22 amino acid truncation at or near the C-terminus of the wild-type NiV-F protein (SEQ ID NO:2).

41. The targeted lipid particle of claim 1, wherein the NiV-F protein or the biologically active portion has the sequence set forth in SEQ ID NO:23 or an amino acid sequence that is encoded by a sequence of nucleotides encoding a sequence having at or about 80% sequence identity to SEQ ID NO:23.

42. The targeted lipid particle of claim 1, wherein the F protein comprises the sequence set forth in SEQ ID NO:23 and the G protein comprises the sequence set forth in SEQ ID NO:16.

43-48. (canceled)

49. The targeted lipid particle of claim 1, wherein the lipid particle further comprises an exogenous agent.

50-54. (canceled)

55. The targeted lipid particle of claim 10, wherein the membrane protein is a chimeric antigen receptor (CAR).

56. (canceled)

57. The targeted lipid particle of claim 10, wherein the exogenous agent is a nucleic acid comprising a payload gene for correcting a genetic deficiency.

58. A polynucleotide comprising a nucleic acid sequence encoding:
(i) a henipavirus envelope attachment glycoprotein G (G protein) or a biologically active portion thereof and (ii) a single domain antibody (sdAb) variable domain, wherein the sdAb variable domain is attached to the C-terminus of the G protein or the biologically active portion thereof; or
(i) a henipavirus envelope attachment glycoprotein G (G protein) or a biologically active portion thereof and (ii) a binding domain that binds a cell surface molecule selected from the group consisting of ASGR1, ASGR2, TM4SF5, CD4, CD8, and low density lipoprotein receptor (LDL-R).

59-90. (canceled)

91. A vector comprising the polynucleotide of claim 58.

92. (canceled)

93. A plasmid comprising the polynucleotide of claim 58.

94. (canceled)

95. A cell comprising the vector of claim 91.

96. A method of making a targeted lipid particle comprising a henipavirus F protein molecule or biologically active portion thereof and a targeted envelope protein comprising a henipavirus envelope attachment glycoprotein G (G protein) or a biologically active portion thereof and a single domain antibody (sdAb) variable domain, the method comprising:
a) providing a cell that comprises a nucleic acid encoding a henipavirus F protein molecule or biologically active portion thereof and a nucleic acid encoding a targeted envelope protein, the targeted envelope protein comprising a henipavirus envelope attachment glycoprotein G (G protein) or a biologically active portion thereof and a single domain antibody (sdAb) variable domain;
b) culturing the cell under conditions that allow for production of a targeted lipid particle, and
c) separating, enriching, or purifying the targeted lipid particle from the cell, thereby making the targeted lipid particle.

97. A method of making a pseudotyped lentiviral vector, the method comprising:
a) providing a producer cell that comprises a lentiviral viral nucleic acid(s), a nucleic acid encoding a henipavirus F protein molecule or biologically active portion thereof, and a nucleic acid encoding a targeted envelope protein, said targeted envelope protein comprising a henipavirus envelope attachment glycoprotein G (G protein) or a biologically active portion thereof and a single domain antibody;
b) culturing the cell under conditions that allow for production of the lentiviral vector, and
c) separating, enriching, or purifying the lentiviral vector from the cell, thereby making the pseudotyped lentiviral vector.

98. A method of making a targeted lipid particle comprising a henipavirus F protein molecule or biologically active portion thereof and a targeted envelope protein comprising a henipavirus envelope attachment glycoprotein G (G protein) or a biologically active portion thereof and a binding domain, the method comprising:
a) providing a cell that comprises a nucleic acid encoding a henipavirus F protein molecule or biologically active portion thereof and a nucleic acid encoding a targeted envelope protein, the targeted envelope protein comprising a henipavirus envelope attachment glycoprotein G (G protein) or a biologically active portion thereof and binding domain, wherein the binding domain:
(i) binds a cell surface molecule selected from the group consisting of ASGR1, ASGR2, and TM4SF5;
(ii) binds a cell surface molecule selected from the group consisting of CD4 or CD8; or
(iii) binds a cell surface molecule that is low density lipoprotein receptor (LDL-R);
b) culturing the cell under conditions that allow for production of a targeted lipid particle, and
c) separating, enriching, or purifying the targeted lipid particle from the cell, thereby making the targeted lipid particle,
wherein the targeted lipid particle is a pseudotyped lentiviral vector.

99-105. (canceled)

106. A producer cell comprising the polynucleotide of claim 58.

107. The producer cell of claim 106, further comprising nucleic acid encoding a henipavirus F protein or a biologically active portion thereof.

108. (canceled)

109. A producer cell comprising (i) a viral nucleic acid(s) and (ii) nucleic acid encoding a henipavirus F protein molecule or biologically active portion thereof and (iii) a nucleic acid encoding a targeted envelope protein comprising a henipavirus envelope attachment glycoprotein G (G protein) or a biologically active portion thereof and a single domain antibody (sdAb) variable domain.

110-113. (canceled)

114. A producer cell comprising (i) a viral nucleic acid(s) and (ii) nucleic acid encoding a henipavirus F protein molecule or biologically active portion thereof and (iii) a nucleic acid encoding a targeted envelope protein comprising a henipavirus envelope attachment glycoprotein G (G protein) or a biologically active portion thereof and a binding domain, wherein the binding domain:
(i) binds a cell surface molecule selected from the group consisting of ASGR1, ASGR2, and TM4SF5;
(ii) binds a cell surface molecule selected from the group consisting of CD4 or CD8; or
(iii) binds a cell surface molecule that is low density lipoprotein receptor (LDL-R).

115-123. (canceled)

124. A targeted lipid particle produced by the method of claim 96.

125-126. (canceled)

127. A composition comprising a plurality of targeted lipid particles of claim 1.

128-129. (canceled)

130. A method of transducing a cell comprising transducing a cell with a lentiviral vector of claim 13.

131. (canceled)

132. A method of delivering an exogenous agent to a subject, the method comprising administering to the subject the targeted lipid particle of claim 49, wherein the targeted lipid particle comprises the exogenous agent.

133. A method of delivering an exogenous agent to a subject, the method comprising administering to the subject the composition of claim 127, wherein targeted lipid particles of the plurality comprise the exogenous agent.

134. A method of delivering a chimeric antigen receptor (CAR) to a cell, comprising contacting a cell with the lentiviral vector of claim 14, wherein the lentiviral vector comprises a nucleic acid encoding the CAR.

135. A method of delivering a chimeric antigen receptor (CAR) to a cell, comprising contacting a cell with the composition of claim 127 wherein targeted lipid particles of the plurality comprise a nucleic acid encoding the CAR.

136. A method of delivering an exogenous agent to a hepatocyte, comprising contacting a cell with the lentiviral vector of claim 17.

137. A method of delivering an exogenous agent to a hepatocyte, comprising contacting a cell with the composition of claim 127, wherein targeted lipid particles of the plurality comprise an exogenous agent for delivery to the hepatocyte.

138. (canceled)

139. A method of treating a disease or disorder in a subject, the method comprising administering to the subject the composition of claim 127.

140. A method of fusing a mammalian cell to a targeted lipid particle, the method comprising administering to the subject the composition of claim 127.

141. (canceled)
